(19) United States
(12) Patent Application Publication
(10) Pub. No.: US 2022/0273659 A1
(43) Pub. Date: Sep. 1, 2022

(54) HETEROCYCLIC COMPOUNDS AS KINASE INHIBITORS

(71) Applicant: NUVATION BIO INC., New York, NY (US)

(72) Inventors: Son Minh PHAM, San Francisco, CA (US); Sarvajit CHAKRAVARTY, Edmond, OK (US); Jayakanth KANKANALA, Plymouth, MN (US); Brahmam PUJALA, Greater Noida (IN); Amit SHETE, Noida (IN); Mukesh GANGAR, Greater Noida (IN); Bhawana BHATT, Uttar Pradesh (IN); Chris P. MILLER, San Mateo, CA (US); Jeremy D. PETTIGREW, Vancouver (CA)

(21) Appl. No.: 16/919,001

(22) Filed: Jul. 1, 2020

Related U.S. Application Data
(60) Provisional application No. 62/870,021, filed on Jul. 2, 2019.

(51) Int. Cl. A61K 31/519; A61P 35/00; C07D 471/04; A61K 39/395

(52) U.S. Cl. CPC A61K 31/519; A61K 39/3955; C07D 471/04; A61P 35/00

(57) ABSTRACT

Heterocyclic compounds as CDK4 or CDK6 or other CDK inhibitors are provided. The compounds may find use as therapeutic agents for the treatment of diseases and may find particular use in oncology.

### CROSS REFERENCE TO RELATED APPLICATIONS

This application claims priority to U.S. Provisional Application No. 62/870,021, filed on Jul. 2, 2019, the content of which is incorporated herein by reference in its entirety.

### FIELD OF THE INVENTION

This disclosure relates generally to therapeutics which play a crucial role in the control of the cell cycle and more particularly, compounds that inhibit cyclin-dependent kinases (CDK). The invention also provides pharmaceutically acceptable compositions comprising compounds of the present invention and methods of using said compositions in the treatment of diseases associated with these pathways.

### BACKGROUND OF THE INVENTION

The cell cycle is a period between the successive divisions of a cell. During this period, the contents of the cell must be accurately replicated. The processes that permit the cell to divide are very precisely controlled by a multitude of enzymatic reactions amongst which the protein kinase-triggered protein phosphorylation plays a major role. In eukaryotes, there are four main stages/phases of cell cycle namely the Gap-1 (G1) phase, Synthesis (S) phase, Gap-2 (G2) and Mitosis (M) phases. An extended phase of Gap-1 phase is coined as Gap-0 (G0) phase or Resting phase (Cancers 2014, 6, 2224-2242).

Uncontrolled proliferation is the hallmark of cancer and other proliferative disorders and abnormal cell cycle regulation is, therefore, common in these diseases. Cyclin-dependent kinases (CDK) constitute a heterodimeric family of serine/threonine protein kinases involved in cell cycle and transcription. They include two main groups: cell cycle CDK and transcriptional CDK. The functionality of CDK depends on specific interactions with regulatory proteins named cyclins which form heterodimeric complexes with their partners. These complexes are important regulators of the cellular processes, especially in the cell cycle progression.

The human proteome contains 20 CDK along with 29 cyclins. CDK1, CDK2, CDK4 and CDK6 are generally considered cell cycle CDK, whereas CDK7, CDK8, CDK9 and CDK11 are mainly involved in transcription regulation (Genome Biol 2014; 15(6):122, Nat Cell Biol 2009; 11(11):1275-6). CDK5 is the prototype of atypical CDK: it is activated by the non-cyclin proteins p35 (or Cdk5R1) and p39 (or Cdk5R2) and has unique post-mitotic functions in neuronal biology, angiogenesis and cell differentiation. Proliferative signals induce the transition from the G0 or G1 phases into S phase through the activation of the structurally related CDK4 and CDK6 [Development, 2013; 140 (15):3079-93, Biochem Pharmacol 2012; 84(8):985-93, Nature 2014; 510(7505):393-6]. The binding of cyclin D to CDK4 and to CDK6 promotes the phosphorylation of the transcriptional repressor retinoblastoma protein (RB1).

CDK hyperactivity is often observed in cancer, reflecting their prominent role in cell cycle and transcription regulation. In cancer cells, the process of cell division becomes unregulated, resulting in uncontrolled growth that leads to the development of a tumor. A number of mechanisms contribute to the dysregulation of the cell cycle in malignant cells, including the amplification and hyperactivity of CDK4/6, or their genomic instability, which might cause CDK4/6 to become oncogenic drivers of cell replication. Usurping these mechanisms, cancer cells can continue to replicate by triggering the G1 to S phase transition. This process appears to be facilitated by a shortening of the G1 phase. In a cancer cell, CDK4/6 antagonizes intrinsic tumor suppression mechanisms including cell senescence and apoptosis, which further augments the growth of a tumor. Cancer cells also upregulate other CDK and cyclins and decrease suppressive mechanisms such as intrinsic CDK inhibitors and tumor suppressor proteins. The overall effect of this type of cell cycle dysregulation is malignant cell proliferation and the development of cancer (Clinical Breast Cancer, 2016, 1526-8209).

Several CDK inhibitors have been reported (such as in WO2011101409 and WO2011101417) or clinically developed. Flavopiridol and R-Roscovitine (Seliciclib), were the first generation of pan-CDK inhibitors with anti-tumor activity attributed to down-regulation of CDK9-mediated anti-apoptotic proteins, especially Mcl-1. Recently, a new generation of CDK inhibitors have been developed, advanced to clinical trials, and approved for certain types of cancer. Dinaciclib, a selective inhibitor of CDK1, CDK2, CDK5, and CDK9, was directed towards refractory chronic lymphocytic leukemia while palbociclib was tested against advanced estrogen receptor (ER)-positive breast cancer as a selective inhibitor of CDK4 and CDK6. The development of more selective second and third generation CDK inhibitors, including specific CDK4/6 inhibitors, has led to a renewed enthusiasm for manipulating the cyclin D1-CDK4/6 axis in cancer treatment. There are three FDA-approved CDK4/6 inhibitors presently: Palbociclib, Ribociclib and Abemaciclib.

The development of therapies, including monotherapies, for treatment of proliferative disorders using a therapeutic targeted generically at CDK, or specifically at dual inhibition of CDK4 and CDK6, is therefore potentially highly desirable.

There is still a need for new CDK4/6 inhibitors. Compounds for the treatment of hyper-proliferative diseases preferably have at least one advantageous property selected from selectivity, potency, stability, pharmacodynamic properties and safety profile. In this regard, a novel class of CDK4/6 inhibitors is provided herein.

### BRIEF SUMMARY OF THE INVENTION

In one embodiment, provided is a compound of Formula (K):

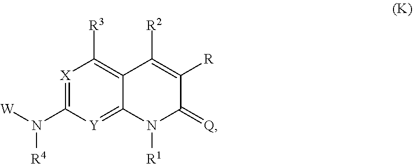

or a salt thereof, wherein X, Y, W, Q, R, R¹, R², R³, and R⁴ are as detailed herein.

In one embodiment, provided is a compound of Formula (J):

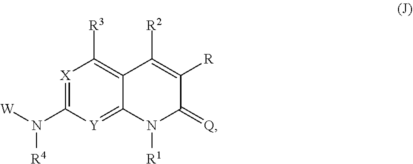

or a salt thereof, wherein X, Y, W, Q, R, R¹, R², R³, and R⁴ are as detailed herein.

In some embodiments, provided is a compound of Formula (I):

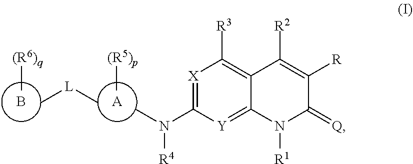

or a salt thereof, wherein X, Y, Q, R, A, B, L, R¹, R², R³, R⁴, R⁵, R⁶, p and q are as detailed herein.

In some embodiments, provided is a compound of Formula (II):

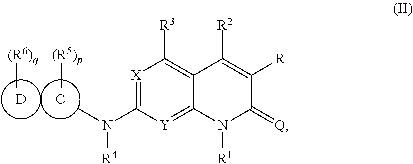

or a salt thereof, wherein X, Y, C, D, R¹, R², R³, R⁴, R⁵, R⁶, p and q are as detailed herein.

In another aspect, provided is a method of treating cancer in an individual in need thereof comprising administering to the individual a therapeutically effective amount of a compound as detailed herein, such as a compound of any one of Formula (J), Formula (I), Formula (II), (I-A), (I-B1) to (I-B22), (I-C1) to (I-C23), or a pharmaceutically acceptable salt thereof. Also provided is a method of modulating CDK4/6 in an individual, comprising administering to the individual a compound detailed herein, or a salt thereof. Also provided is a method of modulating CDK4/6 and one or more of CDK1, CDK2, and CDK9 in an individual, comprising administering to the individual a compound detailed herein, or a salt thereof. Also provided is a method of inhibiting CDK4/6 in a cell, comprising administering a compound detailed herein, or a salt thereof, to the cell. Also provided is a method of inhibiting CDK4/6 and one or more of CDK1, CDK2, and CDK9 in a cell, comprising administering a compound detailed herein, or a salt thereof, to the cell. In some embodiments of the methods detailed herein, the methods comprise administration of a compound detailed herein, or a salt thereof, as a monotherapy.

In another aspect, provided is a pharmaceutical composition comprising a compound detailed herein, or a pharmaceutically acceptable salt thereof, and a pharmaceutically acceptable carrier or excipient. Kits comprising a compound detailed herein, or a salt thereof, are also provided. Kits may optionally include instructions for use, such as instructions for use in any of the methods detailed herein, for example, for use in the treatment of cancer. A compound as detailed herein, or a salt thereof, is also provided for the manufacture of a medicament for the treatment of cancer.

### DETAILED DESCRIPTION OF THE INVENTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted by more than one halogen, it may be referred to by using a prefix corresponding to the number of halogen moieties attached, e.g., dihaloaryl, dihaloalkyl, trihaloaryl etc. refer to aryl and alkyl substituted by two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, thiophenyl, furanyl, thiazolyl, pyrazolyl, oxazolyl, isooxazolyl, imidazolyl, quinolyl, isoquinolyl, benzimidazolyl, benzpyrazolyl, benzotriazolyl, indole, benzothiazyl, benzoxazolyl, benzisoxazolyl, imidazopyridinyl and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, dihydrooxazolyl, dihydroisoxazolyl, dioxolanyl, morpholinyl, dioxanyl, tetrahydrothiophenyl, and the like.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different, provided that the group's normal valence is not exceeded. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

As used herein “CDK” refers to one or more cyclin-dependent kinases. CDK4/6 refers to both CDK4 and CDK6. Thus, inhibitors of CDK4/6 inhibit both CDK4 and CDK6.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that embodiments, aspects and variations described herein also include “consisting” and/or “consisting essentially of” embodiments, aspects and variations.

### Compounds

In one aspect, provided is a compound of Formula (K):

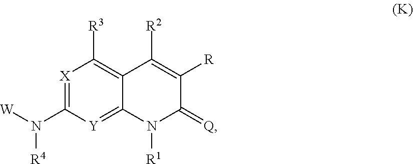

or a salt thereof, wherein:

X is CR^{a} or N, wherein R^{a} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²;

Y is CR^{b} or N, wherein R^{b} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹, or —C(O)NR¹¹R¹²,

provided that at least one of X and Y is N;

Q is O or S;

W is

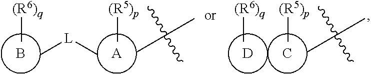

wherein:
A is C₃-C₆ cycloalkyl, 4- to 7-membered heterocyclyl, 5- to 7-membered heteroaryl, or phenyl, each of which is independently optionally substituted by R⁵,
L is a bond, —CR¹¹R¹²—, —O—, —S—, —S(O)₂—, —C(O)—, —NR¹⁰—, —S(O)₂NR¹⁰—, or —NR¹⁰S(O)₂—,
B is hydrogen, C₃-C₆ cycloalkyl, 3- to 10-membered heterocyclyl, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶,
C is C₃-C₆ cycloalkyl, 5- to 7-membered heterocyclyl, 5- to 7-membered heteroaryl, or phenyl, each of which is independently optionally substituted by R⁵, wherein C is fused to D, and
D is C₃-C₆ cycloalkyl, 3- to 7-membered heterocyclyl, 5- to 7-membered heteroaryl, or phenyl, each of which is independently optionally substituted by R⁶;

R is —CN, C₁-C₆ haloalkyl, or C₃-C₆ cycloalkyl;

R¹ is C₁-C₆ alkyl, C₃-C₁₂ cycloalkyl, 3- to 12-membered heterocyclyl, 5- to 10-membered heteroaryl, C₆-C₁₄ aryl, —(C₁-C₃ alkylene)(C₃-C₆ cycloalkyl), —(C₁-C₃ alkylene)(3- to 12-membered heterocyclyl), —C(O)R¹⁰, —(C₁-C₃ alkylene)(5- to 10-membered heteroaryl), or —(C₁-C₃ alkylene)(C₆-C₁₄ aryl), each of which is independently optionally substituted by halogen, oxo, —OR¹³, —NR¹³R¹⁴, —C(O)R¹³, —CN, C₃-C₈ cycloalkyl, or C₁-C₆ alkyl optionally substituted by oxo, —OH or halogen;

R² and R³ are each independently hydrogen, C₁-C₆ alkyl, C₃-C₆ cycloalkyl, C₁-C₆ haloalkyl, C₁-C₆ alkoxy, C₁-C₆ haloalkoxy, halogen, —CN, —C(O)R¹⁰, or —C(O)NR¹¹R¹²;

R⁴ is hydrogen or C₁-C₆ alkyl;

each R⁵ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen;

each R⁶ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen,
or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each optionally substituted by C₁-C₆ alkyl;

R¹⁰ is independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR¹⁵, —NR¹⁵R¹⁶, or C₁-C₆ alkyl optionally substituted by halogen, —OH or oxo;

R¹¹ and R¹² are each independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR¹⁵, —NR¹⁵R¹⁶ or C₁-C₆ alkyl optionally substituted by halogen, —OH or oxo,
or R¹¹ and R¹² are taken together with the atom to which they attached to form a 3- to 6-membered heterocyclyl optionally substituted by halogen, oxo, or C₁-C₆ alkyl optionally substituted by halogen;

R¹³ and R¹⁴ are each independently hydrogen, —OH, C₁-C₆ alkoxy, or C₁-C₆ alkyl, wherein the C₁-C₆ alkyl of R¹³ and R¹⁴ are optionally substituted by halogen, —OR, —NR¹⁵R¹⁶, or oxo,
or R¹³ and R¹⁴ are taken together with the atom to which they attached to form a 3- to 6-membered heterocyclyl optionally substituted by halogen, oxo or C₁-C₆ alkyl optionally substituted by halogen or oxo;

R¹⁵ and R¹⁶ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo,
or R¹⁵ and R¹⁶ are taken together with the atom to which they attached to form a 3- to 6-membered heterocyclyl optionally substituted by halogen, oxo or C₁-C₆ alkyl optionally substituted by oxo or halogen;

p and q are each independently 0, 1, 2, 3 or 4.

In some embodiments, the compound of Formula (K) is a compound of Formula (J):

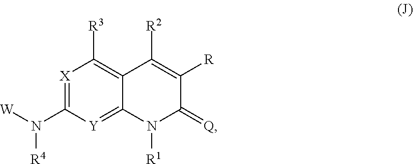

or a salt thereof, wherein:

X is CR^{a} or N, wherein R^{a} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²;

Y is CR^{b} or N, wherein R^{b} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²,

provided that at least one of X and Y is N;

Q is O or S;

W is

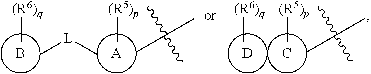

wherein:
A is C₃-C₆ cycloalkyl, 4- to 7-membered heterocyclyl, 5- to 7-membered heteroaryl, or phenyl, each of which is independently optionally substituted by R⁵,
L is a bond, —CR¹¹R¹²—, —O—, —S—, —S(O)₂—, —C(O)—, —NR¹⁰—, —S(O)₂NR¹⁰—, or —NR¹⁰S(O)₂—,
B is hydrogen, C₃-C₆ cycloalkyl, 3- to 10-membered heterocyclyl, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶,
C is C₃-C₆ cycloalkyl, 5- to 7-membered heterocyclyl, 5- to 7-membered heteroaryl, or phenyl, each of which is independently optionally substituted by R⁵, wherein C is fused to D, and
D is C₃-C₆ cycloalkyl, 3- to 7-membered heterocyclyl, 5- to 7-membered heteroaryl, or phenyl, each of which is independently optionally substituted by R⁶;

R is —CN or C₁-C₆ haloalkyl;

R¹ is C₁-C₆ alkyl, C₃-C₆ cycloalkyl, 3- to 12-membered heterocyclyl, 5- to 10-membered heteroaryl, C₆-C₁₄ aryl, —(C₁-C₃ alkylene)(C₃-C₆ cycloalkyl), —(C₁-C₃ alkylene)(3- to 12-membered heterocyclyl), —C(O)R¹⁰, —(C₁-C₃ alkylene)(5- to 10-membered heteroaryl), or —(C₁-C₃ alkylene)(C₆-C₁₄ aryl), each of which is independently optionally substituted by halogen, oxo, —OR¹¹, —NR¹³R¹⁴, —C(O)R¹³, —CN, C₃-C₈ cycloalkyl, or C₁-C₆ alkyl optionally substituted by oxo, —OH or halogen;

R² and R³ are each independently hydrogen, C₁-C₆ alkyl, C₃-C₆ cycloalkyl, C₁-C₆ haloalkyl, C₁-C₆ alkoxy, C₁-C₆ haloalkoxy, halogen, —CN, —C(O)R¹⁰, or —C(O)NR¹¹R¹²;

R⁴ is hydrogen or C₁-C₆ alkyl;

each R⁵ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹²,

C₃-C₆ cycloalkyl, 3- to 12-membered heterocyclyl, —(C₁-C₃ alkylene)OR¹⁰,
—(C₁-C₃ alkylene)SR¹⁰, —(C₁-C₃ alkylene)NR¹¹R¹², —(C₁-C₃ alkylene)C(O)R¹⁰,
—(C₁-C₃ alkylene)C(O)NR¹¹R¹², —(C₁-C₃ alkylene)NR¹⁰C(O)R¹¹,
—(C₁-C₃ alkylene)NR¹⁰C(O)NR¹¹R¹², —(C₁-C₃ alkylene)S(O)₂R¹⁰,
—(C₁-C₃ alkylene)NR¹⁰S(O)₂R¹¹, —(C₁-C₃ alkylene)NR¹⁰S(O)₂NR¹¹R¹²,
—(C₁-C₃ alkylene)S(O)₂NR¹¹R¹², —(C₁-C₃ alkylene)(C₃-C₆ cycloalkyl), or
—(C₁-C₃ alkylene)(3- to 12-membered heterocyclyl), each of which is independently independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen;

each R⁶ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹²,

C₃-C₆ cycloalkyl, 3- to 12-membered heterocyclyl, —(C₁-C₃ alkylene)OR¹⁰,
—(C₁-C₃ alkylene)SR¹⁰, —(C₁-C₃ alkylene)NR¹¹R¹², —(C₁-C₃ alkylene)C(O)R¹⁰,
—(C₁-C₃ alkylene)C(O)NR¹¹R¹², —(C₁-C₃ alkylene)NR¹⁰C(O)R¹¹,
—(C₁-C₃ alkylene)NR¹⁰C(O)NR¹¹R¹², —(C₁-C₃ alkylene)S(O)₂R¹⁰,
—(C₁-C₃ alkylene)NR¹⁰S(O)₂R¹¹, —(C₁-C₃ alkylene)NR¹⁰S(O)₂NR¹¹R¹²,
—(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen,
or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each optionally substituted by C₁-C₆ alkyl;

R¹⁰ is independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR¹⁵, —NR¹⁵R¹⁶, or C₁-C₆ alkyl optionally substituted by halogen, —OH or oxo;

R¹¹ and R¹² are each independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR¹⁵, —NR¹⁵R¹⁶ or C₁-C₆ alkyl optionally substituted by halogen, —OH or oxo,
or R¹¹ and R¹² are taken together with the atom to which they attached to form a 3- to 6-membered heterocyclyl optionally substituted by halogen, oxo, or C₁-C₆ alkyl optionally substituted by halogen;

R¹³ and R¹⁴ are each independently hydrogen, —OH, C₁-C₆ alkoxy, or C₁-C₆ alkyl, wherein the C₁-C₆ alkyl of R¹³ and R¹⁴ are optionally substituted by halogen, —OR, —NR¹⁵R¹⁶, or oxo,
or R¹³ and R¹⁴ are taken together with the atom to which they attached to form a 3- to 6-membered heterocyclyl optionally substituted by halogen, oxo or C₁-C₆ alkyl optionally substituted by halogen or oxo;

R¹⁵ and R¹⁶ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo,
or R¹⁵ and R¹⁶ are taken together with the atom to which they attached to form a 3- to 6-membered heterocyclyl optionally substituted by halogen, oxo or C₁-C₆ alkyl optionally substituted by oxo or halogen;

p and q are each independently 0, 1, 2, 3 or 4.

In some embodiments of a compound of Formula (I), or a salt thereof, the compound is other than the compounds in Table 1X, an isomer, or a salt thereof. In some embodiments of a compound of Formula (K), or a salt thereof, the compound is other than the compounds in Table 1X, an isomer, or a salt thereof.

TABLE 1X

| | |
|---|---|
| 1x | 4-((6-(difluoromethyl)-8-((1R,2R)-2-hydroxy-2-methylcyclopentyl)-7-oxo- |
| | 7,8-dihydropyrido[2,3-d]pyrimidin-2-yl)amino)piperidine-1-sulfonamide |
| 2x | 2-((4-cyanopyridin-2-yl)amino)-8-cyclopentyl-7-oxo-7,8-dihydropyrido[2,3- |
| | d]pyrimidine-6-carbonitrile |
| 3x | 2-((1H-indol-4-yl)amino)-8-cyclopentyl-7-oxo-7,8-dihydropyrido[2,3- |
| | d]pyrimidine-6-carbonitrile |
| 4x | 6-(difluoromethyl)-8-((1R,2S,3R)-3-hydroxy-2-methylcyclopentyl)-2-((1- |
| | (methylsulfonyl)piperidin-4-yl)amino)pyrido[2,3-d]pyrimidin-7(8H)-one |
| 5x | 6-(2,2-difluoroethyl)-8-((1R,2R)-2-hydroxy-2-methylcyclopentyl)-2-((1- |
| | (methylsulfonyl)piperidin-4-yl)amino)pyrido[2,3-d]pyrimidin-7(8H)-one |
| 6x | 2-((1H-indol-5-yl)amino)-8-cyclopentyl-7-oxo-7,8-dihydropyrido[2,3- |
| | d]pyrimidine-6-carbonitrile |
| 7x | 8-cyclopentyl-2-((4-(4-methylpiperazin-1-yl)phenyl)amino)-7-oxo-7,8- |
| | dihydropyrido[2,3-d]pyrimidine-6-carbonitrile |
| 8x | 8-cyclopentyl-7-oxo-2-(quinolin-3-ylamino)-7,8-dihydropyrido[2,3- |
| | d]pyrimidine-6-carbonitrile |
| 9x | 8-cyclopentyl-2-((4-morpholinophenyl)amino)-7-oxo-7,8-dihydropyrido[2,3- |
| | d]pyrimidine-6-carbonitrile |
| 10x | 6-(2,2-difluoroethyl)-8-((1R,2S,3R)-3-hydroxy-2-methylcyclopentyl)-2-((1- |
| | (methylsulfonyl)piperidin-4-yl)amino)pyrido[2,3-d]pyrimidin-7(8H)-one |
| 11x | 6-(difluoromethyl)-8-((1R,2R)-2-hydroxy-2-methylcyclopentyl)-2-((1- |
| | (methylsulfonyl)piperidin-4-yl)amino)pyrido[2,3-d]pyrimidin-7(8H)-one |
| 12x | 8-cyclopentyl-7-oxo-2-(quinolin-8-ylamino)-7,8-dihydropyrido[2,3- |
| | d]pyrimidine-6-carbonitrile |
| 13x | 8-cyclopentyl-2-((3,5-dimethoxyphenyl)amino)-7-oxo-7,8- |
| | dihydropyrido[2,3-d]pyrimidine-6-carbonitrile |
| 14x | 6-(difluoromethyl)-8-((1R,3R)-3-hydroxycyclohexyl)-2-((1- |
| | (methylsulfonyl)piperidin-4-yl)amino)pyrido[2,3-d]pyrimidin-7(8H)-one |
| 15x | 6-(difluoromethyl)-8-((1R,3R)-3-hydroxycyclopentyl)-2-((1- |
| | (methylsulfonyl)piperidin-4-yl)amino)pyrido[2,3-d]pyrimidin-7(8H)-one |
| 16x | 8-cyclopentyl-2-((2-methoxyquinolin-6-yl)amino)-7-oxo-7,8- |
| | dihydropyrido[2,3-d]pyrimidine-6-carbonitrile |
| 17x | 8-cyclopentyl-7-oxo-2-((3,4,5-trimethoxyphenyl)amino)-7,8- |
| | dihydropyrido[2,3-d]pyrimidine-6-carbonitrile |
| 18x | 1-cyclopentyl-3-(difluoromethyl)-4-methyl-7-((5-(piperazin-1-yl)pyrazin-2- |
| | yl)amino)-1,6-naphthyridin-2(1H)-one |
| 19x | 8-cyclopentyl-2-((5-morpholinopyridin-2-yl)amino)-7-oxo-7,8- |
| | dihydropyrido[2,3-d]pyrimidine-6-carbonitrile |
| 20x | 8-cyclohexyl-2-((4-(4-methylpiperazin-1-yl)phenyl)amino)-7-oxo-7,8- |
| | dihydropyrido[2,3-d]pyrimidine-6-carbonitrile |
| 21x | 1-cyclopentyl-3-(difluoromethyl)-4-methyl-7-((5-(piperazin-1-yl)pyrazin-2- |
| | yl)amino)-1,6-naphthyridin-2(1H)-one |
| 22x | 6-(2,2-difluoroethyl)-8-((1R,3R)-3-hydroxycyclohexyl)-2-((1- |
| | (methylsulfonyl)piperidin-4-yl)amino)pyrido[2,3-d]pyrimidin-7(8H)-one |
| 23x | 8-cyclopentyl-2-((5-(4-methylpiperazin-1-yl)pyridin-2-yl)amino)-7-oxo-7,8- |
| | dihydropyrido[2,3-d]pyrimidine-6-carbonitrile |
| 24x | 2-((4-chlorophenyl)amino)-8-cyclopentyl-7-oxo-7,8-dihydropyrido[2,3- |
| | d]pyrimidine-6-carbonitrile |
| 25x | 1-cyclopentyl-3-(difluoromethyl)-7-((5-(4-isopropylpiperazin-1-yl)pyrazin- |
| | 2-yl)amino)-4-methyl-1,6-naphthyridin-2(1H)-one |
| 26x | 8-cyclopentyl-6-(difluoromethyl)-2-((1-(methylsulfonyl)piperidin-4- |
| | yl)amino)pyrido[2,3-d]pyrimidin-7(8H)-one |
| 27x | 8-cyclopentyl-7-oxo-2-((4-(5-(trifluoromethyl)pyridin-2-yl)piperazin-1- |
| | yl)amino)-7,8-dihydropyrido[2,3-d]pyrimidine-6-carbonitrile |
| 28x | 2-((4-cyanophenyl)amino)-8-cyclopentyl-7-oxo-7,8-dihydropyrido[2,3- |
| | d]pyrimidine-6-carbonitrile |
| 29x | 8-cyclopentyl-2-((2-hydroxyphenyl)amino)-7-oxo-7,8-dihydropyrido[2,3- |
| | d]pyrimidine-6-carbonitrile |
| 30x | 5-amino-8-cyclobutyl-2-(cyclohexylamino)-7-oxo-7,8-dihydropyrido[2,3- |
| | d]pyrimidine-6-carbonitrile |
| 31x | tert-butyl 4-(5-((1-cyclopentyl-3-(difluoromethyl)-4-methyl-2-oxo-1,2- |
| | dihydro-1,6-naphthyridin-7-yl)amino)pyrazin-2-yl)piperazine-1-carboxylate |
| 32x | 4-((6-(difluoromethyl)-8-((1R,2R)-2-hydroxy-2-methylcyclopentyl)-7-oxo- |
| | 7,8-dihydropyrido[2,3-d]pyrimidin-2-yl)amino)-N-methylpiperidine-1- |
| | sulfonamide |
| 33x | 8-methyl-2-((4-(4-methylpiperazin-1-yl)phenyl)amino)-7-oxo-7,8- |
| | dihydropyrido[2,3-d]pyrimidine-6-carbonitrile |
| 34x | 8-cyclopentyl-2-((2-methoxyphenyl)amino)-7-oxo-7,8-dihydropyrido[2,3- |
| | d[pyrimidine-6-carbonitrile |
| 35x | 8-cyclopentyl-2-((4-(5-morpholino-7-oxo-7H-thieno[3,2-b]pyran-3- |
| | yl)phenyl)amino)-7-oxo-7,8-dihydropyrido[2,3-d]pyrimidine-6-carbonitrile |
| 36x | 8-ethyl-2-((4-(4-methylpiperazin-1-yl)phenyl)amino)-7-oxo-7,8- |
| | dihydropyrido[2,3-d]pyrimidine-6-carbonitrile |
| 37x | 8-cyclopentyl-2-((3-methoxyphenyl)amino)-7-oxo-7,8-dihydropyrido[2,3- |
| | d]pyrimidine-6-carbonitrile |
| 38x | 8-((4-cyclopropylpyridin-3-yl)methyl)-2-((3-fluoro-4-(piperazin-1- |
| | yl)phenyl)amino)-7-oxo-7,8-dihydropyrido[2,3-d]pyrimidine-6-carbonitrile |
| 39x | 8-((1R,2R)-2-hydroxy-2-methylcyclopentyl)-2-((1- |
| | (methylsulfonyl)piperidin-4-yl)amino)-6-(trifluoromethyl)pyrido[2,3- |
| | d]pyrimidin-7(8H)-one |
| 40x | 2-((4-(4-methylpiperazin-1-yl)phenyl)amino)-7-oxo-8-propyl-7,8- |
| | dihydropyrido[2,3-d]pyrimidine-6-carbonitrile |
| 41x | 8-cyclopentyl-2-((4-methoxyphenyl)amino)-7-oxo-7,8-dihydropyrido[2,3- |
| | d]pyrimidine-6-carbonitrile |
| 42x | 8-((3-cyclopropylpyridin-4-yl)methyl)-2-((3-fluoro-4-(piperazin-1- |
| | yl)phenyl)amino)-7-oxo-7,8-dihydropyrido[2,3-d]pyrimidine-6-carbonitrile |
| 43x | 6-(2,2-difluoroethyl)-8-((1R,3R)-3-hydroxycyclopentyl)-2-((1- |
| | (methylsulfonyl)piperidin-4-yl)amino)pyrido[2,3-d]pyrimidin-7(8H)-one |
| 44x | 8-cyclopentyl-2-((1-(methylsulfonyl)piperidin-4-yl)amino)-6- |
| | (trifluoromethyl)pyrido[2,3-d]pyrimidin-7(8H)-one |
| 45x | 8-isopropyl-2-((4-(4-methylpiperazin-1-yl)phenyl)amino)-7-oxo-7,8- |
| | dihydropyrido[2,3-d]pyrimidine-6-carbonitrile |
| 46x | 8-cyclopentyl-2-((2,4-dimethoxyphenyl)amino)-7-oxo-7,8- |
| | dihydropyrido[2,3-d]pyrimidine-6-carbonitrile |
| 47x | 8-((4-cyclopropylpyrimidin-5-yl)methyl)-2-((3-fluoro-4-(piperazin-1- |
| | yl)phenyl)amino)-7-oxo-7,8-dihydropyrido[2,3-d]pyrimidine-6-carbonitrile |
| 48x | 8-cyclopentyl-6-(2,2-difluoroethyl)-2-((1-(methylsulfonyl)piperidin-4- |
| | yl)amino)pyrido[2,3-d]pyrimidin-7(8H)-one |
| 49x | 8-butyl-2-((4-(4-methylpiperazin-1-yl)phenyl)amino)-7-oxo-7,8- |
| | dihydropyrido[2,3-d]pyrimidine-6-carbonitrile |
| 50x | 8-cyclopentyl-2-((3,4-dimethoxyphenyl)amino)-7-oxo-7,8- |
| | dihydropyrido[2,3-d]pyrimidine-6-carbonitrile |
| 51x | 8-((3-cyclopropylpyridin-2-yl)methyl)-2-((3-fluoro-4-(piperazin-1- |
| | yl)phenyl)amino)-7-oxo-7,8-dihydropyrido[2,3-d]pyrimidine-6-carbonitrile |
| 52x | 2-((4-(4-methylpiperazin-1-yl)phenyl)amino)-7-oxo-8-pentyl-7,8- |
| | dihydropyrido[2,3-d]pyrimidine-6-carbonitrile |
| 53x | 8-cyclopentyl-2-((5-fluoro-2-methoxyphenyl)amino)-7-oxo-7,8- |
| | dihydropyrido[2,3-d]pyrimidine-6-carbonitrile |
| 54x | 6-(difluoromethyl)-8-((1S,2S)-2-hydroxy-2-methylcyclopentyl)-2-((1- |
| | (methylsulfonyl)piperidin-4-yl)amino)pyrido[2,3-d]pyrimidin-7(8H)-one |
| 55x | 8-cyclopropyl-2-((4-(4-methylpiperazin-1-yl)phenyl)amino)-7-oxo-7,8- |
| | dihydropyrido[2,3-d]pyrimidine-6-carbonitrile |
| 56x | 8-cyclopentyl-7-oxo-2-(pyridin-2-ylamino)-7,8-dihydropyrido[2,3- |
| | d]pyrimidine-6-carbonitrile |
| 57x | 6-(chlorodifluoromethyl)-8-((1R,3R)-3-hydroxy-3-methylcyclopentyl)-2-((1- |
| | (methylsulfonyl)piperidin-4-yl)amino)pyrido[2,3-d]pyrimidin-7(8H)-one |
| 58x | 6-(bromodifluoromethyl)-8-((1R,3R)-3-hydroxy-3-methylcyclopentyl)-2- |
| | ((1-(methylsulfonyl)piperidin-4-yl)amino)pyrido[2,3-d]pyrimidin-7(8H)-one |
| 59x | 6-(difluoroiodomethyl)-8-((1R,3R)-3-hydroxy-3-methylcyclopentyl)-2-((1- |
| | (methylsulfonyl)piperidin-4-yl)amino)pyrido[2,3-d]pyrimidin-7(8H)-one |
| 60x | tert-butyl (3-((6-cyano-2-((1-(2-methoxyethyl)-1H-pyrazol-4-yl)amino)-7- |
| | oxopyrido[2,3-d]pyrimidin-8(7H)-yl)methyl)phenyl)carbamate |
| 61x | tert-butyl 4-(6-((6-cyano-8-cyclopentyl-5-methyl-7-oxo-7,8- |
| | dihydropyrido[2,3-d]pyrimidin-2-yl)amino)pyridin-3-yl)piperazine-1- |
| | carboxylate |
| 62x | 8-(3-aminobenzyl)-2-((1-(2-methoxyethyl)-1H-pyrazol-4-yl)amino)-7-oxo- |
| | 7,8-dihydropyrido[2,3-d]pyrimidine-6-carbonitrile |
| 63x | 2-((5-amino-4-((2-(dimethylamino)ethyl)(methyl)amino)-2- |
| | methoxyphenyl)amino)-8-methyl-7-oxo-7,8-dihydropyrido[2,3- |
| | d]pyrimidine-6-carbonitrile |
| 64x | tert-butyl (3-(2-((4-(4-acetylpiperazin-1-yl)-2-methoxyphenyl)amino)-6- |
| | cyano-7-oxopyrido[2,3-d]pyrimidin-8(7H)-yl)phenyl)carbamate |
| 65x | tert-butyl 3-((4-((8-(3-aminobenzyl)-6-cyano-7-oxo-7,8-dihydropyrido[2,3- |
| | d]pyrimidin-2-yl)amino)-1H-pyrazol-1-yl)methyl)piperidine-1-carboxylate |
| 66x | 3-cyclohexyl-1-ethyl-7-((5-methylpyridin-2-yl)amino)-1,6-naphthyridin- |
| | 2(1H)-one |
| 67x | tert-butyl 4-(4-((6-cyano-8-cyclopentyl-7-oxo-7,8-dihydropyrido[2,3- |
| | d]pyrimidin-2-yl)amino)phenyl)piperazine-1-carboxylate |
| 68x | 8-cyclopentyl-7-oxo-2-((4-(piperazin-1-yl)phenyl)amino)-7,8- |
| | dihydropyrido[2,3-d]pyrimidine-6-carbonitrile |
| 69x | 3-cyclopropyl-1-ethyl-7-((5-methylpyridin-2-yl)amino)-1,6-naphthyridin- |
| | 2(1H)-one |
| 70x | 3-cyclopentyl-1-ethyl-7-((5-methylpyridin-2-yl)amino)-1,6-naphthyridin- |
| | 2(1H)-one |
| 71x | 3-cyclobutyl-1-ethyl-7-((5-methylpyridin-2-yl)amino)-1,6-naphthyridin- |
| | 2(1H)-one |

In some embodiments of a compound of Formula (J), when R is C₁-C₆ haloalkyl, then B is not hydrogen. In some embodiments of a compound of Formula (K) or any related formulae where applicable, when R is C₁-C₆ haloalkyl, then B is not hydrogen.

In some embodiments of a compound of Formula (K) or any related formulae, where applicable, when Q is O, R¹ is unsubstituted C₁-C₆ alkyl, R², R³, and R⁴ are H, W is

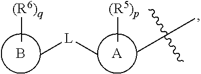

and A is phenyl or pyridyl, then
B is C₃-C₆ cycloalkyl, 5- to 7-membered heteroaryl, or phenyl, wherein the C₃-C₆ cycloalkyl, 5- to 7-membered heteroaryl, and phenyl of B are optionally substituted by R⁶.

In some embodiments of Formula (K) or any related formulae, where applicable, when Q is O, R¹ is unsubstituted C₁-C₆ alkyl, R², R³, and R⁴ are H, W is

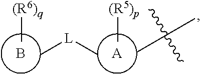

and A is phenyl or pyridyl, then
R is C₁-C₆ haloalkyl.

In some embodiments of Formula (K) or any related formulae, where applicable, when X and Y are N, Q is O, R is —CN, R¹ is unsubstituted cycloalkyl, R², R³, and R⁴ are H, W is

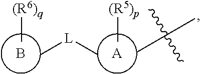

A is phenyl, and L is a bond, then
B is hydrogen, C₃-C₆ cycloalkyl, 5- to 7-membered heteroaryl, or phenyl, wherein the
C₃-C₆ cycloalkyl, 5- to 7-membered heteroaryl, and phenyl of B are optionally substituted by R⁶.

In some embodiments of Formula (K) or any related formulae, where applicable, when X and Y are N, Q is O, R is —CN, R¹ is unsubstituted cycloalkyl, R², R³, and R⁴ are H, W is

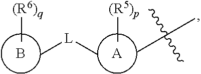

A is phenyl, and L is a bond, then
B is hydrogen, C₃-C₆ cycloalkyl, 3- to 10-membered heterocyclyl wherein the heterocyclyl is other than N-methyl piperazine, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶.

In some embodiments of Formula (K) or any related formulae, where applicable, when X and Y are N, Q is O, R¹ is C₁-C₆ haloalkyl, R¹ is cyclopentyl or cyclohexyl, R², R³ and R⁴ are H, W is

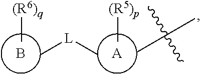

and A is heterocyclyl, then
B is C₃-C₆ cycloalkyl, 3- to 10-membered heterocyclyl, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶.

In some embodiments of Formula (K) or any related formulae, where applicable, when X and Y are N, Q is O, R is —CN, R¹ is cyclopentyl, R², R³, R⁴ are H, W is

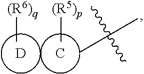

C is phenyl, and D is a 5-membered heteroaryl, then p is 1, 2, 3, or 4, or q is 1, 2, or 3. In some embodiments, when W is

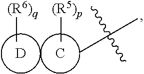

C is phenyl, and D is pyridyl, then q is 2, 3 or 4. In some embodiments, when X and Y are N, Q is O, R is —CN, R¹ is cyclopentyl, R², R³, R⁴ are H, W is

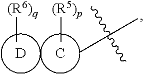

C is 6-membered heteroaryl and D is phenyl, then p is 1, 2, or 3 or q is 1, 2, 3, or 4.

In some embodiments of Formula (K) or any related formulae, where applicable, when X and Y are N, Q is O, R is —CN, R¹ is cyclopenyl, R², R³, and R⁴ are H, W is

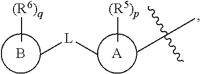

L is a bond, and A is phenyl or pyridyl, then
B is C₃-C₆ cycloalkyl, 3- to 10-membered heterocyclyl, 5- to 7-membered heteroaryl, or phenyl, or
p is at least 1 and at least one R⁵ is independently C₁-C₆ alkyl, C₂-C₆ alkenyl, C₂-C₆ alkynyl, C₁-C₆ haloalkyl, oxo, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰,
—(C₁-C₃ alkylene)SR¹⁰, —(C₁-C₃ alkylene)NR¹¹R¹², —(C₁-C₃ alkylene)C(O)R¹⁰,
—(C₁-C₃ alkylene)C(O)NR¹¹R¹², —(C₁-C₃ alkylene)NR¹⁰C(O)R¹¹, —(C₁-C₃
alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen.

In some embodiments of Formula (K) or any related formulae, where applicable, when Q is O, R —CN or C₁-C₆ haloalkyl, R¹ is cyclopentyl, R² is —CH₃, R³ and R⁴ are H, W is

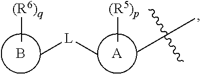

and A is 5- to 7-membered heteroaryl then
B is H, C₃-C₆ cycloalkyl, 5- to 7-membered heteroaryl, or phenyl.

In some embodiments of Formula (K) or any related formulae, where applicable, when X and Y are N, Q is O, R is —CN, R¹ is cyclopentyl, R², R³, and R⁴ are H, W is

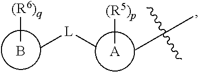

L is a bond, A is phenyl or pyridyl, and p is 0, then B is

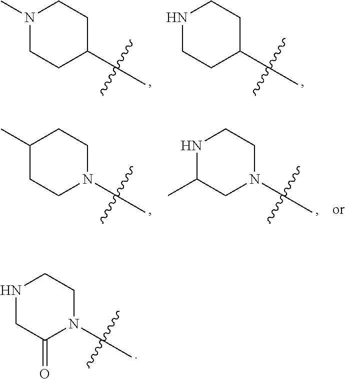

In some embodiments, when X and Y are N, Q is O, R is —CN, R¹ is cyclopentyl, R², R³, and R⁴ are H, W is

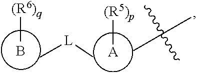

L is a bond, and A is phenyl or pyridyl, and p is 0, then B is not N

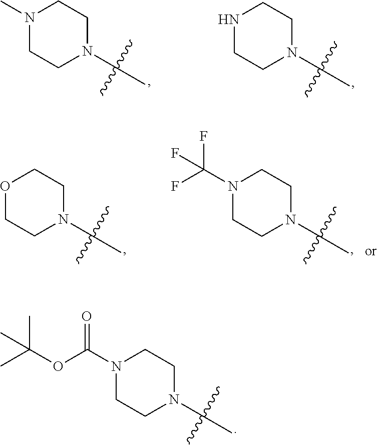

In some embodiments of Formula (K) or any related formulae, where applicable, when X and Y are N, Q is O, R is —CN, R¹ is cyclopentyl, W is

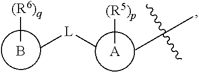

A is phenyl, L is a bond, and B is a 9-membered heteroaryl, then
p is 1, 2, 3, or 4. In some embodiments, when X and Y are N, Q is O, R is —CN, R¹ is cyclopentyl, W is

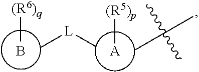

A is phenyl, L is a bond, and B is a 9-membered heteroaryl, then B comprises 2 annular N atoms.

In some embodiments of Formula (K) or any related formulae, where applicable, when X and Y are N, Q is O, R is —CN, R², R³, and R⁴ are H, W is

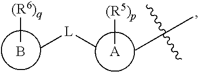

A is phenyl, p is 1, R⁵ is —OR¹⁰, L is a bond, B is 3- to 10-membered heterocyclyl, and R¹ is phenyl, then
R¹ is optionally substituted by halogen, oxo, —OR¹³, —C(O)R¹³, —CN, C₃-C₈ cycloalkyl, or C₁-C₆ alkyl optionally substituted by oxo, —OH or halogen.
In some embodiments of Formula (K), when X and Y are N, Q is O, R is —CN, R², R³, and R⁴ are H, W is

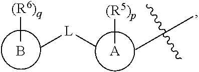

A is phenyl, L is a bond, and B is 3- to 10-membered heterocyclyl, R¹ is phenyl substituted by —NR¹³R¹⁴, and p is 1, then
R⁵ is C₁-C₆ alkyl, C₂-C₆ alkenyl, C₂-C₆ alkynyl, C₁-C₆ haloalkyl, halogen, oxo, —CN, —SR¹⁰, —NR¹¹R¹², —C(O)R¹⁰, —C(O)NR¹¹R¹², —OC(O)NR¹¹R¹², —NR¹⁰C(O)R¹¹,
—NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰,
—(C₁-C₃ alkylene)NR¹¹R¹², —(C₁-C₃ alkylene)C(O)R¹⁰, —(C₁-C₃ alkylene)C(O)NR¹¹R¹²,
—(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹²,
—(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³,
—(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen.

In some embodiments of Formula (K) or any related formulae, where applicable, when X and Y are N, Q is O, R is —CN, R², R³, and R⁴ are H, W is

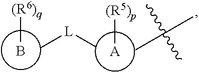

R¹ is —(C₁-C₃ alkylene)(C₆-C₁₄ aryl), and A is pyrazolyl, then
R¹ is optionally substituted by halogen, oxo, —OR¹³, —C(O)R¹³, —CN, C₃-C₈ cycloalkyl, or C₁-C₆ alkyl optionally substituted by oxo, —OH or halogen.

In some embodiments of Formula (K) or any related formulae, where applicable, when X and Y are N, Q is O, R is —CN, R², R³, and R⁴ are H, W is

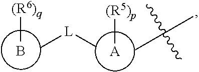

R¹ is —(C₁-C₃ alkylene)(3- to 12-membered heterocyclyl) substituted by cycloalkyl, A is phenyl, and p is 1, then
R⁵ is C₁-C₆ alkyl, C₂-C₆ alkenyl, C₂-C₆ alkynyl, C₁-C₆ haloalkyl, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹,
—NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen.

In some embodiments of Formula (K) or any related formulae, where applicable, when X and Y are N, Q is O, R is —CN, R², R³, and R⁴ are H, W is

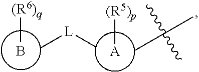

A is phenyl, p is 1, and R⁵ is fluoro, then
R¹ is C₁-C₆ alkyl, C₃-C₈ cycloalkyl, 3- to 12-membered heterocyclyl, 5- to 10-membered heteroaryl, C₆-C₁₄ aryl, —(C₁-C₃ alkylene)(C₃-C₆ cycloalkyl), —(C₁-C₃ alkylene)(3- to 12-membered heterocyclyl), —C(O)R¹⁰, —(C₁-C₃ alkylene)(5- to 10-membered heteroaryl), or —(C₁-C₃ alkylene)(C₆-C₁₄ aryl), each of which is independently optionally substituted by halogen, oxo, —OR¹³, —NR¹³R¹⁴, —C(O)R¹³, —CN, or C₁-C₆ alkyl optionally substituted by oxo, —OH or halogen.

In some embodiments of a compound of Formula (J), W is

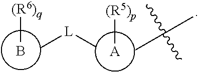

In some embodiments, W is

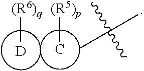

In some embodiments of a compound of Formula (K) or any related formulae where applicable, W is as provided herein to the same extent as is described for Formula (J).

In some embodiments of a compound of Formula (J), W is

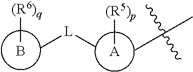

and provided is a compound of Formula (I):

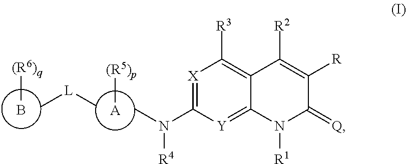

or a salt thereof, wherein X, Y, R, Q, A, B, L, R¹, R², R³, R⁴, R⁵, R⁶, p, and q are as detailed herein for Formula (J). In some embodiments of a compound of Formula (K) or any related formulae where applicable, Formula (I) is as provided herein to the same extent as is described for Formula (J).

In some embodiments of a compound of Formula (J), W is

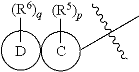

and provided is a compound of Formula (II):

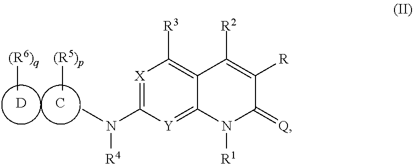

or a salt thereof, wherein X, Y, R, Q, C, D, R¹, R², R³, R⁴, R⁵, R⁶, p, and q are as detailed herein for Formula (J). In some embodiments of a compound of Formula (K) or any related formulae where applicable, Formula (II) is as provided herein to the same extent as is described for Formula (J).

Specific values listed below are values for a compound of Formula (J) as well as all related formulae such as Formula (I), (I-B1) to (I-B22), (I-C1) to (I-C23), and Formula (II), where applicable. It is to be understood that two or more values may combined. Thus, it is to be understood that any variable for a compound of Formula (J) as well as all related formulae such as Formula (I), (I-B1) to (I-B22), (I-C1) to (I-C23), and Formula (II) the same as if each and every combination of variables were specifically and individually listed. For example, it is understood that any specific value of R¹ detailed herein for a compound of Formula (J) as well as all related formulae such as Formula (I), (I-B1) to (I-B22), (I-C1) to (I-C23), and Formula (II) may be combined with any other specific value for one or more of the variables X, Y, R, Q, A, B, C, D, R², R³, R⁴, R⁵, R⁶, L, p, and q the same as if each and every combination were specifically and individually listed. In some embodiments, the values listed for Formula (J) are equally applicable to a compound of Formula (K) as well as all related formulae such as Formula (J), (I), (I-B1) to (I-B22), (I-C1) to (I-C23), and Formula (II), where applicable. Likewise, it is to be understood that any variable for a compound of Formula (K) and any related formulae, where applicable, may be combined with any other variable or combination of variables as though every combination of variables were specifically and individually listed.

In some embodiments, provided is a compound of any one of Formula (I-B1) to (I-B22), or a salt thereof:

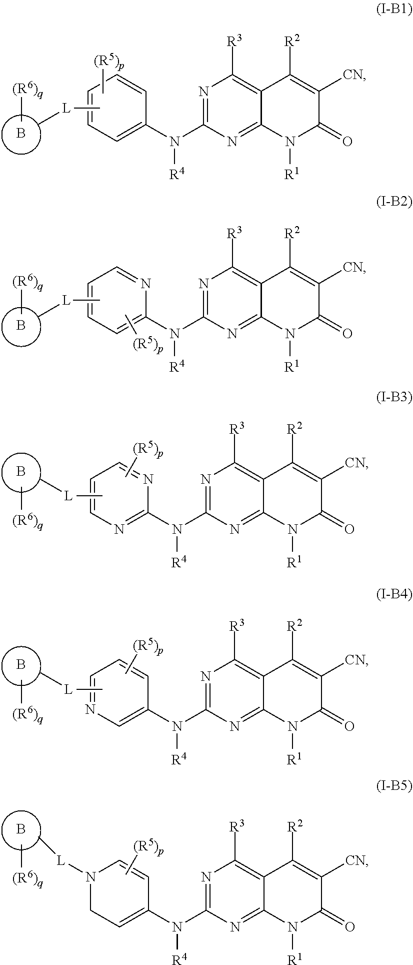

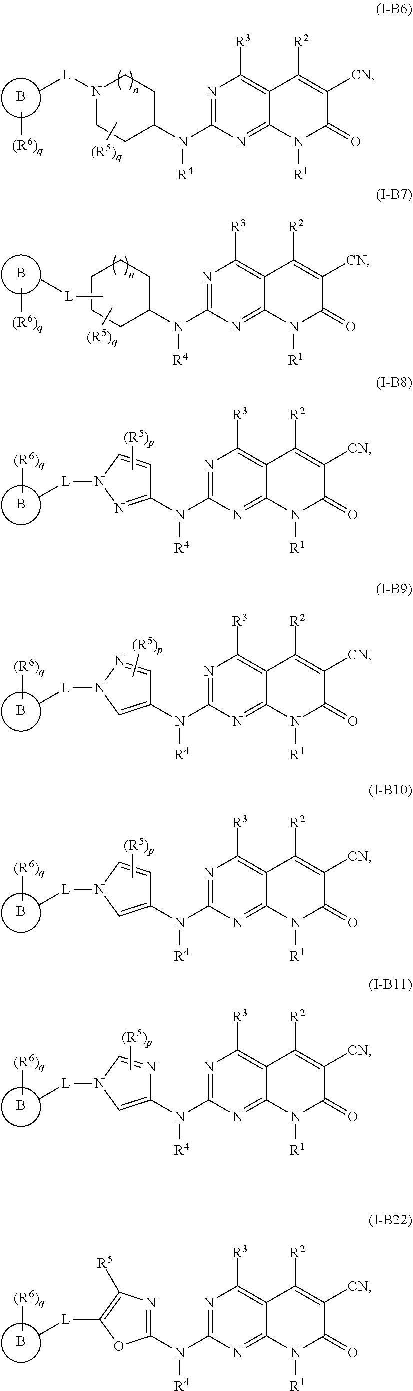

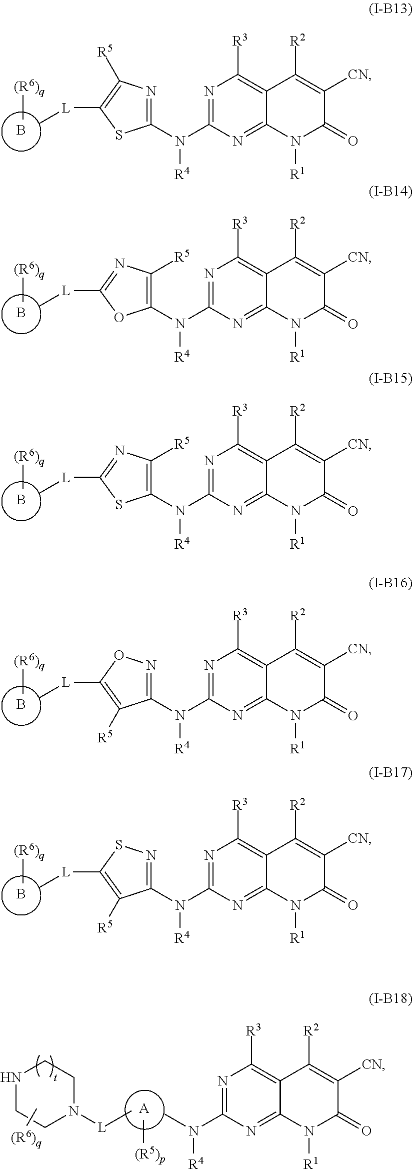

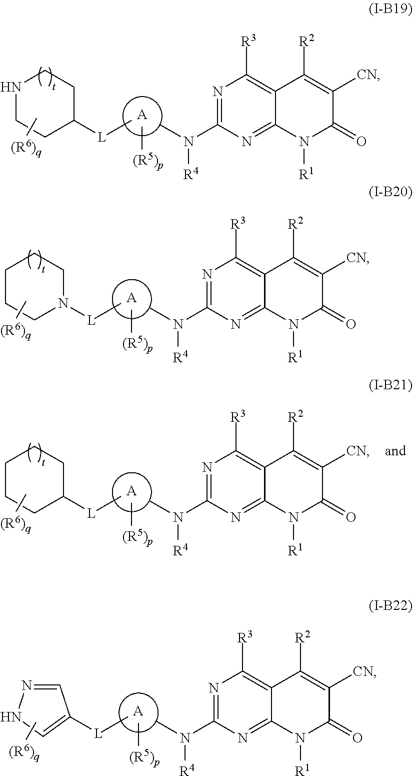

wherein R, Q, A, B, L, R¹, R², R³, R⁴, R⁵, R⁶, p, and q are as described herein for Formula (J); and t is 0, 1, 2 or 3. In some embodiments, t is 0. In some embodiments, t is 1. In some embodiments, t is 2. In some embodiments, t is 0 or 1. In some embodiments, t is 0, 1, or 2. In some embodiments, a compound of Formula (J) is a compound of Formula (I-B1). In some embodiments, a compound of Formula (J) is a compound of Formula (I-B2). In some embodiments, a compound of Formula (J) is a compound of Formula (I-B3). In some embodiments, a compound of Formula (J) is a compound of Formula (I-B4). In some embodiments, a compound of Formula (J) is a compound of Formula (I-B5). In some embodiments, a compound of Formula (J) is a compound of Formula (I-B6). In some embodiments, a compound of Formula (J) is a compound of Formula (I-B7). In some embodiments, a compound of Formula (J) is a compound of Formula (I-B8). In some embodiments, a compound of Formula (J) is a compound of Formula (I-B9). In some embodiments, a compound of Formula (J) is a compound of Formula (I-B10). In some embodiments, a compound of Formula (J) is a compound of Formula (I-B11). In some embodiments, a compound of Formula (J) is a compound of Formula (I-B12). In some embodiments, a compound of Formula (J) is a compound of Formula (I-B13). In some embodiments, a compound of Formula (J) is a compound of Formula (I-B14). In some embodiments, a compound of Formula (J) is a compound of Formula (I-B15). In some embodiments, a compound of Formula (J) is a compound of Formula (I-B16). In some embodiments, a compound of Formula (J) is a compound of Formula (I-B17). In some embodiments, a compound of Formula (J) is a compound of Formula (I-B18). In some embodiments, a compound of Formula (J) is a compound of Formula (I-B19). In some embodiments, a compound of Formula (J) is a compound of Formula (I-B20). In some embodiments, a compound of Formula (J) is a compound of Formula (I-B21). In some embodiments, a compound of Formula (J) is a compound of Formula (I-B22). In some embodiments, a compound of Formula (I-B6) has the Formula (I-B6.1)

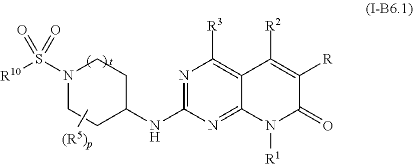

wherein R₁═C₃-C₁₂ cycloalkyl optionally substituted with 1-3 groups independently selected from the group consisting of halogen, C₁-C₃ alkyl, hydroxyl, cyano, and —OCH₃; R═CN, CH₂F, CHF₂ or CF₃; R²═H or CH₃; R₃═H or F; and R¹⁰═C₁-C₆ alkyl or 5-6 member heteroaryl optionally substituted with halogen or C₁-C₃ alkyl. In some embodiments, a compound of Formula of (I-B6) is a compound of Formula (I-B6.2)

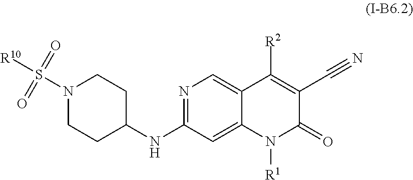

or a salt thereof, wherein R¹═C₃-C₁₂ cycloalkyl optionally substituted with 1-3 groups independently selected from the group consisting of halogen, C₁-C₃ alkyl, hydroxyl, cyano, and —OCH₃; R²═CH₃ or H; and R¹⁰ is C₁-C₃ alkyl or 5-6 membered heteroaryl optionally substituted with from 1-2 C₁-C₃ alkyl, halogen or cyano. In some embodiments, a compound of Formula (I-B6) is a compound of Formula (I-B6.3)

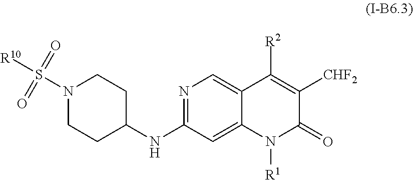

or a salt thereof, wherein R¹═C₃-C₁₂ cycloalkyl optionally substituted with 1-3 groups independently selected from the group consisting of halogen, C₁-C₃ alkyl, hydroxyl, cyano, and —OCH₃; R²═CH₃ or H; and R¹⁰ is C₁-C₃ alkyl or 5-6 membered heteroaryl optionally substituted with from 1-2 C₁-C₃ alkyl, halogen or cyano. In some embodiments of Formula (I-B6), including Formula (I-B6.1), (I-B6.2), and (I-B6.3), R¹═C₃-C₈ cycloalkyl optionally substituted with 1-3 groups independently selected from the group consisting of halogen, C₁-C₃ alkyl, hydroxyl, cyano, and —OCH₃. In some embodiments of Formula (I-B6), including Formula (I-B6.1), (I-B6.2), and (I-B6.3),

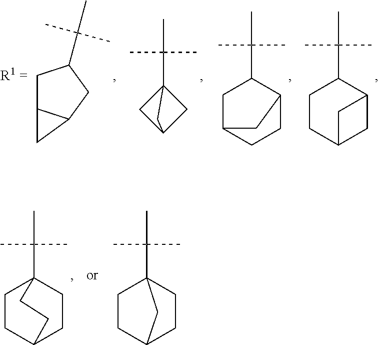

optionally substituted with 1-3 groups independently selected from the group consisting of halogen, C₁-C₃ alkyl, hydroxyl, cyano, and —OCH₃.

In some embodiments of a compound of Formula (J), A is C₃-C₆ cycloalkyl, 4- to 7-membered heterocyclyl, 5- to 7-membered heteroaryl or phenyl, each of which is unsubstituted. In some embodiments of a compound of Formula (I), A is C₃-C₆ cycloalkyl, 4- to 7-membered heterocyclyl, 5- to 7-membered heteroaryl or phenyl, each of which is independently optionally substituted by R⁵. In some embodiments of a compound of Formula (I), A is phenyl optionally substituted by R⁵. In some embodiments of a compound of Formula (I), A is 5- to 7-membered heteroaryl optionally substituted by R⁵. In some embodiments of a compound of Formula (I), A is selected from the group consisting of pyridinyl, pyrimidinyl, pyrazolyl, pyrrolyl, thiazolyl, oxazolyl, or imidazolyl, each of which is independently optionally substituted by R⁵. In some embodiments of a compound of Formula (I), A is 4- to 7-membered heterocyclyl, optionally substituted by R⁵. In some embodiments of a compound of Formula (I), A is piperidinyl, pyrrolidinyl, azetidinyl, dihydropyridinyl, or pyridone, each of optionally substituted by R⁵. In some embodiments of a compound of Formula (I), A is C₃-C₆ cycloalkyl substituted by R⁵. In some embodiments A is cyclohexyl or cyclopentyl, each of optionally substituted by R⁵. In some embodiments of a compound of Formula (I), A is phenyl, pyridinyl, pyrimidinyl, pyrazolyl, thiazolyl, oxazolyl, isooxazolyl, imidazolyl, piperidinyl, pyrrolidinyl, azetidinyl, pyridone, cyclohexyl, or cyclopentyl, each of which is unsubstituted. In some embodiments of a compound of Formula (I), A is phenyl, pyridinyl, pyrimidinyl, pyrazolyl, thiazolyl, oxazolyl, isooxazolyl, imidazolyl, piperidinyl, pyrrolidinyl, azetidinyl, dihydropyridinyl, pyridone, cyclohexyl, or cyclopentyl, each of which is independently optionally substituted by R⁵. In some embodiments of a compound of Formula (K) or any related formulae where applicable, A is as provided herein to the same extent as is described for Formula (J).

In some embodiments of a compound of Formula (J), B is hydrogen, C₃-C₆ cycloalkyl, 3- to 10-membered heterocyclyl, 5- to 7-membered heteroaryl, or phenyl, each of which is independently optionally substituted by R⁶. In some embodiments of a compound of Formula (I), B is C₃-C₆ cycloalkyl, 3- to 10-membered heterocyclyl, 5- to 7-membered heteroaryl, or phenyl, each of which is independently optionally substituted by R⁶. In some embodiments of a compound of Formula (I), B is C₃-C₆ cycloalkyl, 3- to 10-membered heterocyclyl, 5- to 7-membered heteroaryl, or phenyl, each of which is unsubstituted. In some embodiments of a compound of Formula (I), B is hydrogen. In some embodiments of a compound of Formula (I), B is 3- to 10-membered heterocyclyl optionally substituted by R⁶. In some embodiments of a compound of Formula (I), B is diazepanyl, azepanyl, piperazinyl, piperidinyl, morpholinyl, pyrrolidinyl or azetidinyl, each of which is independently optionally substituted by R⁶. In some embodiments of a compound of Formula (I), B is 5- to 7-membered heteroaryl optionally substituted by R⁶. In some embodiments of a compound of Formula (I), B is imidazolyl or pyrazolyl, each of which is independently optionally substituted by R⁶. In some embodiments of a compound of Formula (I), B is phenyl optionally substituted by R⁶. In some embodiments of a compound of Formula (I), B is C₃-C₆ cycloalkyl optionally substituted by R⁶. In some embodiments of a compound of Formula (I), B is cyclopentyl, cyclohexyl, or cycloheptyl, each of which is independently optionally substituted by R⁶. In some embodiments of a compound of Formula (I), B is diazepanyl, azepanyl, piperazinyl, piperidinyl, morpholinyl, pyrrolidinyl, azetidinyl, imidazolyl, pyrazolyl, phenyl, cyclopentyl, cyclohexyl, or cycloheptyl, each of which is unsubstituted. In some embodiments of a compound of Formula (I), B is diazepanyl, azepanyl, piperazinyl, piperidinyl, pyrrolidinyl, azetidinyl, imidazolyl, pyrazolyl, phenyl, cyclopentyl, cyclohexyl, or cycloheptyl, each of which is independently optionally substituted by R⁶. In some embodiments of a compound of Formula (K) or any related formulae where applicable, B is as provided herein to the same extent as is described for Formula (J).

In some embodiments of a compound of Formula (I), L is a bond, —CH₂—, —NH—, —O—, —S—, —S(O)₂—, —C(O)—, —NCH₃—, —S(O)₂NH—, or —NHS(O)₂—. In some embodiments of a compound of Formula (I), L is a bond, —CH₂—, —NH—, —O—, or —S—. In some embodiments, L is a bond. In some embodiments, L is —CH₂—. In some embodiments, L is —NH—. In some embodiments, L is —S—. In some embodiments, L is —O—. In some embodiments, L is —S(O)₂—. In some embodiments, L is —C(O)—. In some embodiments, L is —NCH₃—. In some embodiments, L is —NHS(O)₂—. In some embodiments, L is —CR¹¹R¹²—. In some embodiments, L is —NR¹⁰—. In some embodiments, L is —NR¹⁰S(O)₂—. In some embodiments, L is —S(O)₂NR¹⁰—. In some embodiments, L is —SO₂NH—. In some embodiments of a compound of Formula (K) or any related formulae where applicable, L is as provided herein to the same extent as is described for Formula (J).

In some embodiments, provided is a compound of any one of Formula (I-C1) to (I-C23):

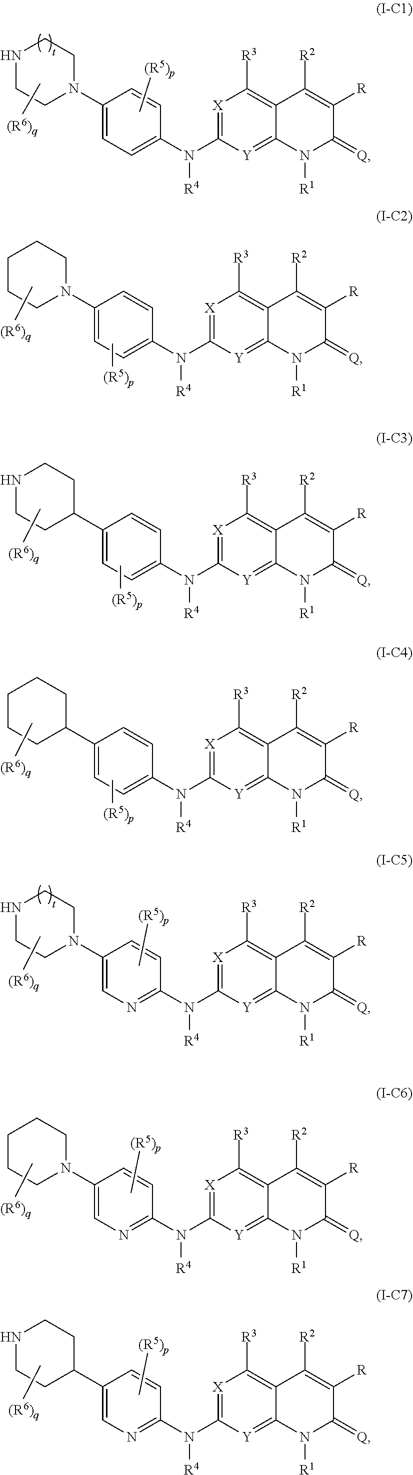

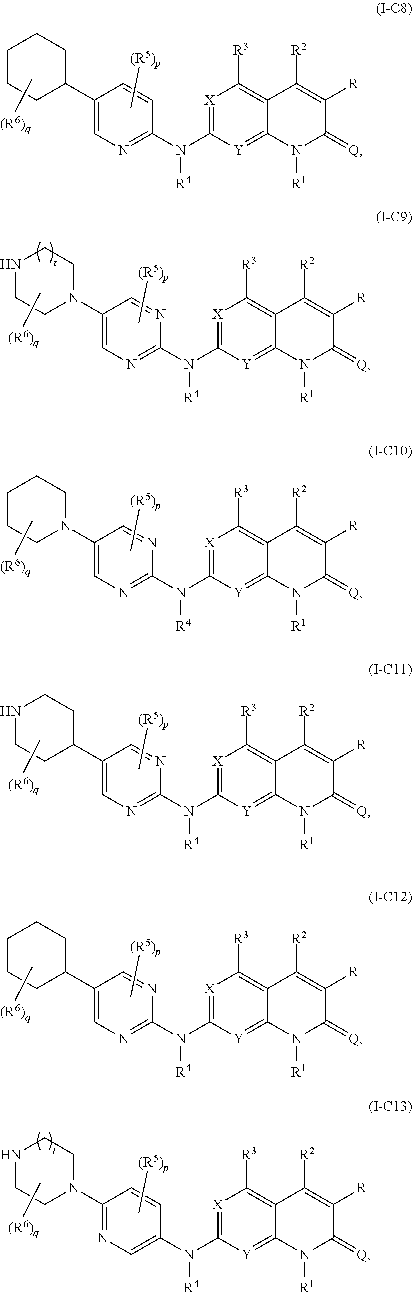

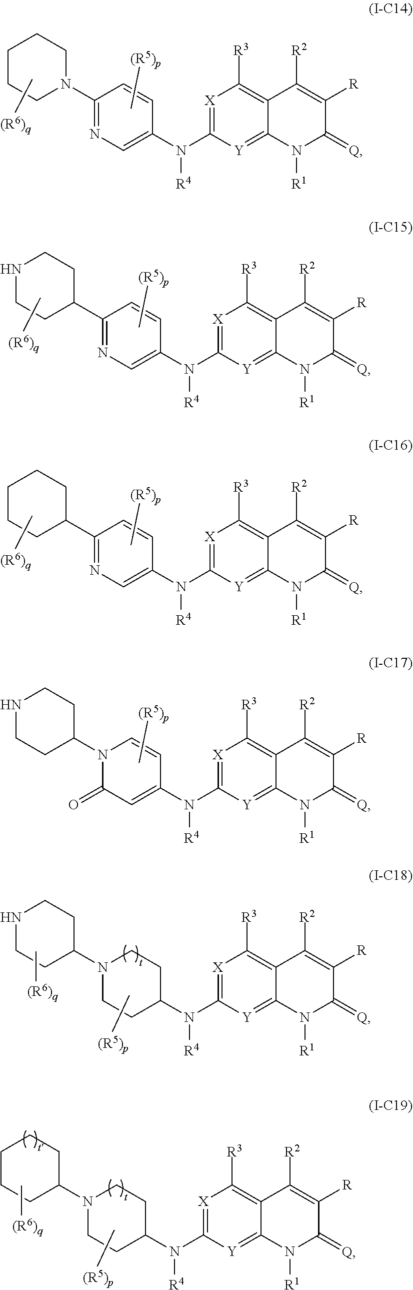

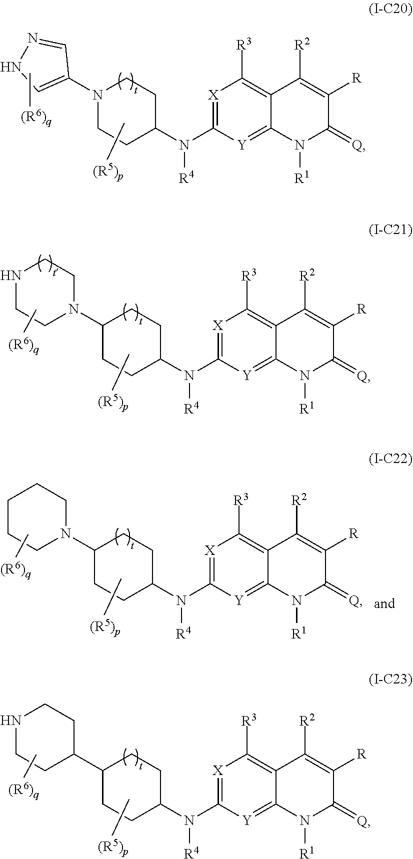

or a salt thereof, wherein R, Q, R¹, R², R³, R⁴, R⁵, R⁶, p, and q are as described herein for Formula (J); t and t′ are each independently 0, 1, 2, or 3. In some embodiments, t is 0. In some embodiments, t is 0 or 1. In some embodiments, t is 0, 1, or 2. In some embodiments, t′ is 0. In some embodiments, t′ is 0 or 1. In some embodiments, t′ is 0, 1, or 2. In some embodiments, a compound of Formula (J) is of Formula (I-C1). In some embodiments, a compound of Formula (J) is of Formula (I-C2). In some embodiments, a compound of Formula (J) is of Formula (I-C3). In some embodiments, a compound of Formula (J) is of Formula (I-C4). In some embodiments, a compound of Formula (J) is of Formula (I-C5). In some embodiments, a compound of Formula (J) is of Formula (I-C6). In some embodiments, a compound of Formula (J) is of Formula (I-C7). In some embodiments, a compound of Formula (J) is of Formula (I-C8). In some embodiments, a compound of Formula (J) is of Formula (I-C9). In some embodiments, a compound of Formula (J) is of Formula (I-C10). In some embodiments, a compound of Formula (J) is of Formula (I-C11). In some embodiments, a compound of Formula (J) is of Formula (I-C12). In some embodiments, a compound of Formula (J) is of Formula (I-C13). In some embodiments, a compound of Formula (J) is of Formula (I-C14). In some embodiments, a compound of Formula (J) is of Formula (I-C15). In some embodiments, a compound of Formula (J) is of Formula (I-C16). In some embodiments, a compound of Formula (J) is of Formula (I-C17). In some embodiments, a compound of Formula (J) is of Formula (I-C18). In some embodiments, a compound of Formula (J) is of Formula (I-C19). In some embodiments, a compound of Formula (J) is of Formula (I-C20). In some embodiments, a compound of Formula (J) is of Formula (I-C21). In some embodiments, a compound of Formula (J) is of Formula (I-C22). In some embodiments, a compound of Formula (J) is of Formula (I-C23).

In some embodiments of a compound of Formula (J), A, L, and B together with R⁵ and R⁶ form a moiety selected from the group consisting of:

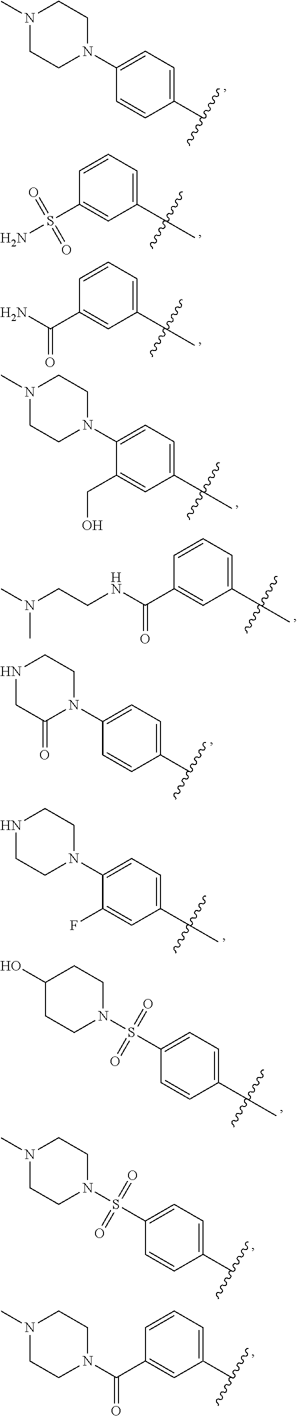

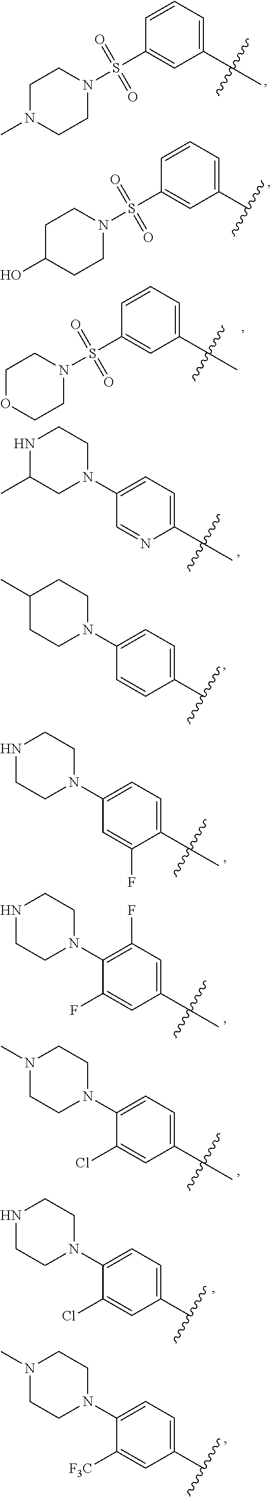

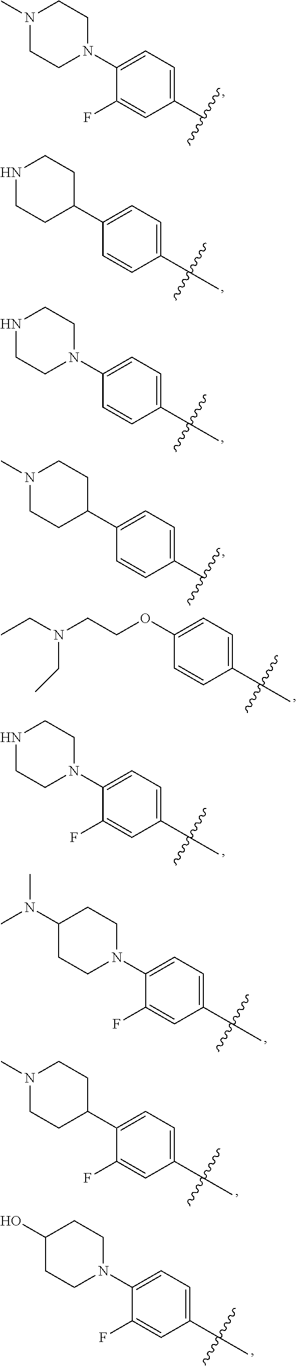

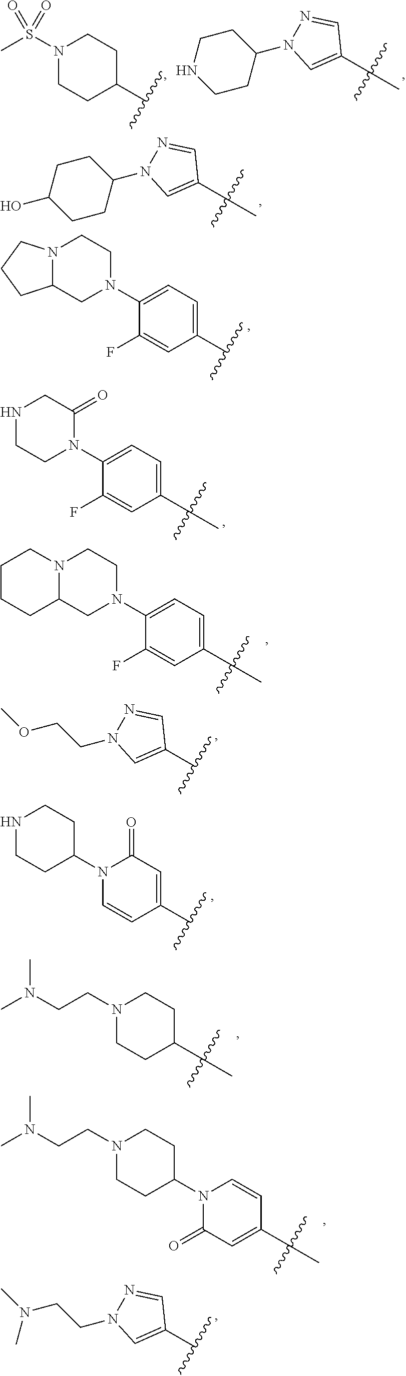

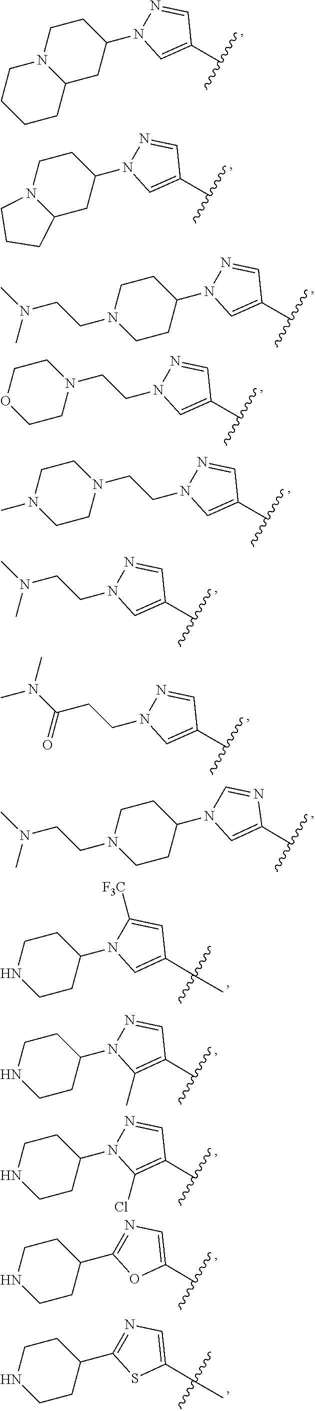

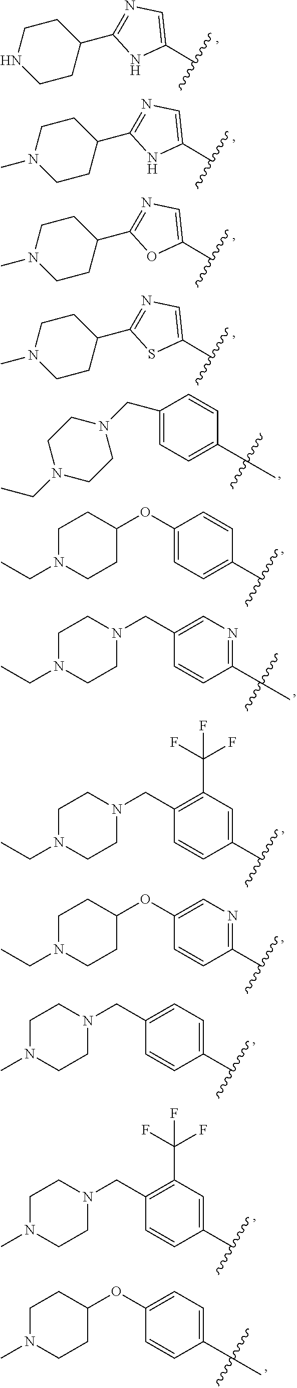

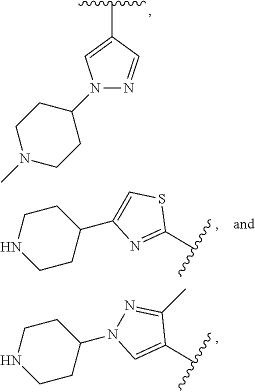

wherein the wavy lines denote attachment points to the parent molecule. In some embodiments of a compound of Formula (J), A, L, and B together with R⁵ and R⁶ form a moiety selected from the group consisting of:

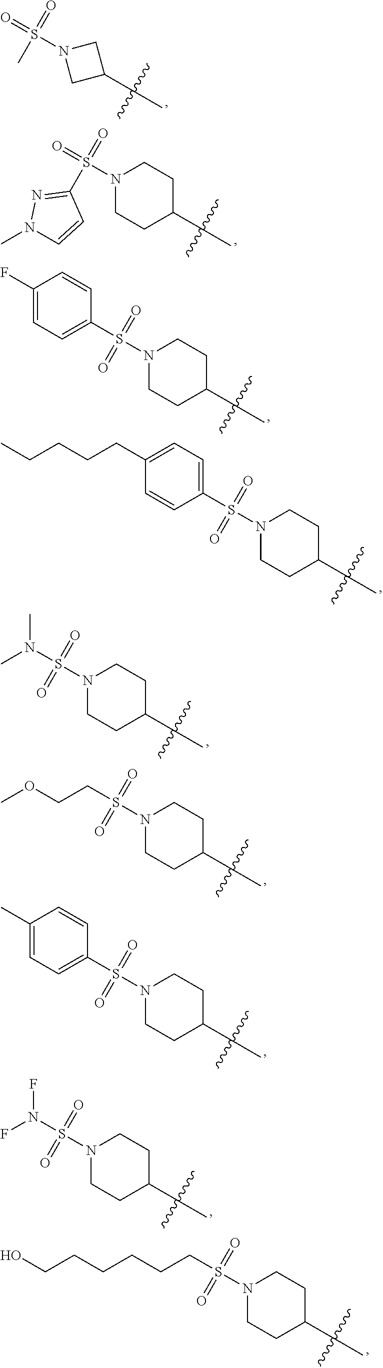

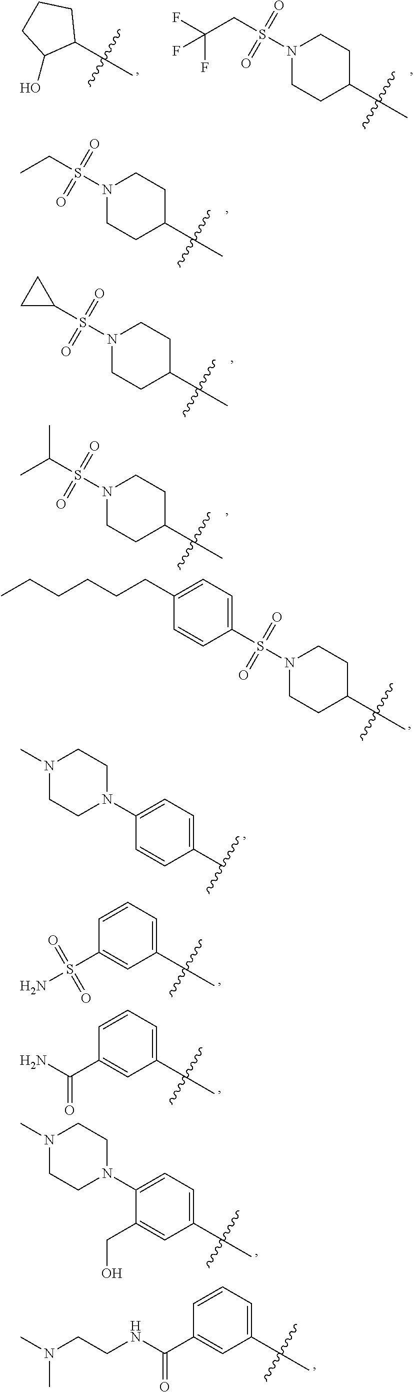

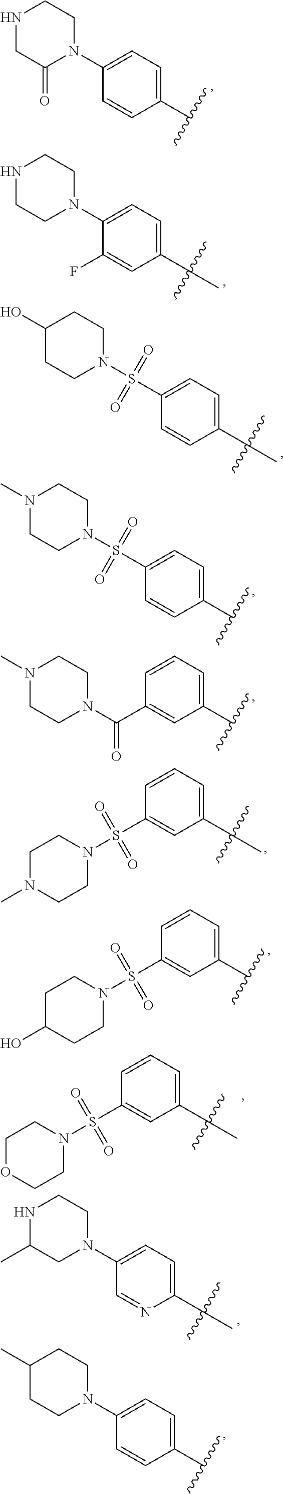

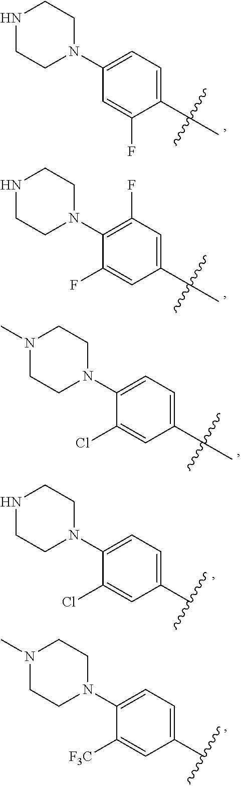

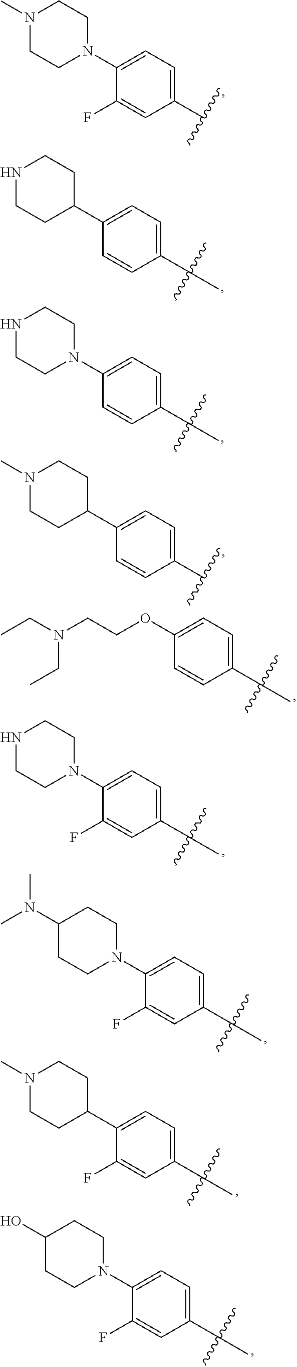

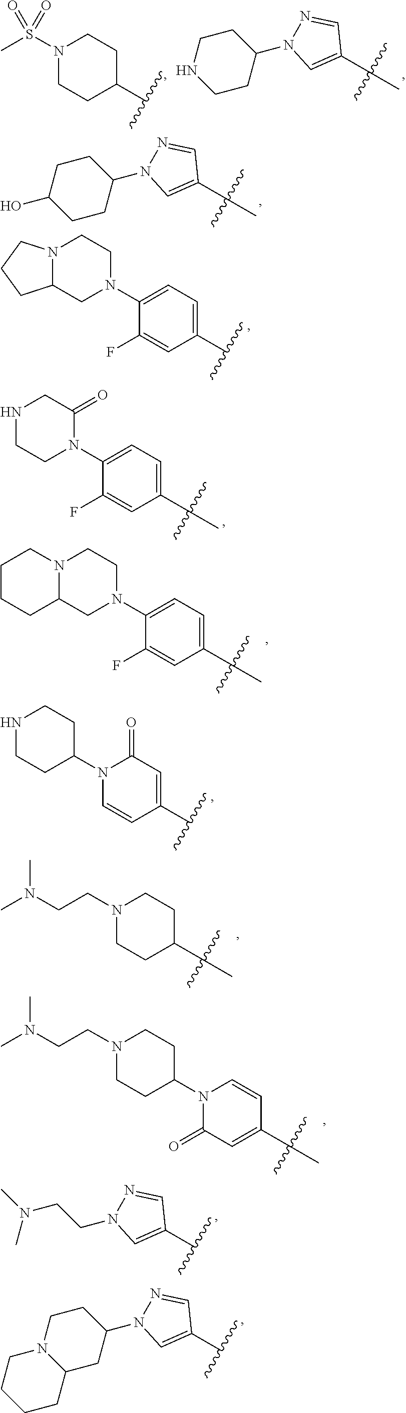

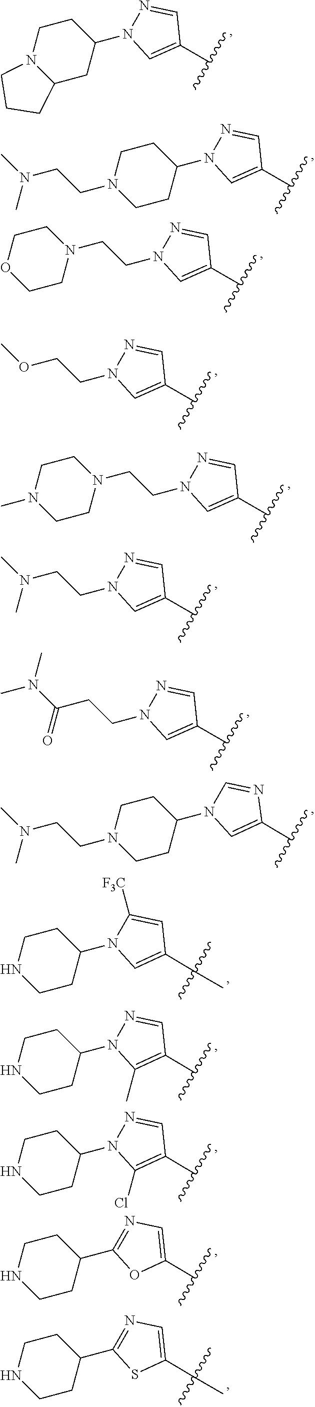

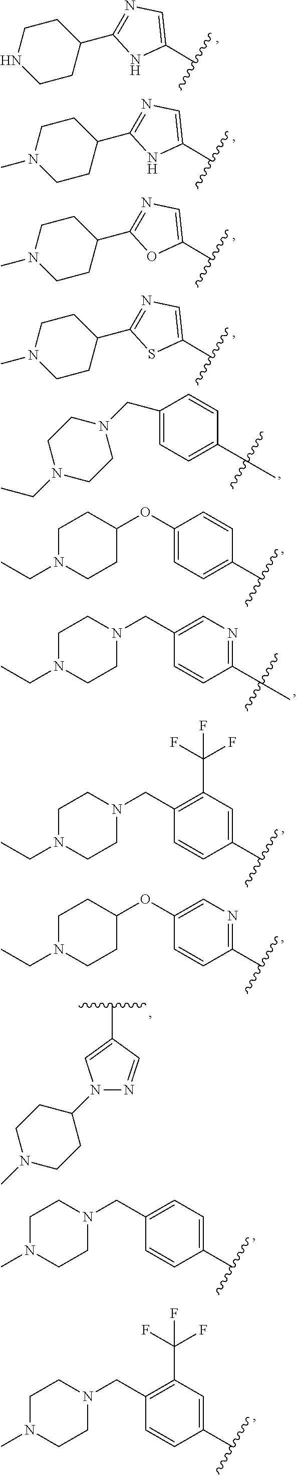

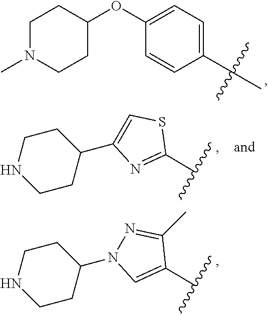

wherein the wavy lines denote attachment points to the parent molecule. It is understood that each description of A and B may be combined with each description of R¹, R², R³, and R⁴ the same as if each and every combination were specifically and individually listed. In some embodiments of a compound of Formula (K) or any related formulae where applicable, the moiety formed from A, L, and B together with R⁵ and R⁶ is as provided herein to the same extent as is described for Formula (J).

In some embodiments of a compound of Formula (J), W is

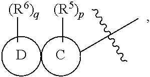

wherein D is fused with C to form a 7- to 12-membered bicyclic ring having at least one aromatic ring, wherein C and D are optionally substituted by R⁵ and R⁶. In some embodiments, W is

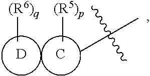

wherein D is fused with C to form a 7- to 12-membered bicyclic ring having one aromatic ring, wherein C and D are optionally substituted by R⁵ and R⁶. In some embodiments, W is

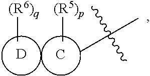

wherein D is fused with C to form a 7- to 12-membered bicyclic ring having at least one aromatic ring and at least one heteroatom selected from the group consisting of N, O, and S, wherein C and D are optionally substituted by R⁵ and R⁶. In some embodiments, W is

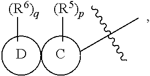

wherein D is fused with C to form a 7- to 12-membered bicyclic ring having one aromatic ring and at least one heteroatom selected from the group consisting of N, O, and S, wherein C and D are optionally substituted by R⁵ and R⁶. In some embodiments, W is

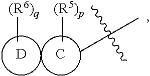

D is fused with C to form a 7- to 12-membered bicyclic ring having at least one aromatic ring and at least one nitrogen atom, wherein C and D are optionally substituted by R⁵ and R⁶. In some embodiments, W is

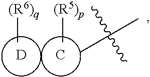

wherein D is fused with C to form a 7- to 12-membered bicyclic ring having one aromatic ring and at least one nitrogen atom, wherein C and D are optionally substituted by R⁵ and R⁶. In some embodiments of a compound of Formula (K) or any related formulae where applicable, W is as provided herein to the same extent as is described for Formula (J).

In some embodiments of a compound of Formula (J),

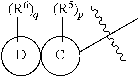

is selected from the group consisting of:

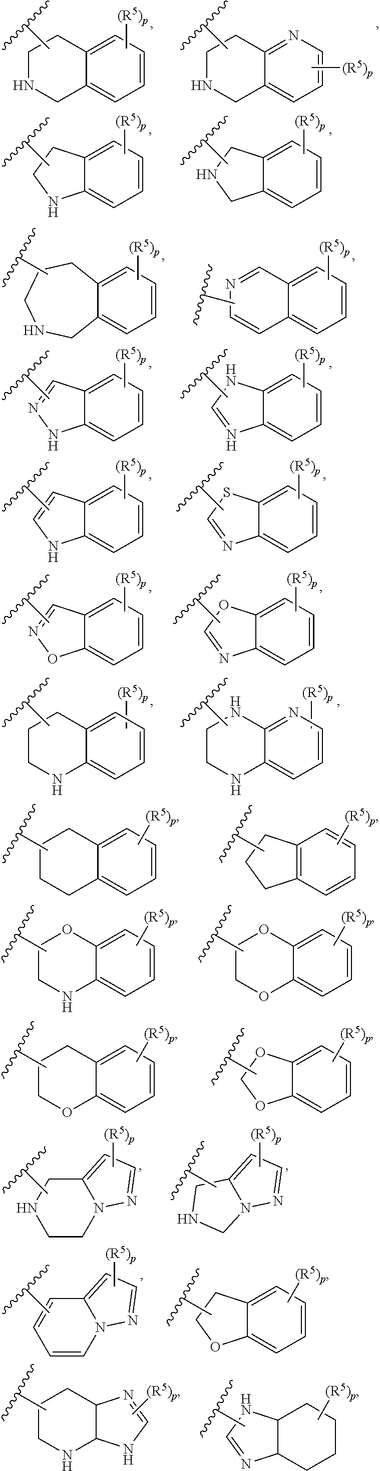

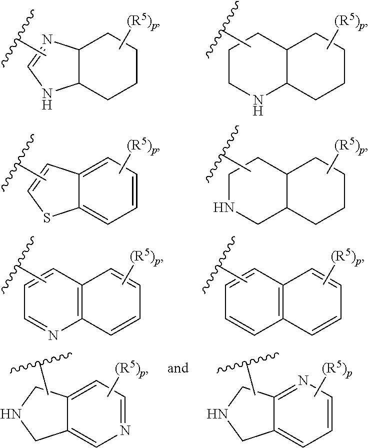

wherein the wavy lines denote attachment points to the parent molecule. In some embodiments of a compound of Formula (K) or any related formulae where applicable,

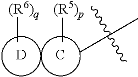

is as provided herein to the same extent as is described for Formula (J).

In some embodiments of a compound of Formula (J),

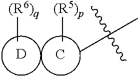

is selected from the group consisting of:

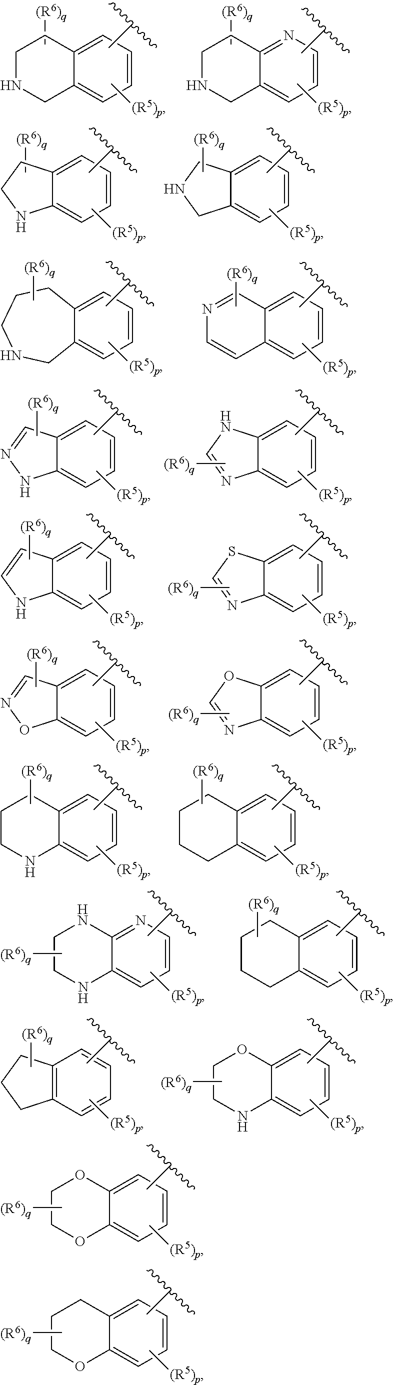

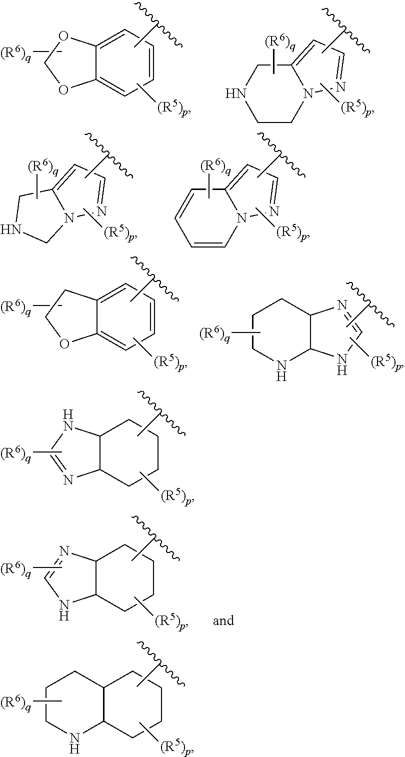

wherein the wavy lines denote attachment points to the parent molecule. In some embodiments of a compound of Formula (J),

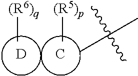

is selected from the group consisting of:

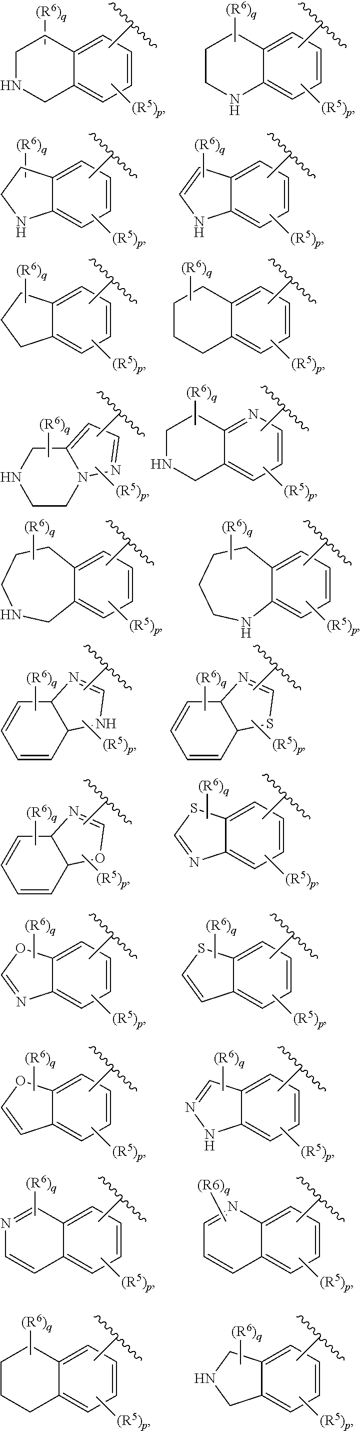

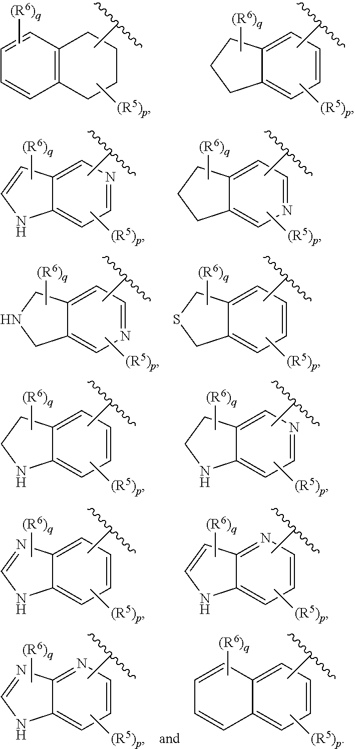

In some embodiments, W is

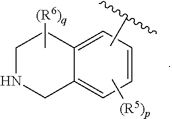

In some embodiments, W is

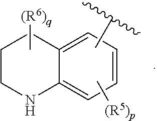

In some embodiments, W is

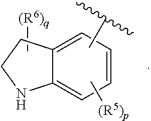

In some embodiments, W is

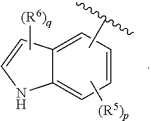

In some embodiments, W is

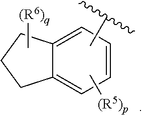

In some embodiments, W is

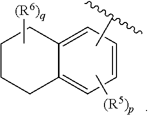

In some embodiments, W is

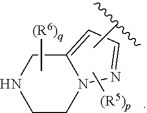

In some embodiments, W is

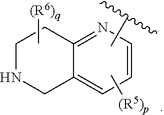

In some embodiments, W is

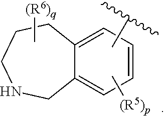

In some embodiments, W is

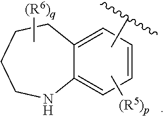

In some embodiments, W is

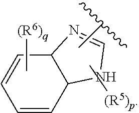

In some embodiments, W is

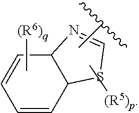

In some embodiments, W is

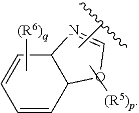

In some embodiments, W is

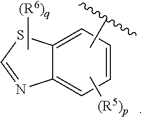

In some embodiments, W is

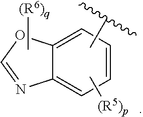

In some embodiments, W is

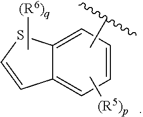

In some embodiments, W is

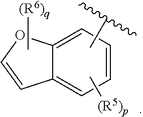

In some embodiments, W is

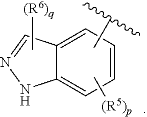

In some embodiments, W is

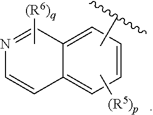

In some embodiments, W is

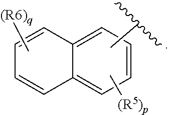

In some embodiments, W is

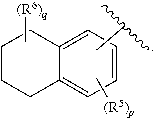

In some embodiments, W is

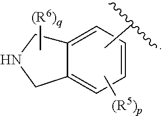

In some embodiments, W is

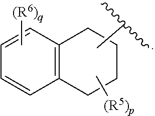

In some embodiments, W is

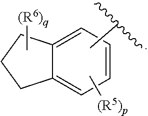

In some embodiments, W is

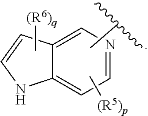

In some embodiments, W is

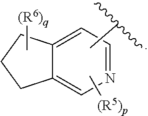

In some embodiments, W is

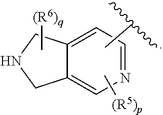

In some embodiments, W is

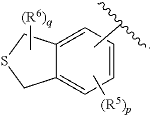

In some embodiments, W is

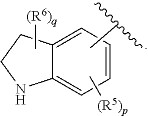

In some embodiments, W is

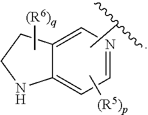

In some embodiments, W is

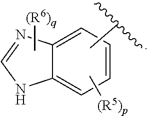

In some embodiments, W is

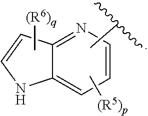

In some embodiments, W is

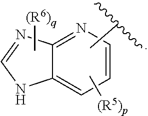

In some embodiments, W is

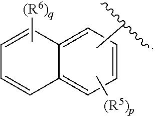

In some embodiments of a compound of Formula (K) or any related formulae where applicable,

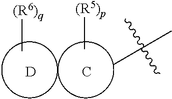

is as provided herein to the same extent as is described for Formula (J).

In some embodiments of a compound of Formula (J), C-D, R⁵ and R⁶ together are selected from the group consisting of:

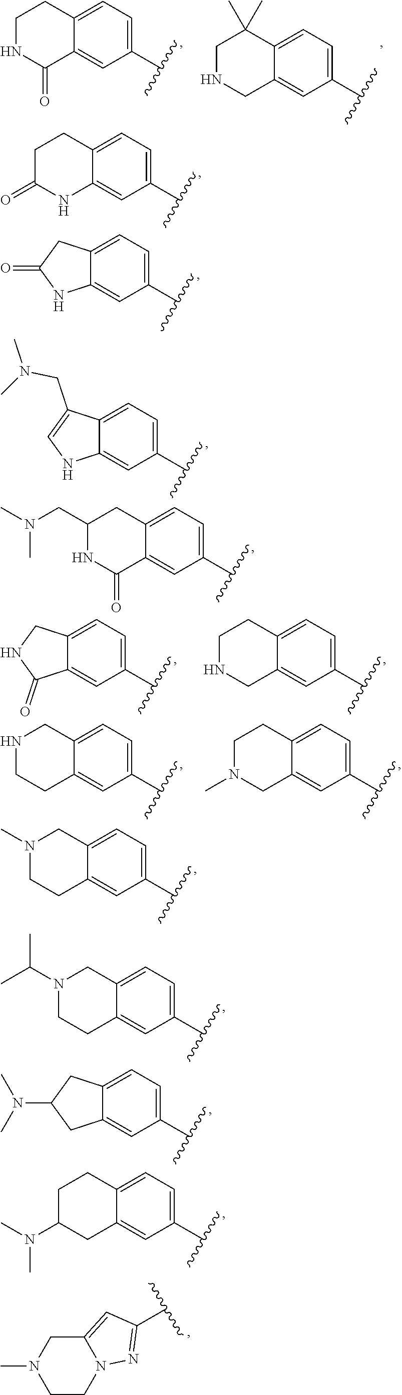

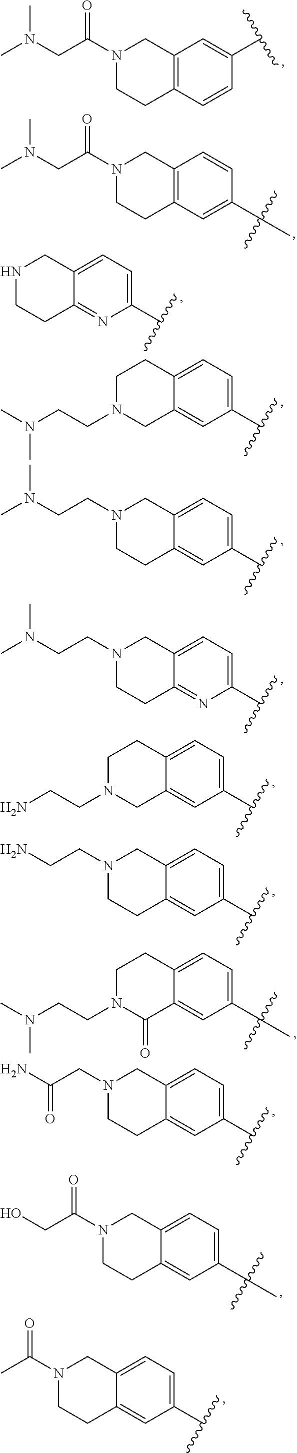

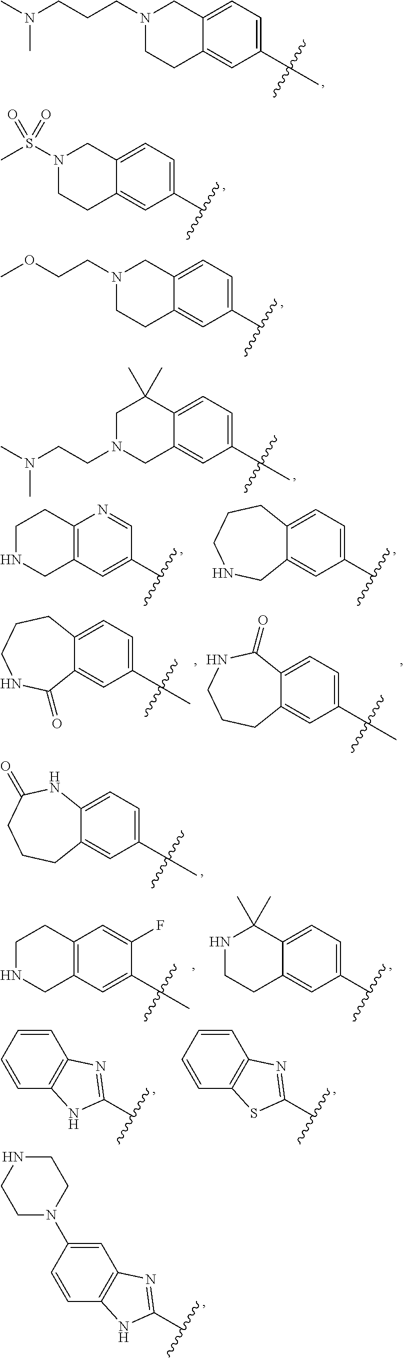

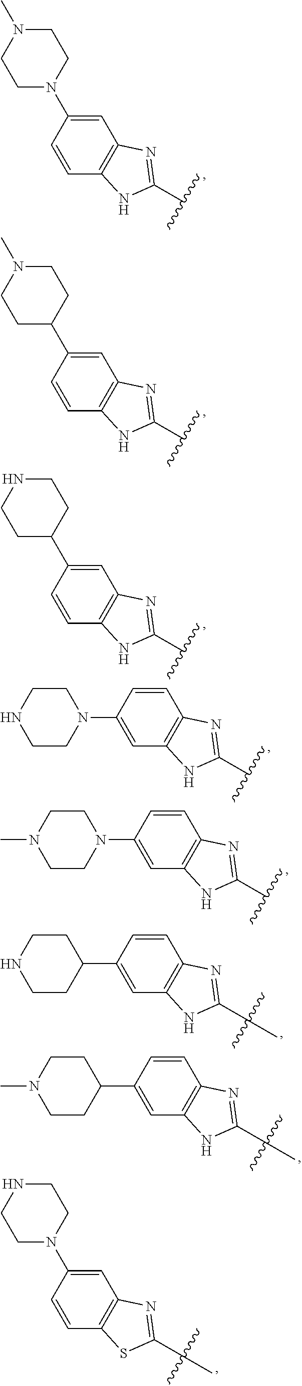

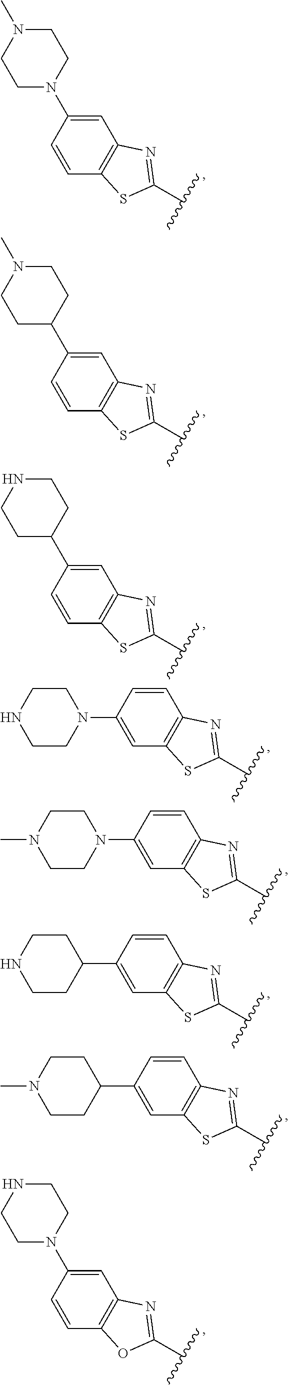

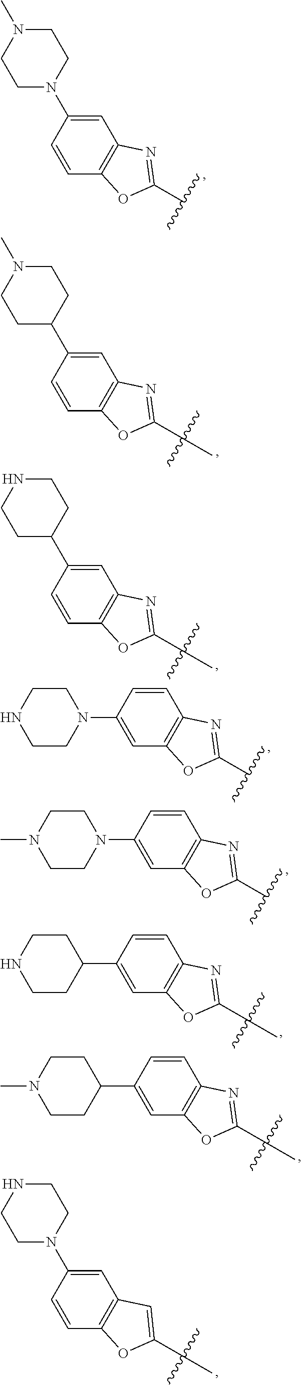

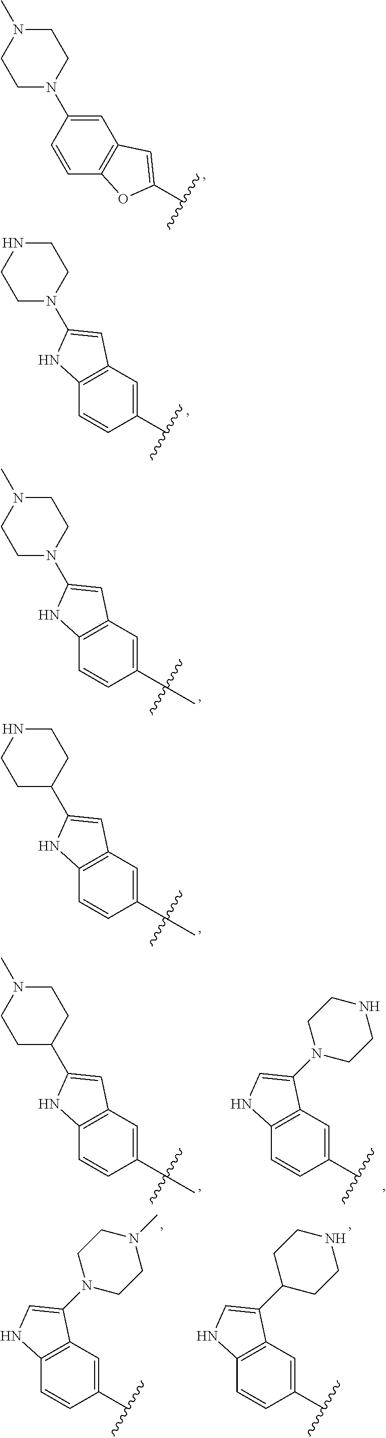

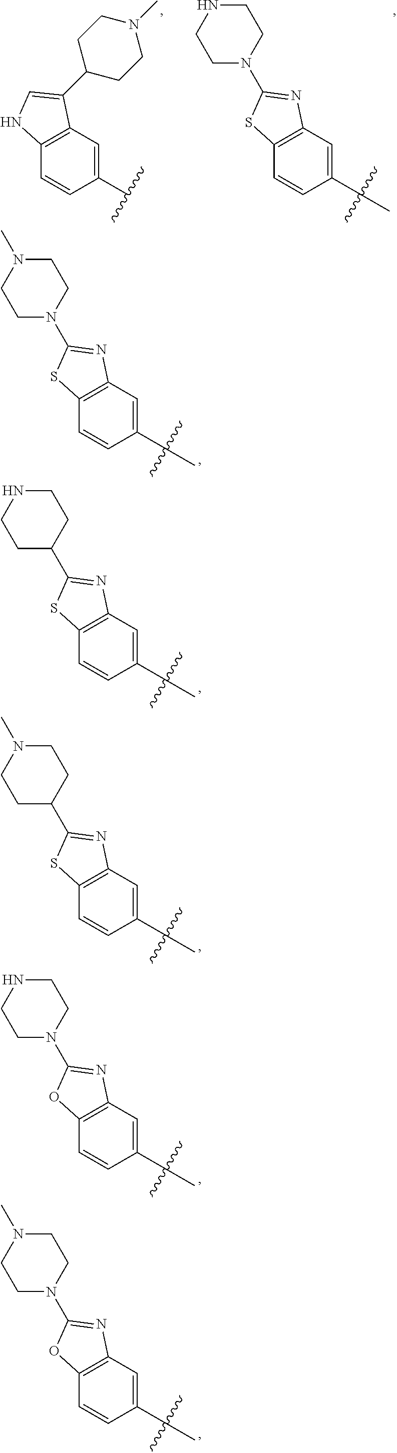

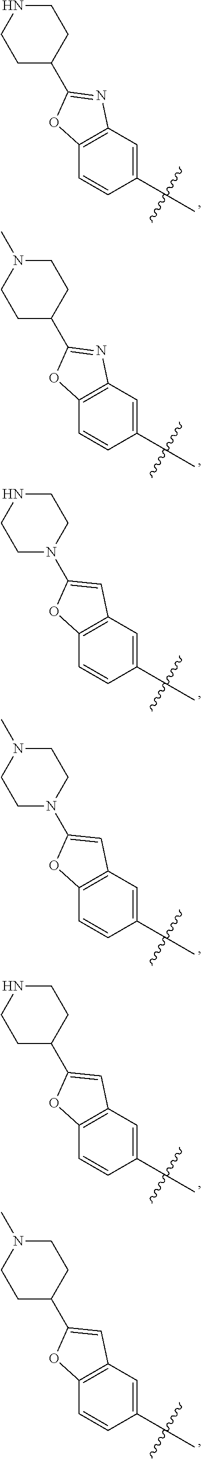 [SMILES: *c1ccc2oc(C3CCNCC3)nc2c1 ; *c1ccc2oc(C3CCN(C)CC3)nc2c1 ; *c1ccc2oc(N3CCNCC3)cc2c1 ; *c1ccc2oc(N3CCN(C)CC3)cc2c1 ; *c1ccc2oc(C3CCNCC3)cc2c1 ; *c1ccc2oc(C3CCN(C)CC3)cc2c1]

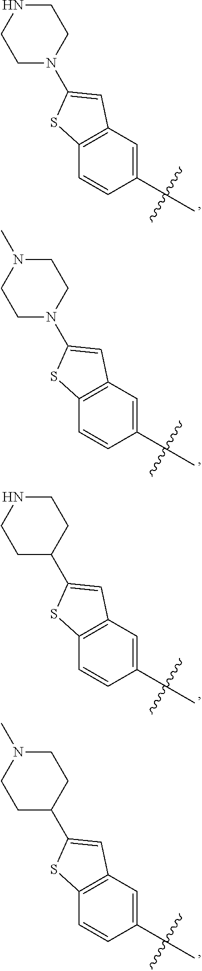 [SMILES: *c1ccc2sc(N3CCNCC3)cc2c1 ; *c1ccc2sc(N3CCN(C)CC3)cc2c1 ; *c1ccc2sc(C3CCNCC3)cc2c1 ; *c1ccc2sc(C3CCN(C)CC3)cc2c1]

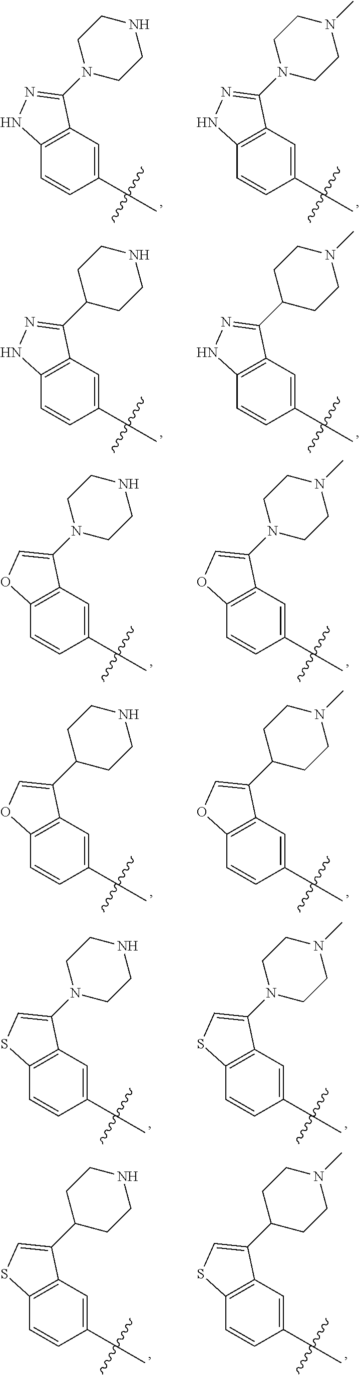

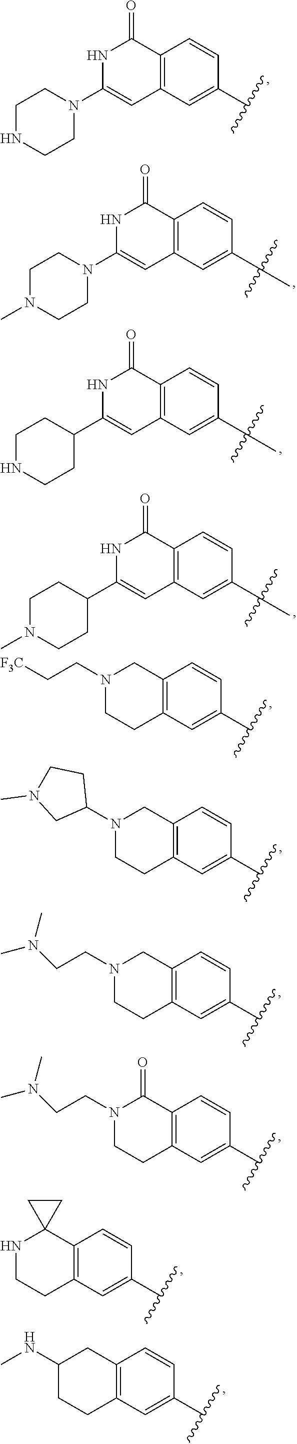

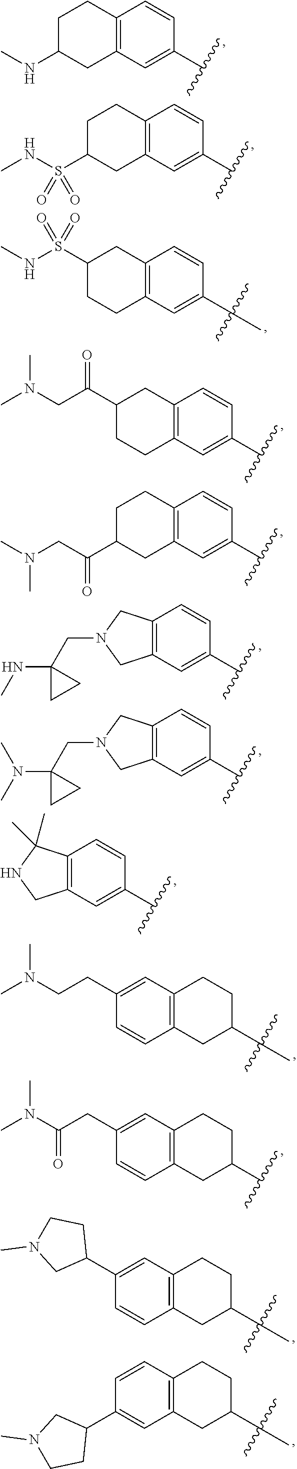

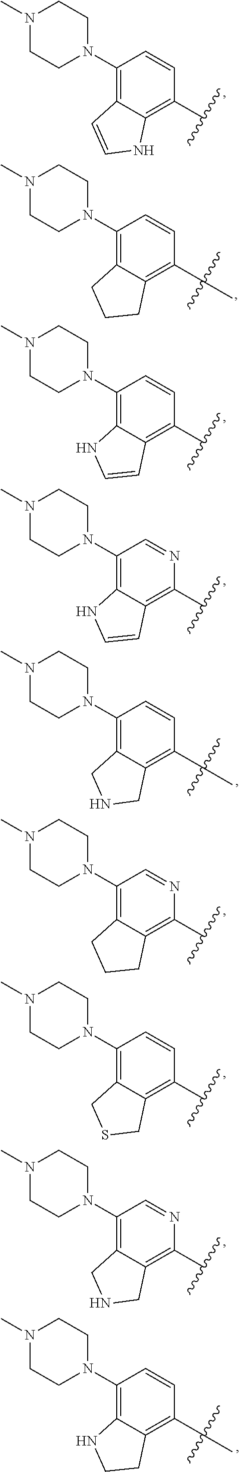

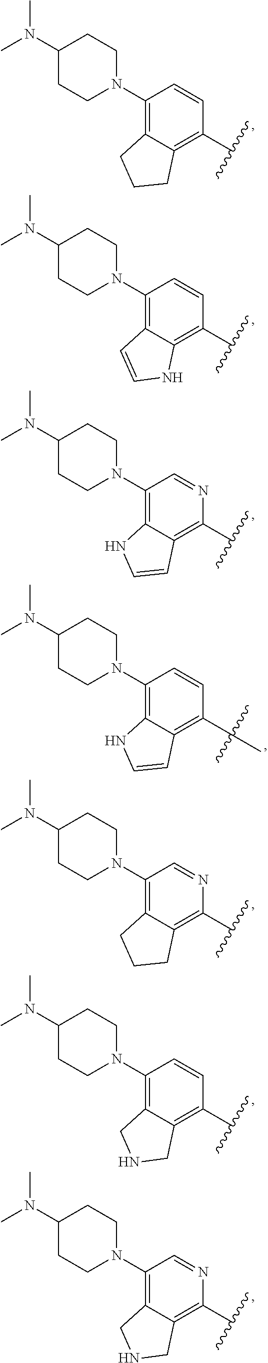

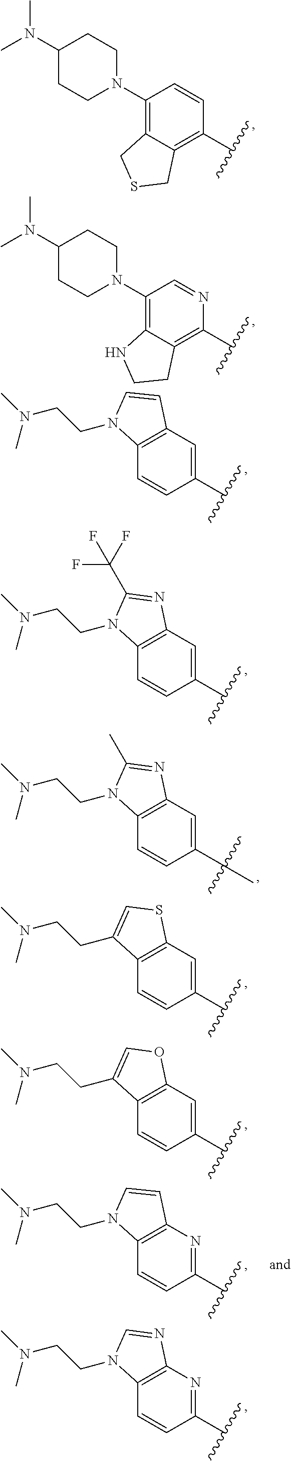

wherein the wavy lines denote attachment points to the parent molecule. In some embodiments of a compound of Formula (K) or any related formulae where applicable, C-D, R⁵ and R⁶ together are as provided herein to the same extent as is described for Formula (J).

In some embodiments of a compound of Formula (J), p is 0. In some embodiments, p is 0 or 1. In some embodiments, p is 0, 1, or 2. In some embodiments, p is 0, 1, 2, or 3. In some embodiments, p is 1. In some embodiments, p is 2. In some embodiments, p is 3. In some embodiments, p is 4. In some embodiments, p is 1, 2, 3, or 4. In some embodiments, p is 1, 2, or 3. In some embodiments, p is 1 or 2. In some embodiments, p is 2 or 3. In some embodiments of a compound of Formula (K) or any related formulae where applicable, p is as provided herein to the same extent as is described for Formula (J).

In some embodiments of a compound of Formula (J), each R⁵ is independently —S(O)₂R¹⁰, —S(O)₂NR¹¹R¹², —C(O)NR¹¹R¹², —(C₁-C₃ alkylene)OR¹⁰, 3- to 12-membered heterocyclyl, —(C₁-C₃ alkylene)NR¹¹R¹², halogen, C₁-C₆ alkyl, —OR¹⁰, or oxo, each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen. In some embodiments, each R⁵ is independently —S(O)₂NH₂, —C(O)NH₂, —CH₂OH, —C(O)NH(CH₂)₂N(CH₃)₂, fluoro, chloro, oxo, —CF₃, —O(CH₂)₂N(CH₂CH₃)₂, piperazinyl optionally substituted by methyl or —N(CH₃)₂, or piperidiny optionally substituted by methyl or —N(CH₃)₂. In some embodiments of a compound of Formula (K) or any related formulae where applicable, R⁵ is as provided herein to the same extent as is described for Formula (J).

In some embodiments of a compound of Formula (J), q is 0. In some embodiments, q is 0 or 1. In some embodiments, q is 0, 1, or 2. In some embodiments, q is 0, 1, 2, or 3. In some embodiments, q is 1. In some embodiments, q is 2. In some embodiments, q is 3. In some embodiments, q is 4. In some embodiments, q is 1, 2, 3, or 4. In some embodiments, q is 1, 2, or 3. In some embodiments, q is 1 or 2. In some embodiments, q is 2 or 3. In some embodiments of a compound of Formula (K) or any related formulae where applicable, q is as provided herein to the same extent as is described for Formula (J).

In some embodiments of a compound of Formula (J), each R⁶ is independently C₁-C₆ alkyl, oxo, —OR¹⁰, —(C₁-C₃ alkylene)NR¹¹R¹², C₃-C₆ cycloalkyl, 3- to 12-membered heterocyclyl, —S(O)₂NR¹¹R¹², —NR¹¹R¹², —C(O)R¹⁰, —(C₁-C₃ alkylene)C(O)NR¹¹R¹², —S(O)₂R¹⁰, —(C₁-C₃ alkylene)(C₃-C₆ cycloalkyl), —(C₁-C₃ alkylene)(3- to 12-membered heterocyclyl), or —(C₁-C₃ alkylene)OR¹⁰, each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen; or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each independently optionally substituted by C₁-C₆ alkyl. In some embodiments, each R⁶ is independently methyl, ethyl, oxo, —OH, —CH₂N(CH₃)₂, isopropyl, —N(CH₃)₂, —C(O)CH₂N(CH₃)₂, —CH₂C(O)N(CH₃)₂, —(CH₂)₂N(CH₃)₂, —(CH₂)₂NH₂, —CH₂C(O)NH₂, —C(O)CH₂OH, —C(O)CH₃, —(CH₂)₃N(CH₃)₂, —S(O)₂CH₃, —(CH₂)₂OCH₃, piperazinyl optionally substituted by methyl, —(CH₂)₂CF₃, pyrrolidinyl optionally substituted by methyl, —NHCH₃, —S(O)₂NHCH₃, azetidinyl optionally substituted by methyl,

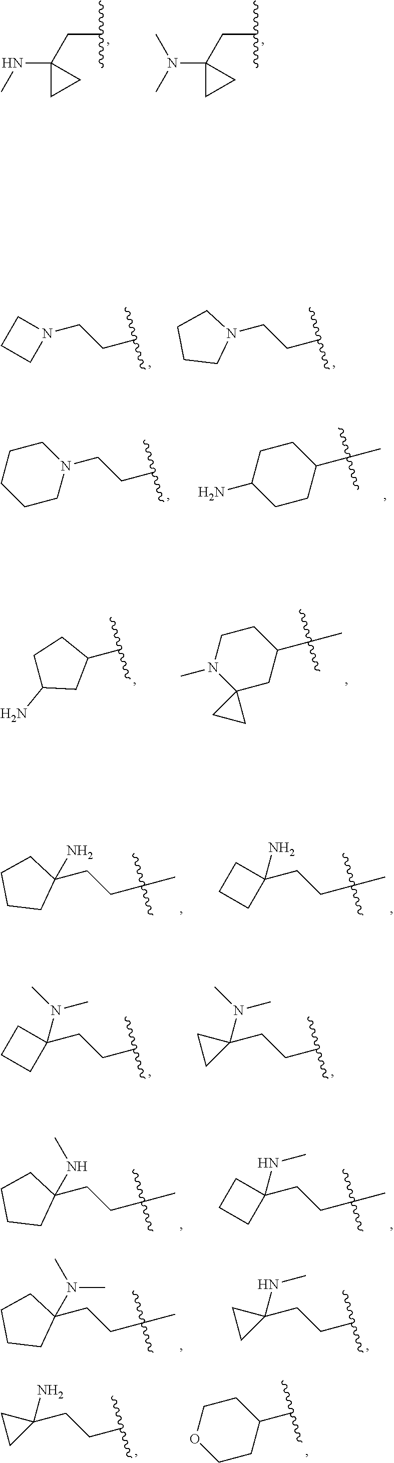

or piperidiny optionally substituted by methyl, ethyl, isopropyl, —C(O)CH₃, —C(O)OCH₃, —CH₂CHF₂, —(CH₂)₂OH, —(CH₂)₂S(O)₂CH₃, —(CH₂)₂OCH₃, or —CH₂CF₃. In some embodiments of a compound of Formula (K) or any related formulae where applicable, R⁶ is as provided herein to the same extent as is described for Formula (J).

In some embodiments of a compound of Formula (J), X is CR^{a}. In some embodiments, X is CR^{a} and R^{a} is hydrogen. In some embodiments, X is N. In some embodiments of a compound of Formula (K) or any related formulae where applicable, X is as provided herein to the same extent as is described for Formula (J).

In some embodiments of a compound of Formula (J), Y is CR^{b}. In some embodiments, Y is CR^{b} and R^{b} is hydrogen, —CN, or —NR¹¹R¹². In some embodiments, Y is CR^{b} and R^{b} is hydrogen, —CN, or —NHCH₃. In some embodiments, Y is N. In some embodiments of a compound of Formula (K) or any related formulae where applicable, Y is as provided herein to the same extent as is described for Formula (J).

In some embodiments a compound of Formula (J), X is CR^{a} and Y is N. In some embodiments, X is CH and Y is N. In some embodiments, X is N and Y is CH. In some embodiments, X is N and Y is CR^{b}.In some embodiments, X is N and Y is N. In some embodiments of a compound of Formula (K) or any related formulae where applicable, X and Y are as provided herein to the same extent as is described for Formula (J).

In some embodiments of a compound of Formula (J), Q is O. In some embodiments, Q is S. In some embodiments, Q is O; X is N; and Y is N. In some embodiments of a compound of Formula (K) or any related formulae where applicable, Q is as provided herein to the same extent as is described for Formula (J).

In some embodiments of a compound of Formula (J), R is —CN. In some embodiments, R is C₁-C₆ haloalkyl. In some embodiments, R is C₁-C₆ haloalkyl, wherein the halogen in the C₁-C₆ haloalkyl is fluoro. In some embodiments, R is —CH₂F, —CHF₂, —CF₃, or —CF₂CH₃. In some embodiments, R is —CN, —CH₂F, —CHF₂, —CF₃, or —CF₂CH₃. In some embodiments, R is —CH₂F. In some embodiments, R is —CHF₂. In some embodiments, R is —CF₃. In some embodiments, R is —CF₂CH₃.

In some embodiments of a compound of Formula (J), R is C₃-C₆ cycloalkyl. In some embodiments, R is cyclopropyl. In some embodiments, R is —CN, C₁-C₆ haloalkyl, or C₁-C₆ cycloalkyl. In some embodiments, R is —CN, —CH₂F, —CHF₂, —CF₃, —CF₂CH₃, or cyclopropyl. In some embodiments of a compound of Formula (K) or any related formulae where applicable, R is as provided herein to the same extent as is described for Formula (J).

In some embodiments of a compound of Formula (J), Formula (I), or Formula (II), R¹ is C₁-C₆ alkyl, C₃-C₆ cycloalkyl, 3- to 12-membered heterocyclyl, —(C₁-C₃ alkylene)(C₆-C₁₄ aryl), C₆-C₁₄ aryl, or —(C₁-C₃ alkylene)(C₃-C₆ cycloalkyl), each of which is independently optionally substituted by halogen, —OR¹³, or C₁-C₆ alkyl optionally substituted by oxo, —OH, or halogen. In some embodiments, R¹ is C₁-C₆ alkyl optionally substituted by —OH or halogen. In some embodiments, R¹ is C₃-C₆ cycloalkyl optionally substituted by halogen, —OR¹³, or C₁-C₆ alkyl optionally substituted by oxo, —OH, or halogen. In some embodiments, R¹ is selected from the group consisting of:

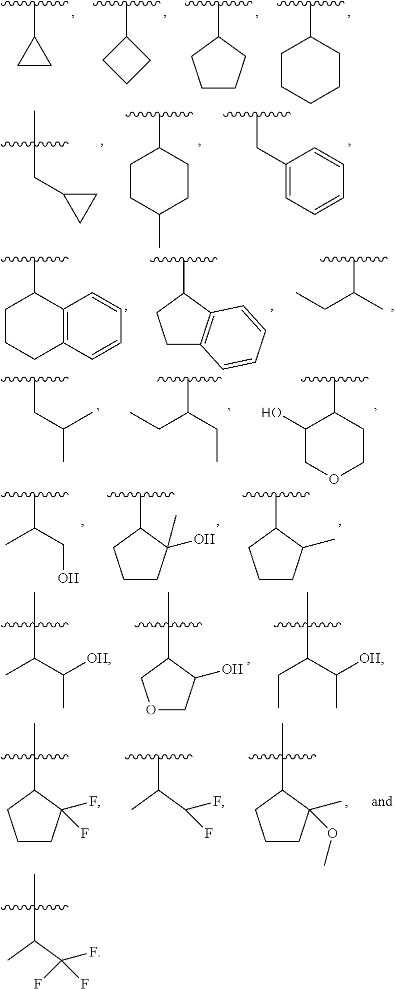

In some embodiments, R¹ is

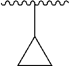 [SMILES: *C1CC1]

In some embodiments, R¹ is

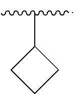 [SMILES: *C1CCC1]

In some embodiments, R¹ is

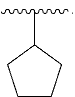 [SMILES: *C1CCCC1]

In some embodiments, R¹ is

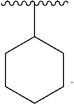 [SMILES: *C1CCCCC1]

In some embodiments, R¹ is

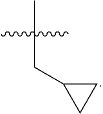 [SMILES: *CC1CC1]

In some embodiments, R¹ is

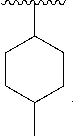 [SMILES: *C1CCC(C)CC1]

In some embodiments, R¹ is

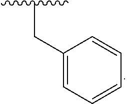 [SMILES: *Cc1ccccc1]

In some embodiments, R¹ is

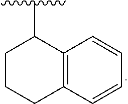 [SMILES: *C1CCCc2ccccc21]

In some embodiments, R¹ is

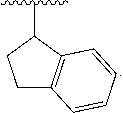 [SMILES: *C1CCc2ccccc21]

In some embodiments, R¹ is

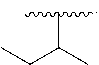 [SMILES: *C(C)CC]

In some embodiments, R¹ is

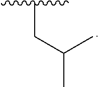 [SMILES: *CC(C)C]

In some embodiments, R¹ is

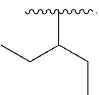 [SMILES: *C(CC)CC]

In some embodiments, R¹ is

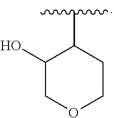 [SMILES: *C1CCOCC1O]

In some embodiments, R¹ is

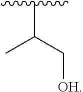 [SMILES: *C(C)CO]

In some embodiments, R¹ is

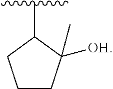 [SMILES: *C1CCCC1(C)O]

In some embodiments, R¹ is

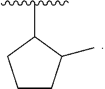 [SMILES: *C1CCCC1C]

In some embodiments, R¹ is

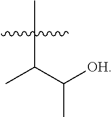 [SMILES: *C(C)C(C)O]

In some embodiments, R¹ is

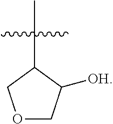 [SMILES: *C1COCC1O]

In some embodiments, R¹ is

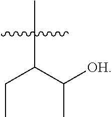 [SMILES: *C(CC)C(C)O]

In some embodiments, R¹ is

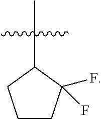 [SMILES: *C1CCCC1(F)F]

In some embodiments, R¹ is

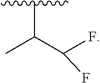 [SMILES: *C(C)C(F)F]

In some embodiments, R¹ is

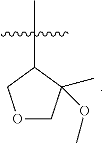 [SMILES: *C1COCC1(C)OC]

In some embodiments, R¹ is

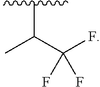 [SMILES: *C(C)C(F)(F)F]

In some embodiments of a compound of Formula (K) or any related formulae where applicable, R¹ is as provided herein to the same extent as is described for Formula (J).

In some embodiments of a compound of Formula (K), R¹ is C₃-C₈ cycloalkyl. In some embodiments, R¹ is

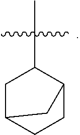 [SMILES: *C1CC2CCC1C2]

In some embodiments, R¹ is

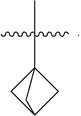 [SMILES: *C12CC(C1)C2]

In some embodiments, R¹ is

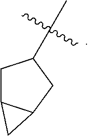 [SMILES: *C1CC2CC2C1]

In some embodiments, R¹ is

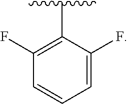 [SMILES: *c1c(F)cccc1F]

In some embodiments, R¹ is

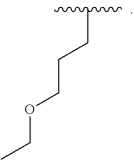 [SMILES: *CCCOCC]

In some embodiments, R¹ is selected from the group consisting of:

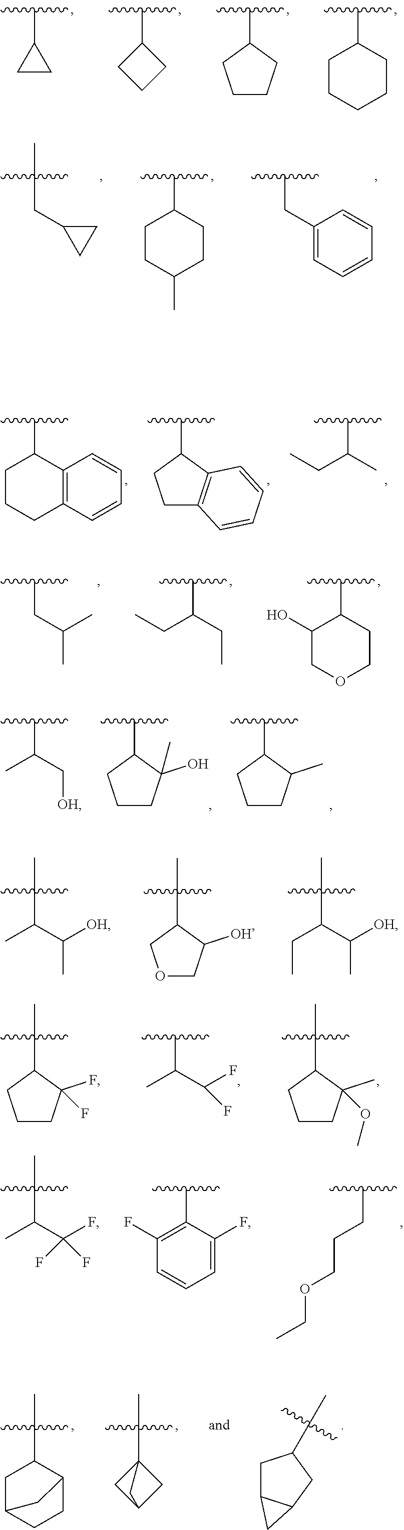

In some embodiments of a compound of Formula (J), R² is hydrogen. In some embodiments, R² is C₁-C₆ alkyl. In some embodiments, R² is C₃-C₆ cycloalkyl. In some embodiments, R² is C₁-C₆ haloalkyl. In some embodiments, R² is C₁-C₆ alkoxy. In some embodiments, R² is C₁-C₆ haloalkoxy. In some embodiments, R² is halogen. In some embodiments, R² is —CN. In some embodiments, R² is —C(O)R¹⁰. In some embodiments, R² is —C(O)NR¹¹R¹². In some embodiments of a compound of Formula (K) or any related formulae where applicable, R² is as provided herein to the same extent as is described for Formula (J).

In some embodiments of a compound of Formula (J), R³ is hydrogen. In some embodiments, R³ is C₁-C₆ alkyl. In some embodiments, R³ is C₃-C₆ cycloalkyl. In some embodiments, R³ is C₁-C₆ haloalkyl. In some embodiments, R³ is C₁-C₆ alkoxy. In some embodiments, R³ is C₁-C₆ haloalkoxy. In some embodiments, R³ is halogen. In some embodiments, R³ is —CN. In some embodiments, R³ is —C(O)R¹⁰. In some embodiments, R³ is —C(O)NR¹¹R¹². In some embodiments of a compound of Formula (K) or any related formulae where applicable, R³ is as provided herein to the same extent as is described for Formula (J).

In some embodiments of a compound of Formula (J), R² is hydrogen and R³ is hydrogen. In some embodiments of a compound of Formula (K) or any related formulae where applicable, R² and R³ are as provided herein to the same extent as is described for Formula (J).

In some embodiments of a compound of Formula (J), R⁴ is hydrogen. In some embodiments, R⁴ is C₁-C₆ alkyl. In some embodiments, R² is hydrogen; R³ is hydrogen; and R⁴ is hydrogen. In some embodiments of a compound of Formula (K) or any related formulae where applicable, R⁴ is as provided herein to the same extent as is described for Formula (J).

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ for a compound of Formula (J) as well as all related formulae such as Formula (I), (I-B1) to (I-B22), (I-C1) to (I-C23), and Formula (II) may be combined with every description, variation, embodiment or aspect for one or more of the variables X, Y, R, Q, A, B, C, D, R², R³, R⁴, R⁵, R⁶, L, p, and q the same as if each and every combination were specifically and individually listed. It is to be understood that any variable for a compound of Formula (K) or any related formulae may be combined with any other variable the same as if each and every combination of variables were specifically and individually listed. For example, in one aspect, provided is a compound of Formula (J) or a salt thereof, wherein R is —CN; Q is O; X is N; Y is N; R¹ is C₁-C₆ alkyl, C₃-C₆ cycloalkyl, 3- to 12-membered heterocyclyl, —(C₁-C₃ alkylene)(C₆-C₁₄ aryl), C₆-C₁₄ aryl, or —(C₁-C₃ alkylene)(C₃-C₆ cycloalkyl), each of which is independently optionally substituted by halogen, —OR¹³, or C₁-C₆ alkyl optionally substituted by oxo, —OH, or halogen; R² is hydrogen; R³ is hydrogen; R⁴ is hydrogen; W is

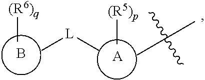

such as a moiety that provides a compound of any one of Formula (I-B1) to (I-B22) and (I-C1) to (I-C23). As another example, in another aspect, provided is a compound of Formula (J) or a salt thereof, wherein R is —CN; Q is O; X is N; Y is N; R¹ is C₁-C₆ alkyl, C₃-C₆ cycloalkyl, 3- to 12-membered heterocyclyl, —(C₁-C₃ alkylene)(C₆-C₁₄ aryl), C₆-C₁₄ aryl, or —(C₁-C₃ alkylene)(C₃-C₆ cycloalkyl), each of which is independently optionally substituted by halogen, —OR¹³, or C₁-C₆ alkyl optionally substituted by oxo, —OH, or halogen; R² is hydrogen; R³ is hydrogen; R⁴ is hydrogen; W is

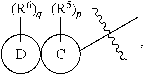

such as a moiety selected from the group consisting of

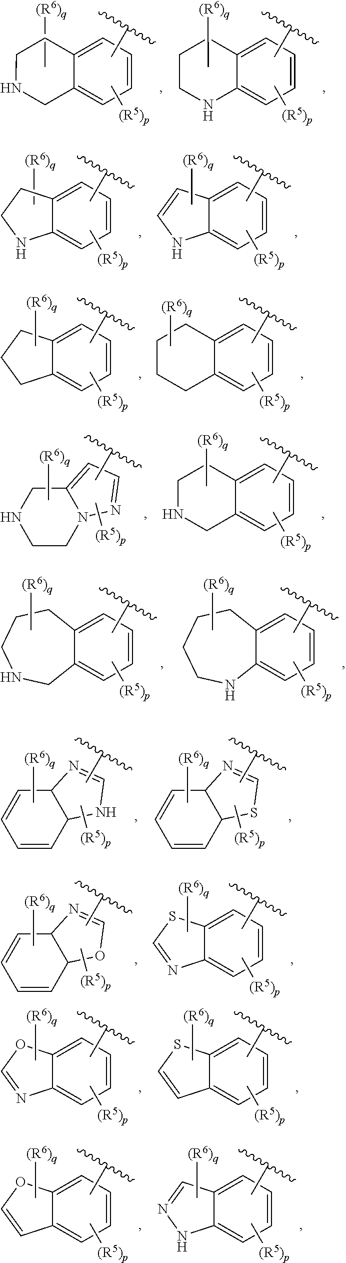

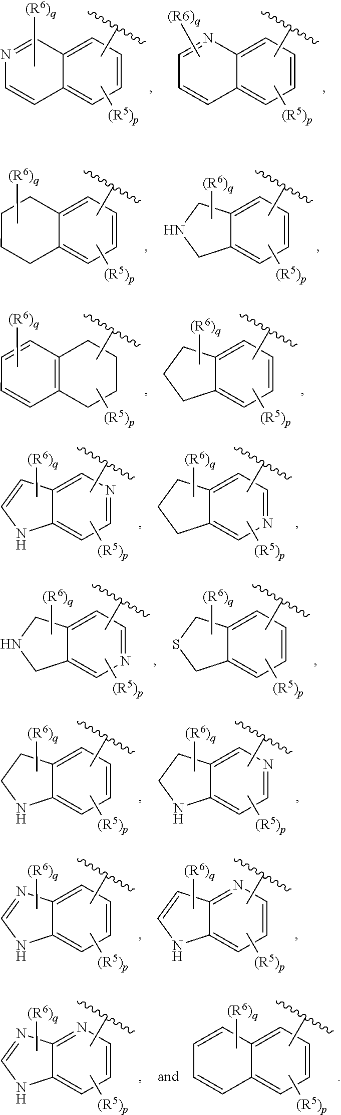

Any embodiment provided for Formula (J) is equally applicable to other formulae where applicable, such as Formula (K), the same as if each and every embodiment were specifically and individually listed.

In some embodiments of a compound of Formula (K) or any related formulae where applicable, when R is C₁-C₆ haloalkyl and R¹ is C₃-C₁₂ cycloalkyl, then B is hydrogen, C₃-C₆ cycloalkyl, 3- to 10-membered heterocyclyl, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶.

Also provided are salts of compounds referred to herein, such as pharmaceutically acceptable salts. The invention also includes any or all of the stereochemical forms, including any enantiomeric or diastereomeric forms, and any tautomers or other forms of the compounds described. It is understood that individual enantiomers and diastereomers are provided herein and their corresponding structures can be readily determined.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds are listed in Table 1.

TABLE 1

| Compound | |
|---|---|
| no. | Structure |
| 1 | 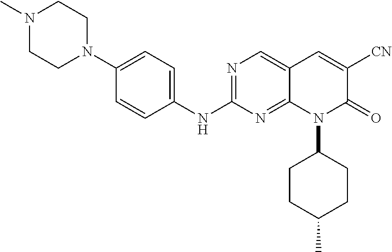 |
| 2 | 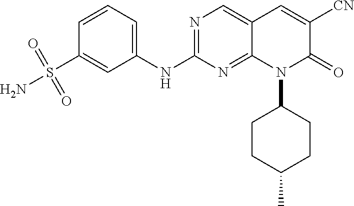 |
| 3 | 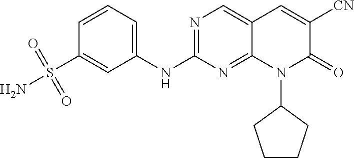 [SMILES: N#Cc1cc2cnc(Nc3cccc(S(N)(=O)=O)c3)nc2n(C2CCCC2)c1=O] |
| 4 | 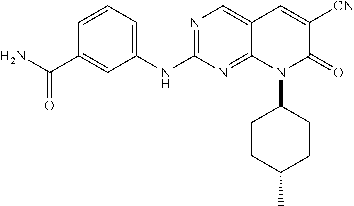 |
| 5 | 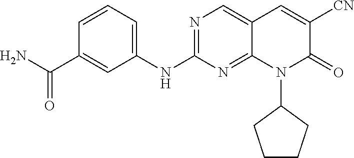 [SMILES: N#Cc1cc2cnc(Nc3cccc(C(N)=O)c3)nc2n(C2CCCC2)c1=O] |
| 6 | 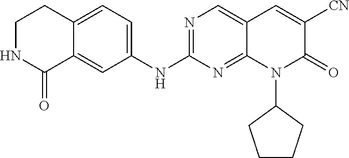 [SMILES: N#Cc1cc2cnc(Nc3ccc4c(c3)C(=O)NCC4)nc2n(C2CCCC2)c1=O] |
| 7 | 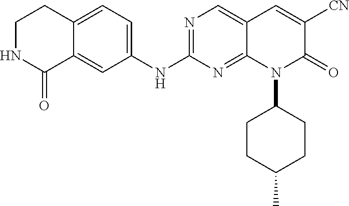 |
| 8 | 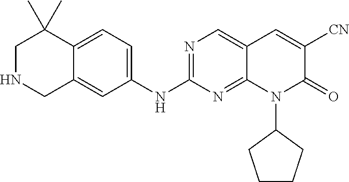 [SMILES: CC1(C)CNCc2cc(Nc3ncc4cc(C#N)c(=O)n(C5CCCC5)c4n3)ccc21] |
| 9 | 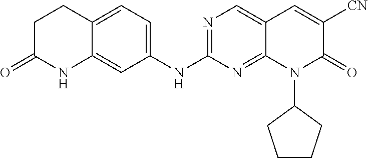 [SMILES: N#Cc1cc2cnc(Nc3ccc4c(c3)NC(=O)CC4)nc2n(C2CCCC2)c1=O] |
| 10 | 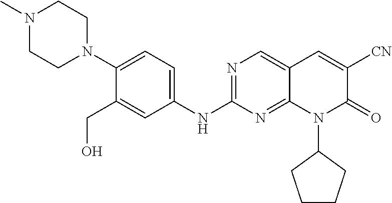 [SMILES: CN1CCN(c2ccc(Nc3ncc4cc(C#N)c(=O)n(C5CCCC5)c4n3)cc2CO)CC1] |
| 11 | 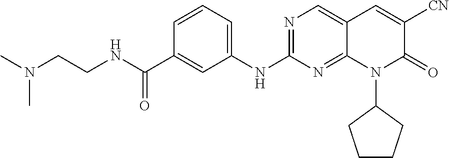 [SMILES: CN(C)CCNC(=O)c1cccc(Nc2ncc3cc(C#N)c(=O)n(C4CCCC4)c3n2)c1] |
| 12 | 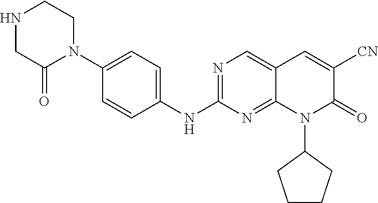 [SMILES: N#Cc1cc2cnc(Nc3ccc(N4CCNCC4=O)cc3)nc2n(C2CCCC2)c1=O] |
| 13 | 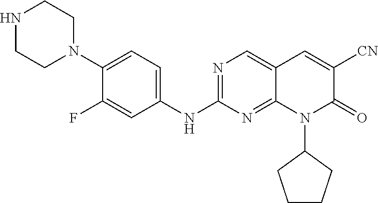 [SMILES: N#Cc1cc2cnc(Nc3ccc(N4CCNCC4)c(F)c3)nc2n(C2CCCC2)c1=O] |
| 14 | 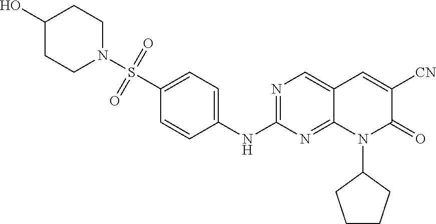 [SMILES: N#Cc1cc2cnc(Nc3ccc(S(=O)(=O)N4CCC(O)CC4)cc3)nc2n(C2CCCC2)c1=O] |
| 15 | 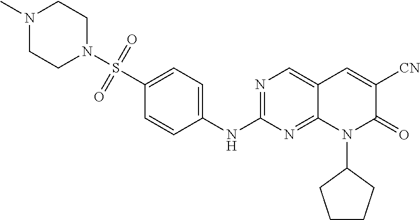 [SMILES: CN1CCN(S(=O)(=O)c2ccc(Nc3ncc4cc(C#N)c(=O)n(C5CCCC5)c4n3)cc2)CC1] |
| 16 | 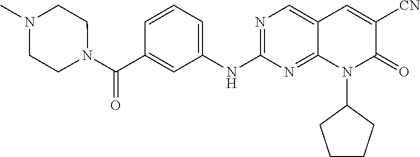 [SMILES: CN1CCN(C(=O)c2cccc(Nc3ncc4cc(C#N)c(=O)n(C5CCCC5)c4n3)c2)CC1] |
| 17 | 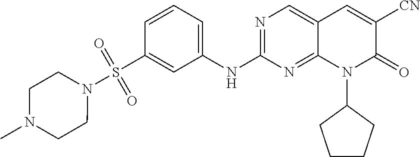 [SMILES: CN1CCN(S(=O)(=O)c2cccc(Nc3ncc4cc(C#N)c(=O)n(C5CCCC5)c4n3)c2)CC1] |
| 18 | 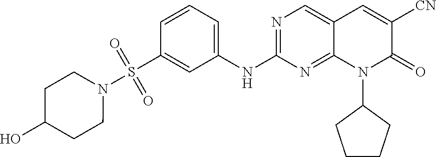 [SMILES: N#Cc1cc2cnc(Nc3cccc(S(=O)(=O)N4CCC(O)CC4)c3)nc2n(C2CCCC2)c1=O] |
| 19 | 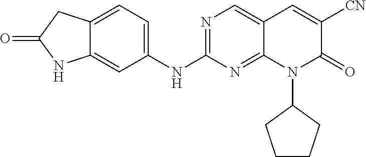 [SMILES: N#Cc1cc2cnc(Nc3ccc4c(c3)NC(=O)C4)nc2n(C2CCCC2)c1=O] |
| 20 | 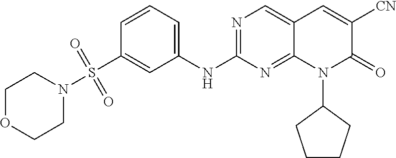 [SMILES: N#Cc1cc2cnc(Nc3cccc(S(=O)(=O)N4CCOCC4)c3)nc2n(C2CCCC2)c1=O] |
| 21 | 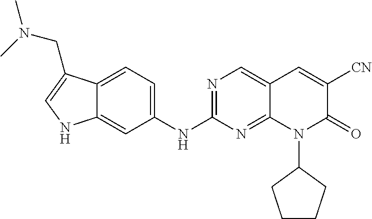 [SMILES: CN(C)Cc1c[nH]c2cc(Nc3ncc4cc(C#N)c(=O)n(C5CCCC5)c4n3)ccc12] |
| 22 | 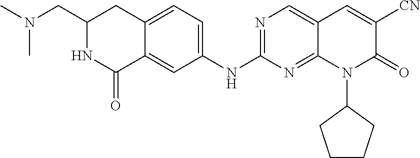 [SMILES: CN(C)CC1Cc2ccc(Nc3ncc4cc(C#N)c(=O)n(C5CCCC5)c4n3)cc2C(=O)N1] |
| 23 | 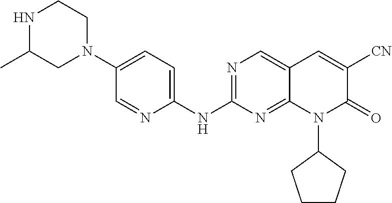 [SMILES: CC1CN(c2ccc(Nc3ncc4cc(C#N)c(=O)n(C5CCCC5)c4n3)nc2)CCN1] |
| 24 | 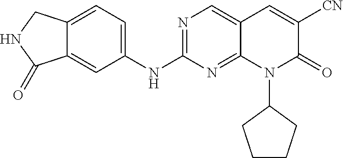 [SMILES: N#Cc1cc2cnc(Nc3ccc4c(c3)C(=O)NC4)nc2n(C2CCCC2)c1=O] |
| 25 | 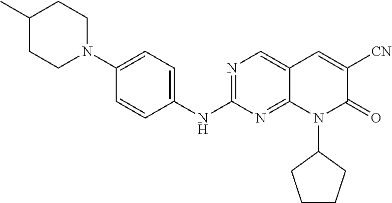 [SMILES: CC1CCN(c2ccc(Nc3ncc4cc(C#N)c(=O)n(C5CCCC5)c4n3)cc2)CC1] |
| 26 | 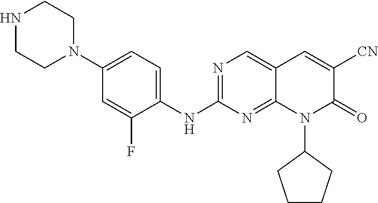 [SMILES: N#Cc1cc2cnc(Nc3ccc(N4CCNCC4)cc3F)nc2n(C2CCCC2)c1=O] |
| 27 | 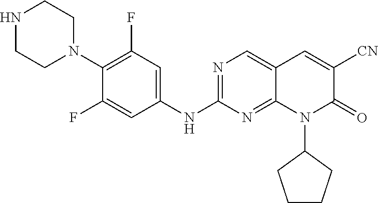 [SMILES: N#Cc1cc2cnc(Nc3cc(F)c(N4CCNCC4)c(F)c3)nc2n(C2CCCC2)c1=O] |
| 28 | 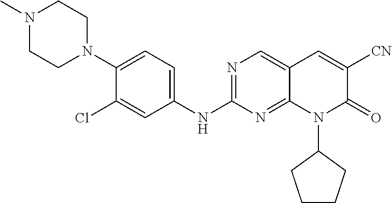 [SMILES: CN1CCN(c2ccc(Nc3ncc4cc(C#N)c(=O)n(C5CCCC5)c4n3)cc2Cl)CC1] |
| 29 | 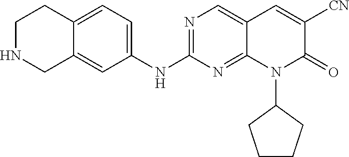 [SMILES: N#Cc1cc2cnc(Nc3ccc4c(c3)CNCC4)nc2n(C2CCCC2)c1=O] |
| 30 | 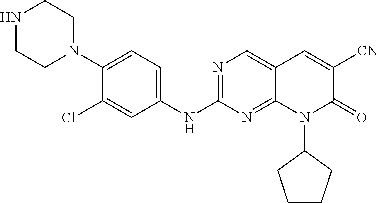 [SMILES: N#Cc1cc2cnc(Nc3ccc(N4CCNCC4)c(Cl)c3)nc2n(C2CCCC2)c1=O] |
| 31 | 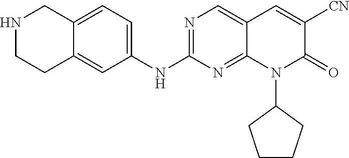 [SMILES: N#Cc1cc2cnc(Nc3ccc4c(c3)CCNC4)nc2n(C2CCCC2)c1=O] |
| 32 | 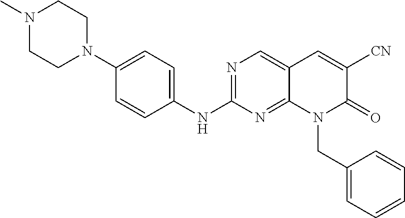 [SMILES: CN1CCN(c2ccc(Nc3ncc4cc(C#N)c(=O)n(Cc5ccccc5)c4n3)cc2)CC1] |
| 33 | 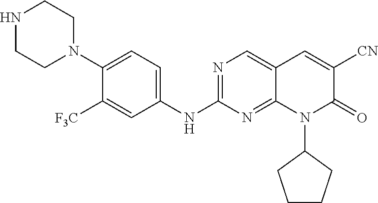 [SMILES: N#Cc1cc2cnc(Nc3ccc(N4CCNCC4)c(C(F)(F)F)c3)nc2n(C2CCCC2)c1=O] |
| 34 | 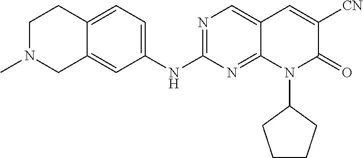 [SMILES: CN1CCc2ccc(Nc3ncc4cc(C#N)c(=O)n(C5CCCC5)c4n3)cc2C1] |
| 35 | 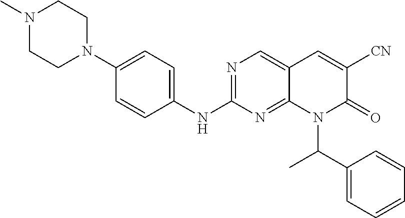 [SMILES: CC(c1ccccc1)n1c(=O)c(C#N)cc2cnc(Nc3ccc(N4CCN(C)CC4)cc3)nc21] |
| 36 | 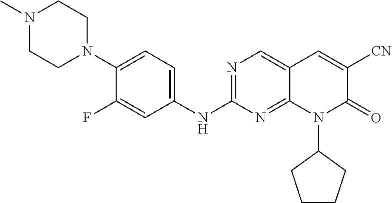 [SMILES: CN1CCN(c2ccc(Nc3ncc4cc(C#N)c(=O)n(C5CCCC5)c4n3)cc2F)CC1] |
| 37 | 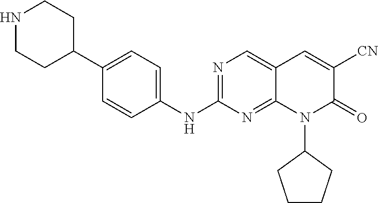 [SMILES: N#Cc1cc2cnc(Nc3ccc(C4CCNCC4)cc3)nc2n(C2CCCC2)c1=O] |
| 38 | 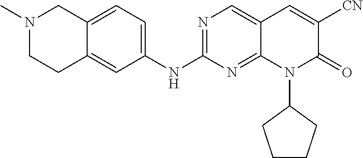 [SMILES: CN1CCc2cc(Nc3ncc4cc(C#N)c(=O)n(C5CCCC5)c4n3)ccc2C1] |
| 39 | 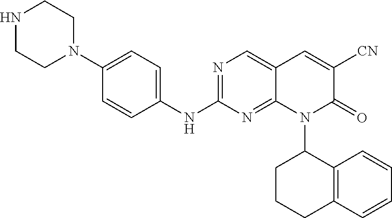 [SMILES: N#Cc1cc2cnc(Nc3ccc(N4CCNCC4)cc3)nc2n(C2CCCc3ccccc32)c1=O] |
| 40 | 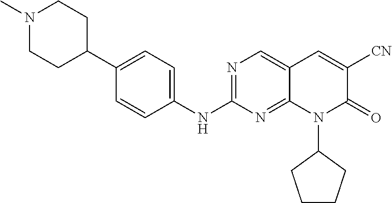 [SMILES: CN1CCC(c2ccc(Nc3ncc4cc(C#N)c(=O)n(C5CCCC5)c4n3)cc2)CC1] |
| 41 | 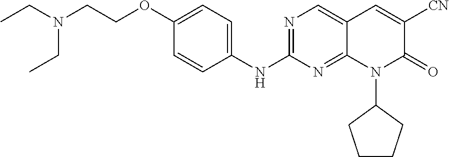 [SMILES: CCN(CC)CCOc1ccc(Nc2ncc3cc(C#N)c(=O)n(C4CCCC4)c3n2)cc1] |
| 42 | 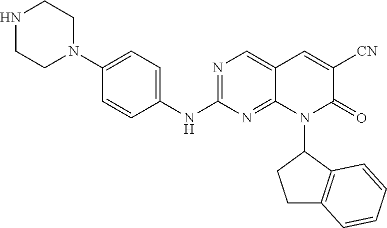 [SMILES: N#Cc1cc2cnc(Nc3ccc(N4CCNCC4)cc3)nc2n(C2CCc3ccccc32)c1=O] |
| 43 | 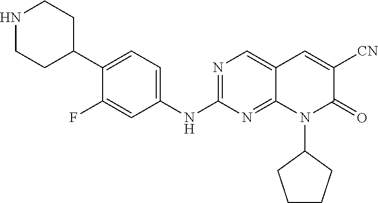 [SMILES: N#Cc1cc2cnc(Nc3ccc(C4CCNCC4)c(F)c3)nc2n(C2CCCC2)c1=O] |
| 44 | 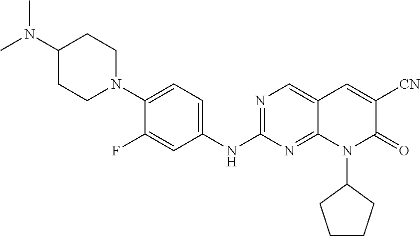 [SMILES: CN(C)C1CCN(c2ccc(Nc3ncc4cc(C#N)c(=O)n(C5CCCC5)c4n3)cc2F)CC1] |
| 45 | 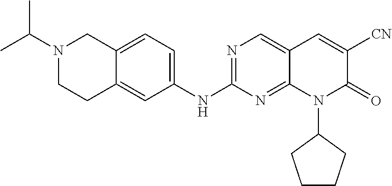 [SMILES: CC(C)N1CCc2cc(Nc3ncc4cc(C#N)c(=O)n(C5CCCC5)c4n3)ccc2C1] |
| 46 | 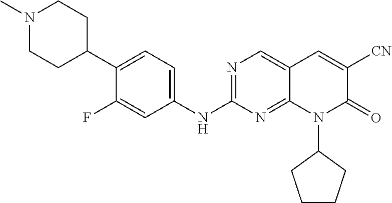 [SMILES: CN1CCC(c2ccc(Nc3ncc4cc(C#N)c(=O)n(C5CCCC5)c4n3)cc2F)CC1] |
| 47 | 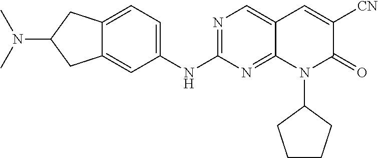 [SMILES: CN(C)C1Cc2ccc(Nc3ncc4cc(C#N)c(=O)n(C5CCCC5)c4n3)cc2C1] |
| 48 | 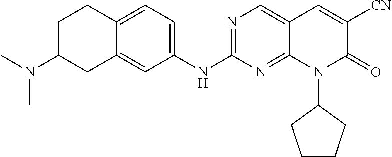 [SMILES: CN(C)C1CCc2ccc(Nc3ncc4cc(C#N)c(=O)n(C5CCCC5)c4n3)cc2C1] |
| 49 | 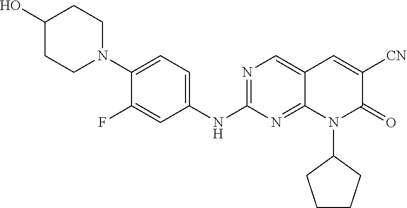 [SMILES: N#Cc1cc2cnc(Nc3ccc(N4CCC(O)CC4)c(F)c3)nc2n(C2CCCC2)c1=O] |
| 50 | 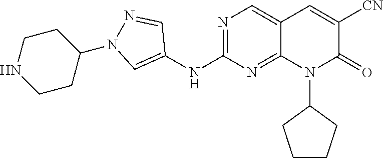 [SMILES: N#Cc1cc2cnc(Nc3cnn(C4CCNCC4)c3)nc2n(C2CCCC2)c1=O] |
| 51 | 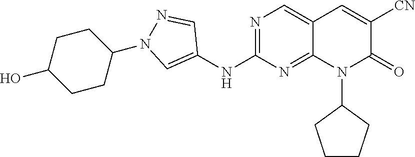 [SMILES: N#Cc1cc2cnc(Nc3cnn(C4CCC(O)CC4)c3)nc2n(C2CCCC2)c1=O] |
| 52 | 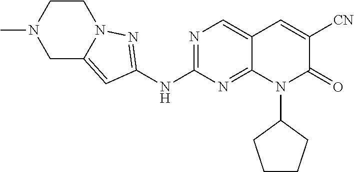 [SMILES: CN1CCn2nc(Nc3ncc4cc(C#N)c(=O)n(C5CCCC5)c4n3)cc2C1] |
| 53 | 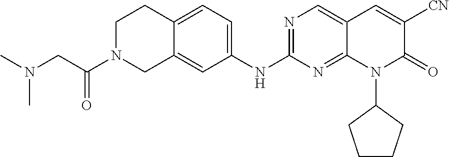 [SMILES: CN(C)CC(=O)N1CCc2ccc(Nc3ncc4cc(C#N)c(=O)n(C5CCCC5)c4n3)cc2C1] |
| 54 | 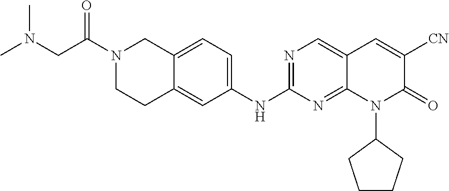 [SMILES: CN(C)CC(=O)N1CCc2cc(Nc3ncc4cc(C#N)c(=O)n(C5CCCC5)c4n3)ccc2C1] |
| 55 | 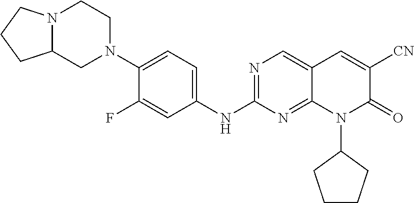 [SMILES: N#Cc1cc2cnc(Nc3ccc(N4CCN5CCCC5C4)c(F)c3)nc2n(C2CCCC2)c1=O] |
| 56 | 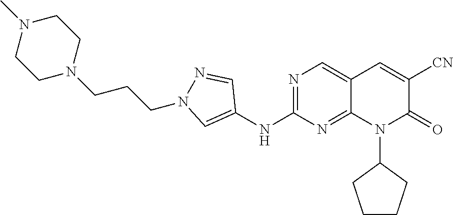 [SMILES: CN1CCN(CCCn2cc(Nc3ncc4cc(C#N)c(=O)n(C5CCCC5)c4n3)cn2)CC1] |
| 57 | 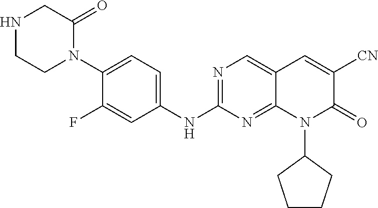 [SMILES: N#Cc1cc2cnc(Nc3ccc(N4CCNCC4=O)c(F)c3)nc2n(C2CCCC2)c1=O] |
| 58 | 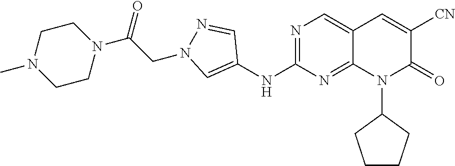 [SMILES: CN1CCN(C(=O)Cn2cc(Nc3ncc4cc(C#N)c(=O)n(C5CCCC5)c4n3)cn2)CC1] |
| 59 | 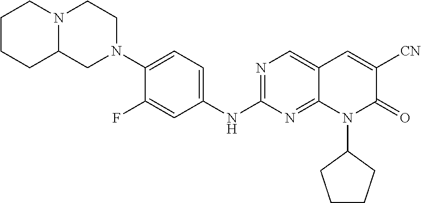 [SMILES: N#Cc1cc2cnc(Nc3ccc(N4CCN5CCCCC5C4)c(F)c3)nc2n(C2CCCC2)c1=O] |
| 60 | 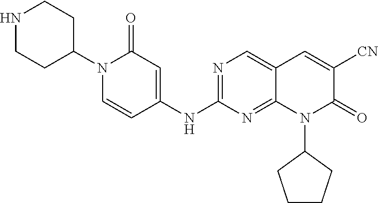 [SMILES: N#Cc1cc2cnc(Nc3ccn(C4CCNCC4)c(=O)c3)nc2n(C2CCCC2)c1=O] |
| 61 | 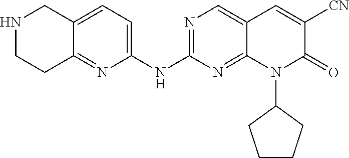 [SMILES: N#Cc1cc2cnc(Nc3ccc4c(n3)CCNC4)nc2n(C2CCCC2)c1=O] |
| 62 | 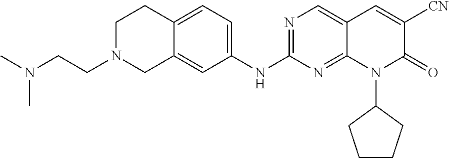 [SMILES: CN(C)CCN1CCc2ccc(Nc3ncc4cc(C#N)c(=O)n(C5CCCC5)c4n3)cc2C1] |
| 63 | 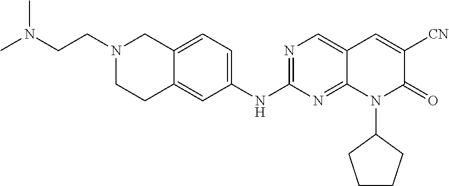 [SMILES: CN(C)CCN1CCc2cc(Nc3ncc4cc(C#N)c(=O)n(C5CCCC5)c4n3)ccc2C1] |
| 64 | 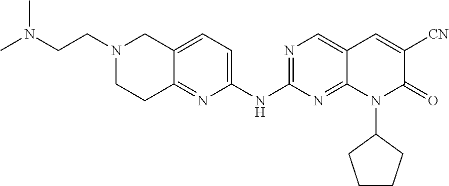 [SMILES: CN(C)CCN1CCc2nc(Nc3ncc4cc(C#N)c(=O)n(C5CCCC5)c4n3)ccc2C1] |
| 65 | 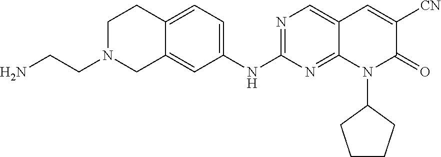 [SMILES: N#Cc1cc2cnc(Nc3ccc4c(c3)CN(CCN)CC4)nc2n(C2CCCC2)c1=O] |
| 66 | 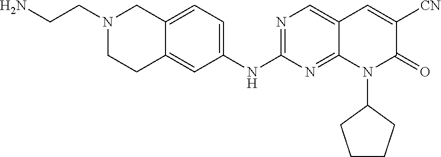 [SMILES: N#Cc1cc2cnc(Nc3ccc4c(c3)CCN(CCN)C4)nc2n(C2CCCC2)c1=O] |
| 67 | 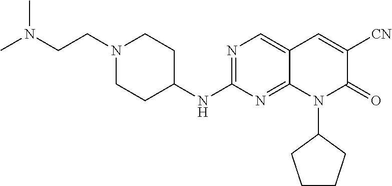 [SMILES: CN(C)CCN1CCC(Nc2ncc3cc(C#N)c(=O)n(C4CCCC4)c3n2)CC1] |
| 68 | 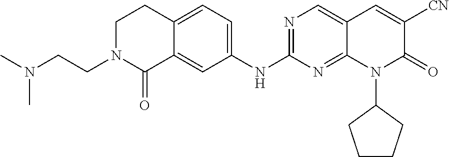 [SMILES: CN(C)CCN1CCc2ccc(Nc3ncc4cc(C#N)c(=O)n(C5CCCC5)c4n3)cc2C1=O] |
| 69 | 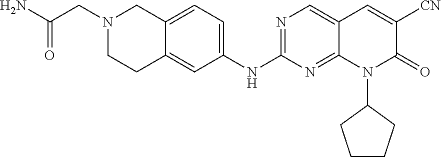 [SMILES: N#Cc1cc2cnc(Nc3ccc4c(c3)CCN(CC(N)=O)C4)nc2n(C2CCCC2)c1=O] |
| 70 | 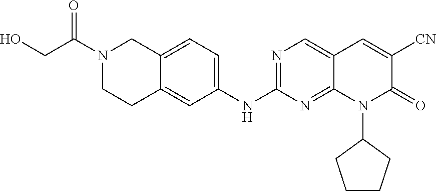 [SMILES: N#Cc1cc2cnc(Nc3ccc4c(c3)CCN(C(=O)CO)C4)nc2n(C2CCCC2)c1=O] |
| 71 | 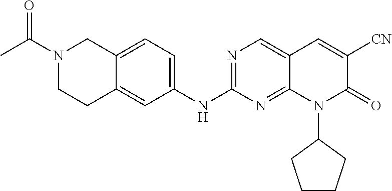 [SMILES: CC(=O)N1CCc2cc(Nc3ncc4cc(C#N)c(=O)n(C5CCCC5)c4n3)ccc2C1] |
| 72 | 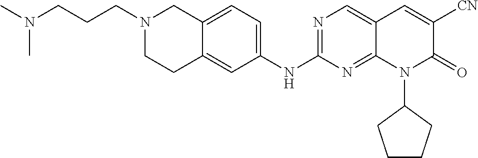 [SMILES: CN(C)CCCN1CCc2cc(Nc3ncc4cc(C#N)c(=O)n(C5CCCC5)c4n3)ccc2C1] |
| 73 | 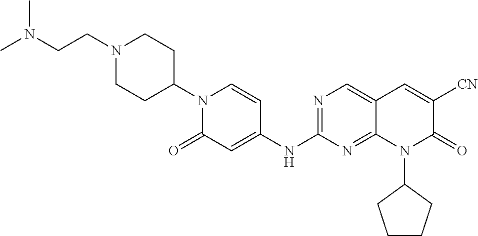 [SMILES: CN(C)CCN1CCC(n2ccc(Nc3ncc4cc(C#N)c(=O)n(C5CCCC5)c4n3)cc2=O)CC1] |
| 74 | 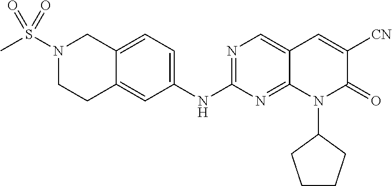 [SMILES: CS(=O)(=O)N1CCc2cc(Nc3ncc4cc(C#N)c(=O)n(C5CCCC5)c4n3)ccc2C1] |
| 75 | 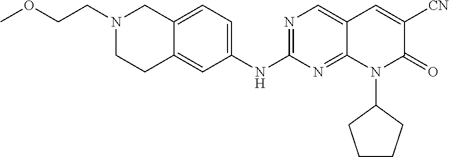 [SMILES: COCCN1CCc2cc(Nc3ncc4cc(C#N)c(=O)n(C5CCCC5)c4n3)ccc2C1] |
| 76 | 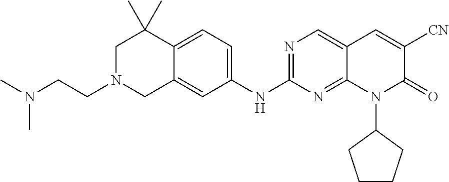 [SMILES: CN(C)CCN1Cc2cc(Nc3ncc4cc(C#N)c(=O)n(C5CCCC5)c4n3)ccc2C(C)(C)C1] |
| 77 | 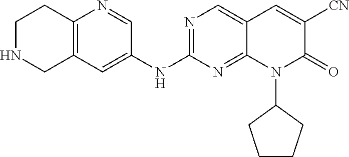 [SMILES: N#Cc1cc2cnc(Nc3cnc4c(c3)CNCC4)nc2n(C2CCCC2)c1=O] |
| 78 | 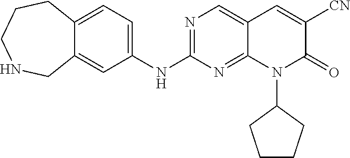 [SMILES: N#Cc1cc2cnc(Nc3ccc4c(c3)CNCCC4)nc2n(C2CCCC2)c1=O] |
| 79 | 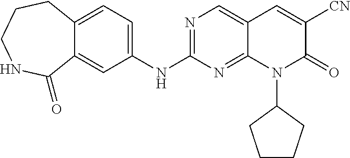 [SMILES: N#Cc1cc2cnc(Nc3ccc4c(c3)C(=O)NCCC4)nc2n(C2CCCC2)c1=O] |
| 80 | 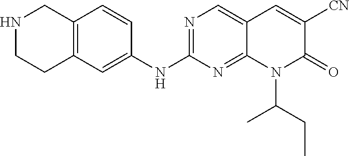 [SMILES: CCC(C)n1c(=O)c(C#N)cc2cnc(Nc3ccc4c(c3)CCNC4)nc21] |
| 81 | 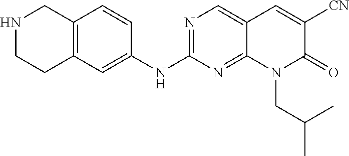 [SMILES: CC(C)Cn1c(=O)c(C#N)cc2cnc(Nc3ccc4c(c3)CCNC4)nc21] |
| 82 | 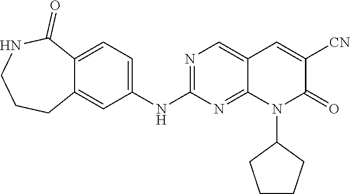 [SMILES: N#Cc1cc2cnc(Nc3ccc4c(c3)CCCNC4=O)nc2n(C2CCCC2)c1=O] |
| 83 | 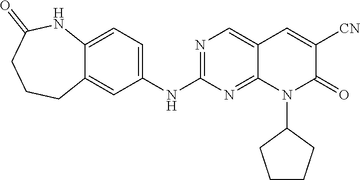 [SMILES: N#Cc1cc2cnc(Nc3ccc4c(c3)CCCC(=O)N4)nc2n(C2CCCC2)c1=O] |
| 84 | 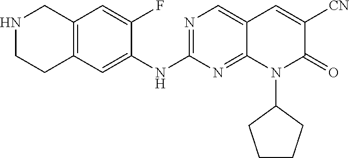 [SMILES: N#Cc1cc2cnc(Nc3cc4c(cc3F)CNCC4)nc2n(C2CCCC2)c1=O] |
| 85 | 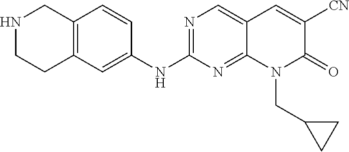 [SMILES: N#Cc1cc2cnc(Nc3ccc4c(c3)CCNC4)nc2n(CC2CC2)c1=O] |
| 86 | 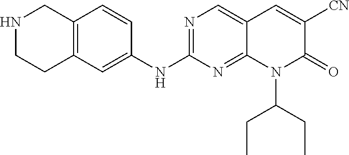 [SMILES: CCC(CC)n1c(=O)c(C#N)cc2cnc(Nc3ccc4c(c3)CCNC4)nc21] |
| 87 | 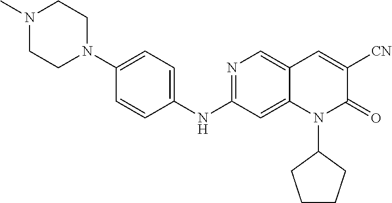 [SMILES: CN1CCN(c2ccc(Nc3cc4c(cn3)cc(C#N)c(=O)n4C3CCCC3)cc2)CC1] |
| 88 | 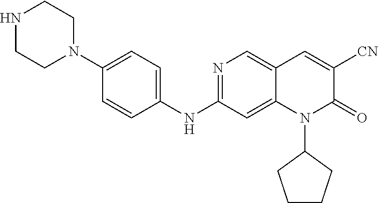 [SMILES: N#Cc1cc2cnc(Nc3ccc(N4CCNCC4)cc3)cc2n(C2CCCC2)c1=O] |
| 89 | 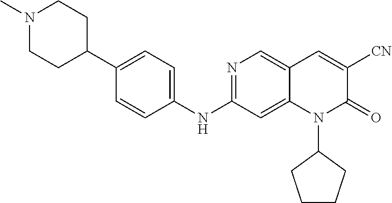 [SMILES: CN1CCC(c2ccc(Nc3cc4c(cn3)cc(C#N)c(=O)n4C3CCCC3)cc2)CC1] |
| 90 | 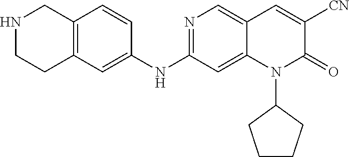 [SMILES: N#Cc1cc2cnc(Nc3ccc4c(c3)CCNC4)cc2n(C2CCCC2)c1=O] |
| 91 | 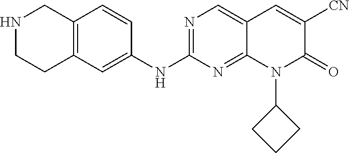 [SMILES: N#Cc1cc2cnc(Nc3ccc4c(c3)CCNC4)nc2n(C2CCC2)c1=O] |
| 92 | 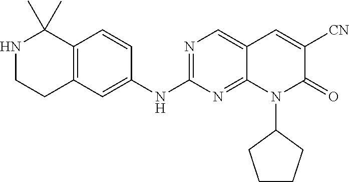 [SMILES: CC1(C)NCCc2cc(Nc3ncc4cc(C#N)c(=O)n(C5CCCC5)c4n3)ccc21] |
| 93 | 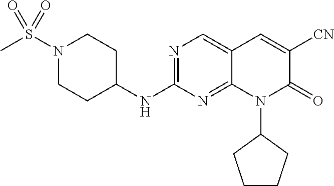 [SMILES: CS(=O)(=O)N1CCC(Nc2ncc3cc(C#N)c(=O)n(C4CCCC4)c3n2)CC1] |
| 94 | 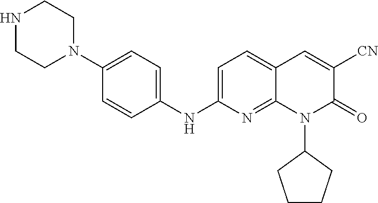 [SMILES: N#Cc1cc2ccc(Nc3ccc(N4CCNCC4)cc3)nc2n(C2CCCC2)c1=O] |
| 95 | 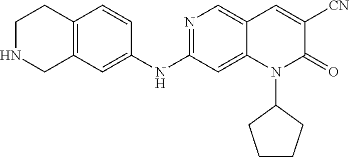 [SMILES: N#Cc1cc2cnc(Nc3ccc4c(c3)CNCC4)cc2n(C2CCCC2)c1=O] |
| 96 | 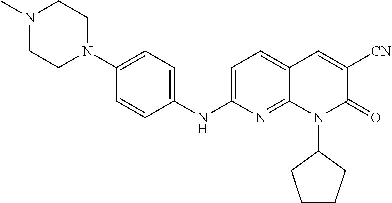 [SMILES: CN1CCN(c2ccc(Nc3ccc4cc(C#N)c(=O)n(C5CCCC5)c4n3)cc2)CC1] |
| 97 | 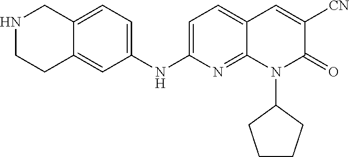 [SMILES: N#Cc1cc2ccc(Nc3ccc4c(c3)CCNC4)nc2n(C2CCCC2)c1=O] |
| 98 | 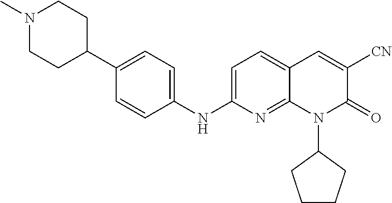 [SMILES: CN1CCC(c2ccc(Nc3ccc4cc(C#N)c(=O)n(C5CCCC5)c4n3)cc2)CC1] |
| 99 | 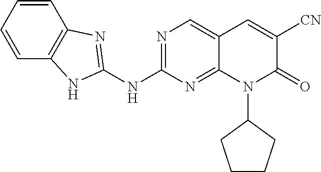 [SMILES: N#Cc1cc2cnc(Nc3nc4ccccc4[nH]3)nc2n(C2CCCC2)c1=O] |
| 100 | 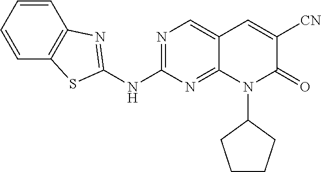 [SMILES: N#Cc1cc2cnc(Nc3nc4ccccc4s3)nc2n(C2CCCC2)c1=O] |
| 101 | 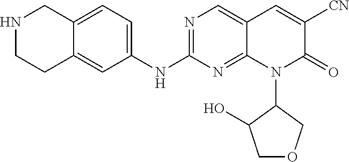 [SMILES: N#Cc1cc2cnc(Nc3ccc4c(c3)CCNC4)nc2n(C2COCC2O)c1=O] |
| 102 | 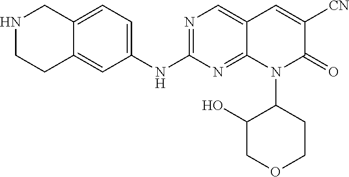 [SMILES: N#Cc1cc2cnc(Nc3ccc4c(c3)CCNC4)nc2n(C2CCOCC2O)c1=O] |
| 103 | 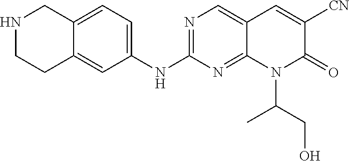 [SMILES: CC(CO)n1c(=O)c(C#N)cc2cnc(Nc3ccc4c(c3)CCNC4)nc21] |
| 104 | 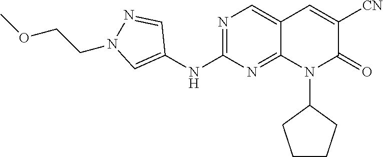 [SMILES: COCCn1cc(Nc2ncc3cc(C#N)c(=O)n(C4CCCC4)c3n2)cn1] |
| 105 | 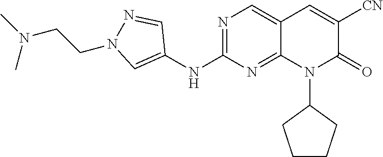 [SMILES: CN(C)CCn1cc(Nc2ncc3cc(C#N)c(=O)n(C4CCCC4)c3n2)cn1] |
| 106 | 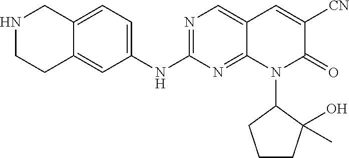 [SMILES: CC1(O)CCCC1n1c(=O)c(C#N)cc2cnc(Nc3ccc4c(c3)CCNC4)nc21] |
| 107 | 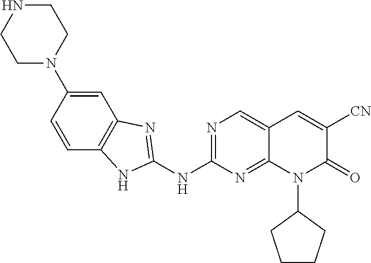 [SMILES: N#Cc1cc2cnc(Nc3nc4cc(N5CCNCC5)ccc4[nH]3)nc2n(C2CCCC2)c1=O] |
| 108 | 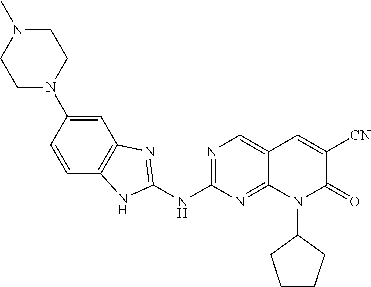 [SMILES: CN1CCN(c2ccc3[nH]c(Nc4ncc5cc(C#N)c(=O)n(C6CCCC6)c5n4)nc3c2)CC1] |
| 109 | 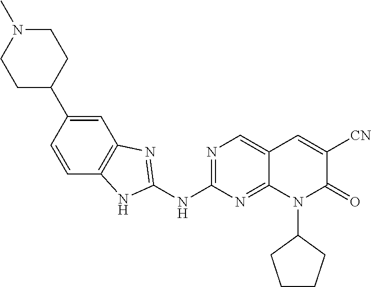 [SMILES: CN1CCC(c2ccc3[nH]c(Nc4ncc5cc(C#N)c(=O)n(C6CCCC6)c5n4)nc3c2)CC1] |
| 110 | 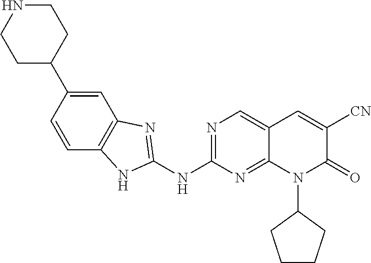 [SMILES: N#Cc1cc2cnc(Nc3nc4cc(C5CCNCC5)ccc4[nH]3)nc2n(C2CCCC2)c1=O] |
| 111 | 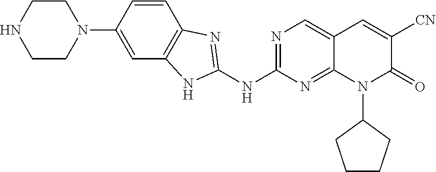 [SMILES: N#Cc1cc2cnc(Nc3nc4ccc(N5CCNCC5)cc4[nH]3)nc2n(C2CCCC2)c1=O] |
| 112 | 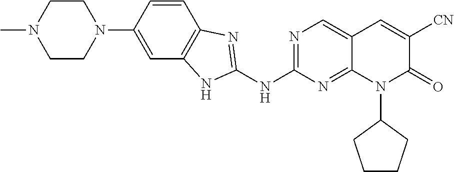 [SMILES: CN1CCN(c2ccc3nc(Nc4ncc5cc(C#N)c(=O)n(C6CCCC6)c5n4)[nH]c3c2)CC1] |
| 113 | 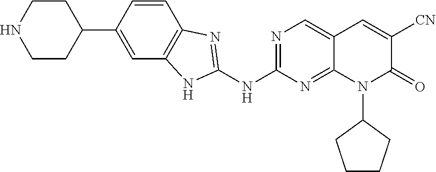 [SMILES: N#Cc1cc2cnc(Nc3nc4ccc(C5CCNCC5)cc4[nH]3)nc2n(C2CCCC2)c1=O] |
| 114 | 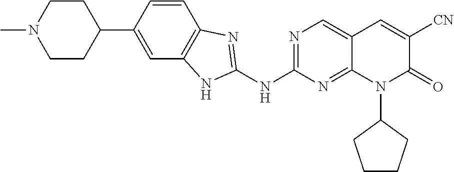 [SMILES: CN1CCC(c2ccc3nc(Nc4ncc5cc(C#N)c(=O)n(C6CCCC6)c5n4)[nH]c3c2)CC1] |
| 115 | 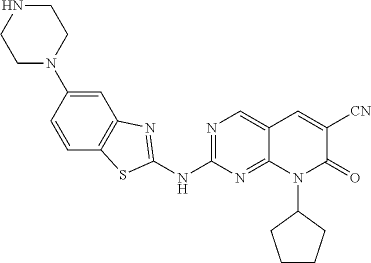 [SMILES: N#Cc1cc2cnc(Nc3nc4cc(N5CCNCC5)ccc4s3)nc2n(C2CCCC2)c1=O] |
| 116 | 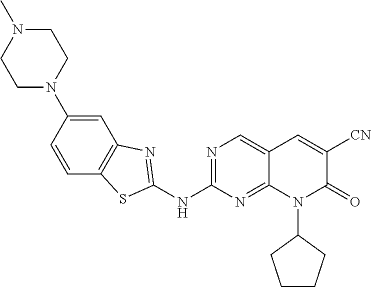 [SMILES: CN1CCN(c2ccc3sc(Nc4ncc5cc(C#N)c(=O)n(C6CCCC6)c5n4)nc3c2)CC1] |
| 117 | 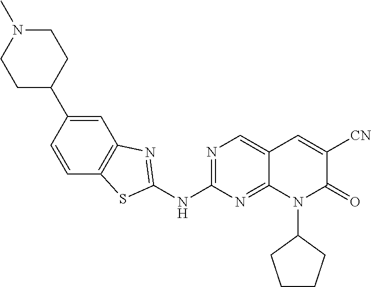 [SMILES: CN1CCC(c2ccc3sc(Nc4ncc5cc(C#N)c(=O)n(C6CCCC6)c5n4)nc3c2)CC1] |
| 118 | 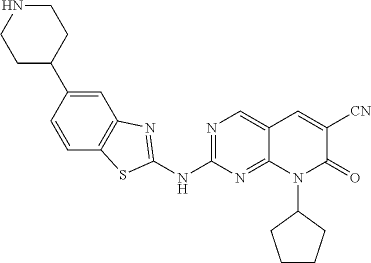 [SMILES: N#Cc1cc2cnc(Nc3nc4cc(C5CCNCC5)ccc4s3)nc2n(C2CCCC2)c1=O] |
| 119 | 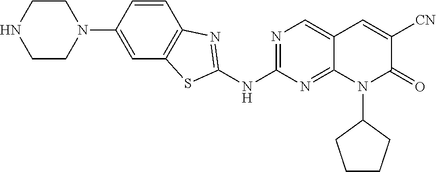 [SMILES: N#Cc1cc2cnc(Nc3nc4ccc(N5CCNCC5)cc4s3)nc2n(C2CCCC2)c1=O] |
| 120 | 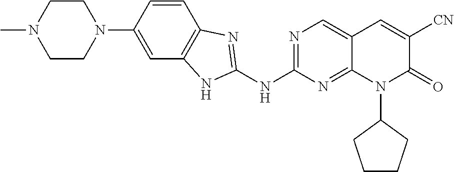 [SMILES: CN1CCN(c2ccc3nc(Nc4ncc5cc(C#N)c(=O)n(C6CCCC6)c5n4)[nH]c3c2)CC1] |
| 121 | 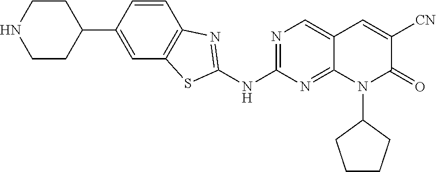 [SMILES: N#Cc1cc2cnc(Nc3nc4ccc(C5CCNCC5)cc4s3)nc2n(C2CCCC2)c1=O] |
| 122 | 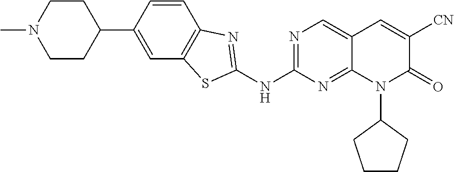 [SMILES: CN1CCC(c2ccc3nc(Nc4ncc5cc(C#N)c(=O)n(C6CCCC6)c5n4)sc3c2)CC1] |
| 123 | 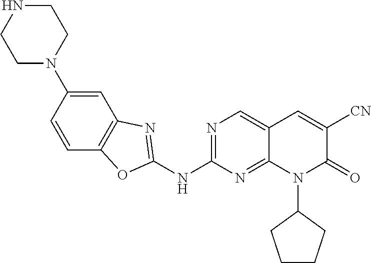 [SMILES: N#Cc1cc2cnc(Nc3nc4cc(N5CCNCC5)ccc4o3)nc2n(C2CCCC2)c1=O] |
| 124 | 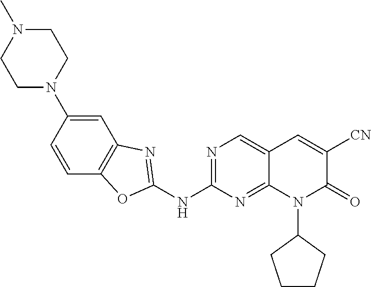 [SMILES: CN1CCN(c2ccc3oc(Nc4ncc5cc(C#N)c(=O)n(C6CCCC6)c5n4)nc3c2)CC1] |
| 125 | 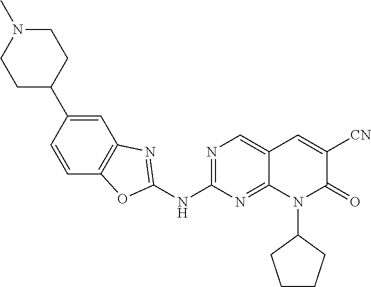 [SMILES: CN1CCC(c2ccc3oc(Nc4ncc5cc(C#N)c(=O)n(C6CCCC6)c5n4)nc3c2)CC1] |
| 126 | 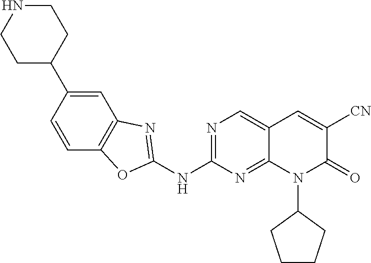 [SMILES: N#Cc1cc2cnc(Nc3nc4cc(C5CCNCC5)ccc4o3)nc2n(C2CCCC2)c1=O] |
| 127 | 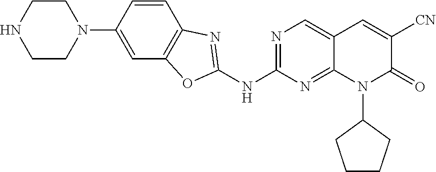 [SMILES: N#Cc1cc2cnc(Nc3nc4ccc(N5CCNCC5)cc4o3)nc2n(C2CCCC2)c1=O] |
| 128 | 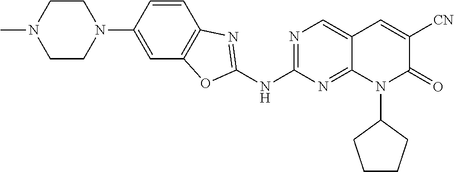 [SMILES: CN1CCN(c2ccc3nc(Nc4ncc5cc(C#N)c(=O)n(C6CCCC6)c5n4)oc3c2)CC1] |
| 129 | 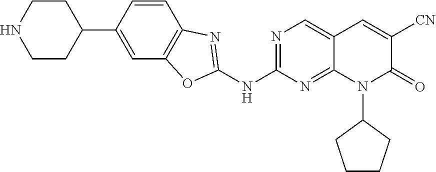 [SMILES: N#Cc1cc2cnc(Nc3nc4ccc(C5CCNCC5)cc4o3)nc2n(C2CCCC2)c1=O] |
| 130 | 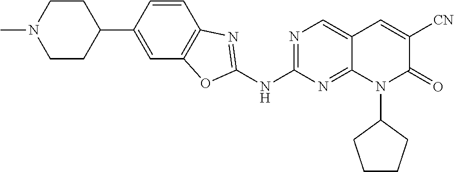 [SMILES: CN1CCC(c2ccc3nc(Nc4ncc5cc(C#N)c(=O)n(C6CCCC6)c5n4)oc3c2)CC1] |
| 131 | 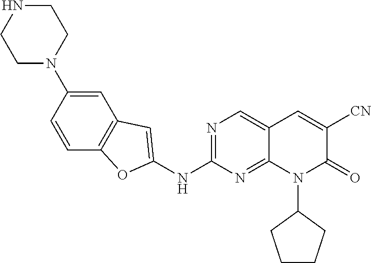 [SMILES: N#Cc1cc2cnc(Nc3cc4cc(N5CCNCC5)ccc4o3)nc2n(C2CCCC2)c1=O] |
| 132 | 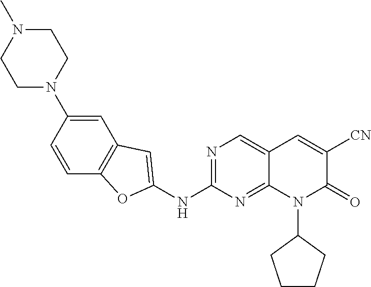 [SMILES: CN1CCN(c2ccc3oc(Nc4ncc5cc(C#N)c(=O)n(C6CCCC6)c5n4)cc3c2)CC1] |
| 133 | 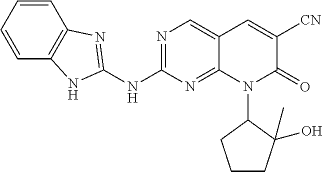 [SMILES: CC1(O)CCCC1n1c(=O)c(C#N)cc2cnc(Nc3nc4ccccc4[nH]3)nc21] |
| 134 | 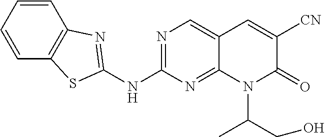 [SMILES: CC(CO)n1c(=O)c(C#N)cc2cnc(Nc3nc4ccccc4s3)nc21] |
| 135 | 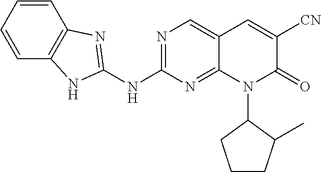 [SMILES: CC1CCCC1n1c(=O)c(C#N)cc2cnc(Nc3nc4ccccc4[nH]3)nc21] |
| 136 | 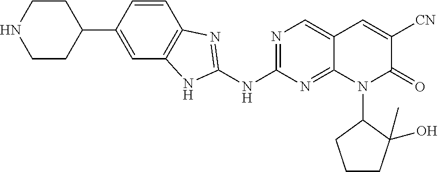 [SMILES: CC1(O)CCCC1n1c(=O)c(C#N)cc2cnc(Nc3nc4ccc(C5CCNCC5)cc4[nH]3)nc21] |
| 137 | 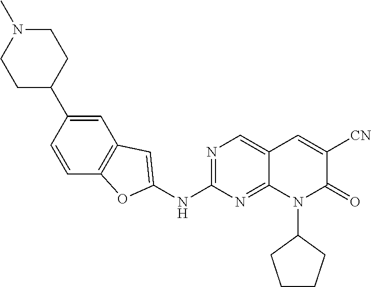 [SMILES: CN1CCC(c2ccc3oc(Nc4ncc5cc(C#N)c(=O)n(C6CCCC6)c5n4)cc3c2)CC1] |
| 138 | 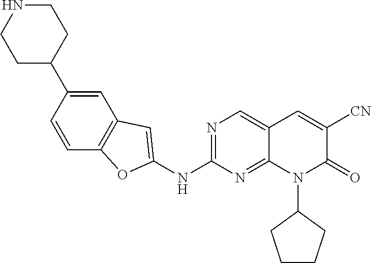 [SMILES: N#Cc1cc2cnc(Nc3cc4cc(C5CCNCC5)ccc4o3)nc2n(C2CCCC2)c1=O] |
| 139 | 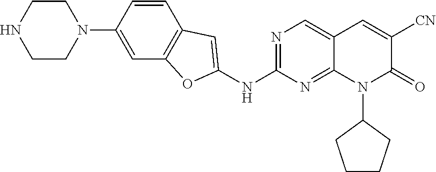 [SMILES: N#Cc1cc2cnc(Nc3cc4ccc(N5CCNCC5)cc4o3)nc2n(C2CCCC2)c1=O] |
| 140 | 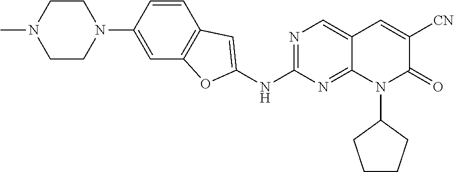 [SMILES: CN1CCN(c2ccc3cc(Nc4ncc5cc(C#N)c(=O)n(C6CCCC6)c5n4)oc3c2)CC1] |
| 141 | 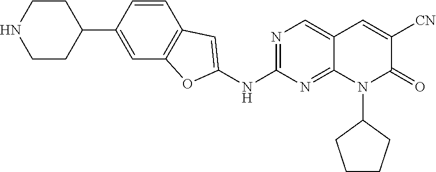 [SMILES: N#Cc1cc2cnc(Nc3cc4ccc(C5CCNCC5)cc4o3)nc2n(C2CCCC2)c1=O] |
| 142 | 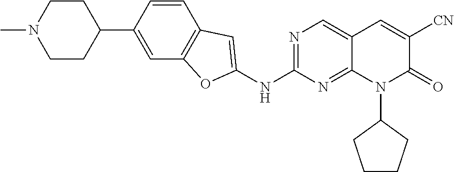 [SMILES: CN1CCC(c2ccc3cc(Nc4ncc5cc(C#N)c(=O)n(C6CCCC6)c5n4)oc3c2)CC1] |
| 143 | 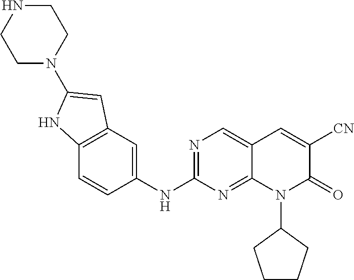 [SMILES: N#Cc1cc2cnc(Nc3ccc4[nH]c(N5CCNCC5)cc4c3)nc2n(C2CCCC2)c1=O] |
| 144 | 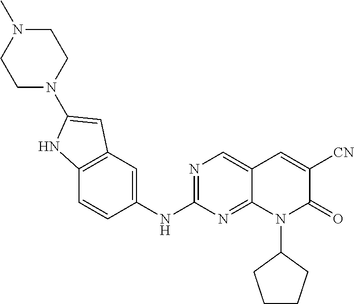 [SMILES: CN1CCN(c2cc3cc(Nc4ncc5cc(C#N)c(=O)n(C6CCCC6)c5n4)ccc3[nH]2)CC1] |
| 145 | 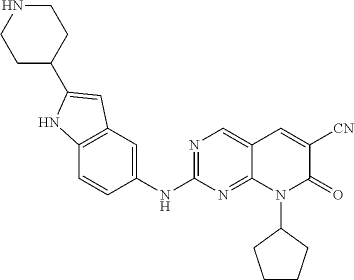 [SMILES: N#Cc1cc2cnc(Nc3ccc4[nH]c(C5CCNCC5)cc4c3)nc2n(C2CCCC2)c1=O] |
| 146 | 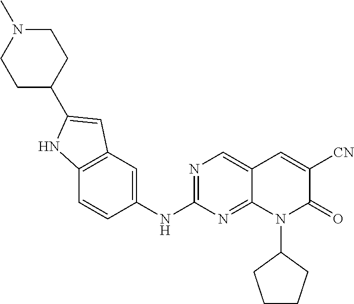 [SMILES: CN1CCC(c2cc3cc(Nc4ncc5cc(C#N)c(=O)n(C6CCCC6)c5n4)ccc3[nH]2)CC1] |
| 147 | 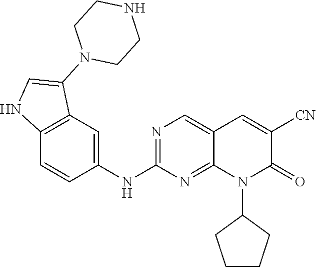 [SMILES: N#Cc1cc2cnc(Nc3ccc4[nH]cc(N5CCNCC5)c4c3)nc2n(C2CCCC2)c1=O] |
| 148 | 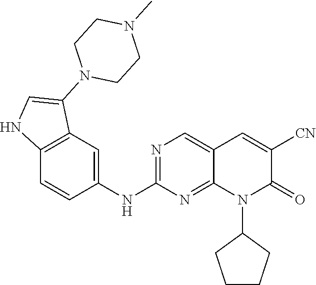 [SMILES: CN1CCN(c2c[nH]c3ccc(Nc4ncc5cc(C#N)c(=O)n(C6CCCC6)c5n4)cc23)CC1] |
| 149 | 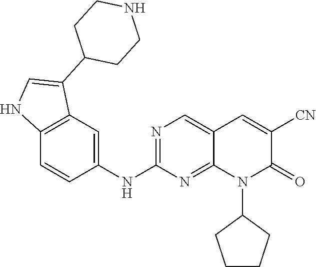 [SMILES: N#Cc1cc2cnc(Nc3ccc4[nH]cc(C5CCNCC5)c4c3)nc2n(C2CCCC2)c1=O] |
| 150 | 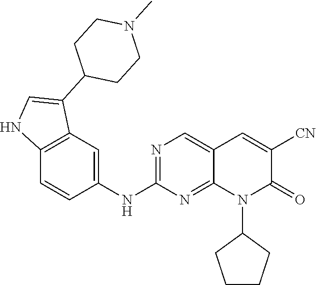 [SMILES: CN1CCC(c2c[nH]c3ccc(Nc4ncc5cc(C#N)c(=O)n(C6CCCC6)c5n4)cc23)CC1] |
| 151 | 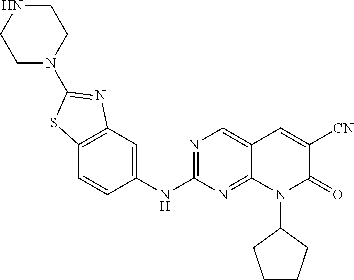 [SMILES: N#Cc1cc2cnc(Nc3ccc4sc(N5CCNCC5)nc4c3)nc2n(C2CCCC2)c1=O] |
| 152 | 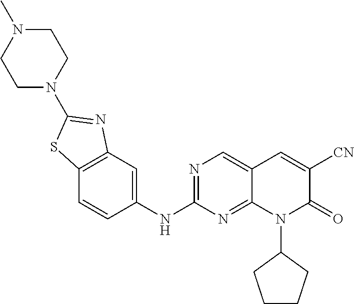 [SMILES: CN1CCN(c2nc3cc(Nc4ncc5cc(C#N)c(=O)n(C6CCCC6)c5n4)ccc3s2)CC1] |
| 153 | 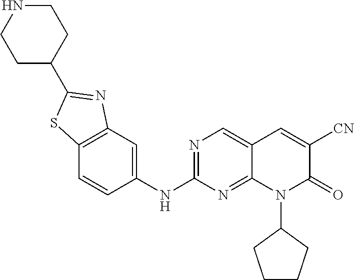 [SMILES: N#Cc1cc2cnc(Nc3ccc4sc(C5CCNCC5)nc4c3)nc2n(C2CCCC2)c1=O] |
| 154 | 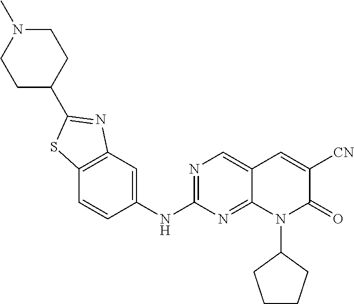 [SMILES: CN1CCC(c2nc3cc(Nc4ncc5cc(C#N)c(=O)n(C6CCCC6)c5n4)ccc3s2)CC1] |
| 155 | 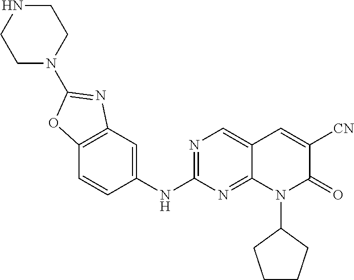 [SMILES: N#Cc1cc2cnc(Nc3ccc4oc(N5CCNCC5)nc4c3)nc2n(C2CCCC2)c1=O] |
| 156 | 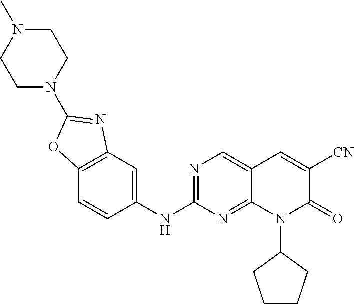 [SMILES: CN1CCN(c2nc3cc(Nc4ncc5cc(C#N)c(=O)n(C6CCCC6)c5n4)ccc3o2)CC1] |
| 157 | 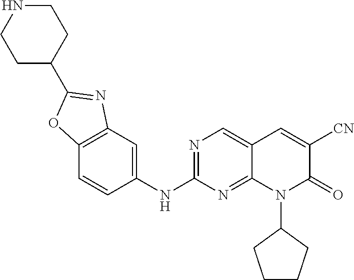 [SMILES: N#Cc1cc2cnc(Nc3ccc4oc(C5CCNCC5)nc4c3)nc2n(C2CCCC2)c1=O] |
| 158 | 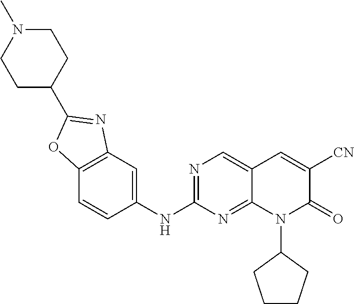 [SMILES: CN1CCC(c2nc3cc(Nc4ncc5cc(C#N)c(=O)n(C6CCCC6)c5n4)ccc3o2)CC1] |
| 159 | 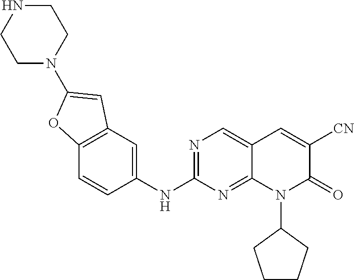 [SMILES: N#Cc1cc2cnc(Nc3ccc4oc(N5CCNCC5)cc4c3)nc2n(C2CCCC2)c1=O] |
| 160 | 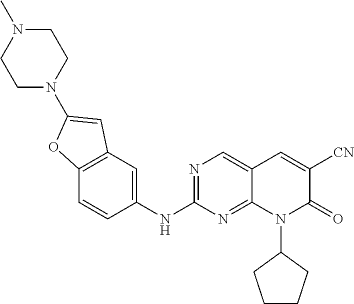 [SMILES: CN1CCN(c2cc3cc(Nc4ncc5cc(C#N)c(=O)n(C6CCCC6)c5n4)ccc3o2)CC1] |
| 161 | 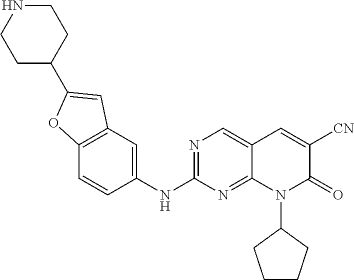 [SMILES: N#Cc1cc2cnc(Nc3ccc4oc(C5CCNCC5)cc4c3)nc2n(C2CCCC2)c1=O] |
| 162 | 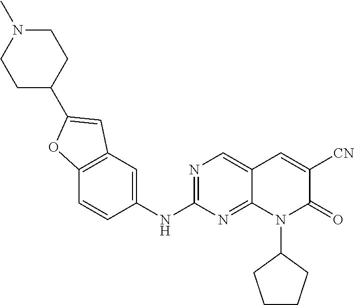 [SMILES: CN1CCC(c2cc3cc(Nc4ncc5cc(C#N)c(=O)n(C6CCCC6)c5n4)ccc3o2)CC1] |
| 163 | 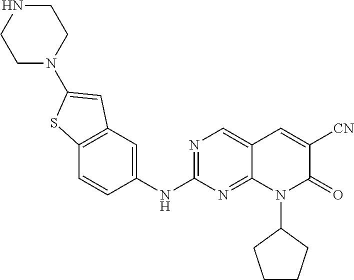 [SMILES: N#Cc1cc2cnc(Nc3ccc4sc(N5CCNCC5)cc4c3)nc2n(C2CCCC2)c1=O] |
| 164 | 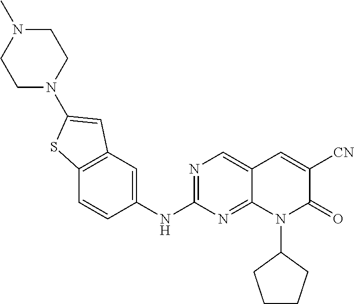 [SMILES: CN1CCN(c2cc3cc(Nc4ncc5cc(C#N)c(=O)n(C6CCCC6)c5n4)ccc3s2)CC1] |
| 165 | 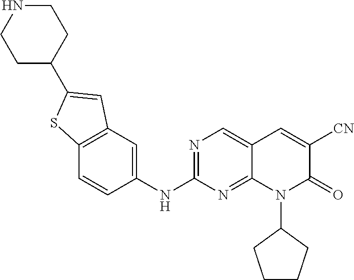 [SMILES: N#Cc1cc2cnc(Nc3ccc4sc(C5CCNCC5)cc4c3)nc2n(C2CCCC2)c1=O] |
| 166 | 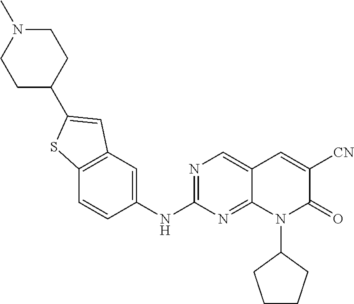 [SMILES: CN1CCC(c2cc3cc(Nc4ncc5cc(C#N)c(=O)n(C6CCCC6)c5n4)ccc3s2)CC1] |
| 167 | 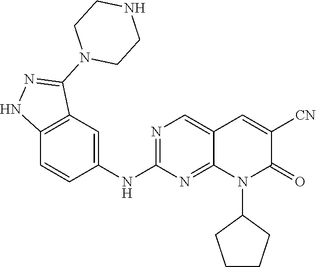 [SMILES: N#Cc1cc2cnc(Nc3ccc4[nH]nc(N5CCNCC5)c4c3)nc2n(C2CCCC2)c1=O] |
| 168 | 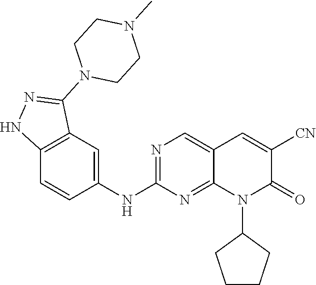 [SMILES: CN1CCN(c2n[nH]c3ccc(Nc4ncc5cc(C#N)c(=O)n(C6CCCC6)c5n4)cc23)CC1] |
| 169 | 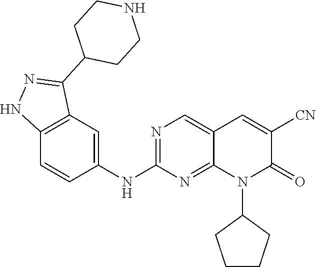 [SMILES: N#Cc1cc2cnc(Nc3ccc4[nH]nc(C5CCNCC5)c4c3)nc2n(C2CCCC2)c1=O] |
| 170 | 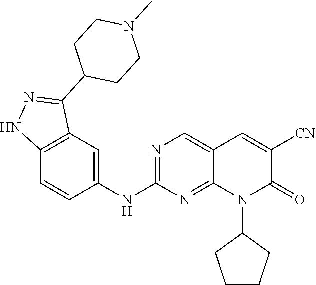 [SMILES: CN1CCC(c2n[nH]c3ccc(Nc4ncc5cc(C#N)c(=O)n(C6CCCC6)c5n4)cc23)CC1] |
| 171 | 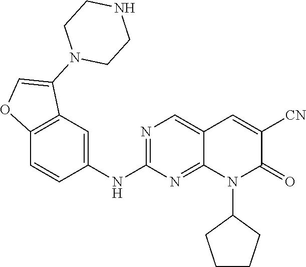 [SMILES: N#Cc1cc2cnc(Nc3ccc4occ(N5CCNCC5)c4c3)nc2n(C2CCCC2)c1=O] |
| 172 | 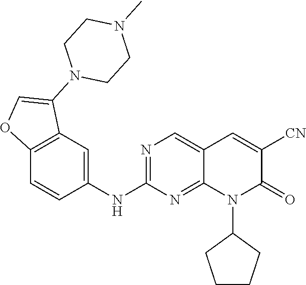 [SMILES: CN1CCN(c2coc3ccc(Nc4ncc5cc(C#N)c(=O)n(C6CCCC6)c5n4)cc23)CC1] |
| 173 | 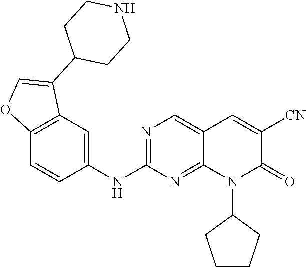 [SMILES: N#Cc1cc2cnc(Nc3ccc4occ(C5CCNCC5)c4c3)nc2n(C2CCCC2)c1=O] |
| 174 | 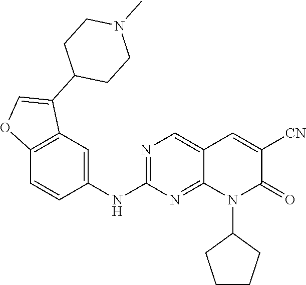 [SMILES: CN1CCC(c2coc3ccc(Nc4ncc5cc(C#N)c(=O)n(C6CCCC6)c5n4)cc23)CC1] |
| 175 | 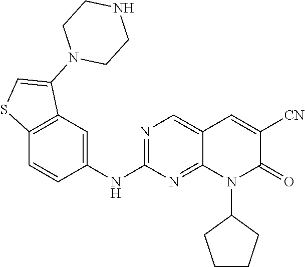 [SMILES: N#Cc1cc2cnc(Nc3ccc4scc(N5CCNCC5)c4c3)nc2n(C2CCCC2)c1=O] |
| 176 | 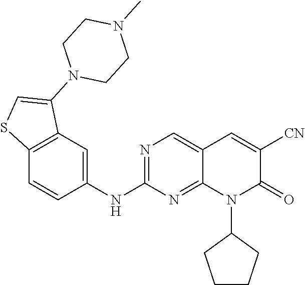 [SMILES: CN1CCN(c2csc3ccc(Nc4ncc5cc(C#N)c(=O)n(C6CCCC6)c5n4)cc23)CC1] |
| 177 | 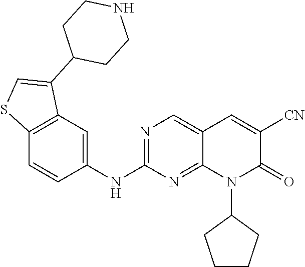 [SMILES: N#Cc1cc2cnc(Nc3ccc4scc(C5CCNCC5)c4c3)nc2n(C2CCCC2)c1=O] |
| 178 | 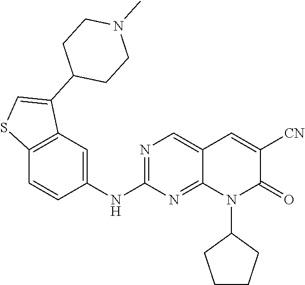 [SMILES: CN1CCC(c2csc3ccc(Nc4ncc5cc(C#N)c(=O)n(C6CCCC6)c5n4)cc23)CC1] |
| 179 | 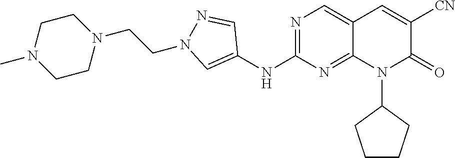 [SMILES: CN1CCN(CCn2cc(Nc3ncc4cc(C#N)c(=O)n(C5CCCC5)c4n3)cn2)CC1] |
| 180 | 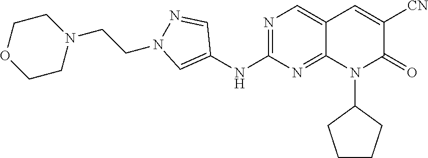 [SMILES: N#Cc1cc2cnc(Nc3cnn(CCN4CCOCC4)c3)nc2n(C2CCCC2)c1=O] |
| 181 | 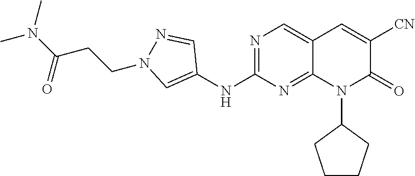 [SMILES: CN(C)C(=O)CCn1cc(Nc2ncc3cc(C#N)c(=O)n(C4CCCC4)c3n2)cn1] |
| 182 | 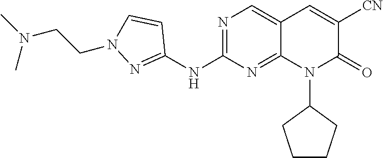 [SMILES: CN(C)CCn1ccc(Nc2ncc3cc(C#N)c(=O)n(C4CCCC4)c3n2)n1] |
| 183 | 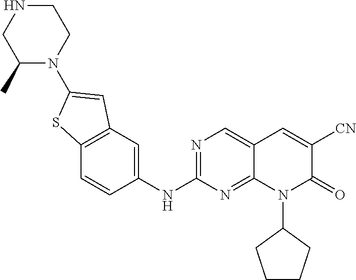 [SMILES: C[C@H]1CNCCN1c1cc2cc(Nc3ncc4cc(C#N)c(=O)n(C5CCCC5)c4n3)ccc2s1] |
| 184 | 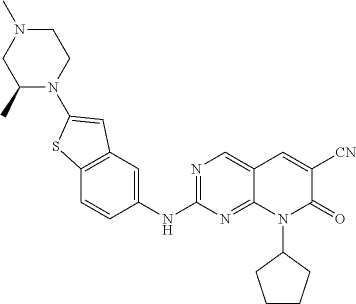 [SMILES: C[C@H]1CN(C)CCN1c1cc2cc(Nc3ncc4cc(C#N)c(=O)n(C5CCCC5)c4n3)ccc2s1] |
| 185 | 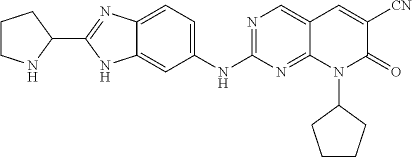 [SMILES: N#Cc1cc2cnc(Nc3ccc4nc(C5CCCN5)[nH]c4c3)nc2n(C2CCCC2)c1=O] |
| 186 | 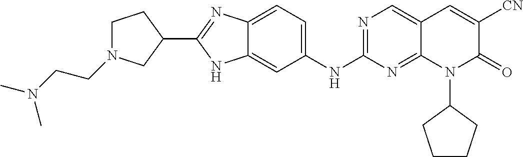 [SMILES: CN(C)CCN1CCC(c2nc3ccc(Nc4ncc5cc(C#N)c(=O)n(C6CCCC6)c5n4)cc3[nH]2)C1] |
| 187 | 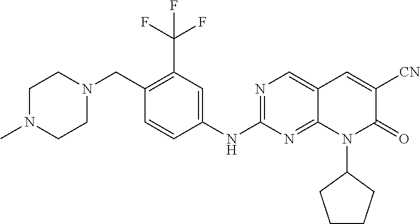 [SMILES: CN1CCN(Cc2ccc(Nc3ncc4cc(C#N)c(=O)n(C5CCCC5)c4n3)cc2C(F)(F)F)CC1] |
| 188 | 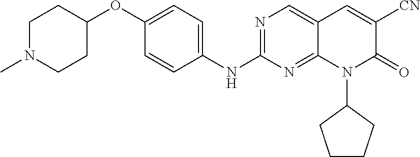 [SMILES: CN1CCC(Oc2ccc(Nc3ncc4cc(C#N)c(=O)n(C5CCCC5)c4n3)cc2)CC1] |
| 189 | 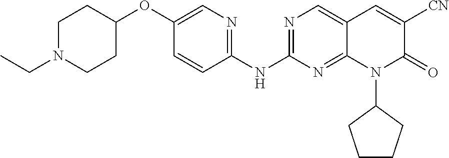 [SMILES: CCN1CCC(Oc2ccc(Nc3ncc4cc(C#N)c(=O)n(C5CCCC5)c4n3)nc2)CC1] |
| 190 | 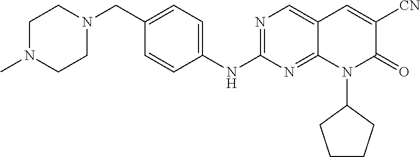 [SMILES: CN1CCN(Cc2ccc(Nc3ncc4cc(C#N)c(=O)n(C5CCCC5)c4n3)cc2)CC1] |
| 191 | 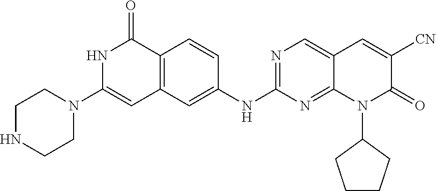 [SMILES: N#Cc1cc2cnc(Nc3ccc4c(=O)[nH]c(N5CCNCC5)cc4c3)nc2n(C2CCCC2)c1=O] |
| 192 | 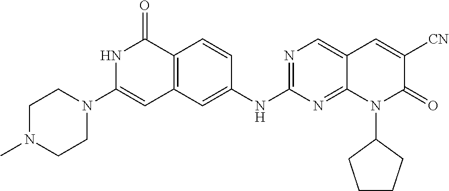 [SMILES: CN1CCN(c2cc3cc(Nc4ncc5cc(C#N)c(=O)n(C6CCCC6)c5n4)ccc3c(=O)[nH]2)CC1] |
| 193 | 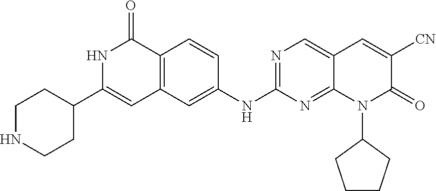 [SMILES: N#Cc1cc2cnc(Nc3ccc4c(=O)[nH]c(C5CCNCC5)cc4c3)nc2n(C2CCCC2)c1=O] |
| 194 | 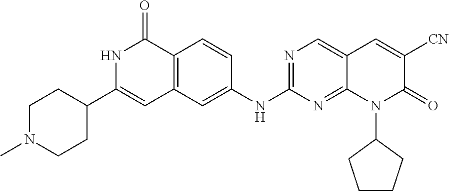 [SMILES: CN1CCC(c2cc3cc(Nc4ncc5cc(C#N)c(=O)n(C6CCCC6)c5n4)ccc3c(=O)[nH]2)CC1] |
| 195 | 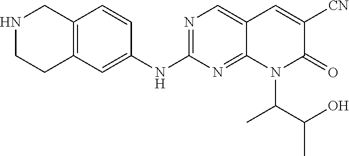 [SMILES: CC(O)C(C)n1c(=O)c(C#N)cc2cnc(Nc3ccc4c(c3)CCNC4)nc21] |
| 196 | 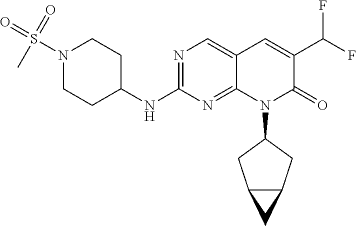 [SMILES: CS(=O)(=O)N1CCC(Nc2ncc3cc(C(F)F)c(=O)n([C@H]4C[C@H]5C[C@H]5C4)c3n2)CC1] |
| 197 | 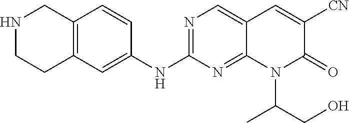 [SMILES: CC(CO)n1c(=O)c(C#N)cc2cnc(Nc3ccc4c(c3)CCNC4)nc21] |
| 198 | 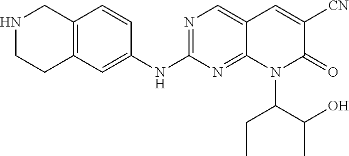 [SMILES: CCC(C(C)O)n1c(=O)c(C#N)cc2cnc(Nc3ccc4c(c3)CCNC4)nc21] |
| 199 | 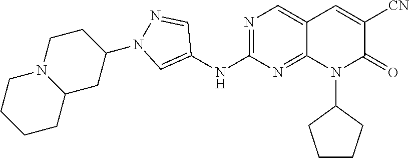 [SMILES: N#Cc1cc2cnc(Nc3cnn(C4CCN5CCCCC5C4)c3)nc2n(C2CCCC2)c1=O] |
| 200 | 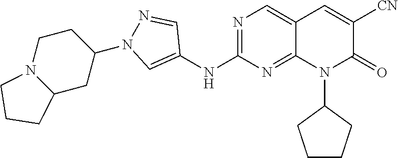 [SMILES: N#Cc1cc2cnc(Nc3cnn(C4CCN5CCCC5C4)c3)nc2n(C2CCCC2)c1=O] |
| 201 | 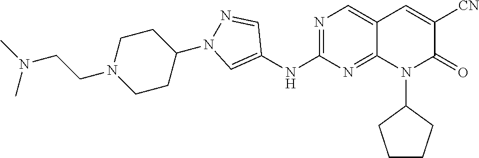 [SMILES: CN(C)CCN1CCC(n2cc(Nc3ncc4cc(C#N)c(=O)n(C5CCCC5)c4n3)cn2)CC1] |
| 202 | 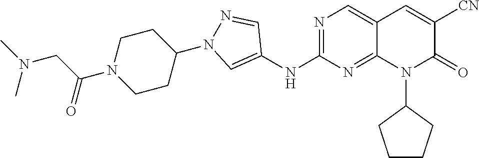 [SMILES: CN(C)CC(=O)N1CCC(n2cc(Nc3ncc4cc(C#N)c(=O)n(C5CCCC5)c4n3)cn2)CC1] |
| 203 | 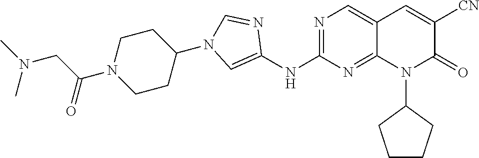 [SMILES: CN(C)CC(=O)N1CCC(n2cnc(Nc3ncc4cc(C#N)c(=O)n(C5CCCC5)c4n3)c2)CC1] |
| 204 | 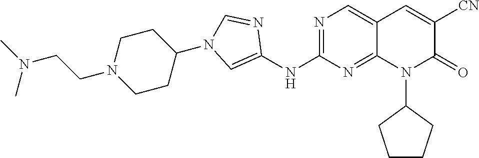 [SMILES: CN(C)CCN1CCC(n2cnc(Nc3ncc4cc(C#N)c(=O)n(C5CCCC5)c4n3)c2)CC1] |
| 205 | 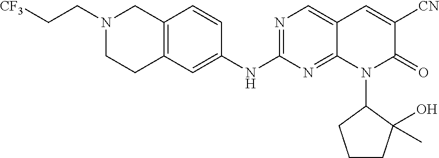 [SMILES: CC1(O)CCCC1n1c(=O)c(C#N)cc2cnc(Nc3ccc4c(c3)CCN(CCC(F)(F)F)C4)nc21] |
| 206 | 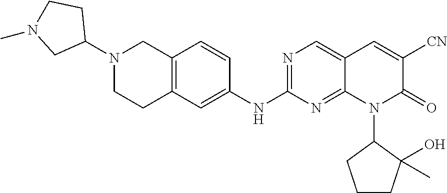 [SMILES: CN1CCC(N2CCc3cc(Nc4ncc5cc(C#N)c(=O)n(C6CCCC6(C)O)c5n4)ccc3C2)C1] |
| 207 | 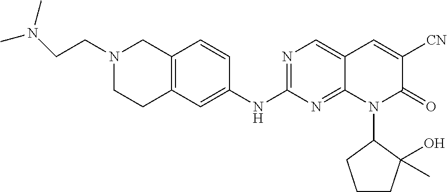 [SMILES: CN(C)CCN1CCc2cc(Nc3ncc4cc(C#N)c(=O)n(C5CCCC5(C)O)c4n3)ccc2C1] |
| 208 | 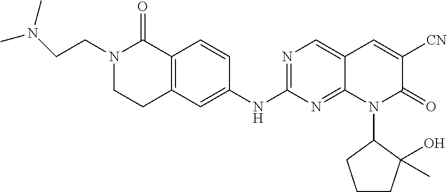 [SMILES: CN(C)CCN1CCc2cc(Nc3ncc4cc(C#N)c(=O)n(C5CCCC5(C)O)c4n3)ccc2C1=O] |
| 209 | 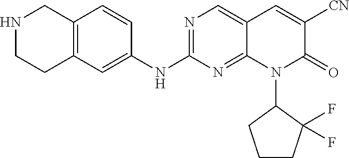 [SMILES: N#Cc1cc2cnc(Nc3ccc4c(c3)CCNC4)nc2n(C2CCCC2(F)F)c1=O] |
| 210 | 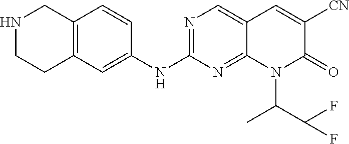 [SMILES: CC(C(F)F)n1c(=O)c(C#N)cc2cnc(Nc3ccc4c(c3)CCNC4)nc21] |
| 211 | 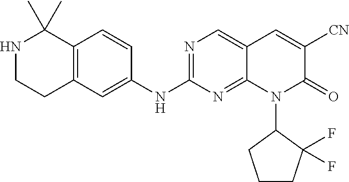 [SMILES: CC1(C)NCCc2cc(Nc3ncc4cc(C#N)c(=O)n(C5CCCC5(F)F)c4n3)ccc21] |
| 212 | 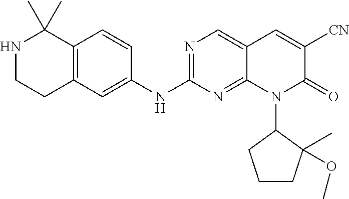 [SMILES: COC1(C)CCCC1n1c(=O)c(C#N)cc2cnc(Nc3ccc4c(c3)CCNC4(C)C)nc21] |
| 213 | 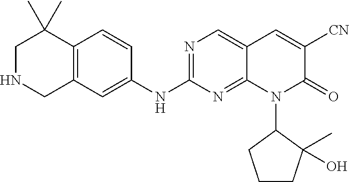 [SMILES: CC1(C)CNCc2cc(Nc3ncc4cc(C#N)c(=O)n(C5CCCC5(C)O)c4n3)ccc21] |
| 214 | 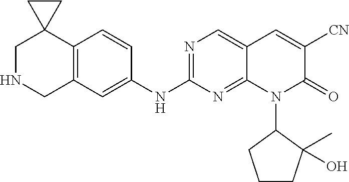 [SMILES: CC1(O)CCCC1n1c(=O)c(C#N)cc2cnc(Nc3ccc4c(c3)CNCC43CC3)nc21] |
| 215 | 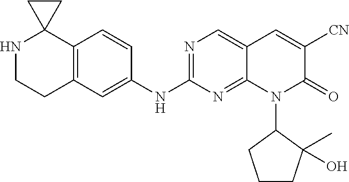 [SMILES: CC1(O)CCCC1n1c(=O)c(C#N)cc2cnc(Nc3ccc4c(c3)CCNC43CC3)nc21] |
| 216 | 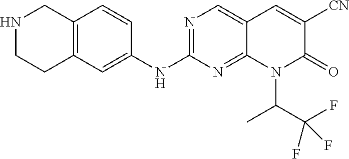 [SMILES: CC(n1c(=O)c(C#N)cc2cnc(Nc3ccc4c(c3)CCNC4)nc21)C(F)(F)F] |
| 217 | 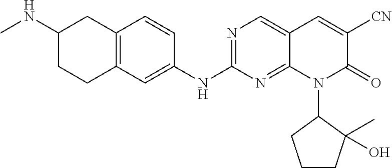 [SMILES: CNC1CCc2cc(Nc3ncc4cc(C#N)c(=O)n(C5CCCC5(C)O)c4n3)ccc2C1] |
| 218 | 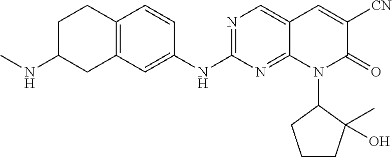 [SMILES: CNC1CCc2ccc(Nc3ncc4cc(C#N)c(=O)n(C5CCCC5(C)O)c4n3)cc2C1] |
| 219 | 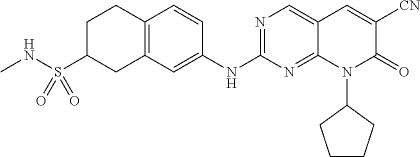 [SMILES: CNS(=O)(=O)C1CCc2ccc(Nc3ncc4cc(C#N)c(=O)n(C5CCCC5)c4n3)cc2C1] |
| 220 | 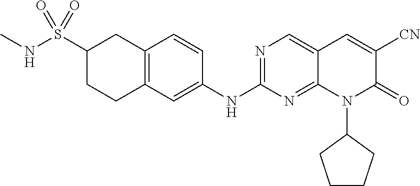 [SMILES: CNS(=O)(=O)C1CCc2cc(Nc3ncc4cc(C#N)c(=O)n(C5CCCC5)c4n3)ccc2C1] |
| 221 | 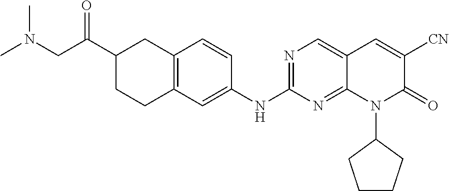 [SMILES: CN(C)CC(=O)C1CCc2cc(Nc3ncc4cc(C#N)c(=O)n(C5CCCC5)c4n3)ccc2C1] |
| 222 | 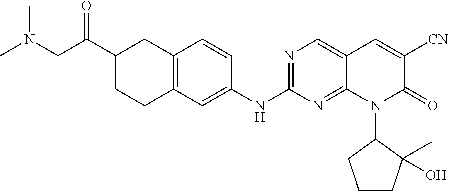 [SMILES: CN(C)CC(=O)C1CCc2cc(Nc3ncc4cc(C#N)c(=O)n(C5CCCC5(C)O)c4n3)ccc2C1] |
| 223 | 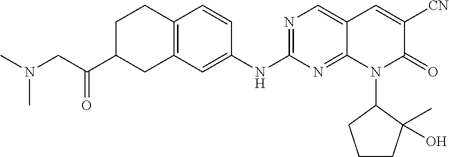 [SMILES: CN(C)CC(=O)C1CCc2ccc(Nc3ncc4cc(C#N)c(=O)n(C5CCCC5(C)O)c4n3)cc2C1] |
| 224 | 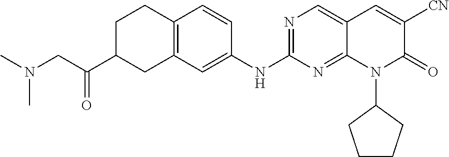 [SMILES: CN(C)CC(=O)C1CCc2ccc(Nc3ncc4cc(C#N)c(=O)n(C5CCCC5)c4n3)cc2C1] |
| 225 | 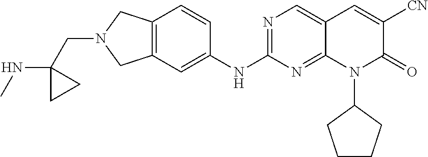 [SMILES: CNC1(CN2Cc3ccc(Nc4ncc5cc(C#N)c(=O)n(C6CCCC6)c5n4)cc3C2)CC1] |
| 226 | 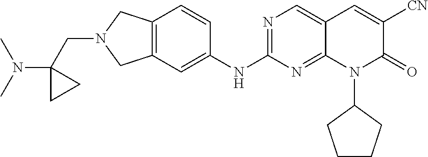 [SMILES: CN(C)C1(CN2Cc3ccc(Nc4ncc5cc(C#N)c(=O)n(C6CCCC6)c5n4)cc3C2)CC1] |
| 227 | 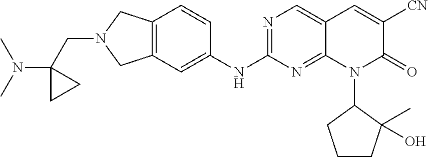 [SMILES: CN(C)C1(CN2Cc3ccc(Nc4ncc5cc(C#N)c(=O)n(C6CCCC6(C)O)c5n4)cc3C2)CC1] |
| 228 | 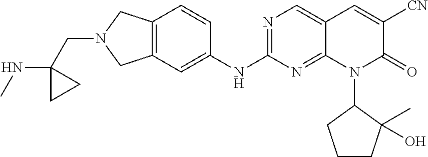 [SMILES: CNC1(CN2Cc3ccc(Nc4ncc5cc(C#N)c(=O)n(C6CCCC6(C)O)c5n4)cc3C2)CC1] |
| 229 | 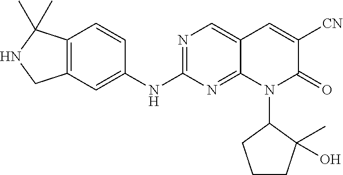 [SMILES: CC1(C)NCc2cc(Nc3ncc4cc(C#N)c(=O)n(C5CCCC5(C)O)c4n3)ccc21] |
| 230 | 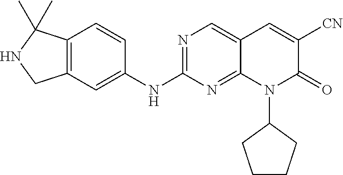 [SMILES: CC1(C)NCc2cc(Nc3ncc4cc(C#N)c(=O)n(C5CCCC5)c4n3)ccc21] |
| 231 | 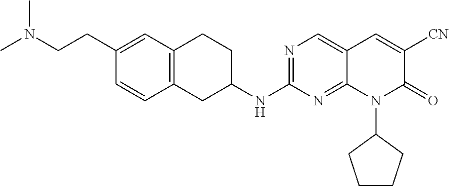 [SMILES: CN(C)CCc1ccc2c(c1)CCC(Nc1ncc3cc(C#N)c(=O)n(C4CCCC4)c3n1)C2] |
| 232 | 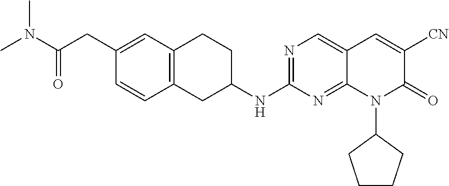 [SMILES: CN(C)C(=O)Cc1ccc2c(c1)CCC(Nc1ncc3cc(C#N)c(=O)n(C4CCCC4)c3n1)C2] |
| 233 | 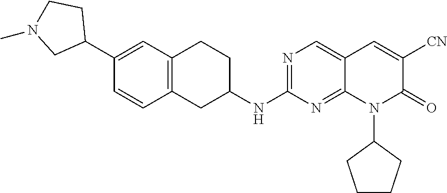 [SMILES: CN1CCC(c2ccc3c(c2)CCC(Nc2ncc4cc(C#N)c(=O)n(C5CCCC5)c4n2)C3)C1] |
| 234 | 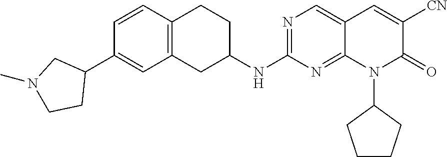 [SMILES: CN1CCC(c2ccc3c(c2)CC(Nc2ncc4cc(C#N)c(=O)n(C5CCCC5)c4n2)CC3)C1] |
| 235 | 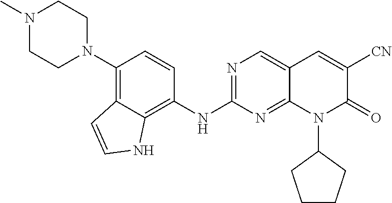 [SMILES: CN1CCN(c2ccc(Nc3ncc4cc(C#N)c(=O)n(C5CCCC5)c4n3)c3[nH]ccc23)CC1] |
| 236 | 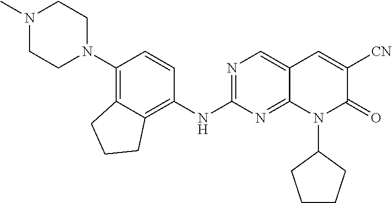 [SMILES: CN1CCN(c2ccc(Nc3ncc4cc(C#N)c(=O)n(C5CCCC5)c4n3)c3c2CCC3)CC1] |
| 237 | 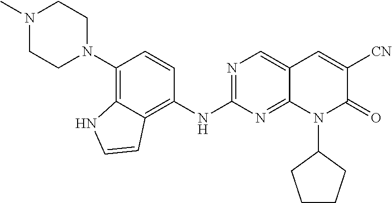 [SMILES: CN1CCN(c2ccc(Nc3ncc4cc(C#N)c(=O)n(C5CCCC5)c4n3)c3cc[nH]c23)CC1] |
| 238 | 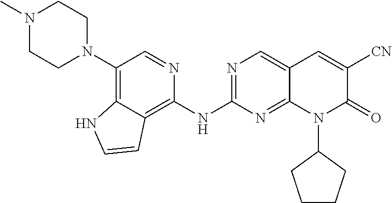 [SMILES: CN1CCN(c2cnc(Nc3ncc4cc(C#N)c(=O)n(C5CCCC5)c4n3)c3cc[nH]c23)CC1] |
| 239 | 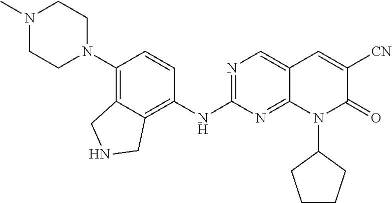 [SMILES: CN1CCN(c2ccc(Nc3ncc4cc(C#N)c(=O)n(C5CCCC5)c4n3)c3c2CNC3)CC1] |
| 240 | 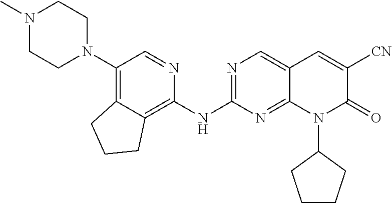 [SMILES: CN1CCN(c2cnc(Nc3ncc4cc(C#N)c(=O)n(C5CCCC5)c4n3)c3c2CCC3)CC1] |
| 241 | 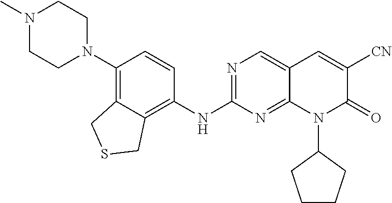 [SMILES: CN1CCN(c2ccc(Nc3ncc4cc(C#N)c(=O)n(C5CCCC5)c4n3)c3c2CSC3)CC1] |
| 242 | 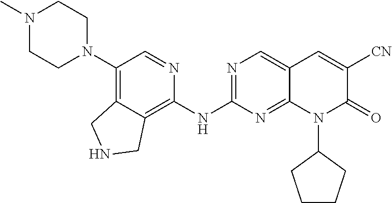 [SMILES: CN1CCN(c2cnc(Nc3ncc4cc(C#N)c(=O)n(C5CCCC5)c4n3)c3c2CNC3)CC1] |
| 243 | 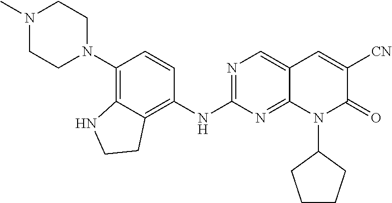 [SMILES: CN1CCN(c2ccc(Nc3ncc4cc(C#N)c(=O)n(C5CCCC5)c4n3)c3c2NCC3)CC1] |
| 244 | 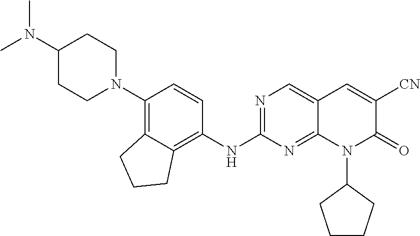 [SMILES: CN(C)C1CCN(c2ccc(Nc3ncc4cc(C#N)c(=O)n(C5CCCC5)c4n3)c3c2CCC3)CC1] |
| 245 | 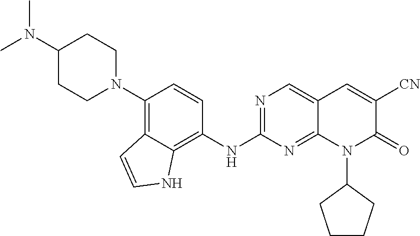 [SMILES: CN(C)C1CCN(c2ccc(Nc3ncc4cc(C#N)c(=O)n(C5CCCC5)c4n3)c3[nH]ccc23)CC1] |
| 246 | 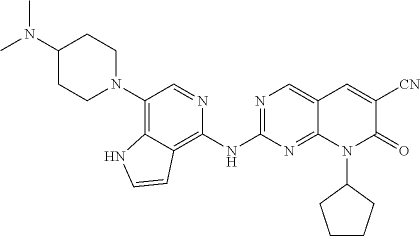 [SMILES: CN(C)C1CCN(c2cnc(Nc3ncc4cc(C#N)c(=O)n(C5CCCC5)c4n3)c3cc[nH]c23)CC1] |
| 247 | 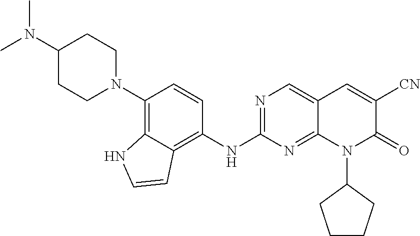 [SMILES: CN(C)C1CCN(c2ccc(Nc3ncc4cc(C#N)c(=O)n(C5CCCC5)c4n3)c3cc[nH]c23)CC1] |
| 248 | 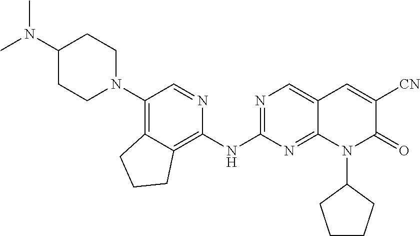 [SMILES: CN(C)C1CCN(c2cnc(Nc3ncc4cc(C#N)c(=O)n(C5CCCC5)c4n3)c3c2CCC3)CC1] |
| 249 | 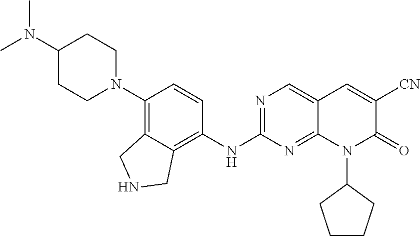 [SMILES: CN(C)C1CCN(c2ccc(Nc3ncc4cc(C#N)c(=O)n(C5CCCC5)c4n3)c3c2CNC3)CC1] |
| 250 | 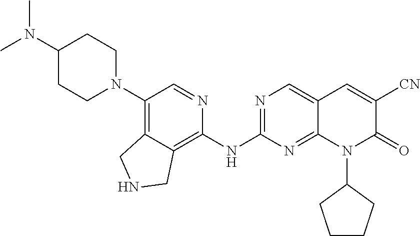 [SMILES: CN(C)C1CCN(c2cnc(Nc3ncc4cc(C#N)c(=O)n(C5CCCC5)c4n3)c3c2CNC3)CC1] |
| 251 | 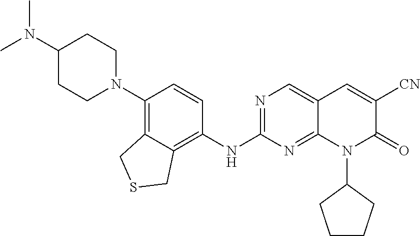 [SMILES: CN(C)C1CCN(c2ccc(Nc3ncc4cc(C#N)c(=O)n(C5CCCC5)c4n3)c3c2CSC3)CC1] |
| 252 | 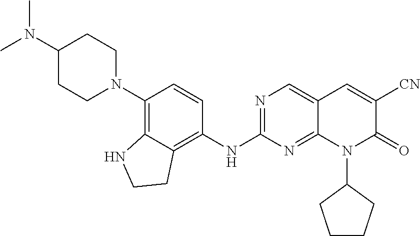 [SMILES: CN(C)C1CCN(c2ccc(Nc3ncc4cc(C#N)c(=O)n(C5CCCC5)c4n3)c3c2NCC3)CC1] |
| 253 | 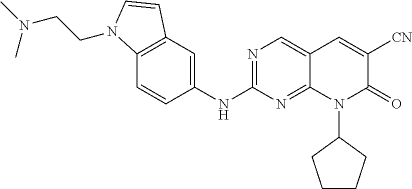 [SMILES: CN(C)CCn1ccc2cc(Nc3ncc4cc(C#N)c(=O)n(C5CCCC5)c4n3)ccc21] |
| 254 | 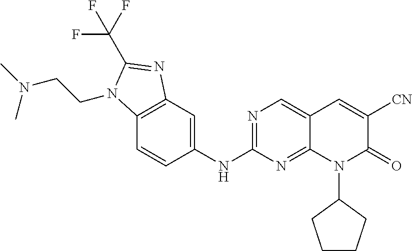 [SMILES: CN(C)CCn1c(C(F)(F)F)nc2cc(Nc3ncc4cc(C#N)c(=O)n(C5CCCC5)c4n3)ccc21] |
| 255 | 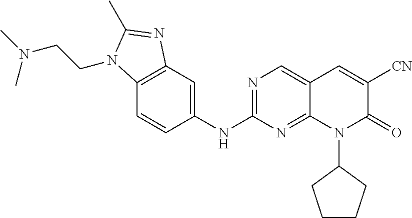 [SMILES: Cc1nc2cc(Nc3ncc4cc(C#N)c(=O)n(C5CCCC5)c4n3)ccc2n1CCN(C)C] |
| 256 | 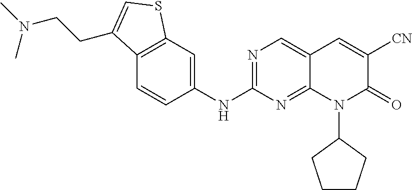 [SMILES: CN(C)CCc1csc2cc(Nc3ncc4cc(C#N)c(=O)n(C5CCCC5)c4n3)ccc12] |
| 257 | 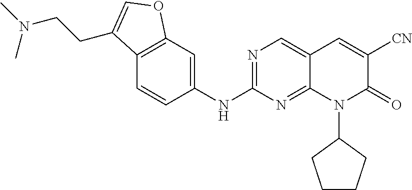 [SMILES: CN(C)CCc1coc2cc(Nc3ncc4cc(C#N)c(=O)n(C5CCCC5)c4n3)ccc12] |
| 258 | 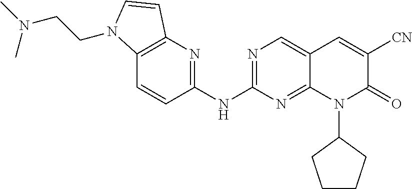 [SMILES: CN(C)CCn1ccc2nc(Nc3ncc4cc(C#N)c(=O)n(C5CCCC5)c4n3)ccc21] |
| 259 | 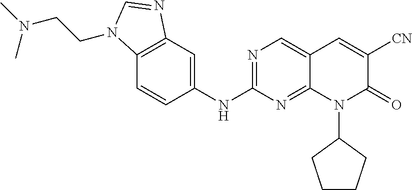 [SMILES: CN(C)CCn1cnc2cc(Nc3ncc4cc(C#N)c(=O)n(C5CCCC5)c4n3)ccc21] |
| 260 | 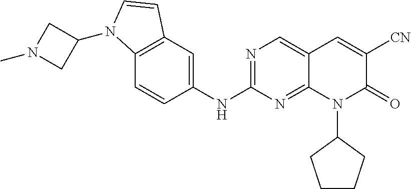 [SMILES: CN1CC(n2ccc3cc(Nc4ncc5cc(C#N)c(=O)n(C6CCCC6)c5n4)ccc32)C1] |
| 261 | 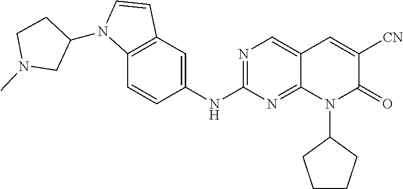 [SMILES: CN1CCC(n2ccc3cc(Nc4ncc5cc(C#N)c(=O)n(C6CCCC6)c5n4)ccc32)C1] |
| 262 | 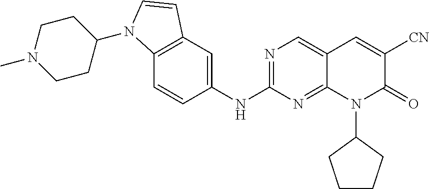 [SMILES: CN1CCC(n2ccc3cc(Nc4ncc5cc(C#N)c(=O)n(C6CCCC6)c5n4)ccc32)CC1] |
| 263 | 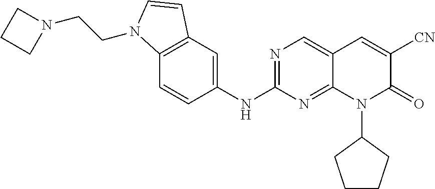 [SMILES: N#Cc1cc2cnc(Nc3ccc4c(ccn4CCN4CCC4)c3)nc2n(C2CCCC2)c1=O] |
| 264 | 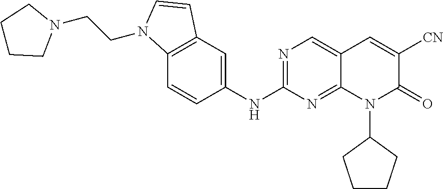 [SMILES: N#Cc1cc2cnc(Nc3ccc4c(ccn4CCN4CCCC4)c3)nc2n(C2CCCC2)c1=O] |
| 265 | 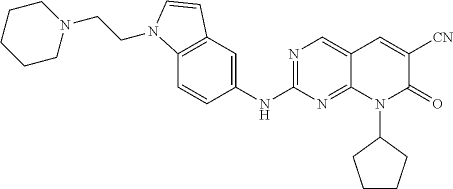 [SMILES: N#Cc1cc2cnc(Nc3ccc4c(ccn4CCN4CCCCC4)c3)nc2n(C2CCCC2)c1=O] |
| 266 | 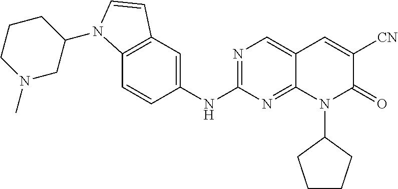 [SMILES: CN1CCCC(n2ccc3cc(Nc4ncc5cc(C#N)c(=O)n(C6CCCC6)c5n4)ccc32)C1] |
| 267 | 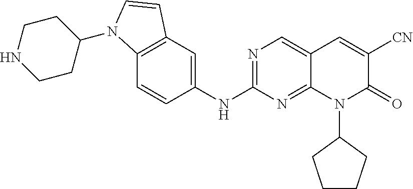 [SMILES: N#Cc1cc2cnc(Nc3ccc4c(ccn4C4CCNCC4)c3)nc2n(C2CCCC2)c1=O] |
| 268 | 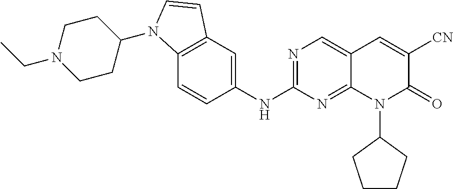 [SMILES: CCN1CCC(n2ccc3cc(Nc4ncc5cc(C#N)c(=O)n(C6CCCC6)c5n4)ccc32)CC1] |
| 269 | 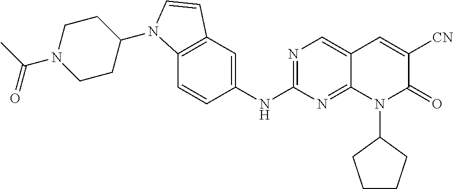 [SMILES: CC(=O)N1CCC(n2ccc3cc(Nc4ncc5cc(C#N)c(=O)n(C6CCCC6)c5n4)ccc32)CC1] |
| 270 | 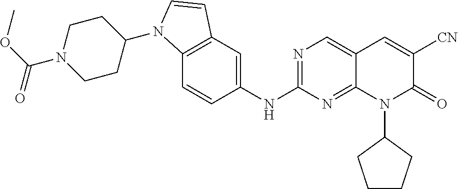 [SMILES: COC(=O)N1CCC(n2ccc3cc(Nc4ncc5cc(C#N)c(=O)n(C6CCCC6)c5n4)ccc32)CC1] |
| 271 | 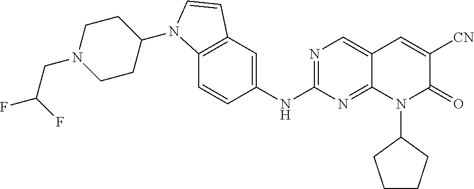 [SMILES: N#Cc1cc2cnc(Nc3ccc4c(ccn4C4CCN(CC(F)F)CC4)c3)nc2n(C2CCCC2)c1=O] |
| 272 | 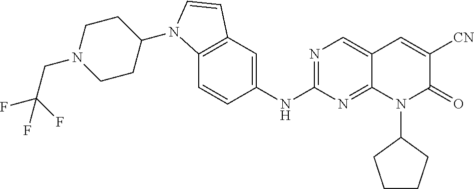 [SMILES: N#Cc1cc2cnc(Nc3ccc4c(ccn4C4CCN(CC(F)(F)F)CC4)c3)nc2n(C2CCCC2)c1=O] |
| 273 | 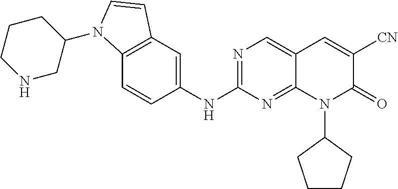 [SMILES: N#Cc1cc2cnc(Nc3ccc4c(ccn4C4CCCNC4)c3)nc2n(C2CCCC2)c1=O] |
| 274 | 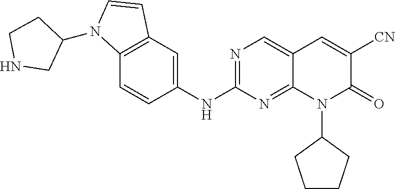 [SMILES: N#Cc1cc2cnc(Nc3ccc4c(ccn4C4CCNC4)c3)nc2n(C2CCCC2)c1=O] |
| 275 | 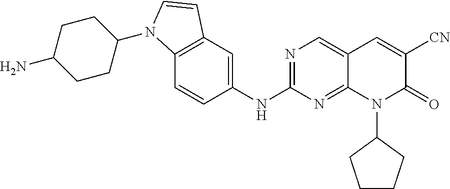 [SMILES: N#Cc1cc2cnc(Nc3ccc4c(ccn4C4CCC(N)CC4)c3)nc2n(C2CCCC2)c1=O] |
| 276 | 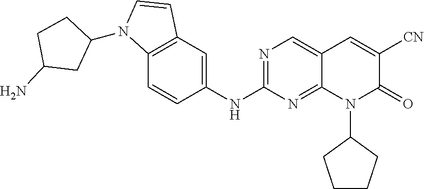 [SMILES: N#Cc1cc2cnc(Nc3ccc4c(ccn4C4CCC(N)C4)c3)nc2n(C2CCCC2)c1=O] |
| 277 | 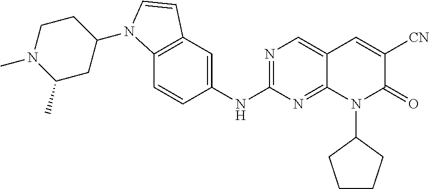 [SMILES: C[C@H]1CC(n2ccc3cc(Nc4ncc5cc(C#N)c(=O)n(C6CCCC6)c5n4)ccc32)CCN1C] |
| 278 | 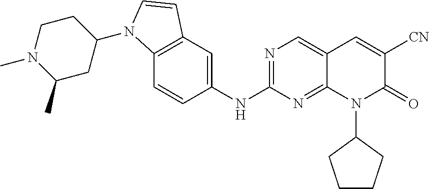 [SMILES: C[C@@H]1CC(n2ccc3cc(Nc4ncc5cc(C#N)c(=O)n(C6CCCC6)c5n4)ccc32)CCN1C] |
| 279 | 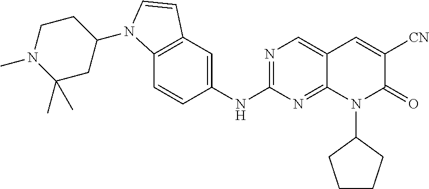 [SMILES: CN1CCC(n2ccc3cc(Nc4ncc5cc(C#N)c(=O)n(C6CCCC6)c5n4)ccc32)CC1(C)C] |
| 280 | 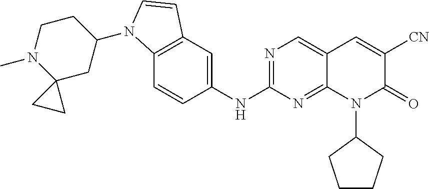 [SMILES: CN1CCC(n2ccc3cc(Nc4ncc5cc(C#N)c(=O)n(C6CCCC6)c5n4)ccc32)CC12CC2] |
| 281 | 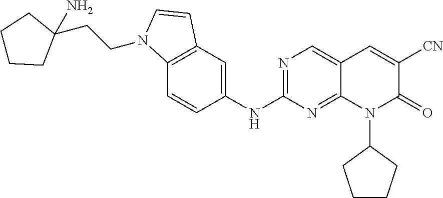 [SMILES: N#Cc1cc2cnc(Nc3ccc4c(ccn4CCC4(N)CCCC4)c3)nc2n(C2CCCC2)c1=O] |
| 282 | 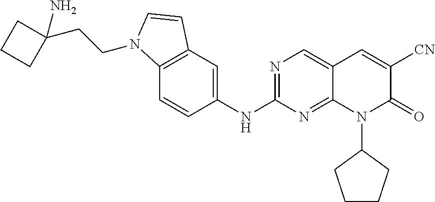 [SMILES: N#Cc1cc2cnc(Nc3ccc4c(ccn4CCC4(N)CCC4)c3)nc2n(C2CCCC2)c1=O] |
| 283 | 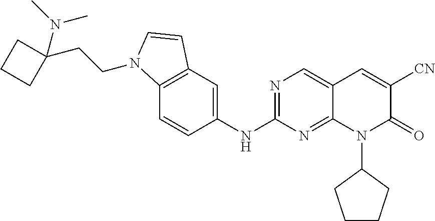 [SMILES: CN(C)C1(CCn2ccc3cc(Nc4ncc5cc(C#N)c(=O)n(C6CCCC6)c5n4)ccc32)CCC1] |
| 284 | 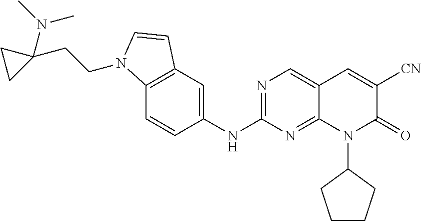 [SMILES: CN(C)C1(CCn2ccc3cc(Nc4ncc5cc(C#N)c(=O)n(C6CCCC6)c5n4)ccc32)CC1] |
| 285 | 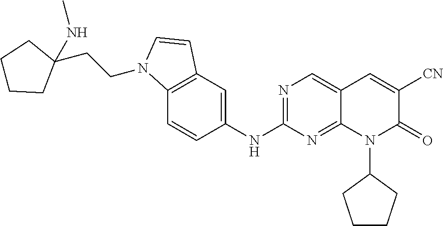 [SMILES: CNC1(CCn2ccc3cc(Nc4ncc5cc(C#N)c(=O)n(C6CCCC6)c5n4)ccc32)CCCC1] |
| 286 | 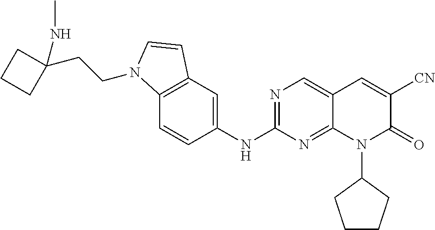 [SMILES: CNC1(CCn2ccc3cc(Nc4ncc5cc(C#N)c(=O)n(C6CCCC6)c5n4)ccc32)CCC1] |
| 287 | 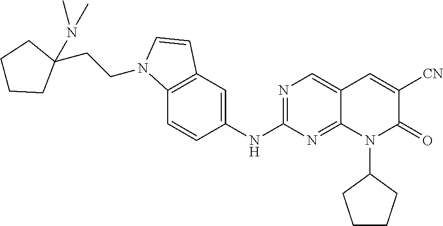 [SMILES: CN(C)C1(CCn2ccc3cc(Nc4ncc5cc(C#N)c(=O)n(C6CCCC6)c5n4)ccc32)CCCC1] |
| 288 | 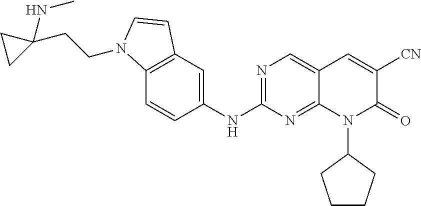 [SMILES: CNC1(CCn2ccc3cc(Nc4ncc5cc(C#N)c(=O)n(C6CCCC6)c5n4)ccc32)CC1] |
| 289 | 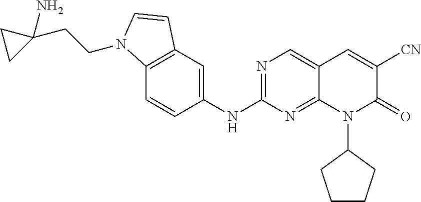 [SMILES: N#Cc1cc2cnc(Nc3ccc4c(ccn4CCC4(N)CC4)c3)nc2n(C2CCCC2)c1=O] |
| 290 | 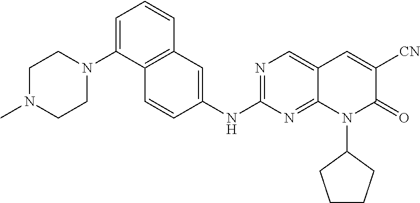 [SMILES: CN1CCN(c2cccc3cc(Nc4ncc5cc(C#N)c(=O)n(C6CCCC6)c5n4)ccc23)CC1] |
| 291 | 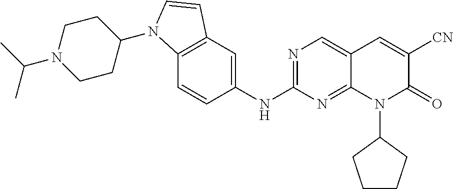 [SMILES: CC(C)N1CCC(n2ccc3cc(Nc4ncc5cc(C#N)c(=O)n(C6CCCC6)c5n4)ccc32)CC1] |
| 292 | 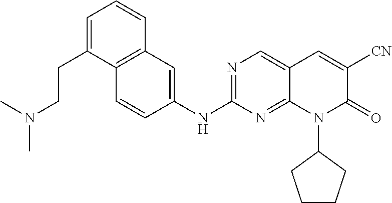 [SMILES: CN(C)CCc1cccc2cc(Nc3ncc4cc(C#N)c(=O)n(C5CCCC5)c4n3)ccc12] |
| 293 | 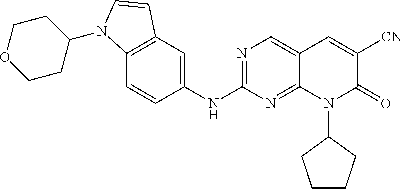 [SMILES: N#Cc1cc2cnc(Nc3ccc4c(ccn4C4CCOCC4)c3)nc2n(C2CCCC2)c1=O] |
| 294 | 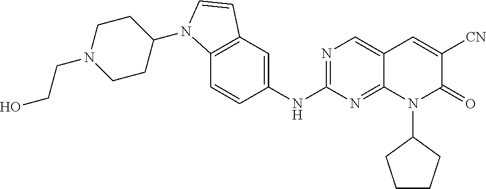 [SMILES: N#Cc1cc2cnc(Nc3ccc4c(ccn4C4CCN(CCO)CC4)c3)nc2n(C2CCCC2)c1=O] |
| 295 | 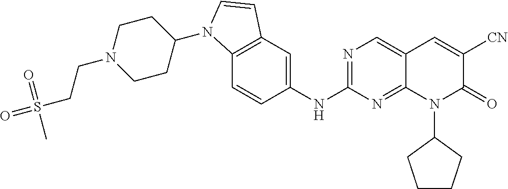 [SMILES: CS(=O)(=O)CCN1CCC(n2ccc3cc(Nc4ncc5cc(C#N)c(=O)n(C6CCCC6)c5n4)ccc32)CC1] |
| 296 | 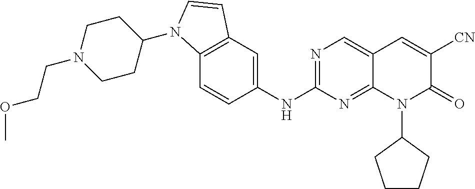 [SMILES: COCCN1CCC(n2ccc3cc(Nc4ncc5cc(C#N)c(=O)n(C6CCCC6)c5n4)ccc32)CC1] |
| 297 | 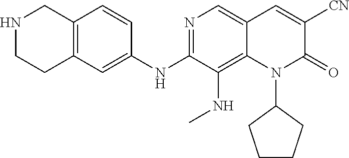 [SMILES: CNc1c(Nc2ccc3c(c2)CCNC3)ncc2cc(C#N)c(=O)n(C3CCCC3)c12] |
| 298 | 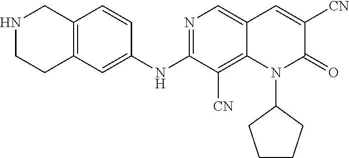 [SMILES: N#Cc1cc2cnc(Nc3ccc4c(c3)CCNC4)c(C#N)c2n(C2CCCC2)c1=O] |
| 299 | 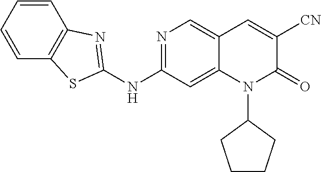 [SMILES: N#Cc1cc2cnc(Nc3nc4ccccc4s3)cc2n(C2CCCC2)c1=O] |
| 300 | 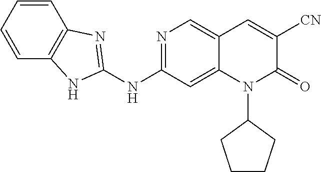 [SMILES: N#Cc1cc2cnc(Nc3nc4ccccc4[nH]3)cc2n(C2CCCC2)c1=O] |
| 301 | 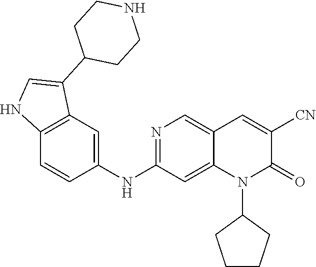 [SMILES: N#Cc1cc2cnc(Nc3ccc4[nH]cc(C5CCNCC5)c4c3)cc2n(C2CCCC2)c1=O] |
| 302 | 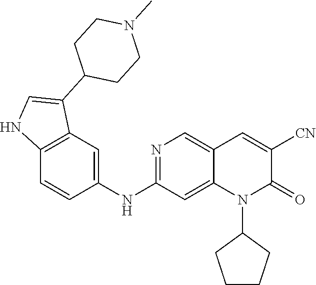 [SMILES: CN1CCC(c2c[nH]c3ccc(Nc4cc5c(cn4)cc(C#N)c(=O)n5C4CCCC4)cc23)CC1] |
| 303 | 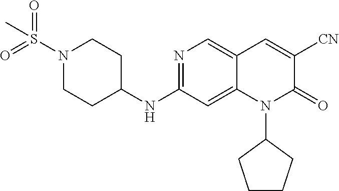 [SMILES: CS(=O)(=O)N1CCC(Nc2cc3c(cn2)cc(C#N)c(=O)n3C2CCCC2)CC1] |
| 304 | 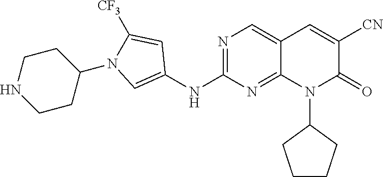 [SMILES: N#Cc1cc2cnc(Nc3cc(C(F)(F)F)n(C4CCNCC4)c3)nc2n(C2CCCC2)c1=O] |
| 305 | 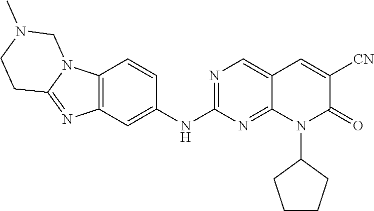 [SMILES: CN1CCc2nc3cc(Nc4ncc5cc(C#N)c(=O)n(C6CCCC6)c5n4)ccc3n2C1] |
| 306 | 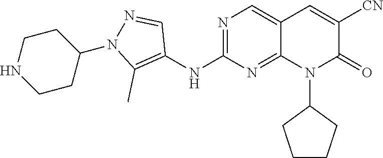 [SMILES: Cc1c(Nc2ncc3cc(C#N)c(=O)n(C4CCCC4)c3n2)cnn1C1CCNCC1] |
| 307 | 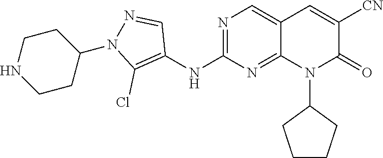 [SMILES: N#Cc1cc2cnc(Nc3cnn(C4CCNCC4)c3Cl)nc2n(C2CCCC2)c1=O] |
| 308 | 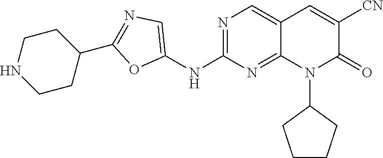 [SMILES: N#Cc1cc2cnc(Nc3cnc(C4CCNCC4)o3)nc2n(C2CCCC2)c1=O] |
| 309 | 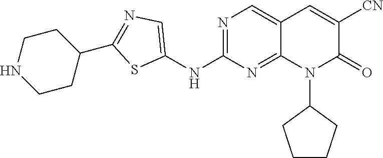 [SMILES: N#Cc1cc2cnc(Nc3cnc(C4CCNCC4)s3)nc2n(C2CCCC2)c1=O] |
| 310 | 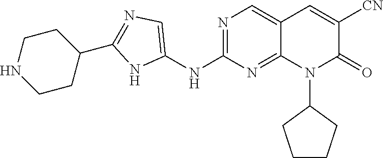 [SMILES: N#Cc1cc2cnc(Nc3cnc(C4CCNCC4)[nH]3)nc2n(C2CCCC2)c1=O] |
| 311 | 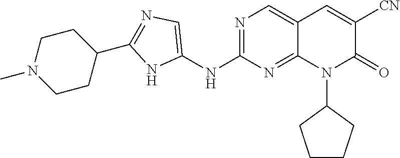 [SMILES: CN1CCC(c2ncc(Nc3ncc4cc(C#N)c(=O)n(C5CCCC5)c4n3)[nH]2)CC1] |
| 312 | 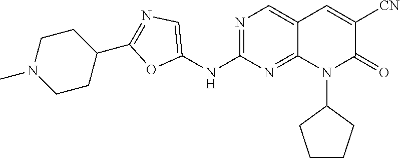 [SMILES: CN1CCC(c2ncc(Nc3ncc4cc(C#N)c(=O)n(C5CCCC5)c4n3)o2)CC1] |
| 313 | 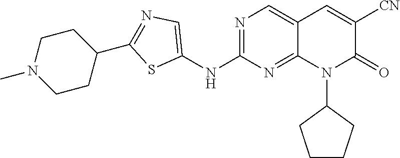 [SMILES: CN1CCC(c2ncc(Nc3ncc4cc(C#N)c(=O)n(C5CCCC5)c4n3)s2)CC1] |
| 314 | 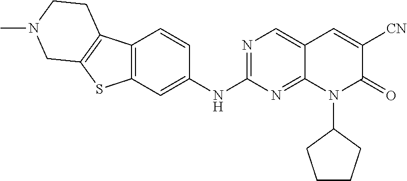 [SMILES: CN1CCc2c(sc3cc(Nc4ncc5cc(C#N)c(=O)n(C6CCCC6)c5n4)ccc23)C1] |
| 315 | 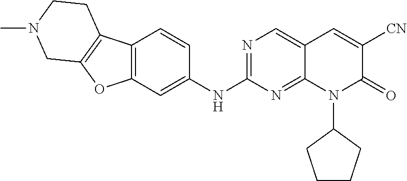 [SMILES: CN1CCc2c(oc3cc(Nc4ncc5cc(C#N)c(=O)n(C6CCCC6)c5n4)ccc23)C1] |
| 316 | 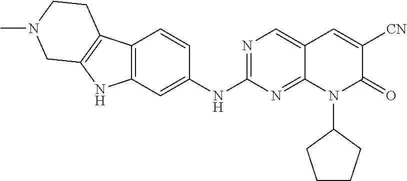 [SMILES: CN1CCc2c([nH]c3cc(Nc4ncc5cc(C#N)c(=O)n(C6CCCC6)c5n4)ccc23)C1] |
| 317 | 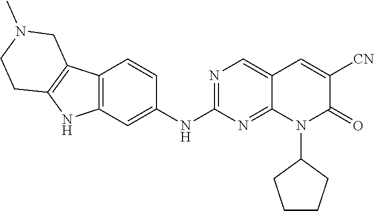 [SMILES: CN1CCc2[nH]c3cc(Nc4ncc5cc(C#N)c(=O)n(C6CCCC6)c5n4)ccc3c2C1] |
| 318 | 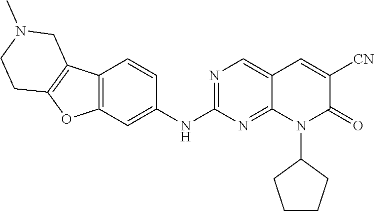 [SMILES: CN1CCc2oc3cc(Nc4ncc5cc(C#N)c(=O)n(C6CCCC6)c5n4)ccc3c2C1] |
| 319 | 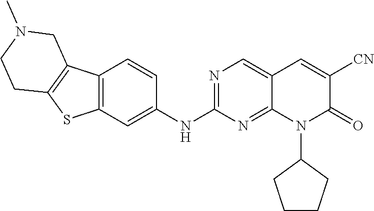 [SMILES: CN1CCc2sc3cc(Nc4ncc5cc(C#N)c(=O)n(C6CCCC6)c5n4)ccc3c2C1] |
| 320 | 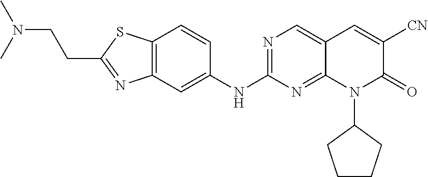 [SMILES: CN(C)CCc1nc2cc(Nc3ncc4cc(C#N)c(=O)n(C5CCCC5)c4n3)ccc2s1] |
| 321 | 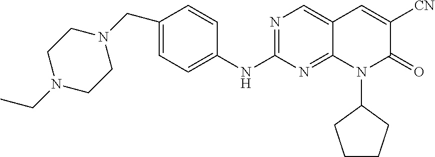 [SMILES: CCN1CCN(Cc2ccc(Nc3ncc4cc(C#N)c(=O)n(C5CCCC5)c4n3)cc2)CC1] |
| 322 | 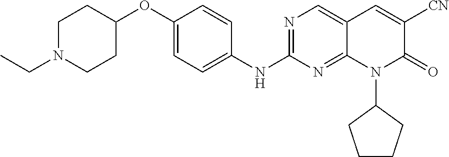 [SMILES: CCN1CCC(Oc2ccc(Nc3ncc4cc(C#N)c(=O)n(C5CCCC5)c4n3)cc2)CC1] |
| 323 | 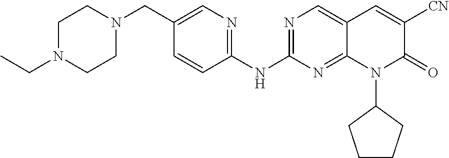 [SMILES: CCN1CCN(Cc2ccc(Nc3ncc4cc(C#N)c(=O)n(C5CCCC5)c4n3)nc2)CC1] |
| 324 | 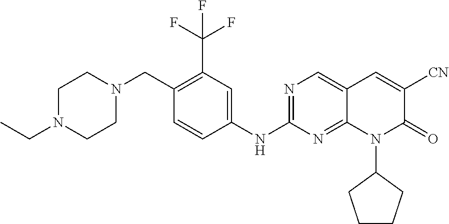 [SMILES: CCN1CCN(Cc2ccc(Nc3ncc4cc(C#N)c(=O)n(C5CCCC5)c4n3)cc2C(F)(F)F)CC1] |
| 325 | 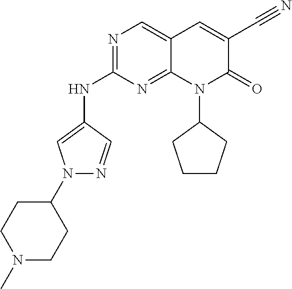 [SMILES: CN1CCC(n2cc(Nc3ncc4cc(C#N)c(=O)n(C5CCCC5)c4n3)cn2)CC1] |
| 326 | 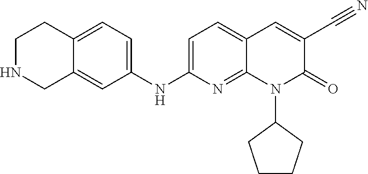 [SMILES: N#Cc1cc2ccc(Nc3ccc4c(c3)CNCC4)nc2n(C2CCCC2)c1=O] |
| 327 | 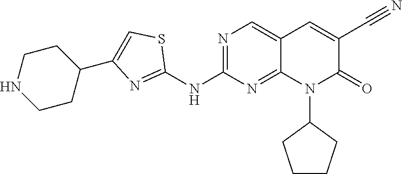 [SMILES: N#Cc1cc2cnc(Nc3nc(C4CCNCC4)cs3)nc2n(C2CCCC2)c1=O] |
| 328 | 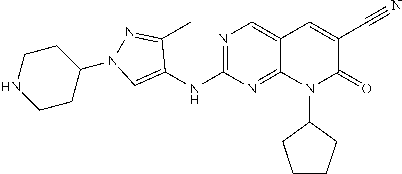 [SMILES: Cc1nn(C2CCNCC2)cc1Nc1ncc2cc(C#N)c(=O)n(C3CCCC3)c2n1] |
| 329 | 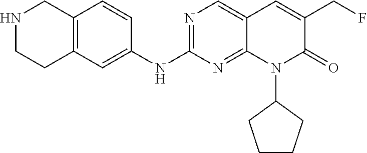 [SMILES: O=c1c(CF)cc2cnc(Nc3ccc4c(c3)CCNC4)nc2n1C1CCCC1] |
| 330 | 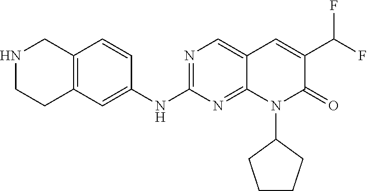 [SMILES: O=c1c(C(F)F)cc2cnc(Nc3ccc4c(c3)CCNC4)nc2n1C1CCCC1] |
| 331 | 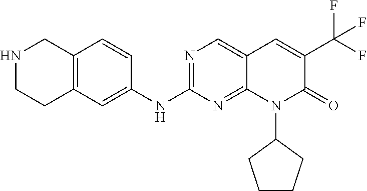 [SMILES: O=c1c(C(F)(F)F)cc2cnc(Nc3ccc4c(c3)CCNC4)nc2n1C1CCCC1] |
| 332 | 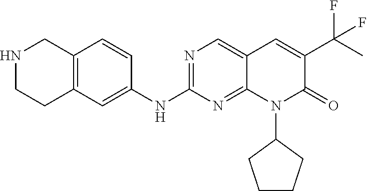 [SMILES: CC(F)(F)c1cc2cnc(Nc3ccc4c(c3)CCNC4)nc2n(C2CCCC2)c1=O] |
| 333 | 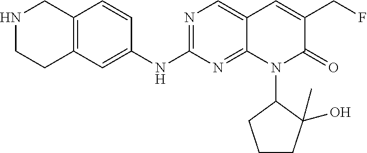 [SMILES: CC1(O)CCCC1n1c(=O)c(CF)cc2cnc(Nc3ccc4c(c3)CCNC4)nc21] |
| 334 | 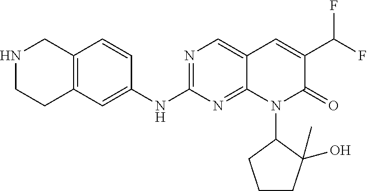 [SMILES: CC1(O)CCCC1n1c(=O)c(C(F)F)cc2cnc(Nc3ccc4c(c3)CCNC4)nc21] |
| 335 | 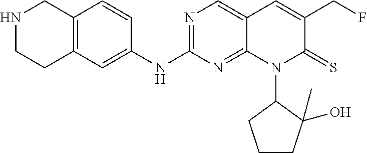 [SMILES: CC1(O)CCCC1n1c(=S)c(CF)cc2cnc(Nc3ccc4c(c3)CCNC4)nc21] |
| 336 | 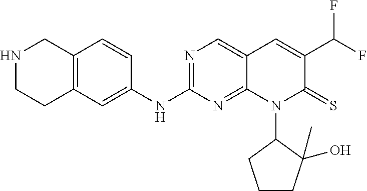 [SMILES: CC1(O)CCCC1n1c(=S)c(C(F)F)cc2cnc(Nc3ccc4c(c3)CCNC4)nc21] |
| 337 | 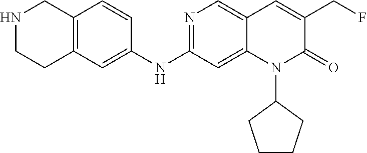 [SMILES: O=c1c(CF)cc2cnc(Nc3ccc4c(c3)CCNC4)cc2n1C1CCCC1] |
| 338 | 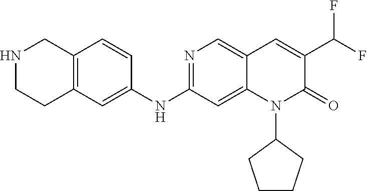 [SMILES: O=c1c(C(F)F)cc2cnc(Nc3ccc4c(c3)CCNC4)cc2n1C1CCCC1] |
| 339 | 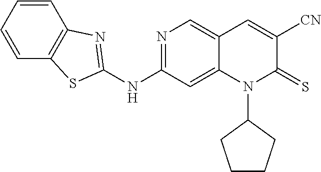 [SMILES: N#Cc1cc2cnc(Nc3nc4ccccc4s3)cc2n(C2CCCC2)c1=S] |
| 340 | 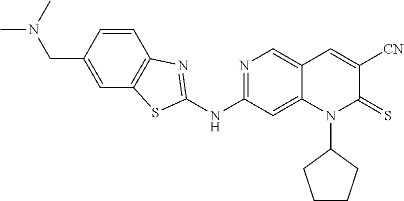 [SMILES: CN(C)Cc1ccc2nc(Nc3cc4c(cn3)cc(C#N)c(=S)n4C3CCCC3)sc2c1] |
| 341 | 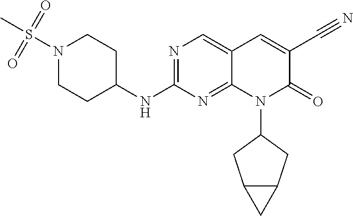 [SMILES: CS(=O)(=O)N1CCC(Nc2ncc3cc(C#N)c(=O)n(C4CC5CC5C4)c3n2)CC1] |
| 342 | 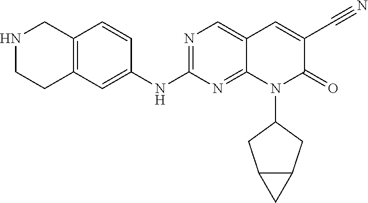 [SMILES: N#Cc1cc2cnc(Nc3ccc4c(c3)CCNC4)nc2n(C2CC3CC3C2)c1=O] |
| 343 | 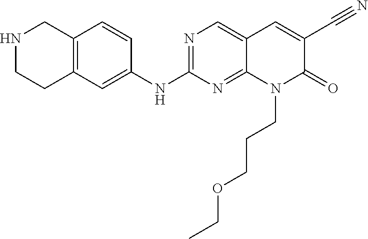 [SMILES: CCOCCCn1c(=O)c(C#N)cc2cnc(Nc3ccc4c(c3)CCNC4)nc21] |
| 344 | 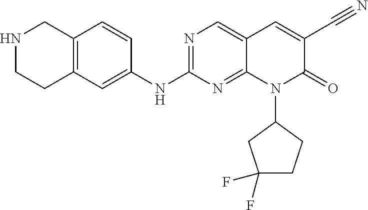 [SMILES: N#Cc1cc2cnc(Nc3ccc4c(c3)CCNC4)nc2n(C2CCC(F)(F)C2)c1=O] |
| 345 | 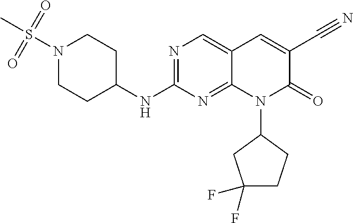 [SMILES: CS(=O)(=O)N1CCC(Nc2ncc3cc(C#N)c(=O)n(C4CCC(F)(F)C4)c3n2)CC1] |
| 346 | 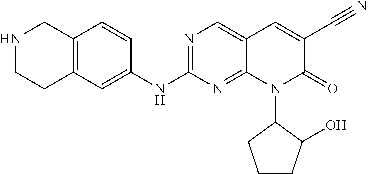 [SMILES: N#Cc1cc2cnc(Nc3ccc4c(c3)CCNC4)nc2n(C2CCCC2O)c1=O] |
| 347 | 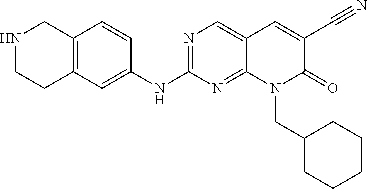 [SMILES: N#Cc1cc2cnc(Nc3ccc4c(c3)CCNC4)nc2n(CC2CCCCC2)c1=O] |
| 348 | 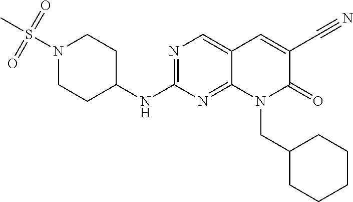 [SMILES: CS(=O)(=O)N1CCC(Nc2ncc3cc(C#N)c(=O)n(CC4CCCCC4)c3n2)CC1] |
| 349 | 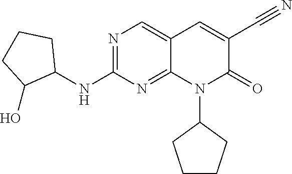 [SMILES: N#Cc1cc2cnc(NC3CCCC3O)nc2n(C2CCCC2)c1=O] |
| 350 | 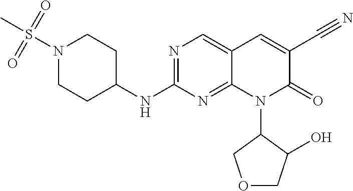 [SMILES: CS(=O)(=O)N1CCC(Nc2ncc3cc(C#N)c(=O)n(C4COCC4O)c3n2)CC1] |
| 351 | 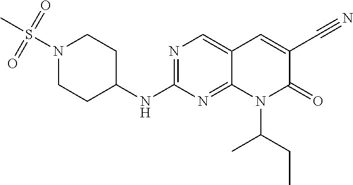 [SMILES: CCC(C)n1c(=O)c(C#N)cc2cnc(NC3CCN(S(C)(=O)=O)CC3)nc21] |
| 352 | 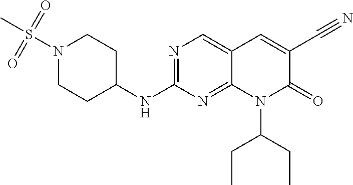 [SMILES: CCC(CC)n1c(=O)c(C#N)cc2cnc(NC3CCN(S(C)(=O)=O)CC3)nc21] |
| 353 | 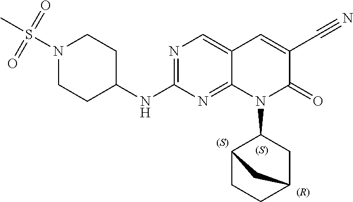 |
| 354 | 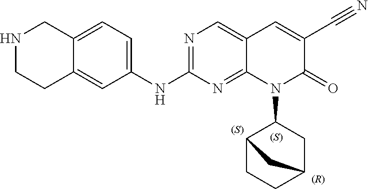 |
| 355 | 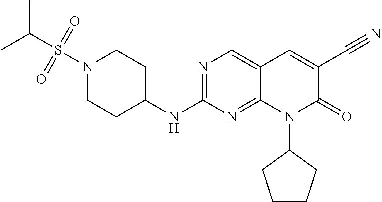 [SMILES: CC(C)S(=O)(=O)N1CCC(Nc2ncc3cc(C#N)c(=O)n(C4CCCC4)c3n2)CC1] |
| 356 | 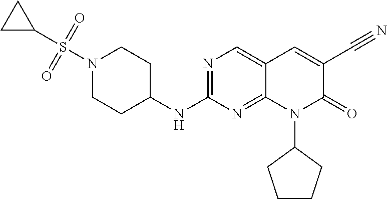 [SMILES: N#Cc1cc2cnc(NC3CCN(S(=O)(=O)C4CC4)CC3)nc2n(C2CCCC2)c1=O] |
| 357 | 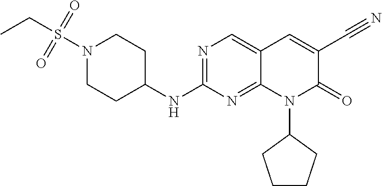 [SMILES: CCS(=O)(=O)N1CCC(Nc2ncc3cc(C#N)c(=O)n(C4CCCC4)c3n2)CC1] |
| 358 | 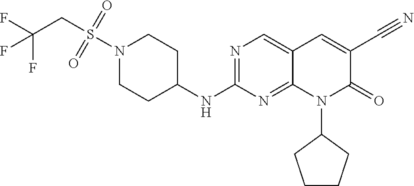 [SMILES: N#Cc1cc2cnc(NC3CCN(S(=O)(=O)CC(F)(F)F)CC3)nc2n(C2CCCC2)c1=O] |
| 359 | 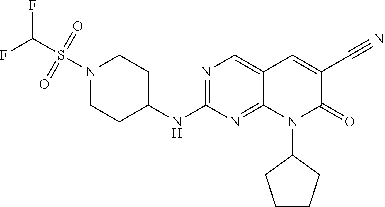 [SMILES: N#Cc1cc2cnc(NC3CCN(S(=O)(=O)C(F)F)CC3)nc2n(C2CCCC2)c1=O] |
| 360 | 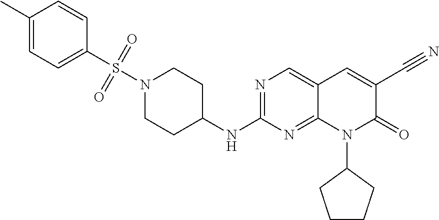 [SMILES: Cc1ccc(S(=O)(=O)N2CCC(Nc3ncc4cc(C#N)c(=O)n(C5CCCC5)c4n3)CC2)cc1] |
| 361 | 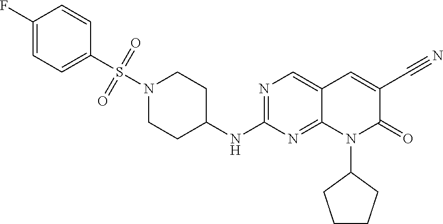 [SMILES: N#Cc1cc2cnc(NC3CCN(S(=O)(=O)c4ccc(F)cc4)CC3)nc2n(C2CCCC2)c1=O] |
| 362 | 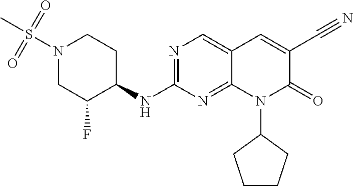 [SMILES: CS(=O)(=O)N1CC[C@@H](Nc2ncc3cc(C#N)c(=O)n(C4CCCC4)c3n2)[C@H](F)C1] |
| 363 | 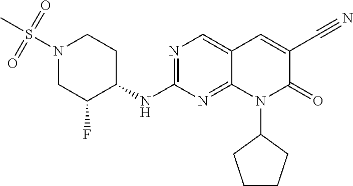 [SMILES: CS(=O)(=O)N1CC[C@H](Nc2ncc3cc(C#N)c(=O)n(C4CCCC4)c3n2)[C@H](F)C1] |
| 364 | 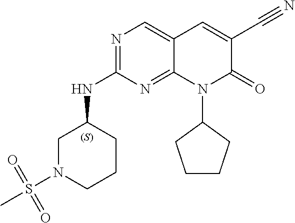 |
| 365 | 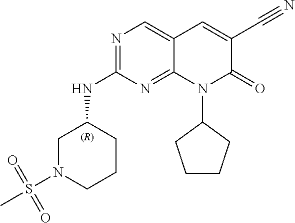 |
| 366 | 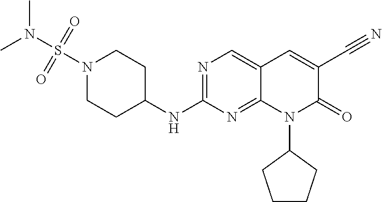 [SMILES: CN(C)S(=O)(=O)N1CCC(Nc2ncc3cc(C#N)c(=O)n(C4CCCC4)c3n2)CC1] |
| 367 | 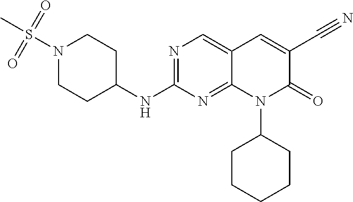 [SMILES: CS(=O)(=O)N1CCC(Nc2ncc3cc(C#N)c(=O)n(C4CCCCC4)c3n2)CC1] |
| 368 | 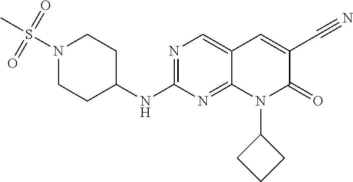 [SMILES: CS(=O)(=O)N1CCC(Nc2ncc3cc(C#N)c(=O)n(C4CCC4)c3n2)CC1] |
| 369 | 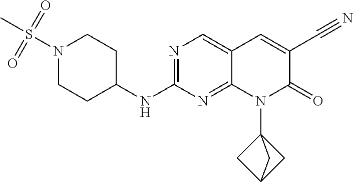 [SMILES: CS(=O)(=O)N1CCC(Nc2ncc3cc(C#N)c(=O)n(C45CC(C4)C5)c3n2)CC1] |
| 370 | 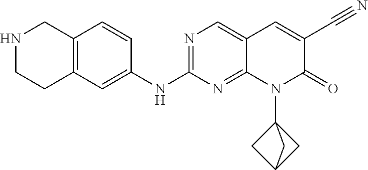 [SMILES: N#Cc1cc2cnc(Nc3ccc4c(c3)CCNC4)nc2n(C23CC(C2)C3)c1=O] |
| 371 | 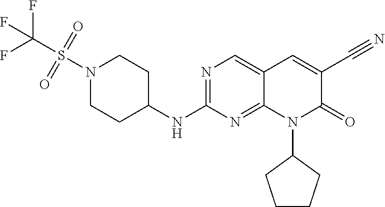 [SMILES: N#Cc1cc2cnc(NC3CCN(S(=O)(=O)C(F)(F)F)CC3)nc2n(C2CCCC2)c1=O] |
| 372 | 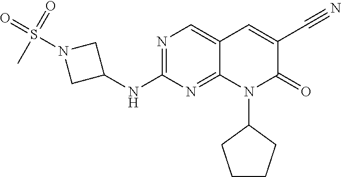 [SMILES: CS(=O)(=O)N1CC(Nc2ncc3cc(C#N)c(=O)n(C4CCCC4)c3n2)C1] |
| 373 | 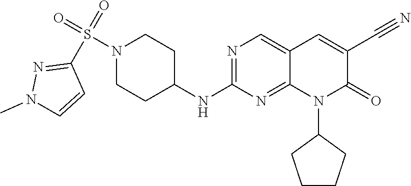 [SMILES: Cn1ccc(S(=O)(=O)N2CCC(Nc3ncc4cc(C#N)c(=O)n(C5CCCC5)c4n3)CC2)n1] |
| 374 | 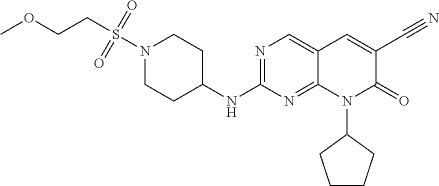 [SMILES: COCCS(=O)(=O)N1CCC(Nc2ncc3cc(C#N)c(=O)n(C4CCCC4)c3n2)CC1] |
| 375 | 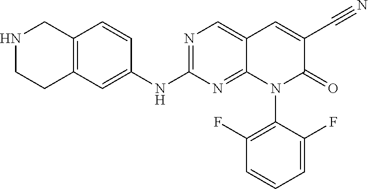 [SMILES: N#Cc1cc2cnc(Nc3ccc4c(c3)CCNC4)nc2n(-c2c(F)cccc2F)c1=O] |
| 376 | 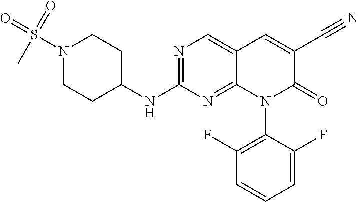 [SMILES: CS(=O)(=O)N1CCC(Nc2ncc3cc(C#N)c(=O)n(-c4c(F)cccc4F)c3n2)CC1] |
| 377 | 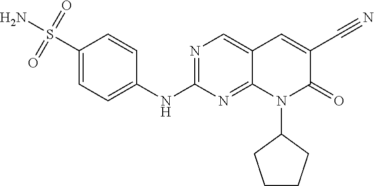 [SMILES: N#Cc1cc2cnc(Nc3ccc(S(N)(=O)=O)cc3)nc2n(C2CCCC2)c1=O] |
| 378 | 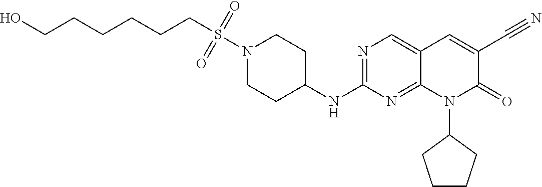 [SMILES: N#Cc1cc2cnc(NC3CCN(S(=O)(=O)CCCCCCO)CC3)nc2n(C2CCCC2)c1=O] |
| 379 | 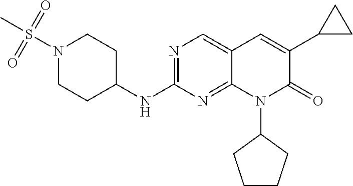 [SMILES: CS(=O)(=O)N1CCC(Nc2ncc3cc(C4CC4)c(=O)n(C4CCCC4)c3n2)CC1] |
| 380 | 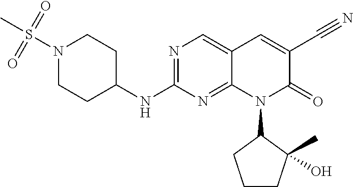 |
| 381 | 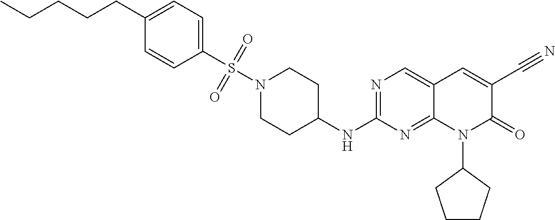 [SMILES: CCCCCc1ccc(S(=O)(=O)N2CCC(Nc3ncc4cc(C#N)c(=O)n(C5CCCC5)c4n3)CC2)cc1] |
| 382 | 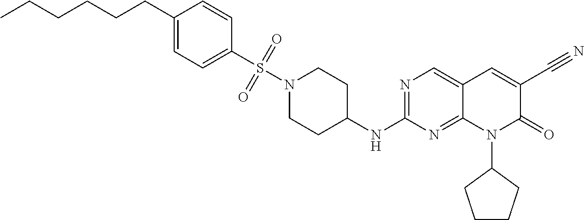 [SMILES: CCCCCCc1ccc(S(=O)(=O)N2CCC(Nc3ncc4cc(C#N)c(=O)n(C5CCCC5)c4n3)CC2)cc1] |
| 383 | 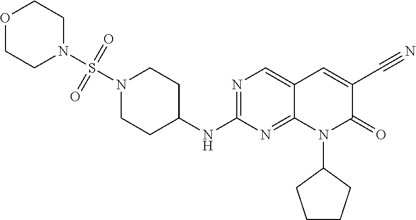 [SMILES: N#Cc1cc2cnc(NC3CCN(S(=O)(=O)N4CCOCC4)CC3)nc2n(C2CCCC2)c1=O] |
| 384 | 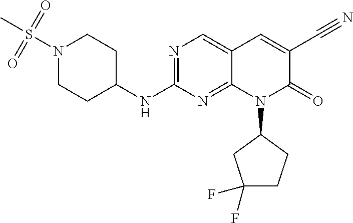 [SMILES: CS(=O)(=O)N1CCC(Nc2ncc3cc(C#N)c(=O)n([C@H]4CCC(F)(F)C4)c3n2)CC1] |
| 385 | 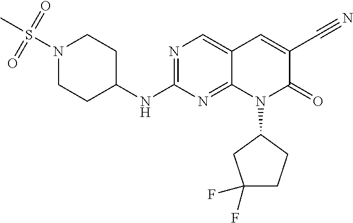 [SMILES: CS(=O)(=O)N1CCC(Nc2ncc3cc(C#N)c(=O)n([C@@H]4CCC(F)(F)C4)c3n2)CC1] |
| 386 | 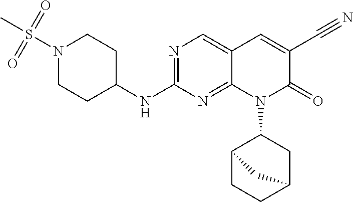 [SMILES: CS(=O)(=O)N1CCC(Nc2ncc3cc(C#N)c(=O)n([C@@H]4C[C@H]5CC[C@@H]4C5)c3n2)CC1] |
| 387 | 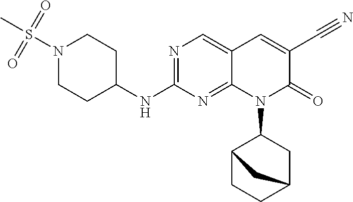 [SMILES: CS(=O)(=O)N1CCC(Nc2ncc3cc(C#N)c(=O)n([C@H]4C[C@@H]5CC[C@H]4C5)c3n2)CC1] |
| 388 | 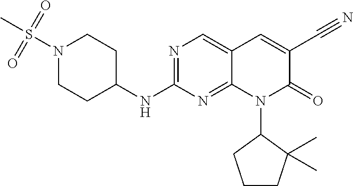 [SMILES: CC1(C)CCCC1n1c(=O)c(C#N)cc2cnc(NC3CCN(S(C)(=O)=O)CC3)nc21] |
| 389 | 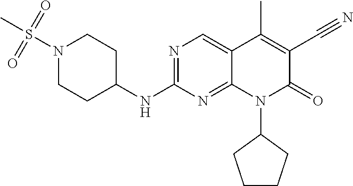 [SMILES: Cc1c(C#N)c(=O)n(C2CCCC2)c2nc(NC3CCN(S(C)(=O)=O)CC3)ncc12] |
| 390 | 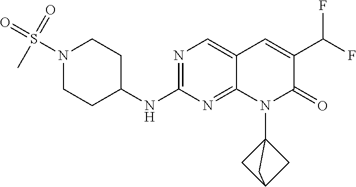 [SMILES: CS(=O)(=O)N1CCC(Nc2ncc3cc(C(F)F)c(=O)n(C45CC(C4)C5)c3n2)CC1] |
| 391 | 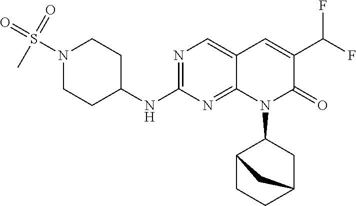 [SMILES: CS(=O)(=O)N1CCC(Nc2ncc3cc(C(F)F)c(=O)n([C@H]4C[C@@H]5CC[C@H]4C5)c3n2)CC1] |
| 392 | 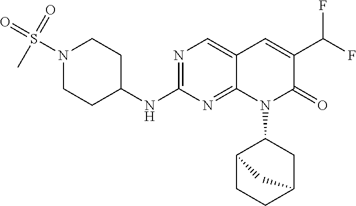 [SMILES: CS(=O)(=O)N1CCC(Nc2ncc3cc(C(F)F)c(=O)n([C@@H]4C[C@H]5CC[C@@H]4C5)c3n2)CC1] |
| 393 | 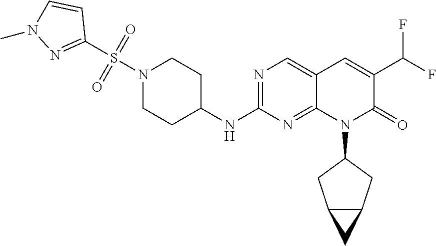 [SMILES: Cn1ccc(S(=O)(=O)N2CCC(Nc3ncc4cc(C(F)F)c(=O)n([C@H]5C[C@H]6C[C@H]6C5)c4n3)CC2)n1] |
| 394 | 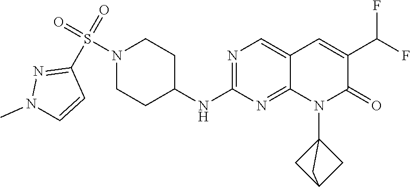 [SMILES: Cn1ccc(S(=O)(=O)N2CCC(Nc3ncc4cc(C(F)F)c(=O)n(C56CC(C5)C6)c4n3)CC2)n1] |
| 395 | 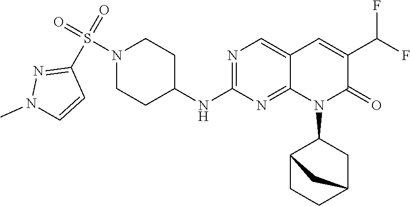 [SMILES: Cn1ccc(S(=O)(=O)N2CCC(Nc3ncc4cc(C(F)F)c(=O)n([C@H]5C[C@@H]6CC[C@H]5C6)c4n3)CC2)n1] |
| 396 | 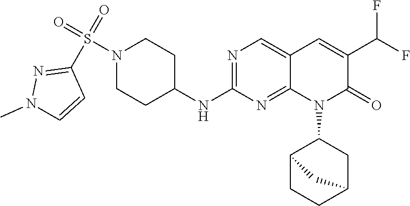 [SMILES: Cn1ccc(S(=O)(=O)N2CCC(Nc3ncc4cc(C(F)F)c(=O)n([C@@H]5C[C@H]6CC[C@@H]5C6)c4n3)CC2)n1] |
| 397 | 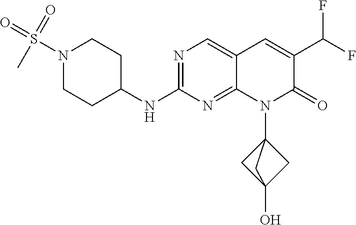 [SMILES: CS(=O)(=O)N1CCC(Nc2ncc3cc(C(F)F)c(=O)n(C45CC(O)(C4)C5)c3n2)CC1] |
| 398 | 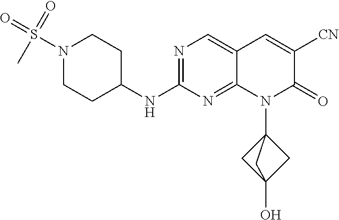 [SMILES: CS(=O)(=O)N1CCC(Nc2ncc3cc(C#N)c(=O)n(C45CC(O)(C4)C5)c3n2)CC1] |
| 399 | 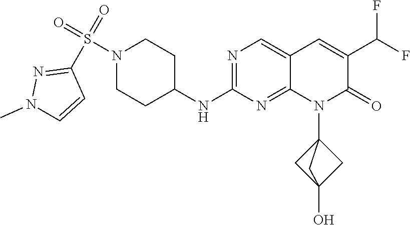 [SMILES: Cn1ccc(S(=O)(=O)N2CCC(Nc3ncc4cc(C(F)F)c(=O)n(C56CC(O)(C5)C6)c4n3)CC2)n1] |
| 400 | 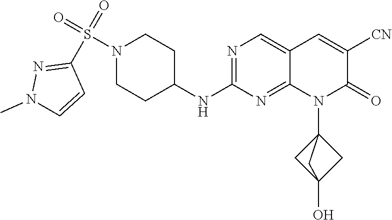 [SMILES: Cn1ccc(S(=O)(=O)N2CCC(Nc3ncc4cc(C#N)c(=O)n(C56CC(O)(C5)C6)c4n3)CC2)n1] |
| 401 | 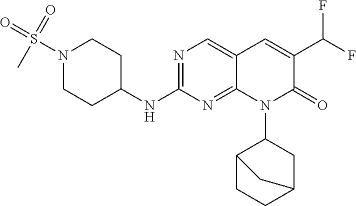 [SMILES: CS(=O)(=O)N1CCC(Nc2ncc3cc(C(F)F)c(=O)n(C4CC5CCC4C5)c3n2)CC1] |

In some embodiments, provided herein are compounds described in Table 1, or a tautomer thereof, or a salt of any of the foregoing, and uses thereof. In some embodiments, provided herein are compounds 1-195 and 197-340 of Table 1.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. The structure or name is intended to embrace all possible stereoisomers of a compound depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate

In some embodiments, compounds of Formula (I) or (II) may be synthesized according to Scheme 1. In some embodiments, compounds of Formula (K) may be synthesized according to Schemes 1 to 4.

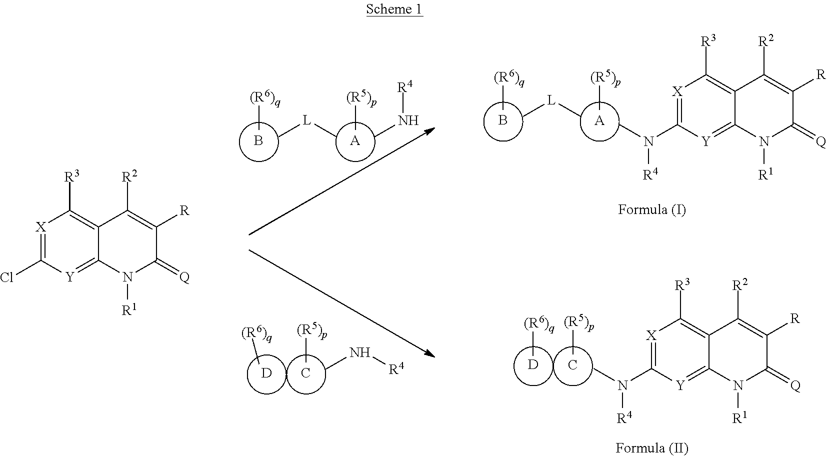

wherein X, Y, R, Q, A, B, C, D, L, R, R², R³, R⁴, R⁵, R⁶; p and q are as described for Formula (J), Formula (I), or Formula (II). In some embodiments, X, Y, R, Q, A, B, C, D, L, R¹, R², R³, R⁴, R⁵, R⁶; p and q are as described for Formula (K) or any related formulae, where applicable.

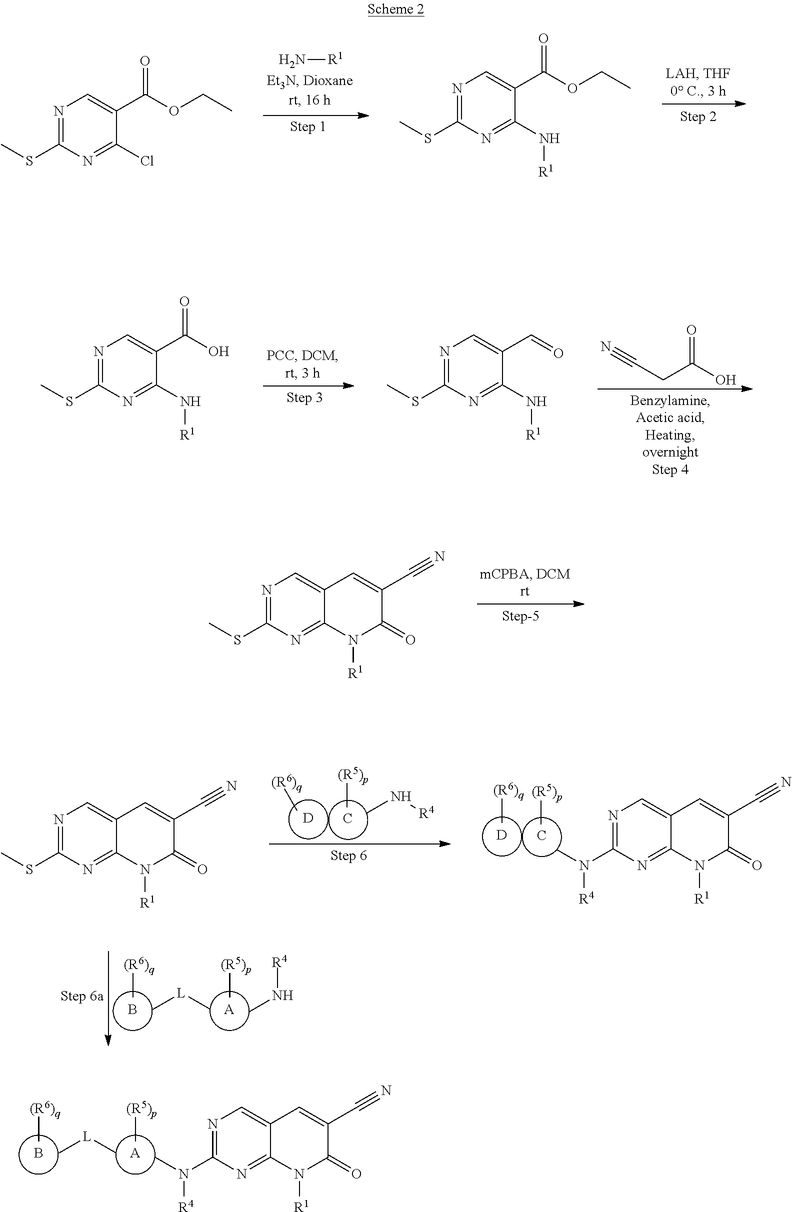

wherein A, B, C, D, L, R¹, R⁴, R⁵, R⁶; p and q are as described for Formula (K), Formula (J), Formula (I), or Formula (II).

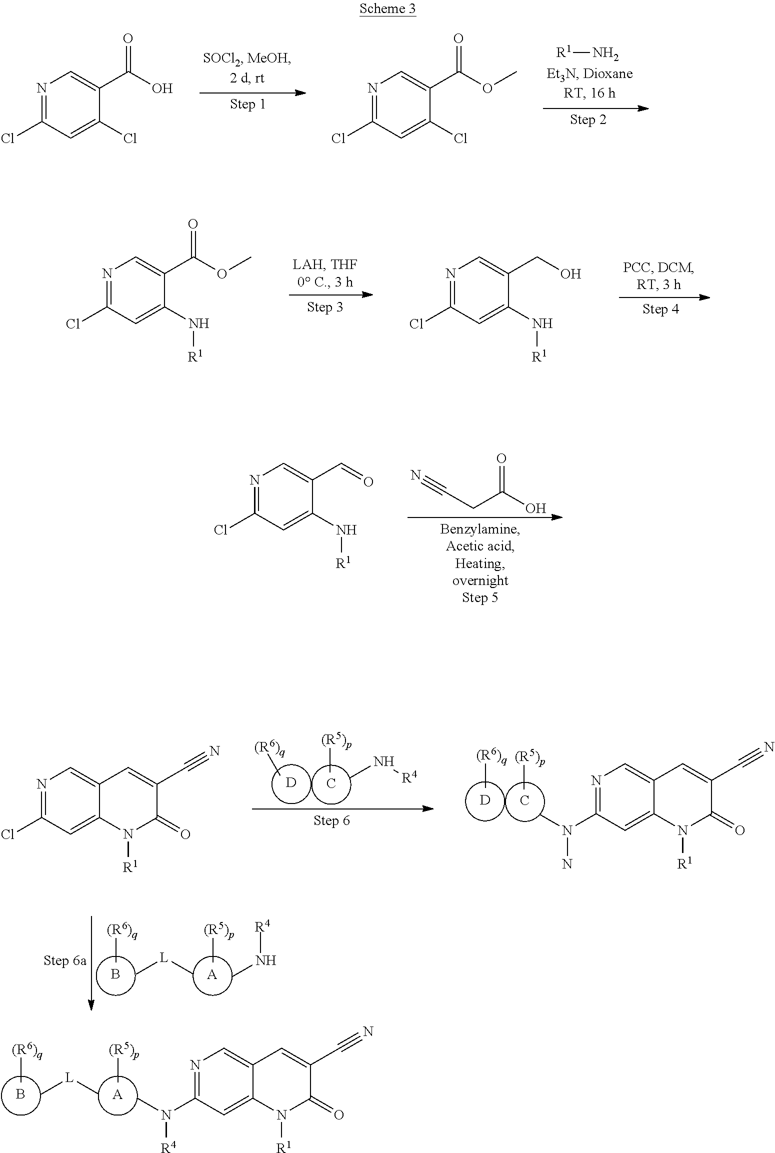

wherein A, B, C, D, L, R, R⁴, R, R⁶; p and q are as described for Formula (K), Formula (J), Formula (I), or Formula (II).

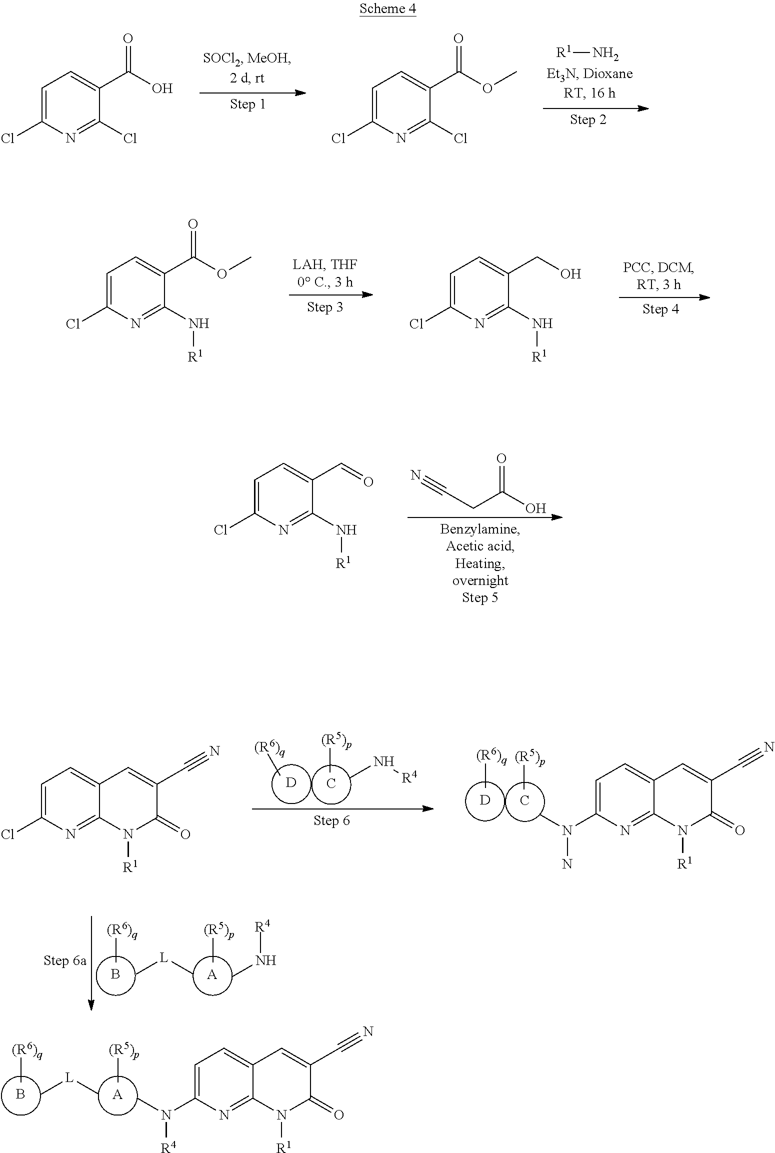

wherein A, B, C, D, L, R, R⁴, R, R⁶; p and q are as described for Formula (K), Formula (J), Formula (I), or Formula (II).

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays. In some embodiments of the methods detailed herein, the methods comprise administration of a compound detailed herein, or a salt thereof, as a monotherapy.

Provided herein is a method of treating a disease in an individual comprising administering an effective amount of a compound of Formula (J), Formula (I), Formula (II), (I-A), (I-B1) to (I-B22), (I-C1) to (I-C23) or any embodiment, variation or aspect thereof (collectively, a compound of Formula (J), Formula (I), Formula (II), (I-A), (I-B1) to (I-B22), (I-C₁) to (I-C23)) or the present compounds or the compounds detailed or described herein) or a pharmaceutically acceptable salt thereof, to the individual. Further provided herein is a method of treating a proliferative disease in an individual, comprising administering an effective amount of the compound of Formula (J), Formula (I), Formula (II), (I-A), (I-B1) to (I-B22), (I-C1) to (I-C23), or a pharmaceutically acceptable salt thereof, to the individual. Also provided herein is a method of treating cancer in an individual comprising administering an effective amount of the compound of Formula (J), Formula (I), Formula (II), (I-A), (I-B1) to (I-B22B22), (I-C1) to (I-C23) or a pharmaceutically acceptable salt thereof, to the individual. In some embodiments, the compound is administered to the individual according to a dosage and/or method of administration described herein. It is to be understood that the above-mentioned methods of treating diseases are applicable to Formula (K) or any related formulae where applicable, to the same extent as is described for Formula (J).

In some embodiments, the cancer in the individual has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of one or more of CDK1, CDK2, CDK4, CDK6 and CDK9. In some embodiments, the cancer in the individual has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of CDK4/6 and one or more of CDK1, CDK2, and CDK9.

In some embodiments, there is provided a method of treating a cancer in an individual, comprising (a) selecting the individual for treatment based on (i) the presence of phosphorylation of the retinoblastoma (Rb) protein in the cancer, or (ii) presence of mutations or amplification or overexpression of CDK4 or CDK6 in the cancer, and administering an effective amount of the compound of Formula (J), Formula (I), Formula (II), (I-A), (I-B1) to (I-B22), (I-C₁) to (I-C23), or a pharmaceutically acceptable salt thereof, to the individual. In some embodiments, the cancer is assayed for the expression of phosphorylated Rb. In some embodiments, the cancer is assayed for the expression of CDK4 or CDK6. In some embodiments, the CDK4 or CDK6 gene of the cancer is sequenced to detect the one or more mutations or amplifications. In some embodiments, the CDK4 or CDK6 gene is sequenced by biopsying the cancer and sequencing the CDK4 or CDK6 gene from the biopsied cancer. In some embodiments, the CDK4 or CDK6 gene is sequenced by sequencing circulating-tumor DNA (ctDNA) from the individual. It is to be understood that the above-mentioned methods of treating cancer are applicable to Formula (K) or any related formulae where applicable, to the same extent as is described for Formula (J).

In some embodiments, provided herein is a method of using a compound of Formula (J), Formula (I), Formula (II), (I-A), (I-B1) to (I-B22), (I-C1) to (I-C23) or any embodiment in the manufacture of a medicament for treatment of a disease. In some embodiments, provided herein is a method of using a compound of Formula (J), Formula (I), Formula (II), (I-A), (I-B1) to (I-B22), (I-C1) to (I-C23) or any embodiment in the manufacture of a medicament for treatment of cancer. It is to be understood that the above-mentioned methods of using a compound of Formula (J) are equally applicable to Formula (K) or any related formulae where applicable.

In some embodiments, a compound of Formula (J), Formula (I), Formula (II), (I-A), (I-B1) to (I-B22), (I-C1) to (I-C23) or a salt thereof is used to treat an individual having a proliferative disease, such as cancer as described herein. In some embodiments, the individual is at risk of developing a proliferative disease, such as cancer. In some of these embodiments, the individual is determined to be at risk of developing cancer based upon one or more risk factors. In some of these embodiments, the risk factor is a family history and/or gene associated with cancer. It is to be understood that the above-mentioned uses of a compound of Formula (J) are equally applicable to Formula (K) or any related formulae where applicable.

The present compounds or salts thereof are believed to be effective for treating a variety of diseases and disorders. For example, in some embodiments, the present compositions may be used to treat a proliferative disease, such as cancer. In some embodiments the cancer is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma, cutaneous T cell lymphoma, Hodgkin's lymphoma, non-Hodgkin's lymphoma, cancer of the adrenal cortex, ACTH-producing tumors, lung cancer, including small cell carcinoma and nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

In some embodiments, the cancer is defined by a molecular characteristic. In some embodiments, the cancer is an estrogen receptor-positive breast cancer. In some embodiments, the breast cancer is triple negative breast cancer. In some embodiments, the cancer is a KRAS-mutant non-small cell lung cancer. In some embodiments, the cancer is mantle cell lymphoma defined by a translocation involving CCND1 resulting in cyclin D1 overexpression.

In some embodiments, the compounds and compositions described herein cause G-S cell cycle arrest in a cell (such as a cancer cell). In some embodiments, the cancer cell is a cancer cell from any of the cancer types described herein. In some embodiments, arrested cells enter a state of apoptosis. In some embodiments, arrested cells enter a state of senescence. In some embodiments, provided herein is a method of causing G₁-S checkpoint arrest in a cell comprising administering an effective amount of the compound of Formula (J), Formula (I), Formula (II), (I-A), (I-B1) to (I-B22), (I-C1) to (I-C23) or a pharmaceutically acceptable salt thereof, to the cell. In some embodiments, the G₁-S cell cycle arrest occurs in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, the G₁-S cell cycle arrest occurs in up to about 99%, up to about 98%, up to about 97%, up to about 96%, up to about 95%, up to about 90%, up to about 85%, or up to about 80% of cells in the cell population. It is to be understood that the above-mentioned compounds comprise compounds of Formula (K) or any related formulae where applicable, to the same extent as is described for Formula (J).

In some embodiments, provided herein is a method of inducing senescence in a cell comprising administering an effective amount of the compound of Formula (J), Formula (I), Formula (II), (I-A), (I-B1) to (I-B22), (I-C1) to (I-C23) or a pharmaceutically acceptable salt thereof, to the cell. In some embodiments, senescence is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, senescence is induced in up to about 99%, up to about 98%, up to about 97%, up to about 96%, up to about 95%, up to about 90%, up to about 85%, or up to about 80% of cells in the cell population. It is to be understood that the above-mentioned methods of inducing senescence in a cell are applicable to Formula (K) or any related formulae where applicable, to the same extent as is described for Formula (J).

In some embodiments, provided herein is a method of inducing apoptosis in a cell comprising administering an effective amount of the compound of Formula (J), Formula (I), Formula (II), (I-A), (I-B1) to (I-B22), (I-C1) to (I-C23)) or a pharmaceutically acceptable salt thereof, to the cell. In some embodiments, apoptosis is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, apoptosis is induced in up to about 99%, up to about 98%, up to about 97%, up to about 96%, up to about 95%, up to about 90%, up to about 85%, or up to about 80% of cells in the cell population. It is to be understood that the above-mentioned methods of inducing apoptosis in a cell are applicable to Formula (K) or any related formulae where applicable, to the same extent as is described for Formula (J).

In some embodiments, provided herein is a method of inhibiting CDK4 or CDK6 in a cell comprising administering an effective amount of the compound of Formula (J), Formula (I), Formula (II), (I-A), (I-B1) to (I-B22), (I-C1) to (I-C23) or a pharmaceutically acceptable salt thereof, to the cell. In some embodiments, CDK4 or CDK6 is inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some embodiments, CDK4 or CDK6 is inhibited up to about 99%, up to about 98%, up to about 97%, up to about 96%, up to about 95%, up to about 90%, up to about 85%, up to about 80%, up to about 70%, or up to about 60%. In some embodiments, the activity of CDK4 or CDK6 is measured according to a kinase assay. It is to be understood that the above-mentioned methods of inducing inhibiting CDK4 or CDK6 in a cell are applicable to Formula (K) or any related formulae where applicable, to the same extent as is described for Formula (J).

In some embodiments, provided herein is a method of inhibiting one or more of CDK1, CDK2, CDK4, CDK6, and CDK9 in a cell comprising administering an effective amount of the compound of Formula (J), Formula (I), Formula (II), (I-A), (I-B1) to (I-B22), (I-C1) to (I-C23) or a pharmaceutically acceptable salt thereof, to the cell. In some embodiments, one or more of CDK1, CDK2, CDK4, CDK6, and CDK9 is inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some embodiments, one or more of CDK1, CDK2, CDK4, CDK6, and CDK9 is inhibited up to about 99%, up to about 98%, up to about 97%, up to about 96%, up to about 95%, up to about 90%, up to about 85%, up to about 80%, up to about 70%, or up to about 60%. In some embodiments, the activity of one or more of CDK1, CDK2, CDK4, CDK6, and CDK9 is measured according to a kinase assay. It is to be understood that the above-mentioned methods of inhibiting one or more of CDK1, CDK2, CDK4, CDK6, and CDK9 in a cell are applicable to Formula (K) or any related formulae where applicable, to the same extent as is described for Formula (J).

In some embodiments, provided herein is a method of inhibiting CDK4 or CDK6 comprising contacting CDK4 or CDK6 with an effective amount of the compound of Formula (J), Formula (I), Formula (II), (I-A), (I-B1) to (I-B22), (I-C1) to (I-C23) or a pharmaceutically acceptable salt thereof. In some embodiments, the compound of Formula (J), Formula (I), Formula (II), (I-A), (I-B1) to (I-B22), (I-C1) to (I-C23) or a pharmaceutically acceptable salt thereof binds to CDK4 or CDK6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (J), Formula (I), Formula (II), (I-A), (I-B1) to (I-B22), (I-C1) to (I-C23) or a pharmaceutically acceptable salt thereof binds to CDK4 or CDK6 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay. It is to be understood that the above-mentioned methods of inhibiting CDK4 or CDK6 are applicable to Formula (K) or any related formulae where applicable, to the same extent as is described for Formula (J).

In some embodiments, provided herein is a method of inhibiting one or more of CDK1, CDK2, CDK4, CDK6, and CDK9 comprising contacting one or more of CDK1, CDK2, CDK4, CDK6, and CDK9 with an effective amount of the compound of Formula (J), Formula (I), Formula (II), (I-A), (I-B1) to (I-B22), (I-C1) to (I-C23) or a pharmaceutically acceptable salt thereof. In some embodiments, the compound of Formula (J), Formula (I), Formula (II), (I-A), (I-B1) to (I-B22), (I-C1) to (I-C23) or a pharmaceutically acceptable salt thereof binds to one or more of CDK1, CDK2, CDK4, CDK6, and CDK9 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (J), Formula (I), Formula (II), (I-A), (I-B1) to (I-B22), (I-C1) to (I-C23) or a pharmaceutically acceptable salt thereof binds to one or more of CDK1, CDK2, CDK4, CDK6, and CDK9 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay. It is to be understood that the above-mentioned methods of inhibiting one or more of CDK1, CDK2, CDK4, CDK6, and CDK9 are applicable to Formula (K) or any related formulae where applicable, to the same extent as is described for Formula (J).

In some embodiments, provided herein is a method of modulating CDK4/6 in an individual, comprising administering to the individual a compound of Formula (J), Formula (I), Formula (II), (I-A), (I-B1) to (I-B22), (I-C1) to (I-C23) or a pharmaceutically acceptable salt thereof, or a salt thereof. In some embodiments, provided herein is a method of modulating CDK4 and CDK 6 in an individual, comprising administering to the individual a compound of Formula (J), Formula (I), Formula (II), (I-A), (I-B1) to (I-B22), (I-C1) to (I-C23) or a pharmaceutically acceptable salt thereof, or a salt thereof. In some embodiments, provided herein is a method of modulating CDK4/6 and one or more of CDK1, CDK2, and CDK9 in an individual, comprising administering to the individual a compound detailed herein, or a salt thereof. In some embodiments, provided herein is a method of modulating CDK4 and CDK 6 and one or more of CDK1, CDK2, and CDK9 in an individual, comprising administering to the individual a compound detailed herein, or a salt thereof. In some embodiments, the compound of Formula (J), Formula (I), Formula (II), (I-A), (I-B1) to (I-B22), (I-C1) to (I-C23) or a pharmaceutically acceptable salt thereof binds to one or more of CDK4/6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (J), Formula (I), Formula (II), (I-A), (I-B1) to (I-B22), (I-C1) to (I-C23) or a pharmaceutically acceptable salt thereof binds to one or more of CDK4 and CDK6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (J), Formula (I), Formula (II), (I-A), (I-B1) to (I-B22), (I-C1) to (I-C23) or a pharmaceutically acceptable salt thereof binds to one or more of CDK1, CDK2, CDK4, CDK6, and CDK9 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay. It is to be understood that the above-mentioned methods of modulating CDK4/6 in an individual are applicable to Formula (K) or any related formulae where applicable, to the same extent as is described for Formula (J).

In one embodiment, the compound or a salt thereof may enhance the antitumour immunity by increasing the functional capacity of tumour cells to present antigen or by reducing the immunosuppressive T_{Reg} population by suppressing their proliferation.

In some embodiments, provided herein is a method of inhibiting the proliferation of a cell, comprising contacting the cell with an effective amount of the compound of Formula (J), Formula (I), Formula (II), (I-A), (I-B1) to (I-B22), (I-C1) to (I-C23) or a pharmaceutically acceptable salt thereof. In some embodiments, the compound of Formula (J), Formula (I), Formula (II), (IA-), (I-B1) to (I-B22), (I-C1) to (I-C23) or a pharmaceutically acceptable salt thereof is effective in inhibiting the proliferation of the cell with an EC₅₀ of less than 5 μM, less than 2 μM, less than 1 μM, less than 900 nM, less than 800 nM, less than 700 nM, less than 600 nM, less than 500 nM, less than 400 nM, less than 300 nM, less than 200 nM, less than 100 nM, or less than 50 nM. In some embodiments, the compound of Formula (J), Formula (I), Formula (II), (I-A), (I-B1) to (I-B22), (I-C1) to (I-C23) or a pharmaceutically acceptable salt is effective in inhibiting the proliferation of the cell with an EC₅₀ between 10 nM and 20 nM, between 20 nM and 50 nM, between 50 nM and 100 nM, between 100 nM and 500 nM, between 500 nM and 1 μM, between 1 μM and 2 μM, or between 2 μM and 5 μM. In some embodiments, the EC₅₀ is measured according to a cell proliferation assay. It is to be understood that the above-mentioned methods of inhibiting the proliferation of a cell are applicable to Formula (K) or any related formulae where applicable, to the same extent as is described for Formula (J).

### Combination Therapy

As provided herein, the presently disclosed compounds or a salt thereof may affect the immune system. Accordingly, the present compounds or a salt thereof may be used in combination with other anti-cancer agents or immunotherapies. In some embodiments, provided herein is a method of treating a disease in an individual comprising administering an effective amount of a compound of Formula (J), Formula (I), Formula (II), (I-A), (I-B1) to (I-B22), (I-C1) to (I-C23), or any embodiment, variation or aspect thereof (collectively, a compound of Formula (J), Formula (I), Formula (II), (I-A), (I-B1) to (I-B22), (I-C1) to (I-C23) or the present compounds or the compounds detailed or described herein) or a pharmaceutically acceptable salt thereof, and an additional therapeutic agent to the individual. In some embodiments, the second therapeutic agent is a cancer immunotherapy agent or an endocrine therapy agent or a chemotherapeutic agent. In some embodiments, the disease is a proliferative disease such as cancer. It is to be understood that the above-mentioned methods of treating a disease in an individual are applicable to Formula (K) or any related formulae where applicable, to the same extent as is described for Formula (J).

In some embodiments, the additional therapeutic agent is a cancer immunotherapy agent. In some embodiments, the additional therapeutic agent is an immunostimulatory agent. In some embodiments, the additional therapeutic agent targets a checkpoint protein (for example an immune checkpoint inhibitor). In some embodiments, the additional therapeutic agent is effective to stimulate, enhance or improve an immune response against a tumor.

In another aspect provided herein is a combination therapy for the treatment of a disease, such as cancer. In some embodiments, a method of treating a disease in an individual is provided, the method comprising administering an effective amount of Formula (J), Formula (I), Formula (II), (I-A), (I-B1) to (I-B22), (I-C1) to (I-C23), or any embodiment, variation or aspect thereof (collectively, a compound of Formula (J), Formula (I), Formula (II), (I-A), (I-B1) to (I-B22), (I-C1) to (I-C23), or the present compounds or the compounds detailed or described herein) or a pharmaceutically acceptable salt thereof, in combination with a radiation therapy. It is to be understood that the above-mentioned methods of treating a disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23) or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of an endocrine therapy agent. In some embodiments, the endocrine therapy is antiestrogen therapy. In some embodiments, the endocrine therapy is a selective estrogen receptor degrader (SERD, such as fulvestrant). In some embodiments, the endocrine therapy is an aromatase inhibitor (such as letrozole). In some embodiments, the combination of a CDK4/6 inhibitor and endocrine therapy causes enhancement of G1-S cell-cycle arrest. In some embodiments, the combination of a CDK4/6 inhibitor and endocrine therapy causes enhanced entry into a senescent state. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the endocrine therapy agent. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the endocrine therapy agent. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a second chemotherapeutic agent. In some embodiments, the chemotherapeutic agent is another kinase inhibitor. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the second chemotherapeutic agent. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the second chemotherapeutic agent. It is to be understood that the above-mentioned methods of treating disease in an individual are applicable to Formula (K) or any related formulae where applicable, to the same extent as is described for Formula (J).

Examples of chemotherapeutic agents that can be used in combination with Formula (J), Formula (I), Formula (II), (I-A), (I-B1) to (I-B22), (I-C1) to (I-C23) or a pharmaceutically acceptable salt thereof include DNA-targeted agents, a DNA alkylating agent (such as cyclophosphamide, mechlorethamine, chlorambucil, melphalan, dacarbazine, or nitrosoureas), a topoisomerase inhibitor (such as a Topoisomerase I inhibitor (e.g., irinotecan or topotecan) or a Topoisomerase II inhibitor (e.g., etoposide or teniposide)), an anthracycline (such as daunorubicin, doxorubicin, epirubicin, idarubicin, mitoxantrone, or valrubicin), a histone deacetylase inhibitor (such as vorinostat or romidepsin), a bromodomain inhibitor, other epigenetic inhibitors, a taxane (such as paclitaxel or docetaxel), a kinase inhibitor (such as bortezomib, erlotinib, gefitinib, imatinib, vemurafenib, vismodegib, ibrutinib), an anti-angiogenic inhibitor, a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine), or a platinum-based chemotherapeutic agent (such as cisplatin, carboplatin, or oxaliplatin), pemetrexed, or a combination thereof. In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a kinase inhibitor (such as bortezomib, erlotinib, gefitinib, imatinib, vemurafenib, vismodegib, or ibrutinib). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the kinase inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the kinase inhibitor. It is to be understood that the above-mentioned chemotherapeutic agents can be used with a compound of Formula (K) or any related formulae where applicable, to the same extent as is described for a compound of Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a DNA damaging agent. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the DNA damaging agent. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the DNA damaging agent. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23)), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a DNA alkylating agent (such as cyclophosphamide, mechlorethamine, chlorambucil, melphalan, dacarbazine, or nitrosoureas). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the DNA alkylating agent. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the DNA alkylating agent. It is to be understood that the above-mentioned methods of treating disease in an individual are applicable to Formula (K) or any related formulae where applicable, to the same extent as is described for Formula (J).

In some embodiments, a method of treating a disease in an individual is provided, the method a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a topoisomerase inhibitor (such as a Topoisomerase I inhibitor (e.g., irinotecan or topotecan) or a Topoisomerase II inhibitor (e.g., etoposide or teniposide)). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the topoisomerase inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the topoisomerase inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of an anthracycline (such as daunorubicin, doxorubicin, epirubicin, idarubicin, mitoxantrone, or valrubicin). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the anthracycline. In some embodiments, Formula (J), Formula (I), Formula (II), (IA-), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the anthracycline. It is to be understood that the above-mentioned methods of treating disease in an individual are applicable to Formula (K) or any related formulae where applicable, to the same extent as is described for Formula (J).

In some embodiments, a method of treating a disease in an individual is provided, the method a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a histone deacetylase inhibitor (such as vorinostat or romidepsin). In some embodiments, Formula I or a pharmaceutically acceptable salt thereof is administered prior to, after, or simultaneously co-administered with the histone deacetylase inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the histone deacetylase inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a taxane (such as paclitaxel or docetaxel). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the taxane. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the taxane. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the nucleotide analog or precursor analog. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the nucleotide analog or precursor analog. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a platinum-based chemotherapeutic agent (such as cisplatin, carboplatin, or oxaliplatin). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the platinum-based chemotherapeutic agent. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the platinum-based chemotherapeutic agent. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of pemetrexed. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the pemetrexed. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the pemetrexed. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a Bruton's tyrosine kinase (BTK) inhibitor. In some embodiments, Formula (J), Formula (I), Formula (II), (IA-), (I-B1) to (I-B22), (I-C1) to (I-C23) or a pharmaceutically acceptable salt thereof is administered prior to, after, or simultaneously co-administered with the BTK inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the BTK inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a PI3K or Akt inhibitor. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the PI3K or Akt inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the PI3K or Akt inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23)), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a DNA damage repair (DDR) pathway inhibitor. In some embodiments, Formula (J), Formula (I), Formula (II), (IA-), (I-B1) to (I-B22), (I-C1) to (I-C23) or a pharmaceutically acceptable salt thereof is administered prior to, after, or simultaneously co-administered with the DDR pathway inhibitor. In some embodiments, Formula (J), Formula (I), Formula (II), (IA-), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the DDR pathway inhibitor. Examples of inhibitors of the DDR pathway include poly(ADP-ribose) polymerase (PARP) inhibitors (such as olaparib, rucaparib, niraparib, or talazoparib), ataxia telangiectasia mutated (ATM) protein inhibitors, ataxia telangiectasia and Rad3-related (ATR) protein inhibitors, checkpoint kinase 1 (Chk1) inhibitors, or combinations thereof. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a PARP inhibitor (such as olaparib, rucaparib, niraparib, or talazoparib). In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the PARP inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the PARP inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23)), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of an ATM protein inhibitor. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the ATM protein inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the ATM protein inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of an ATR protein inhibitor. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the ATR protein inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the ATR protein inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of an Chk1 inhibitor. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the Chk1 inhibitor. In some embodiments, Formula (J), Formula (I), Formula (II), (I-A), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the Chk1 inhibitor. It is to be understood that the above-mentioned methods of treating disease in an individual are applicable to Formula (K) or any related formulae where applicable, to the same extent as is described for Formula (J).

In some embodiments, a method of treating a disease in an individual is provided, the method comprising (a) administering an effective amount of Formula (J), Formula (I), Formula (II), (I-A), (I-B1) to (I-B22), (I-C1) to (I-C23)), or any embodiment, variation or aspect thereof (collectively, Formula (J), Formula (I), Formula (II), (I-A), (I-B1) to (I-B22), (I-C1) to (I-C23)) or a pharmaceutically acceptable salt thereof, and (b) administering an effective amount of a further CDK4/6 inhibitor. In some embodiments, Formula (J), Formula (I), Formula (II), (I-A), (I-B1) to (I-B22), (I-C1) to (I-C23) or a pharmaceutically acceptable salt thereof is administered prior to, after, or simultaneously co-administered with the further CDK4/6 inhibitor. In some embodiments, Formula (J), Formula (I), Formula (II), (IA-), (I-B1) to (I-B22), (I-C1) to (I-C23) or a pharmaceutically acceptable salt thereof is administered 1 or more hours (such as 2 or more hours, 4 or more hours, 8 or more hours, 12 or more hours, 24 or more hours, or 48 or more hours) prior to or after the further CDK4/6 inhibitor. It is to be understood that the above-mentioned methods of treating disease in an individual are applicable to Formula (K) or any related formulae where applicable, to the same extent as is described for Formula (J).

In another aspect, provided herein is a combination therapy in which a compound of Formula (J), Formula (I), Formula (II), (I-A), (I-B1) to (I-B22), (I-C1) to (I-C23), or a salt thereof is coadministered (which may be separately or simultaneously) with one or more additional agents that are effective in stimulating immune responses to thereby further enhance, stimulate or upregulate immune responses in a subject. For example, provided is a method for stimulating an immune response in a subject comprising administering to the subject a compound of Formula (J), Formula (I), Formula (II), (I-A), (I-B1) to (I-B22), (I-C1) to (I-C23), or a salt thereof and one or more immunostimulatory antibodies, such as an anti-PD-1 antibody, an anti-PD-L1 antibody and/or an anti-CTLA-4 antibody, such that an immune response is stimulated in the subject, for example to inhibit tumor growth. In one embodiment, the subject is administered a compound of formula Formula (J), Formula (I), Formula (II), (I-A), (I-B1) to (I-B22), (I-C1) to (I-C23) or a salt thereof and an anti-PD-1 antibody. In another embodiment, the subject is administered a compound of Formula (J), Formula (I), Formula (II), (I-A), (I-B1) to (I-B22), (I-C1) to (I-C23), or a salt thereof and an anti-PD-L1 antibody. In yet another embodiment, the subject is administered a compound of Formula (J), Formula (I), Formula (II), (I-A), (I-B1) to (I-B22), (I-C1) to (I-C23) or a salt thereof and an anti-CTLA-4 antibody. In another embodiment, the immunostimulatory antibody (e.g., anti-PD-1, anti-PD-L1 and/or anti-CTLA-4 antibody) is a human antibody. Alternatively, the immunostimulatory antibody can be, for example, a chimeric or humanized antibody (e.g., prepared from a mouse anti-PD-1, anti-PD-L1 and/or anti-CTLA-4 antibody). It is to be understood that the above-mentioned combination therapy is applicable to Formula (K) or any related formulae where applicable, to the same extent as is described for Formula (J).

In one embodiment, the present disclosure provides a method for treating a proliferative disease (e.g., cancer), comprising administering a compound of Formula (J), Formula (I), Formula (II), (I-A), (I-B1) to (I-B22), (I-C1) to (I-C23), or a salt thereof and an anti-PD-1 antibody to a subject. In further embodiments, a compound of Formula (J), Formula (I), Formula (II), (I-A), (I-B1) to (I-B22), (I-C1) to (I-C23) or a salt thereof is administered at a subtherapeutic dose, the anti-PD-1 antibody is administered at a subtherapeutic dose, or both are administered at a subtherapeutic dose. In another embodiment, the present disclosure provides a method for altering an adverse event associated with treatment of a hyperproliferative disease with an immunostimulatory agent, comprising administering a compound of Formula (J), Formula (I), Formula (II), (I-A), (I-B1) to (I-B22), (I-C1) to (I-C23), or a salt thereof and a subtherapeutic dose of anti-PD-1 antibody to a subject. In certain embodiments, the subject is human. In certain embodiments, the anti-PD-1 antibody is a human sequence monoclonal antibody. It is to be understood that the above-mentioned methods of treating proliferative disease are applicable to Formula (K) or any related formulae where applicable, to the same extent as is described for Formula (J).

In one embodiment, the present invention provides a method for treating a hyperproliferative disease (e.g., cancer), comprising administering a compound of Formula (J), Formula (I), Formula (II), (I-A), (I-B1) to (I-B22), (I-C1) to (I-C23), or a salt thereof and an anti-PD-L1 antibody to a subject. In further embodiments, a compound of Formula (J), Formula (I), Formula (II), (I-A), (I-B1) to (I-B22), (I-C1) to (I-C23) or a salt thereof is administered at a subtherapeutic dose, the anti-PD-L1 antibody is administered at a subtherapeutic dose, or both are administered at a subtherapeutic dose. In another embodiment, the present invention provides a method for altering an adverse event associated with treatment of a hyperproliferative disease with an immunostimulatory agent, comprising administering a compound of Formula (J), Formula (I), Formula (II), (I-A), (I-B1) to (I-B22), (I-C1) to (I-C23), or a salt thereof and a subtherapeutic dose of anti-PD-L1 antibody to a subject. In certain embodiments, the subject is human. In certain embodiments, the anti-PD-L1 antibody is a human sequence monoclonal antibody. It is to be understood that the above-mentioned methods of treating hyperproliferative disease are applicable to Formula (K) or any related formulae where applicable, to the same extent as is described for Formula (J).

In certain embodiments, the combination of therapeutic agents discussed herein can be administered concurrently as a single composition in a pharmaceutically acceptable carrier, or concurrently as separate compositions each in a pharmaceutically acceptable carrier. In another embodiment, the combination of therapeutic agents can be administered sequentially. For example, an anti-CTLA-4 antibody and a compound of Formula (J), Formula (I), Formula (II), (I-A), (I-B1) to (I-B22), (I-C1) to (I-C23), or a salt thereof can be administered sequentially, such as anti-CTLA-4 antibody being administered first and a compound of Formula (J), Formula (I), Formula (II), (I-A), (I-B1) to (I-B22), (I-C1) to (I-C23), or a salt thereof second, or a compound of Formula (J), Formula (I), Formula (II), (I-A), (I-B1) to (I-B22), (I-C1) to (I-C23), or a salt thereof being administered first and anti-CTLA-4 antibody second. Additionally or alternatively, an anti-PD-1 antibody and a compound of Formula (J), Formula (I), Formula (II), (I-A), (I-B1) to (I-B22), (I-C1) to (I-C23), or a salt thereof can be administered sequentially, such as anti-PD-1 antibody being administered first and a compound of Formula (J), Formula (I), Formula (II), (I-A), (I-B1) to (I-B22), (I-C1) to (I-C23), or a salt thereof second, or a compound of Formula (J), Formula (I), Formula (II), (I-A), (I-B1) to (I-B22), (I-C1) to (I-C23), or a salt thereof being administered first and anti-PD-1 antibody second. Additionally or alternatively, an anti-PD-L1 antibody and a compound of Formula (J), Formula (I), Formula (II), (I-A), (I-B1) to (I-B22), (I-C1) to (I-C23), or a salt thereof can be administered sequentially, such as anti-PD-L1 antibody being administered first and a compound of Formula (J), Formula (I), Formula (II), (I-A), (I-B1) to (I-B22), (I-C1) to (I-C23), or a salt thereof second, or a compound of Formula (J), Formula (I), Formula (II), (I-A), (I-B1) to (I-B22), (I-C1) to (I-C23), or a salt thereof being administered first and anti-PD-L1 antibody second. It is to be understood that the above-mentioned combinations of therapeutic agents are applicable to Formula (K) or any related formulae where applicable, to the same extent as is described for Formula (J).

Furthermore, if more than one dose of the combination therapy is administered sequentially, the order of the sequential administration can be reversed or kept in the same order at each time point of administration, sequential administrations can be combined with concurrent administrations, or any combination thereof.

Optionally, the combination of a compound of Formula (J), Formula (I), Formula (II), (I-A), (I-B1) to (I-B22), (I-C1) to (I-C23), or a salt thereof can be further combined with an immunogenic agent, such as cancerous cells, purified tumor antigens (including recombinant proteins, peptides, and carbohydrate molecules), cells, and cells transfected with genes encoding immune stimulating cytokines. It is to be understood that the above-mentioned combinations are applicable to Formula (K) or any related formulae where applicable, to the same extent as is described for Formula (J).

A compound of Formula (J), Formula (I), Formula (II), (I-A), (I-B1) to (I-B22), (I-C₁) to (I-C23), or a salt thereof can also be further combined with standard cancer treatments. For example, a compound of Formula (J), Formula (I), Formula (II), (I-A), (I-B1) to (I-B22), (I-C₁) to (I-C23), or a salt thereof can be effectively combined with chemotherapeutic regimens. In these instances, it is possible to reduce the dose of other chemotherapeutic reagent administered with the combination of the instant disclosure. Other combination therapies with a compound of Formula (J), Formula (I), Formula (II), (I-A), (I-B1) to (I-B22), (I-C1) to (I-C23)), or a salt thereof include radiation, surgery, or hormone deprivation. Angiogenesis inhibitors can also be combined with a compound of Formula (J), Formula (I), Formula (II), (I-A), (I-B1) to (I-B22), (I-C1) to (I-C23), or a salt thereof. Inhibition of angiogenesis leads to tumor cell death, which can be a source of tumor antigen fed into host antigen presentation pathways. It is to be understood that the above-mentioned combinations are applicable to Formula (K) or any related formulae where applicable, to the same extent as is described for Formula (J).

In another example, a compound of Formula (J), Formula (I), Formula (II), (I-A), (I-B1) to (I-B22), (I-C1) to (I-C23)), or a salt thereof can be used in conjunction with anti-neoplastic antibodies. By way of example and not wishing to be bound by theory, treatment with an anti-cancer antibody or an anti-cancer antibody conjugated to a toxin can lead to cancer cell death (e.g., tumor cells) which would potentiate an immune response mediated by CTLA-4, PD-1, PD-L1 or a compound of Formula (J), Formula (I), Formula (II), (I-A), (I-B1) to (I-B22), (I-C₁) to (I-C23), or a salt thereof. In an exemplary embodiment, a treatment of a hyperproliferative disease (e.g., a cancer tumor) can include an anti-cancer antibody in combination with a compound of Formula (J), Formula (I), Formula (II), (I-A), (I-B1) to (I-B22), (I-C1) to (I-C23) or a salt thereof and anti-CTLA-4 and/or anti-PD-1 and/or anti-PD-L1 antibodies, concurrently or sequentially or any combination thereof, which can potentiate anti-tumor immune responses by the host. Other antibodies that can be used to activate host immune responsiveness can be further used in combination with a compound of Formula (J), Formula (I), Formula (II), (I-A), (I-B1) to (I-B22), (I-C1) to (I-C23) or a salt thereof. It is to be understood that the above-mentioned combinations are applicable to Formula (K) or any related formulae where applicable, to the same extent as is described for Formula (J).

In some embodiments, a compound of Formula (J), Formula (I), Formula (II), (I-A), (I-B1) to (I-B22), (I-C1) to (I-C23), or a salt thereof can be combined with an anti-CD73 therapy, such as an anti-CD73 antibody. In some embodiments, a compound of Formula (K) or a salt thereof can be combined with an anti-CD73 therapy, such as an anti-CD73 antibody.

In yet further embodiments, the compound of Formula (J), Formula (I), Formula (II), (I-A), (I-B1) to (I-B22), (I-C1) to (I-C23), or a salt thereof is administered in combination with another CDK4 or CDK6 inhibitor or other CDK inhibitor. In yet further embodiments, the compound of Formula (K) or a salt thereof is administered in combination with another CDK4 or CDK6 inhibitor or other CDK inhibitor.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form.

Also provided are articles of manufacture comprising a compound of the disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed.

### Kits

The present disclosure further provides kits for carrying out the methods of the invention, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### Selected Embodiments

Embodiment 1. A compound of Formula (J):

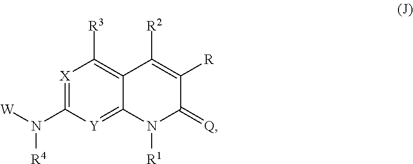

or a salt thereof, wherein:

X is CR^{a} or N, wherein R^{a} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²;

Y is CR^{b} or N, wherein R^{b} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹², provided that at least one of X and Y is N;

Q is O or S;

W is

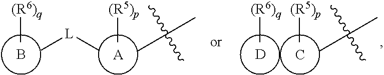

wherein:
A is C₃-C₆ cycloalkyl, 4- to 7-membered heterocyclyl, 5- to 7-membered heteroaryl, or phenyl, each of which is independently optionally substituted by R⁵,
L is a bond, —CR¹¹R¹²—, —O—, —S—, —S(O)₂—, —C(O)—, —NR¹⁰—, —S(O)₂NR¹⁰—, or —NR¹⁰S(O)₂—,
B is hydrogen, C₃-C₆ cycloalkyl, 3- to 10-membered heterocyclyl, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶,
C is C₃-C₆ cycloalkyl, 5- to 7-membered heterocyclyl, 5- to 7-membered heteroaryl, or phenyl, each of which is independently optionally substituted by R⁵, wherein C is fused to D, and
D is C₃-C₆ cycloalkyl, 3- to 7-membered heterocyclyl, 5- to 7-membered heteroaryl, or phenyl, each of which is independently optionally substituted by R⁶;

R is —CN or C₁-C₆ haloalkyl;

R¹ is C₁-C₆ alkyl, C₃-C₆ cycloalkyl, 3- to 12-membered heterocyclyl, 5- to 10-membered heteroaryl, C₆-C₁₄ aryl, —(C₁-C₃ alkylene)(C₃-C₆ cycloalkyl), —(C₁-C₃ alkylene)(3- to 12-membered heterocyclyl), —C(O)R¹⁰, —(C₁-C₃ alkylene)(5- to 10-membered heteroaryl), or —(C₁-C₃ alkylene)(C₆-C₁₄ aryl), each of which is independently optionally substituted by halogen, oxo, —OR¹³, —NR¹³R¹⁴, —C(O)R¹³, —CN, C₃-C₈ cycloalkyl, or C₁-C₆ alkyl optionally substituted by oxo, —OH or halogen;

R² and R³ are each independently hydrogen, C₁-C₆ alkyl, C₃-C₆ cycloalkyl, C₁-C₆ haloalkyl, C₁-C₆ alkoxy, C₁-C₆ haloalkoxy, halogen, —CN, —C(O)R¹⁰, or —C(O)NR¹¹R¹²;

R⁴ is hydrogen or C₁-C₆ alkyl;

each R⁵ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen;
each R⁶ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen,
or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each optionally substituted by C₁-C₆ alkyl;

R¹⁰ is independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR¹⁵, —NR¹⁵R¹⁶, or C₁-C₆ alkyl optionally substituted by halogen, —OH or oxo;

R¹¹ and R¹² are each independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR, —NR¹⁵R¹⁶ or C₁-C₆ alkyl optionally substituted by halogen, —OH or oxo,
or R¹¹ and R¹² are taken together with the atom to which they attached to form a 3- to 6-membered heterocyclyl optionally substituted by halogen, oxo, or C₁-C₆ alkyl optionally substituted by halogen;

R¹³ and R¹⁴ are each independently hydrogen, —OH, C₁-C₆ alkoxy, or C₁-C₆ alkyl, wherein the C₁-C₆ alkyl of R¹³ and R¹⁴ are optionally substituted by halogen, —OR, —NR¹⁵R¹⁶, or oxo,
or R¹³ and R¹⁴ are taken together with the atom to which they attached to form a 3- to 6-membered heterocyclyl optionally substituted by halogen, oxo or C₁-C₆ alkyl optionally substituted by halogen or oxo;

R¹⁵ and R¹⁶ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo,
or R¹⁵ and R¹⁶ are taken together with the atom to which they attached to form a 3- to 6-membered heterocyclyl optionally substituted by halogen, oxo or C₁-C₆ alkyl optionally substituted by oxo or halogen;

p and q are each independently 0, 1, 2, 3 or 4.

Embodiment 2. The compound of embodiment 1, or a salt thereof, wherein X is CR^{a}.
Embodiment 3. The compound of embodiment 1, or a salt thereof, wherein X is N.
Embodiment 4. The compound of any one of embodiments 1-3, or a salt thereof, wherein Y is CR^{b}.
Embodiment 5. The compound of any one of embodiments 1-3, or a salt thereof, wherein Y is N.
Embodiment 6. The compound of any one of embodiments 1-5, or a salt thereof, wherein Q is O.
Embodiment 7. The compound of any one of embodiments 1-6, or a salt thereof, wherein R is —CN.
Embodiment 8. The compound of any one of embodiments 1-7, or a salt thereof, wherein the compound is of Formula (I),

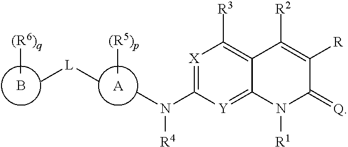

Embodiment 9. The compound of any one of embodiments 1-7, or a salt thereof, wherein the compound is of Formula (II),

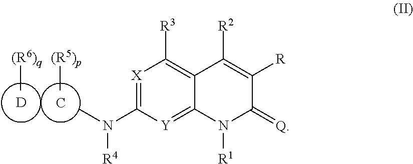

Embodiment 10. The compound of any one of embodiments 1-9, or a salt thereof, wherein R¹ is C₁-C₆ alkyl, C₃-C₆ cycloalkyl, 3- to 12-membered heterocyclyl, —(C₁-C₃ alkylene)(C₆-C₁₄ aryl), C₆-C₁₄ aryl, or —(C₁-C₃ alkylene)(C₃-C₆ cycloalkyl), each of which is independently optionally substituted by halogen, —OR¹³, or C₁-C₆ alkyl optionally substituted by oxo, —OH, or halogen.
Embodiment 11. The compound of any one of embodiments 1-10, or a salt thereof, wherein R² is hydrogen.
Embodiment 12. The compound of any one of embodiments 1-11, or a salt thereof, wherein R³ is hydrogen.
Embodiment 13. The compound of any one of embodiments 1-12, or a salt thereof, wherein R⁴ is hydrogen,
Embodiment 14. The compound of any one of embodiments 1-8 and 10-13, or a salt thereof, wherein the compound is of any one of Fomulae (I-B1) to (I-B22),

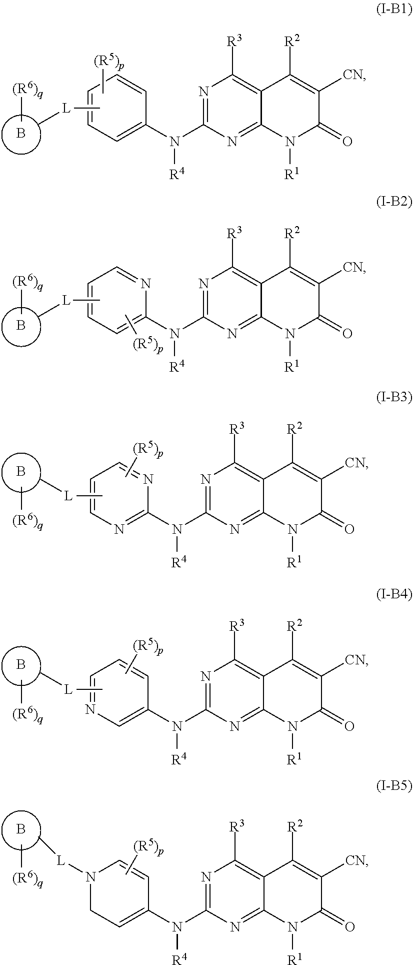

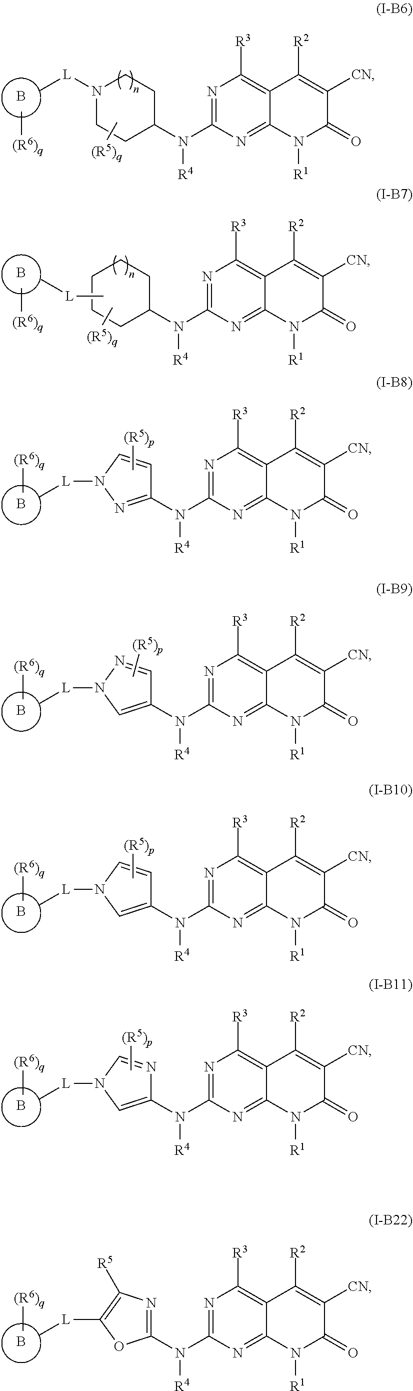

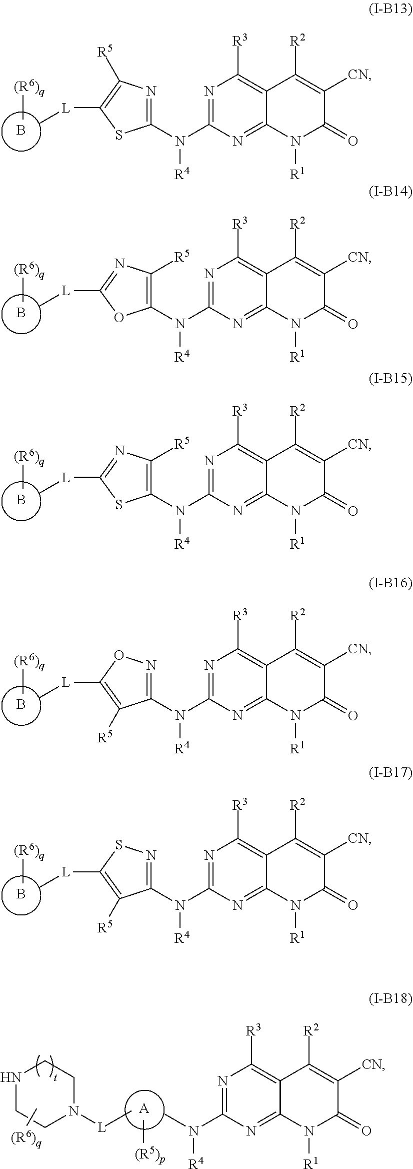

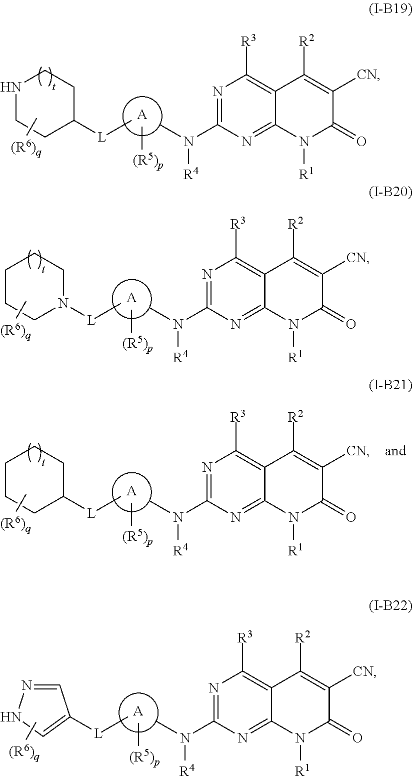

Embodiment 15. The compound of any one of embodiments 1-8 and 9-14, or a salt thereof, wherein L is a bond.
Embodiment 16. The compound of any one of embodiments 1-8 and 10-13, or a salt thereof, wherein the compound is of any one of Formulae (I-C1) to (I-C23):

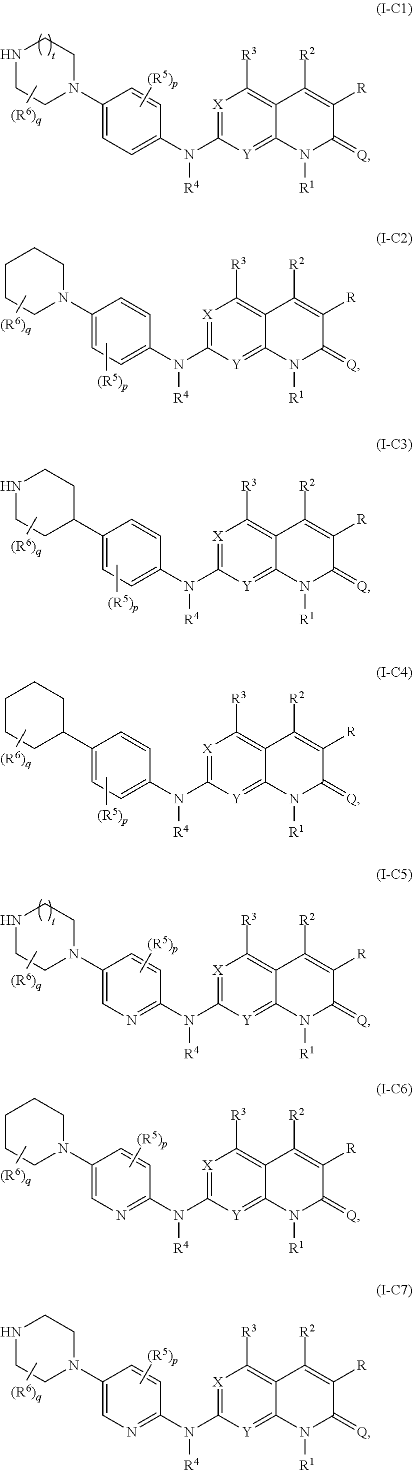

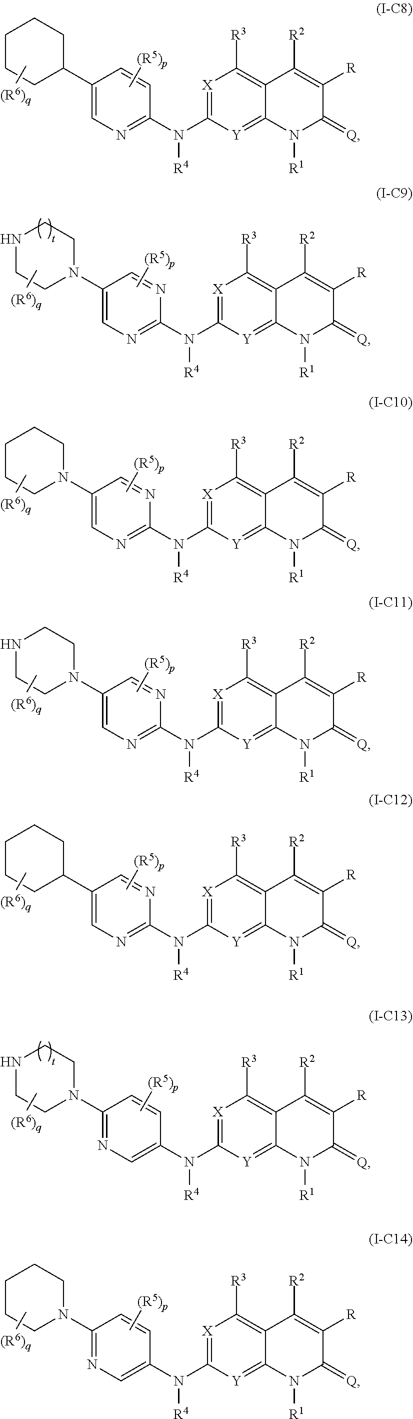

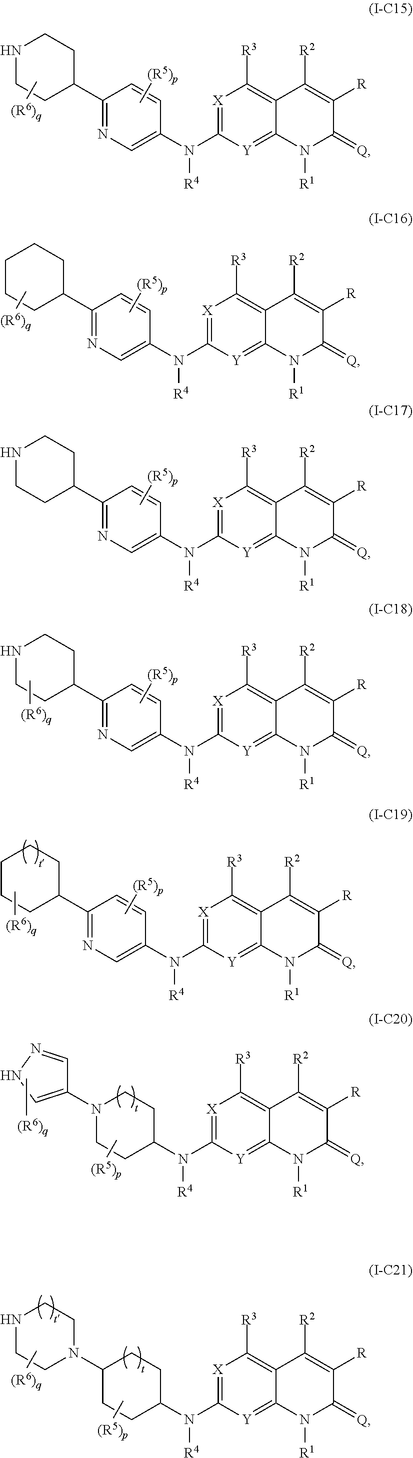

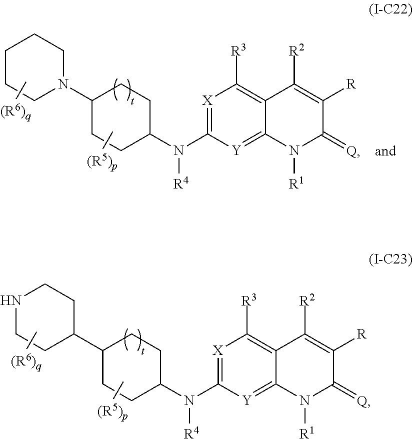

wherein t and t′ are each independently 0, 1, 2, or 3.
Embodiment 17. The compound of any one of embodiments 1-7 and 9-13, or a salt thereof, wherein C, D, R⁵, and R⁶ together form a moiety selected from the group consisting of:

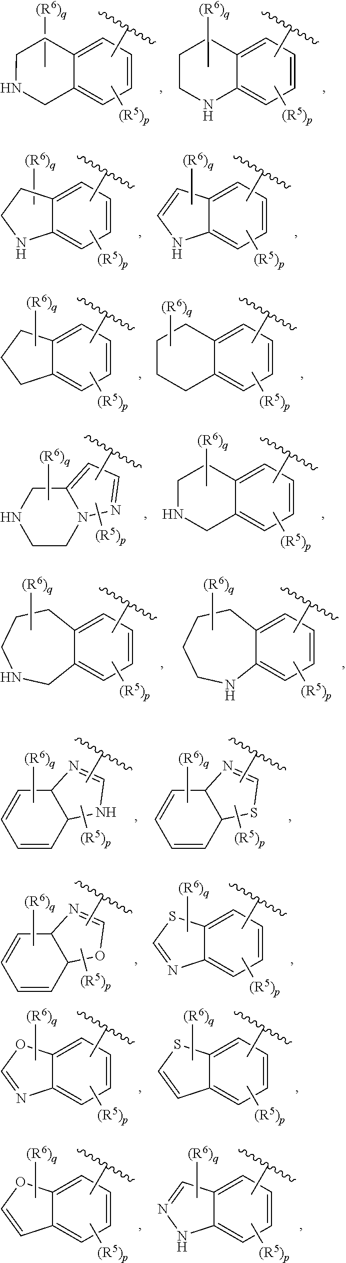

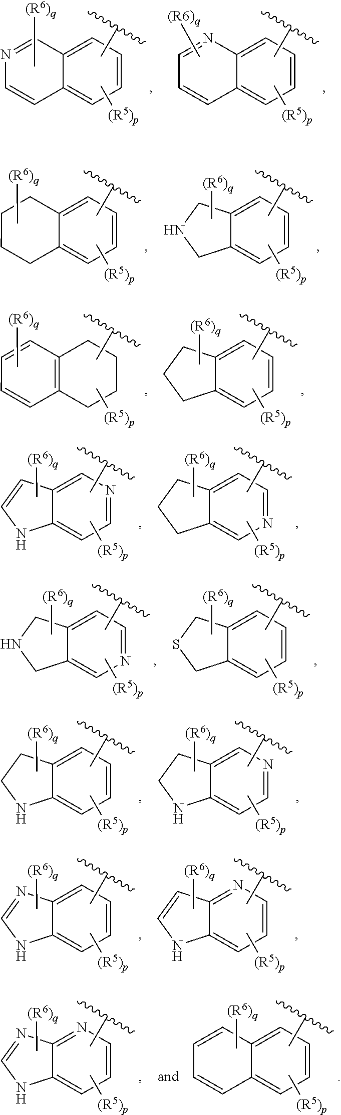

Embodiment 18. The compound of any one of embodiments 1-17, or a salt thereof, wherein each R⁵ is independently —S(O)₂R⁰, —S(O)₂NR¹¹R¹², —C(O)NR¹¹R¹², —(C₁-C₃ alkylene)OR¹⁰, 3- to 12-membered heterocyclyl, —(C₁-C₃ alkylene)NR¹¹R¹², halogen, C₁-C₆ alkyl, —OR¹⁰, or oxo, each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen.
Embodiment 19. The compound of any one of embodiments 1-18, or a salt thereof, wherein each R⁶ is independently C₁-C₆ alkyl, oxo, —OR¹⁰, —(C₁-C₃ alkylene)NR¹¹R¹², C₃-C₆ cycloalkyl, 3- to 12-membered heterocyclyl, —S(O)₂NR¹¹R¹², —NR¹¹R¹², —C(O)R¹⁰, —(C₁-C₃ alkylene)C(O)NR¹¹R¹², —S(O)₂R¹⁰, —(C₁-C₃ alkylene)(C₃-C₆ cycloalkyl), —(C₁-C₃ alkylene)(3- to 12-membered heterocyclyl), or —(C₁-C₃ alkylene)OR¹⁰, each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen;
or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each independently optionally substituted by C₁-C₆ alkyl.
Embodiment 20. A compound selected from the group of the embodiments in Tables 1, or a salt thereof.
Embodiment 21. A pharmaceutical composition comprising the compound of any one of embodiments 1-20, or a pharmaceutically acceptable salt thereof, and a pharmaceutically acceptable carrier.
Embodiment 22. A method of treating a cancer in an individual in need thereof comprising administering to the individual a therapeutically effective amount of a compound of any one of embodiments 1-20, or a pharmaceutically acceptable salt thereof.
Embodiment 23. The method of embodiment 22, where the cancer is a breast cancer, brain cancer, colorectal cancer, lung cancer, gastric cancer, liver cancer, leukemia, lymphoma, mantle cell lymphoma, melanoma, ovarian cancer, pancreatic cancer, prostate cancer, adult hematopoietic or solid tumor, or pediatric tumor.
Embodiment 24. The method of embodiment 22 or 23, further comprising administering a radiation therapy to the individual.
Embodiment 25. The method of any one of embodiments 22-24, further comprising administering to the individual a therapeutically effective amount of a second therapeutic agent.
Embodiment 26. The method of embodiment 25, wherein the second therapeutic agent is a cancer immunotherapy agent, an endocrine therapy agent, or a chemotherapeutic agent.
Embodiment 27. The method of embodiment 25 or 26, wherein the second therapeutic agent is a cancer immunotherapy.
Embodiment 28. The method of any one of embodiment 25-27, wherein the second therapeutic agent is an anti-PD-1 antibody.
Embodiment 29. The method of embodiment 26, wherein the endocrine therapy agent is an antiestrogen therapy, a selective estrogen receptor degrader (SERD), or an aromatase inhibitor.
Embodiment 30. The method of embodiment 26, wherein the chemotherapeutic agent is a DNA alkylating agent, a platinum-based chemotherapeutic agent, a taxane, a BTK inhibitor, a PI3K inhibitor, another kinase inhibitor, or a DNA damage repair (DDR) pathway inhibitor.
Embodiment 31. The method of any one of embodiments 22-30, wherein the cancer comprises a mutated or overexpressed CDK gene.
Embodiment 32. The method of any one of embodiments 22-31, comprising selecting the individual for treatment based on (i) the presence of one or more mutations or amplifications of the CDK4 or CDK6 or other CDK gene in the cancer, (ii) overexpression of CDK4 or CDK6 or other CDK protein in the cancer, (iii) amplification or overexpression of the genes encoding cyclins, (iv) loss of endogenous INK4 inhibitors by gene deletion, mutation, or promoter hypermethylation, (v) other genetic events leading to overactivity of CDK4 or CDK6 or other CDK, or (vi) phosphorylation of retinoblastoma (Rb) protein in the cancer.
Embodiment 33. A method of arresting the G₁-S checkpoint in a cell, comprising administering a compound of any one of embodiments 1-20, or a salt thereof, to the cell.
Embodiment 34. A method of inducing senescence in a cell, comprising administering a compound of any one of embodiments 1-20, or a salt thereof, to the cell.
Embodiment 35. A method of inducing apoptosis in a cell, comprising administering a compound of any one of embodiments 1-20, or a salt thereof, to the cell.
Embodiment 36. A method of inhibiting CDK4 or CDK6 in a cell, comprising administering a compound of any one of embodiments 1-20, or a salt thereof, to the cell.
Embodiment 37. A method of inhibiting CDK4 or CDK6, comprising contacting CDK4 or CDK6 with a compound of any one of embodiments 1-20, or a salt thereof.
Embodiment 38. The method of claim 37, wherein the inhibitor binds to CDK4 or CDK6 with an IC₅₀ of less than 1 μM according to a kinase assay.
Embodiment 39. Use of a compound of any one of embodiments 1-20, or a salt thereof, in the manufacture of a medicament for treatment of cancer.
Embodiment 40. A kit comprising a compound of any one of embodiments 1-20, or a salt thereof.

The invention can be further understood by reference to the following examples, which are provided by way of illustration and are not meant to be limiting.

### EXAMPLES

### Synthetic Examples

### Example—S1: Synthesis of 8-cyclopentyl-7-oxo-2-((1-oxo-1,2,3,4-tetrahydroisoquinolin-7-yl)amino)-7,8-dihydropyrido[2,3-d]pyrimidine-6-carbonitrile (Compound No. 6)

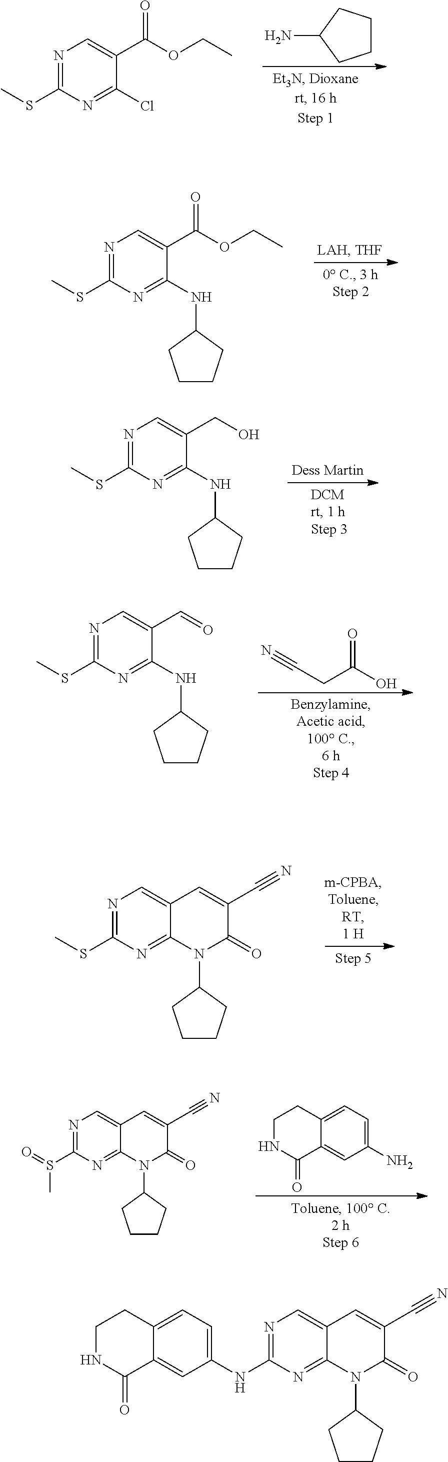

Step-1: Synthesis of ethyl 4-(cyclopentylamino)-2-(methylthio)pyrimidine-5-carboxylate: To a stirred solution of ethyl 4-chloro-2-(methylthio)pyrimidine-5-carboxylate (5 g, 21.55 mmol, 1.0 equiv) in dioxane (50 mL), was added triethylamine (6.05 mL, 43.1 mmol, 2.0 equiv) and cyclopentanamine (2.198 g, 25.82 mmol, 1.2 equiv) at room temperature. Stirred the reaction mixture for 16 h at room temperature. Progress of the reaction was monitored by TLC and LCMS. After completion of the reaction, the reaction mixture was diluted with water (100 mL) and extracted with ethyl acetate (100 mL×2), organic layer was washed with water (100 mL) and brine solution (100 mL), dried over anhydrous sodium sulphate. Concentrated under reduced pressure to obtain desired product. LCMS: 282 [M+H]⁺

Step-2: Synthesis of (4-(cyclopentylamino)-2-(methylthio)pyrimidin-5-yl)methanol: To a stirred solution of ethyl 4-(cyclopentylamino)-2-(methylthio)pyrimidine-5-carboxylate (7 g, 24.91 mmol, 1.0 equiv) in THF (100 mL), was added portion wise LAH (2.836 g, 74.73 mmol) at 0° C. The reaction mixture was allowed to stir at room temperature for 3 h. Progress of the reaction was monitored by TLC and LCMS. After completion of the reaction, the reaction mixture was quenched with saturated solution of sodium sulphate dropwise at 0° C. Obtained residue was filtered through cealite bed. Filtrate was extracted with ethyl acetate (100 mL×2). The combined organic layer was washed with water (100 mL) and brine solution (100 mL), dried over anhydrous sodium sulphate and concentrated under reduced pressure to obtain desired product. LCMS: 240 [M+H]⁺

Step-3: Synthesis of 4-(cyclopentylamino)-2-(methylthio)pyrimidine-5-carbaldehyde: To a stirred solution of (4-(cyclopentylamino)-2-(methylthio)pyrimidin-5-yl)methanol (5 g, 20.92 mmol, 1.0 equiv) in DCM (50 mL), was added pyridinium chlorochromate (8.995 g, 41.84 mmol, 2.0 equiv) at 0° C. The reaction mixture was allowed to stirred at room temperature for 1 h. Progress of the reaction was monitored by TLC and LCMS. After completion of the reaction, workup done by filtration of reaction mass through cealite pad by and celite bed was washed by DCM (50 mL×2) filtrate was diluted with water (100 mL), and extracted with DCM (100 mL×2). The combined organic layer was washed with sodium bicarbonate solution (100 mL) and brine solution (100 mL), dried over anhydrous sodium sulphate and concentrated under reduced pressure to obtain desired product. LCMS: 238 [M+H]⁺

Step-4: Synthesis of 8-cyclopentyl-2-(methylthio)-7-oxo-7,8-dihydropyrido[2,3-d]pyrimidine-6-carbonitrile: To a stirred solution of 4-(cyclopentylamino)-2-(methylthio)pyrimidine-5-carbaldehyde (5 g, 21.09 mmol, 1.0 equiv.) in Acetic acid (50 mL), was added cyano acetic acid (2.151 g, 25.31 mmol, 1.2 equiv) and Benzyl amine (0.250 g, 2.109 mmol, 0.1 equiv). The reaction mixture was allowed to stir at 100° C. for 6 h (refluxed). Progress of the reaction was monitored by TLC and LCMS. After completion of the reaction, the reaction mixture was diluted with water (50 mL) and extracted with ethyl acetate (50 mL×2). The combined organic layer was washed with water (50×2 mL) and brine solution (50 mL). Organic layer was dried over anhydrous sodium sulphate and concentrated under reduced pressure to obtain crude compound, which was purified by normal phase combi-flash to obtain desired product. LCMS: 287 [M+H]⁺

Step-5: Synthesis of 8-cyclopentyl-2-(methylsulfinyl)-7-oxo-7,8-dihydropyrido[2,3-d]pyrimidine-6-carbonitrile: To a stirred solution of obtain 8-cyclopentyl-2-(methylthio)-7-oxo-7,8-dihydropyrido[2,3-d]pyrimidine-6-carbonitrile (500 mg, 1.74 mmol, 1 equiv) in toluene (5 mL) was added m-CPBA (330 mg, 1.92 mmol, 1.4 equiv) at room temperature. Stirred the reaction for 1 h. Progress of the reaction was monitored by TLC and LCMS. After completion of the reaction, the reaction mixture was diluted with sodium bicarbonate solution (10 mL) and extracted with ethyl acetate (15 mL×2). The combined organic layer was washed with water (10 mL) and sodium bicarbonate solution (50 mL) brine solution (50 mL), dried over anhydrous sodium sulphate and concentrated under reduced pressure to obtain desired product. LCMS: 303 [M+H]+; ¹H NMR: (DMSO-d₆, 400 MHz): δ 9.28 (s, 1H), 8.92 (s, 1H), 5.78-5.94 (m, 1H), 2.95 (s, 3H), 2.14-2.26 (m, 2H), 2.01-2.14 (m, 2H), 1.82-1.93 (m, 2H), 1.55-1.69 (m, 2H).

Step—6: Synthesis of 8-cyclopentyl-7-oxo-2-((1-oxo-1,2,3,4-tetrahydroisoquinolin-7-yl)amino)-7,8-dihydropyrido[2,3-d]pyrimidine-6-carbonitrile: To a suspension of 8-cyclopentyl-2-(methylsulfinyl)-7-oxo-7,8-dihydropyrido[2,3-d]pyrimidine-6-carbonitrile (50 mg, 0.16 mmol) in toluene (3 mL) was added 7-amino-3,4-dihydroisoquinolin-1(2H)-one (53 mg, 0.33 mmol) The resultant reaction mixture was stir at 100° C. for 2 h. Progress of the reaction was monitored by LCMS. Product precipitates out was filtered. Residue was triturated from methanol to afford desired product. LCMS: 401 (M+1); ¹H NMR (400 MHz, DMSO-d₆) δ 10.64 (br. s., 1H), 8.86 (s, 1H), 8.58 (s, 1H), 7.97 (br. s., 1H), 7.61 (br. s., 1H), 7.30 (d, J=8.33 Hz, 1H), 5.96 (d, J=7.89 Hz, 1H), 3.37 (br. s., 2H), 2.88 (br. s., 2H), 2.13 (br. s., 2H), 1.87 (br. s., 4H), 1.65 (br. s., 2H)

### Example—S2: Synthesis of 8-((1r,4r)-4-methylcyclohexyl)-7-oxo-2-((1-oxo-1,2,3,4-tetrahydroisoquinolin-7-yl)amino)-7,8-dihydropyrido[2,3-d]pyrimidine-6-carbonitrile (Compound No. 7)

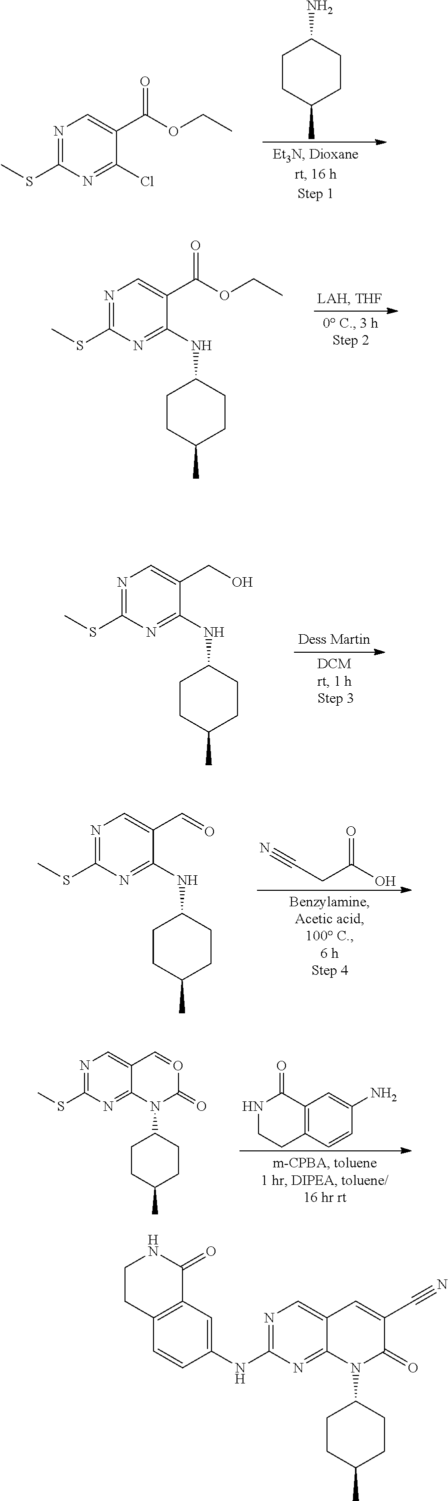

Step—1: Synthesis of ethyl 4-(((1r,4r)-4-methylcyclohexyl)amino)-2-(methylthio)pyrimidine-5-carboxylate: To a stirred solution of ethyl 4-chloro-2-(methylthio)pyrimidine-5-carboxylate (1 g, 4.3 mmol, 1 eqiv) in Dioxane (10 mL), was added Et₃N (1.2 mL, 8.6 mmol, 2 eqiv) and (1r,4r)-4-methylcyclohexan-1-amine (0.585 g, 5.17 mmol, 1.2 eqiv) at room temperature. The resultant reaction mixture was stirred at room temperature for 16 h. Progress of the reaction was monitored by TLC and LCMS. After completion of the reaction, the reaction mixture was diluted with water (30 mL) and extracted with ethyl acetate (100 mL×2). Organic layer was washed with water (100 mL), brine solution (100 mL). Organic layer was dried over anhydrous sodium sulphate and concentrated under reduced pressure to obtain desired product. LCMS: 310 [M+H]⁺

Step—2: Synthesis of (4-(((1r,4r)-4-methylcyclohexyl)amino)-2-(methylthio)pyrimidin-5-yl)methanol: To a stirred solution of ethyl 4-(((1r, 4r)-4-methylcyclohexyl) amino)-2-(methylthio) pyrimidine-5-carboxylate (1.3 g, 4.20 mmol, 1 eqiv) in THF (30 mL), was added LAH (0.558 g, 14.70 mmol, 4 eqiv) at 0° C. The reaction mixture was allowed to stir at 0° C. for 3 h. Progress of the reaction was monitored by TLC and LCMS. After completion of the reaction, the reaction mixture was quenched with saturated solution of sodium sulphate at 0° C. and extracted with ethyl acetate (100 mL×3). Organic layer was washed with water (100 mL) and brine solution (100 mL). Organic layer was dried over anhydrous sodium sulphate and concentrated under reduced pressure to obtain desired product. LCMS: 268.1 [M+H]⁺

Step—3: Synthesis of 4-(((1r,4r)-4-methylcyclohexyl)amino)-2-(methylthio)pyrimidine-5-carbaldehyde: To a stirred solution of (4-(((1r, 4r)-4-methylcyclohexyl) amino)-2-(methylthio) pyrimidin-5-yl) methanol (1.1 g, 4.11 mmol, 1 eqiv) in DCM (11 mL), was added Dess-Martin periodinane (0.558 mg, 14.70 mmol, 1.8 eqiv) at 0° C. The reaction mixture was allowed to stirr at RT for 1 h. Progress of the reaction was monitored by TLC and NMR. After completion of the reaction, the reaction mixture was quenched with the mixture of saturated solution of sodium thiosulphate: saturated solution of sodium bicarbonate (1: 1, 100 mL), and extracted with ethyl acetate (150 mL×2). Organic layer was washed with water (100 mL) and brine solution (100 mL). Organic layer was dried over anhydrous sodium sulphate and concentrated under reduced pressure to obtain desired product. LCMS: 266 [M+H]⁺

Step—4: Synthesis of 8-((1r,4r)-4-methylcyclohexyl)-2-(methylthio)-7-oxo-7,8-dihydropyrido[2,3-d]pyrimidine-6-carbonitrile: To a stirred solution of 4-(((1r, 4r)-4-methylcyclohexyl) amino)-2-(methylthio) pyrimidine-5-carbaldehyde (500 mg, 1.886 mmol, 1 eqiv) in Acetic acid (10 mL), was added cyano acetic acid (192.3 mg, 2.263 mmol, 1.2 eqiv) and benzyl amine (0.02 mL, 0.188, 0.1 eqiv). The reaction mixture was heated to 100° C. for 6 h under reflux condition. Progress of the reaction was monitored by TLC and LCMS. After completion of the reaction, the reaction mixture was diluted with water (30 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain desired product. LCMS: 315.2 [M+H]⁺

Step—5: Synthesis of 8-((1r,4r)-4-methylcyclohexyl)-7-oxo-2-((1-oxo-1,2,3,4-tetrahydroisoquinolin-7-yl)amino)-7,8-dihydropyrido[2,3-d]pyrimidine-6-carbonitrile: To a stirred solution of compound 8-((1r,4r)-4-methylcyclohexyl)-2-(methylthio)-7-oxo-7,8-dihydropyrido[2,3-d]pyrimidine-6-carbonitrile (250 mg, 0.796 mmol) in toluene10 ml and 5 ml THF for solubility was added a meta chloro per benzoic acid 55% in aq. (164 mg, 0.955 mmol) was stirred for 60 min, LCMS was checked for formation of sulfonyl, reaction mixture was filtered concentrated, added a 7-amino-3,4-dihydroisoquinolin-1(2H)-one (128.95 mg, 0.796 mmol). Followed by addition of DIPEA (0.4 ml, 2.38 mmol) in toluene 5 ml+5 ml THF. The resultant reaction mixture was stir at room temperature for 16 h. Progress of the reaction was monitored by LCMS. Solid observed was filtered and washed with pentane (50 mL). Dried under vacuum to obtain solid crude compound, which was purified by reverse phase purification to afford desired product. LCMS: 429 [M+H]⁺; ¹H NMR (400 MHz, DMSO-d₆): δ 8.84 (s, 1H), 8.56 (s, 1H), 8.30 (s, 1H), 8.19 (s, 1H), 7.96 (br. s., 1H), 7.76 (s, 1H), 7.31 (d, J=7.89 Hz, 1H), 6.61 (br. s., 2H), 5.29 (s, 2H), 2.89 (t, J=6.36 Hz, 2H), 2.67 (br. s., 1H), 2.33 (br. s., 1H), 2.08 (d, J=8.33 Hz, 1H), 1.75 (br. s., 1H), 1.70 (br. s., 1H), 1.54 (br. s., 2H), 1.23 (br. s., 1H), 1.11 (br. s., 1H), 0.99 (d, J=6.58 Hz, 1H), 0.91 (br. s., 1H)

### Example—S3: Synthesis of 8-cyclopentyl-2-((4,4-dimethyl-1,2,3,4-tetrahydroisoquinolin-7-yl)amino)-7-oxo-7,8-dihydropyrido[2,3-d]pyrimidine-6-carbonitrile (Compound No. 8)

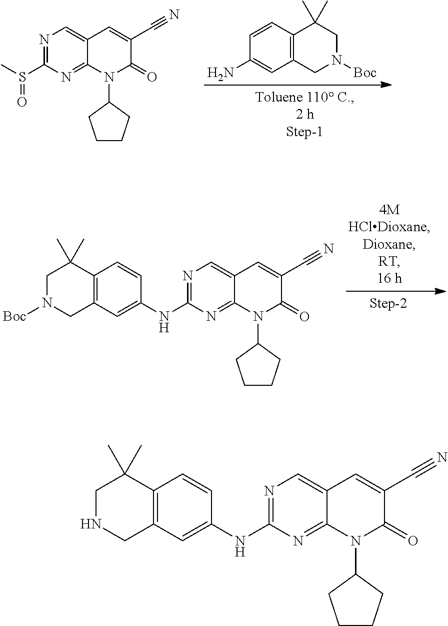 [SMILES: CS(=O)c1ncc2cc(C#N)c(=O)n(C3CCCC3)c2n1 ; CC(C)(C)OC(=O)N1Cc2cc(N)ccc2C(C)(C)C1 ; CC(C)(C)OC(=O)N1Cc2cc(Nc3ncc4cc(C#N)c(=O)n(C5CCCC5)c4n3)ccc2C(C)(C)C1 ; CC1(C)CNCc2cc(Nc3ncc4cc(C#N)c(=O)n(C5CCCC5)c4n3)ccc21]

Step—1: Synthesis of tert-butyl 7-((6-cyano-8-cyclopentyl-7-oxo-7,8-dihydropyrido[2,3-d]pyrimidin-2-yl)amino)-4,4-dimethyl-3,4-dihydroisoquinoline-2(1H)-carboxylate: To a suspension of 8-cyclopentyl-2-(methylsulfinyl)-7-oxo-7,8-dihydropyrido[2,3-d]pyrimidine-6-carbonitrile (50 mg, 0.16 mmol, 1 eqiv) in toluene (3 mL), was added tert-butyl 7-amino-4,4-dimethyl-3,4-dihydroisoquinoline-2(1H)-carboxylate (50 mg, 0.18 mmol, 1.1 eqiv). Resultant reaction mixture was stirred at 110° C. for 2 h. Reaction was monitored by LCMS. After completion of reaction, reaction mass concentrated under reduced pressure. Product was triturated from methanol to afford desired product. LCMS: 515 [M+H]⁺

Step—2: Synthesis of 8-cyclopentyl-2-((4,4-dimethyl-1,2,3,4-tetrahydroisoquinolin-7-yl)amino)-7-oxo-7,8-dihydropyrido[2,3-d]pyrimidine-6-carbonitrile: To a solution of tert-butyl 7-((6-cyano-8-cyclopentyl-7-oxo-7,8-dihydropyrido[2,3-d]pyrimidin-2-yl)amino)-4,4-dimethyl-3,4-dihydroisoquinoline-2(1H)-carboxylate (60 mg, 0.116 mmol, 1 eqiv) in dioxane (1 mL) was added to the 4M HCl in dioxane (2 mL). The resultant reaction mixture was stirred at RT for 16 h. Reaction was monitored by LCMS. After completion of reaction, filtered the solid product and washed with diethyl ether and dried under reduced pressure to afford desired product. LCMS: 415 [M+H]⁺, ¹H NMR; (400 MHz, DMSO-d₆) δ 10.73-10.47 (m, 1H), 8.86 (s, 1H), 8.60 (s, 1H), 7.61 (s, 1H), 7.57-7.44 (m, 1H), 7.20 (d, J=8.2 Hz, 1H), 7.07 (s, 1H), 5.79 (s, 1H), 4.24 (dt, J=10.6, 4.6 Hz, 4H), 3.96-3.87 (m, 1H), 3.79 (s, 2H), 3.20 (t, J=6.6 Hz, 4H), 2.19 (dq, J=14.6, 7.6, 6.7 Hz, 2H), 2.01-1.78 (m, 3H), 1.67-1.56 (m, 2H).

### Example—S4: Synthesis of 8-cyclopentyl-7-oxo-2-((2-oxo-1,2,3,4-tetrahydroquinolin-7-yl)amino)-7,8-dihydropyrido[2,3-d]pyrimidine-6-carbonitrile (Compound No. 9)

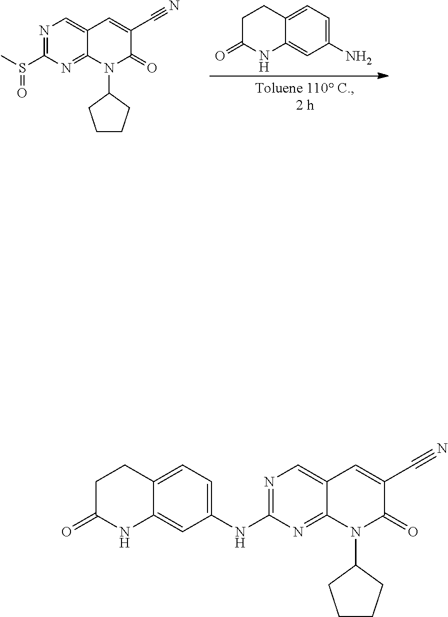 [SMILES: CS(=O)c1ncc2cc(C#N)c(=O)n(C3CCCC3)c2n1 ; Nc1ccc2c(c1)NC(=O)CC2 ; N#Cc1cc2cnc(Nc3ccc4c(c3)NC(=O)CC4)nc2n(C2CCCC2)c1=O]

To a suspension of 8-cyclopentyl-2-(methylsulfinyl)-7-oxo-7,8-dihydropyrido[2,3-d]pyrimidine-6-carbonitrile (50 mg, 0.16 mmol, 1 eqiv) in toluene (3 mL), was added 7-amino-3,4-dihydroquinolin-2(1H)-one (30 mg, 0.18 mmol, 1.1 eqiv). Resultant reaction mixture was stirred at 110° C. for 2 h. Reaction was monitored by LCMS. After completion of reaction, reaction mass concentrated under reduced pressure. Product was triturated from methanol to afford desired product. LCMS: 401 [M+H]⁺; ¹H NMR: (400 MHz, DMSO-d6) δ 10.45 (s, 1H), 10.16 (s, 1H), 8.82 (s, 1H), 8.57 (s, 1H), 7.24 (d, J=7.8 Hz, 1H), 7.15 (d, J=8.2 Hz, 1H), 7.06 (d, J=13.8 Hz, 1H), 5.87-5.68 (m, 1H), 2.85 (t, J=7.5 Hz, 2H), 2.44 (t, J=7.6 Hz, 2H), 2.16 (dt, J=15.9, 7.5 Hz, 2H), 1.90-1.73 (m, 4H), 1.56 (t, J=8.7 Hz, 2H).

### Example—S5: Synthesis of 8-cyclopentyl-7-oxo-2-((2-oxoindolin-6-yl)amino)-7,8-dihydropyrido[2,3-d]pyrimidine-6-carbonitrile (Compound No. 19)

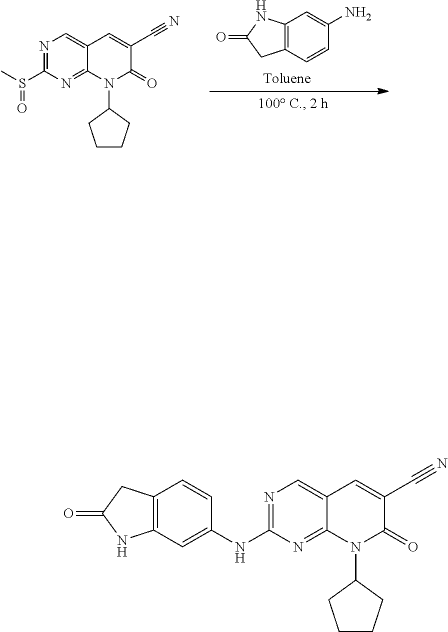 [SMILES: CS(=O)c1ncc2cc(C#N)c(=O)n(C3CCCC3)c2n1 ; Nc1ccc2c(c1)NC(=O)C2 ; N#Cc1cc2cnc(Nc3ccc4c(c3)NC(=O)C4)nc2n(C2CCCC2)c1=O]

To a stirred solution of 8-cyclopentyl-2-(methylsulfinyl)-7-oxo-7,8-dihydropyrido[2,3-d]pyrimidine-6-carbonitrile (75 mg, 0.248 mmol, 1 eqiv) in toluene (3 mL), was added 6-aminoindolin-2-one (40.48 mg, 0.273 mmol, 1.1 eqiv). The resultant reaction mixture was heated at 100° C. for 2 h. Reaction was monitored by LCMS. After completion of reaction, solid precipitate out was filtered and purified the crude product by triturated from methanol to afford desired product. LCMS: 387 [M+H]⁺; ¹H NMR: (400 MHz, DMSO-d₆) δ 10.50 (s, 1H), 8.84 (s, 1H), 8.56 (s, 1H), 7.25 (d, J=8.2 Hz, 1H), 7.18 (d, J=8.1 Hz, 1H), 5.81 (s, 1H), 3.44 (s, 2H), 2.16 (q, J=8.7, 8.2 Hz, 2H), 1.90 (s, 2H), 1.80 (dd, J=14.1, 7.0 Hz, 2H), 1.59 (dd, J=10.0, 5.3 Hz, 2H).

### Example—S6: Synthesis of 8-cyclopentyl-2-((3-((dimethylamino)methyl)-1H-indol-6-yl)amino)-7-oxo-7,8-dihydropyrido[2,3-d]pyrimidine-6-carbonitrile (Compound No. 21)

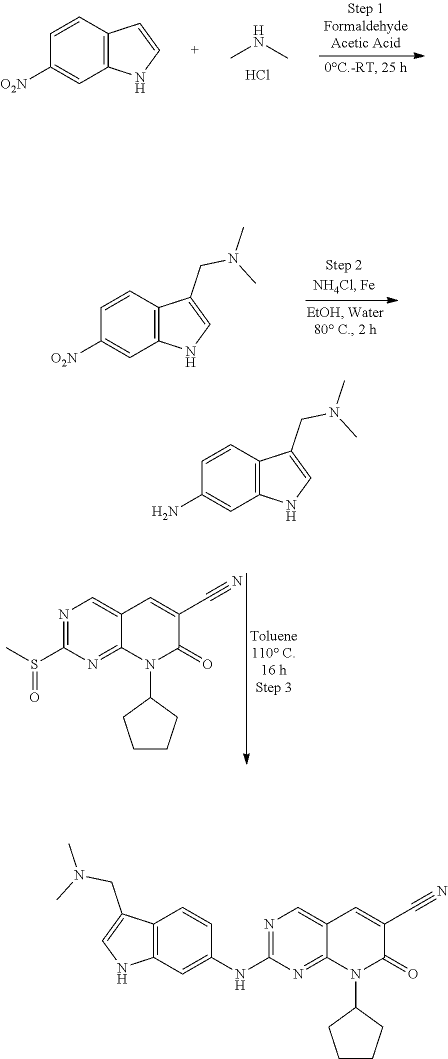

Step—1: Synthesis of N,N-dimethyl-1-(6-nitro-1H-indol-3-yl)methanamine: To a solution of dimethylamine hydrochloride (500 mg, 6.126 mmol, 2.48 eqiv) and formaldehyde (235 mg, 7.83 mmol, 3.17 eqiv) in Acetic acid (2 mL) was stirred at 0° C. for 30 min. To this solution 6-nitro-1H-indole (400 mg, 2.47 mmol, 1 eqiv) was added. Resultant reaction mixture was stirred for 24 h at RT. Reaction was monitored by LCMS. After completion of reaction, the reaction mixture was poured into 15% aqueous solution of sodium hydroxide (8 ml) at 0° C. The resultant precipitate was filtered out and washed by water and dried under reduced pressure to afford desired product. LCMS: 220 [M+H]⁺

Step—2: Synthesis of 3-((dimethylamino)methyl)-1H-indol-6-amine: To a stirred solution of N,N-dimethyl-1-(6-nitro-1H-indol-3-yl)methanamine (100 mg, 0.46 mmol, 1 eqiv) in ethanol (3 mL), was added Iron (257 mg, 4.6 mmol, 10 eqiv), ammonium chloride (246 mg, 4.6 mmol, 10 eqiv) and water (1 ml). The resultant reaction mixture was heated at 80° C. for 2 h. Reaction was monitored by TLC and LCMS. Reaction mixture was filtered through the celite. The filtrate was concentrated under reduced pressure. Aqueous layer was extracted by the ethyl acetate (5 mL). Aqueous layer was concentrated under reduced pressure and 10% solution of methanol in DCM (10 mL) was added and sonicated. Filtered the suspension and filtrate was concentrated to afford desired product. LCMS: 190 [M+H]⁺

Step—3: Synthesis of 8-cyclopentyl-2-((3-((dimethylamino)methyl)-1H-indol-6-yl)amino)-7-oxo-7,8-dihydropyrido[2,3-d]pyrimidine-6-carbonitrile: To a suspension of 8-cyclopentyl-2-(methylsulfinyl)-7-oxo-7,8-dihydropyrido[2,3-d]pyrimidine-6-carbonitrile (65.23 mg, 0.216 mmol, 1 eqiv) in toluene (4 mL), was 3-((dimethylamino)methyl)-1H-indol-6-amine (45 mg, 0.238 mmol, 1.1 eqiv). Resultant reaction mixture was stirred at 110° C. for 16 h. Reaction was monitored by LCMS. After completion of reaction, reaction mass was concentrated under reduced pressure to afford crude compound. Crude compound was purified by reverse phase HPLC to afford desired product LCMS: 428 [M+H]⁺; ¹H NMR: (400 MHz, Methanol-d4) δ 8.83 (s, 1H), 8.74 (s, 1H), 8.56 (s, 1H), 8.34 (s, 1H), 7.72 (d, J=9.1 Hz, 1H), 7.53 (s, 1H), 7.39-7.31 (m, 1H), 5.93 (s, 1H), 4.46 (s, 2H), 2.84 (s, 6H), 2.27 (d, J=11.9 Hz, 2H), 2.05 (s, 1H), 1.84 (s, 2H), 1.72 (s, 2H).

### Example—S7: Synthesis of 8-cyclopentyl-2-((3-((dimethylamino)methyl)-1-oxo-1,2,3,4 tetrahydroisoquinolin-7-yl)amino)-7-oxo-7,8-dihydropyrido[2,3-d]pyrimidine-6-carbonitrile (Compound No. 22)

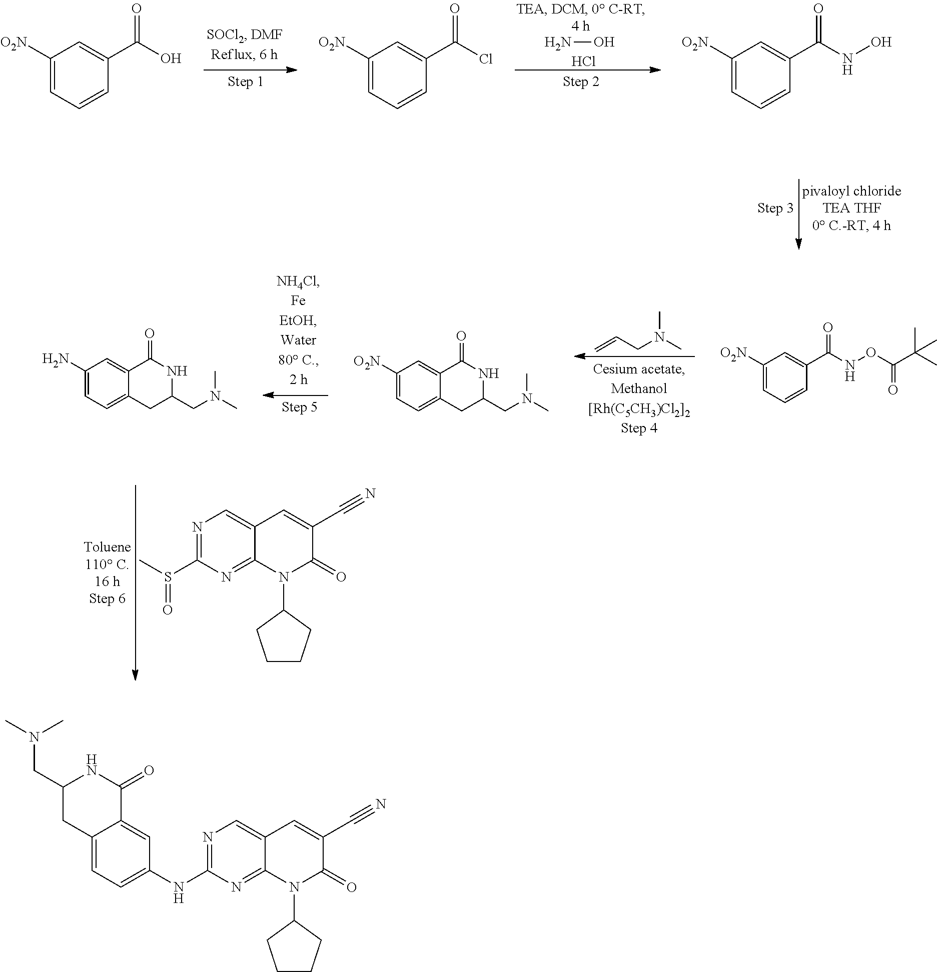

Step—1: Synthesis of 3-nitrobenzoyl chloride: To a suspension of 3-nitrobenzoic acid (10 g) in thionyl chloride (100 mL) was added DMF (0.1 mL) at room temperature. Resultant reaction mixture was refluxed for 6 h. Reaction was monitored by LCMS. After completion of reaction, reaction mass concentrated under reduced pressure to afford crude desired product, which was used directly for next step. LCMS: 182 [M+H]⁺

Step—2: Synthesis of N-hydroxy-3-nitrobenzamide: To a suspension of 3-nitrobenzoic acid 3-nitrobenzoyl chloride (10 g, 54.05 mmol, 1 eqiv) in DCM (100 mL) was added hydroxylamine hydrochloride (7.4 g, 108.1 mmol, 2 eqiv), triethylamine (21.84 g, 216.2 mmol, 4 eqiv) at 0° C. Resultant reaction mixture was stirred for 4 h at RT. Reaction was monitored by TLC and LCMS. After completion of reaction, water (50 mL) was added to the reaction mixture and extracted with DCM (100 mL×2). Combined all organic layers and washed with water (100 mL×3) and brine solution (150 mL). The organic layer was dried over anhydrous sodium sulphate and concentrated under reduced pressure to obtain desired product. LCMS: 183 [M+H]⁺

Step—3: Synthesis of 3-nitro-N-(pivaloyloxy)benzamide: To a suspension of N-hydroxy-3-nitrobenzamide (3 g, 16.48 mmol, 1 eqiv) in THF (50 mL) was added pivaloyl chloride (2.96 g, 24.72 mmol, 1.5 eqiv), triethylamine (5.18 g, 49.44 mmol, 3 eqiv) at 0° C. Resultant reaction mixture was stirred for 4 h at RT. Reaction was monitored by TLC and LCMS. After completion of reaction, water (25 mL) was added to the reaction mixture and extracted with ethyl acetate (50 mL×2). Combined all organic layers and washed with water (50 mL×3) and brine solution (50 mL). The organic layer was dried over anhydrous sodium sulphate and concentrated under reduced pressure to obtain desired product LCMS: 267 [M+H]⁺

Step—4: Synthesis of 3-((dimethylamino)methyl)-7-nitro-3,4-dihydroisoquinolin-1(2H)-one: To a solution of 3-nitro-N-(pivaloyloxy)benzamide (1 g, 3.75 mmol, 1 eqiv) in MeOH (10 mL) was added Bis[(pentamethylcyclopentadienyl)dichloro-rhodium] (120 mg. 0.180 mmol, 0.05 equiv) followed by N,N-dimethylallylamine (380 mg, 4.5 mmol, 1.2 eqiv). Resultant mass was stirred under microwave at 60° C. for 1 h. Reaction was monitored by TLC and LCMS. Reaction mass was diluted with ethyl acetate (40 mL) was washed with saturated solution of sodium carbonate. Collected the organic layer and dried over anhydrous sodium sulphate and concentrated under reduced pressure and purified the compound by making HCl salt of compound to afford desired product. LCMS: 250 [M+H]⁺

Step—5: Synthesis of 7-amino-3-((dimethylamino)methyl)-3,4-dihydroisoquinolin-1(2H)-one: To a stirred solution of 3-((dimethylamino)methyl)-7-nitro-3,4-dihydroisoquinolin-1(2H)-one (400 mg, 1.25 mmol, 1 eqiv) in EtOH (10 mL), was added Iron (675 mg, 12.5 mmol, 10 eqiv), ammonium chloride (700 mg, 12.5 mmol, 10 eqiv) and water (3 ml). The resultant reaction mixture was heated at 80° C. for 2 h. Reaction was monitored by TLC and LCMS. Reaction mixture was filtered through the celite. The filtrate was concentrated under reduced pressure. Aqueous layer was extracted by the ethyl acetate (50 mL). Aqueous layer was concentrated under reduced pressure and 10% solution of methanol in DCM (30 mL) was added and sonicated. Filtered the suspension and filtrate was concentrated to obtain desired product. LCMS: 220 [M+H]⁺

Step—6: Synthesis of 8-cyclopentyl-2-((3-((dimethylamino)methyl)-1-oxo-1,2,3,4-tetrahydroisoquinolin-7-yl)amino)-7-oxo-7,8-dihydropyrido[2,3-d]pyrimidine-6-carbonitrile: To a suspension of 8-cyclopentyl-2-(methylsulfinyl)-7-oxo-7,8-dihydropyrido[2,3-d]pyrimidine-6-carbonitrile (100 mg, 0.33 mmol, 1 eqiv) in toluene (3 mL), was 7-amino-3-((dimethylamino)methyl)-3,4-dihydroisoquinolin-1(2H)-one (68 mg, 0.363 mmol, 1.1 eqiv). Resultant reaction mixture was stirred at 110° C. for 16 h. Reaction was monitored by LCMS. After completion of reaction, reaction mass was concentrated under reduced pressure to afford crude compound. Crude compound was purified by reverse phase HPLC to afford. LCMS: 458 [M+H]⁺; ¹H NMR: (400 MHz, Methanol-d4) δ 8.78 (s, 1H), 8.46 (s, 1H), 8.38 (s, 1H), 7.67 (s, 1H), 7.32 (d, J=8.2 Hz, 1H), 6.05 (q, J=8.8 Hz, 1H), 3.87 (tt, J=9.6, 5.0 Hz, 1H), 3.35 (s, 2H), 3.04 (dd, J=15.8, 4.7 Hz, 1H), 2.84 (dd, J=15.7, 9.6 Hz, 1H), 2.56 (dd, J=12.3, 8.7 Hz, 1H), 2.45 (dd, J=12.3, 5.4 Hz, 1H), 2.32 (s, 6H), 2.24 (s, 2H), 1.95 (s, 2H), 1.70 (t, J=6.6 Hz, 2H).

### Example—S8: Synthesis of 8-cyclopentyl-7-oxo-2-((3-oxoisoindolin-5-yl)amino)-7,8-dihydropyrido[2,3-d]pyrimidine-6-carbonitrile (Compound No. 24)

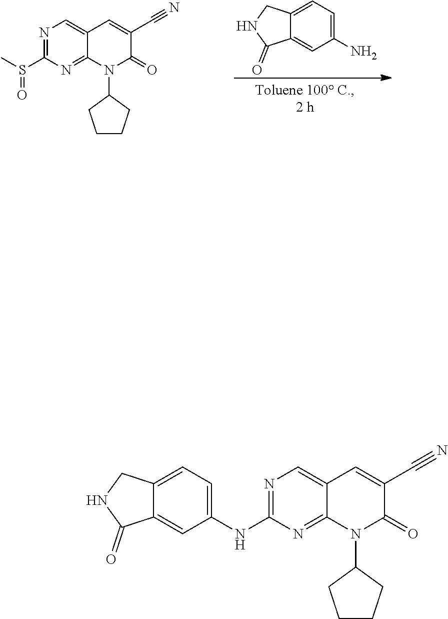 [SMILES: CS(=O)c1ncc2cc(C#N)c(=O)n(C3CCCC3)c2n1 ; Nc1ccc2c(c1)C(=O)NC2 ; N#Cc1cc2cnc(Nc3ccc4c(c3)C(=O)NC4)nc2n(C2CCCC2)c1=O]

To a suspension of 8-cyclopentyl-2-(methylsulfinyl)-7-oxo-7,8-dihydropyrido[2,3-d]pyrimidine-6-carbonitrile (50 mg, 0.16 mmol, 1 eqiv) in toluene (2 mL), was added 6-aminoisoindolin-1-one (26.8 mg, 0.18 mmol, 1.1 eqiv). Resultant reaction mixture was stirred at 100° C. for 2 h. Reaction was monitored by LCMS. After completion of reaction, reaction mass concentrated under reduced pressure. Product was triturated from methanol to afford desired product. LCMS: 387 [M+H]⁺; ¹H NMR: (400 MHz, DMSO-d₆) δ 10.72 (s, 1H), 8.88 (s, 1H), 8.57 (d, J=18.7 Hz, 2H), 8.22 (s, 1H), 7.81-7.74 (m, 1H), 7.56 (d, J=8.2 Hz, 1H), 5.87 (s, 1H), 4.35 (s, 2H), 3.00 (s, 2H), 2.17 (q, J=8.7 Hz, 2H), 1.93-1.77 (m, 2H), 1.67-1.59 (m, 2H).

### Example—S9: Synthesis of 8-cyclopentyl-7-oxo-2-((1,2,3,4-tetrahydroisoquinolin-7-yl)amino)-7,8-dihydropyrido[2,3-d]pyrimidine-6-carbonitrile (Compound No. 29)

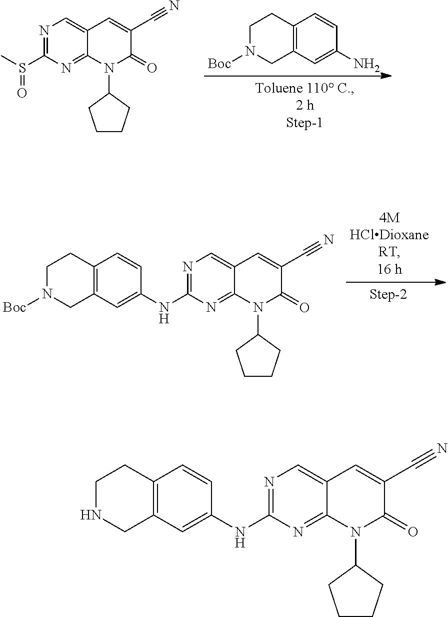 [SMILES: CS(=O)c1ncc2cc(C#N)c(=O)n(C3CCCC3)c2n1 ; CC(C)(C)OC(=O)N1CCc2ccc(N)cc2C1 ; CC(C)(C)OC(=O)N1CCc2ccc(Nc3ncc4cc(C#N)c(=O)n(C5CCCC5)c4n3)cc2C1 ; N#Cc1cc2cnc(Nc3ccc4c(c3)CNCC4)nc2n(C2CCCC2)c1=O]

Step—1: Synthesis of tert-butyl 7-((6-cyano-8-cyclopentyl-7-oxo-7,8-dihydropyrido[2,3-d]pyrimidin-2-yl)amino)-3,4-dihydroisoquinoline-2(1H)-carboxylate: To a suspension of 8-cyclopentyl-2-(methylsulfinyl)-7-oxo-7,8-dihydropyrido[2,3-d]pyrimidine-6-carbonitrile (75 mg, 0.248 mmol, 1 eqiv) in toluene (3 mL), was added tert-butyl 7-amino-3,4-dihydro-1H-isoquinoline-2-carboxylate (67.7 mg, 0.273 mmol, 1.1 eqiv). Resultant reaction mixture was stirred at 110° C. for 2 h. Reaction was monitored by LCMS. After completion of reaction, reaction mass concentrated under reduced pressure. Product was triturated from methanol to afford 58 desired product. LCMS: 487.3 [M+H]⁺

Step—2: Synthesis of 8-cyclopentyl-7-oxo-2-((1,2,3,4-tetrahydroisoquinolin-7-yl)amino)-7,8-dihydropyrido[2,3-d]pyrimidine-6-carbonitrile: To a solution of tert-butyl 7-[(6-cyano-8-cyclopentyl-7-oxo-pyrido[2,3-d]pyrimidin-2-yl)amino]-3,4-dihydro-1H-isoquinoline-2-carboxylate (40 mg, 0.1 mmol, 1 eqiv) in dioxane (1 mL) was added to the 4M HCl in dioxane (2 mL). The resultant reaction mixture was stirred at RT for 16 h. Reaction was monitored by LCMS. After completion of reaction, concentrated the reaction mass under reduced pressure to afford desired product. LCMS: 387.3 [M+H]⁺; ¹H NMR: (400 MHz, DMSO-d₆) δ 10.64-10.53 (m, 1H), 9.20 (s, 1H), 8.86 (s, 1H), 8.60 (s, 1H), 7.65 (s, 1H), 7.52 (d, J=8.5 Hz, 1H), 7.24 (d, J=8.3 Hz, 1H), 5.83 (dd, J=14.8, 8.1 Hz, 1H), 4.26 (t, J=4.5 Hz, 2H), 3.42-3.35 (m, 2H), 2.99 (d, J=6.5 Hz, 2H), 2.19 (q, J=9.0, 7.3 Hz, 2H), 1.95 (s, 2H), 1.82 (q, J=8.7, 6.9 Hz, 2H), 1.66-1.58 (m, 2H).

### Example—S10: Synthesis of 8-cyclopentyl-7-oxo-2-((1,2,3,4-tetrahydroisoquinolin-6-yl)amino)-7,8-dihydropyrido[2,3-d]pyrimidine-6-carbonitrile (Compound No. 31)

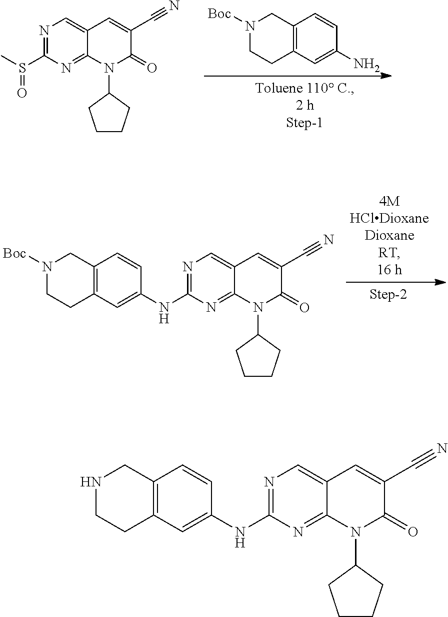 [SMILES: CS(=O)c1ncc2cc(C#N)c(=O)n(C3CCCC3)c2n1 ; CC(C)(C)OC(=O)N1CCc2cc(N)ccc2C1 ; CC(C)(C)OC(=O)N1CCc2cc(Nc3ncc4cc(C#N)c(=O)n(C5CCCC5)c4n3)ccc2C1 ; N#Cc1cc2cnc(Nc3ccc4c(c3)CCNC4)nc2n(C2CCCC2)c1=O]

Step—1: Synthesis of tert-butyl 6-((6-cyano-8-cyclopentyl-7-oxo-7,8-dihydropyrido[2,3-d]pyrimidin-2-yl)amino)-3,4-dihydroisoquinoline-2(1H)-carboxylate: To a suspension of 8-cyclopentyl-2-(methylsulfinyl)-7-oxo-7,8-dihydropyrido[2,3-d]pyrimidine-6-carbonitrile (75 mg, 0.248 mmol, 1 eqiv) in toluene (3 mL), was added tert-butyl 6-amino-3,4-dihydro-1H-isoquinoline-2-carboxylate (67.7 mg, 0.273 mmol, 1.1 eqiv). Resultant reaction mixture was stirred at 110° C. for 2 h. Reaction was monitored by LCMS. After completion of reaction, reaction mass concentrated under reduced pressure. Product was triturated from methanol to afford 70 mg of tert-butyl 6-((6-cyano-8-cyclopentyl-7-oxo-7,8-dihydropyrido[2,3-d]pyrimidin-2-yl)amino)-3,4-dihydroisoquinoline-2(1H)-carboxylate. LCMS: 487.3 [M+H]⁺

Step—2: Synthesis of 8-cyclopentyl-7-oxo-2-((1,2,3,4-tetrahydroisoquinolin-6-yl)amino)-7,8-dihydropyrido[2,3-d]pyrimidine-6-carbonitrile: To a solution of tert-butyl 6-[(6-cyano-8-cyclopentyl-7-oxo-pyrido[2,3-d]pyrimidin-2-yl)amino]-3,4-dihydro-1H-isoquinoline-2-carboxylate. (70 mg, 0.143 mmol, 1 eqiv) in dioxane (2 mL) was added to the 4M HCl in dioxane (2 mL). The resultant reaction mixture was stirred at RT for 16 h. Reaction was monitored by LCMS. After completion of reaction, reaction mass concentrated under reduced pressure to afford desired product. LCMS: 387.3 [M+H]⁺; ¹H NMR: (400 MHz, DMSO-d₆) δ 10.58 (s, 1H), 9.38 (s, 2H), 8.86 (s, 1H), 8.60 (s, 1H), 7.69 (s, 1H), 7.53-7.46 (m, 1H), 7.22 (d, J=8.4 Hz, 1H), 5.82 (d, J=9.0 Hz, 1H), 4.22 (d, J=5.0 Hz, 2H), 3.42-3.32 (m, 2H), 3.00 (t, J=6.4 Hz, 2H), 2.21 (dq, J=15.3, 7.7 Hz, 2H), 1.95 (s, 2H), 1.81 (dq, J=11.7, 6.7, 4.2 Hz, 2H), 1.60 (h, J=7.4 Hz, 2H).

### Example—S1: Synthesis of 8-cyclopentyl-2-((2-methyl-1,2,3,4-tetrahydroisoquinolin-7-yl)amino)-7-oxo-7,8-dihydropyrido[2,3-d]pyrimidine-6-carbonitrile (Compound No. 34)

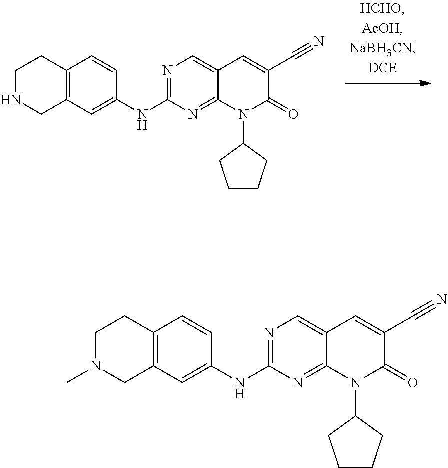 [SMILES: N#Cc1cc2cnc(Nc3ccc4c(c3)CNCC4)nc2n(C2CCCC2)c1=O ; CN1CCc2ccc(Nc3ncc4cc(C#N)c(=O)n(C5CCCC5)c4n3)cc2C1]

To a stirred solution of 8-cyclopentyl-7-oxo-2-(1,2,3,4-tetrahydroisoquinolin-7-ylamino)pyrido[2,3-d]pyrimidine-6-carbonitrile (40 mg, 0.11 mmol, 1 eqiv) in DCE (3 ml) was added HCHO (37%) (9.9 mg, 0.33 mmol, 3 eqiv), glacial acetic acid (33 mg, 0.55 mmoles, 5 eqiv) at 0° C. The reaction mixture was stirred at RT for 1 h, added the sodium cyanoborohydride (21 mg, 0.33 mmol, e eqiv) at 0° C. The reaction mixture allowed to stirrer at RT for 2 h. Reaction progress was monitored by LCMS. After complete consumption of starting material, saturated solution of sodium-bi-carbonate (5 ml) was added. Extracted the reaction mixture with DCM (3×15 ml). Combined all organic phase and washed with brine (20 ml). Dried the organic phase by passing through sodium sulphate, filtered and concentrated under reduced pressure. Purified the crude product by reverse phase HPLC to yield desired product. LCMS: 401.3 [M+H]⁺; ¹H NMR: (400 MHz, Methanol-d₄) δ 8.75 (s, 1H), 8.52 (s, 1H), 8.36 (s, 1H), 7.52 (s, 1H), 7.47-7.40 (m, 1H), 7.18 (d, J=8.3 Hz, 1H), 5.98 (s, 1H), 3.84-3.79 (m, 2H), 3.03-2.95 (m, 4H), 2.61 (s, 3H), 2.29 (dq, J=15.2, 7.9 Hz, 2H), 2.02 (s, 3H), 1.99-1.85 (m, 5H), 1.67 (dd, J=10.5, 5.6 Hz, 2H), 1.37 (s, 2H), 1.29 (s, 1H), 0.89 (s, 1H).

### Example—S12: Synthesis of 8-cyclopentyl-2-((2-methyl-1,2,3,4-tetrahydroisoquinolin-6-yl)amino)-7-oxo-7,8-dihydropyrido[2,3-d]pyrimidine-6-carbonitrile (Compound No. 38)

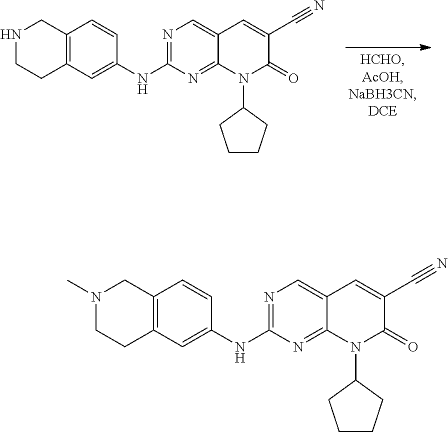 [SMILES: N#Cc1cc2cnc(Nc3ccc4c(c3)CCNC4)nc2n(C2CCCC2)c1=O ; CN1CCc2cc(Nc3ncc4cc(C#N)c(=O)n(C5CCCC5)c4n3)ccc2C1]

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (40 mg, 0.11 mmol, 1 eqiv) in DCE (3 ml) was added formaldehyde solution (37%) (9.9 mg, 0.33 mmol, 3 eqiv), glacial acetic acid (33 mg, 0.55 mmoles, 5 eqiv) at 0° C. The reaction mixture was stirred at RT for 1 h, added the sodium cyanoborohydride (21 mg, 0.33 mmol, e eqiv) at 0° C. The reaction mixture allowed to stirrer at RT for 2 h. Reaction progress was monitored by LCMS. After complete consumption of starting material, saturated solution of sodium-bi-carbonate (5 ml) was added. Extracted the reaction mixture with DCM (3×15 mL). Combined all organic phase and washed with brine (20 mL). Dried the organic phase by passing through sodium sulphate, filtered and concentrated under reduced pressure. Crude product was purified by reverse phase HPLC to afford desired product. LCMS: 401.3 [M+H]⁺; ¹H NMR: (400 MHz, Methanol-d4) δ 8.75 (s, 1H), 8.36 (s, 1H), 7.59 (s, 1H), 7.43-7.35 (m, 1H), 7.10 (d, J=8.3 Hz, 1H), 6.03-5.94 (m, 1H), 3.74 (s, 2H), 3.01 (t, J=6.0 Hz, 2H), 2.90 (t, J=6.1 Hz, 2H), 2.56 (s, 2H), 2.29 (dq, J=14.4, 7.6 Hz, 2H), 2.01 (s, 2H), 1.97-1.82 (m, 2H), 1.73-1.56 (m, 2H)

### Example—S13: Synthesis of 8-cyclopentyl-2-((2-isopropyl-1,2,3,4-tetrahydroisoquinolin-6-yl)amino)-7-oxo-7,8-dihydropyrido[2,3-d]pyrimidine-6-carbonitrile (Compound No. 45)

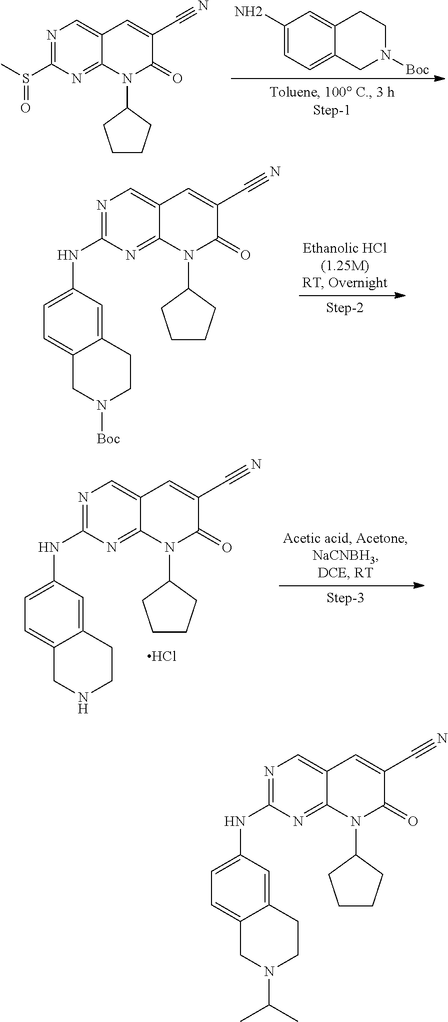

Step—1: Synthesis of tert-butyl 6-((6-cyano-8-cyclopentyl-7-oxo-7,8-dihydropyrido[2,3-d]pyrimidin-2-yl)amino)-3,4-dihydroisoquinoline-2(1H)-carboxylate: To a stirred solution of 8-cyclopentyl-2-(methylsulfinyl)-7-oxo-7, 8-dihydropyrido [2, 3-d]pyrimidine-6-carbonitrile (200 mg, 0.66 mmol, 1 equiv) in toluene (5 mL), was added tert-butyl 6-amino-3,4-dihydroisoquinoline-2(1H)-carboxylate (181 mg, 0.72 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain desired product. LCMS: 487 [M+H]⁺

Step—2: Synthesis of 8-cyclopentyl-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-cyclopentyl-7-oxo-7, 8-dihydropyrido [2, 3-d]pyrimidin-2-yl) amino)-3, 4-dihydroisoquinoline-2(1H)-carboxylate (100 mg, 0.2 mmol, 1 equiv) in 1.25 M HCl in ethanol (5 mL) was allowed to stir for overnight at RT. Progress of the reaction was monitored by LCMS. After completion of the reaction, solvent was removed under reduced pressure to obtain desired product. LCMS: 387.4 [M+H]⁺

Step—3: Synthesis of 8-cyclopentyl-2-((2-isopropyl-1,2,3,4-tetrahydroisoquinolin-6-yl)amino)-7-oxo-7,8-dihydropyrido[2,3-d]pyrimidine-6-carbonitrile: To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (85 mg, 0.22 mmol, 1 equiv) in DCE (5 mL), was added acetone (0.04 mL, 0.66 mmol, 3 equiv), acetic acid (0.06 mL, 1.1 mmol, 5 equiv). The reaction mixture was allowed to stir at room temperature for 1 h. The reaction mixture was cooled to 0° C. NaCNBH₃ (42 mg, 0.66 mmol, 3 equiv) was added to above mixture and reaction mass stirred at room temperature. The reaction mixture was allowed to stir at RT for 1 h. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 429.4 [M+H]⁺; ¹HNMR: (400 MHz, DMSO-d₆) δ 10.55 (d, J=29.1 Hz, 1H), 8.86-8.81 (m, 1H), 8.59-8.54 (m, 1H), 7.63 (d, J=13.7 Hz, 1H), 7.38 (s, 1H), 7.11 (s, 1H), 5.83 (s, 1H), 3.20-3.13 (m, 1H), 3.02 (s, 1H), 2.90-2.82 (m, 2H), 2.19 (dt, J=14.4, 8.0 Hz, 2H), 2.08 (t, J=3.3 Hz, 1H), 1.92 (t, J=8.9 Hz, 2H), 1.81 (s, 2H), 1.59 (s, 2H), 1.23 (s, 2H), 1.13 (s, 6H).

### Example—S14: Synthesis of 8-cyclopentyl-2-((2-(dimethylamino)-2,3-dihydro-1H-inden-5-yl)amino)-7-oxo-7,8-dihydropyrido[2,3-d]pyrimidine-6-carbonitrile (Compound No. 47)

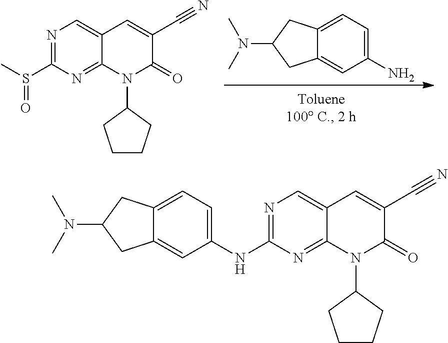

To a suspension of 8-cyclopentyl-2-(methylsulfinyl)-7-oxo-7,8-dihydropyrido[2,3-d]pyrimidine-6-carbonitrile (50 mg, 0.16 mmol, 1 eqiv) in toluene (2 mL), was added N2,N2-dimethyl-2,3-dihydro-1H-indene-2,5-diamine (26.8 mg, 0.18 mmol, 1.1 eqiv). Resultant reaction mixture was stirred at 100° C. for 2 h. Reaction was monitored by LCMS. After completion of reaction, reaction mass concentrated under reduced pressure. Product was triturated from methanol to afford desired product. LCMS: 415 [M+H]⁺; ¹HNMR: (400 MHz, DMSO-d₆) δ 10.52-10.45 (m, 1H), 8.82 (s, 1H), 8.56 (s, 1H), 7.59 (s, 1H), 7.37 (t, J=4.9 Hz, 1H), 7.18 (d, J=8.2 Hz, 1H), 5.85 (s, 1H), 5.77 (s, 1H), 3.20-3.11 (m, 2H), 3.01 (dd, J=15.5, 6.9 Hz, 2H), 2.79 (dq, J=13.8, 8.5 Hz, 2H), 2.23 (d, J=24.1 Hz, 6H), 1.84 (dd, J=44.0, 14.7 Hz, 4H), 1.57 (t, J=7.7 Hz, 2H).

### Example—S15: Synthesis of 8-cyclopentyl-2-((7-(dimethylamino)-5,6,7,8-tetrahydronaphthalen-2-yl)amino)-7-oxo-7,8-dihydropyrido[2,3-d]pyrimidine-6-carbonitrile (Compound No. 48)

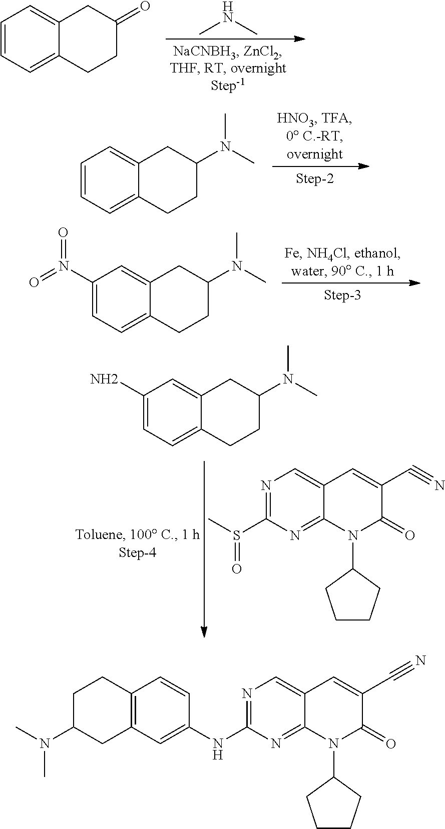

Step—1: Synthesis of N,N-dimethyl-1,2,3,4-tetrahydronaphthalen-2-amine: To a stirred solution of 3, 4-dihydronaphthalen-2(1H)-one (5000 mg, 34.2 mmol, 1 equiv) in THF (50 mL), was added NaCNBH₃ (2155 mg, 34.2 mmol, 1 equiv), ZnCl₂ (2326 mg, 17.1 mmol, 0.5 equiv) and dimethyl amine (2M in TFH) (17 mL, 34.2 mmol, 1 equiv). The resultant reaction mixture was allowed to stir at RT for overnight. Progress of the reaction was monitored by LCMS. After completion of the reaction, solvent was removed under reduced pressure and 1N HCl (100 mL) was added to above residue. The acidic solution was washed with ethyl acetate (100 mL×2), then made alkaline with aq. 5M NaOH solution (50 mL) and extracted with EtoAC (100 mL×3). Organic layer was dried over anhydrous sodium sulphate and concentrated under reduced pressure to obtain desired product. LCMS: 176 [M+H]⁺

Step—2: Synthesis of N,N-dimethyl-7-nitro-1,2,3,4-tetrahydronaphthalen-2-amine: To a stirred solution of N, N-dimethyl-1, 2, 3, 4-tetrahydronaphthalen-2-amine (950 mg, 5.4 mmol, 1 equiv) in THF (5 mL), was added HNO3 (0.9 mL, 21.7 mmol, 4 equiv) at 0° C. The resultant reaction mixture was allowed to stir at RT for overnight. Progress of the reaction was monitored by LCMS. After completion of the reaction, the mixture was diluted with ice water (20 mL), then made alkaline with aq. 5M NaOH solution (10 mL) and extracted with EtoAC (100 mL×2). Organic layer was dried over anhydrous sodium sulphate and concentrated under reduced pressure to obtain desired product. LCMS: 221 [M+H]⁺

Step—3: Synthesis of N2,N2-dimethyl-1,2,3,4-tetrahydronaphthalene-2,7-diamine: To a stirred solution of N, N-dimethyl-7-nitro-1, 2, 3, 4-tetrahydronaphthalen-2-amine (500 mg, 1.87 mmol, 1 equiv) in ethanol (9 mL), water (3 mL), was added iron powder (535 mg, 9.5 mmol, 3 equiv) and ammonium chloride (335 mg, 6.2 mmol, 2 equiv). The resultant reaction mixture was allowed to stir at 90° C. for 1 h. Progress of the reaction was monitored by TLC and LCMS. After completion of the reaction, the reaction mixture was passes through celite bed and the filtrate was concentrated under reduced pressure to obtain desired product. LCMS: 191 [M+H]⁺

Step—4: Synthesis of 8-cyclopentyl-2-((7-(dimethylamino)-5,6,7,8-tetrahydronaphthalen-2-yl)amino)-7-oxo-7,8-dihydropyrido[2,3-d]pyrimidine-6-carbonitrile: To a stirred solution of 8-cyclopentyl-2-(methylsulfinyl)-7-oxo-7, 8-dihydropyrido [2, 3-d]pyrimidine-6-carbonitrile (150 mg, 0.49 mmol, 1 equiv) in toluene (5 mL), was added a mixture of N2,N2-dimethyl-1,2,3,4-tetrahydronaphthalene-2,7-diamine and N2,N2-dimethyl-1,2,3,4-tetrahydronaphthalene-2,6-diamine (104 mg, 0.54 mmol, 1.1 equiv). The resultant reaction mixture was stir at 100° C. for 1 h. Progress of the reaction was monitored by LCMS. After completion of the reaction, solvent was removed under reduced pressure to obtain crude which was purified by reverse phase HPLC to obtain desired product. LCMS: 429 [M+H]⁺; ¹HNMR: (400 MHz, DMSO-d₆) δ 10.54 (s, 1H), 8.84 (s, 1H), 8.57 (s, 1H), 7.57 (s, 1H), 7.45 (d, J=8.2 Hz, 1H), 7.25 (d, J=6.5 Hz, 1H), 5.83 (s, 1H), 3.59 (d, J=11.8 Hz, 1H), 3.09-2.93 (m, 2H), 2.84 (s, 6H), 2.21 (q, J=8.9, 7.5 Hz, 4H), 1.93 (s, 2H), 1.85-1.72 (m, 4H), 1.60 (q, J=6.2, 5.7 Hz, 2H).

### Example—S16: Synthesis of 8-cyclopentyl-2-((5-methyl-4,5,6,7-tetrahydropyrazolo[1,5-a]pyrazin-2-yl)amino)-7-oxo-7,8-dihydropyrido[2,3-d]pyrimidine-6-carbonitrile (Compound No. 52)

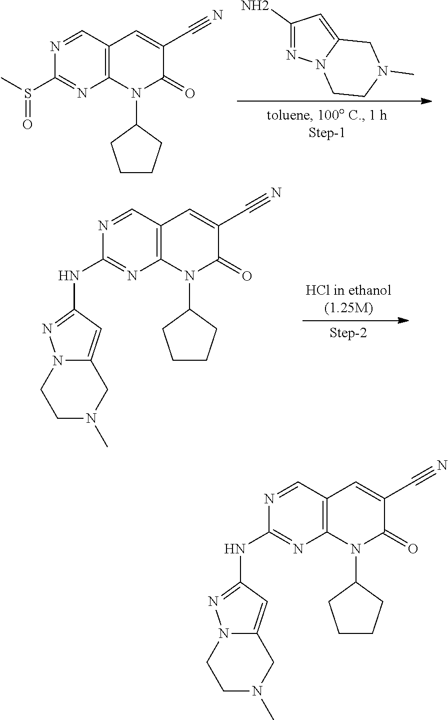

Step—1: Synthesis of 8-cyclopentyl-2-((5-methyl-4,5,6,7-tetrahydropyrazolo[1,5-a]pyrazin-2-yl)amino)-7-oxo-7,8-dihydropyrido[2,3-d]pyrimidine-6-carbonitrile: To a stirred solution of 8-cyclopentyl-2-(methylsulfonyl)-7-oxo-7,8-dihydropyrido[2,3-d]pyrimidine-6-carbonitrile (100 mg, 0.33 mmol, 1 equiv) in toluene (5 mL), was added 5-methyl-4,5,6,7-tetrahydropyrazolo[1,5-a]pyrazin-2-amine (55 mg, 0.36 mmol, 1.1 equiv). The resultant reaction mixture was stir at 100° C. for 1 h. Progress of the reaction was monitored by LCMS. After completion of the reaction, solvent was removed under reduced pressure to obtain crude which was purified by recrystallization with methanol to obtain desired product. LCMS: 391 [M+H]⁺

Step—2: Synthesis of 8-cyclopentyl-2-((5-methyl-4,5,6,7-tetrahydropyrazolo[1,5-a]pyrazin-2-yl)amino)-7-oxo-7,8-dihydropyrido[2,3-d]pyrimidine-6-carbonitrile: tert-butyl 4-(4-((6-cyano-8-cyclopentyl-7-oxo-7, 8-dihydropyrido [2, 3-d]pyrimidin-2-yl) amino)-1H-pyrazol-1-yl) piperidine-1-carboxylate (10 mg, 0.02 mmol, 1 equiv) was taken in 1.25 M HCl in ethanol (5 mL) and the resultant reaction mixture was allowed to stir at RT for 1 h. Solvent was removed under reduced pressure and the residue was dried under lyophilizer to obtain desired product. LCMS: 391 [M+H]⁺; ¹HNMR: (400 MHz, DMSO-d₆) δ 10.90 (d, J=43.6 Hz, 1H), 8.84 (s, 1H), 8.59 (s, 1H), 6.51 (s, 1H), 5.83 (s, 1H), 4.69 (d, J=14.5 Hz, 1H), 4.36 (d, J=5.8 Hz, 3H), 3.87 (d, J=13.4 Hz, 1H), 3.70 (s, 1H), 2.96 (s, 3H), 2.21 (dq, J=14.9, 7.9 Hz, 2H), 1.96 (s, 2H), 1.86-1.74 (m, 2H), 1.65-1.59 (m, 2H).

### Example—S17: Synthesis of 8-cyclopentyl-2-((2-(dimethylglycyl)-1,2,3,4-tetrahydroisoquinolin-7-yl)amino)-7-oxo-7,8-dihydropyrido[2,3-d]pyrimidine-6-carbonitrile (Compound No. 53)

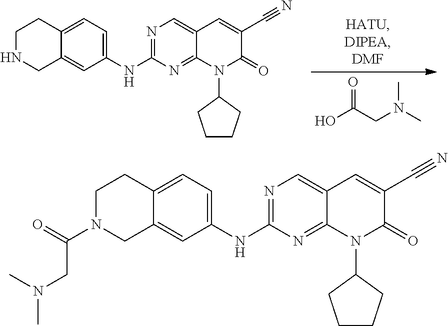

To a stirred solution of 8-cyclopentyl-7-oxo-2-((1,2,3,4-tetrahydroisoquinolin-7-yl)amino)-7,8-dihydropyrido[2,3-d]pyrimidine-6-carbonitrile (20 mg, 0.05 mmol, 1 equiv) in DMF (2 mL), was added dimethylglycine (7.3 mg, 0.07 mmol, 1.5 equiv) followed by addition of HATU (27 mg, 0.07 mmol, 1.5 equiv). The resultant reaction mixture was allowed to stir at room temperature for 2 h. Progress of the reaction was monitored by LCMS. After completion of the reaction, the reaction mixture was diluted with water (25 mL). Product precipitates out was filtered and dried to afford desired product. LCMS: 472 [M+H]⁺; ¹HNMR: (400 MHz, DMSO-d₆) δ 8.83 (s, 1H), 8.53 (s, 1H), 7.64 (s, 1H), 7.39 (t, J=11.0 Hz, 1H), 7.17 (d, J=8.4 Hz, 1H), 5.80 (s, 1H), 4.71 (s, 1H), 4.59 (s, 1H), 3.70 (t, J=5.9 Hz, 1H), 3.34 (d, J=18.2 Hz, 2H), 2.83 (t, J=5.9 Hz, 1H), 2.74 (t, J=6.4 Hz, 1H), 2.28 (d, J=14.5 Hz, 5H), 2.17 (d, J=10.5 Hz, 2H), 1.89 (s, 2H), 1.85-1.75 (m, 2H), 1.64-1.56 (m, 2H).

### Example—S18: Synthesis of 8-cyclopentyl-2-((2-(dimethylglycyl)-1,2,3,4-tetrahydroisoquinolin-6-yl)amino)-7-oxo-7,8-dihydropyrido[2,3-d]pyrimidine-6-carbonitrile (Compound No. 54)

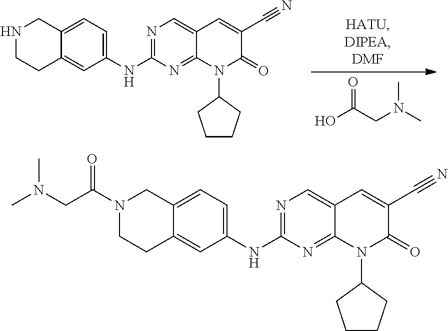

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (20 mg, 0.05 mmol, 1 equiv) in DMF (2 mL), was added dimethylglycine (7.3 mg, 0.07 mmol, 1.5 equiv) followed by addition of HATU (27 mg, 0.07 mmol, 1.5 equiv). The resultant reaction mixture was allowed to stir at room temperature for 2 h. Progress of the reaction was monitored by LCMS. After completion of the reaction, the reaction mixture was diluted with water (25 mL). Product precipitates out was filtered and dried to afford: LCMS: 472 [M+H]⁺; ¹HNMR: (400 MHz, DMSO-d₆) δ 8.81 (s, 1H), 8.50 (s, 1H), 7.59 (s, 1H), 7.39 (dt, J=14.4, 7.1 Hz, 1H), 7.21-7.13 (m, 1H), 5.83-5.73 (m, 1H), 4.67 (s, 1H), 4.57 (s, 1H), 3.30 (d, J=13.6 Hz, 2H), 2.86 (d, J=13.0 Hz, 1H), 2.75 (s, 1H), 2.27 (s, 6H), 2.20-2.11 (m, 2H), 1.83 (dq, J=41.9, 12.9, 10.4 Hz, 4H), 1.61-1.52 (m, 2H).

### Example—S19: Synthesis of 8-cyclopentyl-7-oxo-2-((2-oxo-1-(piperidin-4-yl)-1,2-dihydropyridin-4-yl)amino)-7,8-dihydropyrido[2,3-d]pyrimidine-6-carbonitrile (Compound No. 60)

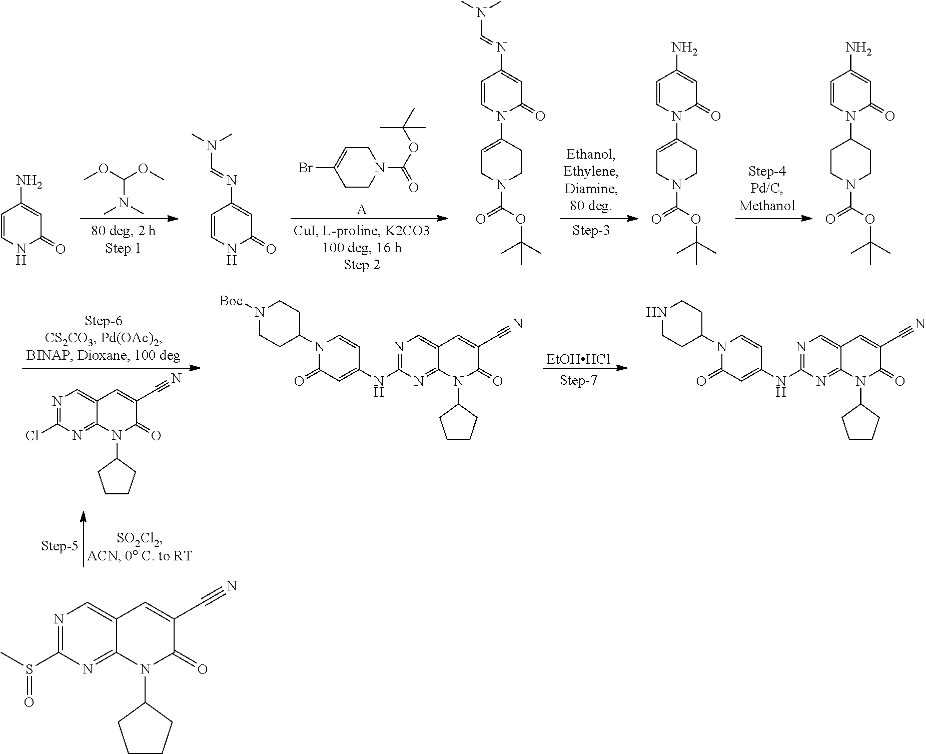

Step—1: Synthesis of (E/Z)-N,N-dimethyl-N′-(2-oxo-1,2-dihydropyridin-4-yl)formimidamide: 4-aminopyridin-2(1H)-one (200 mg, 1.8 mmol, 1 equiv) was taken in 1,1-dimethoxy-N,N-dimethylmethanamine (4 mL) and the resultant reaction mixture was stirred at 80° C. for 2 h. Progress of the reaction was monitored by LCMS. After completion of the reaction, the reaction mixture was concentrated under vacuum to afford desired product. LCMS: 166 [M+H]⁺

Step—2: Synthesis of tert-butyl (E/Z)-4-(((dimethylamino)methylene)amino)-2-oxo-3′,6′-dihydro-2H-[1,4′-bipyridine]-1′(2′H)-carboxylate: To a solution of (E/Z)-N,N-dimethyl-N′-(2-oxo-1,2-dihydropyridin-4-yl)formimidamide (500 mg, 3 mmol, 1 equiv) in DMF (10 mL), was added tert-butyl 4-bromo-3,6-dihydropyridine-1(2H)-carboxylate (730 mg, 4.5 mmol, 1.5 equiv) followed by addition of potassium carbonate (700 mg, 5.1 mmol, 1.7 equiv), CuI (57 mg, 0.3 mmol, 0.1 equiv), L-proline (68 mg, 0.6 mmol, 0.2 equiv). The resultant reaction mixture was stirred at 100° C. for 16 h. Progress of the reaction was monitored by TLC and LCMS. After completion of the reaction, the reaction mixture was diluted with water (30 mL) and extracted with EtOAc (50 mL). The organic layer dried over anhydrous sodium sulphate and concentrated under reduced pressure to obtain crude compound, which was purified by making HCL salt to afford desired product. LCMS: 347 [M+H]⁺

Step—3: Synthesis of tert-butyl 4-amino-2-oxo-3′,6′-dihydro-2H-[1,4′-bipyridine]-1′(2′H)-carboxylate: To a solution of tert-butyl (E/Z)-4-(((dimethylamino)methylene)amino)-2-oxo-3′,6′-dihydro-2H-[1,4′-bipyridine]-1′(2′H)-carboxylate (135 mg, 0.4 mmol, 1 equiv) in ethanol (5 mL), was added ethylene diamine (35 mg, 0.6 mmol, 1.5 equiv. The resultant reaction mixture was stirred at 80° C. for 3 h. Progress of the reaction was monitored by TLC and LCMS. After completion of the reaction, the reaction mixture was concentrated, diluted with water (10 mL) and extracted with EtOAc (10 mL). The organic layer dried over anhydrous sodium sulphate and concentrated under reduced pressure to obtain desired product. LCMS: 292 [M+H]⁺

Step—4: Synthesis of tert-butyl 4-(4-amino-2-oxopyridin-1(2H)-yl)piperidine-1-carboxylate: To a stirred solution of tert-butyl 4-amino-2-oxo-3′,6′-dihydro-2H-[1,4′-bipyridine]-1′(2′H)-carboxylate (200 mg, 0.68 mmol, 1 equiv) in methanol (5 mL), was added Pd/C (10 wt. %) (50 mg). The resultant reaction mixture was allowed to stir at RT for by purging hydrogen gas for 6 h. Progress of the reaction was monitored by TLC and LCMS. After completion of the reaction, the mixture was passes through cealite bed and the filtrate was concentrated under reduced pressure to afford desired product. 294 [M+H]⁺

Step—5: Synthesis of 2-chloro-8-cyclopentyl-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (200 mg, 0.66 mmol, 1 equiv) in acetonitrile (5 mL), was added sulfuryl chloride (0.11 ml, 0.66 mmol, 1 equiv) dropwise at 0° C. The resultant reaction mixture was allowed to stir at room temperature for 2 h. Progress of the reaction was monitored by TLC and LCMS. After completion of the reaction, the reaction mixture was concentrated, neutralized by saturated NaHCO₃ solution (10 mL) and extracted with EtOAc (10 mL). The organic layer was dried over anhydrous sodium sulphate and concentrated under reduced pressure to afford desired product. LCMS: 275 [M+H]⁺

Step—6: Synthesis of tert-butyl 4-(4-((6-cyano-8-cyclopentyl-7-oxo-7,8-dihydropyrido[2,3-d]pyrimidin-2-yl)amino)-2-oxopyridin-1(2H)-yl)piperidine-1-carboxylate: To a solution of 2-chloro-8-cyclopentyl-7-oxo-7,8-dihydropyrido[2,3-d]pyrimidine-6-carbonitrile (120 mg, 0.44 mmol, 1 equiv) in dioxane (5 mL), was added obtain tert-butyl 4-(4-amino-2-oxopyridin-1(2H)-yl)piperidine-1-carboxylate (141 mg, 0.48 mmol, 1.1 equiv) and cesium carbonate (214 mg, 0.66 mmol, 1.5 equiv). The reaction mixture was degassed with nitrogen gas for 10 min. followed by the addition of palladium acetate (10 mg, 0.044 mmol, 0.1 equiv) and BINAP (55 mg, 0.08 mmol, 0.2 equiv) again purged nitrogen for 5 min. The resultant reaction mixture was stirred at 100° C. for 16 h. Progress of the reaction was monitored by TLC and LCMS. After completion of the reaction, the reaction mixture was diluted with water (10 mL) and extracted with EtOAc (15 mL). The organic layer dried over anhydrous sodium sulphate and concentrated under reduced pressure to obtain desired product. LCMS: 532 [M+H]⁺

Step—7: Synthesis of 8-cyclopentyl-7-oxo-2-((2-oxo-1-(piperidin-4-yl)-1,2-dihydropyridin-4-yl)amino)-7,8-dihydropyrido[2,3-d]pyrimidine-6-carbonitrile: To a solution of tert-butyl 4-(4-((6-cyano-8-cyclopentyl-7-oxo-7,8-dihydropyrido[2,3-d]pyrimidin-2-yl)amino)-2-oxopyridin-1(2H)-yl)piperidine-1-carboxylate (120 mg, 0.22 mmol, 1 equiv) was taken in 1.25 M HCl in ethanol (3 mL) and the resultant reaction mixture was allowed to stir at 50° C. for 1 h. Progress of the reaction was monitored by LCMS. After completion of the reaction, solvent was removed under reduced pressure and the residue was diluted with water (5 ml) and was extracted with ethyl acetate (7 mL). Aqueous layer was neutralized by saturated NaHCO₃ solution and was extracted with ethyl acetate. Organic layer dried over sodium sulphate evaporated under vacuum to afford desired product. LCMS: 432 [M+H]⁺; ¹HNMR: (400 MHz, Methanol-d4) δ 8.89 (s, 1H), 8.47 (s, 1H), 7.57 (d, J=7.6 Hz, 1H), 7.28 (d, J=2.5 Hz, 1H), 6.84-6.76 (m, 1H), 6.02 (p, J=8.6 Hz, 1H), 4.99-4.89 (m, 2H), 3.53 (d, J=12.5 Hz, 2H), 3.18 (d, J=12.5 Hz, 3H), 2.30 (dq, J=15.5, 8.0 Hz, 3H), 2.20-2.01 (m, 2H), 1.96 (q, J=9.2, 8.5 Hz, 2H), 1.75 (p, J=7.2, 6.6 Hz, 2H).

### Example—S20: Synthesis of 8-cyclopentyl-7-oxo-2-((5,6,7,8-tetrahydro-1,6-naphthyridin-2-yl)amino)-7,8-dihydropyrido[2,3-d]pyrimidine-6-carbonitrile (Compound No. 61)

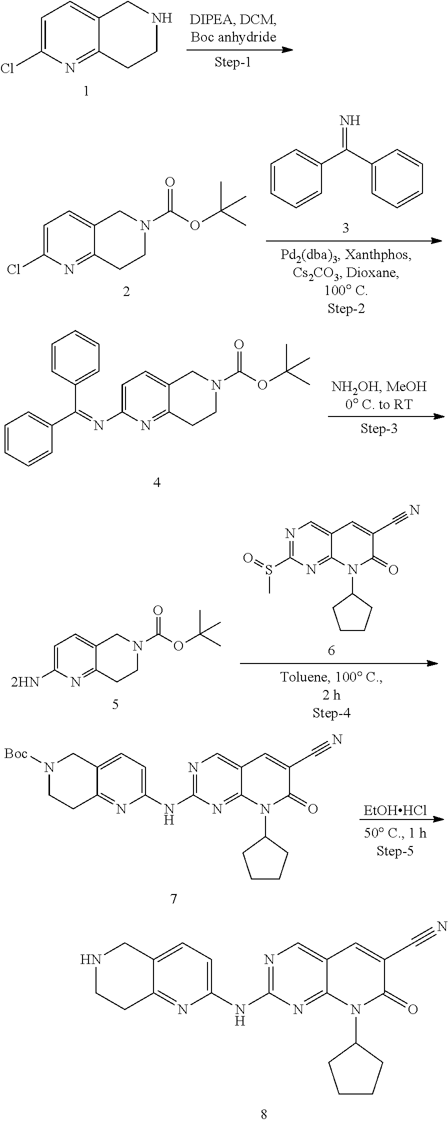

Step—1: Synthesis of tert-butyl 2-chloro-7,8-dihydro-1,6-naphthyridine-6(5H)-carboxylate: To the solution of 2-chloro-5,6,7,8-tetrahydro-1,6-naphthyridine (2000 mg, 11.9 mmol, 1 equiv), taken in DCM (30 mL), was added DIPEA (1.84 g, 14.28 mmol, 1.2 equiv) at 0° C. then was added Boc Anhydride (2.850 mg, 18.08 mmol, 1.1 equiv), resulted reaction mixture was allowed to stir at RT for 2 h. Progress of reaction was monitored by LCMS/TLC. After completion the reaction, mixture was diluted with water and extracted with DCM (200 mL). Organic layer was washed with water (30 mL) and brine solution (40 mL), resulted organic layer was dried over anhydrous sodium sulphate and purified by combi-flash column to afford desired product. LCMS: 269 [M+H]+

Step—2: Synthesis of tert-butyl 2-((diphenylmethylene)amino)-7,8-dihydro-1,6-naphthyridine-6(5H)-carboxylate: To the solution of tert-butyl 2-chloro-7,8-dihydro-1,6-naphthyridine-6(5H)-carboxylate (1 g, 3.73 mmol, 1 equiv), taken in dioxane (20 mL) was added diphenylmethanimine (0.74 g, 4.13 mmol, 1.1 equiv), cesium carbonate (2.42 g, 7.46 mmol, 1.5 equiv). The reaction mixture was degassed with nitrogen gas for 5 min., followed by the addition of Pd₂(dba)₃ (340 mg, 0.37 mmol, 0.1 equiv) and xanthphos (740 mg, 0.74 mmol, 0.2 equiv). The resultant reaction mixture was allowed to stir at 100° C. for 16 h. Progress of the reaction was monitored by TLC and LCMS. After completion of the reaction, diluted with water (50 mL) and extracted with ethyl acetate (200 mL). Organic layer was washed with water (100 mL) and brine solution (50 mL). Organic layer was dried over anhydrous sodium sulphate and concentrated under reduced pressure to obtain crude, which was purified by normal phase combi flash to obtain desired product. LCMS: 414 [M+H]⁺

Step—3: Synthesis of tert-butyl 2-amino-7,8-dihydro-1,6-naphthyridine-6(5H)-carboxylate: To a solution of tert-butyl 2-((diphenylmethylene)amino)-7,8-dihydro-1,6-naphthyridine-6(5H)-carboxylate (1469 mg, 3.5 mmol, 1 equiv) taken in methanol (25 mL), was added hydroxylamine hydrochloride (476 mg, 7 mmol, 2 equiv), resulted reaction mixture allowed to stir at RT for 3 h. Progress of the reaction was monitored by TLC and LCMS. After completion of the reaction, diluted with water (30 mL) and extracted with ethyl acetate (100 mL). Organic layer was washed with water (20 mL) and brine solution (30 mL). Organic layer was dried over anhydrous sodium sulphate and concentrated under reduced pressure to obtain crude, which was purified by normal phase combi flash to obtain desired product. LCMS: 250 [M+H]⁺

Step—4: Synthesis of tert-butyl 2-((6-cyano-8-cyclopentyl-7-oxo-7,8-dihydropyrido[2,3-d]pyrimidin-2-yl)amino)-7,8-dihydro-1,6-naphthyridine-6(5H)-carboxylate: To the solution of tert-butyl 2-amino-7,8-dihydro-1,6-naphthyridine-6(5H)-carboxylate (100 mg, 0.33 mmol, 1 equiv), taken in toluene (5 mL), was added 8-cyclopentyl-2-(methylsulfonyl)-7-oxo-7,8-dihydropyrido[2,3-d]pyrimidine-6-carbonitrile (90 mg, 0.36 mmol, 1.1 equiv), resulted reaction mixture was allowed to stir 100° C. for 4 h. Progress of reaction was monitored by LCMS/TLC. After completion the reaction mixture was diluted with water and extracted with ethylacetate (30 mL). Organic layer was washed with water (20 mL) and brine solution (20 mL), resulted organic layer was dried over anhydrous sodium sulphate and purified by combi-flash column to afford desired product. LCMS: 489 [M+H]⁺

Step—5: Synthesis of 8-cyclopentyl-7-oxo-2-((5,6,7,8-tetrahydro-1,6-naphthyridin-2-yl)amino)-7,8-dihydropyrido[2,3-d]pyrimidine-6-carbonitrile: To a solution of tert-butyl 2-((6-cyano-8-cyclopentyl-7-oxo-7,8-dihydropyrido[2,3-d]pyrimidin-2-yl)amino)-7,8-dihydro-1,6-naphthyridine-6(5H)-carboxylate (52 mg, 0.109 mmol, 1 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure and the residue was dried under lyophilizer to obtain desired product. LCMS: 387 [M+H]⁺; ¹H NMR: (400 MHz, DMSO-d₆) δ 10.82 (s, 1H), 9.41 (s, 2H), 8.91 (s, 1H), 8.64 (s, 1H), 7.92 (d, J=8.6 Hz, 1H), 7.73 (d, J=8.5 Hz, 1H), 5.83 (p, J=8.7 Hz, 1H), 4.28 (d, J=4.8 Hz, 2H), 3.54-3.44 (m, 2H), 3.04 (t, J=6.4 Hz, 2H), 2.23 (dq, J=14.4, 8.4 Hz, 2H), 1.95 (dt, J=12.2, 6.2 Hz, 2H), 1.86-1.74 (m, 2H), 1.59 (dt, J=15.5, 8.0 Hz, 2H).

### Example—S21: Synthesis of 8-cyclopentyl-2-((2-(2-(dimethylamino)ethyl)-1,2,3,4-tetrahydroisoquinolin-7-yl)amino)-7-oxo-7,8-dihydropyrido[2,3-d]pyrimidine-6-carbonitrile (Compound No. 62)

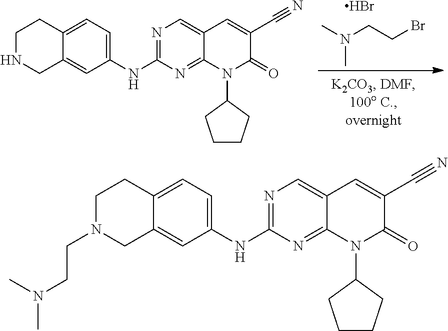

To a stirred solution of 8-cyclopentyl-7-oxo-2-((1,2,3,4-tetrahydroisoquinolin-7-yl)amino)-7,8-dihydropyrido[2,3-d]pyrimidine-6-carbonitrile (100 mg, 0.23 mmol, 1 equiv) in DMF (5 mL), was added K₂CO₃ (65 mg, 0.47 mmol, 2 equiv) and 2-bromo-N,N-dimethylethan-1-amine.HBr (182 mg, 0.47 mmol, 2 equiv). The resultant reaction mixture was allowed to stir at 100° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 458 [M+H]⁺; ¹HNMR: (400 MHz, DMSO-d₆): δ10.48 (s, 1H), 8.83 (s, 1H), 8.56 (s, 1H), 7.57 (br. s., 1H), 7.34 (br. s., 1H), 7.04 (d, J=7.8 Hz, 1H), 5.83 (br. s., 1H), 3.55 (br. s., 4H), 3.17 (br. s., 2H), 2.78 (br. s., 2H), 2.68 (d, J=7.8 Hz, 2H), 2.61 (d, J=7.8 Hz, 2H), 2.21 (s, 6H), 1.89 (br. s., 2H), 1.81 (br. s., 2H), 1.58 (br. s., 2H).

### Example—S22: Synthesis of 8-cyclopentyl-2-((2-(2-(dimethylamino)ethyl)-1,2,3,4-tetrahydroisoquinolin-6-yl)amino)-7-oxo-7,8-dihydropyrido[2,3-d]pyrimidine-6-carbonitrile (Compound No. 63)

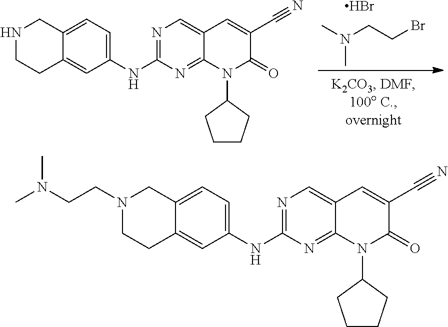

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.23 mmol, 1 equiv) in DMF (5 mL), was added K₂CO₃ (65 mg, 0.47 mmol, 2 equiv) and 2-bromo-N,N-dimethylethan-1-amine.HBr (182 mg, 0.47 mmol, 2 equiv). The resultant reaction mixture was allowed to stir at 100° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 458 [M+H]⁺; ¹HNMR: (400 MHz, DMSO-d₆): δ 10.48 (s, 1H), 8.83 (s, 1H), 8.56 (s, 1H), 7.57 (br. s., 1H), 7.34 (br. s., 1H), 7.04 (d, J=7.8 Hz, 1H), 5.83 (br. s., 1H), 3.55 (br. s., 4H), 3.17 (br. s., 2H), 2.78 (br. s., 2H), 2.68 (d, J=7.8 Hz, 2H), 2.61 (d, J=7.8 Hz, 2H), 2.21 (s, 6H), 1.89 (br. s., 2H), 1.81 (br. s., 2H), 1.58 (br. s., 2H).

### Example—S23: Synthesis of 8-cyclopentyl-2-((6-(2-(dimethylamino)ethyl)-5,6,7,8-tetrahydro-1,6-naphthyridin-2-yl)amino)-7-oxo-7,8-dihydropyrido[2,3-d]pyrimidine-6-carbonitrile (Compound No. 64)

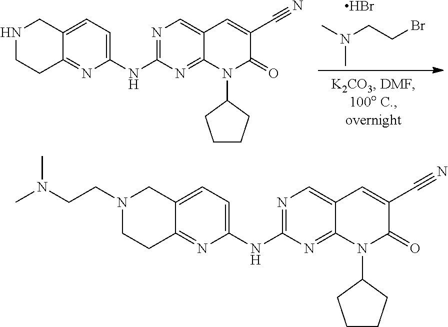

To a stirred solution of 8-cyclopentyl-7-oxo-2-((5,6,7,8-tetrahydro-1,6-naphthyridin-2-yl)amino)-7,8-dihydropyrido[2,3-d]pyrimidine-6-carbonitrile (50 mg, 0.12 mmol, 1 equiv) in DMF (5 mL), was added K₂CO₃ (35 mg, 0.25 mmol, 2 equiv) and 2-bromo-N,N-dimethylethan-1-amine.HBr (60 mg, 0.25 mmol, 2 equiv). The resultant reaction mixture was allowed to stir at 100° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 459 [M+H]⁺; ¹HNMR: (400 MHz, DMSO-d₆): 8.88 (s, 1H), 8.61 (s, 1H), 7.76 (d, J=6.4 Hz, 1H), 7.54 (d, J=8.3 Hz, 1H), 5.80 (br. s., 1H), 3.59 (br. s., 2H), 3.17 (br. s., 2H), 2.81 (br. s., 4H), 2.17 (br. s., 6H), 1.90 (br. s., 2H), 1.78 (br. s., 2H), 1.59 (br. s., 2H), 1.23 (br. s., 2H), 0.8 (br. s., 2H).

### Example—S24: Synthesis of 2-((2-(2-aminoethyl)-1,2,3,4-tetrahydroisoquinolin-7-yl)amino)-8-cyclopentyl-7-oxo-7,8-dihydropyrido[2,3-d]pyrimidine-6-carbonitrile (Compound No. 65)

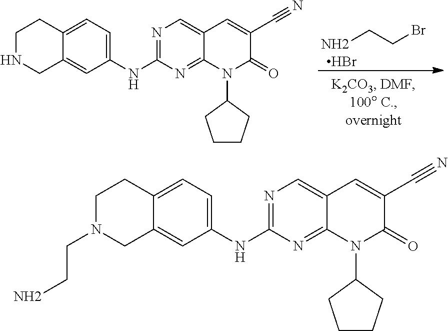

To a stirred solution of 8-cyclopentyl-7-oxo-2-((1,2,3,4-tetrahydroisoquinolin-7-yl)amino)-7,8-dihydropyrido[2,3-d]pyrimidine-6-carbonitrile (100 mg, 0.23 mmol, 1 equiv) in DMF (5 mL), was added K₂CO₃ (65 mg, 0.47 mmol, 2 equiv) and 2-bromoethan-1-amine.HBr (182 mg, 0.47 mmol, 2 equiv). The resultant reaction mixture was allowed to stir at 100° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 430 [M+H]⁺; ¹H NMR: (DMSO-d₆, 400 MHz): δ 8.83 (s, 1H), 8.56 (s, 1H), 7.55 (s, 1H), 7.37 (br. s., 1H), 7.11 (d, J=7.3 Hz, 1H), 5.81 (s, 1H), 3.59 (br. s., 2H), 2.92 (br. s., 2H), 2.81 (br. s., 2H), 2.69 (d, J=13.7 Hz, 2H), 2.19 (br. s., 2H), 2.03-2.14 (m, 2H), 1.92 (br. s., 2H), 1.81 (br. s., 2H), 1.59 (br. s., 2H).

### Example—S25: Synthesis of 2-((2-(2-aminoethyl)-1,2,3,4-tetrahydroisoquinolin-6-yl)amino)-8-cyclopentyl-7-oxo-7,8-dihydropyrido[2,3-d]pyrimidine-6-carbonitrile (Compound No. 66)

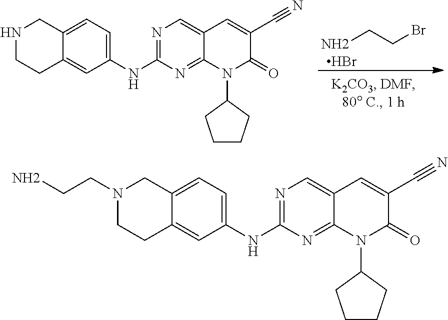

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.23 mmol, 1 equiv) in DMF (5 mL), was added K₂CO₃ (65 mg, 0.47 mmol, 2 equiv) and 2-bromoethan-1-amine.HBr (94 mg, 0.47 mmol, 2 equiv). The resultant reaction mixture was allowed to stir at 80° C. for 1 h. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to desired product. LCMS: 430 [M+H]⁺; ¹H NMR: (400 MHz, DMSO-d₆): δ 8.83 (s, 1H), 8.56 (s, 1H), 7.55 (s, 1H), 7.37 (br. s., 1H), 7.11 (d, J=7.3 Hz, 1H), 5.81 (s, 1H), 3.59 (br. s., 2H), 2.92 (br. s., 2H), 2.81 (br. s., 2H), 2.69 (d, J=13.7 Hz, 2H), 2.19 (br. s., 2H), 2.03-2.14 (m, 2H), 1.92 (br. s., 2H), 1.81 (br. s., 2H), 1.59 (br. s., 2H).

### Example—S26: Synthesis of 8-cyclopentyl-2-((2-(2-(dimethylamino)ethyl)-1-oxo-1,2,3,4-tetrahydroisoquinolin-7-yl)amino)-7-oxo-7,8-dihydropyrido[2,3-d]pyrimidine-6-carbonitrile (Compound No. 68)

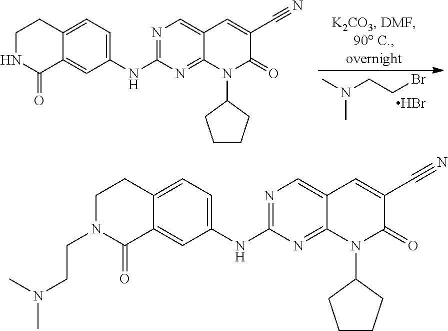

To a stirred solution of 8-cyclopentyl-7-oxo-2-((1-oxo-1,2,3,4-tetrahydroisoquinolin-7-yl)amino)-7,8-dihydropyrido[2,3-d]pyrimidine-6-carbonitrile (80 mg, 0.2 mmol, 1 equiv) in DMF (5 mL), was added K₂CO₃ (55 mg, 0.4 mmol, 2 equiv) and 2-bromo-N,N-dimethylethan-1-amine.HBr (93 mg, 0.4 mmol, 2 equiv). The resultant reaction mixture was allowed to stir at 90° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 472 [M+H]⁺; ¹HNMR: (400 MHz, DMSO-d₆) δ 8.52 (s, 1H), 8.21 (s, 1H), 8.02 (s, 1H), 7.79-7.74 (m, 1H), 7.44 (q, J=8.2 Hz, 1H), 5.21 (s, 1H), 4.13 (d, J=8.3 Hz, 2H), 3.49-3.36 (m, 2H), 2.95 (t, J=6.5 Hz, 2H), 2.16 (s, 6H), 1.89 (s, 2H), 1.61 (s, 2H), 1.42 (s, 2H), 1.29-1.21 (m, 2H), 1.16-1.03 (m, 2H).

### Example—S27: Synthesis of 2-(6-((6-cyano-8-cyclopentyl-7-oxo-7,8-dihydropyrido[2,3-d]pyrimidin-2-yl)amino)-3,4-dihydroisoquinolin-2(1H)-yl)acetamide (Compound No. 69)

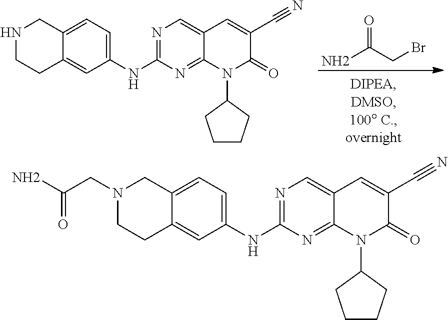

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.23 mmol, 1 equiv) in DMSO (3 mL), was added DIPEA (0.2 mL, 0.92 mmol, 4 equiv) and 2-bromoacetamide (64 mg, 0.47 mmol, 2 equiv). The resultant reaction mixture was allowed to stir at 100° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by recrystallization with methanol to afford desired product. LCMS: 444 [M+H]⁺; ¹HNMR: (400 MHz, DMSO-d₆) δ 10.49 (s, 1H), 8.83 (s, 1H), 8.56 (s, 1H), 7.64-7.58 (m, 1H), 7.34 (s, 1H), 7.27-7.22 (m, 1H), 7.14 (s, 1H), 7.05 (d, J=8.3 Hz, 1H), 5.83 (d, J=16.1 Hz, 1H), 3.61 (s, 2H), 3.04 (s, 2H), 2.85 (t, J=5.9 Hz, 2H), 2.72 (t, J=5.7 Hz, 2H), 2.54 (s, 2H), 2.20 (dq, J=14.7, 7.8 Hz, 2H), 2.03-1.87 (m, 1H), 1.80 (dq, J=11.0, 7.1, 4.7 Hz, 1H), 1.59 (h, J=7.9 Hz, 2H).

### Example—S28: Synthesis of 8-cyclopentyl-2-((2-(2-hydroxyacetyl)-1,2,3,4-tetrahydroisoquinolin-6-yl)amino)-7-oxo-7,8-dihydropyrido[2,3-d]pyrimidine-6-carbonitrile (Compound No. 70)

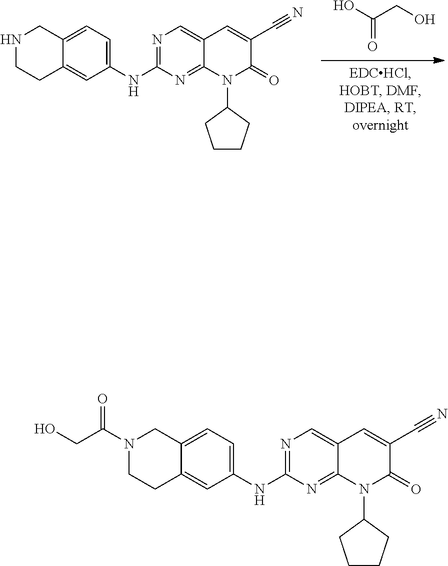 [SMILES: N#Cc1cc2cnc(Nc3ccc4c(c3)CCNC4)nc2n(C2CCCC2)c1=O ; O=C(O)CO ; N#Cc1cc2cnc(Nc3ccc4c(c3)CCN(C(=O)CO)C4)nc2n(C2CCCC2)c1=O]

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.26 mmol, 1 equiv) in DMF (5 mL), was added EDC.HCl (72 mg, 0.38 mmol, 1.5 equiv), HOBt (51 mg, 0.38 mmol, 1.5 equiv), DIPEA (0.2 mL, 0.78 mmol, 3 equiv) and 2-hydroxyacetic acid (30 mg, 0.38 mmol, 1.5 equiv). The reaction mixture was allowed to stir at RT for overnight. Progress of the reaction was monitored by LCMS. After completion of the reaction, the reaction mixture was filtered and washed with water and purified by Reverse phase chromatography to get desired product. LCMS: 445 [M+H]⁺; ¹H NMR: (400 MHz, DMSO-d₆) δ 10.53 (s, 1H), 8.83 (s, 1H), 8.55 (s, 1H), 7.63 (s, 1H), 7.48-7.38 (m, 1H), 7.18 (dd, J=17.0, 8.3 Hz, 1H), 5.82 (s, 1H), 4.61 (d, J=9.9 Hz, 2H), 4.53 (s, 1H), 4.17 (t, J=4.3 Hz, 2H), 3.69 (d, J=6.1 Hz, 1H), 3.58 (d, J=6.1 Hz, 1H), 2.80 (dt, J=28.5, 5.9 Hz, 2H), 2.19 (dq, J=14.9, 7.5 Hz, 2H), 1.91 (s, 2H), 1.80 (qd, J=10.8, 6.9, 4.8 Hz, 2H), 1.60 (dq, J=10.9, 5.8, 4.7 Hz, 2H).

### Example—S29: Synthesis of 2-((2-acetyl-1,2,3,4-tetrahydroisoquinolin-6-yl)amino)-8-cyclopentyl-7-oxo-7,8-dihydropyrido[2,3-d]pyrimidine-6-carbonitrile (Compound No. 71)

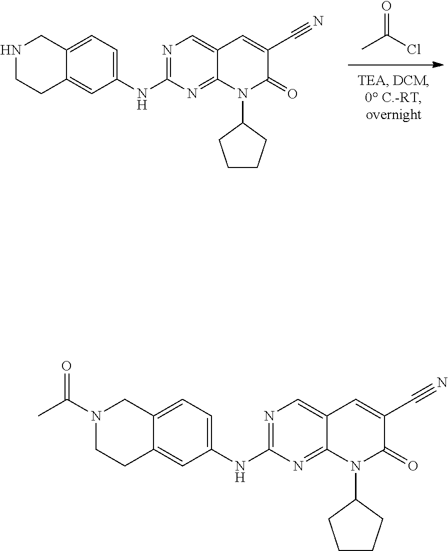

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.23 mmol, 1 equiv) in DCM (5 mL), was added TEA (0.1 mL, 0.66 mmol, 3 equiv). Reaction mixture was cooled to 0° C., followed by the addition of Acetyl chloride (0.2 mL, 0.34 mmol, 1.5 equiv) and the resultant reaction mixture was allowed to stir for overnight at RT. Progress of the reaction was monitored by LCMS. After completion of the reaction, the reaction mixture was diluted with water (15 mL) and extracted with ethyl acetate (20 mL×2). Organic layer was washed with water (10 mL) and brine solution (10 mL). Organic layer was dried over anhydrous sodium sulphate and concentrated under reduced pressure to obtain crude, was triturated by methanol to afford desired product. LCMS: 429 [M+H]⁺; ¹HNMR: (DMSO-d₆, 400 MHz): d=10.53 (br. s., 1H), 8.84 (s, 1H), 8.57 (s, 1H), 7.63 (br. s., 1H), 7.44 (br. s., 1H), 7.18 (d, J=8.3 Hz, 1H), 5.82 (br. s., 1H), 4.62 (s, 1H), 4.56 (s, 1H), 3.55-3.78 (m, 2H), 2.85 (t, J=5.5 Hz, 1H), 2.74 (t, J=5.7 Hz, 1H), 2.11-2.28 (m, 2H), 2.08 (s, 3H), 1.91 (br. s., 2H), 1.81 (br. s., 2H).

### Example—S30: Synthesis of 8-cyclopentyl-2-((2-(3-(dimethylamino)propyl)-1,2,3,4-tetrahydroisoquinolin-6-yl)amino)-7-oxo-7,8-dihydropyrido[2,3-d]pyrimidine-6-carbonitrile (Compound No. 72)

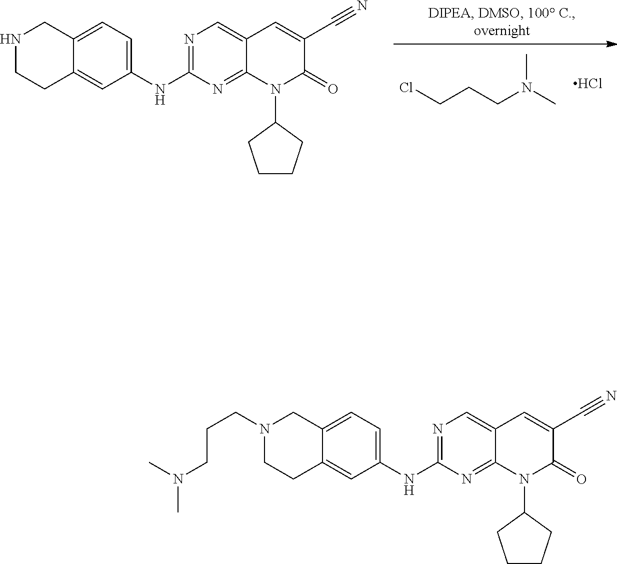

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.23 mmol, 1 equiv) in DMF (5 mL), was added DIPEA (0.8 mL, 0.47 mmol, 2 equiv) and 3-chloro-N,N-dimethylpropan-1-amine hydrochloride (75 mg, 0.47 mmol, 2 equiv). The resultant reaction mixture was allowed to stir at 100° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 472 [M+H]⁺; ¹H NMR: (400 MHz, DMSO-d₆): d=8.83 (s, 1H), 8.57 (s, 1H), 8.16 (br. s., 1H), 7.60 (br. s., 1H), 7.35 (br. s., 1H), 7.06 (d, J=7.9 Hz, 1H), 5.82 (br. s., 1H), 3.55 (br. s., 4H), 2.81 (br. s., 2H), 2.68 (br. s., 4H), 2.44 (br. s., 6H), 2.19 (br. s., 2H), 1.91 (br. s., 2H), 1.79 (br. s., 4H), 1.59 (br. s., 2H).

### Example—S31: Synthesis of 8-cyclopentyl-2-((1-(1-(2-(dimethylamino)ethyl)piperidin-4-yl)-2-oxo-1,2-dihydropyridin-4-yl)amino)-7-oxo-7,8-dihydropyrido[2,3-d]pyrimidine-6-carbonitrile (Compound No. 73)

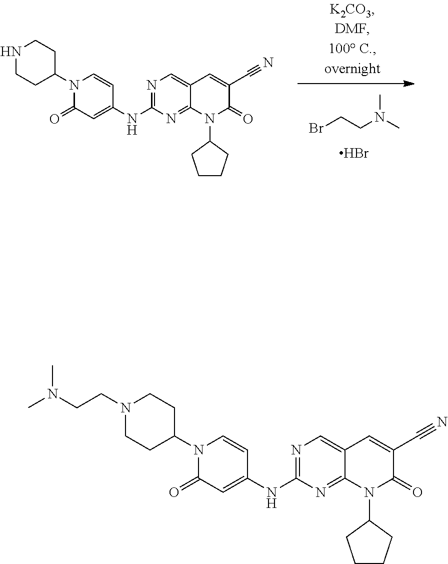

To a stirred solution of 8-cyclopentyl-7-oxo-2-((2-oxo-1-(piperidin-4-yl)-1,2-dihydropyridin-4-yl)amino)-7,8-dihydropyrido[2,3-d]pyrimidine-6-carbonitrile (100 mg, 0.23 mmol, 1 equiv) in DMF (5 mL), was added K₂CO₃ (65 mg, 0.47 mmol, 2 equiv) and 2-bromo-N,N-dimethylethan-1-amine hydrobromide (182 mg, 0.47 mmol, 2 equiv). The resultant reaction mixture was allowed to stir at 100° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 503 [M+H]⁺; ¹H NMR (DMSO-d₆, 400 MHz): δ 10.58 (br. s., 1H), 8.95 (s, 1H), 8.66 (s, 1H), 7.68 (d, J=7.9 Hz, 2H), 7.03 (d, J=2.2 Hz, 2H), 6.57 (d, J=5.7 Hz, 1H), 5.69-5.96 (m, 1H), 4.41-4.67 (m, 1H), 3.02 (d, J=11.0 Hz, 2H), 2.47 (br. s., 6H), 2.33 (br. s., 1H), 2.24 (s, 4H), 2.18 (br. s., 1H), 2.08 (t, J=11.2 Hz, 2H), 1.99 (br. s., 2H), 1.72-1.90 (m, 4H), 1.66 (d, J=11.8 Hz, 2H).

### Example—S32: Synthesis of 8-cyclopentyl-2-((2-(methylsulfonyl)-1,2,3,4-tetrahydroisoquinolin-6-yl)amino)-7-oxo-7,8-dihydropyrido[2,3-d]pyrimidine-6-carbonitrile (Compound No. 74)

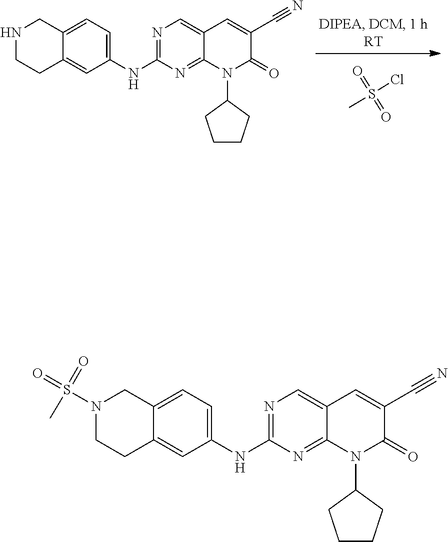

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.23 mmol, 1 equiv) in DCM (4 mL), was added TEA (0.1 mL, 0.721 mmol, 3 equiv) followed by addition of mesyl chloride (65 mg, 0.47 mmol, 2 equiv) at 0° C. The resultant reaction mixture was allowed to stir at RT for 1 h. Progress of the reaction was monitored by LCMS. After completion of the reaction, the reaction mixture was diluted with water (15 mL) and extracted with ethyl acetate (20 mL×2). Organic layer was washed with water (10 mL) and brine solution (10 mL). Organic layer was dried over anhydrous sodium sulphate and concentrated under reduced pressure to obtain crude, which was purified by reverse phase HPLC to afford desired product. LCMS: 465 [M+H]⁺; ¹H NMR: (400 MHz, DMSO-d₆) δ 10.54 (s, 1H), 8.85 (s, 1H), 8.57 (s, 1H), 7.65 (s, 1H), 7.45 (s, 1H), 7.19 (d, J=8.4 Hz, 1H), 5.83 (s, 1H), 4.34 (s, 2H), 3.44 (t, J=5.9 Hz, 2H), 2.95 (s, 3H), 2.90 (t, J=6.0 Hz, 2H), 2.19 (dq, J=14.6, 7.7 Hz, 2H), 1.92 (s, 2H), 1.81 (dq, J=4.1 Hz, 2H), 1.60 (dt, J=10.2, 6.8 Hz, 2H).

### Example—S33: Synthesis of 8-cyclopentyl-2-((2-(2-methoxyethyl)-1,2,3,4-tetrahydroisoquinolin-6-yl)amino)-7-oxo-7,8-dihydropyrido[2,3-d]pyrimidine-6-carbonitrile (Compound No. 75)

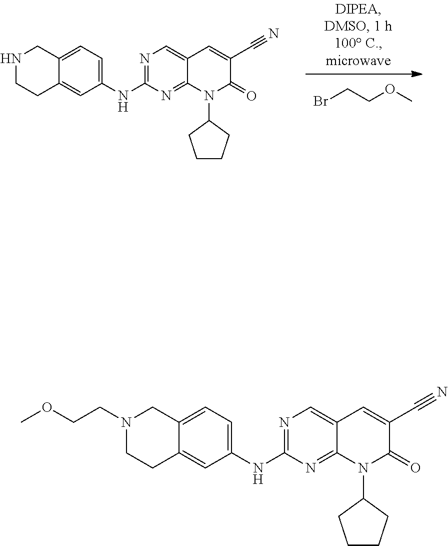

To a stirred solution of 8-cyclopentyl-7-oxo-2-((1,2,3,4-tetrahydroisoquinolin-6-yl)amino)-7,8-dihydropyrido[2,3-d]pyrimidine-6-carbonitrile (100 mg, 0.23 mmol, 1 equiv) in DMSO (5 mL), was added DIPEA (0.2 mL, 0.92 mmol, 4 equiv) and 1-bromo-2-methoxyethane (65 mg, 0.47 mmol, 2 equiv). The resultant reaction mixture was allowed to stir at 100° C. for 1 h in microwave. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 445 [M+H]⁺; ¹H NMR (DMSO-d₆, 400 MHz): δ 10.49 (br. s., 1H), 8.83 (s, 1H), 8.56 (s, 1H), 7.58 (br. s., 1H), 7.33 (br. s., 1H), 7.03 (d, J=7.9 Hz, 1H), 5.82 (br. s., 1H), 3.44-3.63 (m, 2H), 3.26 (s, 3H), 3.16 (d, J=5.3 Hz, 2H), 2.79 (br. s., 2H), 2.70 (br. s., 2H), 2.64 (t, J=5.5 Hz, 2H), 2.19 (br. s., 2H), 1.89 (br. s., 2H), 1.81 (br. s., 2H), 1.58 (br. s., 2H).

### Example—S34: Synthesis of 8-cyclopentyl-2-((2-(2-(dimethylamino)ethyl)-4,4-dimethyl-1,2,3,4-tetrahydroisoquinolin-7-yl)amino)-7-oxo-7,8-dihydropyrido[2,3-d]pyrimidine-6-carbonitrile (Compound No. 76)

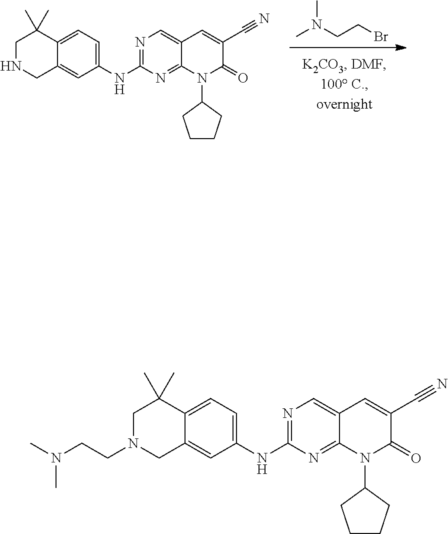

To a stirred solution of 8-cyclopentyl-2-((4,4-dimethyl-1,2,3,4-tetrahydroisoquinolin-7-yl)amino)-7-oxo-7,8-dihydropyrido[2,3-d]pyrimidine-6-carbonitrile (100 mg, 0.24 mom, 1 equiv) in DMF (5 mL), was added Potassium carbonate (50 mg, 0.36 mmol, 1.5 equiv), and 2-bromo-N,N-dimethylethan-1-amine (92 mg, 0.72 mmol, 3 equiv). The reaction mixture was allowed to stir at 100° C. for overnight. Progress of the reaction was monitored by LCMS. After completion of the reaction, the reaction mixture was diluted with water (10 mL) and was extracted with ethyl acetate (10 mL). Organic layer was dried over anhydrous Na2SO4, evaporated in vacuo. Product was purified by reverse phase HPLC. LCMS: 486 [M+H]⁺; ¹HNMR: (400 MHz, DMSO-d₆) δ 8.81 (s, 1H), 8.53 (s, 1H), 7.43 (s, 1H), 7.36 (q, J=8.4 Hz, 2H), 5.83-5.73 (m, 1H), 3.20 (s, 2H), 2.74 (s, 8H), 2.17 (s, 2H), 1.82 (t, J=17.6 Hz, 3H), 1.57 (d, J=9.0 Hz, 2H), 1.25 (s, 6H).

### Example—S35: Synthesis of 8-cyclopentyl-7-oxo-2-((5,6,7,8-tetrahydro-1,6-naphthyridin-3-yl)amino)-7,8-dihydropyrido[2,3-d]pyrimidine-6-carbonitrile (Compound No. 77)

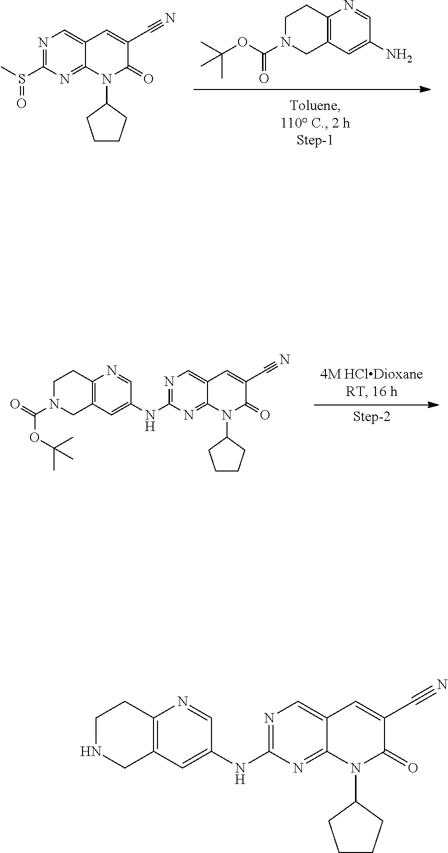 [SMILES: CS(=O)c1ncc2cc(C#N)c(=O)n(C3CCCC3)c2n1 ; CC(C)(C)OC(=O)N1CCc2ncc(N)cc2C1 ; CC(C)(C)OC(=O)N1CCc2ncc(Nc3ncc4cc(C#N)c(=O)n(C5CCCC5)c4n3)cc2C1 ; N#Cc1cc2cnc(Nc3cnc4c(c3)CNCC4)nc2n(C2CCCC2)c1=O]

Step—1: Synthesis of tert-butyl 3-((6-cyano-8-cyclopentyl-7-oxo-7,8-dihydropyrido[2,3-d]pyrimidin-2-yl)amino)-7,8-dihydro-1,6-naphthyridine-6(5H)-carboxylate: To a suspension of 8-cyclopentyl-2-(methylsulfinyl)-7-oxo-7,8-dihydropyrido[2,3-d]pyrimidine-6-carbonitrile (75 mg, 0.248 mmol, 1 equiv) in toluene (3 mL), was added tert-butyl 3-amino-7,8-dihydro-1,6-naphthyridine-6(5H)-carboxylate (67.7 mg, 0.273 mmol, 1.1 eqiv). Resultant reaction mixture was stirred at 110° C. for 2 h. Reaction was monitored by LCMS. After completion of reaction, reaction mass concentrated under reduced pressure. Product was triturated from methanol to afford desired product. LCMS: 488 [M+H]⁺

Step—2: Synthesis of 8-cyclopentyl-7-oxo-2-((5,6,7,8-tetrahydro-1,6-naphthyridin-3-yl)amino)-7,8-dihydropyrido[2,3-d]pyrimidine-6-carbonitrile: To a solution of tert-butyl 7-[(6-cyano-8-cyclopentyl-7-oxo-pyrido[2,3-d]pyrimidin-2-yl)amino]-3,4-dihydro-1H-isoquinoline-2-carboxylate (40 mg, 0.1 mmol, 1 eqiv) in dioxane (1 mL) was added to the 4M HCl in dioxane (2 mL). The resultant reaction mixture was stirred at RT for 16 h. Reaction was monitored by LCMS. After completion of reaction, the reaction mass was concentrated under reduced pressure to afford desired product. LCMS: 388 [M+H]⁺; ¹H NMR: (400 MHz, DMSO-d6) δ 10.86 (s, 1H), 9.79 (s, 2H), 8.92 (s, 1H), 8.83 (d, J=2.4 Hz, 1H), 8.66 (s, 1H), 8.16 (s, 1H), 5.81 (d, J=12.9 Hz, 1H), 4.36 (t, J=4.2 Hz, 2H), 3.54-3.44 (m, 2H), 3.16 (t, J=6.3 Hz, 2H), 2.18 (dq, J=14.6, 7.8 Hz, 2H), 1.95 (s, 2H), 1.83 (dt, J=11.3, 7.6 Hz, 2H), 1.63 (p, J=5.9, 4.6 Hz, 2H).

### Example—S36: Synthesis of 8-cyclopentyl-7-oxo-2-((2,3,4,5-tetrahydro-1H-benzo[c]azepin-8-yl)amino)-7,8-dihydropyrido[2,3-d]pyrimidine-6-carbonitrile (Compound No. 78)

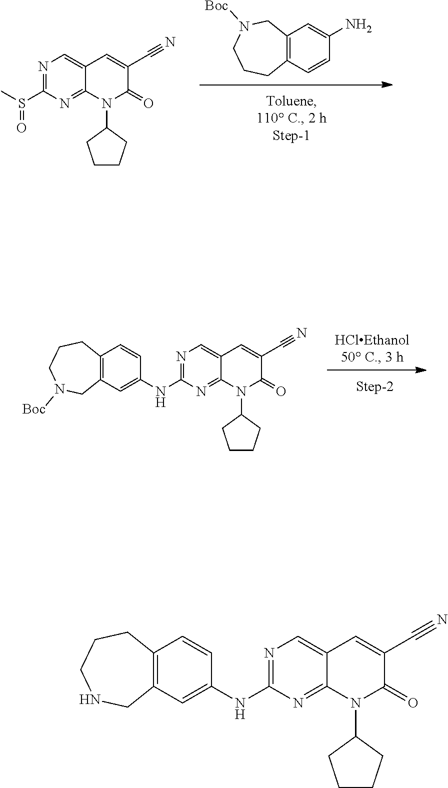 [SMILES: CS(=O)c1ncc2cc(C#N)c(=O)n(C3CCCC3)c2n1 ; CC(C)(C)OC(=O)N1CCCc2ccc(N)cc2C1 ; CC(C)(C)OC(=O)N1CCCc2ccc(Nc3ncc4cc(C#N)c(=O)n(C5CCCC5)c4n3)cc2C1 ; N#Cc1cc2cnc(Nc3ccc4c(c3)CNCCC4)nc2n(C2CCCC2)c1=O]

Step—1: Synthesis of tert-butyl 8-((6-cyano-8-cyclopentyl-7-oxo-7,8-dihydropyrido[2,3-d]pyrimidin-2-yl)amino)-1,3,4,5-tetrahydro-2H-benzo[c]azepine-2-carboxylate: To a suspension of 8-cyclopentyl-2-(methylsulfinyl)-7-oxo-7,8-dihydropyrido[2,3-d]pyrimidine-6-carbonitrile (100 mg, 0.33 mmol, 1 eqiv) in toluene (4 mL), was added tert-butyl 8-amino-1,3,4,5-tetrahydro-2H-benzo[c]azepine-2-carboxylate (95 mg, 0.36 mmol, 1.1 equiv). Resultant reaction mixture was stirred at 110° C. for 2 h. Reaction was monitored by LCMS. After completion of reaction, reaction mass was concentrated under reduced pressure. Product was triturated from methanol to afford desired product. LCMS: 501 [M+H]⁺

Step—2: Synthesis of 8-cyclopentyl-7-oxo-2-((2,3,4,5-tetrahydro-1H-benzo[c]azepin-8-yl)amino)-7,8-dihydropyrido[2,3-d]pyrimidine-6-carbonitrile: To a solution of tert-butyl 8-((6-cyano-8-cyclopentyl-7-oxo-7,8-dihydropyrido[2,3-d]pyrimidin-2-yl)amino)-1,3,4,5-tetrahydro-2H-benzo[c]azepine-2-carboxylate (80 mg, 0.1 mmol, 1 equiv) in HCl in ethanol (1.25 M, 4 mL) was stirred at 50° C. for 3 h. Reaction was monitored by LCMS. After completion of reaction, product precipitates out was filtered and washed with ethanol (5 mL) to afford desired product. LCMS: 401 [M+H]⁺; ¹H NMR: (400 MHz, DMSO-d₆): δ 10.62 (br. s., 1H), 9.22 (br. s, 1H), 8.6 (s, 1H), 8.86 (s, 1H), 7.8 (s., 1H), 7.59 (d, 1H), 7.25 (d, 1H), 5.81 (br. s, 1H), 4.23 (br. s., 2H), 2.97 (br. s., 2H), 2.1-2.22 (m, 2H), 1.8-2 (m, 8H), 1.6 (br. s., 2H).

### Example—S37: Synthesis of 8-cyclopentyl-7-oxo-2-((1-oxo-2, 3, 4, 5-tetrahydro-1H-benzo[c]azepin-8-yl) amino)-7, 8-dihydropyrido [2, 3-d]pyrimidine-6-carbonitrile (Compound No. 79)

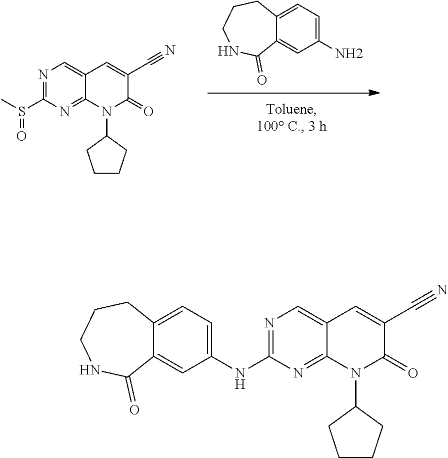 [SMILES: CS(=O)c1ncc2cc(C#N)c(=O)n(C3CCCC3)c2n1 ; Nc1ccc2c(c1)C(=O)NCCC2 ; N#Cc1cc2cnc(Nc3ccc4c(c3)C(=O)NCCC4)nc2n(C2CCCC2)c1=O]

To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 8-amino-2,3,4,5-tetrahydro-1H-benzo[c]azepin-1-one (64 mg, 0.36 mmol, 1.1 equiv). The resultant reaction mixture was stirred at 100° C. for 3 h. Progress of the reaction was monitored by LCMS. After completion of the reaction, solvent was removed completely under reduced pressure to obtain crude compound, which was purified by recrystallization with methanol to obtain desired product. LCMS: 415 [M+H]⁺; ¹H NMR: (DMSO-d₆, 400 MHz): δ 10.64 (br. s., 1H), 8.85 (s, 1H), 8.58 (s, 1H), 8.14 (br. s., 1H), 8.14-8.08 (br. s., 2H), 7.58 (br. s., 1H), 7.25 (d, J=8.3 Hz, 1H), 5.89 (br. s., 1H), 2.93 (d, J=6.1 Hz, 2H), 2.59-2.77 (m, 2H), 2.15 (br. s., 2H), 1.88-1.84 (d, J=6.6 Hz, 6H), 1.64 (br. s., 2H).

### Example—S38: Synthesis of 8-(sec-butyl)-7-oxo-2-((1,2,3,4-tetrahydroisoquinolin-6-yl)amino)-7,8-dihydropyrido[2,3-d]pyrimidine-6-carbonitrile (Compound No. 80)

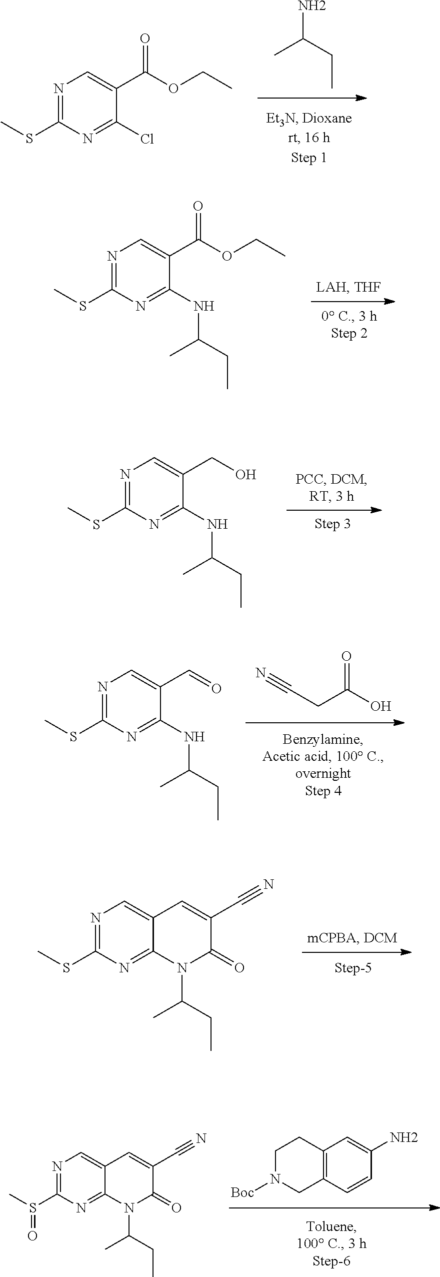

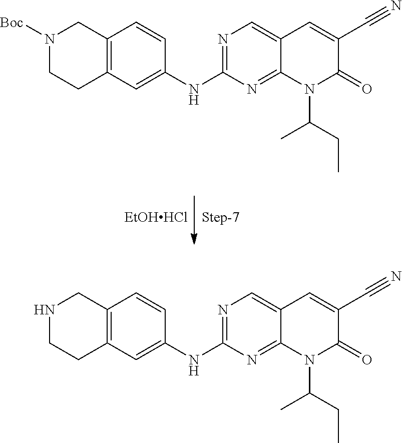 [SMILES: "EtOH·HCl" ; CCC(C)n1c(=O)c(C#N)cc2cnc(Nc3ccc4c(c3)CCN(C(=O)OC(C)(C)C)C4)nc21 ; "Step-7" ; CCC(C)n1c(=O)c(C#N)cc2cnc(Nc3ccc4c(c3)CCNC4)nc21]

Step—1: Synthesis of ethyl 4-(sec-butylamino)-2-(methylthio)pyrimidine-5-carboxylate: To a stirred solution of ethyl 4-chloro-2-(methylthio)pyrimidine-5-carboxylate (2000 mg, 8.58 mmol, 1 equiv) in Dioxane (20 mL), was added Et₃N (3.6 mL, 10.3 mmol, 1.2 equiv) and butan-2-amine (752 mg, 10.3 mmol, 1.2 equiv) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to desired product. LCMS: 270 [M+H]⁺

Step—2: Synthesis of (4-(sec-butylamino)-2-(methylthio)pyrimidin-5-yl)methanol: To a stirred solution of ethyl 4-(sec-butylamino)-2-(methylthio)pyrimidine-5-carboxylate (2000 mg, 7.4 mmol, 1 equiv) in THF (50 mL), was added LAH (565 mg, 14.8 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium hydroxide (100 mL) at 0° C. and extracted with ethyl acetate (150 mL×2). Organic layer was washed with water (100 mL) and brine solution (100 mL). Organic layer was dried over anhydrous sodium sulphate and concentrated under reduced pressure to obtain desired product. LCMS: 228 [M+H]⁺

Step—3: Synthesis of 4-(sec-butylamino)-2-(methylthio)pyrimidine-5-carbaldehyde: To a stirred solution of (4-(sec-butylamino)-2-(methylthio) pyrimidin-5-yl) methanol (1400 mg, 6.16 mmol, 1 equiv) in DCM (30 mL), was added PCC (1332 mg, 6.16 mmol, 1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain desired product. LCMS: 226 [M+H]⁺

Step—4: Synthesis of 8-(sec-butyl)-2-(methylthio)-7-oxo-7,8-dihydropyrido[2,3-d]pyrimidine-6-carbonitrile: To a stirred solution of 4-(sec-butylamino)-2-(methylthio)pyrimidine-5-carbaldehyde (1000 mg, 5.33 mmol, 1 equiv) in Acetic acid (15 mL), was added Cyanoacetic acid (453 mg, 5.33 mmol, 1.2 equiv) and Benzyl amine (0.1 mL, 0.44, 0.1 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain desired product. LCMS: 275 [M+H]⁺

Step—5: Synthesis of 8-(sec-butyl)-2-(methylsulfinyl)-7-oxo-7,8-dihydropyrido[2,3-d]pyrimidine-6-carbonitrile: To a stirred solution of 8-(sec-butyl)-2-(methylthio)-7-oxo-7,8-dihydropyrido[2,3-d]pyrimidine-6-carbonitrile (150 mg, 0.54 mmol, 1 equiv) in DCM (5 mL), was added m-CPBA (133 mg, 0.76 mmol, 1.4 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain desired product. LCMS: 291 [M+H]⁺

Step—6: Synthesis of tert-butyl 6-((8-(sec-butyl)-6-cyano-7-oxo-7,8-dihydropyrido[2,3-d]pyrimidin-2-yl)amino)-3,4-dihydroisoquinoline-2(1H)-carboxylate: To a stirred solution of 8-(sec-butyl)-2-(methylsulfinyl)-7-oxo-7,8-dihydropyrido[2,3-d]pyrimidine-6-carbonitrile (100 mg, 0.36 mmol, 1 equiv) in toluene (5 mL), was added tert-butyl 6-amino-3,4-dihydroisoquinoline-2(1H)-carboxylate (100 mg, 0.4 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain crude compound, which was purified by recrystallization with methanol to obtain desired product. LCMS: 475 [M+H]⁺

Step—7: Synthesis of 8-(sec-butyl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((8-(sec-butyl)-6-cyano-7-oxo-7,8-dihydropyrido[2,3-d]pyrimidin-2-yl)amino)-3,4-dihydroisoquinoline-2(1H)-carboxylate (80 mg, 0.16 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain residue, which was dried under lyophilizer to obtain desired product. LCMS: 375 [M+H]⁺; ¹H NMR: (400 MHz, DMSO-d₆): 10.64 (br. s., 1H), 9.35 (br. s., 1H), 8.87 (s, 1H), 8.61 (s, 1H), 7.68 (br. s., 1H), 7.52 (br. s., 1H), 7.23 (d, J=8.8 Hz, 1H), 5.50 (br. s., 1H), 4.24 (br. s., 2H), 3.27-3.41 (m, 2H), 3.01 (t, J=6.1 Hz, 2H), 2.15 (br. s., 1H), 1.93 (dt, J=13.9, 6.9 Hz, 1H), 1.51 (d, J=5.3 Hz, 3H), 0.80 (br. s., 3H).

### Example—S39: Synthesis of 8-isobutyl-7-oxo-2-((1,2,3,4-tetrahydroisoquinolin-6-yl)amino)-7,8-dihydropyrido[2,3-d]pyrimidine-6-carbonitrile (Compound No. 81)

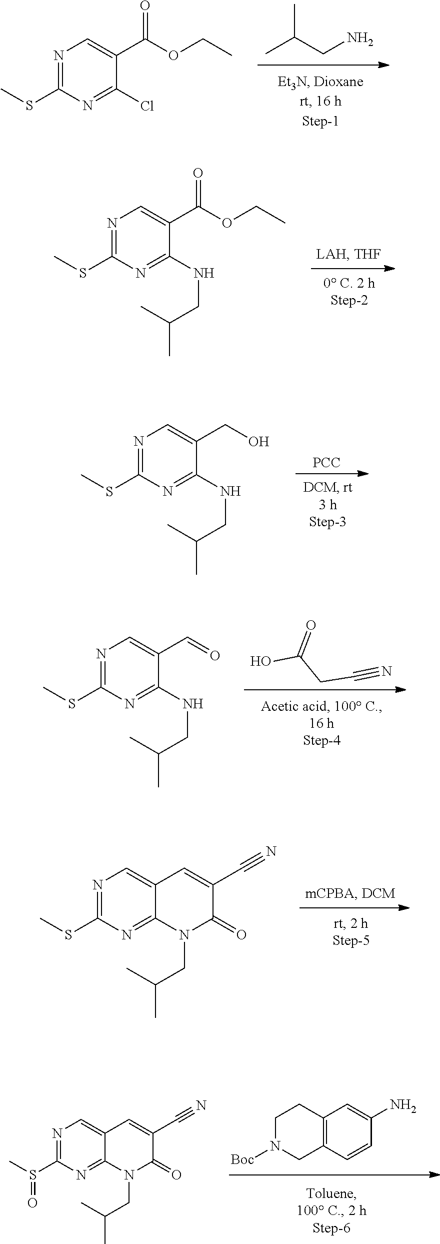

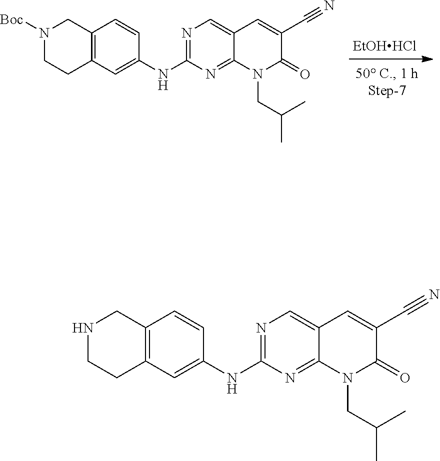 [SMILES: "EtOH·HCl" ; CC(C)Cn1c(=O)c(C#N)cc2cnc(Nc3ccc4c(c3)CCN(C(=O)OC(C)(C)C)C4)nc21 ; "50° C., 1 hStep-7" ; CC(C)Cn1c(=O)c(C#N)cc2cnc(Nc3ccc4c(c3)CCNC4)nc21]

Step—1: Synthesis of ethyl 4-(isobutylamino)-2-(methylthio)pyrimidine-5-carboxylate: To a stirred solution of ethyl 4-chloro-2-(methylthio)pyrimidine-5-carboxylate (5000 mg, 21.55 mmol, 1 equiv) in Dioxane (60 mL), was added Et₃N (5.99 mL, 43.10 mmol, 2.0 equiv) and 2-methylpropan-1-amine (1890 mg, 25.86 mmol, 1.2 equiv) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain desired product. LCMS: 270 [M+H]⁺

Step—2: Synthesis of (4-(isobutylamino)-2-(methylthio)pyrimidin-5-yl)methanol: To a stirred solution of ethyl 4-(isobutylamino)-2-(methylthio)pyrimidine-5-carboxylate (5000 mg, 18.58 mmol, 1.0 equiv) in THF (50 mL), was added LAH (1410 mg, 37.17 mmol, 2 equiv) at 0° C. The reaction mixture was allowed to stir at RT for 3 h. Progress of the reaction was monitored by TLC and LCMS. After completion of the reaction, the reaction mixture was quenched with saturated solution of sodium hydroxide (20 mL) at 0° C. and extracted with ethyl acetate (150 mL×2). Organic layer was washed with water (100 mL) and brine solution (100 mL). Organic layer was dried over anhydrous sodium sulphate and concentrated under reduced pressure to obtain desired product. LCMS: 228 [M+H]⁺

Step—3: Synthesis of 4-(isobutylamino)-2-(methylthio)pyrimidine-5-carbaldehyde: To a stirred solution of (4-(isobutylamino)-2-(methylthio)pyrimidin-5-yl)methanol (3400 mg, 14.98 mmol, 1 equiv) in DCM (30 mL), was added PCC (3220 mg, 14.98 mmol, 1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain desired product. LCMS: 226 [M+H]⁺

Step—4: Synthesis of 8-isobutyl-2-(methylthio)-7-oxo-7,8-dihydropyrido[2,3-d]pyrimidine-6-carbonitrile: To a stirred solution of 4-(isobutylamino)-2-(methylthio)pyrimidine-5-carbaldehyde (2200 mg, 9.78 mmol, 1 equiv) in Acetic acid (25 mL), was added cyanoacetic acid (1080 mg, 12.71 mmol, 1.3 equiv) and Benzyl amine (0.12 mL, 1.17 mmol, 0.12 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain desired product. LCMS: 275 [M+H]⁺

Step—5: Synthesis of 8-isobutyl-2-(methylsulfinyl)-7-oxo-7,8-dihydropyrido[2,3-d]pyrimidine-6-carbonitrile: To a stirred solution of 8-isobutyl-2-(methylthio)-7-oxo-7,8-dihydropyrido[2,3-d]pyrimidine-6-carbonitrile (200 mg, 0.73 mmol, 1 equiv) in DCM (5 mL), was added m-CPBA (164 mg, 0.95 mmol, 1.3 equiv) at RT. Then the reaction mixture was allowed to stir for 2 h. Progress of the reaction was monitored by LCMS. After completion of the reaction, the reaction mixture was diluted with DCM (50 mL) and washed with saturated solution of NaHCO₃, water (50 mL), brine solution (50 mL). Organic layer was dried over anhydrous sodium sulphate and concentrated under reduced pressure to obtain desired product. LCMS: 290 [M+H]⁺

Step—6: Synthesis of tert-butyl 6-((6-cyano-8-isobutyl-7-oxo-7,8-dihydropyrido[2,3-d]pyrimidin-2-yl)amino)-3,4-dihydroisoquinoline-2(1H)-carboxylate: To a stirred solution of 8-isobutyl-2-(methylsulfinyl)-7-oxo-7,8-dihydropyrido[2,3-d]pyrimidine-6-carbonitrile (60 mg, 0.207 mmol, 1 equiv) in toluene (5 mL), was added tert-butyl 6-amino-3,4-dihydroisoquinoline-2(1H)-carboxylate (56 mg, 0.23 mmol, 1.1 equiv). The resultant reaction mixture was allowed to stir at 100° C. for 2 h. Progress of the reaction was monitored by LCMS. After completion of the reaction, solid observed was filtered and dried under vacuum to obtain crude compound, which was purified by recrystallization with methanol to obtain desired product. LCMS: 475 [M+H]⁺

Step—7: Synthesis of 8-isobutyl-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-isobutyl-7-oxo-7,8-dihydropyrido[2,3-d]pyrimidin-2-yl)amino)-3,4-dihydroisoquinoline-2(1H)-carboxylate (50 mg, 0.1 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain residue, which was dried under lyophilizer to obtain desired product. LCMS: 375 [M+H]⁺; ¹H NMR: (DMSO-d₆, 400 MHz): ¹H NMR (400 MHz, DMSO-d₆): δ 10.66 (br. s., 1H), 9.25 (br. s., 2H), 8.88 (s, 1H), 8.65 (s, 1H), 7.69 (br. s., 1H), 7.64 (s, 1H), 7.24 (d, J=8.3 Hz, 1H), 4.24 (br. s., 2H), 4.14 (d, J=7.9 Hz, 2H), 3.38 (br. s., 2H), 2.98-3.07 (m, 2H), 2.21-2.29 (m, 1H), 0.83-0.95 (m, 6H).

### Example—S40: Synthesis of 8-cyclopentyl-7-oxo-2-((1-oxo-2,3,4,5-tetrahydro-1H-benzo[c]azepin-7-yl)amino)-7,8-dihydropyrido[2,3-d]pyrimidine-6-carbonitrile (Compound No. 82)

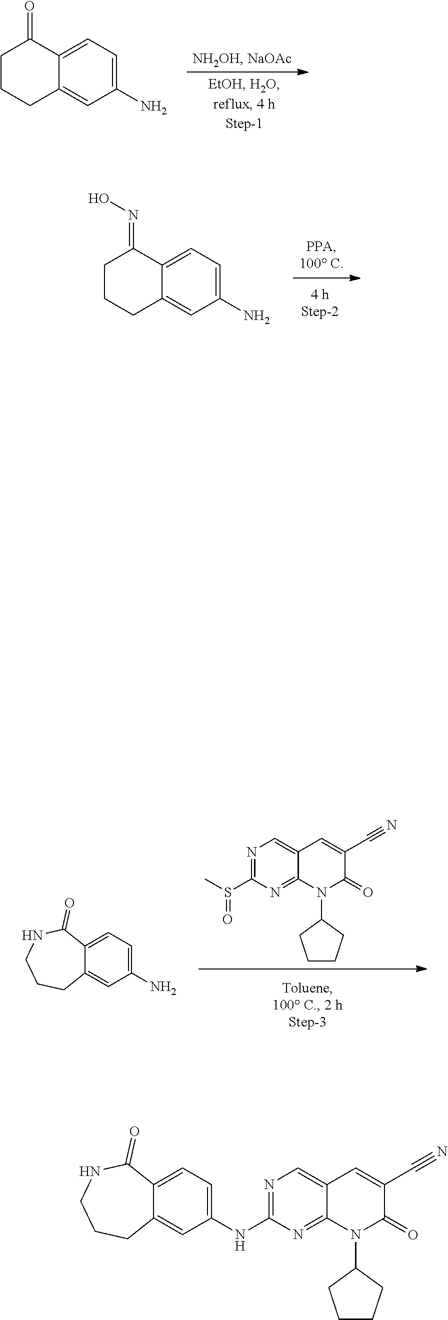 [SMILES: Nc1ccc2c(c1)CCCC2=O ; Nc1ccc2c(c1)CCC/C2=N\O ; Nc1ccc2c(c1)CCCNC2=O ; CS(=O)c1ncc2cc(C#N)c(=O)n(C3CCCC3)c2n1 ; N#Cc1cc2cnc(Nc3ccc4c(c3)CCCNC4=O)nc2n(C2CCCC2)c1=O]

Step—1: Synthesis of (E/Z)-6-amino-3,4-dihydronaphthalen-1(2H)-one oxime: To the solution of 6-amino-3,4-dihydronaphthalen-1(2H)-one (1000 mg, 0.062 mmol, 1 eq) in 7.5 mL Ethanol and 2.5 mL water was added hydroxylamine (430 mg, 0.062 mmol, 1 eq) and sodium acetate (1260 mg, 0.155 mmol, 2.5 eq) and the reaction mixture was heated at reflux for 4 h. After completion of the reaction (monitored by LCMS), reaction mixture was cooled to RT and diluted with 10 mL water and the precipitates formed were filtered to obtain desired product. LCMS: 177 [M+H]⁺

Step—2: Synthesis of 7-amino-2,3,4,5-tetrahydro-1H-benzo[c]azepin-1-one: (E/Z)-6-amino-3,4-dihydronaphthalen-1(2H)-one (600 mg, 0.034 mmoles, 1 eq) and PPA (10 mL) were mixed and heated to 100° C. for 4 h until the mixture becomes homogenous. After completion of the reaction (monitored by LCMS), reaction mixture was cooled to RT and diluted with 10 mL water and 15% aq. NaOH solution until the mixture becomes basic. Reaction mixture was then extracted with ethylacetate 20 mL, dried over sodium sulfate and concentrated under vacuum to obtain desired product. LCMS: 177 [M+H]⁺

Step—3: Synthesis of 8-cyclopentyl-7-oxo-2-((1-oxo-2,3,4,5-tetrahydro-1H-benzo[c]azepin-7-yl)amino)-7,8-dihydropyrido[2,3-d]pyrimidine-6-carbonitrile: To a stirred solution of 8-cyclobutyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 7-amino-2,3,4,5-tetrahydro-1H-benzo[c]azepin-1-one (64 mg, 0.69 mmol, 1.1 equiv). The resultant reaction mixture was allowed to stir at 100° C. for 2 h. Progress of the reaction was monitored by LCMS. After completion of the reaction, solid observed was filtered and dried under vacuum to obtain crude compound, which was purified by recrystallization with methanol to obtain desired product. LCMS: 415 [M+H]⁺; ¹H NMR: (400 MHz, DMSO-d₆): δ 10.71 (br. s., 1H), 8.89 (s, 1H), 8.61 (s, 1H), 7.92-8.02 (m, 1H), 7.77 (br. s., 1H), 7.57 (br. s., 1H), 7.49-7.54 (m, 1H), 5.87 (br. s., 1H), 2.90-2.99 (m, 2H), 2.73 (t, J=6.8 Hz, 2H), 2.20 (br. s., 2H), 1.79-1.99 (m, 6H), 1.59 (d, J=5.3 Hz, 2H).

### Example—S41: Synthesis of 8-cyclopentyl-7-oxo-2-((2-oxo-2,3,4,5-tetrahydro-1H-benzo[b]azepin-7-yl)amino)-7,8-dihydropyrido[2,3-d]pyrimidine-6-carbonitrile (Compound No. 83)

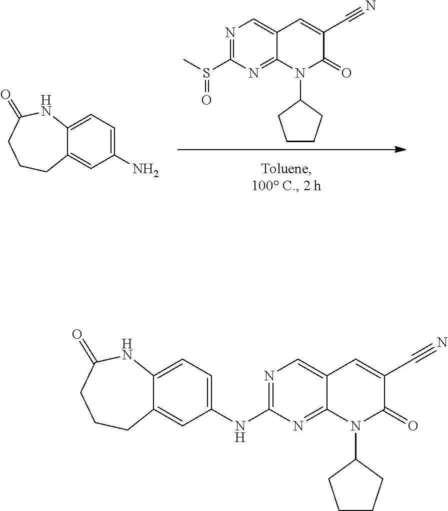 [SMILES: Nc1ccc2c(c1)CCCC(=O)N2 ; CS(=O)c1ncc2cc(C#N)c(=O)n(C3CCCC3)c2n1 ; N#Cc1cc2cnc(Nc3ccc4c(c3)CCCC(=O)N4)nc2n(C2CCCC2)c1=O]

To a stirred solution of 8-cyclobutyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 7-amino-1,3,4,5-tetrahydro-2H-benzo[b]azepin-2-one (64 mg, 0.69 mmol, 1.1 equiv). The resultant reaction mixture was allowed to stir at 100° C. for 2 h. Progress of the reaction was monitored by LCMS. After completion of the reaction, solid observed were filtered and dried under vacuum to obtain crude compound, which was purified by recrystallization with methanol to obtain desired product. LCMS: 415 [M+H]⁺; ¹H NMR: (400 MHz, DMSO-d₆): δ 10.55 (br. s., 1H), 9.48 (br. s., 1H), 8.83 (br. s., 1H), 8.57 (br. s., 1H), 7.71 (br. s., 1H), 7.42 (br. s., 1H), 7.18 (br. s., 1H), 6.96 (d, J=8.3 Hz, 1H), 5.81 (br. s., 1H), 2.67 (br. s., 2H), 2.16 (br. s., 6H), 1.89 (br. s., 2H), 1.80 (br. s., 2H), 1.56 (br. s., 2H)

### Example—S42: Synthesis of 8-cyclopentyl-2-((7-fluoro-1,2,3,4-tetrahydroisoquinolin-6-yl)amino)-7-oxo-7,8-dihydropyrido[2,3-d]pyrimidine-6-carbonitrile (Compound No. 84)

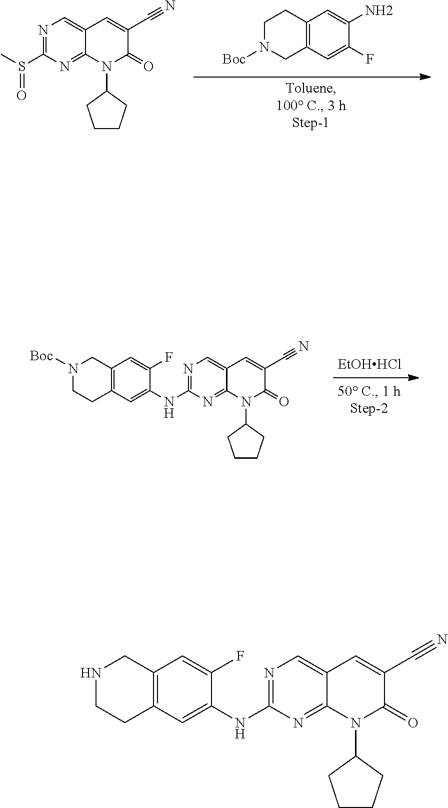

Step—1: Synthesis of tert-butyl 6-((6-cyano-8-cyclopentyl-7-oxo-7,8-dihydropyrido[2,3-d]pyrimidin-2-yl)amino)-7-fluoro-3,4-dihydroisoquinoline-2(1H)-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added tert-butyl 6-amino-7-fluoro-3,4-dihydroisoquinoline-2(1H)-carboxylate (96 mg, 0.36 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain crude compound, which was purified by recrystallization with methanol to obtain desired product. LCMS: 505 [M+H]⁺

Step—2: Synthesis of 8-cyclopentyl-2-((7-fluoro-1,2,3,4-tetrahydroisoquinolin-6-yl)amino)-7-oxo-7,8-dihydropyrido[2,3-d]pyrimidine-6-carbonitrile: A solution of tert-butyl 6-((6-cyano-8-cyclopentyl-7-oxo-7,8-dihydropyrido[2,3-d]pyrimidin-2-yl)amino)-7-fluoro-3,4-dihydroisoquinoline-2(1H)-carboxylate (35 mg, 0.06 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain residue, which was dried under lyophilizer to obtain desired product. LCMS: 405 [M+H]⁺; ¹H NMR: (400 MHz, DMSO-d₆): 10.24 (br. s., 1H), 9.36 (br. s., 1H), 8.82 (br. s., 1H), 8.59 (s, 1H), 7.48 (d, J=5.7 Hz, 1H), 7.24 (d, J=11.4 Hz, 1H), 5.69 (br. s., 1H), 4.26 (br. s., 2H), 3.39 (br. s., 2H), 2.96 (t, J=5.9 Hz, 2H), 2.12 (br. s., 2H), 1.71 (br. s., 4H), 1.50 (br. s., 2H).

### Example—S43: Synthesis of 8-(cyclopropylmethyl)-7-oxo-2-((1,2,3,4-tetrahydroisoquinolin-6-yl)amino)-7,8-dihydropyrido[2,3-d]pyrimidine-6-carbonitrile (Compound No. 85)

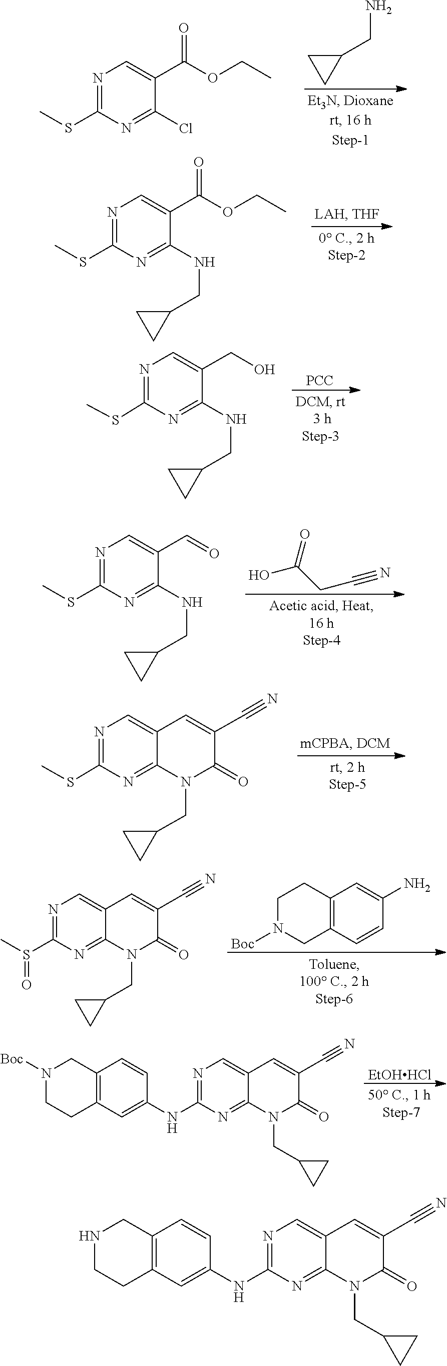

Step—1: Synthesis of ethyl 4-((cyclopropylmethyl)amino)-2-(methylthio)pyrimidine-5-carboxylate: To a stirred solution of ethyl 4-chloro-2-(methylthio)pyrimidine-5-carboxylate (3000 mg, 21.55 mmol, 1 equiv) in Dioxane (40 mL), was added Et₃N (3.6 mL, 25.86 mmol, 2.0 equiv) and cyclopropylmethanamine (1103 mg, 15.51 mmol, 1.2 equiv) at room temperature. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain desired product. LCMS: 268 [M+H]⁺

Step—2: Synthesis of (4-((cyclopropylmethyl)amino)-2-(methylthio)pyrimidin-5-yl)methanol: To a stirred solution of ethyl 4-((cyclopropylmethyl)amino)-2-(methylthio)pyrimidine-5-carboxylate (2000 mg, 7.49 mmol, 1.0 equiv) in THF (20 mL) was added LAH (570 mg, 14.98 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium hydroxide (20 mL) at 0° C. and extracted with ethyl acetate (150 mL×2). Organic layer was washed with water (100 mL) and brine solution (100 mL). Organic layer was dried over anhydrous sodium sulphate and concentrated under reduced pressure to obtain (4-((cyclopropylmethyl)amino)-2-(methylthio)pyrimidin-5-yl)methanol. LCMS: 226 [M+H]⁺

Step—3: Synthesis of 4-((cyclopropylmethyl)amino)-2-(methylthio)pyrimidine-5-carbaldehyde: To a stirred solution of (4-((cyclopropylmethyl)amino)-2-(methylthio)pyrimidin-5-yl)methanol (1200 mg, 5.33 mmol, 1 equiv) in DCM (30 mL), was added PCC (1466 mg, 5.33 mmol, 1 equiv) at RT. The reaction mixture was then allowed to stir at RT for 3 h. Progress of the reaction was monitored by TLC and LCMS. After completion of the reaction, the reaction mixture was passed through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain 4-((cyclopropylmethyl)amino)-2-(methylthio)pyrimidine-5-carbaldehyde. LCMS: 224 [M+H]⁺

Step—4: Synthesis of 8-(cyclopropylmethyl)-2-(methylthio)-7-oxo-7,8-dihydropyrido[2,3-d]pyrimidine-6-carbonitrile: To a stirred solution of 4-((cyclopropylmethyl)amino)-2-(methylthio)pyrimidine-5-carbaldehyde (800 mg, 3.59 mmol, 1 equiv) in Acetic acid (10 mL), was added Cyanoacetic acid (396 mg, 4.66 mmol, 1.3 equiv) and Benzyl amine (0.046 mL, 0.43 mmol, 0.12 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (50 mL) and brine solution (50 mL). Organic layer was dried over anhydrous sodium sulphate and concentrated under reduced pressure to obtain crude compound, which was purified by normal phase combi-flash to obtain desired product. LCMS: 273 [M+H]⁺

Step—5: Synthesis of 8-(cyclopropylmethyl)-2-(methylsulfinyl)-7-oxo-7,8-dihydropyrido[2,3-d]pyrimidine-6-carbonitrile: To a stirred solution of 8-(cyclopropylmethyl)-2-(methylthio)-7-oxo-7,8-dihydropyrido[2,3-d]pyrimidine-6-carbonitrile (200 mg, 0.73 mmol, 1 equiv) in DCM (5 mL), was added m-CPBA (164 mg, 0.95 mmol, 1.3 equiv) at RT. Then the reaction mixture was allowed to stir for 2 h. Progress of the reaction was monitored by LCMS. After completion of the reaction, the reaction mixture was diluted with DCM (50 mL) and washed with saturated solution of NaHCO₃, water (50 mL), brine solution (50 mL). Organic layer was dried over anhydrous sodium sulphate and concentrated under reduced pressure to obtain desired product. LCMS: 289 [M+H]⁺

Step—6: Synthesis of tert-butyl 6-((6-cyano-8-(cyclopropylmethyl)-7-oxo-7,8-dihydropyrido[2,3-d]pyrimidin-2-yl)amino)-3,4-dihydroisoquinoline-2(1H)-carboxylate: To a stirred solution of 8-(cyclopropylmethyl)-2-(methylsulfinyl)-7-oxo-7,8-dihydropyrido[2,3-d]pyrimidine-6-carbonitrile (200 mg, 0.69 mmol, 1 equiv) in toluene (8 mL), was added tert-butyl 6-amino-3,4-dihydroisoquinoline-2(1H)-carboxylate (170 mg, 0.69 mmol, 1.0 equiv). The resultant reaction mixture was allowed to stir at 100° C. for 2 h. Progress of the reaction was monitored by LCMS. After completion of the reaction, solid observed was filtered and dried under vacuum to obtain crude compound, which was purified by recrystallization with methanol to obtain desired product. LCMS: 473 [M+H]⁺

Step—7: Synthesis of 8-(cyclopropylmethyl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-(cyclopropylmethyl)-7-oxo-7,8-dihydropyrido[2,3-d]pyrimidin-2-yl)amino)-3,4-dihydroisoquinoline-2(1H)-carboxylate (30 mg, 0.063 mmol, 1 eq) in 1.25 M HCl in ethanol (5 mL) was allowed to stir for 1 h at 50° C. Progress of the reaction was monitored by LCMS. After completion of the reaction, reaction mixture was cooled to room temperature which results in the formation of precipitates which were filtered and washed with ethanol to obtain desired product. LCMS: 375 [M+H]⁺; ¹H NMR: (400 MHz, DMSO-d₆): δ 10.70 (br. s., 1H), 9.21 (br. s., 2H), 8.89 (s, 1H), 8.65 (s, 1H), 7.82 (br. s., 1H), 7.57 (d, J=9.2 Hz, 1H), 7.23 (d, J=8.3 Hz, 1H), 4.25 (br. s., 2H), 4.18 (d, J=7.0 Hz, 2H), 3.39 (br. s., 2H), 3.03 (br. s., 2H), 1.36 (br. s., 1H), 0.47 (d, J=8.8 Hz, 4H).

### Example—S44: Synthesis of 7-oxo-8-(pentan-3-yl)-2-((1,2,3,4-tetrahydroisoquinolin-6-yl)amino)-7,8-dihydropyrido[2,3-d]pyrimidine-6-carbonitrile (Compound No. 86)

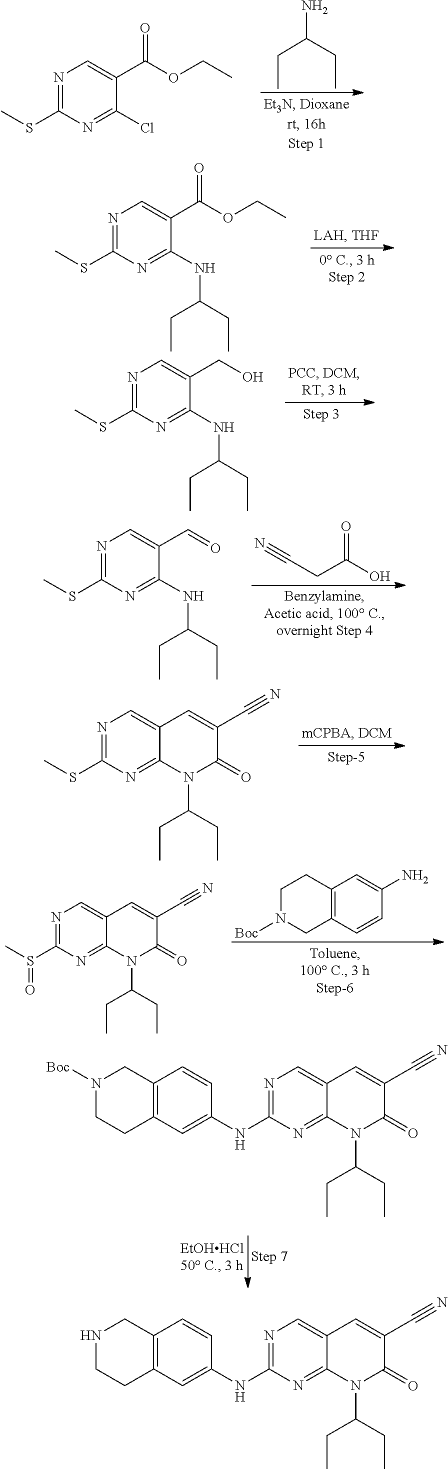

Step—1: Synthesis of ethyl 2-(methylthio)-4-(pentan-3-ylamino)pyrimidine-5-carboxylate: To a stirred solution of ethyl 4-chloro-2-(methylthio)pyrimidine-5-carboxylate (2000 mg, 8.58 mmol, 1 equiv) in Dioxane (20 mL), was added Et₃N (3.6 mL, 10.3 mmol, 1.2 equiv) and pentan-3-amine (896 mg, 10.3 mmol, 1.2 equiv) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain desired product. LCMS: 284 [M+H]⁺

Step—2: Synthesis of (2-(methylthio)-4-(pentan-3-ylamino)pyrimidin-5-yl)methanol: To a stirred solution of ethyl 2-(methylthio)-4-(pentan-3-ylamino)pyrimidine-5-carboxylate (2000 mg, 7.4 mmol, 1 equiv) in THF (50 mL), was added LAH (537 mg, 14.8 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium hydroxide (100 mL) at 0° C. and extracted with ethyl acetate (150 mL×2). Organic layer was washed with water (100 mL) and brine solution (100 mL). Organic layer was dried over anhydrous sodium sulphate and concentrated under reduced pressure to obtain desired product. LCMS: 242 [M+H]⁺

Step—3: Synthesis of 2-(methylthio)-4-(pentan-3-ylamino)pyrimidine-5-carbaldehyde: To a stirred solution of (2-(methylthio)-4-(pentan-3-ylamino) pyrimidin-5-yl)methanol (1500 mg, 6.22 mmol, 1 equiv) in DCM (30 mL), was added PCC (1344 mg, 6.22 mmol, 1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain desired product. LCMS: 240 [M+H]⁺

Step—4: Synthesis of 2-(methylthio)-7-oxo-8-(pentan-3-yl)-7,8-dihydropyrido[2,3-d]pyrimidine-6-carbonitrile: To a stirred solution of 2-(methylthio)-4-(pentan-3-ylamino)pyrimidine-5-carbaldehyde (1100 mg, 4.6 mmol, 1 equiv) in Acetic acid (15 mL), was added Cyanoacetic acid (470 mg, 5.5 mmol, 1.2 equiv) and Benzyl amine (0.1 mL, 0.46, 0.1 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain desired product. LCMS: 289 [M+H]⁺

Step—5: Synthesis of 2-(methylsulfinyl)-7-oxo-8-(pentan-3-yl)-7,8-dihydropyrido[2,3-d]pyrimidine-6-carbonitrile: To a stirred solution of 2-(methylthio)-7-oxo-8-(pentan-3-yl)-7,8-dihydropyrido[2,3-d]pyrimidine-6-carbonitrile (200 mg, 0.69 mmol, 1 equiv) in DCM (5 mL), was added m-CPBA (167 mg, 0.76 mmol, 1.4 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain desired product. LCMS: 305 [M+H]⁺

Step—6: Synthesis of tert-butyl 6-((6-cyano-7-oxo-8-(pentan-3-yl)-7,8-dihydropyrido[2,3-d]pyrimidin-2-yl)amino)-3,4-dihydroisoquinoline-2(1H)-carboxylate: To a stirred solution of 2-(methylsulfinyl)-7-oxo-8-(pentan-3-yl)-7,8-dihydropyrido[2,3-d]pyrimidine-6-carbonitrile (100 mg, 0.33 mmol, 1 equiv) in toluene (5 mL), was added tert-butyl 6-amino-3,4-dihydroisoquinoline-2(1H)-carboxylate (82 mg, 0.36 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain crude compound, which was purified by recrystallization with methanol to obtain desired product. LCMS: 489 [M+H]⁺

Step—7: Synthesis of 7-oxo-8-(pentan-3-yl)-2-((1,2,3,4-tetrahydroisoquinolin-6-yl)amino)-7,8-dihydropyrido[2,3-d]pyrimidine-6-carbonitrile: A solution of tert-butyl 6-((6-cyano-7-oxo-8-(pentan-3-yl)-7,8-dihydropyrido[2,3-d]pyrimidin-2-yl)amino)-3,4-dihydroisoquinoline-2(1H)-carboxylate (50 mg, 0.1 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain residue, which was dried under lyophilizer to obtain desired product. LCMS: 389 [M+H]⁺; ¹H NMR: (400 MHz, DMSO-d₆): 10.66 (br. s., 1H), 9.36 (br. s., 1H), 8.81-8.97 (m, 1H), 8.49-8.67 (m, 1H), 7.67 (d, J=15.8 Hz, 1H), 7.57 (d, J=7.5 Hz, 1H), 7.24 (d, J=7.9 Hz, 1H), 5.41 (br. s., 1H), 4.24 (br. s., 2H), 3.39 (br. s., 2H), 3.01 (br. s., 2H), 2.03-2.25 (m, 2H), 1.74-1.99 (m, 2H), 0.58-0.86 (m, 6H).

### Example—S45: Synthesis of 8-((1r,4r)-4-methylcyclohexyl)-2-((4-(4-methylpiperazin-1-yl)phenyl)amino)-7-oxo-7,8-dihydropyrido[2,3-d]pyrimidine-6-carbonitrile (Compound No. 1)

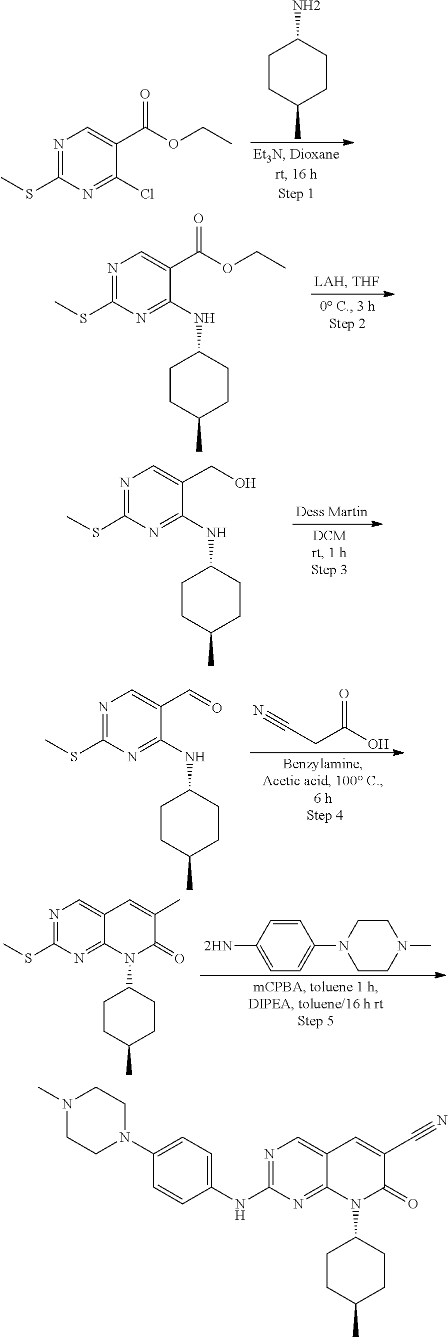

Step—1: Synthesis of ethyl 4-(((1r,4r)-4-methylcyclohexyl)amino)-2-(methylthio)pyrimidine-5-carboxylate: To a stirred solution of ethyl 4-chloro-2-(methylthio)pyrimidine-5-carboxylate (1 g, 4.3 mmol, 1 equiv) in Dioxane (10 mL), was added Et₃N (1.2 mL, 8.6 mmol, 2 equiv) and (1r,4r)-4-methylcyclohexan-1-amine (0.585 g, 5.17 mmol, 1.2 equiv) at room temperature. The resultant reaction mixture was stirred at room temperature for 16 h. Progress of the reaction was monitored by TLC and LCMS. After completion of the reaction, the reaction mixture was diluted with water (30 mL) and extracted with ethyl acetate (100 mL×2). Organic layer was washed with water (100 mL) and brine solution (100 mL). Organic layer was dried over anhydrous sodium sulphate and concentrated under reduced pressure to obtain desired product. LCMS: 310 [M+H]⁺

Step—2: Synthesis of (4-(((1r,4r)-4-methylcyclohexyl)amino)-2-(methylthio)pyrimidin-5-yl)methanol: To a stirred solution of ethyl 4-(((1r, 4r)-4-methylcyclohexyl) amino)-2-(methylthio) pyrimidine-5-carboxylate (1.3 g, 4.20 mmol, 1 equiv) in THF (30 mL), was added LAH (0.558 g, 16.8 mmol, 4 equiv) at 0° C. The reaction mixture was allowed to stir at 0° C. for 3 h. Progress of the reaction was monitored by TLC and LCMS. After completion of the reaction, the reaction mixture was quenched with saturated solution of sodium sulphate at 0° C. and extracted with ethyl acetate (100 mL×3). Organic layer was washed with water (100 mL) and brine solution (100 mL). Organic layer was dried over anhydrous sodium sulphate and concentrated under reduced pressure to obtain the desired product. LCMS: 268.1 [M+H]⁺

Step—3: Synthesis of 4-(((1r,4r)-4-methylcyclohexyl)amino)-2-(methylthio)pyrimidine-5-carbaldehyde: To a stirred solution of (4-(((1r, 4r)-4-methylcyclohexyl) amino)-2-(methylthio) pyrimidin-5-yl) methanol (1.1 g, 4.11 mmol, 1 equiv) in DCM (11 mL), was added Dess-Martin periodinane (0.558 mg, 14.70 mmol, 1.8 equiv) at 0° C. The reaction mixture was allowed to stirr at RT for 1 h. Progress of the reaction was monitored by TLC and NMR. After completion of the reaction, the reaction mixture was quenched with the mixture of saturated solution of sodium thiosulphate: saturated solution of sodium bicarbonate (1: 1, 100 mL), and extracted with ethyl acetate (150 mL×2). Organic layer was washed with water (100 mL) and brine solution (100 mL). Organic layer was dried over anhydrous sodium sulphate and concentrated under reduced pressure to obtain desired product. LCMS: 266 [M+H]⁺

Step—4: Synthesis of 8-((1r,4r)-4-methylcyclohexyl)-2-(methylthio)-7-oxo-7,8-dihydropyrido[2,3-d]pyrimidine-6-carbonitrile: To a stirred solution of 4-(((1r, 4r)-4-methylcyclohexyl) amino)-2-(methylthio) pyrimidine-5-carbaldehyde (500 mg, 1.886 mmol, 1 eqiv) in Acetic acid (10 mL), was added cyano acetic acid (192.3 mg, 2.263 mmol, 1.2 eqiv) and benzyl amine (0.02 mL, 0.188, 0.1 eqiv). The reaction mixture was heated to 100° C. for 6 h under reflux condition. Progress of the reaction was monitored by TLC and LCMS. After completion of the reaction, the reaction mixture was diluted with water (30 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combiflsh. LCMS: 315 [M+H]⁺

Step—5: Synthesis of 8-((1r,4r)-4-methylcyclohexyl)-2-((4-(4-methylpiperazin-1-yl)phenyl)amino)-7-oxo-7,8-dihydropyrido[2,3-d]pyrimidine-6-carbonitrile: To a stirred solution of 8-((1r, 4r)-4-methylcyclohexyl)-2-(methylthio)-7-oxo-7, 8-dihydropyrido [2, 3-d]pyrimidine-6-carbonitrile (110 mg, 0.349 mmol, 1 equiv) in Toluene (5 mL), was added m-CPBA (120 mg, 0.698 mmol, 2 equiv) at RT and stirred for 30 min. Progress of the reaction was monitored by TLC to confirm the formation of sulfonyl. To the above mixture 4-(4-methylpiperazin-1-yl) aniline (80 mg, 0.419 mmol, 1.2 equiv) was added, followed by the addition of DIPEA (0.3 mL, 1.74 mmol, 5 equiv). The resultant reaction mixture was stirred at RT for 1 h. Progress of the reaction was monitored by LCMS. Solid observed was filtered, washed with pentane (50 mL) and dried under vacuum to obtain solid crude compound, which was purified by reverse phase HPLC to afford desired product. LCMS: 458.3 [M+H]⁺; ¹H NMR (400 MHz, Methanol-d4) δ 8.70 (s, 1H), 8.32 (s, 1H), 7.56 (s, 2H), 7.05 (d, J=8.6 Hz, 2H), 4.60 (s, 1H), 3.39-3.32 (m, 4H), 3.14 (s, 4H), 2.74 (s, 3H), 2.63 (s, 2H), 1.85 (s, 2H), 1.62 (s, 2H), 1.29 (s, 1H), 1.09 (s, 2H), 0.96 (s, 3H).

### Example—S46: Synthesis of 3-((6-cyano-8-((1r,4r)-4-methylcyclohexyl)-7-oxo-7,8-dihydropyrido[2,3-d]pyrimidin-2-yl)amino)benzenesulfonamide (Compound No. 2)

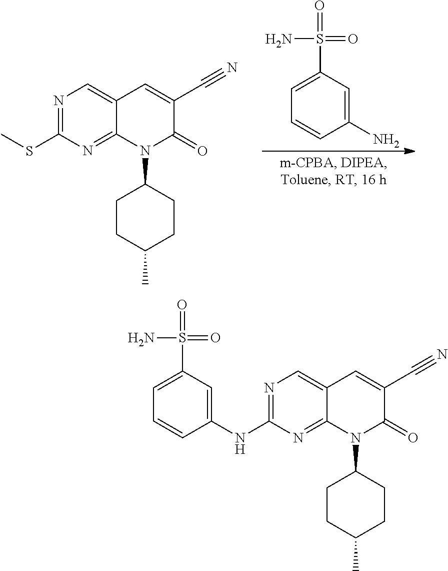

To a stirred solution of 8-((1r,4r)-4-methylcyclohexyl)-2-(methylthio)-7-oxo-7,8-dihydropyrido[2,3-d]pyrimidine-6-carbonitrile (100 mg, 0.318 mmol, 1 equiv) in Toluene (5 mL), was added m-CPBA (110 mg, 0.636 mmol, 2 equiv) at room temperature. Reaction mixture was stirred at room temperature for 30 min. Progress of the reaction was monitored by TLC. To the above mixture, 3-aminobenzenesulfonamide (65.63 mg, 0.381 mmol) was added followed by the addition of DIPEA (0.3 mL, 1.59 mmol). The resultant reaction mixture was stirred at room temperature for 16 h. Progress of the reaction was monitored by LCMS. Solid observed was filtered and washed with pentane (50 mL) and dried under vacuum to obtain crude compound, which was purified by reverse phase HPLC to afford 09 mg. off white solid. LCMS: 439 [M+H]⁺; ¹H NMR (400 MHz, DMSO-d₆) δ 10.70 (s, 1H), 8.9 (s, 1H), 8.6 (s, 2H), 8.05 (s, 1H), 7.6 (m, 2H), 7.4 (s, 2H), 5.3 (s, 1H), 1.80 (m, 2H), 1.6 (m, 2H), 1.4 (m, 2H), 1.3 (s, 1H), 1.1 (m, 2H), 0.96 (q, 3H).

### Example—S47: Synthesis of 3-((6-cyano-8-cyclopentyl-7-oxo-7,8-dihydropyrido[2,3-d]pyrimidin-2-yl)amino)benzenesulfonamide (Compound No. 3)

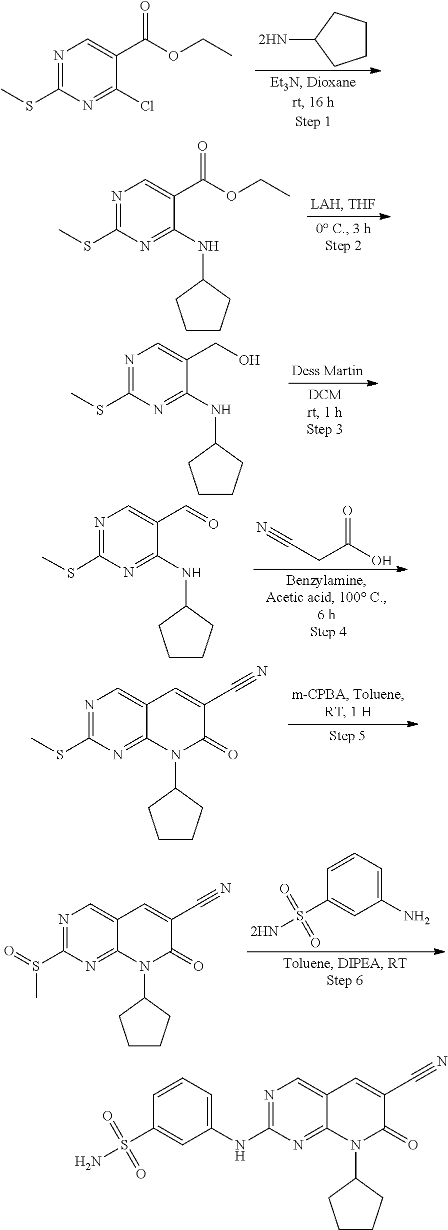

Step—1: Synthesis of ethyl 4-(cyclopentylamino)-2-(methylthio)pyrimidine-5-carboxylate: To a stirred solution of ethyl 4-chloro-2-(methylthio)pyrimidine-5-carboxylate (5 g, 21.55 mmol, 1.0 equiv) in dioxane (50 mL), was added triethylamine (6.05 mL, 43.1 mmol, 2.0 equiv) and cyclopentanamine (2.198 g, 25.82 mmol, 1.2 equiv) at room temperature. Stirred the reaction mixture for 16 h at room temperature. Progress of the reaction was monitored by TLC and LCMS. After completion of the reaction, the reaction mixture was diluted with water (100 mL) and extracted with ethyl acetate (100 mL×2), organic layer was washed with water (100 mL) and brine solution (100 mL), dried over anhydrous sodium sulphate. Concentrated under reduced pressure to obtain desired product. LCMS: 282 [M+H]⁺

Step—2: Synthesis of ethyl (4-(cyclopentylamino)-2-(methylthio)pyrimidin-5-yl)methanol: To a stirred solution of ethyl 4-(cyclopentylamino)-2-(methylthio)pyrimidine-5-carboxylate (7 g, 24.91 mmol, 1.0 equiv) in THF (100 mL), was added portion wise LAH (2.836 g, 74.73 mmol) at 0° C. The reaction mixture was allowed to stir at room temperature for 3 h. Progress of the reaction was monitored by TLC and LCMS. After completion of the reaction, the reaction mixture was quenched with saturated solution of sodium sulphate dropwise at 0° C. Obtained residue was filtered through celite bed. Filtrate was extracted with ethyl acetate (100 mL×2). The combined organic layer was washed with water (100 mL) and brine solution (100 mL), dried over anhydrous sodium sulphate and concentrated under reduced pressure to obtain desired product. LCMS: 240 [M+H]⁺

Step—3: Synthesis of 4-(cyclopentylamino)-2-(methylthio)pyrimidine-5-carbaldehyde: To a stirred solution of ethyl (4-(cyclopentylamino)-2-(methylthio)pyrimidin-5-yl)methanol (5 g, 20.92 mmol, 1.0 equiv) in DCM (50 mL), was added pyridinium chlorochromate (8.995 g, 41.84 mmol, 2.0 equiv) at 0° C. The reaction mixture was allowed to stirred at room temperature for 1 h. Progress of the reaction was monitored by TLC and LCMS. After completion of the reaction, workup done by filtration of reaction mass through celite pad by and celite bed was washed by DCM (50 mL×2) filtrate was diluted with water (100 mL), and extracted with DCM (100 mL×2). The combined organic layer was washed with sodium bicarbonate solution (100 mL) and brine solution (100 mL), dried over anhydrous sodium sulphate and concentrated under reduced pressure to obtain desired product. LCMS: 238 [M+H]⁺

Step—4: Synthesis of 8-cyclopentyl-2-(methylthio)-7-oxo-7,8-dihydropyrido[2,3-d]pyrimidine-6-carbonitrile: To a stirred solution of 4-(cyclopentylamino)-2-(methylthio)pyrimidine-5-carbaldehyde (5 g, 21.09 mmol, 1.0 equiv.) in Acetic acid (50 mL), was added cyano acetic acid (2.151 g, 25.31 mmol, 1.2 equiv) and Benzyl amine (0.250 g, 2.109 mmol, 0.1 equiv). The reaction mixture was allowed to stir at 100° C. for 6 h. Progress of the reaction was monitored by TLC and LCMS. After completion of the reaction, the reaction mixture was diluted with water (50 mL) and extracted with ethyl acetate (50 mL×2). The combined organic layer was washed with water (50×2 mL) and brine solution (50 mL). Organic layer was dried over anhydrous sodium sulphate and concentrated under reduced pressure to obtain crude compound, which was purified by normal phase combi-flash to obtain desired product. LCMS: 287 [M+H]⁺

Step—5: Synthesis of 8-cyclopentyl-2-(methylsulfinyl)-7-oxo-7,8-dihydropyrido[2,3-d]pyrimidine-6-carbonitrile: To a stirred solution of obtain 8-cyclopentyl-2-(methylthio)-7-oxo-7,8-dihydropyrido[2,3-d]pyrimidine-6-carbonitrile (500 mg, 1.74 mmol, 1 equiv) in toluene (5 mL) was added m-CPBA (330 mg, 1.92 mmol, 1.4 equiv) at room temperature. Reaction mass was stirred at room temperature for 1 h. Progress of the reaction was monitored by TLC and LCMS. After completion of the reaction, the reaction mixture was diluted with sodium bicarbonate solution (10 mL) and extracted with ethyl acetate (15 mL×2). The combined organic layer was washed with water (10 mL) and sodium bicarbonate solution (50 mL) brine solution (50 mL), dried over anhydrous sodium sulphate and concentrated under reduced pressure to obtain desired product. LCMS: 303 [M+H]⁺; ¹H NMR: (400 MHz, DMSO-d6): δ 9.28 (s, 1H), 8.92 (s, 1H), 5.78-5.94 (m, 1H), 2.95 (s, 3H), 2.14-2.26 (m, 2H), 2.01-2.14 (m, 2H), 1.82-1.93 (m, 2H), 1.55-1.69 (m, 2H).

Step—6: Synthesis of 3-((6-cyano-8-cyclopentyl-7-oxo-7,8-dihydropyrido[2,3-d]pyrimidin-2-yl)amino)benzenesulfonamide: To a stirred solution of 8-cyclopentyl-2-(methylsulfonyl)-7-oxo-7,8-dihydropyrido[2,3-d]pyrimidine-6-carbonitrile (350 mg, 1.1 mmol, 1.0 equiv.) and 3-aminobenzenesulfonamide (17 mg, 0.990 mmol, 0.9 equiv). The reaction mixture was allowed to stir for 16 h at room temperature. Progress of the reaction was monitored by LCMS. After completion of the reaction, the reaction mixture was diluted with water (30 mL) and extracted with ethyl acetate (50 mL×2). The combined organic layer was washed with water (50 mL), sodium bicarbonate solution (50 ml×2) and brine solution (100 mL), dried over anhydrous sodium sulphate and concentrated under reduced pressure to obtain crude compound, which was purified by reverse phase chromatography to obtain desired product. LCMS: 411 [M+H]⁺; ¹H NMR (400 MHz, DMSO-d₆) δ 10.79 (br. s., 1H), 8.90 (s, 1H), 8.62 (s, 1H), 8.52 (br. s., 1H), 7.74 (br. s., 1H), 7.54-7.59 (m, 2H), 7.35 (s, 2H), 5.88-5.97 (m, 1H), 2.13 (br. s., 2H), 1.89 (d, J=17.10 Hz, 4H), 1.65 (br. s., 2H).

### Example—S48: Synthesis of 3-((6-cyano-8-((1r,4r)-4-methylcyclohexyl)-7-oxo-7,8-dihydropyrido[2,3-d]pyrimidin-2-yl)amino)benzamide (Compound No. 4)

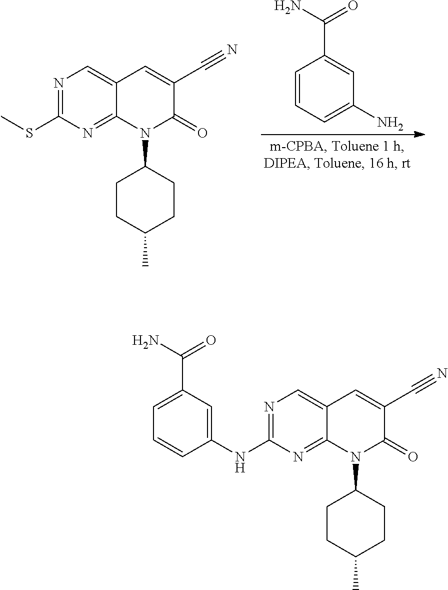 [SMILES: CSc1ncc2cc(C#N)c(=O)n([C@H]3CC[C@H](C)CC3)c2n1 ; NC(=O)c1cccc(N)c1 ; C[C@H]1CC[C@H](n2c(=O)c(C#N)cc3cnc(Nc4cccc(C(N)=O)c4)nc32)CC1]

To a stirred solution of 8-((1r,4r)-4-methylcyclohexyl)-2-(methylthio)-7-oxo-7,8-dihydropyrido[2,3-d]pyrimidine-6-carbonitrile (350 mg, 1.114 mmol) in Toluene 10 ml and 5 ml THF was added a mCPBA 55% in aq. (210 mg, 1.226 mmol) was stirred for 60 min, LCMS confirms the formation of sulfonyl. Solid residue was filtered, organic layer was concentrated under vacuum. To this was added 3-aminobenzamide (70.75 mg, 0.520 mmol) followed by DIPEA (0.31 ml, 1.734 mmol) in Toluene (5 ml) and THF (5 ml). The resultant reaction mixture was stir at rt for 16 h. Progress of the reaction was monitored by LCMS. Solid observed was filtered and washed with pentane (50 mL) and dried under vacuum to obtain crude compound, which was purified by reverse phase purification to afford desired product. LCMS: 403 [M+H]^{+ 1}H NMR (400 MHz, DMSO-d₆) δ 10.7 (bs, 1H), 8.9 (s, 1H), 8.6 (s, 1H), 8.09 (s, 2H), 7.6 (s, 2H), 7.38 (s, 2H), 5.3 (bs, 1H), 1.8 (d, 2H), 1.6 (d, 2H), 1.40 (s, 2H), 1.21 (s, 1H), 1.09 (d, 2H), 0.96 (q, 3H).

### Example—S49: Synthesis of 3-((6-cyano-8-cyclopentyl-7-oxo-7,8-dihydropyrido[2,3-d]pyrimidin-2-yl)amino)benzamide (Compound No. 5)

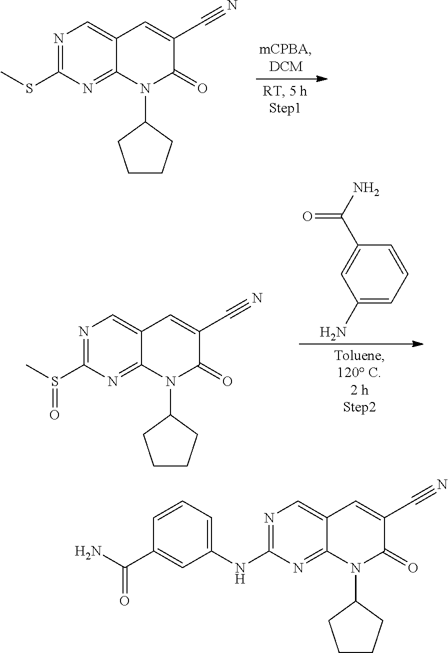

Step—1: Synthesis of 8-cyclopentyl-2-(methylsulfinyl)-7-oxo-7,8-dihydropyrido[2,3-d]pyrimidine-6-carbonitrile: To a stirred solution of 8-cyclopentyl-2-(methylthio)-7-oxo-7,8-dihydropyrido[2,3-d]pyrimidine-6-carbonitrile (200 mg, 0.78 mmol) in DCM (6 mL) was added metachloroperbenzoicacid (173 mg, 1.00 mmol) at rt. Resultant mixture was stirred at room temperature for 6 h. Progress of the reaction was monitored by TLC. After completion of reaction, reaction mixture was diluted with water (10 mL) and extracted with DCM (15 mL×2). The combined organic layer was washed with saturated NaHCO₃ solution (50 mL), dried over anhydrous sodium sulphate and concentrated under reduced pressure to afford desired product. LCMS: [M+H]⁺

Step—2: Synthesis of 3-((6-cyano-8-cyclopentyl-7-oxo-7,8-dihydropyrido[2,3-d]pyrimidin-2-yl)amino)benzamide: To a suspension of 8-cyclopentyl-2-(methylsulfinyl)-7-oxo-7,8-dihydropyrido[2,3-d]pyrimidine-6-carbonitrile (50 mg, 0.16 mmol) in toluene (5 mL) was added 3-aminobenzamide (26 mg, 0.19 mmol) The resultant reaction mixture was stir at 100° C. for 2 h. Progress of the reaction was monitored by LCMS. Product precipitates out, was filtered and triturated from methanol to afford desired product. LCMS: 375 [M+H]⁺; ¹H NMR (400 MHz, DMSO-d₆) δ 10.64 (br. s., 1H), 8.87 (s, 1H), 8.59 (s, 1H), 8.50 (br. s., 1H), 7.94 (br. s., 1H), 7.60-7.71 (m, 2H), 7.36-7.48 (m, 2H), 5.84-5.96 (m, 1H), 2.14 (br. s., 2H), 1.85 (br. s., 4H), 1.62 (br. s., 2H)

### Example—S50: Synthesis of 8-cyclopentyl-2-((3-(hydroxymethyl)-4-(4-methylpiperazin-1-yl)phenyl)amino)-7-oxo-7,8-dihydropyrido[2,3-d]pyrimidine-6-carbonitrile (Compound No. 10)

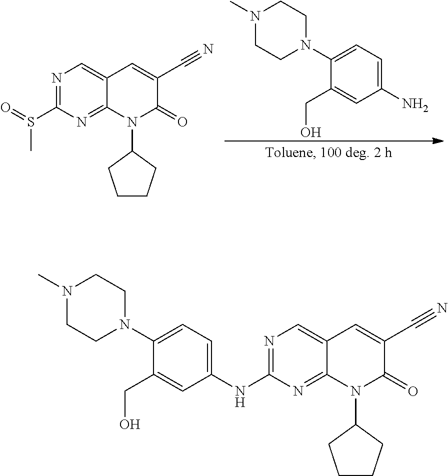 [SMILES: CS(=O)c1ncc2cc(C#N)c(=O)n(C3CCCC3)c2n1 ; CN1CCN(c2ccc(N)cc2CO)CC1 ; CN1CCN(c2ccc(Nc3ncc4cc(C#N)c(=O)n(C5CCCC5)c4n3)cc2CO)CC1]

To a suspension of 8-cyclopentyl-2-(methylsulfinyl)-7-oxo-7,8-dihydropyrido[2,3-d]pyrimidine-6-carbonitrile (50 mg, 0.16 mmol) in toluene (5 mL) was added (5-amino-2-(4-methylpiperazin-1-yl)phenyl)methanol (40 mg, 0.18 mmol). Resultant reaction mixture was stirred at 100° C. for 2 h. Progress of the reaction was monitored by LCMS. Crude product precipitates out was filtered, purified by reverse phase HPLC to afford desired product. LCMS: 460. [M+H]⁺; ¹H NMR (400 MHz, Methanol-d4) δ 8.73 (s, 1H), 8.34 (s, 1H), 7.90 (br. s., 1H), 7.47 (br. s., 1H), 7.17 (d, J=9.21 Hz, 1H), 6.01 (br. s., 1H), 4.74 (s, 2H), 3.03 (br. s., 4H), 2.83 (br. s., 4H), 2.51 (s, 3H), 2.23 (d, J=15.35 Hz, 2H), 1.84-2.00 (m, 4H), 1.67 (br. s., 2H).

### Example—S51: Synthesis of 3-((6-cyano-8-cyclopentyl-7-oxo-7,8-dihydropyrido[2,3-d]pyrimidin-2-yl)amino)-N-(2-(dimethylamino)ethyl)benzamide (Compound No. 11)

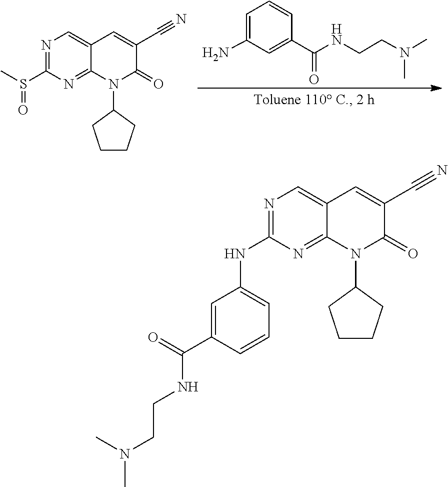 [SMILES: CS(=O)c1ncc2cc(C#N)c(=O)n(C3CCCC3)c2n1 ; CN(C)CCNC(=O)c1cccc(N)c1 ; CN(C)CCNC(=O)c1cccc(Nc2ncc3cc(C#N)c(=O)n(C4CCCC4)c3n2)c1]

To a suspension of 8-cyclopentyl-2-(methylsulfinyl)-7-oxo-7,8-dihydropyrido[2,3-d]pyrimidine-6-carbonitrile (60 mg, 0.2 mmol, 1 equiv) in toluene (4 mL), was added 3-amino-N-(2-(dimethylamino)ethyl)benzamide (45 mg, 0.22 mmol, 1.1 equiv). Resultant reaction mixture was stirred at 110° C. for 2 h. Reaction was monitored by LCMS. After completion of reaction, reaction mass concentrated under reduced pressure. Crude mixture was purified by reverse phase HPLC to afford 6 mg of 3-((6-cyano-8-cyclopentyl-7-oxo-7,8-dihydropyrido[2,3-d]pyrimidin-2-yl)amino)-N-(2-(dimethylamino)ethyl)benzamide. LCMS: 446 [M+H]⁺; ¹H NMR: (400 MHz, Methanol-d4) δ 8.79 (s, 1H), 8.58 (s, 1H), 8.45-8.37 (m, 2H), 7.74 (d, J=7.6 Hz, 1H), 7.59 (d, J=7.6 Hz, 1H), 7.47 (t, J=7.9 Hz, 1H), 6.09-5.98 (m, 1H), 3.67 (t, J=6.2 Hz, 2H), 3.06 (d, J=6.5 Hz, 2H), 2.72 (s, 6H), 2.26 (t, J=9.8 Hz, 2H), 2.02-1.85 (m, 5H), 1.68 (d, J=7.7 Hz, 2H).

### Example—S52: Synthesis of 8-cyclopentyl-7-oxo-2-((4-(2-oxopiperazin-1-yl)phenyl)amino)-7,8-dihydropyrido[2,3-d]pyrimidine-6-carbonitrile (Compound No. 12)

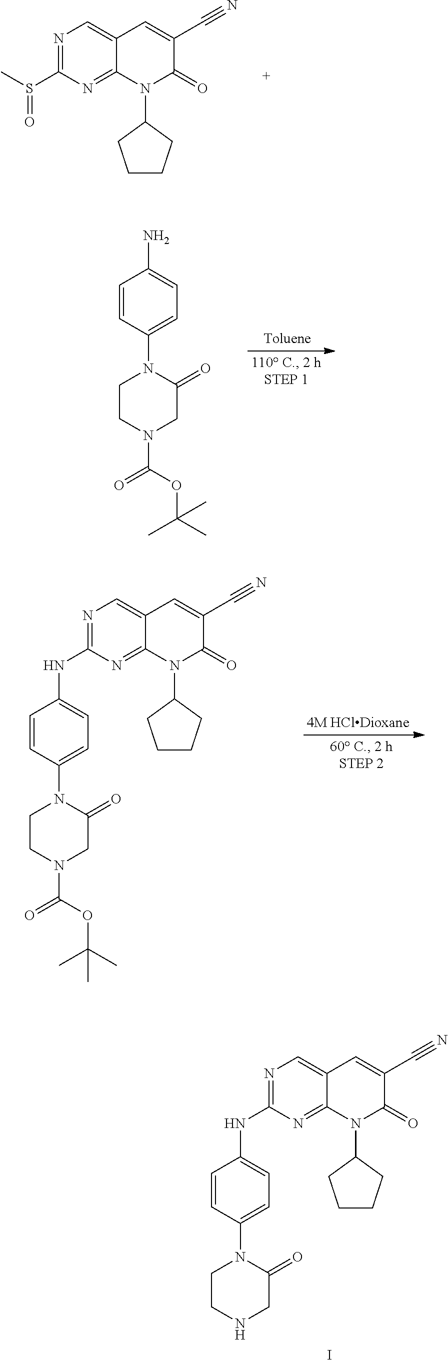

Step—1: Synthesis of tert-butyl 4-(4-((6-cyano-8-cyclopentyl-7-oxo-7,8-dihydropyrido[2,3-d]pyrimidin-2-yl)amino)phenyl)-3-oxopiperazine-1-carboxylate: To a suspension of 8-cyclopentyl-2-(methylsulfinyl)-7-oxo-7,8-dihydropyrido[2,3-d]pyrimidine-6-carbonitrile (60 mg, 0.2 mmol, 1 equiv) in toluene (4 mL), was added tert-butyl 4-(4-aminophenyl)-3-oxopiperazine-1-carboxylate (64 mg, 0.22 mmol, 1.1 equiv). Resultant reaction mixture was stirred at 110° C. for 2 h. Reaction was monitored by LCMS. After completion of reaction, volatiles were removed under reduced pressure. Product was triturated from methanol to afford desired product. LCMS: 530 [M+H]⁺

Step—2: Synthesis of 8-cyclopentyl-7-oxo-2-((4-(2-oxopiperazin-1-yl)phenyl)amino)-7,8-dihydropyrido[2,3-d]pyrimidine-6-carbonitrile: Tert-butyl 4-(4-((6-cyano-8-cyclopentyl-7-oxo-7,8-dihydropyrido[2,3-d]pyrimidin-2-yl)amino)phenyl)-3-oxopiperazine-1-carboxylate (60 mg, 0.113 mmol, 1 equiv) was added to the 4M HCl in dioxane (4 mL). The resultant reaction mixture was stirred at 60° C. for 2 h. Reaction was monitored by LCMS. After completion of reaction, filtered the solid product and dried the solid under vacuum to afford 30 mg of 8-cyclopentyl-7-oxo-2-((4-(2-oxopiperazin-1-yl)phenyl)amino)-7,8-dihydropyrido[2,3-d]pyrimidine-6-carbonitrile hydrochloride. LCMS: 430 [M+H]⁺; ¹H NMR: (400 MHz, Methanol-d4) δ 8.78 (d, J=4.1 Hz, 1H), 8.38 (d, J=4.5 Hz, 1H), 7.82-7.75 (m, 2H), 7.36 (d, J=8.0 Hz, 2H), 5.98 (d, J=10.3 Hz, 1H), 4.00 (dd, J=14.8, 5.0 Hz, 4H), 3.70 (q, J=6.1 Hz, 4H), 2.34-2.24 (m, 2H), 2.02 (s, 2H), 1.67 (d, J=9.0 Hz, 2H).

### Example—S53: Synthesis of 8-cyclopentyl-2-((3-fluoro-4-(piperazin-1-yl)phenyl)amino)-7-oxo-7,8-dihydropyrido[2,3-d]pyrimidine-6-carbonitrile (Compound No. 13)

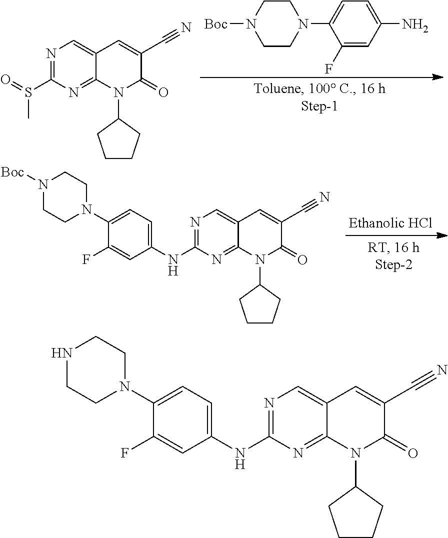

Step—1: Synthesis of tert-butyl 4-(4-((6-cyano-8-cyclopentyl-7-oxo-7,8-dihydropyrido[2,3-d]pyrimidin-2-yl)amino)-2-fluorophenyl)piperazine-1-carboxylate: To a suspension of 8-cyclopentyl-2-(methylsulfinyl)-7-oxo-7,8-dihydropyrido[2,3-d]pyrimidine-6-carbonitrile (75 mg, 0.248 mmol, 1 equiv) in toluene (4 mL), was added tert-butyl 4-(4-amino-2-fluorophenyl)piperazine-1-carboxylate (80.59 mg, 0.273 mmol, 1.1 equiv). Resultant reaction mixture was stirred at 100° C. for 16 h. Reaction was monitored by LCMS. After completion of reaction, volatiles were removed under reduced pressure. Product was triturated from methanol to afford desired product. LCMS: 534.2 [M+H]⁺

Step—2: Synthesis of 8-cyclopentyl-2-((3-fluoro-4-(piperazin-1-yl)phenyl)amino)-7-oxo-7,8-dihydropyrido[2,3-d]pyrimidine-6-carbonitrile: The tert-butyl 4-(4-((6-cyano-8-cyclopentyl-7-oxo-7,8-dihydropyrido[2,3-d]pyrimidin-2-yl)amino)-2-fluorophenyl)piperazine-1-carboxylate (50 mg, 0.09 mmol, 1 equiv) was added to the ethanolic HCL (3 mL). The resultant reaction mixture was stirred at RT for 16 h. Reaction was monitored by LCMS. After completion of reaction, filtered the solid product and dried the solid under vacuum to afford 39 mg of 8-cyclopentyl-2-((3-fluoro-4-(piperazin-1-yl)phenyl)amino)-7-oxo-7,8-dihydropyrido[2,3-d]pyrimidine-6-carbonitrile. LCMS: 434.2 [M+H]⁺; ¹H NMR (400 MHz, DMSO-d₆) δ 10.61 (br. s., 1H), 9.21 (br. s., 1H), 8.85 (s, 1H), 8.59 (s, 1H), 7.70 (d, J=15.35 Hz, 1H), 7.36 (br. s., 1H), 7.04-7.19 (m, 1H), 5.79 (br. s., 1H), 3.22 (br. s., 8H), 2.21 (br. s., 2H), 1.90 (br. s., 2H), 1.79 (br. s., 2H), 1.59 (br. s., 2H).

### Example—S54: Synthesis of 8-cyclopentyl-2-((4-((4-hydroxypiperidin-1-yl)sulfonyl)phenyl)amino)-7-oxo-7,8-dihydropyrido[2,3-d]pyrimidine-6-carbonitrile (Compound No. 14)

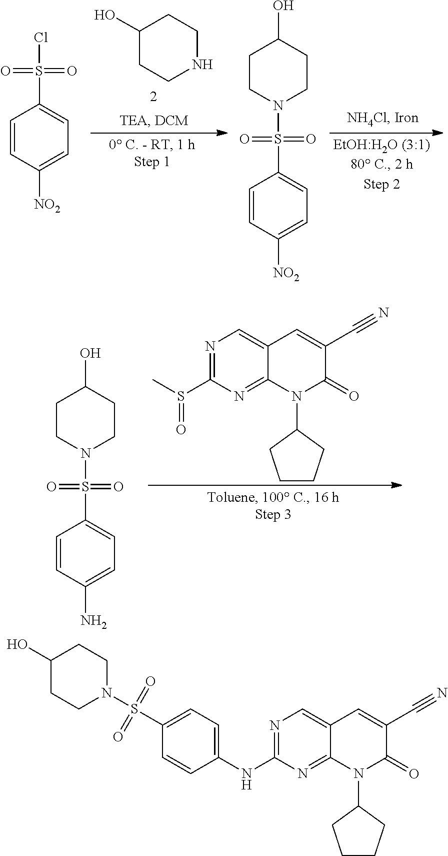

Step—1: Synthesis of 1-((4-nitrophenyl)sulfonyl)piperidin-4-ol: To a stirred solution of piperidin-4-ol (1.1 g, 0.0108 mol, 1.2 equiv) in DCM (20 mL), was dropwise added triethylamine (1.82 g, 0.018 mol, 2 equiv) at 0° C. The resultant reaction mixture was stirred at 0° C. for 10 min. Then 4-nitrobenzenesulfonyl chloride (2 gm, 0.009 mol, 1 equiv) was added and stirred for 1 h at RT. Reaction was monitored by TLC. After completion of reaction, water was added to the reaction mixture and extracted with DCM (20 mL×2). Combined all organic layers and washed with water (20 mL×2) and brine solution (20 mL). The organic layer was dried over anhydrous sodium sulphate and concentrated under reduced pressure to obtain desired product. LCMS: 287 [M+H]⁺

Step—2: Synthesis of 1-((4-aminophenyl)sulfonyl)piperidin-4-ol: To a stirred solution of 1-((4-nitrophenyl)sulfonyl)piperidin-4-ol (2.2 g, 0.0077 mol, 1 equiv) in EtOH (30 mL), was added Iron (4.30 g, 0.077 mol, 10 equiv), ammonium chloride (4.11 g, 0.077 mol, 10 equiv) and water (10 ml). The resultant reaction mixture was heated at 80° C. for 2 h. Reaction mixture was filtered through the celite. The filtrate was concentrated under reduced pressure. Aqueous layer was extracted by the ethyl acetate (30 mL×3). All organic layers was combined and washed by brine solution (20 mL). The organic layer was dried over anhydrous sodium sulphate and concentrated under reduced pressure to obtain desired product: LCMS: 257 [M+H]⁺

Step—3: Synthesis of 8-cyclopentyl-2-((4-((4-hydroxypiperidin-1-yl)sulfonyl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (50 mg, 0.165 mmol, 1 equiv) in toluene (2 mL), was added 1-((4-aminophenyl)sulfonyl)piperidin-4-ol (46.08 mg, 0.18 mmol, 1.1 equiv). The resultant reaction mixture was heated at 100° C. for 16 h. Reaction was monitored by LCMS. After completion of reaction, solid precipitated out, was filtered and purified by reverse phase HPLC to afford desired product. LCMS: 495 [M+H]⁺; ¹H NMR: (400 MHz, Chloroform-d) δ 8.63 (s, 1H), 8.05 (s, 1H), 7.78 (d, J=8.1 Hz, 2H), 7.66 (d, J=8.5 Hz, 2H), 5.94-5.69 (m, 1H), 3.58 (d, J=18.6 Hz, 2H), 3.2-3.4 (m, 8H), 2.84-2.70 (m, 2H), 2.23 (d, J=9.6 Hz, 2H), 2.08-1.95 (m, 2H), 1.84 (d, J=13.0 Hz, 4H).

### Example—S55: Synthesis of 8-cyclopentyl-2-((4-((4-methylpiperazin-1-yl)sulfonyl)phenyl)amino)-7-oxo-7,8-dihydropyrido[2,3-d]pyrimidine-6-carbonitrile (Compound No. 15)

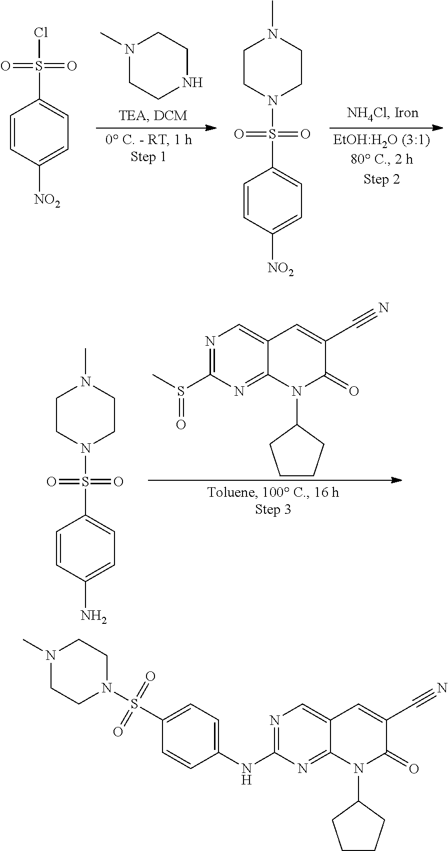

Step—1: Synthesis of 1-methyl-4-((4-nitrophenyl)sulfonyl)piperazine: To a stirred solution of 1-methylpiperazine (1.2 g, 0.0108 mol, 1.2 equiv) in DCM (20 mL), was dropwise added triethylamine (1.82 g, 0.018 mol, 2 equiv) at 0° C. The resultant reaction mixture was stirred at 0° C. for 10 min. Then 4-nitrobenzenesulfonyl chloride (2 gm, 0.009 mol, 1 equiv) was added and stirred for 1 h at RT. Reaction was monitored by TLC. After completion of reaction, water was added to the reaction mixture and extracted with DCM (20 mL×2). Combined organic layers were washed with water (20 mL×2) and brine solution (20 mL). The organic layer was dried over anhydrous sodium sulphate and concentrated under reduced pressure to obtain desired product. LCMS: 286 [M+H]⁺

Step—2: Synthesis of 4-((4-methylpiperazin-1-yl)sulfonyl)aniline: To a stirred solution of 1-methyl-4-((4-nitrophenyl)sulfonyl)piperazine (2.4 g, 0.0084 mol, 1 equiv) in EtOH (30 mL), was added Iron (4.70 g, 0.084 mol, 10 equiv), ammonium chloride (4.5 g, 0.084 mol, 10 equiv) and water (10 ml). The resultant reaction mixture was heated at 80° C. for 2 h. Reaction mixture was filtered through the celite. The filtrate was concentrated under reduced pressure. Aqueous layer was extracted by the ethyl acetate (30 mL×3). All organic layers was combined and washed by brine solution (20 mL). The organic layer was dried over anhydrous sodium sulphate and concentrated under reduced pressure to obtain desired product. LCMS: 256 [M+H]⁺

Step—3: Synthesis of 8-cyclopentyl-2-((4-((4-methylpiperazin-1-yl)sulfonyl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (50 mg, 0.165 mmol, 1 equiv) in toluene (2 mL), was added 4-((4-methylpiperazin-1-yl)sulfonyl)aniline (49.09 mg, 0.18 mmol, 1.1 equiv). The resultant reaction mixture was heated at 100° C. for 16 h. Reaction was monitored by LCMS. After completion of reaction, solid precipitated out was filtered and purified by reverse phase HPLC to afford desired product. LCMS: 494 [M+H]⁺; ¹H NMR: (400 MHz, Chloroform-d) δ 8.68 (s, 1H), 8.05 (s, 1H), 7.79 (q, J=8.8 Hz, 4H), 7.70 (s, 1H), 5.87 (p, J=9.2, 8.7 Hz, 1H), 3.07 (s, 4H), 2.50 (t, J=5.2 Hz, 4H), 2.28 (s, 4H), 2.09 (d, J=5.9 Hz, 2H), 1.92 (q, J=10.3, 9.5 Hz, 2H), 1.75-1.65 (m, 2H).

### Example—S56: Synthesis of 8-cyclopentyl-2-((3-(4-methylpiperazine-1-carbonyl)phenyl)amino)-7-oxo-7,8-dihydropyrido[2,3-d]pyrimidine-6-carbonitrile (Compound No. 16)

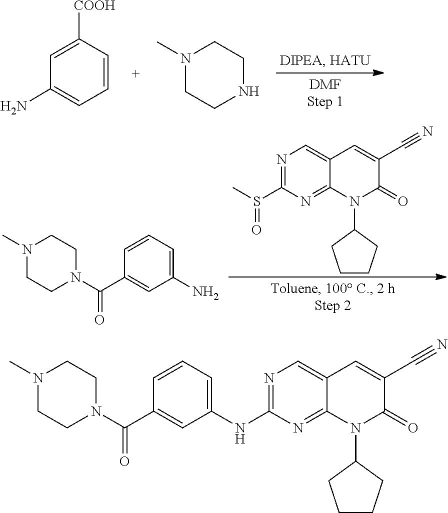

Step—1: Synthesis of (3-aminophenyl)(4-methylpiperazin-1-yl)methanone: To a stirred solution of 1-methylpiperazine (730 mg, 0.0073 mol, 1 equiv) in DMF (30 mL), was added 3-aminobenzoic acid (1000 mg, 0.0073 mol, 1 equiv), N,N-Diisopropylethylamine (2830 mg, 0.0219 mol, 3 equiv), and 1-[Bis(dimethylamino)methylene]-H-1-1,2,3-triazolo[4,5-b]pyridinium 3-oxid hexafluorophosphate (3050 mg, 0.0080 mol, 1.1 equiv). The resultant reaction mixture was heated at 80° C. for 2 h. Reaction was monitored by LCMS. After completion of reaction, water (50 ml) was added to the reaction mixture and extracted with ethyl acetate (100 mL×2). Combined all organic layers and washed with water (50 mL×8) and brine solution (100 mL). The organic layer was dried over anhydrous sodium sulphate and concentrated under reduced pressure to obtain desired product. LCMS: 220 [M+H]⁺

Step—2: Synthesis of 8-cyclopentyl-2-((3-(4-methylpiperazine-1-carbonyl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (60 mg, 0.198 mmol, 1 equiv) in toluene (3 mL), was added (3-aminophenyl)(4-methylpiperazin-1-yl)methanone (48.07 mg, 0.219 mmol, 1.1 equiv). The resultant reaction mixture was heated at 100° C. for 2 h. Reaction was monitored by LCMS. After completion of reaction, solid precipitated out was filtered and purified by reverse phase HPLC to afford desired product: LCMS: 458 [M+H]⁺; ¹H NMR: (400 MHz, Chloroform-d) δ 8.62 (s, 1H), 8.02 (s, 1H), 7.84-7.73 (m, 1H), 7.65 (s, 1H), 7.42 (t, J=8.0 Hz, 1H), 7.18-7.09 (m, 1H), 5.85 (t, J=9.0 Hz, 1H), 2.74 (s, 6H), 2.4 (s, 3H), 2.25 (s, 2H), 1.99 (dd, J=15.3, 6.8 Hz, 3H), 1.84 (d, J=9.2 Hz, 2H), 1.64 (d, J=9.4 Hz, 2H).

### Example—S57: Synthesis of 8-cyclopentyl-2-((3-((4-methylpiperazin-1-yl)sulfonyl)phenyl)amino)-7-oxo-7,8-dihydropyrido[2,3-d]pyrimidine-6-carbonitrile (Compound No. 17)

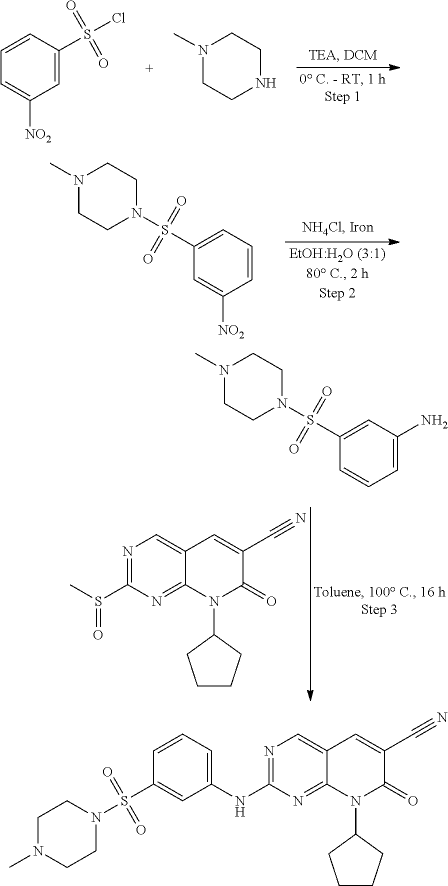

Step—1: Synthesis of 1-methyl-4-((3-nitrophenyl)sulfonyl)piperazine: To a stirred solution of 1-methylpiperazine (1.2 g, 0.0108 mol, 1.2 equiv) in DCM (20 mL), was dropwise added triethylamine (1.82 g, 0.018 mol, 2 equiv) at 0° C. The resultant reaction mixture was stirred at 0° C. for 10 min. Then 3-nitrobenzenesulfonyl chloride (2 gm, 0.009 mol, 1 equiv) was added and stirred for 1 h at RT. Reaction was monitored by TLC. After completion of reaction, water was added to the reaction mixture and extracted with DCM (20 mL×2). Combined all organic layers and washed with water (20 mL×2) and brine solution (20 mL). The organic layer was dried over anhydrous sodium sulphate and concentrated under reduced pressure to obtain desired product. LCMS: 286 [M+H]⁺

Step—2: Synthesis of 3-((4-methylpiperazin-1-yl)sulfonyl)aniline: To a stirred solution of 1-methyl-4-((3-nitrophenyl)sulfonyl)piperazine (2.6 g, 0.009 mol, 1 equiv) in EtOH (30 mL), was added Iron (5.03 g, 0.09 mol, 10 equiv), ammonium chloride (4.81 g, 0.09 mol, 10 equiv) and water (10 ml). The resultant reaction mixture was heated at 80° C. for 2 h. Reaction mixture was filtered through the celite. The filtrate was concentrated under reduced pressure. Aqueous layer was extracted by the ethyl acetate (30 mL×3). All organic layers was combined and washed by brine solution (20 mL). The organic layer was dried over anhydrous sodium sulphate and concentrated under reduced pressure to obtain desired product. LCMS: 256 [M+H]⁺

Step—3: Synthesis of 8-cyclopentyl-2-((3-((4-methylpiperazin-1-yl)sulfonyl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (50 mg, 0.175 mmol, 1 equiv) in toluene (2 mL), was added 4-((4-methylpiperazin-1-yl)sulfonyl)aniline (49.09 mg, 0.192 mmol, 1.1 equiv). The resultant reaction mixture was heated at 100° C. for 2 h. Reaction was monitored by LCMS. After completion of reaction, solid precipitated out was filtered and purified by reverse phase HPLC to afford desired product. LCMS: 494 [M+H]⁺; ¹H NMR: (400 MHz, Methanol-d4) δ 8.83 (s, 1H), 8.59 (s, 1H), 8.41 (s, 1H), 7.78 (s, 1H), 7.62 (t, J=7.9 Hz, 1H), 7.52 (d, J=7.8 Hz, 1H), 6.09 (p, J=8.2 Hz, 1H), 3.19 (s, 4H), 2.93 (s, 5H), 2.56 (s, 4H), 2.24 (t, J=9.0 Hz, 3H), 2.11-1.91 (m, 2H), 1.76 (t, J=6.6 Hz, 2H).

### Example—S58: Synthesis of 8-cyclopentyl-2-((3-((4-hydroxypiperidin-1-yl)sulfonyl)phenyl)amino)-7-oxo-7,8-dihydropyrido[2,3-d]pyrimidine-6-carbonitrile (Compound No. 18)

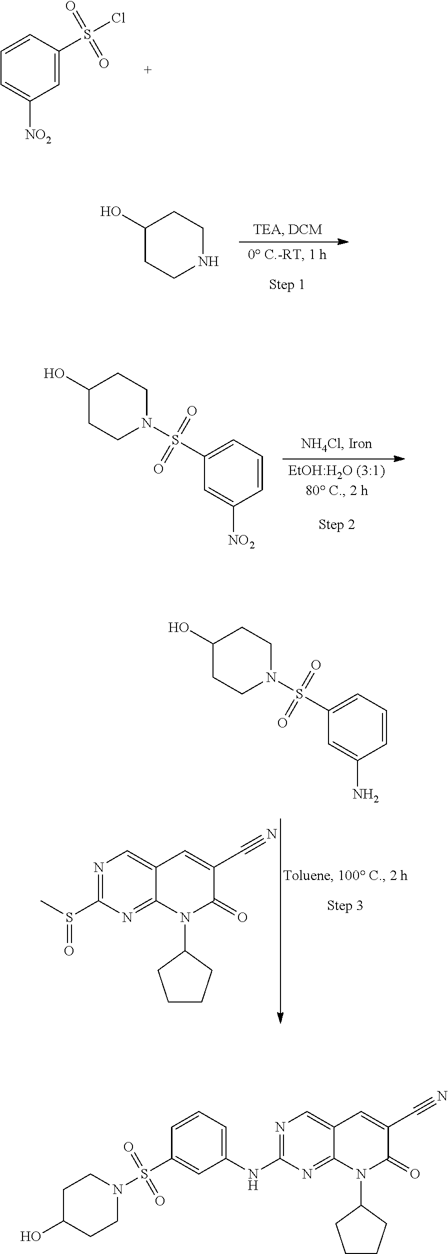 [SMILES: O=[N+]([O-])c1cccc(S(=O)(=O)Cl)c1 ; OC1CCNCC1 ; O=[N+]([O-])c1cccc(S(=O)(=O)N2CCC(O)CC2)c1 ; Nc1cccc(S(=O)(=O)N2CCC(O)CC2)c1 ; CS(=O)c1ncc2cc(C#N)c(=O)n(C3CCCC3)c2n1 ; N#Cc1cc2cnc(Nc3cccc(S(=O)(=O)N4CCC(O)CC4)c3)nc2n(C2CCCC2)c1=O]

Step—1: Synthesis of 1-((3-nitrophenyl)sulfonyl)piperidin-4-ol: To a stirred solution of piperidin-4-ol (1.1 g, 0.0108 mmol, 1.2 equiv) in DCM (20 mL), was dropwise added triethylamine (1.82 g, 0.018 mmol, 2 equiv) at 0° C. The resultant reaction mixture was stirred at 0° C. for 10 min. Then 3-nitrobenzenesulfonyl chloride (2 gm, 0.009 mmol, 1 equiv) was added and stirred for 1 h at RT. Reaction was monitored by TLC. After completion of reaction, water was added to the reaction mixture and extracted with DCM (20 mL×2). Combined organic layers and washed with water (20 mL×3) and brine solution (20 mL). The organic layer was dried over anhydrous sodium sulphate and concentrated under reduced pressure to obtain desired product. LCMS: 287 [M+H]⁺

Step—2: Synthesis of 1-((3-aminophenyl)sulfonyl)piperidin-4-ol: To a stirred solution of 1-((3-nitrophenyl)sulfonyl)piperidin-4-ol (2.2 g, 0.0077 mol, 1 equiv) in EtOH (30 mL), was added Iron (4.30 g, 0.077 mmol, 10 equiv), ammonium chloride (4.11 g, 0.077 mmol, 10 equiv) and water (10 mL). The resultant reaction mixture was heated at 80° C. for 2 h. Reaction mixture was filtered through the celite. The filtrate was concentrated under reduced pressure. Aqueous layer was extracted by the ethyl acetate (30 mL×3). All organic layers was combined and washed by brine solution (20 mL). The organic layer was dried over anhydrous sodium sulphate and concentrated under reduced pressure to obtain desired product. LCMS: 257 [M+H]⁺

Step—3: Synthesis of 8-cyclopentyl-2-((3-((4-hydroxypiperidin-1-yl)sulfonyl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (50 mg, 0.165 mmol, 1 equiv) in toluene (2 mL), was added 1-((3-aminophenyl)sulfonyl)piperidin-4-ol (46.08 mg, 0.18 mmol, 1.1 equiv). The resultant reaction mixture was heated at 100° C. for 2 h. Reaction was monitored by LCMS. After completion of reaction, solid precipitated out was filtered and purified by reverse phase HPLC to afford desired product. LCMS: 495 [M+H]⁺; ¹H NMR: (400 MHz, DMSO-d₆) δ 10.87 (s, 1H), 8.91 (s, 1H), 8.62 (s, 1H), 8.50 (s, 1H), 7.84 (s, 1H), 7.63 (t, J=7.9 Hz, 1H), 7.43 (d, J=7.5 Hz, 1H), 5.94 (p, J=8.4 Hz, 1H), 4.68 (d, J=3.8 Hz, 1H), 3.54 (dt, J=8.3, 4.4 Hz, 4H), 3.19-3.10 (m, 4H), 2.76 (ddd, J=11.6, 8.0, 3.4 Hz, 2H), 2.12 (q, J=8.8, 6.5 Hz, 2H), 1.86 (d, J=10.0 Hz, 2H), 1.80-1.61 (m, 4H).

### Example—S59: Synthesis of 8-cyclopentyl-2-(3-(morpholinosulfonyl)phenyl)-7-oxo-7,8-dihydropyrido[2,3-d]pyrimidine-6-carbonitrile (Compound No. 20)

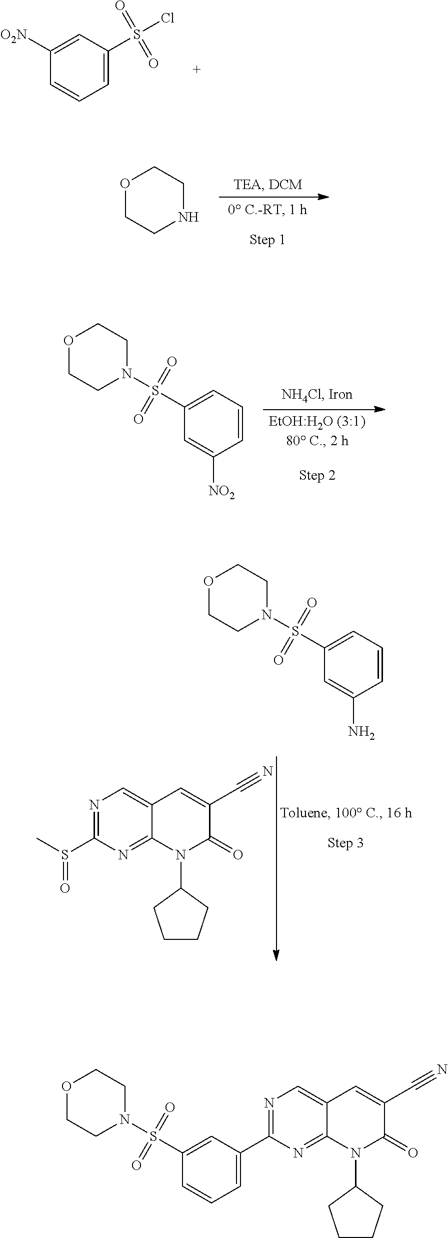 [SMILES: O=[N+]([O-])c1cccc(S(=O)(=O)Cl)c1 ; C1COCCN1 ; O=[N+]([O-])c1cccc(S(=O)(=O)N2CCOCC2)c1 ; Nc1cccc(S(=O)(=O)N2CCOCC2)c1 ; CS(=O)c1ncc2cc(C#N)c(=O)n(C3CCCC3)c2n1 ; N#Cc1cc2cnc(-c3cccc(S(=O)(=O)N4CCOCC4)c3)nc2n(C2CCCC2)c1=O]

Step—1: Synthesis of 4-((3-nitrophenyl)sulfonyl)morpholine: To a stirred solution of morpholine (0.708 g, 8.14 mmol, 1.2 equiv) in DCM (15 mL), was dropwise added triethylamine (1.372 g, 1.372 mmol, 2 equiv) at 0° C. The resultant reaction mixture was stirred at 0° C. for 10 min. Then 3-nitrobenzenesulfonyl chloride (1.5 g, 6.79 mmol, 1 equiv) was added and stirred for 1 h at RT. Reaction was monitored by TLC. After completion of reaction, water was added to the reaction mixture and extracted with DCM (20 mL×2). Combined all organic layers and washed with water (20 mL×2) and brine solution (20 mL). The organic layer was dried over anhydrous sodium sulphate and concentrated under reduced pressure to obtain desired product. LCMS: 273 [M+H]⁺

Step—2: Synthesis of 3-(morpholinosulfonyl)aniline: To a stirred solution of 4-((3-nitrophenyl)sulfonyl)morpholine (2.2 g, 0.008 mol, 1 equiv) in EtOH (30 mL), was added Iron (4.51 g, 0.08 mol, 10 equiv), ammonium chloride (4.32 g, 0.08 mol, 10 equiv) and water (10 ml). The resultant reaction mixture was heated at 80° C. for 2 h. Reaction mixture was filtered through the celite. The filtrate was concentrated under reduced pressure. Aqueous layer was extracted by the ethyl acetate (30 mL×3). All organic layers was combined and washed by brine solution (20 mL). The organic layer was dried over anhydrous sodium sulphate and concentrated under reduced pressure to obtain desired product. LCMS: 243 [M+H]⁺

Step—3: Synthesis of 8-cyclopentyl-2-(3-(morpholinosulfonyl)phenyl)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (75 mg, 0.248 mmol, 1 equiv) in toluene (2 mL), was added 3-(morpholinosulfonyl)aniline (66 mg, 0.273 mmol, 1.1 equiv). The resultant reaction mixture was heated at 100° C. for 2 h. Reaction was monitored by LCMS. After completion of reaction, solid precipitated out was filtered and purified by reverse phase HPLC to afford desired product. LCMS: 481 [M+H]⁺; ¹H NMR: (400 MHz, Methanol-d4) δ 8.83 (s, 1H), 8.55 (s, 2H), 8.42 (s, 1H), 7.78 (s, 1H), 7.62 (t, J=8.0 Hz, 1H), 7.50 (d, J=7.6 Hz, 1H), 6.10 (t, J=8.6 Hz, 1H), 3.75-3.63 (m, 4H), 3.00 (t, J=4.7 Hz, 4H), 2.28-2.13 (m, 2H), 2.10-1.93 (m, 3H), 1.75 (s, 2H).

### Example—S60: Synthesis of 8-cyclopentyl-2-((5-(3-methylpiperazin-1-yl)pyridin-2-yl)amino)-7-oxo-7,8-dihydropyrido[2,3-d]pyrimidine-6-carbonitrile (Compound No. 23)

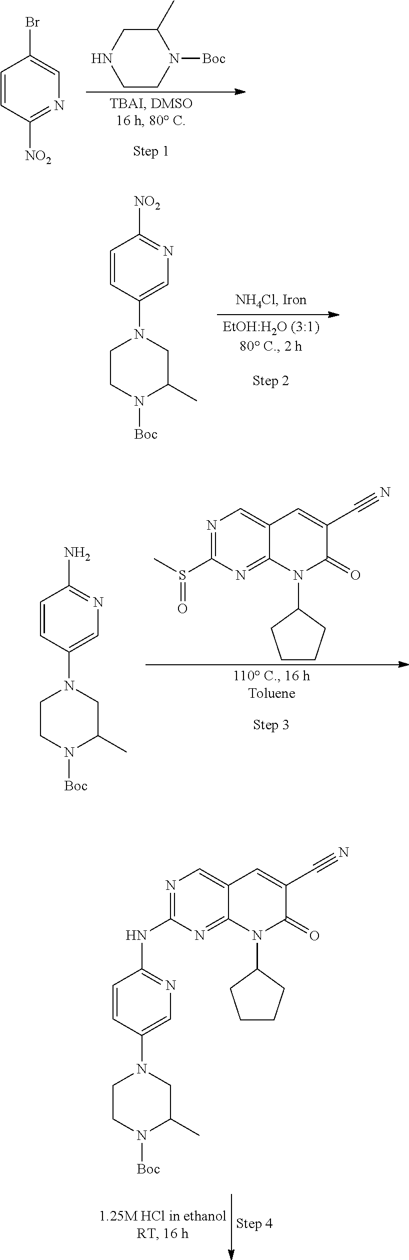 [SMILES: O=[N+]([O-])c1ccc(Br)cn1 ; CC1CNCCN1C(=O)OC(C)(C)C ; CC1CN(c2ccc([N+](=O)[O-])nc2)CCN1C(=O)OC(C)(C)C ; CC1CN(c2ccc(N)nc2)CCN1C(=O)OC(C)(C)C ; CS(=O)c1ncc2cc(C#N)c(=O)n(C3CCCC3)c2n1 ; CC1CN(c2ccc(Nc3ncc4cc(C#N)c(=O)n(C5CCCC5)c4n3)nc2)CCN1C(=O)OC(C)(C)C]

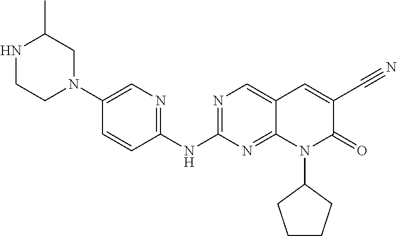 [SMILES: CC1CN(c2ccc(Nc3ncc4cc(C#N)c(=O)n(C5CCCC5)c4n3)nc2)CCN1]

Step—1: Synthesis of tert-butyl 2-methyl-4-(6-nitropyridin-3-yl)piperazine-1-carboxylate: To a solution of 5-bromo-2-nitropyridine (2 g, 9.85 mmol, 1 equiv) in DMSO (12 mL) was added Tetrabutyl ammonium iodide (1.83 g, 9.85 mmol, 1 equiv) followed by tert-butyl 2-methylpiperazine-1-carboxylate (2.38 g, 11.82 mmol, 1.2 equiv). Resultant reaction mixture was stirred at 80° C. for 16 h. Reaction was monitored by TLC and LCMS. After completion of reaction, reaction mass was diluted with ice water (50 mL), and extracted with ethyl acetate (100 ml). Organic layer was dried over anhydrous sodium sulphate, filtered and concentrated under reduced pressure to obtained crude product. Compound was purified by column chromatography (27% ethyl acetate in hexane) to obtain desired product. LCMS: 323 [M+H]⁺

Step—2: Synthesis of tert-butyl 4-(6-aminopyridin-3-yl)-2-methylpiperazine-1-carboxylate: To a stirred solution of tert-butyl 2-methyl-4-(6-nitropyridin-3-yl)piperazine-1-carboxylate (2.5 g, 7.76 mmol, 1 equiv) in EtOH (60 mL), was added Iron (4.33 g, 77.6 mmol, 10 equiv), ammonium chloride (4.306 g, 77.6 mmol, 10 equiv) and water (20 ml). The resultant reaction mixture was heated at 80° C. for 2 h. Reaction mixture was filtered through the celite. The filtrate was concentrated under reduced pressure. Aqueous layer was extracted by the ethyl acetate (60 mL×3). Combined organic layer was washed by brine solution (30 mL). The organic layer was dried over anhydrous sodium sulphate and concentrated under reduced pressure to obtain desired product. LCMS: 293 [M+H]⁺

Step—3: Synthesis of tert-butyl 4-(6-((6-cyano-8-cyclopentyl-7-oxo-7,8-dihydropyrido[2,3-d]pyrimidin-2-yl)amino)pyridin-3-yl)-2-methylpiperazine-1-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (4 mL), was added obtain tert-butyl 4-(6-aminopyridin-3-yl)-2-methylpiperazine-1-carboxylate (106 mg, 0.363 mmol, 1.1 equiv). The resultant reaction mixture was heated at 110° C. for 2 h. Reaction was monitored by LCMS. After completion of reaction, solid precipitate out was concentrated and purified the recrystallization in methanol to afford desired product. LCMS: 531 [M+H]⁺

Step—4: Synthesis of 8-cyclopentyl-2-((5-(3-methylpiperazin-1-yl)pyridin-2-yl)amino)-7-oxo-7,8-dihydropyrido[2,3-d]pyrimidine-6-carbonitrile: To a solution of tert-butyl 4-(6-((6-cyano-8-cyclopentyl-7-oxo-7,8-dihydropyrido[2,3-d]pyrimidin-2-yl)amino)pyridin-3-yl)-2-methylpiperazine-1-carboxylate (50 mg) in 1.25 M HCl in ethanol (5 mL). The resultant reaction mixture was stirred at RT for 16 h. Reaction was monitored by LCMS. After completion of reaction, filtered the solid product and washed with diethyl ether and dried under reduced pressure to afford desired product. LCMS: 431 [M+H]⁺, ¹H NMR: (400 MHz, Methanol-d4) δ 9.03 (s, 1H), 8.57 (s, 1H), 8.16 (d, J=9.5 Hz, 1H), 8.04 (d, J=2.9 Hz, 1H), 7.69 (d, J=9.5 Hz, 1H), 6.01 (p, J=8.9 Hz, 1H), 3.98-3.84 (m, 2H), 3.63-3.54 (m, 2H), 3.38 (dd, J=12.3, 3.8 Hz, 1H), 3.35 (s, 3H), 3.19 (td, J=12.4, 11.9, 2.9 Hz, 1H), 2.96 (dd, J=13.2, 10.6 Hz, 1H), 2.30 (dq, J=14.0, 7.4 Hz, 2H), 2.14-1.99 (m, 2H), 1.92 (dt, J=11.5, 7.9 Hz, 2H), 1.71 (p, J=6.2, 5.0 Hz, 2H), 1.44 (d, J=6.6 Hz, 3H).

### Example—S61: Synthesis of 8-cyclopentyl-2-((4-(4-methylpiperidin-1-yl)phenyl)amino)-7-oxo-7,8-dihydropyrido[2,3-d]pyrimidine-6-carbonitrile (Compound No. 25)

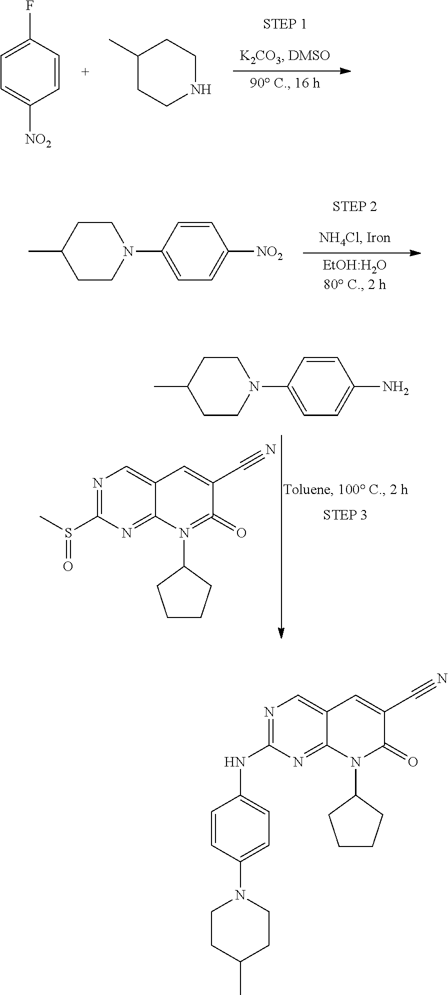

Step—1: Synthesis of 4-methyl-1-(4-nitrophenyl)piperidine: To a stirred solution of 1-fluoro-4-nitrobenzene (1.2 g, 8.5 mmol, 1 equiv) in DMSO (15 mL), was added potassium carbonate (2 g, 14.45 mmol, 1.7 equiv) and 4-methylpiperidine (1.27 g, 12.7 mmol, 1.5 equiv). The resultant reaction mixture was stirred at 90° C. for 16 h. Reaction was monitored by TLC and LCMS. After completion of reaction, ice-water (100 mL) was added to the reaction mixture, product was precipitated and filtered the precipitated product, and dried under reduced pressure to obtain desired product. LCMS: 221 [M+H]⁺

Step—2: Synthesis of 4-(4-methylpiperidin-1-yl)aniline: To a stirred suspension of 4-methyl-1-(4-nitrophenyl)piperidine (1.8 g, 8.18 mmol, 1 equiv) in EtOH (20 mL), was added Iron (4.4 g, 81.8 mmol, 10 equiv), ammonium chloride (4.6 g, 81.8 mol, 10 equiv) and water (5 ml). The resultant reaction mixture was heated at 80° C. for 2 h. Reaction mixture was filtered through the celite. The filtrate was concentrated under reduced pressure. Aqueous layer was extracted by the ethyl acetate (30 mL×3). All organic layers were combined and washed by brine solution (20 mL). Combined organic layer was dried over anhydrous sodium sulphate and concentrated under reduced pressure to obtain desired product. LCMS: 191 [M+H]⁺

Step—3: Synthesis of 8-cyclopentyl-2-((4-(4-methylpiperidin-1-yl)phenyl)amino)-7-oxo-7,8-dihydropyrido[2,3-d]pyrimidine-6-carbonitrile: To a suspension of 8-cyclopentyl-2-(methylsulfinyl)-7-oxo-7,8-dihydropyrido[2,3-d]pyrimidine-6-carbonitrile (50 mg, 0.16 mmol, 1 equiv) in toluene (3 mL), was added 4-(4-methylpiperidin-1-yl)aniline (35 mg, 0.18 mmol, 1.1 equiv). Resultant reaction mixture was stirred at 100° C. for 2 h. Reaction was monitored by LCMS. After completion of reaction, reaction mixture was concentrated under reduced pressure. Product was triturated from methanol to afford desired product. LCMS: 429 [M+H]⁺, ¹H NMR: (400 MHz, DMSO-d₆) δ 10.33 (s, 1H), 8.77 (s, 1H), 8.51 (s, 1H), 7.46 (d, J=8.5 Hz, 2H), 7.25 (t, J=7.3 Hz, 0H), 7.20-7.12 (m, OH), 6.93 (d, J=8.6 Hz, 2H), 5.72 (s, 1H), 3.67-3.59 (m, 2H), 2.62 (t, J=12.3 Hz, 2H), 2.30 (s, OH), 2.18 (dt, J=15.8, 7.2 Hz, 2H), 2.01 (t, J=9.4 Hz, 0H), 1.83-1.64 (m, 6H), 1.55 (s, 3H), 1.53-1.43 (m, 1H), 1.30-1.16 (m, 2H), 0.93 (d, J=6.5 Hz, 3H).

### Example—S62: Synthesis of 8-cyclopentyl-2-((2-fluoro-4-(piperazin-1-yl)phenyl)amino)-7-oxo-7,8-dihydropyrido[2,3-d]pyrimidine-6-carbonitrile (Compound No. 26)

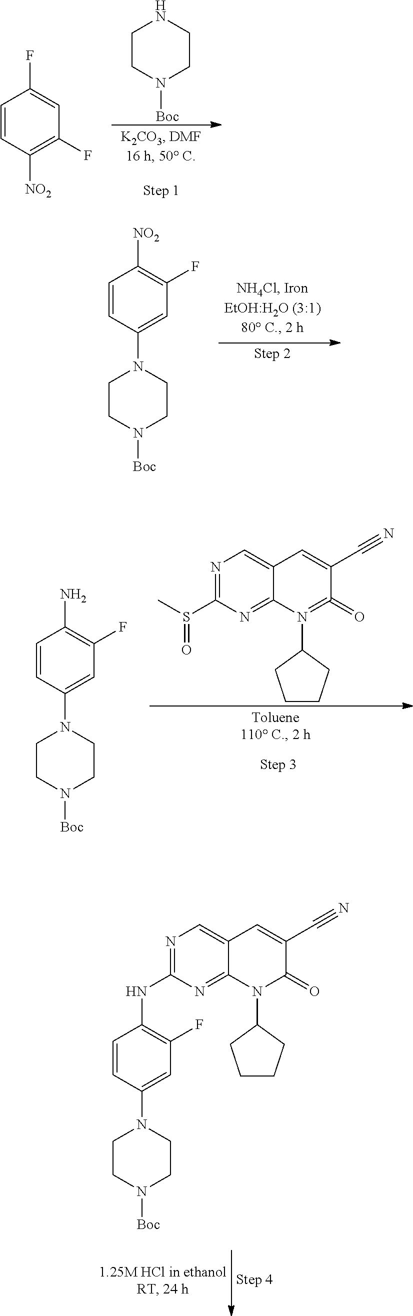

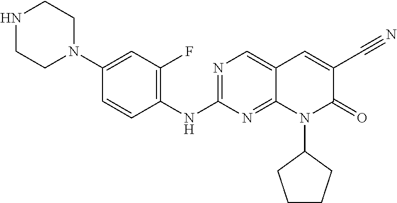 [SMILES: N#Cc1cc2cnc(Nc3ccc(N4CCNCC4)cc3F)nc2n(C2CCCC2)c1=O]

Step—1: Synthesis of tert-butyl 4-(3-fluoro-4-nitrophenyl)piperazine-1-carboxylate: To a solution of 2,4-difluoro-1-nitrobenzene (1 g, 5.38 mmol, 1 equiv) in DMF (6 mL) was added tert-butyl piperazine-1-carboxylate (0.941 g, 5.92 mmol, 1.1 equiv) followed by potassium carbonate (1.48 g, 10.76 mmol, 2 equiv). Resultant reaction mixture was stirred at 50° C. for 16 h. Reaction was monitored by TLC and LCMS. After completion of reaction, reaction mass was diluted with water (15 mL) and extracted with ethyl acetate (3×20 mL). Combined all organic layers and washed with water (10×15 mL). The organic layer was dried by anhydrous sodium sulphate, filtered and concentrated under reduced pressure to obtained crude product. Compound was purified by column chromatography (0-30% ethyl acetate in hexane) to obtain desired product. LCMS: 326 [M+H]⁺

Step—2: Synthesis of tert-butyl 4-(4-amino-3-fluorophenyl)piperazine-1-carboxylate: To a stirred solution tert-butyl 4-(3-fluoro-4-nitrophenyl)piperazine-1-carboxylate (600 mg, 1.85 mmol, 1 equiv) in EtOH (18 mL), was added Iron (1.033 g, 18.5 mmol, 10 equiv), ammonium chloride (1.026 g, 18.5 mmol, 10 equiv) and water (6 ml). The resultant reaction mixture was heated at 80° C. for 2 h. Reaction mixture was filtered through the celite. The filtrate was concentrated under reduced pressure. Aqueous layer was extracted by the ethyl acetate (3×20 mL). Combined organic layer was washed by brine solution (20 mL). The organic layer was dried over anhydrous sodium sulphate and concentrated under reduced pressure to afford desired product. LCMS: 296 [M+H]⁺

Step—3: Synthesis of tert-butyl 4-(4-((6-cyano-8-cyclopentyl-7-oxo-7,8-dihydropyrido[2,3-d]pyrimidin-2-yl)amino)-3-fluorophenyl)piperazine-1-carboxylate: To a stirred solution of 8-cyclopentyl-2-(methylsulfinyl)-7-oxo-7,8-dihydropyrido[2,3-d]pyrimidine-6-carbonitrile (75 mg, 0.248 mmol, 1 equiv) in toluene (4 mL), was added obtain tert-butyl 4-(4-amino-3-fluorophenyl)piperazine-1-carboxylate (80.5 mg, 0.273 mmol, 1.1 equiv). The resultant reaction mixture was heated at 110° C. for 2 h. Reaction was monitored by LCMS. After completion of reaction, solid precipitate was concentrated and purified the recrystallization in methanol to afford desired product. LCMS: 534 [M+H]⁺

Step—4: Synthesis of 8-cyclopentyl-2-((2-fluoro-4-(piperazin-1-yl)phenyl)amino)-7-oxo-7,8-dihydropyrido[2,3-d]pyrimidine-6-carbonitrile: To a solution of tert-butyl 4-(4-((6-cyano-8-cyclopentyl-7-oxo-7,8-dihydropyrido[2,3-d]pyrimidin-2-yl)amino)-3-fluorophenyl)piperazine-1-carboxylate (46 mg) in 1.25M HCl in ethanol (5 mL). The resultant reaction mixture was stirred at RT for 24 h. Reaction was monitored by LCMS. After completion of reaction, filtered the solid product and concentrated under reduced pressure to afford desired product. LCMS: 434 [M+H]⁺, ¹H NMR: (400 MHz, Methanol-d4) δ 8.78-8.71 (m, 1H), 8.37 (s, 1H), 6.99-6.87 (m, 2H), 3.48 (s, 4H), 3.38 (s, 4H), 2.26-2.16 (m, 2H), 1.77-1.69 (m, 2H), 1.55-1.45 (m, 2H).

### Example—S63: Synthesis of 8-cyclopentyl-2-((3,5-difluoro-4-(piperazin-1-yl)phenyl)amino)-7-oxo-7,8-dihydropyrido[2,3-d]pyrimidine-6-carbonitrile (Compound No. 27)

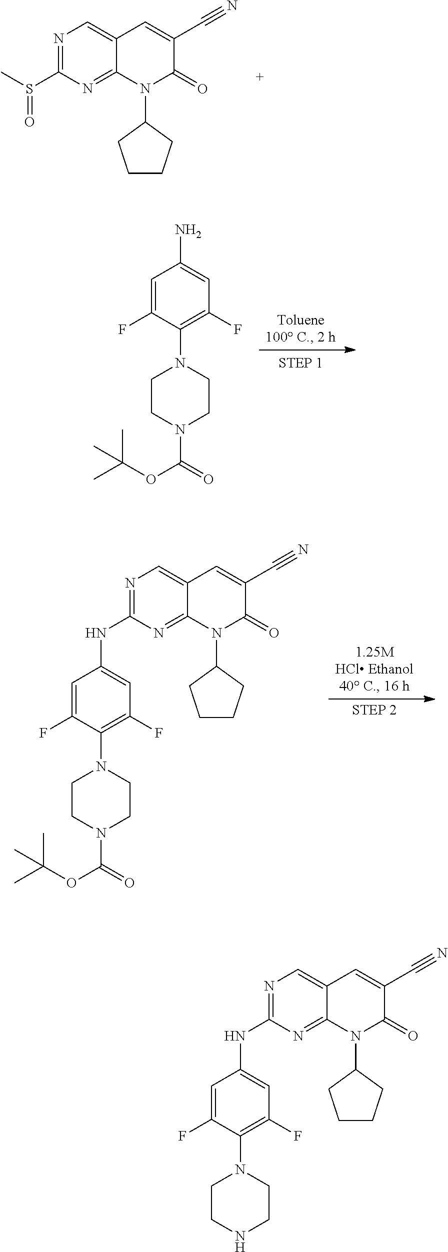 [SMILES: CS(=O)c1ncc2cc(C#N)c(=O)n(C3CCCC3)c2n1 ; CC(C)(C)OC(=O)N1CCN(c2c(F)cc(N)cc2F)CC1 ; CC(C)(C)OC(=O)N1CCN(c2c(F)cc(Nc3ncc4cc(C#N)c(=O)n(C5CCCC5)c4n3)cc2F)CC1 ; N#Cc1cc2cnc(Nc3cc(F)c(N4CCNCC4)c(F)c3)nc2n(C2CCCC2)c1=O]

Step—1: Synthesis of tert-butyl 4-(4-((6-cyano-8-cyclopentyl-7-oxo-7,8-dihydropyrido[2,3-d]pyrimidin-2-yl)amino)-2,6-difluorophenyl)piperazine-1-carboxylate: To a suspension of 8-cyclopentyl-2-(methylsulfinyl)-7-oxo-7,8-dihydropyrido[2,3-d]pyrimidine-6-carbonitrile (50 mg, 0.16 mmol, 1 equiv) in toluene (3 mL), was added tert-butyl 4-(4-amino-2,6-difluorophenyl)piperazine-1-carboxylate (57 mg, 0.18 mmol, 1.1 equiv). Resultant reaction mixture was stirred at 100° C. for 2 h. Reaction was monitored by LCMS. After completion of reaction, reaction mixture was concentrated under reduced pressure. Product was triturated from methanol to afford the desired product: LCMS: 552 [M+H]⁺

Step—2: Synthesis of 8-cyclopentyl-2-((3,5-difluoro-4-(piperazin-1-yl)phenyl)amino)-7-oxo-7,8-dihydropyrido[2,3-d]pyrimidine-6-carbonitrile: To a solution of tert-butyl 4-(4-((6-cyano-8-cyclopentyl-7-oxo-7,8-dihydropyrido[2,3-d]pyrimidin-2-yl)amino)-2,6-difluorophenyl)piperazine-1-carboxylate (20 mg) was added to the 1.25M HCl in ethanol (3 mL). The resultant reaction mixture was stirred at 40° C. for 16 h. Reaction was monitored by LCMS. After completion of reaction, filtered the solid product and washed with diethyl ether and dried under reduced pressure to afford desired product. LCMS: 452 [M+H]⁺, ¹H NMR: (400 MHz, Methanol-d4) δ 8.82 (s, 1H), 8.41 (s, 1H), 7.46 (d, J=11.5 Hz, 2H), 5.98 (q, J=9.0 Hz, 1H), 3.45-3.38 (m, 4H), 3.36 (d, J=6.1 Hz, 5H), 2.32 (p, J=8.0, 7.4 Hz, 2H), 2.11-1.99 (m, 3H), 1.90 (dd, J=13.2, 8.0 Hz, 2H), 1.70 (q, J=5.8, 5.0 Hz, 2H), 1.32-1.22 (m, 2H), 0.89 (q, J=8.0, 7.2 Hz, 1H).

### Example—S64: Synthesis of 2-((3-chloro-4-(4-methylpiperazin-1-yl)phenyl)amino)-8-cyclopentyl-7-oxo-7,8-dihydropyrido[2,3-d]pyrimidine-6-carbonitrile (Compound No. 28)

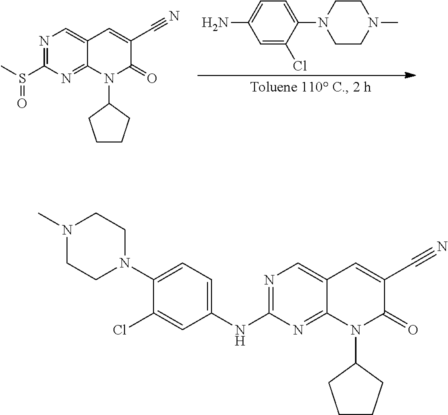 [SMILES: CS(=O)c1ncc2cc(C#N)c(=O)n(C3CCCC3)c2n1 ; CN1CCN(c2ccc(N)cc2Cl)CC1 ; CN1CCN(c2ccc(Nc3ncc4cc(C#N)c(=O)n(C5CCCC5)c4n3)cc2Cl)CC1]

To a suspension of 8-cyclopentyl-2-(methylsulfinyl)-7-oxo-7,8-dihydropyrido[2,3-d]pyrimidine-6-carbonitrile (50 mg, 0.16 mmol, 1 equiv) in toluene (3 mL), was added 3-chloro-4-(4-methylpiperazin-1-yl)aniline (41 mg, 0.18 mmol, 1.1 equiv). Resultant reaction mixture was stirred at 110° C. for 2 h. Reaction was monitored by LCMS. After completion of reaction, reaction mixture was concentrated under reduced pressure. Product was triturated from methanol to afford desired product. LCMS: 464.3 [M+H]⁺; ¹H NMR: (400 MHz, Chloroform-d) δ 8.59 (s, 1H), 7.98 (s, 1H), 7.92 (s, 1H), 7.41 (s, 1H), 7.19 (s, 1H), 7.07 (d, J=8.6 Hz, 1H), 5.86 (p, J=8.8 Hz, 1H), 3.49 (s, 1H), 3.10 (s, 4H), 2.64 (s, 4H), 2.38 (s, 3H), 2.28 (dq, J=15.5, 7.7 Hz, 3H), 2.01 (s, 3H), 1.89 (dq, J=11.6, 6.9, 4.1 Hz, 2H), 1.68 (dt, J=11.1, 7.1 Hz, 2H), 1.25 (s, 1H), 0.84 (s, 1H).

### Example—S65: Synthesis of 2-((3-chloro-4-(piperazin-1-yl)phenyl)amino)-8-cyclopentyl-7-oxo-7,8-dihydropyrido[2,3-d]pyrimidine-6-carbonitrile (Compound No. 30)

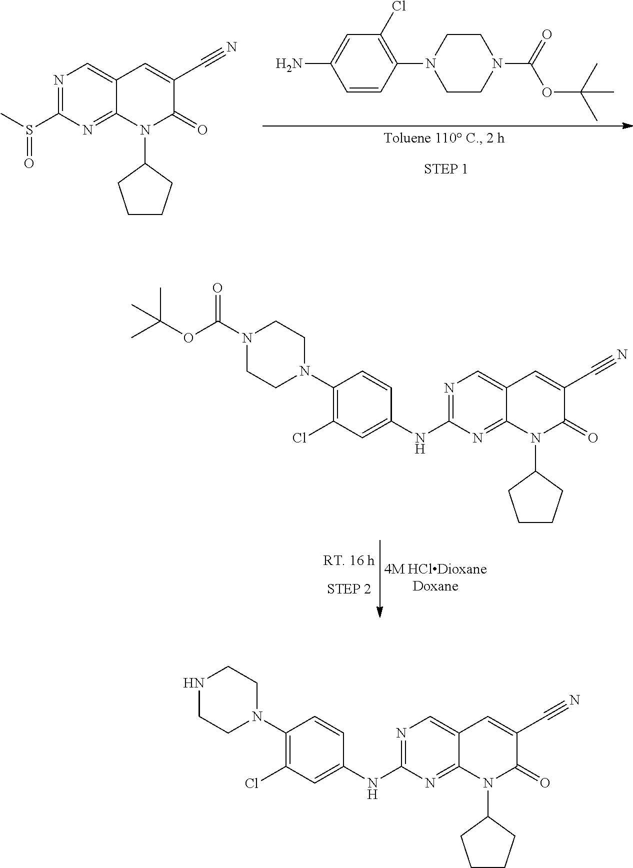 [SMILES: CS(=O)c1ncc2cc(C#N)c(=O)n(C3CCCC3)c2n1 ; CC(C)(C)OC(=O)N1CCN(c2ccc(N)cc2Cl)CC1 ; CC(C)(C)OC(=O)N1CCN(c2ccc(Nc3ncc4cc(C#N)c(=O)n(C5CCCC5)c4n3)cc2Cl)CC1 ; N#Cc1cc2cnc(Nc3ccc(N4CCNCC4)c(Cl)c3)nc2n(C2CCCC2)c1=O]

Step—1: Synthesis of tert-butyl 4-(2-chloro-4-((6-cyano-8-cyclopentyl-7-oxo-7,8-dihydropyrido[2,3-d]pyrimidin-2-yl)amino)phenyl)piperazine-1-carboxylate: To a suspension of 8-cyclopentyl-2-(methylsulfinyl)-7-oxo-7,8-dihydropyrido[2,3-d]pyrimidine-6-carbonitrile (75 mg, 0.248 mmol, 1 equiv) in toluene (3 mL), was added tert-butyl 4-(4-amino-2-chlorophenyl)piperazine-1-carboxylate (85 mg, 0.273 mmol, 1.1 equiv). Resultant reaction mixture was stirred at 110° C. for 2 h. Reaction was monitored by LCMS. After completion of reaction, reaction mixture was concentrated under reduced pressure. Product was triturated from methanol to afford desired product. LCMS: 550.4 [M+H]⁺

Step—2: Synthesis of 2-((3-chloro-4-(piperazin-1-yl)phenyl)amino)-8-cyclopentyl-7-oxo-7,8-dihydropyrido[2,3-d]pyrimidine-6-carbonitrile: To a solution of tert-butyl 4-[2-chloro-4-[(6-cyano-8-cyclopentyl-7-oxo-pyrido[2,3-d]pyrimidin-2-yl)amino]phenyl]piperazine-1-carboxylate (75 mg, 0.14 mmol, 1 equiv) in dioxane (1 mL) was added to the 4M HCl in dioxane (2 mL). The resultant reaction mixture was stirred at RT for 16 h. Reaction was monitored by LCMS. After completion of reaction, reaction mixture was concentrated under reduced pressure to afford desired product. LCMS: 450.4 [M+H]⁺, ¹H NMR: (400 MHz, DMSO-d₆) δ 10.65 (s, 1H), 9.14 (s, 5H), 8.86 (s, 1H), 8.59 (s, 1H), 8.05 (s, 1H), 7.49 (s, 1H), 7.23 (d, J=8.6 Hz, 1H), 5.80 (s, 1H), 3.23 (s, 3H), 3.20-3.13 (m, 3H), 2.19 (q, J=8.7, 7.8 Hz, 2H), 1.95-1.86 (m, 1H), 1.81 (q, J=10.1, 9.6 Hz, 2H), 1.65-1.54 (m, 2H), 1.34 (s, 4H).

### Example—S66: Synthesis of 8-benzyl-2-((4-(4-methylpiperazin-1-yl)phenyl)amino)-7-oxo-7,8-dihydropyrido[2,3-d]pyrimidine-6-carbonitrile (Compound No. 32)

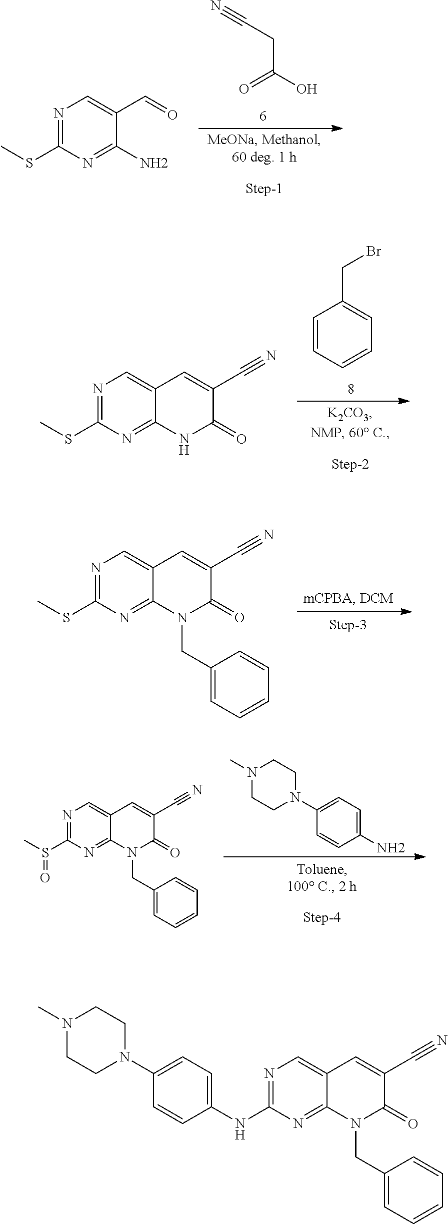

Step—1: Synthesis of 2-(methylthio)-7-oxo-7,8-dihydropyrido[2,3-d]pyrimidine-6-carbonitrile: To a stirred solution of NaOMe (324 mg, 5.9 mmol, 2.0 equiv.) in methanol (15 mL), was added 4-amino-2-(methylthio)pyrimidine-5-carbaldehyde (500 mg, 2.9 mmol, 1 equiv) followed by cyano acetic acid (668 mg, 5.9 mmol, 2 equiv) at room temperature. The reaction mixture was allowed to stir at 60° C. for 6 h in a screw cap bottle. Progress of the reaction was monitored by LCMS. After completion of the reaction, the reaction mixture was concentrated under reduced pressure, diluted with water (30 mL) and extracted with ethyl acetate (35 mL×2). The combined organic layer was dried over anhydrous sodium sulphate and concentrated under reduced pressure to obtain crude, which was purified by combi flash to afford the desired product. LCMS: 219 [M+H]⁺

Step—2: Synthesis of 8-benzyl-2-(methylthio)-7-oxo-7,8-dihydropyrido[2,3-d]pyrimidine-6-carbonitrile: To a stirred solution of obtain 2-(methylthio)-7-oxo-7,8-dihydropyrido[2,3-d]pyrimidine-6-carbonitrile (50 mg, 0.22 mmol, 1 equiv.) in N-methyl-2-pyrrolidone (2 mL) was added K₂CO₃ (61 mg, 0.44 mmol, 2 equiv) followed by benzyl bromide (58 mg, 0.44 mmol, 2 equiv.) at room temperature. The reaction mixture was stirred at 60° C. for 4 h in a screw cap bottle. Progress of the reaction was monitored by LCMS. After completion of the reaction, the reaction mixture was diluted with water (8 mL) and extracted with ethyl acetate (10 mL×2). The combined organic layer was washed with water (10 mL×2), dried over anhydrous sodium sulphate and concentrated under reduced pressure to obtain crude, which was purified by combi flash to afford desired product. LCMS: 309 [M+H]⁺

Step—3: Synthesis of 8-benzyl-2-(methylsulfinyl)-7-oxo-7,8-dihydropyrido[2,3-d]pyrimidine-6-carbonitrile: To a stirred solution of 8-benzyl-2-(methylthio)-7-oxo-7,8-dihydropyrido[2,3-d]pyrimidine-6-carbonitrile (200 mg, 0.65 mmol, 1.0 equiv.) in DCM was added m-CPBA (145 mg, 0.85 mmol, 1.3 equiv) at room temperature. The reaction mixture was allowed to stir at room temperature for 4 h. Progress of the reaction was monitored by LCMS. After completion of the reaction, the reaction mixture was diluted with sodium bicarbonate solution (15 mL) and extracted with DCM (15 mL×2). The combined organic layer was dried over anhydrous sodium sulphate and concentrated under reduced pressure to afford desired product. LCMS: 325 [M+H]⁺

Step—4: Synthesis of 8-benzyl-2-((4-(4-methylpiperazin-1-yl)phenyl)amino)-7-oxo-7,8-dihydropyrido[2,3-d]pyrimidine-6-carbonitrile: To a suspension of 8-benzyl-2-(methylsulfinyl)-7-oxo-7,8-dihydropyrido[2,3-d]pyrimidine-6-carbonitrile (40 mg, 0.12 mmol, 1 equiv) in toluene (3 mL), was added 4-(4-methylpiperazin-1-yl)aniline (26 mg, 0.13 mmol, 1.1 equiv). Resultant reaction mixture was stirred at 100° C. for 3 h. Reaction was monitored by LCMS. After completion of reaction, resultant mixture was concentrated under reduced pressure. Product was triturated from methanol to afford desired product. LCMS: 452 [M+H]⁺, ¹H NMR (400 MHz, Methanol-d4) δ 8.74 (s, 1H), 8.41 (s, 1H), 7.48 (s, 2H), 7.25 (d, J=8.0 Hz, 5H), 6.97 (d, J=8.6 Hz, 2H), 5.53 (s, 2H), 3.40 (s, 3H), 3.35 (s, 5H), 2.90 (s, 3H).

### Example—S67: Synthesis of 8-cyclopentyl-7-oxo-2-((4-(piperazin-1-yl)-3-(trifluoromethyl)phenyl)amino)-7,8-dihydropyrido[2,3-d]pyrimidine-6-carbonitrile (Compound No. 33)

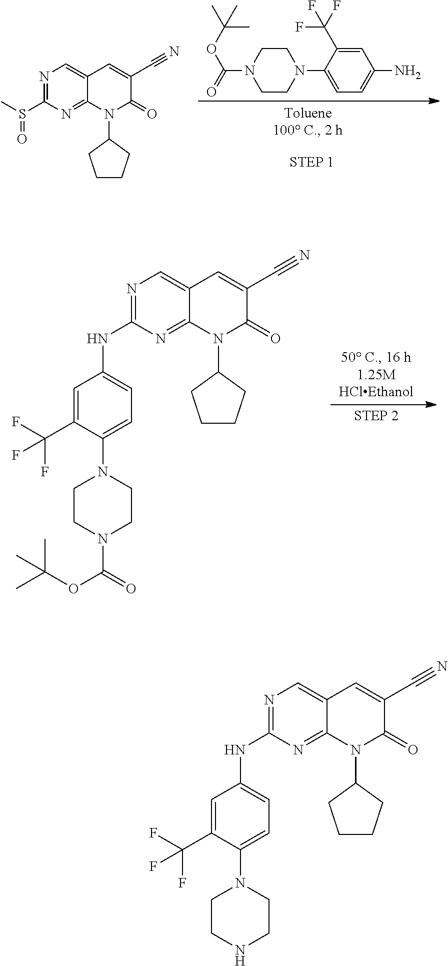

Step—1: Synthesis of tert-butyl 4-(4-((6-cyano-8-cyclopentyl-7-oxo-7,8-dihydropyrido[2,3-d]pyrimidin-2-yl)amino)-2-(trifluoromethyl)phenyl)piperazine-1-carboxylate. To a suspension of 8-cyclopentyl-2-(methylsulfinyl)-7-oxo-7,8-dihydropyrido[2,3-d]pyrimidine-6-carbonitrile (50 mg, 0.16 mmol, 1 equiv) in toluene (3 mL), was added tert-butyl 4-(4-amino-2-(trifluoromethyl)phenyl)piperazine-1-carboxylate (62 mg, 0.18 mmol, 1.1 equiv). Resultant reaction mixture was stirred at 100° C. for 3 h. Reaction was monitored by LCMS. After completion of reaction, reaction mixture was concentrated under reduced pressure. Product was triturated from methanol and dried to afford desired product. LCMS: 584 [M+H]⁺

Step—2: Synthesis of 8-cyclopentyl-7-oxo-2-((4-(piperazin-1-yl)-3-(trifluoromethyl)phenyl)amino)-7,8-dihydropyrido[2,3-d]pyrimidine-6-carbonitrile: To compound tert-butyl 4-(4-((6-cyano-8-cyclopentyl-7-oxo-7,8-dihydropyrido[2,3-d]pyrimidin-2-yl)amino)-2-(trifluoromethyl)phenyl)piperazine-1-carboxylate (27 mg, 0.05 mmol, 1.1 equiv) was added to the 1.25 M HCl in ethanol (3 mL). The resultant reaction mixture was stirred at 50° C. for 16 h. Reaction was monitored by LCMS. After completion of reaction, filtered the solid product and washed with diethyl ether and dried under reduced pressure to afford the desired product. LCMS: 484 [M+H]⁺; ¹H NMR (400 MHz, Methanol-d4) δ 8.81 (s, 1H), 8.39 (d, J=11.2 Hz, 2H), 7.76 (s, 1H), 7.56 (d, J=8.6 Hz, 1H), 6.00 (p, J=8.9 Hz, 1H), 3.36 (dd, J=6.4, 3.5 Hz, 4H), 3.19 (t, J=5.0 Hz, 4H), 2.26 (dq, J=14.9, 8.0 Hz, 2H), 2.08-1.99 (m, 2H), 1.90 (dq, J=10.5, 6.0 Hz, 2H), 1.67 (h, J=9.0, 8.3 Hz, 2H).

### Example—S68: Synthesis of 2-((4-(4-methylpiperazin-1-yl)phenyl)amino)-7-oxo-8-(1-phenylethyl)-7,8-dihydropyrido[2,3-d]pyrimidine-6-carbonitrile (Compound No. 35)

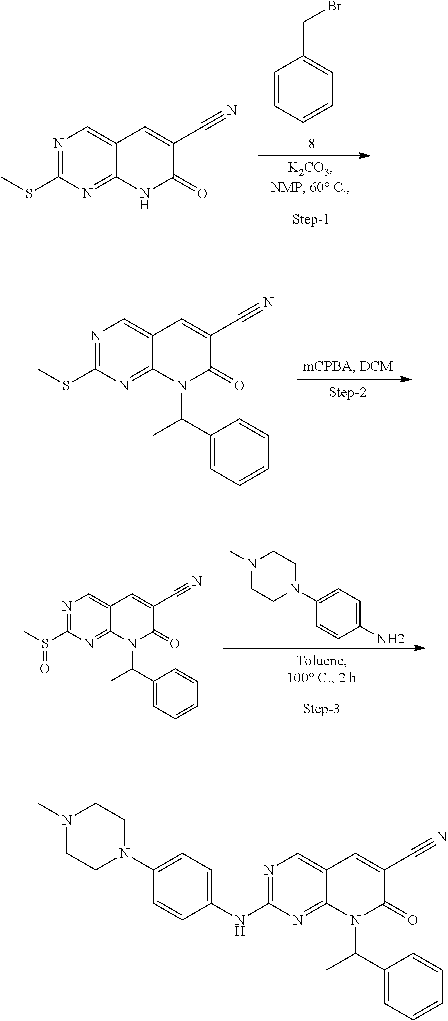

Step—1: Synthesis of 2-(methylthio)-7-oxo-8-(1-phenylethyl)-7,8-dihydropyrido[2,3-d]pyrimidine-6-carbonitrile: Toa stirred solution of obtain 2-(methylthio)-7-oxo-7,8-dihydropyrido[2,3-d]pyrimidine-6-carbonitrile (240 mg, 1.1 mmol, 1 equiv) in NMP (6 mL) was added K₂CO₃ (303 mg, 2.2 mmol, 2 equiv) followed by (1-bromoethyl)benzene (305 mg, 1.65 mmol, 1.5 equiv.) at room temperature. The reaction mixture was stirred at 70° C. for 4 h in a screw cap bottle. Progress of the reaction was monitored by LCMS. After completion of the reaction, the reaction mixture was diluted with water (8 mL) and extracted with ethyl acetate (10 mL×2). The combined organic layer was washed with water (10 mL×2), dried over anhydrous sodium sulphate and concentrated under reduced pressure to obtain crude, which was purified by combi flash to afford desired product. LCMS: 323 [M+H]⁺

Step—2: Synthesis of 2-(methylsulfinyl)-7-oxo-8-(1-phenylethyl)-7,8-dihydropyrido[2,3-d]pyrimidine-6-carbonitrile: To a stirred solution of 2-(methylthio)-7-oxo-8-(1-phenylethyl)-7,8-dihydropyrido[2,3-d]pyrimidine-6-carbonitrile (250 mg, 0.77 mmol, 1.0 equiv.) in DCM was added m-CPBA (294 mg, 1.71 mmol, 1.7 equiv) at room temperature. The reaction mixture was allowed to stir at room temperature for 4 h. Progress of the reaction was monitored by LCMS. After completion of the reaction, the reaction mixture was diluted with sodium bicarbonate solution (15 mL) and extracted with DCM (15 mL×2). The combined organic layer was dried over anhydrous sodium sulphate and concentrated under reduced pressure to afford desired product. LCMS: 339 [M+H]⁺

Step—3: Synthesis of 2-((4-(4-methylpiperazin-1-yl)phenyl)amino)-7-oxo-8-(1-phenylethyl)-7,8-dihydropyrido[2,3-d]pyrimidine-6-carbonitrile: To a suspension of 2-(methylsulfinyl)-7-oxo-8-(1-phenylethyl)-7,8-dihydropyrido[2,3-d]pyrimidine-6-carbonitrile (50 mg, 0.14 mmol, 1 equiv) in toluene (3 mL), was added 4-(4-methylpiperazin-1-yl)aniline (31 mg, 0.16 mmol, 1.1 equiv). Resultant reaction mixture was stirred at 100° C. for 3 h. Reaction was monitored by LCMS. After completion of reaction, resultant mixture was concentrated under reduced pressure. Product was purified by reverse phase HPLC to afford desired product. LCMS: 466 [M+H]⁺; ¹H NMR: (400 MHz, Methanol-d4) δ 8.73 (s, 1H), 8.49 (s, 1H), 8.38 (s, 1H), 7.31-7.17 (m, 6H), 6.95 (s, 2H), 3.24 (s, 4H), 2.82-2.75 (m, 4H), 2.47 (s, 3H), 1.94 (d, J=17.2 Hz, 3H).

### Example—S69: Synthesis of 8-cyclopentyl-2-((3-fluoro-4-(4-methylpiperazin-1-yl)phenyl)amino)-7-oxo-7,8-dihydropyrido[2,3-d]pyrimidine-6-carbonitrile (Compound No. 36)

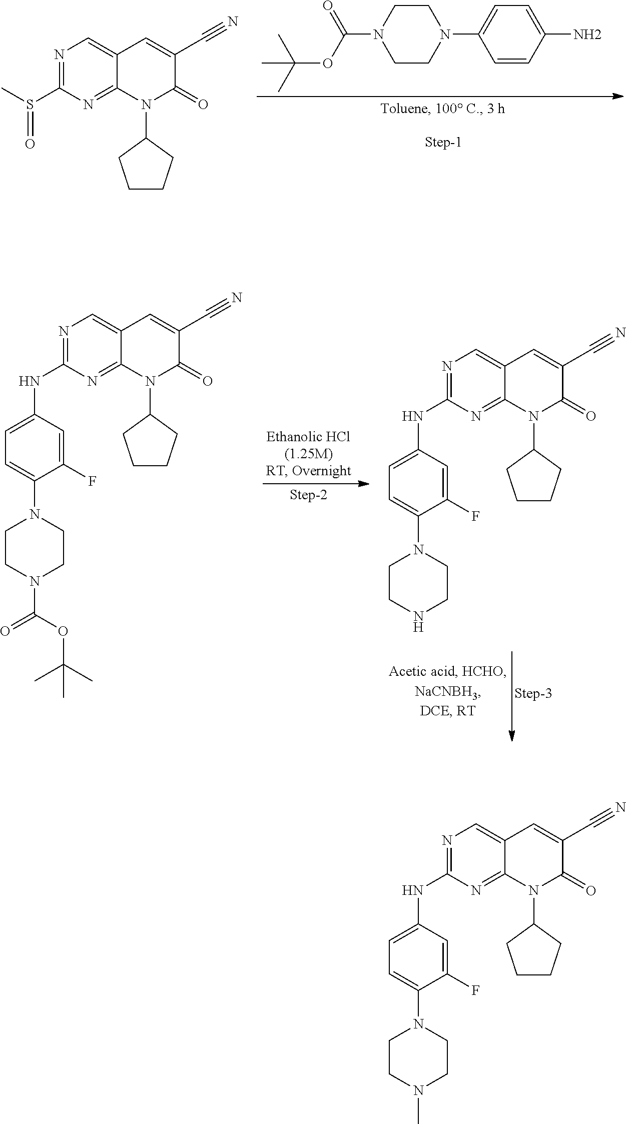 [SMILES: CS(=O)c1ncc2cc(C#N)c(=O)n(C3CCCC3)c2n1 ; CC(C)(C)OC(=O)N1CCN(c2ccc(N)cc2)CC1 ; CC(C)(C)OC(=O)N1CCN(c2ccc(Nc3ncc4cc(C#N)c(=O)n(C5CCCC5)c4n3)cc2F)CC1 ; N#Cc1cc2cnc(Nc3ccc(N4CCNCC4)c(F)c3)nc2n(C2CCCC2)c1=O ; CN1CCN(c2ccc(Nc3ncc4cc(C#N)c(=O)n(C5CCCC5)c4n3)cc2F)CC1]

Step—1: Synthesis of tert-butyl 4-(4-((6-cyano-8-cyclopentyl-7-oxo-7,8-dihydropyrido[2,3-d]pyrimidin-2-yl)amino)-2-fluorophenyl)piperazine-1-carboxylate: To a stirred solution of 8-cyclopentyl-2-(methylsulfinyl)-7-oxo-7, 8-dihydropyrido [2, 3-d]pyrimidine-6-carbonitrile (150 mg, 0.49 mmol, 1 equiv) in toluene (5 mL), was added tert-butyl 4-(4-amino-2-fluorophenyl) piperazine-1-carboxylate (161 mg, 0.54 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain desired product. LCMS: 534 [M+H]⁺

Step—2: Synthesis of 8-cyclopentyl-2-((3-fluoro-4-(piperazin-1-yl)phenyl)amino)-7-oxo-7,8-dihydropyrido[2,3-d]pyrimidine-6-carbonitrile. A solution of tert-butyl 4-(4-((6-cyano-8-cyclopentyl-7-oxo-7, 8-dihydropyrido [2, 3-d] pyrimidin-2-yl) amino)-2-fluorophenyl) piperazine-1-carboxylate (85 mg, 0.15 mmol, 1 equiv) in 1.25 M HCl in ethanol (5 mL) was allowed to stir for overnight at RT. Progress of the reaction was monitored by LCMS. After completion of the reaction, solvent was removed under reduced pressure to obtain desired product. LCMS: 434 [M+H]⁺

Step—3: Synthesis of 8-cyclopentyl-2-((3-fluoro-4-(4-methylpiperazin-1-yl)phenyl)amino)-7-oxo-7,8-dihydropyrido[2,3-d]pyrimidine-6-carbonitrile: To a stirred solution of 8-cyclopentyl-2-((3-fluoro-4-(piperazin-1-yl)phenyl)amino)-7-oxo-7,8-dihydropyrido[2,3-d]pyrimidine-6-carbonitrile (40 mg, 0.085 mmol, 1 equiv) in DCE (4 mL), was added HCHO in water (0.01 mL, 0.25 mmol, 3 equiv), acetic acid (0.02 mL, 0.42 mmol, 5 equiv). The reaction mixture was allowed to stir at RT for 1 h. The reaction mixture was cooled to 0° C. NaCNBH₃ (16 mg, 0.25 mmol, 3 equiv) was added to the reaction mixture and temperature was raised to RT. The reaction mixture was allowed to stir at RT for 1 h. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 448 [M+H]⁺; ¹H NMR (400 MHz, Methanol-d4) δ 8.76 (s, 1H), 8.37 (s, 1H), 7.70-7.61 (m, 1H), 7.29 (d, J=8.6 Hz, 1H), 7.07 (t, J=9.1 Hz, 1H), 5.95 (s, 1H), 3.21 (s, 4H), 2.99 (s, 4H), 2.62 (s, 3H), 2.30 (dq, J=14.6, 7.7 Hz, 2H), 1.99 (d, J=10.4 Hz, 2H), 1.88 (d, J=10.0 Hz, 2H), 1.68 (p, J=7.0, 5.6 Hz, 2H).

### Example—S70: Synthesis of 8-cyclopentyl-7-oxo-2-((4-(piperidin-4-yl)phenyl)amino)-7,8-dihydropyrido[2,3-d]pyrimidine-6-carbonitrile (Compound No. 37)

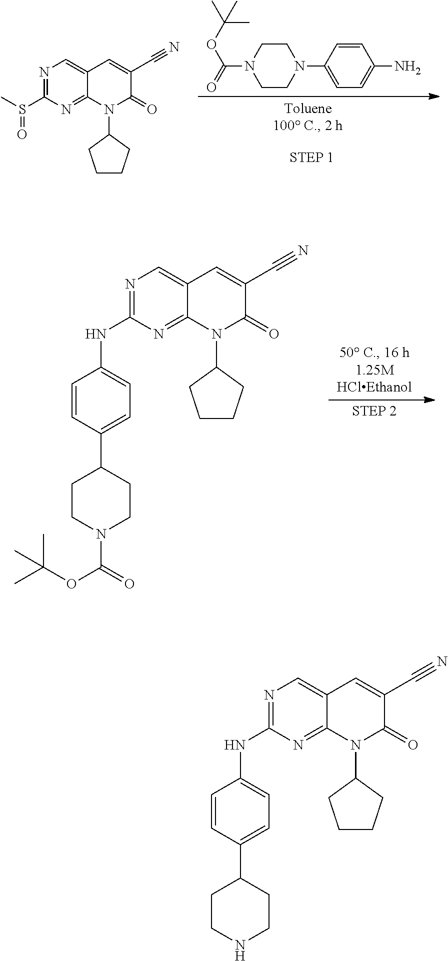

Step—1: Synthesis of tert-butyl 4-(4-((6-cyano-8-cyclopentyl-7-oxo-7,8-dihydropyrido[2,3-d]pyrimidin-2-yl)amino)phenyl)piperidine-1-carboxylate: To a suspension of 8-cyclopentyl-2-(methylsulfinyl)-7-oxo-7,8-dihydropyrido[2,3-d]pyrimidine-6-carbonitrile (50 mg, 0.17 mmol, 1 equiv) in toluene (3 mL), was added tert-butyl 4-(4-aminophenyl)piperidine-1-carboxylate (50 mg, 0.18 mmol, 1.1 equiv). Resultant reaction mixture was stirred at 100° C. for 3 h. Reaction was monitored by LCMS. After completion of reaction, reaction mixture was concentrated under reduced pressure. Product was purified by reverse phase HPLC to afford desired product. LCMS: 515 [M+H]⁺

Step—2: Synthesis of 8-cyclopentyl-7-oxo-2-((4-(piperidin-4-yl)phenyl)amino)-7,8-dihydropyrido[2,3-d]pyrimidine-6-carbonitrile: A suspension of tert-butyl 4-(4-((6-cyano-8-cyclopentyl-7-oxo-7,8-dihydropyrido[2,3-d]pyrimidin-2-yl)amino)-2-(trifluoromethyl)phenyl)piperazine-1-carboxylate (27 mg, 0.05 mmol, 1.1 equiv) in ethanolic HCl (3 mL) was stirred at 50° C. for 16 h. Reaction was monitored by LCMS. After completion of reaction, filtered the solid product and washed with diethyl ether and dried under reduced pressure to afford desired product. LCMS: 415 [M+H]⁺; ¹H NMR: (400 MHz, DMSO-d₆) δ 10.52-10.43 (m, 1H), 8.84 (s, 1H), 8.78 (d, J=10.6 Hz, 1H), 8.56 (d, 1H), 7.63 (d, J=8.1 Hz, 2H), 7.23 (d, J=8.1 Hz, 2H), 5.84-5.71 (m, 1H), 3.37 (d, J=12.4 Hz, 2H), 2.99 (q, J=12.0 Hz, 2H), 2.84 (t, J=12.2 Hz, 1H), 2.21 (dq, J=16.3, 8.5, 7.8 Hz, 2H), 2.09 (s, 2H), 1.98-1.74 (m, 6H), 1.56 (s, 2H).

### Example—S71: Synthesis of 7-oxo-2-((4-(piperazin-1-yl)phenyl)amino)-8-(1,2,3,4-tetrahydronaphthalen-1-yl)-7,8-dihydropyrido[2,3-d]pyrimidine-6-carbonitrile (Compound No. 39)

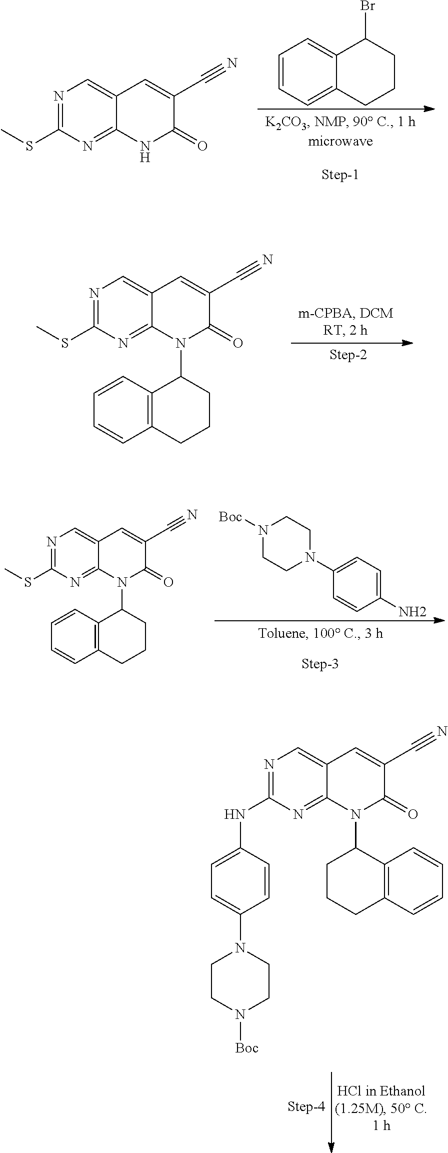 [SMILES: CSc1ncc2cc(C#N)c(=O)[nH]c2n1 ; BrC1CCCc2ccccc21 ; CSc1ncc2cc(C#N)c(=O)n(C3CCCc4ccccc43)c2n1 ; CSc1ncc2cc(C#N)c(=O)n(C3CCCc4ccccc43)c2n1 ; CC(C)(C)OC(=O)N1CCN(c2ccc(N)cc2)CC1 ; CC(C)(C)OC(=O)N1CCN(c2ccc(Nc3ncc4cc(C#N)c(=O)n(C5CCCc6ccccc65)c4n3)cc2)CC1]

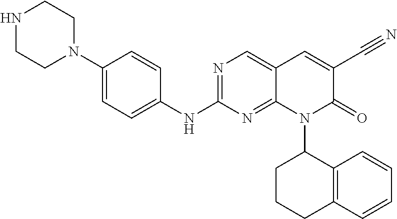 [SMILES: N#Cc1cc2cnc(Nc3ccc(N4CCNCC4)cc3)nc2n(C2CCCc3ccccc32)c1=O]

Step—1: Synthesis of 2-(methylthio)-7-oxo-8-(1,2,3,4-tetrahydronaphthalen-1-yl)-7,8-dihydropyrido[2,3-d]pyrimidine-6-carbonitrile: To a stirred solution of 2-(methylthio)-7-oxo-7, 8-dihydropyrido [2, 3-d] pyrimidine-6-carbonitrile (450 mg, 2.06 mmol, 1 equiv) in NMP (3 mL), was added K₂CO₃ (569 mg, 4.12 mmol, 2 equiv). The mixture was allowed to stir for 15 min. 1-bromo-1,2,3,4-tetrahydronaphthalene (867 mg, 4.12 mmol, 2 equiv) was added to above mixture and the mixture was allowed to stir for 1 h at 90° C. in microwave. Progress of the reaction was monitored by TLC and LCMS. After completion of the reaction, the reaction mixture was diluted with water (30 mL) and extracted with ethyl acetate (100 mL). Organic layer was washed with water (50 mL) and brine solution (100 mL). Organic layer was dried over anhydrous sodium sulphate and concentrated under reduced pressure to obtain crude compound, which was purified by normal phase combi flash to obtain desired product. LCMS: 349 [M+H]⁺

Step—2: Synthesis of 2-(methylsulfinyl)-7-oxo-8-(1,2,3,4-tetrahydronaphthalen-1-yl)-7,8-dihydropyrido[2,3-d]pyrimidine-6-carbonitrile: To a stirred solution of 2-(methylthio)-7-oxo-8-(1,2,3,4-tetrahydronaphthalen-1-yl)-7,8-dihydropyrido[2,3-d]pyrimidine-6-carbonitrile (160 mg, 0.45 mmol, 1 equiv) in DCM (5 mL), was added m-CPBA (103 mg, 0.59 mmol, 1.3 equiv) at RT. The mixture was allowed to stir for 2 h at RT. Progress of the reaction was monitored by TLC and LCMS. After completion of the reaction, the reaction mixture was diluted with DCM (30 mL) and washed with saturated solution of NaHCO₃(50 mL). Organic layer was dried over anhydrous sodium sulphate and concentrated under reduced pressure to obtain desired product. LCMS: 365.3 [M+H]⁺

Step—3: Synthesis of tert-butyl 4-(4-((6-cyano-7-oxo-8-(1,2,3,4-tetrahydronaphthalen-1-yl)-7,8-dihydropyrido[2,3-d]pyrimidin-2-yl)amino)phenyl)piperazine-1-carboxylate: To a stirred solution of 2-(methylsulfinyl)-7-oxo-8-(1,2,3,4-tetrahydronaphthalen-1-yl)-7,8-dihydropyrido[2,3-d]pyrimidine-6-carbonitrile (150 mg, 0.41 mmol, 1 equiv) in toluene (5 mL), was added tert-butyl 4-(4-aminophenyl)piperazine-1-carboxylate (125 mg, 0.45 mmol, 1.1 equiv). The resultant reaction mixture was stir at 100° C. for 3 h. Progress of the reaction was monitored by LCMS. After completion of the reaction, solid observed was filtered and dried under vacuum to obtain desired product. LCMS: 578 [M+H]⁺

Step—4: Synthesis of 7-oxo-2-((4-(piperazin-1-yl)phenyl)amino)-8-(1,2,3,4-tetrahydronaphthalen-1-yl)-7,8-dihydropyrido[2,3-d]pyrimidine-6-carbonitrile. A solution of tert-butyl 4-(4-((6-cyano-7-oxo-8-(1,2,3,4-tetrahydronaphthalen-1-yl)-7,8-dihydropyrido[2,3-d]pyrimidin-2-yl)amino)phenyl)piperazine-1-carboxylate (50 mg, 0.08 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain desired product. LCMS: 478[M+H]⁺; ¹H NMR 400 MHz, DMSO-d₆) δ 10.59 (s, 1H), 10.24 (s, 1H), 9.04-8.92 (m, 2H), 8.89 (s, 1H), 8.64 (s, 1H), 7.59 (d, J=8.6 Hz, 1H), 7.35 (d, J=8.2 Hz, 1H), 7.11 (qd, J=14.5, 7.1 Hz, 1H), 6.98 (dt, J=13.7, 7.6 Hz, 2H), 6.72 (q, J=14.0, 10.9 Hz, 2H), 3.28 (d, J=16.3 Hz, 9H), 2.97-2.84 (m, 1H), 2.79 (d, J=16.2 Hz, 1H), 2.17-2.08 (m, 2H), 1.91 (s, 1H).

### Example—S72: Synthesis of 8-cyclopentyl-2-((4-(1-methylpiperidin-4-yl)phenyl)amino)-7-oxo-7,8-dihydropyrido[2,3-d]pyrimidine-6-carbonitrile (Compound No. 40)

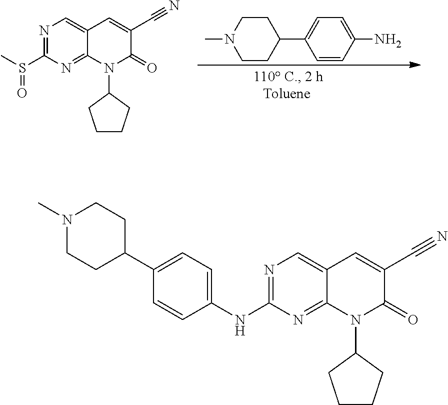 [SMILES: CS(=O)c1ncc2cc(C#N)c(=O)n(C3CCCC3)c2n1 ; CN1CCC(c2ccc(N)cc2)CC1 ; CN1CCC(c2ccc(Nc3ncc4cc(C#N)c(=O)n(C5CCCC5)c4n3)cc2)CC1]

To a suspension of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (2 mL), was added 4-(1-methylpiperidin-4-yl)aniline (68 mg, 0.36 mmol, 1.1 equiv). Resultant reaction mixture was stirred at 100° C. for 2 h. Reaction was monitored by LCMS. After completion of reaction, resultant mass was concentrated under reduced pressure. Product was triturated from methanol to afford desired product. LCMS: 429 (M+H); ¹H NMR: (400 MHz, CD₃OD) δ 8.79 (s, 1H), 8.39 (s, 1H), 7.6 (d, 2H), 7.7 (d, 1H), 5.97 (s, 1H), 2.8-2.6 (m, 5H), 2.3-2.2 (m, 2H), 2.1-1.8 (m, 7H), 1.7-1.6 (m, 2H).

### Example—S73: Synthesis of 8-cyclopentyl-2-((4-(2-(diethylamino)ethoxy)phenyl)amino)-7-oxo-7,8-dihydropyrido[2,3-d]pyrimidine-6-carbonitrile (Compound No. 41)

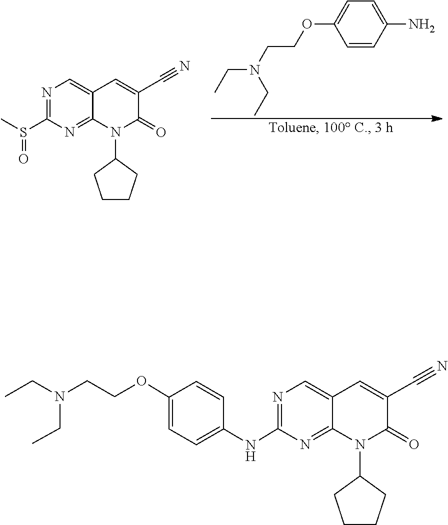 [SMILES: CS(=O)c1ncc2cc(C#N)c(=O)n(C3CCCC3)c2n1 ; CCN(CC)CCOc1ccc(N)cc1 ; CCN(CC)CCOc1ccc(Nc2ncc3cc(C#N)c(=O)n(C4CCCC4)c3n2)cc1]

To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 4-(2-(diethylamino)ethoxy)aniline (76 mg, 0.36 mmol, 1.1 equiv). The resultant reaction mixture was stir at 100° C. for 3 h. Progress of the reaction was monitored by LCMS. After completion of the reaction, solid observed was filtered and dried under vacuum to obtain desired product. LCMS: 447.5 [M+H]⁺; ¹H NMR (400 MHz, DMSO-d₆) δ 10.40 (s, 1H), 8.80 (s, 1H), 8.54 (s, 1H), 7.54 (d, J=8.4 Hz, 2H), 6.96 (d, J=8.6 Hz, 2H), 5.73 (dd, J=21.0, 12.3 Hz, 1H), 4.05 (t, J=6.0 Hz, 2H), 2.85 (t, J=6.0 Hz, 3H), 2.62 (q, J=7.2 Hz, 4H), 2.18 (q, J=9.1, 8.2 Hz, 2H), 1.78 (s, 3H), 1.56 (s, 2H), 1.00 (t, J=7.0 Hz, 6H).

### Example—S74: Synthesis of 8-(2,3-dihydro-1H-inden-1-yl)-7-oxo-2-((4-(piperazin-1-yl)phenyl)amino)-7,8-dihydropyrido[2,3-d]pyrimidine-6-carbonitrile (Compound No. 42)

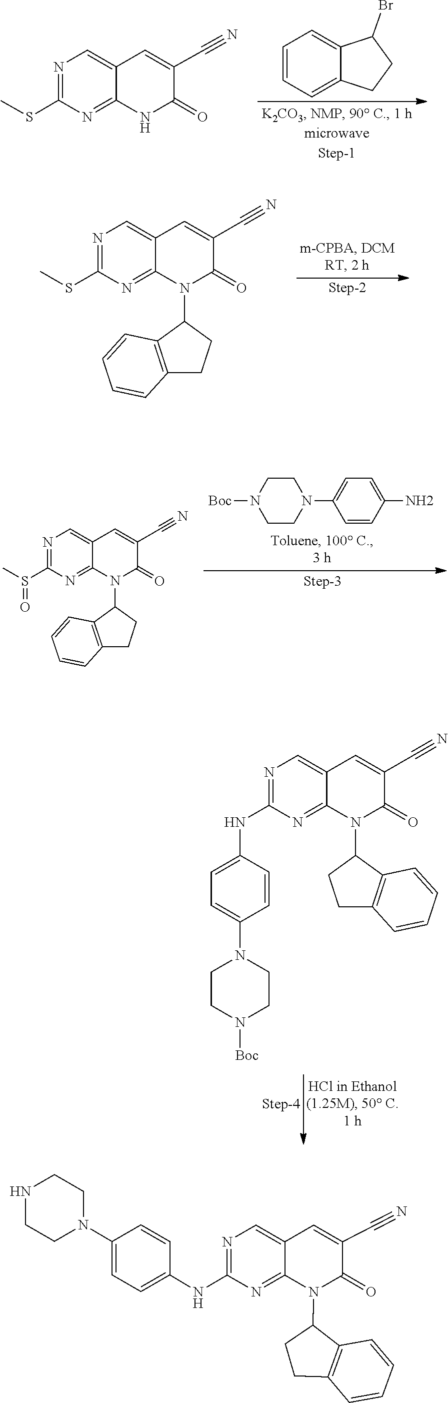

Step—1: Synthesis of 8-(2,3-dihydro-1H-inden-1-yl)-2-(methylthio)-7-oxo-7,8-dihydropyrido[2,3-d]pyrimidine-6-carbonitrile: To a stirred solution of 2-(methylthio)-7-oxo-7, 8-dihydropyrido [2, 3-d] pyrimidine-6-carbonitrile (400 mg, 1.83 mmol, 1 equiv) in NMP (4 mL), was added K₂CO₃ (505 mg, 3.66 mmol, 2 equiv). The mixture was allowed to stir for 15 min. 1-bromo-2, 3-dihydro-1H-indene (1076 mg, 3.66 mmol, 2 equiv) was added to above mixture and the mixture was allowed to stir for 1 h at 90° C. in microwave. Progress of the reaction was monitored by TLC and LCMS. After completion of the reaction, the reaction mixture was diluted with water (30 mL) and extracted with ethyl acetate (100 mL). Organic layer was washed with water (50 mL) and brine solution (100 mL). Organic layer was dried over anhydrous sodium sulphate and concentrated under reduced pressure to obtain crude compound, which was purified by normal phase combi flash to obtain desired product. LCMS: 335 [M+H]⁺

Step—2: Synthesis of 8-(2,3-dihydro-1H-inden-1-yl)-2-(methylsulfinyl)-7-oxo-7,8-dihydropyrido[2,3-d]pyrimidine-6-carbonitrile: To a stirred solution of 8-(2,3-dihydro-1H-inden-1-yl)-2-(methylthio)-7-oxo-7,8-dihydropyrido[2,3-d]pyrimidine-6-carbonitrile (120 mg, 0.35 mmol, 1 equiv) in DCM (5 mL), was added m-CPBA (81 mg, 0.46 mmol, 1.3 equiv) at RT. The mixture was allowed to stir for 2 h at RT. Progress of the reaction was monitored by TLC and LCMS. After completion of the reaction, the reaction mixture was diluted with DCM (30 mL) and washed with saturated solution of NaHCO₃(50 mL). Organic layer was dried over anhydrous sodium sulphate and concentrated under reduced pressure to obtain desired product. LCMS: 351 [M+H]⁺

Step—3: Synthesis of tert-butyl 4-(4-((6-cyano-8-(2,3-dihydro-1H-inden-1-yl)-7-oxo-7,8-dihydropyrido[2,3-d]pyrimidin-2-yl)amino)phenyl)piperazine-1-carboxylate. To a stirred solution of 8-(2, 3-dihydro-1H-inden-1-yl)-2-(methylsulfinyl)-7-oxo-7, 8-dihydropyrido [2, 3-d] pyrimidine-6-carbonitrile (110 mg, 0.31 mmol, 1 equiv) in toluene (5 mL), was added tert-butyl 4-(4-aminophenyl)piperazine-1-carboxylate (96 mg, 0.34 mmol, 1.1 equiv). The resultant reaction mixture was stir at 100° C. for 3 h. Progress of the reaction was monitored by LCMS. After completion of the reaction, solid observed was filtered and dried under vacuum to obtain desired product. LCMS: 564 [M+H]⁺

Step—4: Synthesis of 8-(2,3-dihydro-1H-inden-1-yl)-7-oxo-2-((4-(piperazin-1-yl)phenyl)amino)-7,8-dihydropyrido[2,3-d]pyrimidine-6-carbonitrile. A solution of tert-butyl 4-(4-((6-cyano-8-(2,3-dihydro-1H-inden-1-yl)-7-oxo-7,8-dihydropyrido[2,3-d]pyrimidin-2-yl)amino)phenyl)piperazine-1-carboxylate (40 mg, 0.07 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain desired product. LCMS: 464 [M+H]⁺; ¹H NMR (400 MHz, DMSO-d₆) δ 10.59 (s, 1H), 10.24 (s, 1H), 8.99-8.90 (m, 1H), 8.87 (s, 1H), 8.61 (s, 1H), 7.63 (s, 1H), 7.51-7.45 (m, 1H), 7.28 (d, J=6.9 Hz, 1H), 7.24-7.14 (m, 1H), 7.12-6.93 (m, 3H), 6.79 (d, J=12.8 Hz, 1H), 3.32-3.22 (m, 8H), 3.19 (s, 3H), 3.07-2.96 (m, 2H).

### Example—S75: Synthesis of 8-cyclopentyl-2-((3-fluoro-4-(piperidin-4-yl)phenyl)amino)-7-oxo-7,8-dihydropyrido[2,3-d]pyrimidine-6-carbonitrile (Compound No. 43)

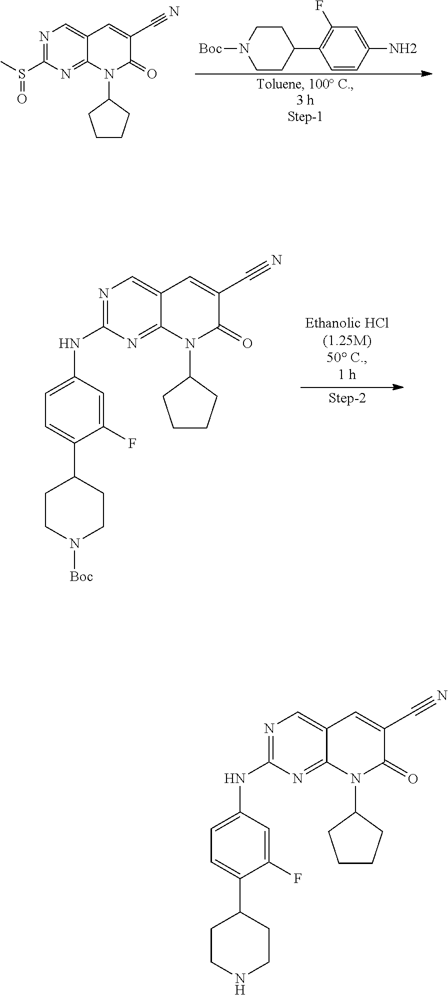 [SMILES: CS(=O)c1ncc2cc(C#N)c(=O)n(C3CCCC3)c2n1 ; CC(C)(C)OC(=O)N1CCC(c2ccc(N)cc2F)CC1 ; CC(C)(C)OC(=O)N1CCC(c2ccc(Nc3ncc4cc(C#N)c(=O)n(C5CCCC5)c4n3)cc2F)CC1 ; N#Cc1cc2cnc(Nc3ccc(C4CCNCC4)c(F)c3)nc2n(C2CCCC2)c1=O]

Step—1: Synthesis tert-butyl 4-(4-((6-cyano-8-cyclopentyl-7-oxo-7,8-dihydropyrido[2,3-d]pyrimidin-2-yl)amino)-2-fluorophenyl)piperidine-1-carboxylate: To a stirred solution of 8-cyclopentyl-2-(methylsulfinyl)-7-oxo-7, 8-dihydropyrido [2, 3-d]pyrimidine-6-carbonitrile (150 mg, 0.49 mmol, 1 equiv) in toluene (5 mL), was added tert-butyl 4-(4-amino-2-fluorophenyl) piperidine-1-carboxylate (160 mg, 0.54 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain desired product. LCMS: 533 [M+H]⁺

Step—2: Synthesis of 8-cyclopentyl-2-((3-fluoro-4-(piperidin-4-yl)phenyl)amino)-7-oxo-7,8-dihydropyrido[2,3-d]pyrimidine-6-carbonitrile: A solution of tert-butyl 4-(4-((6-cyano-8-cyclopentyl-7-oxo-7, 8-dihydropyrido [2, 3-d] pyrimidin-2-yl) amino)-2-fluorophenyl) piperidine-1-carboxylate (35 mg, 0.06 mmol, 1 equiv) in 1.25 M HCl in ethanol (5 mL) was allowed to stir for overnight at RT. Progress of the reaction was monitored by LCMS. After completion of the reaction, solvent was removed under reduced pressure to obtain desired product. LCMS: 433 [M+H]⁺; ¹H NMR (400 MHz, DMSO-d₆) δ 10.67 (s, 1H), 8.86 (d, J=17.1 Hz, 2H), 8.61 (s, 1H), 7.75-7.66 (m, 1H), 7.43 (d, J=8.5 Hz, 1H), 7.26 (t, J=8.5 Hz, 1H), 5.85-5.75 (m, 1H), 3.06 (dp, J=30.6, 9.9, 8.8 Hz, 4H), 2.22 (dq, J=14.3, 8.3 Hz, 2H), 2.01-1.75 (m, 9H), 1.60 (dt, J=11.1, 6.4 Hz, 2H).

### Example—S76: Synthesis of 8-cyclopentyl-2-((4-(4-(dimethylamino)piperidin-1-yl)-3-fluorophenyl)amino)-7-oxo-7,8-dihydropyrido[2,3-d]pyrimidine-6-carbonitrile (Compound No. 44)

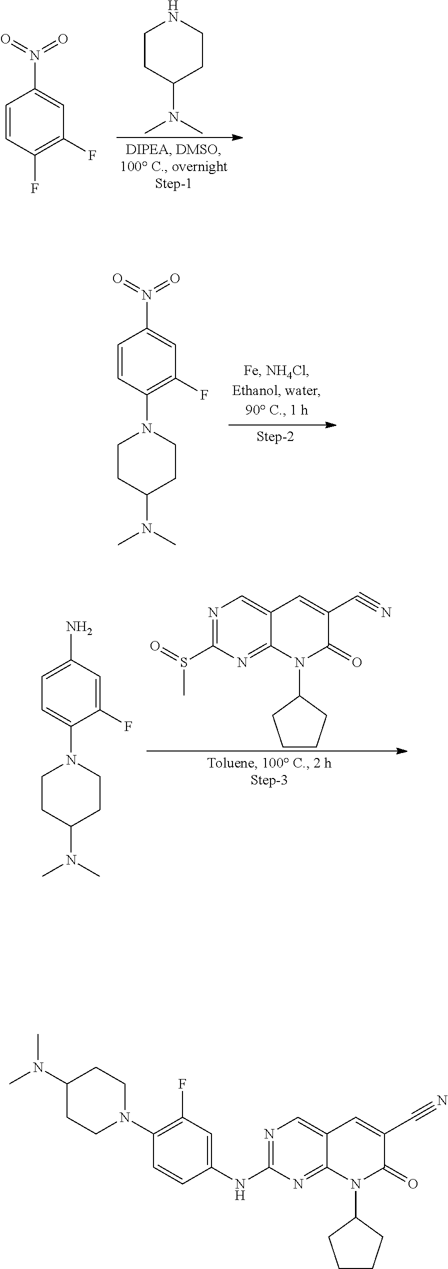

Step—1: Synthesis of 1-(2-fluoro-4-nitrophenyl)-N,N-dimethylpiperidin-4-amine: To a stirred solution of 1, 2-difluoro-4-nitrobenzene (400 mg, 2.5 mmol, 1 equiv) in DMSO (10 mL), was added DIPEA (1.7 mL, 10 mmol, 4 equiv) and N,N-dimethylpiperidin-4-amine (556 mg, 2.76 mmol, 1.1 equiv). The resultant reaction mixture was allowed to stir at 100° C. for overnight. Progress of the reaction was monitored by TLC and LCMS. After completion of the reaction, the mixture was diluted with ice water (10 mL), solid observed was filtered and dried under vacuum to obtain desired product: LCMS: 268 [M+H]⁺

Step—2: Synthesis of 1-(4-amino-2-fluorophenyl)-N,N-dimethylpiperidin-4-amine: To a stirred solution of 1-(2-fluoro-4-nitrophenyl)-N,N-dimethylpiperidin-4-amine (500 mg, 1.87 mmol, 1 equiv) in ethanol (8 mL), water (2 mL), was added iron powder (315 mg, 5.61 mmol, 3 equiv) and ammonium chloride (202 mg, 3.74 mmol, 2 equiv). The resultant reaction mixture was allowed to stir at 90° C. for 1 h. Progress of the reaction was monitored by TLC and LCMS. After completion of the reaction, the mixture was diluted with water (30 mL) and extracted with EtOAc (100 mL). Organic layer was washed with water (50 mL) and brine (50 mL). Organic layer was dried over anhydrous sodium sulphate and concentrated under reduced pressure to obtain desired product. LCMS: 238 [M+H]⁺

Step—3: Synthesis 8-cyclopentyl-2-((4-(4-(dimethylamino)piperidin-1-yl)-3-fluorophenyl)amino)-7-oxo-7,8-dihydropyrido[2,3-d]pyrimidine-6-carbonitrile: To a stirred solution of 8-cyclopentyl-2-(methylsulfinyl)-7-oxo-7, 8-dihydropyrido [2, 3-d] pyrimidine-6-carbonitrile (150 mg, 0.49 mmol, 1 equiv) in toluene (5 mL), was added 1-(4-amino-2-fluorophenyl)-N, N-dimethylpiperidin-4-amine (129 mg, 0.54 mmol, 1.1 equiv). The resultant reaction mixture was allowed to stir at 100° C. for 2 h. Progress of the reaction was monitored by LCMS. After completion of the reaction, solid observed was filtered and dried under vacuum to obtain desired product. LCMS: 476 [M+H]⁺; ¹H NMR: (400 MHz, DMSO-d₆) δ 10.62 (s, 1H), 8.84 (s, 1H), 8.59 (s, 1H), 7.66 (d, J=14.6 Hz, 1H), 7.33 (s, 1H), 7.12-7.04 (m, 1H), 5.74 (s, 1H), 3.44 (d, J=11.7 Hz, 2H), 3.25 (s, 2H), 2.76 (s, 6H), 2.68 (t, J=11.1 Hz, 2H), 2.26-2.17 (m, 2H), 2.13-2.06 (m, 2H), 1.93 (s, 2H), 1.79 (q, J=11.1, 9.3 Hz, 3H), 1.61-1.56 (m, 2H).

### Example—S77: Synthesis of 8-cyclopentyl-2-((3-fluoro-4-(1-methylpiperidin-4-yl)phenyl)amino)-7-oxo-7,8-dihydropyrido[2,3-d]pyrimidine-6-carbonitrile (Compound No. 46)

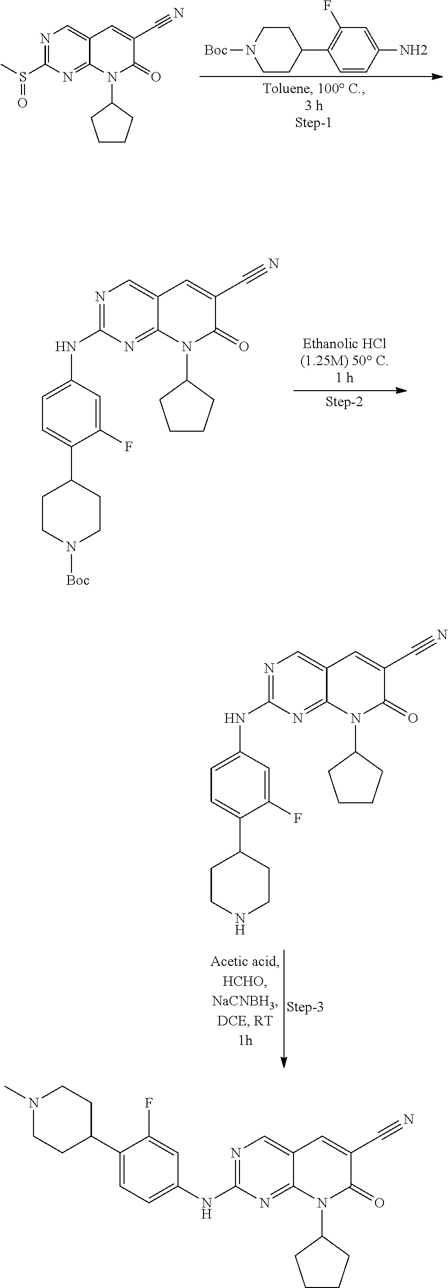 [SMILES: CS(=O)c1ncc2cc(C#N)c(=O)n(C3CCCC3)c2n1 ; CC(C)(C)OC(=O)N1CCC(c2ccc(N)cc2F)CC1 ; CC(C)(C)OC(=O)N1CCC(c2ccc(Nc3ncc4cc(C#N)c(=O)n(C5CCCC5)c4n3)cc2F)CC1 ; N#Cc1cc2cnc(Nc3ccc(C4CCNCC4)c(F)c3)nc2n(C2CCCC2)c1=O ; CN1CCC(c2ccc(Nc3ncc4cc(C#N)c(=O)n(C5CCCC5)c4n3)cc2F)CC1]

Step—1: Synthesis of tert-butyl 4-(4-((6-cyano-8-cyclopentyl-7-oxo-7,8-dihydropyrido[2,3-d]pyrimidin-2-yl)amino)-2-fluorophenyl)piperidine-1-carboxylate: To a stirred solution of 8-cyclopentyl-2-(methylsulfinyl)-7-oxo-7, 8-dihydropyrido [2, 3-d]pyrimidine-6-carbonitrile (150 mg, 0.49 mmol, 1 equiv) in toluene (5 mL), was added tert-butyl 4-(4-amino-2-fluorophenyl)piperidine-1-carboxylate (160 mg, 0.54 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain desired product. LCMS: 533 [M+H]⁺

Step—2: Synthesis of 8-cyclopentyl-2-((3-fluoro-4-(piperidin-4-yl)phenyl)amino)-7-oxo-7,8-dihydropyrido[2,3-d]pyrimidine-6-carbonitrile: A solution tert-butyl 4-(4-((6-cyano-8-cyclopentyl-7-oxo-7, 8-dihydropyrido [2, 3-d] pyrimidin-2-yl) amino)-2-fluorophenyl) piperidine-1-carboxylate (50 mg, 0.09 mmol, 1 equiv) in 1.25 M HCl in ethanol (5 mL) was allowed to stir for overnight at RT. Progress of the reaction was monitored by LCMS. After completion of the reaction, solvent was removed under reduced pressure to obtain desired product. LCMS: 433 [M+H]⁺

Step—3: Synthesis of 8-cyclopentyl-2-((3-fluoro-4-(1-methylpiperidin-4-yl)phenyl)amino)-7-oxo-7,8-dihydropyrido[2,3-d]pyrimidine-6-carbonitrile: To a stirred solution of 8-cyclopentyl-2-((3-fluoro-4-(1-methylpiperidin-4-yl)phenyl)amino)-7-oxo-7,8-dihydropyrido[2,3-d]pyrimidine-6-carbonitrile (40 mg, 0.09 mmol, 1 equiv) in DCE (5 mL), was added HCHO in 40% water (8.3 mg, 0.27 mmol, 3 equiv), acetic acid (0.02 mL, 0.45 mmol, 5 equiv). The reaction mixture was allowed to stir at RT for 1 h. The reaction mixture was cooled to 0° C. NaCNBH₃ (17 mg, 0.27 mmol, 3 equiv) was added to above mixture and temperature was raised to RT. The reaction mixture was allowed to stir at RT for 1 h. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to afford desired product. LCMS: 447 [M+H]⁺; ¹H NMR (400 MHz, DMSO-d₆) δ 10.62 (s, 1H), 8.87 (s, 1H), 8.60 (s, 1H), 7.65 (d, J=13.1 Hz, 1H), 7.35 (dd, J=25.2, 8.5 Hz, 2H), 5.78 (d, J=16.4 Hz, 1H), 3.19 (s, 3H), 2.88 (d, J=11.0 Hz, 1H), 2.42 (s, 6H), 2.20 (s, 1H), 1.86-1.69 (q, J=4.2 Hz, 7H), 1.60 (p, J=6.8, 5.7 Hz, 2H).

### Example—S78: Synthesis of 8-cyclopentyl-2-((3-fluoro-4-(4-hydroxypiperidin-1-yl)phenyl)amino)-7-oxo-7,8-dihydropyrido[2,3-d]pyrimidine-6-carbonitrile (Compound No. 49)

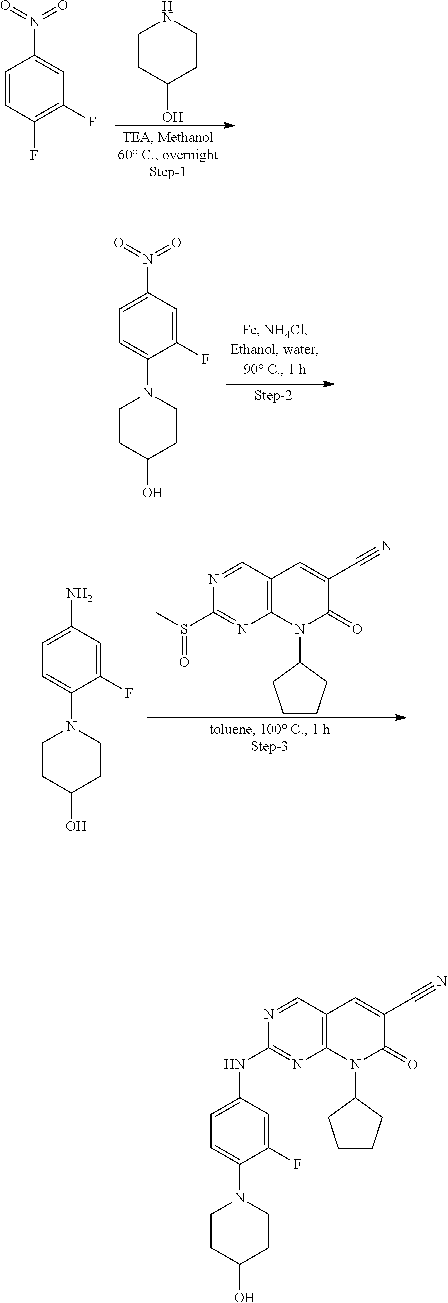 [SMILES: O=[N+]([O-])c1ccc(F)c(F)c1 ; OC1CCNCC1 ; O=[N+]([O-])c1ccc(N2CCC(O)CC2)c(F)c1 ; Nc1ccc(N2CCC(O)CC2)c(F)c1 ; CS(=O)c1ncc2cc(C#N)c(=O)n(C3CCCC3)c2n1 ; N#Cc1cc2cnc(Nc3ccc(N4CCC(O)CC4)c(F)c3)nc2n(C2CCCC2)c1=O]

Step—1: Synthesis of 1-(2-fluoro-4-nitrophenyl)piperidin-4-ol: To a stirred solution of 1, 2-difluoro-4-nitrobenzene (1000 mg, 6.28 mmol, 1 equiv) in methanol (15 mL), was added TEA (1.7 mL, 9.43 mmol, 2 equiv) and piperidin-4-ol (953 mg, 12.5 mmol, 1.5 equiv). The resultant reaction mixture was allowed to stir at 60° C. for overnight. Progress of the reaction was monitored by LCMS. After completion of the reaction, diluted with water (100 mL), solid observed was filtered and dried under vacuum to obtain desired product. LCMS: 241 [M+H]⁺

Step—2: Synthesis of 1-(4-amino-2-fluorophenyl)piperidin-4-ol: To a stirred solution of 1-(2-fluoro-4-nitrophenyl)piperidin-4-ol (500 mg, 2.08 mmol, 1 equiv) in ethanol (8 mL), water (2 mL), was added iron powder (350 mg, 6.25 mmol, 3 equiv) and ammonium chloride (225 mg, 4.16 mmol, 2 equiv). The resultant reaction mixture was allowed to stir at 90° C. for 1 h. Progress of the reaction was monitored by TLC and LCMS. After completion of the reaction, the mixture was passes through celite bed and the filtrate was concentrated under reduced pressure to obtain desired product. LCMS: 211 [M+H]⁺

Step—3: Synthesis of 8-cyclopentyl-2-((3-fluoro-4-(4-hydroxypiperidin-1-yl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 1-(4-amino-2-fluorophenyl) piperidin-4-ol (77 mg, 0.36 mmol, 1.1 equiv). The resultant reaction mixture was stir at 100° C. for 1 h. Progress of the reaction was monitored by LCMS. After completion of the reaction, solvent was removed under reduced pressure to obtain crude which was purified by trituration in methanol to obtain desired product. LCMS: 448 [M+H]⁺; ¹H NMR: (400 MHz, DMSO-d₆) δ 10.55 (s, 1H), 8.83 (s, 1H), 8.57 (s, 1H), 7.61 (d, J=14.8 Hz, 1H), 7.37-7.20 (m, 1H), 7.04 (t, J=9.1 Hz, 1H), 5.84-5.69 (m, 1H), 4.68 (d, J=4.0 Hz, 1H), 3.60 (s, 1H), 3.19 (dd, J=16.2, 8.2 Hz, 2H), 2.74 (t, J=10.5 Hz, 2H), 2.21 (p, J=8.3 Hz, 2H), 1.83 (t, J=14.4 Hz, 6H), 1.65-1.47 (m, 4H).

### Example—S79: Synthesis of 8-cyclopentyl-7-oxo-2-((1-(piperidin-4-yl)-1H-pyrazol-4-yl) amino)-7, 8-dihydropyrido [2, 3-d] pyrimidine-6-carbonitrile (Compound No. 50)

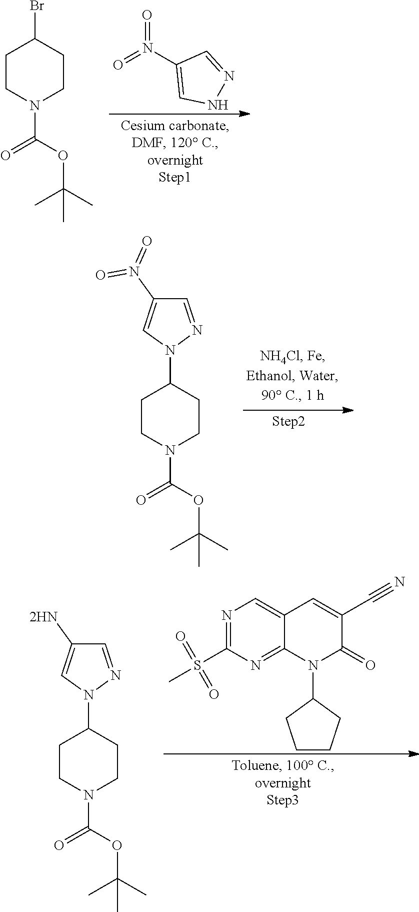

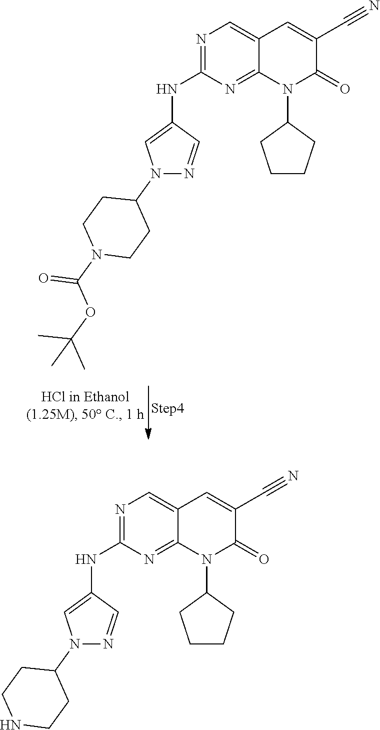 [SMILES: CC(C)(C)OC(=O)N1CCC(n2cc(Nc3ncc4cc(C#N)c(=O)n(C5CCCC5)c4n3)cn2)CC1 ; N#Cc1cc2cnc(Nc3cnn(C4CCNCC4)c3)nc2n(C2CCCC2)c1=O]

Step—1: Synthesis of tert-butyl 4-(4-nitro-1H-pyrazol-1-yl)piperidine-1-carboxylate: To a stirred solution of tert-butyl 4-bromopiperidine-1-carboxylate (1000 mg, 8.7 mmol, 1 equiv) in DMF (15 mL), was added Cs₂CO₃ (5672 mg, 17.4 mmol, 1 equiv) and 4-nitro-1H-pyrazole (2693 mg, 10.2 mmol, 1.2 equiv). The resultant reaction mixture was allowed to stir at 120° C. for overnight. Progress of the reaction was monitored by TLC and LCMS. After completion of the reaction, diluted with water (100 mL) and extracted with ethyl acetate (150 mL×2). Organic layer was washed with water (100 mL) and brine solution (100 mL). Organic layer was dried over anhydrous sodium sulphate and concentrated under reduced pressure to obtain crude, which was purified by normal phase combi flash to obtain desired product. LCMS: 241 [M-t-But+H]⁺

Step—2: Synthesis of tert-butyl 4-(4-amino-1H-pyrazol-1-yl)piperidine-1-carboxylate: To a stirred solution of tert-butyl 4-(4-nitro-1H-pyrazol-1-yl)piperidine-1-carboxylate (1000 mg, 3.37 mmol, 1 equiv) in ethanol (10 mL), water (3 mL), was added iron powder (566 mg, 10.11 mmol, 3 equiv) and ammonium chloride (364 mg, 6.74 mmol, 2 equiv). The resultant reaction mixture was allowed to stir at 90° C. for 1 h. Progress of the reaction was monitored by TLC and LCMS. After completion of the reaction, the mixture was passes through celite bed and the filtrate was concentrated under reduced pressure to obtain desired product. LCMS: 267 [M+H]⁺

Step—3: Synthesis of tert-butyl 4-(4-((6-cyano-8-cyclopentyl-7-oxo-7,8-dihydropyrido[2,3-d]pyrimidin-2-yl)amino)-1H-pyrazol-1-yl)piperidine-1-carboxylate: To a stirred solution of 8-cyclopentyl-2-(methylsulfonyl)-7-oxo-7, 8-dihydropyrido [2, 3-d]pyrimidine-6-carbonitrile (100 mg, 0.33 mmol, 1 equiv) in toluene (5 mL), was added tert-butyl 4-(4-amino-1H-pyrazol-1-yl) piperidine-1-carboxylate (77 mg, 0.36 mmol, 1.1 equiv). The resultant reaction mixture was stir at 100° C. for 1 h. Progress of the reaction was monitored by LCMS. After completion of the reaction, solvent was removed under reduced pressure to obtain crude which was purified by recrystallization with methanol to obtain desired product. LCMS: 505 [M+H]⁺

Step—4: Synthesis of 8-cyclopentyl-7-oxo-2-((1-(piperidin-4-yl)-1H-pyrazol-4-yl)amino)-7,8-dihydropyrido[2,3-d]pyrimidine-6-carbonitrile: Tert-butyl 4-(4-((6-cyano-8-cyclopentyl-7-oxo-7,8-dihydropyrido [2,3-d] pyrimidin-2-yl) amino)-1H-pyrazol-1-yl) piperidine-1-carboxylate (75 mg, 0.14 mmol, 1 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure and the residue was dried under lyophilizer to obtain desired product. LCMS: 405 [M+H]⁺; ¹H NMR: (400 MHz, DMSO-d6) δ 10.27 (s, 1H), 8.79 (s, 1H), 8.49 (s, 1H), 7.91 (s, 1H), 7.69 (s, 1H), 5.82 (dt, J=16.9, 8.2 Hz, 1H), 4.50 (t, J=7.5 Hz, 1H), 3.38 (d, J=12.9 Hz, 2H), 3.09 (d, J=11.8 Hz, 2H), 2.23-2.30 (m, 6H), 1.97 (s, 2H), 1.83 (t, J=10.7 Hz, 2H), 1.55-1.70 (m, 2H).

### Example—S80: Synthesis of 8-cyclopentyl-2-((1-(4-hydroxycyclohexyl)-1H-pyrazol-4-yl)amino)-7-oxo-7,8-dihydropyrido[2,3-d]pyrimidine-6-carbonitrile (Compound No. 51)

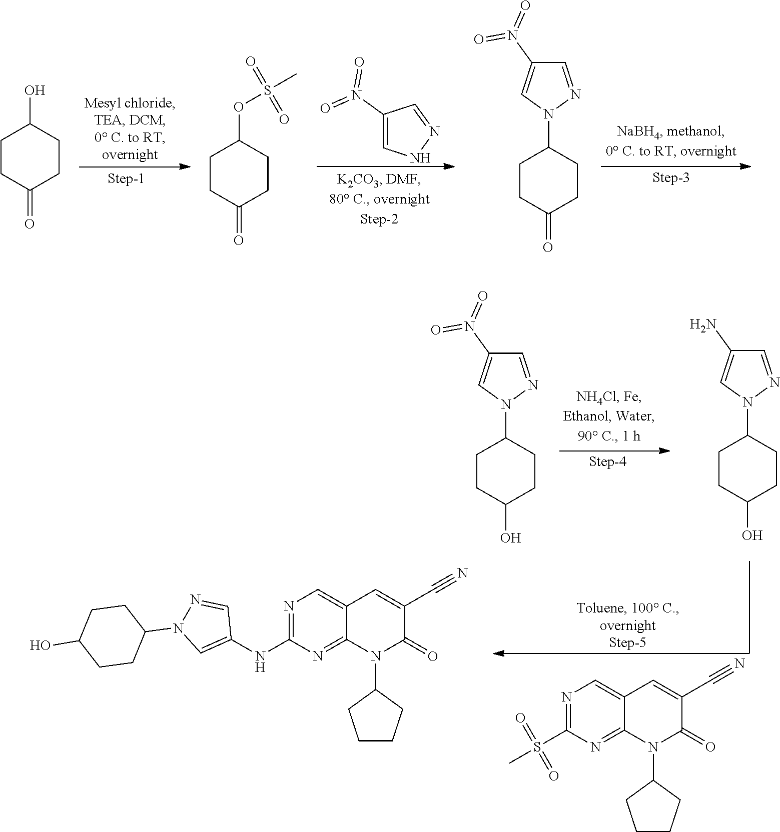

Step—1: Synthesis of 4-oxocyclohexyl methanesulfonate: To a stirred solution of 4-hydroxycyclohexan-1-one (1000 mg, 8.7 mmol, 1 equiv) in DCM (15 mL), was added TEA (1.2 mL, 8.7 mmol, 1 equiv). Reaction mixture was cooled to 0° C., followed by the addition of mesyl chloride (0.7 mL, 8.7 mmol, 1 equiv). Temperature was raised to RT and the resultant reaction mixture was allowed to stir for overnight. Progress of the reaction was monitored by LCMS. After completion of the reaction, diluted with water (50 mL) and extracted with DCM (100 mL×2). Organic layer was washed with water (100 mL) and brine solution (100 mL). Organic layer was dried over anhydrous sodium sulphate and concentrated under reduced pressure to obtain the desired product. LCMS: 193 [M+H]⁺

Step—2: Synthesis of 4-(4-nitro-1H-pyrazol-1-yl)cyclohexan-1-one: To a stirred solution of 4-oxocyclohexyl methanesulfonate (600 mg, 5.3 mmol, 1 equiv) in DMF (10 mL), was added K₂CO₃ (1463 mg, 10.6 mmol, 2 equiv) and 4-nitro-1H-pyrazole (1223 mg, 6.37 mmol, 1.2 equiv). The resultant reaction mixture was allowed to stir at 80° C. for overnight. Progress of the reaction was monitored by LCMS. After completion of the reaction, diluted with water (50 mL) and extracted with EtOAc (100 mL×2). Organic layer was washed with water (100 mL) and brine solution (100 mL). Organic layer was dried over anhydrous sodium sulphate and concentrated under reduced pressure to obtain crude, which was purified by normal phase Combi flash to obtain desired product. LCMS: 210 [M+H]⁺

Step—3: Synthesis of 4-(4-nitro-1H-pyrazol-1-yl)cyclohexan-1-ol: To a stirred solution of 4-(4-nitro-1H-pyrazol-1-yl) cyclohexan-1-one (400 mg, 1.91 mmol, 1 equiv) in methanol (10 mL), was added NaBH₄ (145 mg, 3.82 mmol, 2 equiv) at 0° C. The temperature was raised to RT and the resultant reaction mixture was allowed to stir at RT for overnight. Progress of the reaction was monitored by TLC and LCMS. After completion of the reaction, solvent was removed under reduced pressure, residue obtain was diluted with water (50 mL) and extracted with ethyl acetate (150 mL). Organic layer was washed with water (50 mL) and brine solution (50 mL). Organic layer was dried over anhydrous sodium sulphate and concentrated under reduced pressure to obtain crude, which was purified by normal phase Combi flash to obtain the desired product. LCMS: 212 [M+H]⁺

Step—4: Synthesis of 4-(4-amino-1H-pyrazol-1-yl)cyclohexan-1-ol: To a stirred solution of 4-(4-nitro-1H-pyrazol-1-yl)cyclohexan-1-ol (300 mg, 1.42 mmol, 1 equiv) in ethanol (8 mL), water (2 mL), was added iron powder (239 mg, 4.26 mmol, 3 equiv) and ammonium chloride (154 mg, 2.84 mmol, 2 equiv). The resultant reaction mixture was allowed to stir at 90° C. for 1 h. Progress of the reaction was monitored by TLC and LCMS. After completion of the reaction, the mixture was passes through celite bed and the filtrate was concentrated under reduced pressure to obtain the desired product. LCMS: 182 [M+H]⁺

Step—5: Synthesis of 8-cyclopentyl-2-((1-(4-hydroxycyclohexyl)-1H-pyrazol-4-yl)amino)-7-oxo-7,8-dihydropyrido[2,3-d]pyrimidine-6-carbonitrile: To a stirred solution of 8-cyclopentyl-2-(methylsulfonyl)-7-oxo-7, 8-dihydropyrido [2, 3-d] pyrimidine-6-carbonitrile (100 mg, 0.33 mmol, 1 equiv) in toluene (5 mL), was added 4-(4-amino-1H-pyrazol-1-yl)cyclohexan-1-ol (63 mg, 0.36 mmol, 1.1 equiv). The resultant reaction mixture was stirred at 100° C. for 1 h. Progress of the reaction was monitored by LCMS. After completion of the reaction, solvent was removed under reduced pressure to obtain crude which was purified by trituration in methanol to obtain desired product. LCMS: 420 [M+H]⁺; ¹H NMR: (400 MHz, DMSO-d₆) δ 10.41 (d, J=108.4 Hz, 1H), 8.80 (d, J=27.7 Hz, 1H), 8.54 (d, J=8.1 Hz, 1H), 7.95 (d, J=11.2 Hz, 1H), 7.56 (d, J=11.0 Hz, 1H), 5.83 (h, J=9.0, 8.5 Hz, 1H), 4.67 (dd, J=18.5, 4.5 Hz, 1H), 4.18-4.06 (m, 1H), 3.52-3.42 (m, 2H), 2.25-2.13 (m, 2H), 2.11-1.87 (m, 6H), 1.80-1.67 (m, 3H), 1.60 (s, 2H), 1.36 (q, J=11.5, 10.7 Hz, 2H).

### Example—S81: Synthesis of 8-cyclopentyl-2-((3-fluoro-4-(hexahydropyrrolo[1,2-a]pyrazin-2(1H)-yl)phenyl)amino)-7-oxo-7,8-dihydropyrido[2,3-d]pyrimidine-6-carbonitrile (Compound No. 55)

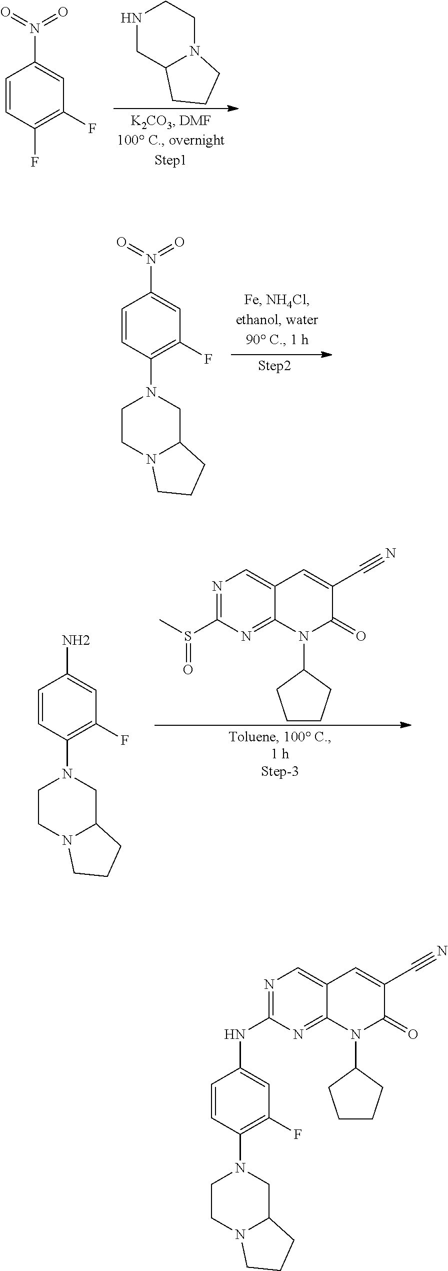 [SMILES: O=[N+]([O-])c1ccc(F)c(F)c1 ; C1CC2CNCCN2C1 ; O=[N+]([O-])c1ccc(N2CCN3CCCC3C2)c(F)c1 ; Nc1ccc(N2CCN3CCCC3C2)c(F)c1 ; CS(=O)c1ncc2cc(C#N)c(=O)n(C3CCCC3)c2n1 ; N#Cc1cc2cnc(Nc3ccc(N4CCN5CCCC5C4)c(F)c3)nc2n(C2CCCC2)c1=O]

Step—1: Synthesis of 2-(2-fluoro-4-nitrophenyl) octahydropyrrolo [1, 2-a]pyrazine: To a stirred solution of 1, 2-difluoro-4-nitrobenzene (500 mg, 3.14 mmol, 1 equiv) in DMF (10 mL), was added K₂CO₃ (1083 mg, 7.85 mmol, 2.5 equiv) and octahydropyrrolo [1, 2-a]pyrazine (436 mg, 3.45 mmol, 1.1 equiv). The resultant reaction mixture was allowed to stir at 100° C. for overnight. Progress of the reaction was monitored by LCMS. After completion of the reaction, diluted with water (100 mL), solid observed was filtered and dried under vacuum to obtain desired product. LCMS: 266 [M+H]⁺

Step—2: Synthesis of 3-fluoro-4-(hexahydropyrrolo [1, 2-a] pyrazin-2(1H)-yl) aniline: To a stirred solution of 2-(2-fluoro-4-nitrophenyl)octahydropyrrolo[1,2-a]pyrazine (400 mg, 1.5 mmol, 1 equiv) in ethanol (6 mL), water (2 mL), was added iron powder (254 mg, 4.5 mmol, 3 equiv) and ammonium chloride (162 mg, 3 mmol, 2 equiv). The resultant reaction mixture was allowed to stir at 90° C. for 1 h. Progress of the reaction was monitored by TLC and LCMS. After completion of the reaction, the mixture was passes through celite bed and the filtrate was concentrated under reduced pressure to obtain desired product. LCMS: 236 [M+H]⁺

Step—3: Synthesis of 8-cyclopentyl-2-((3-fluoro-4-(hexahydropyrrolo[1,2-a]pyrazin-2(1H)-yl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 3-fluoro-4-(hexahydropyrrolo[1,2-a]pyrazin-2(1H)-yl)aniline (85 mg, 0.36 mmol, 1.1 equiv). The resultant reaction mixture was allowed to stir at 100° for 3 h. Progress of the reaction was monitored by LCMS. After completion of the reaction, solid observed was filtered and dried under vacuum to obtain desired product. LCMS: 474 [M+H]⁺; ¹HNMR: ¹H NMR (DMSO-d₆, 400 MHz) δ 10.58 (br. s., 1H), 8.84 (s, 1H), 8.57 (s, 1H), 7.63 (d, J=13.2 Hz, 1H), 7.31 (br. s., 1H), 7.06 (t, J=9.0 Hz, 1H), 5.76 (br. s., 1H), 3.40 (br. s., 1H), 3.25 (br. s., 2H), 3.01 (d, J=10.1 Hz, 2H), 2.70-2.84 (m, 1H), 2.21 (br. s., 3H), 1.97-2.12 (m, 2H), 1.89 (br. s., 1H), 1.79 (br. s., 3H), 1.69 (br. s., 2H), 1.60 (br. s., 2H), 1.26-1.44 (m, 2H).

### Example—S82: Synthesis of 8-cyclopentyl-2-((3-fluoro-4-(2-oxopiperazin-1-yl)phenyl)amino)-7-oxo-7,8-dihydropyrido[2,3-d]pyrimidine-6-carbonitrile (Compound No. 57)

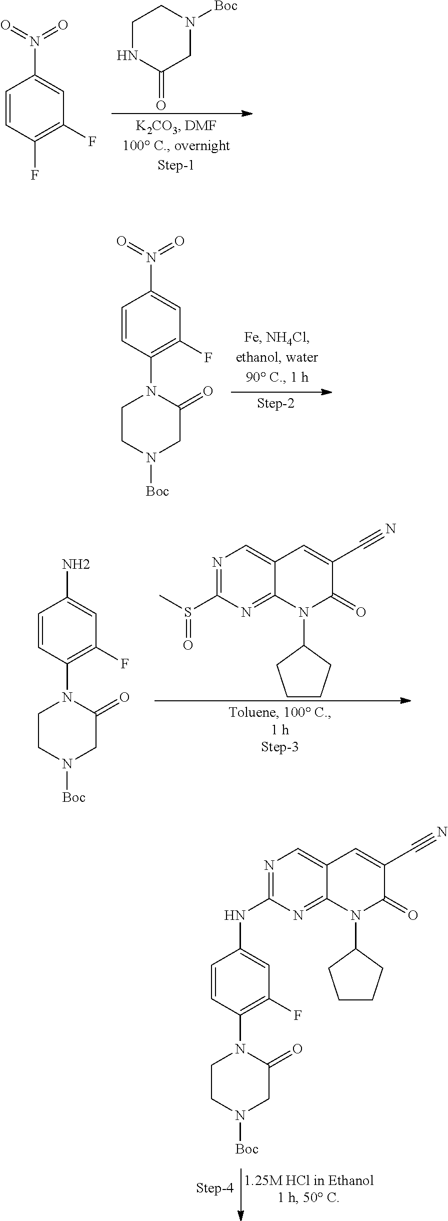 [SMILES: O=[N+]([O-])c1ccc(F)c(F)c1 ; CC(C)(C)OC(=O)N1CCNC(=O)C1 ; CC(C)(C)OC(=O)N1CCN(c2ccc([N+](=O)[O-])cc2F)C(=O)C1 ; CC(C)(C)OC(=O)N1CCN(c2ccc(N)cc2F)C(=O)C1 ; CS(=O)c1ncc2cc(C#N)c(=O)n(C3CCCC3)c2n1 ; CC(C)(C)OC(=O)N1CCN(c2ccc(Nc3ncc4cc(C#N)c(=O)n(C5CCCC5)c4n3)cc2F)C(=O)C1]

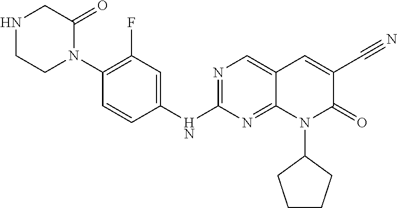 [SMILES: N#Cc1cc2cnc(Nc3ccc(N4CCNCC4=O)c(F)c3)nc2n(C2CCCC2)c1=O]

Step—1: Synthesis of tert-butyl 4-(2-fluoro-4-nitrophenyl)-3-oxopiperazine-1-carboxylate: To a stirred solution of 1, 2-difluoro-4-nitrobenzene (500 mg, 3.14 mmol, 1 equiv) in DMF (10 mL), was added K₂CO₃ (1300 mg, 9.42 mmol, 3 equiv) and tert-butyl 3-oxopiperazine-1-carboxylate (629 mg, 3.14 mmol, 1 equiv). The resultant reaction mixture was allowed to stir at 100° C. for overnight. Progress of the reaction was monitored by LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain desired product. LCMS: 340 [M+H]⁺

Step—2: Synthesis of tert-butyl 4-(4-amino-2-fluorophenyl)-3-oxopiperazine-1-carboxylate: To a stirred solution of tert-butyl 4-(2-fluoro-4-nitrophenyl)-3-oxopiperazine-1-carboxylate (350 mg, 1.03 mmol, 1 equiv) in ethanol (6 mL), water (2 mL), was added iron powder (174 mg, 3.09 mmol, 3 equiv) and ammonium chloride (111 mg, 2.06 mmol, 2 equiv). The resultant reaction mixture was allowed to stir at 90° C. for 1 h. Progress of the reaction was monitored by TLC and LCMS. After completion of the reaction, the mixture was passes through celite bed and the filtrate was concentrated under reduced pressure to obtain crude which was purified by normal phase combi flash to obtain desired product. LCMS: 310 [M+H]⁺

Step—3: Synthesis of tert-butyl 4-(4-((6-cyano-8-cyclopentyl-7-oxo-7,8-dihydropyrido[2,3-d]pyrimidin-2-yl)amino)-2-fluorophenyl)-3-oxopiperazine-1-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added tert-butyl 4-(4-amino-2-fluorophenyl)-3-oxopiperazine-1-carboxylate (113 mg, 0.36 mmol, 1.1 equiv). The resultant reaction mixture was allowed to stir at 100° C. for overnight. Progress of the reaction was monitored by LCMS. After completion of the reaction, solid observed was filtered and dried under vacuum to obtain desired product. LCMS: 548 [M+H]⁺

Step—4: Synthesis of 8-cyclopentyl-2-((3-fluoro-4-(2-oxopiperazin-1-yl)phenyl)amino)-7-oxo-7,8-dihydropyrido[2,3-d]pyrimidine-6-carbonitrile: A solution of tert-butyl 4-(4-((6-cyano-8-cyclopentyl-7-oxo-7,8-dihydropyrido[2,3-d]pyrimidin-2-yl)amino)-2-fluorophenyl)-3-oxopiperazine-1-carboxylate (50 mg, 0.09 mmol, 1 equiv) in 1.25 M HCl in ethanol (5 mL) was allowed to stir at 100° C. for 1 h. Progress of the reaction was monitored by LCMS. After completion of the reaction, solvent was removed under reduced pressure to obtain crude, which was purified by reverse phase HPLC to obtain desired product. LCMS: 448 [M+H]⁺; ¹H NMR (DMSO-d₆, 400 MHz): δ 10.72 (br. s., 1H), 8.90 (s, 1H), 8.62 (s, 1H), 7.80 (d, J=11.8 Hz, 1H), 7.48 (d, J=7.5 Hz, 1H), 7.36 (t, J=8.8 Hz, 1H), 5.83 (br. s., 1H), 3.52 (br. s., 2H), 3.39 (s, 2H), 3.01 (br. s., 2H), 2.22 (br. s., 2H), 1.92 (d, J=14.5 Hz, 2H), 1.82 (br. s., 2H), 1.61 (br. s., 2H).

### Example—S83: Synthesis of 8-cyclopentyl-2-((3-fluoro-4-(octahydro-2H-pyrido[1,2-a]pyrazin-2-yl)phenyl)amino)-7-oxo-7,8-dihydropyrido[2,3-d]pyrimidine-6-carbonitrile (Compound No. 59)

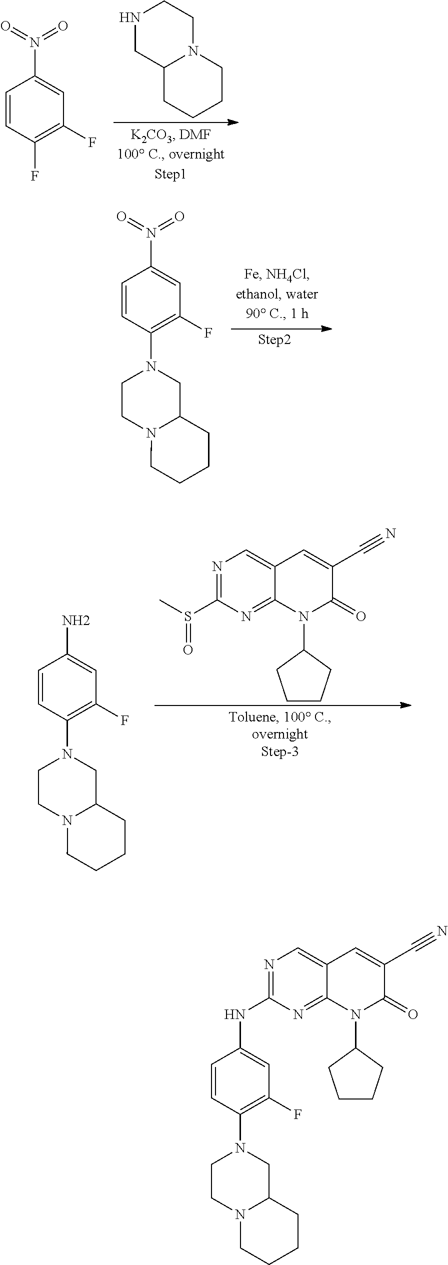 [SMILES: O=[N+]([O-])c1ccc(F)c(F)c1 ; C1CCN2CCNCC2C1 ; O=[N+]([O-])c1ccc(N2CCN3CCCCC3C2)c(F)c1 ; Nc1ccc(N2CCN3CCCCC3C2)c(F)c1 ; CS(=O)c1ncc2cc(C#N)c(=O)n(C3CCCC3)c2n1 ; N#Cc1cc2cnc(Nc3ccc(N4CCN5CCCCC5C4)c(F)c3)nc2n(C2CCCC2)c1=O]

Step—1: Synthesis of 2-(2-fluoro-4-nitrophenyl)octahydro-2H-pyrido[1,2-a]pyrazine: To a stirred solution of 1, 2-difluoro-4-nitrobenzene (500 mg, 3.14 mmol, 1 equiv) in DMF (10 mL), was added K₂CO₃ (1083 mg, 7.85 mmol, 2.5 equiv) and octahydro-2H-pyrido [1, 2-a] pyrazine (484 mg, 3.45 mmol, 1.1 equiv). The resultant reaction mixture was allowed to stir at 100° C. for overnight. Progress of the reaction was monitored by LCMS. After completion of the reaction, the mixture was diluted with ice water (50 mL) and extracted with ethyl acetate (150 mL). Organic layer was washed with water (50 mL) and brine solution (50 mL). Organic layer was dried over anhydrous sodium sulphate and concentrated under reduced pressure to obtain desired product. LCMS: 280 [M+H]⁺

Step—2: Synthesis of 3-fluoro-4-(octahydro-2H-pyrido[1,2-a]pyrazin-2-yl)aniline: To a stirred solution of 2-(2-fluoro-4-nitrophenyl)octahydro-2H-pyrido[1,2-a]pyrazine (500 mg, 1.79 mmol, 1 equiv) in ethanol (6 mL), water (2 mL), was added iron powder (301 mg, 5.37 mmol, 3 equiv) and ammonium chloride (193 mg, 3.58 mmol, 2 equiv). The resultant reaction mixture was allowed to stir at 90° C. for 1 h. Progress of the reaction was monitored by TLC and LCMS. After completion of the reaction, the mixture was passed through celite bed and the filtrate was concentrated under reduced pressure to obtain desired product. LCMS: 250 [M+H]⁺

Step—3: Synthesis of 8-cyclopentyl-2-((3-fluoro-4-(octahydro-2H-pyrido[1,2-a]pyrazin-2-yl)phenyl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 3-fluoro-4-(octahydro-2H-pyrido[1,2-a]pyrazin-2-yl)aniline (90 mg, 0.36 mmol, 1.1 equiv) at RT. The resultant reaction mixture was allowed to stir at 100° C. for overnight. Progress of the reaction was monitored by LCMS. After completion of the reaction, solid observed was filtered and dried under vacuum to obtain desired product. LCMS: 488 [M+H]⁺; ¹HNMR: (DMSO-d₆, 400 MHz): δ 10.57 (br. s., 1H), 8.59 (s, 1H), 7.70 (d, J=14.9 Hz, 1H), 7.37 (br. s., 1H), 7.15 (d, J=9.6 Hz, 1H), 5.79 (br. s., 1H), 3.41 (d, J=10.5 Hz, 4H), 3.08-3.23 (m, 2H), 2.80-3.08 (m, 2H), 2.21 (br. s., 2H), 1.87-1.81 (br. s., 9H), 1.60 (br. s., 4H).

### Example—S84: Synthesis of 8-cyclopentyl-2-((1-(2-(dimethylamino)ethyl)piperidin-4-yl)amino)-7-oxo-7,8-dihydropyrido[2,3-d]pyrimidine-6-carbonitrile (Compound No. 67)

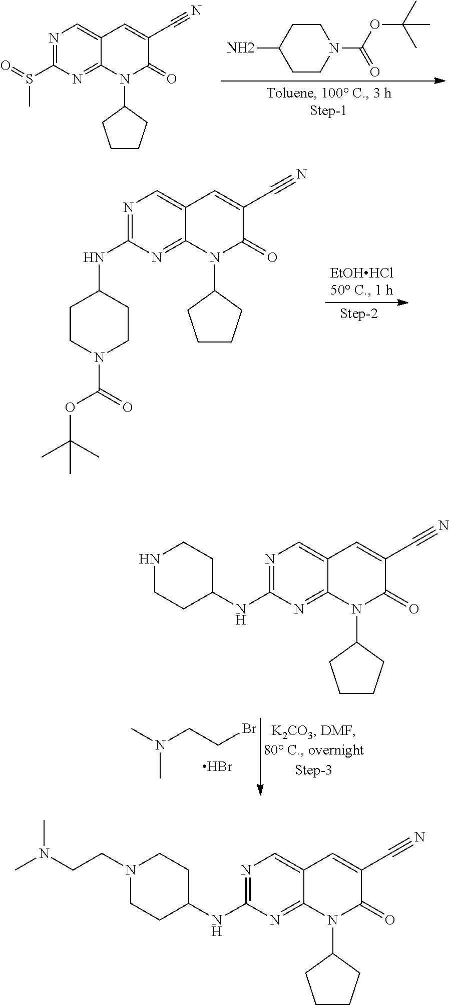

Step—1: Synthesis of tert-butyl 4-((6-cyano-8-cyclopentyl-7-oxo-7,8-dihydropyrido[2,3-d]pyrimidin-2-yl)amino)piperidine-1-carboxylate: To a stirred solution of 8-cyclopentyl-2-(methylsulfinyl)-7-oxo-7, 8-dihydropyrido [2, 3-d] pyrimidine-6-carbonitrile (200 mg, 0.66 mmol, 1 equiv) in toluene (5 mL), was added tert-butyl 4-aminopiperidine-1-carboxylate (144 mg, 0.72 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain crude compound, which was purified by recrystallization with methanol to obtain desired product. LCMS: 439 [M+H]⁺

Step—2: Synthesis of 8-cyclopentyl-7-oxo-2-(piperidin-4-ylamino)-7,8-dihydropyrido[2,3-d]pyrimidine-6-carbonitrile: To a solution of tert-butyl 4-((6-cyano-8-cyclopentyl-7-oxo-7,8-dihydropyrido[2,3-d]pyrimidin-2-yl)amino)piperidine-1-carboxylate (200 mg, 0.45 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain desired product. LCMS: 339 [M+H]⁺

Step—3: Synthesis of 8-cyclopentyl-2-((1-(2-(dimethylamino)ethyl)piperidin-4-yl)amino)-7-oxo-7,8-dihydropyrido[2,3-d]pyrimidine-6-carbonitrile: To a stirred solution of 8-cyclopentyl-7-oxo-2-(piperidin-4-ylamino)-7,8-dihydropyrido[2,3-d]pyrimidine-6-carbonitrile (60 mg, 0.16 mmol, 1 equiv) in DMF (5 mL), was added K₂CO₃ (44 mg, 0.32 mmol, 2 equiv) and 2-bromo-N,N-dimethylethan-1-amine.HBr (66 mg, 0.32 mmol, 2 equiv). The resultant reaction mixture was allowed to stir at 80° C. for overnight. Progress of the reaction was monitored by LCMS. After completion of the reaction, the reaction mixture was diluted with water (25 mL) and extracted with ethyl acetate (50 mL×2). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 410 [M+H]⁺; ¹H NMR (400 MHz, DMSO-d₆): δ 8.66 (s, 1H), 8.37-8.52 (m, 1H), 8.28 (d, J=7.3 Hz, 1H), 5.86 (br. s., 1H), 5.58-5.76 (m, 1H), 3.88 (br. s., 1H), 3.74 (br. s., 1H), 2.91 (d, J=12.2 Hz, 2H), 2.19-2.42 (m, 5H), 2.12 (s, 6H), 1.91-2.02 (m, 4H), 1.75-1.90 (m, 4H), 1.66 (br. s., 1H), 1.55 (d, J=11.2 Hz, 2H).

### Example—S85: Synthesis of 1-cyclopentyl-7-((4-(4-methylpiperazin-1-yl) phenyl) amino)-2-oxo-1, 2-dihydro-1, 6-naphthyridine-3-carbonitrile (Compound No. 87)

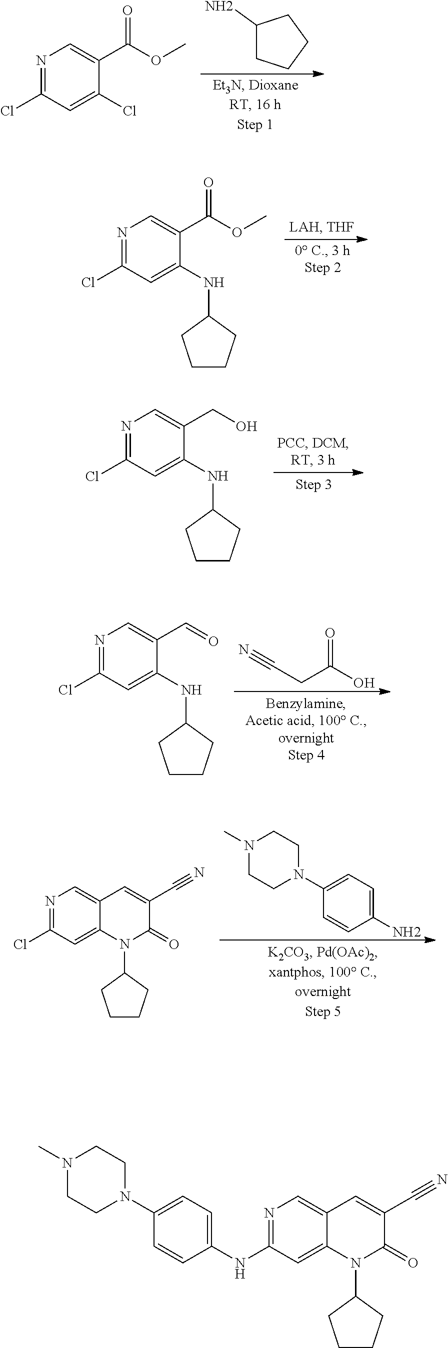

Step—1: Synthesis of methyl 6-chloro-4-(cyclopentylamino)nicotinate: To a stirred solution of methyl 4,6-dichloronicotinate (3500 mg, 15.9 mmol, 1 equiv) in Dioxane (40 mL), was added ET₃N (7 mL, 48 mmol, 3 equiv) and cyclopentanamine (1623 mg, 19 mmol, 1.2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain desired product. LCMS: 255 [M+H]⁺

Step—2: Synthesis of (6-chloro-4-(cyclopentylamino)pyridin-3-yl)methanol: To a stirred solution of methyl 6-chloro-4-(cyclopentylamino) nicotinate (3500 mg, 13.77 mmol, 1 equiv) in THF (40 mL), was added LAH (1047 mg, 27.55 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium sulphate (100 mL) at 0° C. and then passes through celite bed, filtrate obtain was diluted with ethyl acetate (150 mL) and washed with water (100 mL), brine solution (100 mL). Organic layer was dried over anhydrous sodium sulphate and concentrated under reduced pressure to obtain desired product. LCMS: 227 [M+H]⁺

Step—3: Synthesis of 6-chloro-4-(cyclopentylamino)nicotinaldehyde: To a stirred solution of (6-chloro-4-(cyclopentylamino)pyridin-3-yl)methanol (3000 mg, 13.2 mmol, 1 equiv) in DCM (30 mL), was added PCC (2867 mg, 13.2 mmol, 1 equiv) at RT. The reaction mixture was allowed to stir at RT for 3 h. Progress of the reaction was monitored by TLC and LCMS. After completion of the reaction, the reaction mixture was passes through celite bed; filtrate was diluted with DCM (150 mL) and washed with water (100 mL), brine solution (100 mL). Organic layer was dried over anhydrous sodium sulphate and concentrated under reduced pressure to obtain desired product. LCMS: 225 [M+H]⁺

Step—4: Synthesis of 7-chloro-1-cyclopentyl-2-oxo-1,2-dihydro-1,6-naphthyridine-3-carbonitrile: To a stirred solution of 6-chloro-4-(cyclopentylamino)nicotinaldehyde (2500 mg, 11.16 mmol, 1 equiv) in acetic acid (30 mL), was added Cyanoacetic acid (1138 mg, 13.9 mmol, 1.2 equiv) and benzyl amine (0.1 mL, 1.11 mmol, 0.1 equiv). The reaction mixture was allowed to stir at 100° C. for overnight. Progress of the reaction was monitored by TLC and LCMS. After completion of the reaction, the reaction mixture was diluted with water (30 mL), solid observed was filtered and dried under vacuum to obtain desired product. LCMS: 274 [M+H]⁺

Step—5: Synthesis of 1-cyclopentyl-7-((4-(4-methylpiperazin-1-yl)phenyl)amino)-2-oxo-1,2-dihydro-1,6-naphthyridine-3-carbonitrile: To a solution of 7-chloro-1-cyclopentyl-2-oxo-1,2-dihydro-1,6-naphthyridine-3-carbonitrile (100 mg, 0.36 mmol, 1 equiv) in dioxane (5 mL), was added 4-(4-methylpiperazin-1-yl)aniline (77 mg, 0.4 mmol, 1.1 equiv) and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 429 [M+H]⁺, ¹H NMR (400 MHz, DMSO-d₆): δ 9.53 (s, 1H), 8.54 (d, J=8.8 Hz, 2H), 7.37 (d, J=9.2 Hz, 2H), 6.96 (d, J=9.2 Hz, 2H), 6.47 (s, 1H), 5.38 (br. s., 1H), 3.10 (d, J=4.8 Hz, 4H), 2.24 (s, 3H), 2.02 (br. s., 4H), 1.83 (br. s., 4H), 1.60 (br. s., 4H).

### Example—S86: Synthesis of 1-cyclopentyl-2-oxo-7-((4-(piperazin-4-yl) phenyl) amino)-1, 2-dihydro-1, 6-naphthyridine-3-carbonitrile (Compound No. 88)

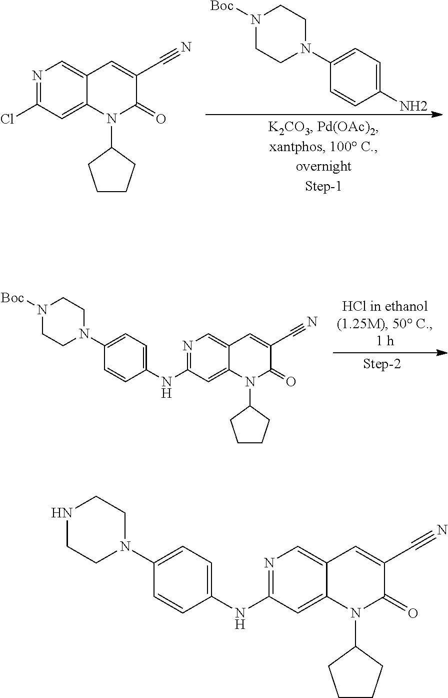 [SMILES: N#Cc1cc2cnc(Cl)cc2n(C2CCCC2)c1=O ; CC(C)(C)OC(=O)N1CCN(c2ccc(N)cc2)CC1 ; CC(C)(C)OC(=O)N1CCN(c2ccc(Nc3cc4c(cn3)cc(C#N)c(=O)n4C3CCCC3)cc2)CC1 ; N#Cc1cc2cnc(Nc3ccc(N4CCNCC4)cc3)cc2n(C2CCCC2)c1=O]

Step—1: Synthesis of tert-butyl 4-(4-((3-cyano-1-cyclopentyl-2-oxo-1,2-dihydro-1,6-naphthyridin-7-yl)amino)phenyl)piperazin-1-carboxylate: To a solution of 7-chloro-1-cyclopentyl-2-oxo-1,2-dihydro-1,6-naphthyridine-3-carbonitrile (100 mg, 0.36 mmol, 1 equiv) in dioxane (5 mL), was added tert-butyl 4-(4-aminophenyl)piperazine-1-carboxylate (111 mg, 0.4 mmol, 1.1 equiv) and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain desired product. LCMS: 515 [M+H]⁺

Step—2: Synthesis of 1-cyclopentyl-2-oxo-7-((4-(piperazin4-yl)phenyl)amino)-1,2-dihydro-1,6-naphthyridine-3-carbonitrile: A solution of tert-butyl 4-(4-((3-cyano-1-cyclopentyl-2-oxo-1,2-dihydro-1,6-naphthyridin-7-yl)amino)phenyl)piperazin-1-carboxylate (150 mg, 0.29 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain crude, which was purified by reverse phase HPLC to obtain desired product. LCMS: 415 [M+H]⁺, ¹H NMR (400 MHz, DMSO-d₆): δ 9.54 (s, 1H), 8.54 (d, J=8.8 Hz, 2H), 8.23 (s, 1H), 7.38 (d, J=8.8 Hz, 2H), 6.96 (d, J=9.2 Hz, 2H), 6.48 (s, 1H), 5.37 (br. s., 1H), 3.01-3.12 (m, 4H), 2.83-3.01 (m, 4H), 2.02 (br. s., 2H), 1.83 (br. s., 4H), 1.61 (br. s., 2H).

### Example—S87: Synthesis of 1-cyclopentyl-7-((4-(1-methylpiperidin-4-yl) phenyl) amino)-2-oxo-1, 2-dihydro-1, 6-naphthyridine-3-carbonitrile (Compound No. 89)

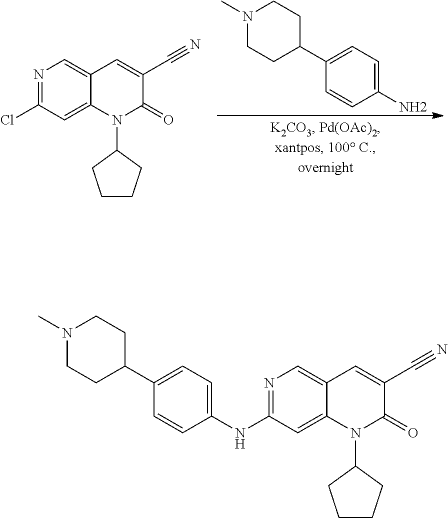 [SMILES: N#Cc1cc2cnc(Cl)cc2n(C2CCCC2)c1=O ; CN1CCC(c2ccc(N)cc2)CC1 ; CN1CCC(c2ccc(Nc3cc4c(cn3)cc(C#N)c(=O)n4C3CCCC3)cc2)CC1]

To a solution of 7-chloro-1-cyclopentyl-2-oxo-1,2-dihydro-1,6-naphthyridine-3-carbonitrile (100 mg, 0.36 mmol, 1 equiv) in dioxane (5 mL), was added 4-(1-methylpiperidin-4-yl)aniline (77 mg, 0.4 mmol, 1.1 equiv) and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 429 [M+H]⁺, ¹H NMR (400 MHz, DMSO-d₆): δ 9.68 (s, 1H), 8.58 (d, J=7.9 Hz, 2H), 7.49 (d, J=8.3 Hz, 2H), 7.23 (d, J=8.3 Hz, 2H), 6.61 (s, 1H), 5.34 (br. s., 1H), 2.90 (d, J=9.6 Hz, 2H), 2.23 (s, 3H), 1.93-2.14 (m, 4H), 1.87 (br. s., 4H), 1.69-1.75 (m, 3H), 1.65 (d, J=12.3 Hz, 4H).

### Example—S88: Synthesis of 1-cyclopentyl-2-oxo-7-((1,2,3,4-tetrahydroisoquinolin-6-yl)amino)-1,2-dihydro-1,6-naphthyridine-3-carbonitrile (Compound No. 90)

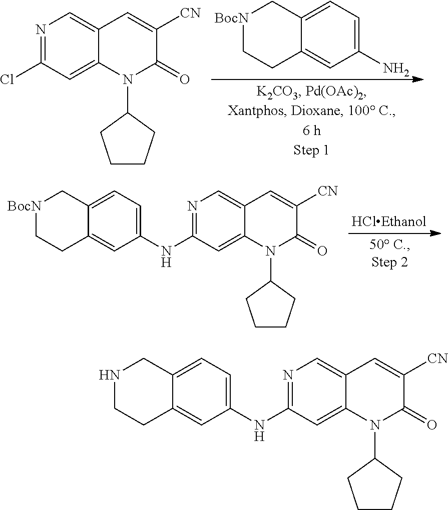

Step 1: Synthesis of tert-butyl 6-((3-cyano-1-cyclopentyl-2-oxo-1,2-dihydro-1,6-naphthyridin-7-yl)amino)-3,4-dihydroisoquinoline-2(1H)-carboxylate: To a solution of 7-chloro-1-cyclopentyl-2-oxo-1,2-dihydro-1,6-naphthyridine-3-carbonitrile (100 mg, 0.37 mmol, 1 equiv) in dioxane (5 mL), was added tert-butyl 6-amino-3,4-dihydroisoquinoline-2(1H)-carboxylate (100 mg, 0.39 mmol, 1.1 equiv) and potassium carbonate (150 mg, 1.1 mmol, 3 equiv). The reaction mixture was purged with nitrogen gas for 10 min., followed by the addition of palladium acetate (8 mg, 0.037 mmol, 0.1 equiv) and xantphos (42 mg, 0.073 mmol, 0.2 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain product. LCMS: 486 [M+H]⁺

Step 2: Synthesis of 1-cyclopentyl-2-oxo-7-((1,2,3,4-tetrahydroisoquinolin-6-yl)amino)-1,2-dihydro-1,6-naphthyridine-3-carbonitrile: A solution of tert-butyl 6-((3-cyano-1-cyclopentyl-2-oxo-1,2-dihydro-1,6-naphthyridin-7-yl)amino)-3,4-dihydroisoquinoline-2(1H)-carboxylate (250 mg, 0.52 mmol, 1 equiv) in 1.25 M HCl in ethanol (8 mL) was allowed to stir for 1 h at 50° C. Progress of the reaction was monitored by LCMS. After completion of the reaction, solvent was removed under reduced pressure to obtain residue, which was dried under lyophilizer to obtain the crude product which was purified by reverse phase. LCMS: 386 [M+H]⁺; ¹H NMR (METHANOL-d₄, 400 MHz): δ 8.56 (s, 1H), 8.40 (s, 1H), 7.52 (s, 1H), 7.40-7.46 (m, 2H), 7.18 (d, J=8.8 Hz, 1H), 6.73 (s, 1H), 5.35-5.44 (m, 1H), 4.26 (s, 2H), 3.43 (t, J=6.4 Hz, 2H), 3.07 (t, J=6.6 Hz, 2H), 2.16-2.27 (m, 2H), 2.01 (dd, J=12.7, 9.2 Hz, 4H), 1.73 (d, J=4.8 Hz, 2H).

### Example—S89: Synthesis of 8-cyclopentyl-2-((1-(methylsulfonyl)piperidin-4-yl)amino)-7-oxo-7,8-dihydropyrido[2,3-d]pyrimidine-6-carbonitrile (Compound No. 93)

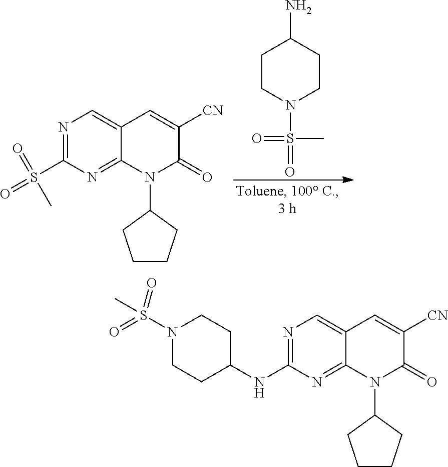

To a stirred solution of 8-cyclopentyl-2-(methylsulfonyl)-7-oxo-7,8-dihydropyrido[2,3-d]pyrimidine-6-carbonitrile (100 mg, 0.33 mmol, 1 equiv) in toluene (5 mL), was added 1-(methylsulfonyl)piperidin-4-amine (64 mg, 0.36 mmol, 1.1 equiv). The resultant reaction mixture was allowed to stir at 100° for 3 h. Progress of the reaction was monitored by LCMS. After completion of the reaction, solvent was removed under reduced pressure to obtain crude, which was purified by recrystallization with methanol to obtain desired product. LCMS: 417 [M+H]⁺, ¹H NMR (400 MHz, DMSO-d₆): δ 8.69 (br. s., 1H), 8.43 (s, 1H), 8.14 (br. s., 1H), 5.80 (br. s., 1H), 3.97 (br. s., 1H), 3.61 (d, J=11.8 Hz, 2H), 2.73-2.98 (m, 5H), 2.30 (d, J=11.0 Hz, 2H), 1.98 (br. s., 4H), 1.80 (br. s., 2H), 1.67 (d, J=11.0 Hz, 4H).

### Example—S90: Synthesis of 1-cyclopentyl-2-oxo-7-((4-(piperazin-1-yl) phenyl) amino)-1, 2-dihydro-1, 8-naphthyridine-3-carbonitrile (Compound No. 94)

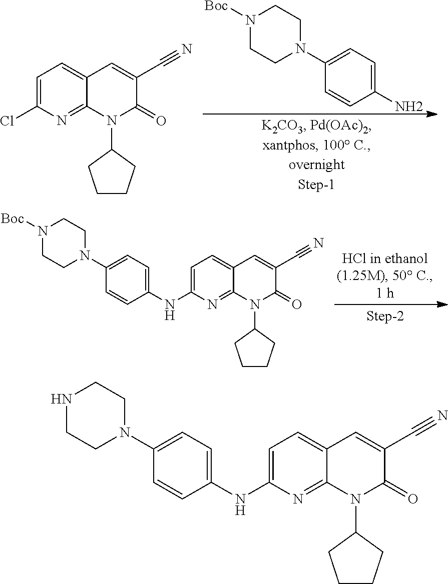

Step—1: Synthesis of tert-butyl 4-(4-((6-cyano-8-cyclopentyl-7-oxo-7,8-dihydro-1,8-naphthyridin-2-yl)amino)phenyl)piperazine-1-carboxylate: To a solution of 7-chloro-1-cyclopentyl-2-oxo-1,2-dihydro-1,8-naphthyridine-3-carbonitrile (100 mg, 0.36 mmol, 1 equiv) in dioxane (5 mL), was added tert-butyl 4-(4-aminophenyl)piperazine-1-carboxylate (111 mg, 0.4 mmol, 1.1 equiv) and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain desired product. LCMS: 515 [M+H]⁺

Step—2: Synthesis of 1-cyclopentyl-2-oxo-7-((4-(piperazin-1-yl)phenyl)amino)-1,2-dihydro-1,8-naphthyridine-3-carbonitrile: A solution of tert-butyl 4-(4-((6-cyano-8-cyclopentyl-7-oxo-7,8-dihydro-1,8-naphthyridin-2-yl)amino)phenyl)piperazine-1-carboxylate (mg, mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain crude, which was purified by reverse phase HPLC to obtain desired product. LCMS: 415 [M+H]⁺, ¹H NMR (400 MHz, DMSO-d₆): δ 9.91 (br. s., 1H), 8.41 (s, 1H), 7.81 (d, J=8.8 Hz, 1H), 7.27-7.54 (m, J=8.3 Hz, 2H), 6.83-7.05 (m, J=8.8 Hz, 2H), 6.70 (d, J=8.8 Hz, 1H), 5.93 (br. s., 1H), 3.08 (br. s., 4H), 2.90 (br. s., 4H), 2.23 (br. s., 2H), 1.86 (br. s., 2H), 1.73 (br. s., 2H), 1.57 (br. s., 2H).

### Example—S91: Synthesis of 1-cyclopentyl-2-oxo-7-((1,2,3,4-tetrahydroisoquinolin-7-yl)amino)-1,2-dihydro-1,6-naphthyridine-3-carbonitrile (Compound No. 95)

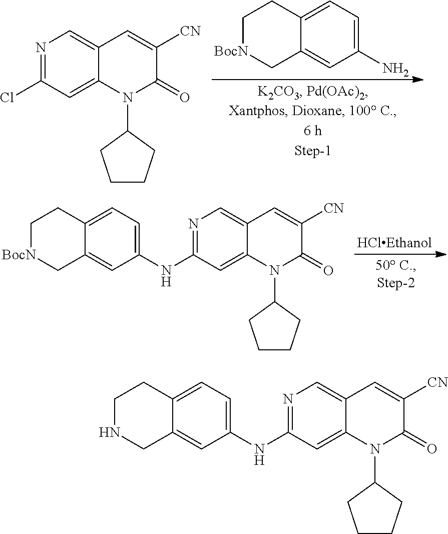

Step 1: Synthesis of tert-butyl 7-((3-cyano-1-cyclopentyl-2-oxo-1,2-dihydro-1,6-naphthyridin-7-yl)amino)-3,4-dihydroisoquinoline-2(1H)-carboxylate: To a solution of 7-chloro-1-cyclopentyl-2-oxo-1,2-dihydro-1,6-naphthyridine-3-carbonitrile (100 mg, 0.37 mmol, 1 equiv) in dioxane (5 mL), was added tert-butyl 7-amino-3,4-dihydroisoquinoline-2(1H)-carboxylate (100 mg, 0.39 mmol, 1.1 equiv) and potassium carbonate (150 mg, 1.1 mmol, 3 equiv). The reaction mixture was purged with nitrogen gas for 10 min., followed by the addition of palladium acetate (8 mg, 0.037 mmol, 0.1 equiv) and xantphos (42 mg, 0.036 mmol, 0.2 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain product. LCMS: 486 [M+H]⁺

Step 2: Synthesis of 1-cyclopentyl-2-oxo-7-((1,2,3,4-tetrahydroisoquinolin-7-yl)amino)-1,2-dihydro-1,6-naphthyridine-3-carbonitrile: A solution of tert-butyl 7-((3-cyano-1-cyclopentyl-2-oxo-1,2-dihydro-1,6-naphthyridin-7-yl)amino)-3,4-dihydroisoquinoline-2(1H)-carboxylate (250 mg, 0.52 mmol, 1 equiv) in 1.25 M HCl in ethanol (8 mL) was allowed to stir for 1 h at 50° C. Progress of the reaction was monitored by LCMS. After completion of the reaction, solvent was removed under reduced pressure to obtain residue, which was dried under lyophilizer to obtain crude product which was purified by reverse phase HPLC. LCMS: 386 [M+H]⁺; ¹H NMR (400 MHz, DMSO-d₆): δ 9.76 (s, 1H), 8.59 (s, 2H), 8.57 (s, 1H), 8.34 (br. s., 2H), 7.36 (br. s., 1H), 7.10 (d, J=8.8 Hz, 1H), 6.68 (s, 1H), 5.25-5.41 (m, 2H), 3.99 (br. s., 2H), 3.10 (br. s., 2H), 2.77 (br. s., 2H), 2.02-2.15 (m, 2H), 1.91 (dd, J=13.6, 7.9 Hz, 4H), 1.64 (br. s., 2H)

### Example—S92: Synthesis of 1-cyclopentyl-7-((4-(4-methylpiperazin-1-yl)phenyl)amino)-2-oxo-1,2-dihydro-1,8-naphthyridine-3-carbonitrile (Compound No. 96)

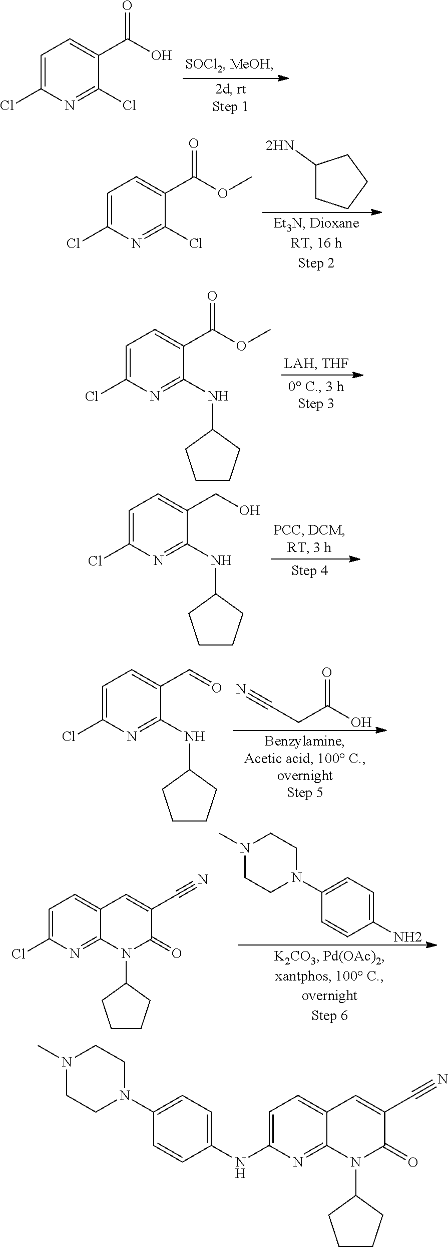

Step 1: Synthesis of Methyl 2,6-dichloronicotinate: To the stirred solution of 2,6-dichloronicotinic acid (6.0 g, 31.2 mmol, 1.0 eq) in 50 mL MeOH was added SOCl₂ (18.6 g, 156.2 mmol, 5.0 equiv) at 0° C. and stirred for 1.5 h at same temperature. The reaction mixture was then stirred at RT for 2 days. The solvent was removed after the completion of the reaction (monitored by LCMS) to give the off white residue. The residue was dissolved in EtOAc (200 mL) and successively washed with aq. NaHCO₃(100 mL), water (100 mL) and brine (50 mL). Organic layer was separated and dried over sodium sulfate. Removal of the solvent under reduced pressure gives the pure product. LCMS: 206 [M+H]⁺

Step—2: Synthesis of methyl 6-chloro-2-(cyclopentylamino)nicotinate: To a stirred solution of methyl 2,6-dichloronicotinate (7000 mg, 34.14 mmol, 1 equiv) in Dioxane (40 mL), was added ET₃N (14 mL, 102.4 mmol, 3 equiv) and cyclopentanamine (3482 mg, 41 mmol, 1.2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain methyl 6-chloro-4-(cyclopentylamino) nicotinate (7000 mg, 80%) as a transparent oil compound. LCMS: 255 [M+H]⁺

Step—3: Synthesis of (6-chloro-2-(cyclopentylamino) pyridin-3-yl) methanol: To a stirred solution of methyl 6-chloro-2-(cyclopentylamino) nicotinate (7000 mg, 27.55 mmol, 1 equiv) in THF (70 mL), was added LAH (2094 mg, 55.11 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium sulphate (100 mL) at 0° C. and then passes through celite bed, filtrate obtain was diluted with ethyl acetate (150 mL) and washed with water (100 mL), brine solution (100 mL). Organic layer was dried over anhydrous sodium sulphate and concentrated under reduced pressure to obtain (6-chloro-4-(cyclopentylamino) pyridin-3-yl) methanol (6000 mg, 96%) as a brown color viscous compound. LCMS: 227 [M+H]⁺

Step—4: Synthesis of 6-chloro-2-(cyclopentylamino)nicotinaldehyde: To a stirred solution of (6-chloro-2-(cyclopentylamino)pyridin-3-yl)methanol (6000 mg, 26.5 mmol, 1 equiv) in DCM (60 mL), was added PCC (5735 mg, 26.5 mmol, 1 equiv) at RT. The reaction mixture was allowed to stir at RT for 3 h. Progress of the reaction was monitored by TLC and LCMS. After completion of the reaction, the reaction mixture was passes through celite bed; filtrate was diluted with DCM (150 mL) and washed with water (100 mL), brine solution (100 mL). Organic layer was dried over anhydrous sodium sulphate and concentrated under reduced pressure to obtain 6-chloro-4-(cyclopentylamino) nicotinaldehyde (6000 mg, quantitative yield) as a dark brown solid compound. LCMS: 225 [M+H]⁺

Step—5: Synthesis of 7-chloro-1-cyclopentyl-2-oxo-1,2-dihydro-1,8-naphthyridine-3-carbonitrile: To a stirred solution of 6-chloro-4-(cyclopentylamino)nicotinaldehyde (6000 mg, 26.78 mmol, 1 equiv) in Acetic acid (50 mL), was added Cyanoacetic acid (2732 mg, 32.1 mmol, 1.2 equiv) and Benzyl amine (0.3 mL, 2.67 mmol, 0.1 equiv). The reaction mixture was allowed to stir at 100° C. for overnight. Progress of the reaction was monitored by TLC and LCMS. After completion of the reaction, the reaction mixture was diluted with water (30 mL), solid observed was filtered and dried under vacuum to obtain 7-chloro-1-cyclopentyl-2-oxo-1,2-dihydro-1,6-naphthyridine-3-carbonitrile (2500 mg, 34%) as a dark brown color solid compound. LCMS: 274 [M+H]⁺

Step—6: Synthesis of 1-cyclopentyl-7-((4-(4-methylpiperazin-1-yl)phenyl)amino)-2-oxo-1,2-dihydro-1,8-naphthyridine-3-carbonitrile: To a solution of 7-chloro-1-cyclopentyl-2-oxo-1,2-dihydro-1,8-naphthyridine-3-carbonitrile (100 mg, 0.36 mmol, 1 equiv) in dioxane (5 mL), was added 4-(4-methylpiperazin-1-yl)aniline (77 mg, 0.4 mmol, 1.1 equiv) and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain product. LCMS: 429 [M+H]⁺; ¹HNMR (400 MHz, DMSO-d₆): δ 9.90 (br. s., 1H), 8.41 (s, 1H), 8.21 (br. s., 1H), 7.81 (d, J=8.3 Hz, 1H), 7.44 (d, J=7.9 Hz, 2H), 6.96 (d, J=8.8 Hz, 2H), 6.69 (d, J=8.8 Hz, 1H), 5.88-5.98 (m, 1H), 3.05-3.16 (m, 4H), 2.22 (s, 4H), 1.86 (br. s., 2H), 1.74 (d, J=5.7 Hz, 2H), 1.51-1.62 (m, 2H)

### Example—S93: Synthesis of 1-cyclopentyl-2-oxo-7-((1,2,3,4-tetrahydroisoquinolin-6-yl)amino)-1,2-dihydro-1,8-naphthyridine-3-carbonitrile (Compound No. 97)

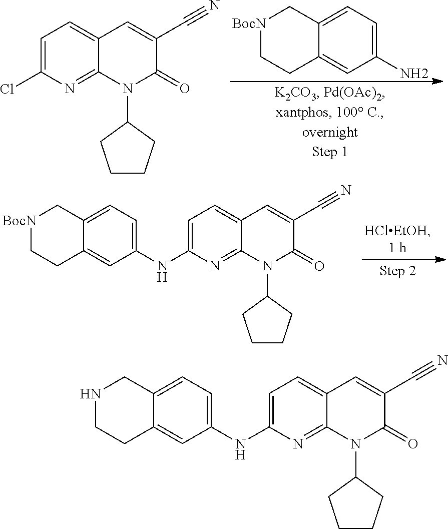

Step—1: Synthesis of tert-butyl 6-((6-cyano-8-cyclopentyl-7-oxo-7,8-dihydro-1,8-naphthyridin-2-yl)amino)-3,4-dihydroisoquinoline-2(1H)-carboxylate: To a solution of 7-chloro-1-cyclopentyl-2-oxo-1,2-dihydro-1,8-naphthyridine-3-carbonitrile (100 mg, 0.36 mmol, 1 equiv) in dioxane (5 mL), was added tert-butyl 6-amino-3,4-dihydroisoquinoline-2(1H)-carboxylate (99 mg, 0.4 mmol, 1.1 equiv and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude. LCMS: 486 [M+H]⁺

Step—2: Synthesis of 1-cyclopentyl-2-oxo-7-((1,2,3,4-tetrahydroisoquinolin-6-yl)amino)-1,2-dihydro-1,8-naphthyridine-3-carbonitrile: A solution of tert-butyl 6-((6-cyano-8-cyclopentyl-7-oxo-7,8-dihydro-1,8-naphthyridin-2-yl)amino)-3,4-dihydroisoquinoline-2(1H)-carboxylate (30 mg, 0.063 mmol, 1 equiv) in 1.25 M HCl in ethanol (5 mL) was allowed to stir for 1 h at 50° C. Progress of the reaction was monitored by LCMS. After completion of the reaction, reaction mixture was cooled to room temperature which results in the formation of precipitates which were filtered and washed with ethanol to obtain the crude product which was purified by reverse phase. LCMS: 386 [M+H]⁺; ¹H NMR (DMSO-d₆, 400 MHz): δ 10.10 (br. s., 1H), 8.46 (s, 1H), 8.29 (br. s., 1H), 7.88 (d, J=8.8 Hz, 1H), 7.64 (br. s., 1H), 7.26 (d, J=8.3 Hz, 1H), 7.07 (d, J=8.3 Hz, 1H), 6.77 (d, J=8.3 Hz, 1H), 5.91-6.09 (m, 1H), 3.95 (br. s., 2H), 3.08 (br. s., 2H), 2.78 (br. s., 2H), 2.23 (br. s., 2H), 1.95 (br. s., 2H), 1.79 (br. s., 2H), 1.60 (d, J=4.8 Hz, 2H)

### Example—S94: Synthesis of 1-cyclopentyl-7-((4-(1-methylpiperidin-4-yl) phenyl) amino)-2-oxo-1, 2-dihydro-1, 8-naphthyridine-3-carbonitrile (Compound No. 98)

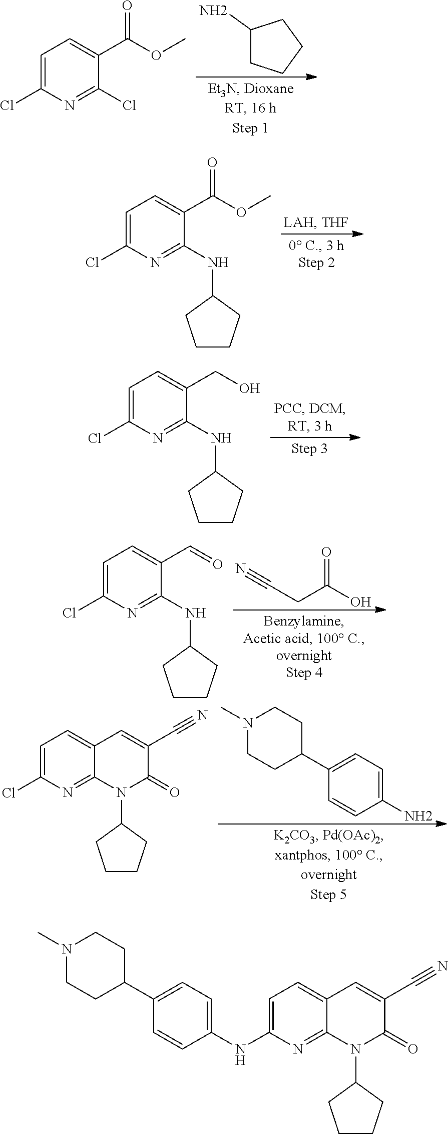

Step—1: Synthesis of methyl 6-chloro-4-(cyclopentylamino)nicotinate: To a stirred solution of methyl 4,6-dichloronicotinate (7000 mg, 34.14 mmol, 1 equiv) in Dioxane (40 mL), was added ET₃N (14 mL, 102.4 mmol, 3 equiv) and cyclopentanamine (3482 mg, 41 mmol, 1.2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain desired product. LCMS: 255 [M+H]⁺

Step—2: Synthesis of (6-chloro-4-(cyclopentylamino) pyridin-3-yl) methanol: To a stirred solution of methyl 6-chloro-4-(cyclopentylamino) nicotinate (7000 mg, 27.55 mmol, 1 equiv) in THF (70 mL), was added LAH (2094 mg, 55.11 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium sulphate (100 mL) at 0° C. and then passes through celite bed, filtrate obtain was diluted with ethyl acetate (150 mL) and washed with water (100 mL), brine solution (100 mL). Organic layer was dried over anhydrous sodium sulphate and concentrated under reduced pressure to obtain desired product. LCMS: 227 [M+H]⁺

Step—3: Synthesis of 6-chloro-4-(cyclopentylamino)nicotinaldehyde: To a stirred solution of (6-chloro-4-(cyclopentylamino)pyridin-3-yl)methanol (6000 mg, 26.5 mmol, 1 equiv) in DCM (60 mL), was added PCC (5735 mg, 26.5 mmol, 1 equiv) at RT. The reaction mixture was allowed to stir at RT for 3 h. Progress of the reaction was monitored by TLC and LCMS. After completion of the reaction, the reaction mixture was passes through celite bed; filtrate was diluted with DCM (150 mL) and washed with water (100 mL), brine solution (100 mL). Organic layer was dried over anhydrous sodium sulphate and concentrated under reduced pressure to obtain desired product. LCMS: 225 [M+H]⁺

Step—4: Synthesis of 7-chloro-1-cyclopentyl-2-oxo-1,2-dihydro-1,6-naphthyridine-3-carbonitrile: To a stirred solution of 6-chloro-4-(cyclopentylamino)nicotinaldehyde (6000 mg, 26.78 mmol, 1 equiv) in Acetic acid (50 mL), was added Cyanoacetic acid (2732 mg, 32.1 mmol, 1.2 equiv) and Benzyl amine (0.3 mL, 2.67 mmol, 0.1 equiv). The reaction mixture was allowed to stir at 100° C. for overnight. Progress of the reaction was monitored by TLC and LCMS. After completion of the reaction, the reaction mixture was diluted with water (30 mL), solid observed was filtered and dried under vacuum to obtain desired product. LCMS: 274 [M+H]⁺

Step—5: Synthesis of 1-cyclopentyl-7-((4-(1-methylpiperidin-4-yl)phenyl)amino)-2-oxo-1,2-dihydro-1,8-naphthyridine-3-carbonitrile: To a solution of 7-chloro-1-cyclopentyl-2-oxo-1,2-dihydro-1,6-naphthyridine-3-carbonitrile (100 mg, 0.36 mmol, 1 equiv) in dioxane (5 mL), was added 4-(1-methylpiperidin-4-yl)aniline (77 mg, 0.4 mmol, 1.1 equiv) and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 428 [M+H]⁺, ¹H NMR (400 MHz, DMSO-d₆): δ 10.05 (s, 1H), 8.45 (s, 1H), 7.87 (d, J=8.8 Hz, 1H), 7.38-7.58 (m, J=8.3 Hz, 2H), 7.11-7.33 (m, J=8.3 Hz, 2H), 6.76 (d, J=8.8 Hz, 1H), 5.93 (s, 1H), 2.90 (d, J=11.4 Hz, 2H), 2.44 (m, 1H), 2.23 (s, 5H), 2.03 (t, J=10.3 Hz, 2H), 1.85 (br. s., 2H), 1.61-1.78 (m, 6H), 1.57 (d, J=5.3 Hz, 2H).

### Example—S95: Synthesis of 8-cyclobutyl-7-oxo-2-((1,2,3,4-tetrahydroisoquinolin-6-yl)amino)-7,8-dihydropyrido[2,3-d]pyrimidine-6-carbonitrile hydrochloride (Compound No. 91)

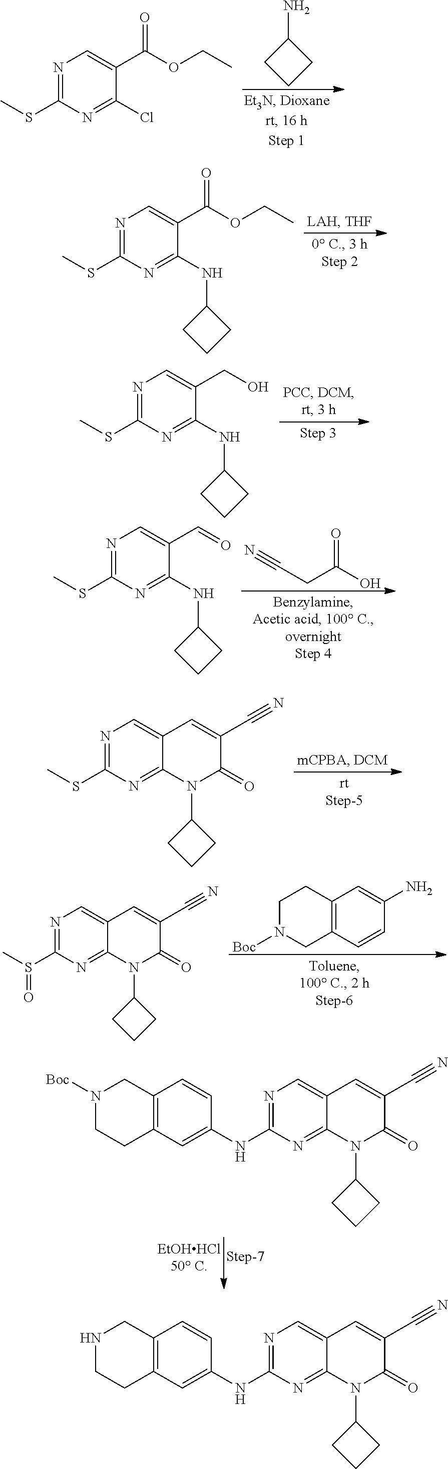

Step—1: Synthesis of ethyl 4-(cyclobutylamino)-2-(methylthio)pyrimidine-5-carboxylate: To a stirred solution of ethyl 4-chloro-2-(methylthio)pyrimidine-5-carboxylate (2000 mg, 8.58 mmol, 1 equiv) in Dioxane (20 mL), was added Et₃N (3.6 mL, 10.3 mmol, 1.2 equiv) and cyclobutanamine (752 mg, 10.3 mmol, 1.2 equiv) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain the product. LCMS: 268 [M+H]⁺

Step—2: Synthesis of (4-(cyclobutylamino)-2-(methylthio)pyrimidin-5-yl)methanol: To a stirred solution of ethyl 4-(cyclobutylamino)-2-(methylthio)pyrimidine-5-carboxylate (2000 mg, 7.4 mmol, 1 equiv) in THF (50 mL), was added LAH (565 mg, 14.8 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium hydroxide (100 mL) at 0° C. and extracted with ethyl acetate (150 mL×2). Organic layer was washed with water (100 mL) and brine solution (100 mL). Organic layer was dried over anhydrous sodium sulphate and concentrated under reduced pressure to obtain the product. LCMS: 226 [M+H]⁺

Step—3: Synthesis of 4-(cyclobutylamino)-2-(methylthio)pyrimidine-5-carbaldehyde: To a stirred solution of (4-(cyclobutylamino)-2-(methylthio)pyrimidin-5-yl)methanol (1400 mg, 6.16 mmol, 1 equiv) in DCM (30 mL), was added PCC (1332 mg, 6.16 mmol, 1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain the product. LCMS: 224 [M+H]⁺

Step—4: Synthesis of 8-cyclobutyl-2-(methylthio)-7-oxo-7,8-dihydropyrido[2,3-d]pyrimidine-6-carbonitrile: To a stirred solution of 4-(cyclobutylamino)-2-(methylthio)pyrimidine-5-carbaldehyde (1000 mg, 5.33 mmol, 1 equiv) in Acetic acid (15 mL), was added Cyanoacetic acid (453 mg, 5.33 mmol, 1.2 equiv) and Benzyl amine (0.1 mL, 0.44, 0.1 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain the product. LCMS: 273 [M+H]⁺

Step—5: Synthesis of 8-cyclobutyl-2-(methylsulfinyl)-7-oxo-7,8-dihydropyrido[2,3-d]pyrimidine-6-carbonitrile: To a stirred solution of 8-cyclobutyl-2-(methylthio)-7-oxo-7,8-dihydropyrido[2,3-d]pyrimidine-6-carbonitrile (150 mg, 0.54 mmol, 1 equiv) in DCM (5 mL), was added m-CPBA (133 mg, 0.76 mmol, 1.4 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain the product. LCMS: 289 [M+H]⁺

Step—6: Synthesis of tert-butyl 6-((6-cyano-8-cyclobutyl-7-oxo-7,8-dihydropyrido[2,3-d]pyrimidin-2-yl)amino)-3,4-dihydroisoquinoline-2(1H)-carboxylate: To a stirred solution of 8-cyclobutyl-2-(methylsulfinyl)-7-oxo-7,8-dihydropyrido[2,3-d]pyrimidine-6-carbonitrile (100 mg, 0.36 mmol, 1 equiv) in toluene (5 mL), was added tert-butyl 6-amino-3,4-dihydroisoquinoline-2(1H)-carboxylate (100 mg, 0.4 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain crude compound, which was purified by recrystallization with methanol to obtain the product. LCMS: 473 [M+H]⁺

Step—7: Synthesis of 8-cyclobutyl-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-cyclobutyl-7-oxo-7,8-dihydropyrido[2,3-d]pyrimidin-2-yl)amino)-3,4-dihydroisoquinoline-2(1H)-carboxylate (60 mg, 0.127 mmol, 1 equiv) in 1.25 M HCl in ethanol (5 mL) was allowed to stir for 1 h at 50° C. Progress of the reaction was monitored by LCMS. After completion of the reaction, the resultant precipitates were filtered and washed with Ethanol to obtain the product. LCMS: 373 [M+H]⁺; ¹H NMR (DMSO-d₆, 400 MHz): δ=10.57 (br. s., 1H), 9.22 (br. s., 2H), 8.81-8.87 (m, 1H), 8.54-8.62 (m, 1H), 7.69 (br. s., 1H), 7.54 (d, J=8.3 Hz, 1H), 7.24 (d, J=8.8 Hz, 1H), 5.71-5.81 (m, 1H), 4.25 (br. s., 2H), 3.39 (br. s., 2H), 2.94-3.12 (m, 4H), 2.23 (d, J=8.3 Hz, 2H), 1.86 (br. s., 1H), 1.71-1.81 (m, 1H).

### Example—S96: Synthesis of 8-cyclopentyl-2-((1,1-dimethyl-1,2,3,4-tetrahydroisoquinolin-6-yl)amino)-7-oxo-7,8-dihydropyrido[2,3-d]pyrimidine-6-carbonitrile (Compound No. 92)

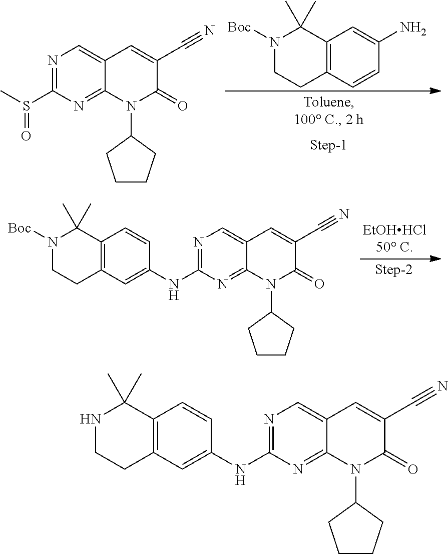

Step—1: Synthesis of tert-butyl 6-((6-cyano-8-cyclopentyl-7-oxo-7,8-dihydropyrido[2,3-d]pyrimidin-2-yl)amino)-1,1-dimethyl-3,4-dihydroisoquinoline-2(1H)-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added tert-butyl 7-amino-1,1-dimethyl-3,4-dihydroisoquinoline-2(1H)-carboxylate (91 mg, 0.33 mmol, 1.0 equiv). The resultant reaction mixture was allowed to stir at 100° C. for 2 h. Progress of the reaction was monitored by LCMS. After completion of the reaction, toluene was evaporated and the resulting solid was triturated with methanol and the precipitates were filtered to and washed with methanol to obtain the product. LCMS: 515 [M+H]⁺

Step—2: Synthesis of 8-cyclopentyl-2-((1,1-dimethyl-1,2,3,4-tetrahydroisoquinolin-6-yl)amino)-7-oxo-7,8-dihydropyrido[2,3-d]pyrimidine-6-carbonitrile: A solution of tert-butyl 6-((6-cyano-8-cyclopentyl-7-oxo-7,8-dihydropyrido[2,3-d]pyrimidin-2-yl)amino)-1,1-dimethyl-3,4-dihydroisoquinoline-2(1H)-carboxylate (50 mg, 0.097 mmol, 1 equiv) in 1.25 M HCl in ethanol (5 mL) was allowed to stir for 1 h at 50° C. Progress of the reaction was monitored by LCMS. After completion of the reaction, the resultant precipitates were filtered and washed with ethanol to obtain the product. LCMS: 415 [M+H]⁺; ¹H NMR (400 MHz, DMSO-d₆): δ=10.58 (br. s., 1H), 9.45 (br. s., 2H), 8.86 (s, 1H), 8.60 (s, 1H), 7.67 (br. s., 1H), 7.47 (br. s., 1H), 7.38-7.45 (m, 1H), 5.83 (br. s., 1H), 3.43 (br. s., 2H), 3.03 (t, J=5.7 Hz, 2H), 2.20 (br. s., 2H), 1.92 (br. s., 2H), 1.77-1.86 (m, 2H), 1.66 (s, 6H), 1.62 (d, J=6.1 Hz, 2H).

### Example—S97: Synthesis of 8-cyclopentyl-2-((1-(1-methylpiperidin-4-yl)-1H-pyrazol-4-yl) amino)-7-oxo-7, 8-dihydropyrido [2, 3-d] pyrimidine-6-carbonitrile (Compound No. 325)

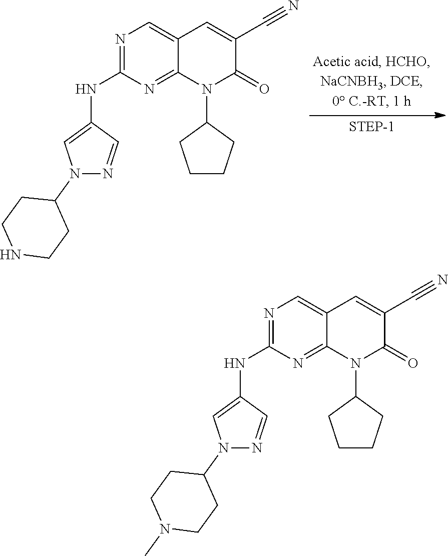 [SMILES: N#Cc1cc2cnc(Nc3cnn(C4CCNCC4)c3)nc2n(C2CCCC2)c1=O ; CN1CCC(n2cc(Nc3ncc4cc(C#N)c(=O)n(C5CCCC5)c4n3)cn2)CC1]

Step—1: Synthesis of 8-cyclopentyl-2-((1-(1-methylpiperidin-4-yl)-1H-pyrazol-4-yl)amino)-7-oxo-7,8-dihydropyrido[2,3-d]pyrimidine-6-carbonitrile: To a stirred solution of 8-cyclopentyl-7-oxo-2-((1-(piperidin-4-yl)-1H-pyrazol-4-yl)amino)-7,8-dihydropyrido[2,3-d]pyrimidine-6-carbonitrile (100 mg, 0.2 mmol, 1 equiv) in DCE (5 mL), was added Formaldehyde (40% in water) (0.04 mL, 0.6 mmol, 3 equiv) and acetic acid (0.06 mL, 1.0 mmol, 5 equiv). The reaction mixture was allowed to stir at RT for 1 h. The reaction mixture was cooled to 0° C. NaCNBH₃ (38 mg, 0.6 mmol, 3 equiv) was added to above mixture and the temperature was raised to RT. The reaction mixture was allowed to stir at RT for 1 h. Progress of the reaction was monitored by LCMS. After completion of the reaction, the reaction mixture was diluted with water (25 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 418 [M+H]⁺, ¹H NMR (400 MHz, DMSO-d₆): δ 10.32 (s, 1H), 8.78 (br. s., 1H), 8.49 (s, 1H), 7.95 (s, 1H), 7.60 (br. s., 1H), 5.76-5.94 (m, 1H), 3.92-4.16 (m, 1H), 2.85 (d, J=11.8 Hz, 2H), 2.22 (s, 5H), 2.05-1.84 (m, 10H), 1.54 (s, 2H).

### Example—S98: Synthesis of 1-cyclopentyl-2-oxo-7-((1,2,3,4-tetrahydroisoquinolin-7-yl)amino)-1,2-dihydro-1,8-naphthyridine-3-carbonitrile (Compound No. 326)

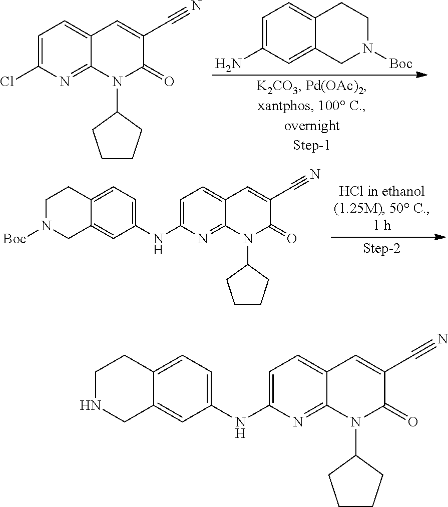

Step—1: Synthesis of tert-butyl 7-((6-cyano-8-cyclopentyl-7-oxo-7,8-dihydro-1,8-naphthyridin-2-yl)amino)-3,4-dihydroisoquinoline-2(1H)-carboxylate: To a solution of 7-chloro-1-cyclopentyl-2-oxo-1,2-dihydro-1,8-naphthyridine-3-carbonitrile (100 mg, 0.36 mmol, 1 equiv) in dioxane (5 mL), was added tert-butyl 7-amino-3,4-dihydroisoquinoline-2(1H)-carboxylate (99 mg, 0.4 mmol, 1.1 equiv) and potassium carbonate (149 mg, 1.0 mmol, 3 equiv). The reaction mixture was purged with nitrogen gas for 10 min., followed by the addition of palladium acetate (4 mg, 0.018 mmol, 0.05 equiv) and xantphos (21 mg, 0.036 mmol, 0.1 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain desired product. LCMS: 486 [M+H]⁺

Step—2: Synthesis of 1-cyclopentyl-2-oxo-7-((1,2,3,4-tetrahydroisoquinolin-7-yl)amino)-1,2-dihydro-1,8-naphthyridine-3-carbonitrile: A solution of tert-butyl 7-((6-cyano-8-cyclopentyl-7-oxo-7,8-dihydro-1,8-naphthyridin-2-yl)amino)-3,4-dihydroisoquinoline-2(1H)-carboxylate (150 mg, 0.3 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain crude, which was purified by reverse phase HPLC to obtain desired product. LCMS: 386 [M+H]⁺, ¹H NMR (DMSO-d₆, 400 MHz): δ 10.07 (s, 1H), 8.46 (s, 1H), 7.87 (d, J=8.8 Hz, 1H), 7.60 (br. s., 1H), 7.25 (d, J=7.0 Hz, 1H), 7.10 (d, J=7.9 Hz, 1H), 6.77 (d, J=8.8 Hz, 1H), 5.87-6.10 (m, 1H), 3.94 (s, 2H), 3.05 (t, J=5.7 Hz, 2H), 2.67 (br. s., 2H), 2.22 (d, J=7.9 Hz, 2H), 1.95 (br. s., 2H), 1.80 (d, J=4.8 Hz, 2H), 1.50-1.70 (m, 2H).

### Example—S99: Synthesis of 8-cyclopentyl-2-((1-(2-(dimethylamino)ethyl)-1H-pyrazol-4-yl)amino)-7-oxo-7,8-dihydropyrido[2,3-d]pyrimidine-6-carbonitrile (Compound No. 105)

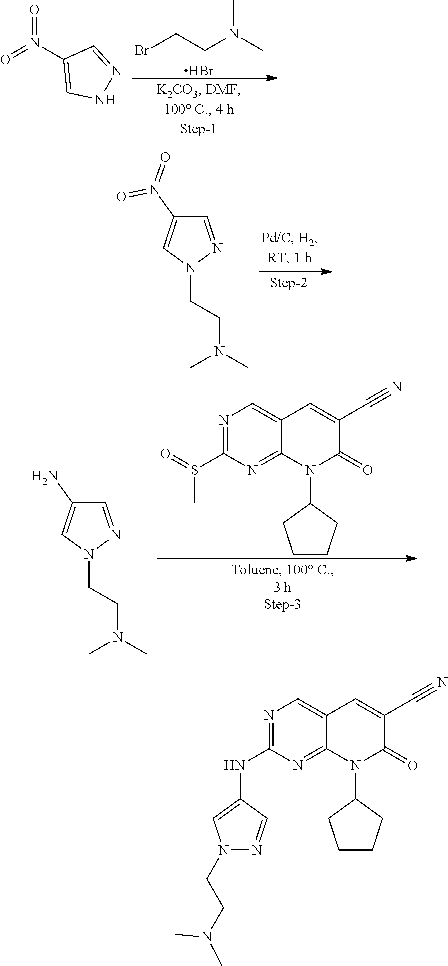

Step—1: Synthesis of N,N-dimethyl-2-(4-nitro-1H-pyrazol-1-yl)ethan-1-amine: To a stirred solution of 4-nitro-1H-pyrazole (500 mg, 4.42 mmol, 1 equiv) in DMF (5 mL), was added K₂CO₃ (1220 mg, 8.84 mmol, 2 equiv) and 2-bromo-N,N-dimethylethan-1-amine hydrobromide (1532 mg, 6.63 mmol, 1.5 equiv). The resultant reaction mixture was allowed to stir at 100° C. for 4 h. Progress of the reaction was monitored by TLC and LCMS. After completion of the reaction, diluted with water (50 mL) and extracted with ethyl acetate (150 mL). Organic layer was washed with water (50 mL) and brine solution (50 mL). Organic layer was dried over anhydrous sodium sulphate and concentrated under reduced pressure to obtain crude, which was purified by normal phase combi flash to obtain desired product. LCMS: 185 [M+H]⁺

Step—2: Synthesis of 1-(2-(dimethylamino)ethyl)-1H-pyrazol-4-amine: To a stirred solution of N,N-dimethyl-2-(4-nitro-1H-pyrazol-1-yl)ethan-1-amine (400 mg, 0.46 mmol, 1 equiv) in methanol (10 mL), was added Pd/C (20% w/w) (80 mg) under H₂ atm. The resultant reaction mixture was allowed to stir at RT for 1 h. Progress of the reaction was monitored by LCMS. After completion of the reaction, the mixture was passes through celite bed and the filtrate was concentrated under reduced pressure to obtain desired product. LCMS: 155 [M+H]⁺

Step—3: Synthesis of 8-cyclopentyl-2-((1-(2-(dimethylamino)ethyl)-1H-pyrazol-4-yl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 1-(2-(dimethylamino)ethyl)-1H-pyrazol-4-amine (55 mg, 0.36 mmol, 1.1 equiv). The resultant reaction mixture was stir at 100° C. for 3 h. Progress of the reaction was monitored by LCMS. After completion of the reaction, solvent was removed under reduced pressure to obtain crude which was purified by recrystallization with methanol to obtain desired product. LCMS: 393 [M+H]⁺, ¹H NMR (400 MHz, DMSO-d₆): δ 10.30 (br. s., 1H), 8.69-8.87 (m, 1H), 8.48 (s, 1H), 7.89 (br. s., 1H), 7.60 (s, 1H), 5.71-5.93 (m, 1H), 4.18 (t, J=6.4 Hz, 2H), 2.67 (t, J=6.4 Hz, 2H), 2.13-2.28 (m, 8H), 1.98 (br. s., 2H), 1.85 (br. s., 2H), 1.64 (d, J=3.9 Hz, 2H).

### Example—S100: Synthesis of 8-cyclopentyl-2-((1-(2-methoxyethyl)-1H-pyrazol-4-yl) amino)-7-oxo-7, 8-dihydropyrido [2, 3-d] pyrimidine-6-carbonitrile (Compound No. 104)

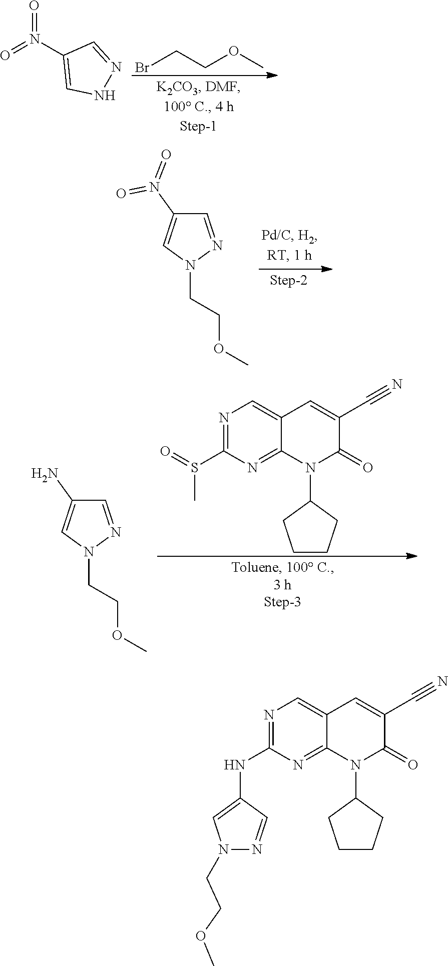

Step—1: Synthesis of 1-(2-methoxyethyl)-4-nitro-1H-pyrazole: To a stirred solution of 4-nitro-1H-pyrazole (500 mg, 4.42 mmol, 1 equiv) in DMF (5 mL), was added K₂CO₃ (1220 mg, 8.84 mmol, 2 equiv) and 1-bromo-2-methoxyethane (915 mg, 6.63 mmol, 1.5 equiv). The resultant reaction mixture was allowed to stir at 100° C. for 4 h. Progress of the reaction was monitored by TLC and LCMS. After completion of the reaction, diluted with water (50 mL) and extracted with ethyl acetate (150 mL). Organic layer was washed with water (50 mL) and brine solution (50 mL). Organic layer was dried over anhydrous sodium sulphate and concentrated under reduced pressure to obtain desired product. LCMS: 172 [M+H]⁺

Step—2: Synthesis of 1-(2-methoxyethyl)-1H-pyrazol-4-amine: To a stirred solution of 1-(2-methoxyethyl)-4-nitro-1H-pyrazole (500 mg, 2.9 mmol, 1 equiv) in methanol (10 mL), was added Pd/C (20% w/w) (100 mg) under H₂ atm. The resultant reaction mixture was allowed to stir at RT for 1 h. Progress of the reaction was monitored by LCMS. After completion of the reaction, the mixture was passes through celite bed and the filtrate was concentrated under reduced pressure to obtain desired product. LCMS: 142 [M+H]⁺

Step—3: Synthesis of 8-cyclopentyl-2-((1-(2-methoxyethyl)-1H-pyrazol-4-yl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 1-(2-methoxyethyl)-1H-pyrazol-4-amine (51 mg, 0.36 mmol, 1.1 equiv). The resultant reaction mixture was stir at 100° C. for 3 h. Progress of the reaction was monitored by LCMS. After completion of the reaction, solvent was removed under reduced pressure to obtain crude which was purified by recrystallization with methanol to obtain desired product. LCMS: 380 [M+H]⁺, ¹H NMR (400 MHz, DMSO-d₆): δ10.33 (br. s., 1H), 8.78 (br. s., 1H), 8.49 (s, 1H), 7.89 (br. s., 1H), 7.61 (s, 1H), 5.64-5.93 (m, 1H), 4.25 (t, J=5.5 Hz, 2H), 3.70 (t, J=5.3 Hz, 2H), 3.26 (s, 3H), 2.33 (br. s., 2H), 1.98 (br. s., 2H), 1.85 (br. s., 2H), 1.65 (br. s., 2H).

### Example—S101: Synthesis of 8-cyclopentyl-2-((1-(1-(dimethylglycyl)piperidin-4-yl)-1H-pyrazol-4-yl)amino)-7-oxo-7,8-dihydropyrido[2,3-d]pyrimidine-6-carbonitrile (Compound No. 202)

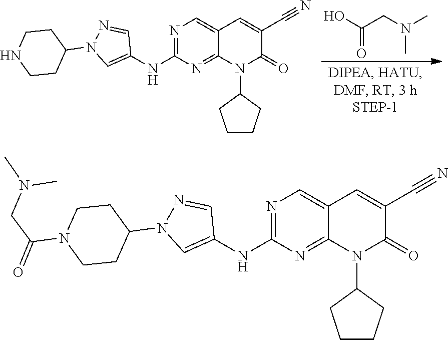

Step—1: Synthesis of 8-cyclopentyl-2-((1-(1-(dimethylglycyl)piperidin-4-yl)-1H-pyrazol-4-yl)amino)-7-oxo-7,8-dihydropyrido[2,3-d]pyrimidine-6-carbonitrile: To a stirred solution of 8-cyclopentyl-7-oxo-2-((1-(piperidin-4-yl)-1H-pyrazol-4-yl)amino)-7,8-dihydropyrido[2,3-d]pyrimidine-6-carbonitrile (100 mg, 0.22 mmol, 1 equiv) in DMF (3 mL), was added dimethyl glycine (23 mg, 0.22 mmol, 1 equiv), DIPEA (0.2 mL, 0.88 mmol, 4 equiv) and HATU (151 mg, 0.39 mmol, 1.8 equiv). The reaction mixture was allowed to stir for 3 h at RT. Progress of the reaction was monitored by LCMS. After completion of the reaction, the reaction mixture was diluted with water (25 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 490 [M+H]⁺, ¹H NMR (DMSO-d₆, 400 MHz): δ 10.21 (br. s., 1H), 8.78 (br. s., 1H), 8.49 (s, 1H), 7.95 (br. s., 1H), 7.62 (s, 1H), 5.83 (m, 1H), 4.34-4.52 (m, 1H), 4.22 (br. s., 2H), 2.81 (br. s., 2H), 2.22 (s, 8H), 2.09 (br. s., 2H), 1.96-1.77 (br. s., 8H), 1.62 (br. s., 2H).

### Example—S102: Synthesis of 2-(benzo[d]thiazol-2-ylamino)-8-cyclopentyl-7-oxo-7,8-dihydropyrido[2,3-d]pyrimidine-6-carbonitrile (Compound No. 100)

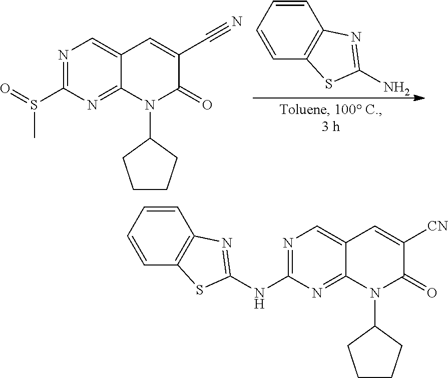

To a stirred solution of 8-cyclopentyl-2-(methylsulfinyl)-7-oxo-7,8-dihydropyrido[2,3-d]pyrimidine-6-carbonitrile (150 mg, 0.50 mmol, 1 equiv) in toluene (5 mL), was added benzo[d]thiazol-2-amine (82 mg, 0.55 mmol, 1.1 equiv). The resultant reaction mixture was stir at 100° C. for 3 h. Progress of the reaction was monitored by LCMS. After completion of the reaction, solvent was removed under reduced pressure to obtain crude which was purified by reverse phase HPLC obtain desired product. LCMS: 389 [M+H]⁺, ¹H NMR (DMSO-d₆, 400 MHz): δ 12.75 (br. s., 1H), 9.04 (s, 1H), 8.71 (s, 1H), 8.01 (d, J=7.9 Hz, 1H), 7.76 (d, J=7.9 Hz, 1H), 7.46 (t, J=7.2 Hz, 1H), 7.32 (t, J=7.2 Hz, 1H), 2.25 (dd, J=11.4, 7.9 Hz, 2H), 2.05 (br. s., 2H), 1.95 (br. s., 2H), 1.72 (br. s., 2H).

### Example—S103: Synthesis of 8-cyclopentyl-7-oxo-2-((4-(piperidin-4-yl) thiazol-2-yl) amino)-7, 8-dihydropyrido [2, 3-d] pyrimidine-6-carbonitrile (Compound No. 327)

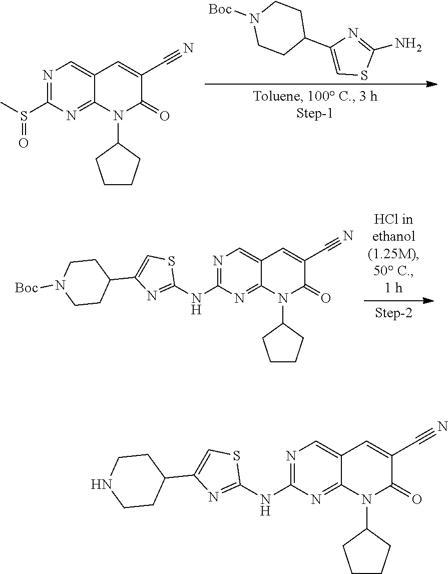 [SMILES: CS(=O)c1ncc2cc(C#N)c(=O)n(C3CCCC3)c2n1 ; CC(C)(C)OC(=O)N1CCC(c2csc(N)n2)CC1 ; CC(C)(C)OC(=O)N1CCC(c2csc(Nc3ncc4cc(C#N)c(=O)n(C5CCCC5)c4n3)n2)CC1 ; N#Cc1cc2cnc(Nc3nc(C4CCNCC4)cs3)nc2n(C2CCCC2)c1=O]

Step—1: Synthesis of tert-butyl 4-(2-((6-cyano-8-cyclopentyl-7-oxo-7,8-dihydropyrido[2,3-d]pyrimidin-2-yl)amino)thiazol-4-yl)piperidine-1-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added tert-butyl 4-(2-aminothiazol-4-yl)piperidine-1-carboxylate (102 mg, 0.36 mmol, 1.1 equiv). The resultant reaction mixture was allowed to stir at 100° C. for 3 h. Progress of the reaction was monitored by LCMS. After completion of the reaction, solvent was removed under reduced pressure to obtain crude, which was purified by trituration with methanol to obtain desired product. LCMS: 522 [M+H]⁺

Step—2: Synthesis of 8-cyclopentyl-7-oxo-2-((4-(piperidin-4-yl)thiazol-2-yl)amino)-7,8-dihydropyrido[2,3-d]pyrimidine-6-carbonitrile: A solution of tert-butyl 4-(2-((6-cyano-8-cyclopentyl-7-oxo-7,8-dihydropyrido[2,3-d]pyrimidin-2-yl)amino)thiazol-4-yl)piperidine-1-carboxylate (100 mg, 0.19 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain residue, which was dried under lyophilizer to obtain desired product. LCMS: 422 [M+H]⁺, ¹H NMR (DMSO-d₆, 400 MHz): δ 12.45 (s, 1H), 9.08 (br. s., 1H), 8.97 (s, 1H), 8.92 (d, J=8.3 Hz, 1H), 8.68 (s, 1H), 7.01 (br. s., 1H), 6.06 (br. s., 2H), 3.30 (d, J=12.0 Hz, 2H), 2.89-3.06 (m, 3H), 2.19 (d, J=7.0 Hz, 2H), 2.08 (br. s, 2H), 2.01 (br. s., 2H), 1.77-1.94 (m, 4H), 1.66 (br. s., 2H).

### Example—S104: Synthesis of 8-cyclopentyl-7-oxo-2-((3-(piperidin-4-yl)-1H-indazol-5-yl) amino)-7, 8-dihydropyrido [2, 3-d] pyrimidine-6-carbonitrile (Compound No. 169)

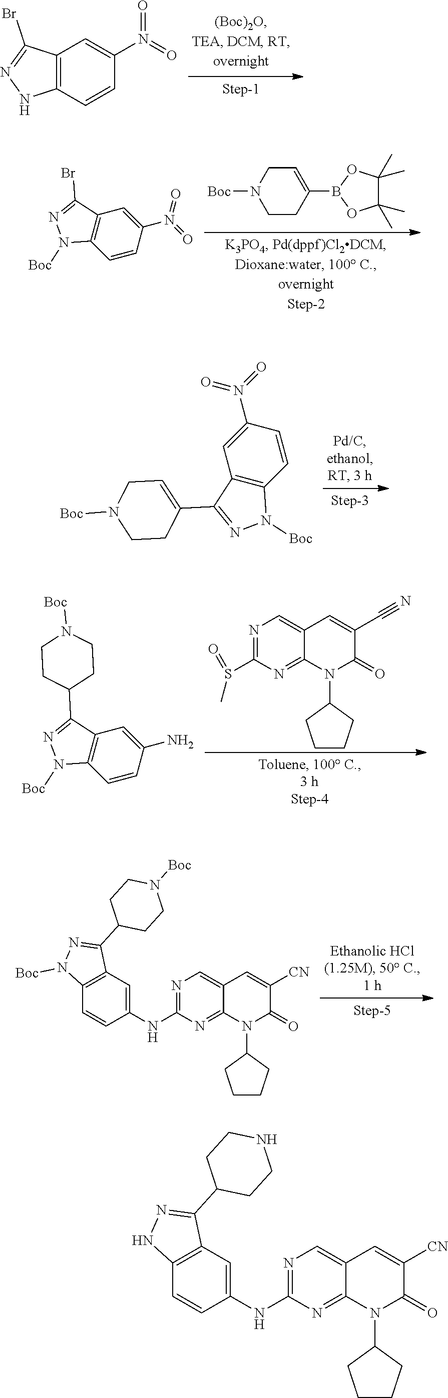

Step—1: Synthesis of tert-butyl 3-bromo-5-nitro-1H-indazole-1-carboxylate: To a stirred solution of 3-bromo-5-nitro-1H-indazole (1000 mg, 4.14 mmol, 1 equiv) in DCM (15 mL), was added TEA (1.2 mL, 8.26 mmol, 2 equiv) and boc anhydride (1 mL, 4.56 mmol, 1.1 equiv). The resultant reaction mixture was allowed to stir at RT for overnight. Progress of the reaction was monitored by TLC and LCMS. After completion of the reaction, diluted with DCM (150 mL) and washed with water (100 mL), brine solution (100 mL). Organic layer was dried over anhydrous sodium sulphate and concentrated under reduced pressure to obtain desired product. LCMS: 342 [M+H]⁺

Step—2: Synthesis of tert-butyl 3-(1-(tert-butoxycarbonyl)-1,2,3,6-tetrahydropyridin-4-yl)-5-nitro-1H-indazole-1-carboxylate: To a solution of tert-butyl 3-bromo-5-nitro-1H-indazole-1-carboxylate (1000 mg, 2.9 mmol, 1 equiv) in dioxane (10 mL), was added tert-butyl 4-(4,4,5,5-tetramethyl-1,3,2-dioxaborolan-2-yl)-3,6-dihydropyridine-1(2H)-carboxylate (1359 mg, 1.5 mmol, 1 equiv) and a solution of potassium phosphate (1537 mg, 7.25 mmol, 2.5 equiv) in water (2 mL). The reaction mixture was purged with nitrogen gas for 15 min., followed by the addition of Pd(dppf)C₁₂.DCM (118 mg, 0.15 mmol, 0.05 equiv). The resultant reaction mixture was allowed to stir at 100° C. for overnight. Progress of the reaction was monitored by TLC and LCMS. After completion of the reaction, reaction mixture was diluted with water (100 mL) and extracted with ethyl acetate (150 mL). Organic layer was washed with water (100 mL) and brine solution (100 mL). Organic layer was dried over anhydrous sodium sulphate and concentrated under reduced pressure to obtain crude, which was purified by normal phase combi flash to obtain desired product. LCMS: 445 [M+H]⁺

Step—3: Synthesis of tert-butyl 5-amino-3-(1-(tert-butoxycarbonyl)piperidin-4-yl)-1H-indazole-1-carboxylate: To a stirred solution of tert-butyl 3-(1-(tert-butoxycarbonyl)-1,2,3,6-tetrahydropyridin-4-yl)-5-nitro-1H-indazole-1-carboxylate (400 mg, 0.9 mmol, 1 equiv) in ethanol (10 mL), was added Pd/C (20% w/w) (80 mg) under H₂ atm. The resultant reaction mixture was allowed to stir at RT for 3 h. Progress of the reaction was monitored by LCMS. After completion of the reaction, the mixture was passes through celite bed and the filtrate was concentrated under reduced pressure to obtain desired product. LCMS: 417 [M+H]⁺

Step—4: Synthesis of tert-butyl 3-(1-(tert-butoxycarbonyl)piperidin-4-yl)-5-((6-cyano-8-cyclopentyl-7-oxo-7,8-dihydropyrido[2,3-d]pyrimidin-2-yl)amino)-1H-indazole-1-carboxylate: To a stirred solution of tert-butyl 5-amino-3-(1-(tert-butoxycarbonyl)piperidin-4-yl)-1H-indazole-1-carboxylate (100 mg, 0.33 mmol, 1 equiv) in toluene (5 mL), was added tert-butyl 4-(4-amino-1H-pyrazol-1-yl) piperidine-1-carboxylate (150 mg, 0.36 mmol, 1.1 equiv). The resultant reaction mixture was stir at 100° C. for 1 h. Progress of the reaction was monitored by LCMS. After completion of the reaction, solvent was removed under reduced pressure to obtain crude which was purified by recrystallization with methanol to obtain desired product. LCMS: 655 [M+H]⁺

Step—5: Synthesis of 8-cyclopentyl-7-oxo-2-((3-(piperidin-4-yl)-1H-indazol-5-yl)amino)-7,8-dihydropyrido[2,3-d]pyrimidine-6-carbonitrile: tert-butyl 3-(1-(tert-butoxycarbonyl)piperidin-4-yl)-5-((6-cyano-8-cyclopentyl-7-oxo-7,8-dihydropyrido[2,3-d]pyrimidin-2-yl)amino)-1H-indazole-1-carboxylate (180 mg, 0.27 mmol, 1 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure and the residue was purified by reverse phase HPLC to obtain desired product. LCMS: 455 [M+H]⁺; ¹H NMR (400 MHz, DMSO-d₆): δ 12.72 (br. s., 1H), 10.51 (br. s., 1H), 8.83 (s, 1H), 8.56 (s, 1H), 8.06 (s, 1H), 7.26-7.52 (m, 2H), 5.78 (br. s., 1H), 3.14-3.17 (m, 2H), 2.74 (br. s., 2H), 2.11 (s, 2H), 1.92 (br. s., 2H), 1.83 (s, 3H), 1.86 (s, 3H), 1.75 (s, 2H), 1.43 (br. s., 2H).

### Example—S105: Synthesis of 8-cyclopentyl-2-((3-methyl-1-(piperidin-4-yl)-1H-pyrazol-4-yl)amino)-7-oxo-7,8-dihydropyrido[2,3-d]pyrimidine-6-carbonitrile (Compound No. 328)

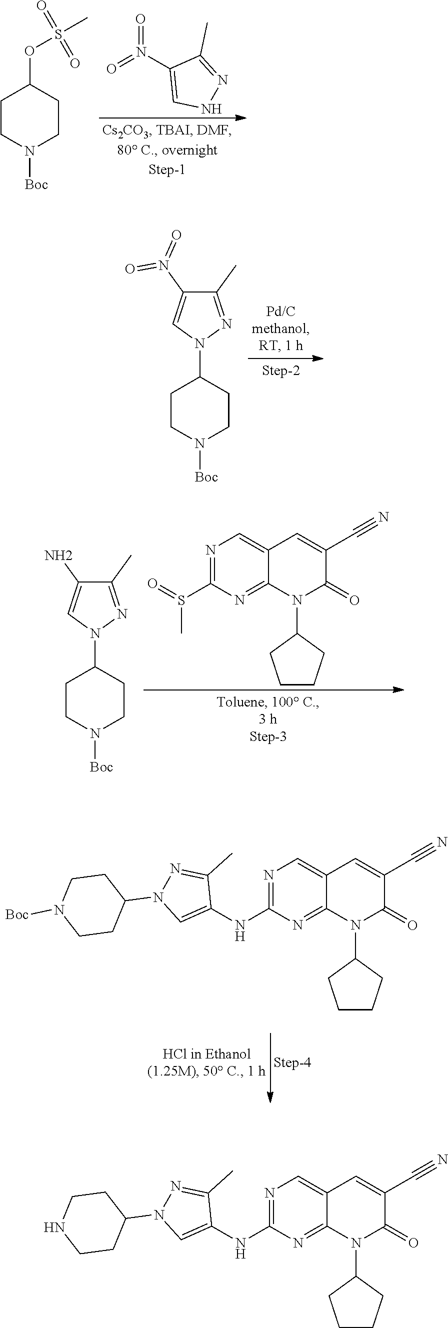

Step—1: Synthesis of tert-butyl 4-(3-methyl-4-nitro-1H-pyrazol-1-yl)piperidine-1-carboxylate: To a stirred solution of tert-butyl 4-((methylsulfonyl)oxy)piperidine-1-carboxylate (500 mg, 1.79 mmol, 1 equiv) in DMF (10 mL), was added Cs₂CO₃ (1167 mg, 3.58 mmol, 2 equiv), TBAI (133 mg, 0.36 mmol, 0.2 equiv) and 3-methyl-4-nitro-1H-pyrazole (114 mg, 0.89 mmol, 0.5 equiv). The resultant reaction mixture was allowed to stir at 80° C. for overnight. Progress of the reaction was monitored by LCMS. After completion of the reaction, diluted with water (50 mL) and extracted with EtOAc (100 mL×2). Organic layer was washed with water (100 mL) and brine solution (100 mL). Organic layer was dried over anhydrous sodium sulphate and concentrated under reduced pressure to obtain crude, which was purified by normal phase Combi flash to obtain desired product. LCMS: 311 [M+H]⁺

Step—2: Synthesis of tert-butyl 4-(4-amino-3-methyl-1H-pyrazol-1-yl)piperidine-1-carboxylate: To a stirred solution of tert-butyl 4-(3-methyl-4-nitro-1H-pyrazol-1-yl) piperidine-1-carboxylate (200 mg, 0.67 mmol, 1 equiv) in methanol (10 mL), was added Pd/C (20% w/w) (40 mg). The resultant reaction mixture was allowed to stir at RT for 1 h. Progress of the reaction was monitored by TLC and LCMS. After completion of the reaction, the mixture was passes through celite bed and the filtrate was concentrated under reduced pressure to obtain desired product. LCMS: 281 [M+H]⁺

Step—3: Synthesis of tert-butyl 4-(4-((6-cyano-8-cyclopentyl-7-oxo-7,8-dihydropyrido[2,3-d]pyrimidin-2-yl)amino)-3-methyl-1H-pyrazol-1-yl)piperidine-1-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added tert-butyl 4-(4-amino-3-methyl-1H-pyrazol-1-yl)piperidine-1-carboxylate (101 mg, 0.36 mmol, 1.1 equiv). The resultant reaction mixture was stir at 100° C. for 3 h. Progress of the reaction was monitored by LCMS. After completion of the reaction, solvent was removed under reduced pressure to obtain crude which was purified by recrystallization with methanol to obtain desired product. LCMS: 519 [M+H]⁺

Step—4: Synthesis of 8-cyclopentyl-2-((3-methyl-1-(piperidin-4-yl)-1H-pyrazol-4-yl)amino)-7-oxo-7,8-dihydropyrido[2,3-d]pyrimidine-6-carbonitrile: tert-butyl 4-(4-((6-cyano-8-cyclopentyl-7-oxo-7,8-dihydropyrido[2,3-d]pyrimidin-2-yl)amino)-3-methyl-1H-pyrazol-1-yl)piperidine-1-carboxylate (45 mg, 0.086 mmol, 1 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure and the residue was dried under Lyophiliser to obtain desired product. LCMS: 419 [M+H]⁺, 1H NMR: (400 MHz, DMSO-d6) δ 9.52 (br. s., 1H), 8.74 (s, 1H), 8.46 (s, 1H), 7.49 (s, 1H), 5.60 (br. s., 1H), 4.17 (br. s., 1H), 3.07 (d, J=9.2 Hz, 2H), 2.67 (s, 2H), 2.20 (s, 3H), 1.81-1.99 (m, 5H), 1.76 (d, J=9.6 Hz, 4H), 1.68 (br. s., 2H), 1.47 (br. s., 2H).

### Example—S106: Synthesis of 1-cyclopentyl-7-((1-(methylsulfonyl)piperidin-4-yl)amino)-2-oxo-1,2-dihydro-1,6-naphthyridine-3-carbonitrile (Compound No. 303)

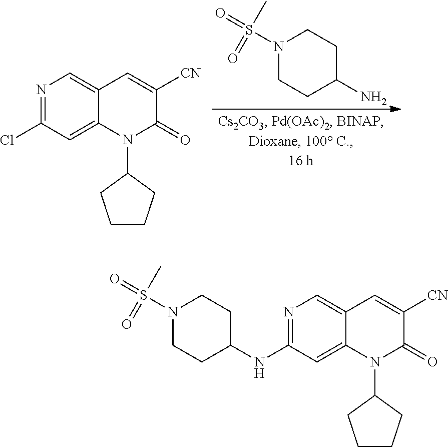

To a solution of 7-chloro-1-cyclopentyl-2-oxo-1,2-dihydro-1,6-naphthyridine-3-carbonitrile (120 mg, 0.58 mmol, 1 equiv) in dioxane (5 mL), was added 1-(methylsulfonyl)piperidin-4-amine (100 mg, 0.58 mmol, 1.0 equiv) and cesium carbonate (270 mg, 0.87 mmol, 1.5 equiv). The reaction mixture was purged with nitrogen gas for 10 min., followed by the addition of palladium acetate (6.3 mg, 0.029 mmol, 0.05 equiv) and BINAP (35 mg, 0.058 mmol, 0.1 equiv). The resultant reaction mixture was allowed to stir at 100° C. for overnight. Progress of the reaction was monitored by TLC and LCMS. After completion of the reaction, filtered through Celite pad, volatiles were removed in vacuum. Crude was purified using SFC to obtain the desired product. LCMS: 416 [M+H]⁺, 1H NMR (DMSO-d₆, 400 MHz): δ 8.47 (s, 1H), 8.46 (s, 1H), 7.69 (d, J=7.5 Hz, 2H), 6.39 (br. s., 1H), 5.22 (d, J=9.6 Hz, 1H), 3.54 (d, J=6.6 Hz, 2H), 2.90-2.96 (m, 2H), 2.89 (s, 3H), 1.92-2.16 (m, 6H), 1.85 (d, J=6.6 Hz, 2H), 1.65 (br. s., 2H), 1.44-1.59 (m, 2H).

### Example—S107: Synthesis of 8-cyclopentyl-2-((3-(1-methylpiperidin-4-yl)-1H-indazol-5-yl) amino)-7-oxo-7, 8-dihydropyrido [2, 3-d] pyrimidine-6-carbonitrile (Compound No. 170)

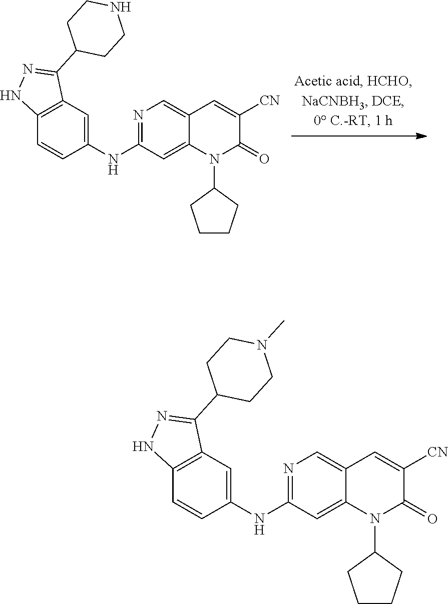 [SMILES: N#Cc1cc2cnc(Nc3ccc4[nH]nc(C5CCNCC5)c4c3)cc2n(C2CCCC2)c1=O ; CN1CCC(c2n[nH]c3ccc(Nc4cc5c(cn4)cc(C#N)c(=O)n5C4CCCC4)cc23)CC1]

To a stirred solution of 8-cyclopentyl-7-oxo-2-((3-(piperidin-4-yl)-1H-indazol-5-yl)amino)-7,8-dihydropyrido[2,3-d]pyrimidine-6-carbonitrile (50 mg, 0.11 mmol, 1 equiv) in DCE (3 mL), was added Formaldehyde (40% in water) (10 mg, 0.33 mmol, 3 equiv), acetic acid (33 mg, 0.55 mmol, 5 equiv). The reaction mixture was allowed to stir at RT for 1 h. The reaction mixture was cooled to 0° C. NaCNBH₃ (21 mg, 0.33 mmol, 3 equiv) was added to above mixture and raise the temperature to RT. The reaction mixture was allowed to stir at RT for 1 h. Progress of the reaction was monitored by LCMS. After completion of the reaction, the reaction mixture was diluted with water (15 mL) and extracted with ethyl acetate (50 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 469 [M+H]⁺, ¹H NMR (400 MHz, DMSO-d₆): δ 12.70 (br. s., 1H), 10.52 (br. s., 1H), 8.83 (s, 1H), 8.55 (s, 1H), 8.09 (br. s., 1H), 7.44-7.47 (br. s, 2H), 5.83 (br. s., 1H), 2.92 (s, 3H), 2.23 (s, 3H), 2.12 (br. s., 4H), 1.91 (br. s., 4H), 1.77 (br. s., 4H), 1.48 (br. s., 2H).

### Example—S108: Synthesis of 8-(bicyclo[3.1.0]hexan-3-yl)-7-oxo-2-((1,2,3,4-tetrahydroisoquinolin-6-yl)amino)-7,8-dihydropyrido[2,3-d]pyrimidine-6-carbonitrile (Compound No. 342)

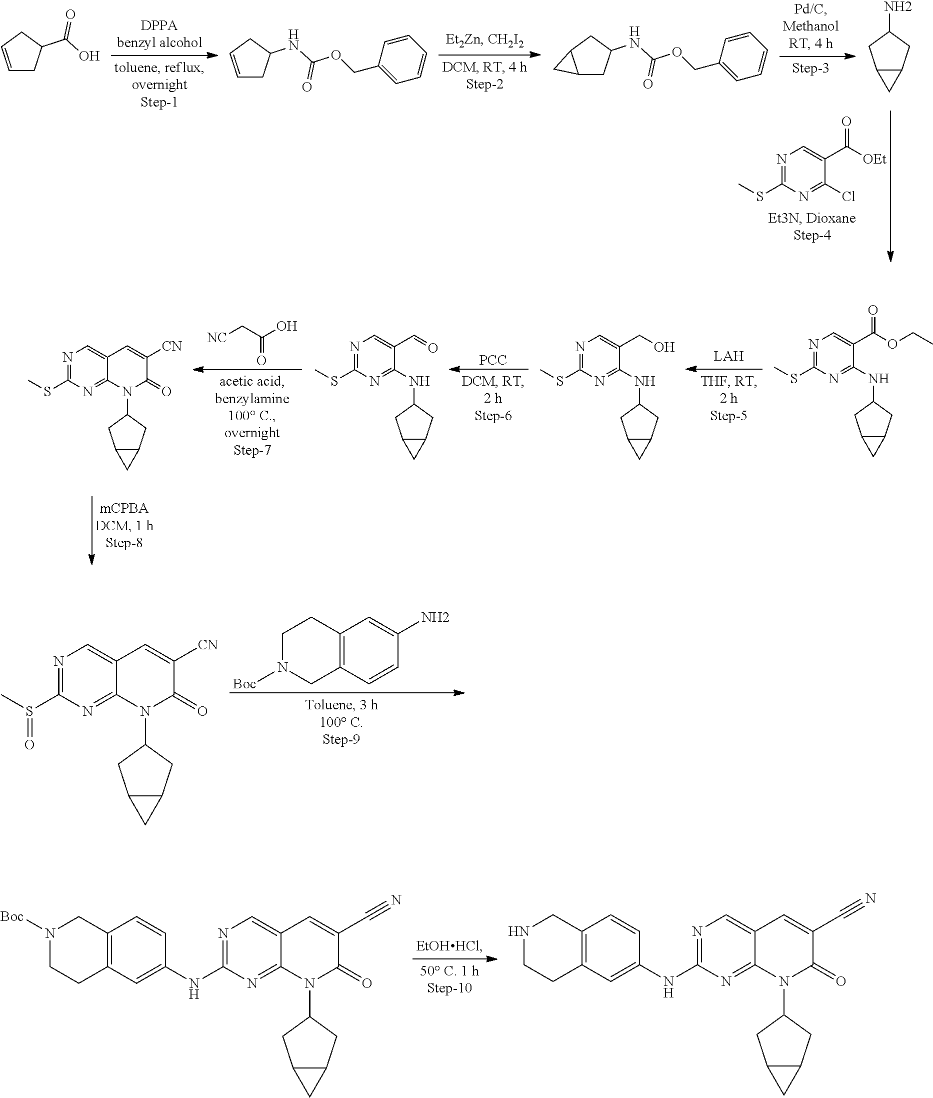

Step—1: Synthesis of benzyl cyclopent-3-en-1-ylcarbamate: To a solution of cyclopent-3-ene-1-carboxylic acid (5 g, 44.6 mmol, 1.0 eq) and DPPA (13.5 g, 49 mmol, 1.1 equiv) in toluene (80 mL) was added Et₃N (7.4 mL, 53.5 mmol, 1.2 equiv) at RT. The mixture was then stirred at reflux for 2H. Benzyl alcohol (7 mL, 66.9 mmol, 1.5 eq) was then added at RT, the resulting mixture was stirred at 100° C. overnight and cooled to room temperature. The reaction mixture was quenched with saturated aqueous NaHCO₃. The resulting mixture was extracted with EtOAc. Combined organic layers were washed with brine, dried over anhydrous Na2SO₄, and concentrated under reduced pressure. The residue was purified by flash chromatography using PE/EtOAc (5:1) as eluent to give the desired product.

Step—2: Synthesis of benzyl bicyclo[3.1.0]hexan-3-ylcarbamate: To a solution of benzyl cyclopent-3-en-1-ylcarbamate (4.0 g, 18.41 mmol, 1.0 equiv) in DCM (30 ml) was added ZnEt₂ (1 M, 27.61 ml, 27.61 mmol) followed by the addition of CH₂I₂ (2.23 ml, 27.61 mmol, 1.5 equiv) at 0° C. The reaction mixture was allowed to warm to RT and stirred for 4H. The resulting mixture was washed with brine, dried over Na2SO₄, filtered and the solvent was concentrated. The residue was purified via flash chromatography to give the desired product.

Step—3: Synthesis of bicyclo[3.1.0]hexan-3-amine: To a solution of benzyl bicyclo [3.1.0]hexan-3-ylcarbamate (2 g, 8.66 mmol, 1.0 equiv) in MeOH (20 mL) at RT under an atmosphere of nitrogen was added Pd/C (0.2 g) in one portion. The resulting mixture was then stirred under a hydrogen balloon overnight. The reaction mixture was filtered and the filtrate was concentrated under reduced pressure to give the desired product which was used directly in the next step without any further purification.

Step—4: Synthesis of ethyl 4-(bicyclo[3.1.0]hexan-3-ylamino)-2-(methylthio)pyrimidine-5-carboxylate: To a stirred solution of ethyl 4-chloro-2-(methylthio)pyrimidine-5-carboxylate (2.0 g, 8.58 mmol, 1 equiv) in Dioxane (20 mL), was added Et₃N (3.6 mL, 10.3 mmol, 1.2 equiv) and bicyclo[3.1.0]hexan-3-amine (1.00 g, 10.31 mmol, 1.2 equiv) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain the product.

Step—5: Synthesis of (4-(bicyclo[3.1.0]hexan-3-ylamino)-2-(methylthio)pyrimidin-5-yl)methanol: To a stirred solution of Ethyl 4-(bicyclo[3.1.0]hexan-3-ylamino)-2-(methylthio)pyrimidine-5-carboxylate (2.0 g, 6.82 mmol, 1.0 equiv) in THF (20 mL), was added LAH (518 mg, 13.64 mmol, 2 equiv) at 0° C. The reaction mixture was allowed to stir at RT for 3 h. Progress of the reaction was monitored by TLC and LCMS. After completion of the reaction, the reaction mixture was quenched with water (10 ml) followed by the addition of 10% solution of sodium hydroxide (5 mL) at 0° C. and stirred for 10 min at RT. The resulting mixture was filtered through celite and dried over anhydrous sodium sulphate and concentrated under reduced pressure to obtain the product.

Step—6: Synthesis of 4-(bicyclo[3.1.0]hexan-3-ylamino)-2-(methylthio)pyrimidine-5-carbaldehyde: To a stirred solution of (4-(bicyclo[3.1.0]hexan-3-ylamino)-2-(methylthio)pyrimidin-5-yl)methanol (1.40 g, 5.57 mmol, 1 equiv) in DCM (20 mL), was added PCC (1.32 g, 6.13 mmol, 1.1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain the product.

Step—7: Synthesis of 8-(bicyclo[3.1.0]hexan-3-yl)-2-(methylthio)-7-oxo-7,8-dihydropyrido[2,3-d]pyrimidine-6-carbonitrile: To a stirred solution of 4-(bicyclo[3.1.0]hexan-3-ylamino)-2-(methylthio)pyrimidine-5-carbaldehyde (1.0 g, 4.01 mmol, 1.0 equiv) in Acetic acid (15 mL), was added Cyanoacetic acid (409 mg, 4.81 mmol, 1.2 equiv) and Benzyl amine (0.044 mL, 0.40, 0.1 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain the product.

Step—8: Synthesis of 8-(bicyclo[3.1.0]hexan-3-yl)-2-(methylsulfinyl)-7-oxo-7,8-dihydropyrido[2,3-d]pyrimidine-6-carbonitrile: To a stirred solution of 8-(bicyclo[3.1.0]hexan-3-yl)-2-(methylthio)-7-oxo-7,8-dihydropyrido[2,3-d]pyrimidine-6-carbonitrile (400 mg, 1.34 mmol, 1 equiv) in DCM (10 mL), was added m-CPBA (324 mg, 0.76 mmol, 1.9 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain the product.

Step—9: Synthesis of tert-butyl 6-((8-(bicyclo[3.1.0]hexan-3-yl)-6-cyano-7-oxo-7,8-dihydropyrido[2,3-d]pyrimidin-2-yl)amino)-3,4-dihydroisoquinoline-2(1H)-carboxylate: To a stirred solution of 8-(bicyclo[3.1.0]hexan-3-yl)-2-(methylsulfinyl)-7-oxo-7,8-dihydropyrido[2,3-d]pyrimidine-6-carbonitrile (200 mg, 0.64 mmol, 1 equiv) in toluene (5 mL), was added tert-butyl 6-amino-3,4-dihydroisoquinoline-2(1H)-carboxylate (158 mg, 0.64 mmol, 1.1 equiv). The resultant reaction mixture was allowed to stir at 100° C. for 3 h. Progress of the reaction was monitored by LCMS. After completion of the reaction, solid observed was filtered and dried under vacuum to obtain crude compound, which was purified by recrystallization with methanol to obtain the desired product.

Step—10: Synthesis of 8-(bicyclo[3.1.0]hexan-3-yl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((8-(bicyclo[3.1.0]hexan-3-yl)-6-cyano-7-oxo-7,8-dihydropyrido[2,3-d]pyrimidin-2-yl)amino)-3,4-dihydroisoquinoline-2(1H)-carboxylate (80 mg, 0.16 mmol, 1 equiv) in 1.25 M HCl in ethanol (5 mL) was allowed to stir for 1 h at 50° C. Progress of the reaction was monitored by LCMS. After completion of the reaction, the resultant precipitates were filtered and washed with Ethanol to obtain the product. LCMS: 399 [M+H]⁺; ¹H NMR (400 MHz, DMSO-d₆) δ ppm 10.63 (br s, 1H) 9.33 (br s, 2H) 8.86 (s, 1H) 8.59 (s, 1H) 7.75 (br s, 1H) 7.47 (br s, 1H) 7.23 (d, J=7.89 Hz, 1H) 6.14 (br s, 1H) 3.38 (br s, 2H) 3.02 (br s, 2H) 2.11 (d, J=8.33 Hz, 4H) 1.39 (br s, 2H) 0.85 (br s, 1H) 0.70 (br s, 1H)

### Example—S109: Synthesis of 8-(bicyclo[3.1.0]hexan-3-yl)-2-((1-(methylsulfonyl)piperidin-4-yl)amino)-7-oxo-7,8-dihydropyrido[2,3-d]pyrimidine-6-carbonitrile (Compound No. 341)

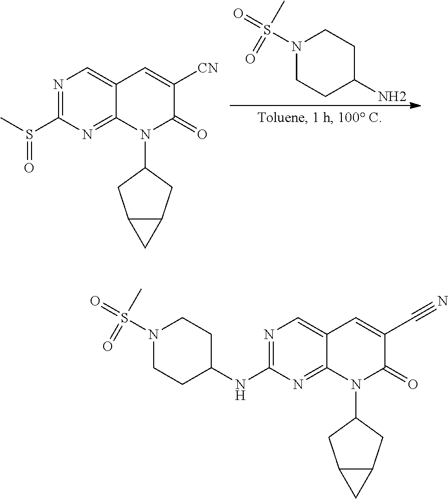

To the solution of 8-(bicyclo[3.1.0]hexan-3-yl)-2-(methylsulfinyl)-7-oxo-7,8-dihydropyrido[2,3-d]pyrimidine-6-carbonitrile (0.1 g, 0.32 mmol, 1.0 equiv) in Toluene (5 mL) was added 1-(methylsulfonyl)piperidin-4-amine (57 mg, 0.32 mmol, 1.0 equiv). The reaction mixture was then stirred at 100° C. for 1 h until the completion of the reaction mixture. After completion of the reaction, toluene was evaporated under vacuum and product was purified using reverse phase HPLC to give the desired product. LCMS: 429.1 [M+H]⁺; ¹H NMR (400 MHz, DMSO-d₆, D20) δ ppm 8.64-8.70 (s, 1H) 8.39 (s, 1H) 6.01-6.21 (m, 1H) 3.90-4.10 (m, 1H) 3.54 (d, J=12.2 Hz, 2H) 2.77-2.98 (m, 4H) 2.00-2.23 (m, 5H) 1.95 (d, J=18.8 Hz, 2H) 1.60 (td, J=19.6, 10.7 Hz, 2H) 1.26-1.41 (m, 2H) 0.74-0.84 (m, 2H).

### Example—S110: Synthesis of 8-(3-ethoxypropyl)-7-oxo-2-((1,2,3,4-tetrahydroisoquinolin-6-yl)amino)-7,8-dihydropyrido[2,3-d]pyrimidine-6-carbonitrile (Compound No. 343)

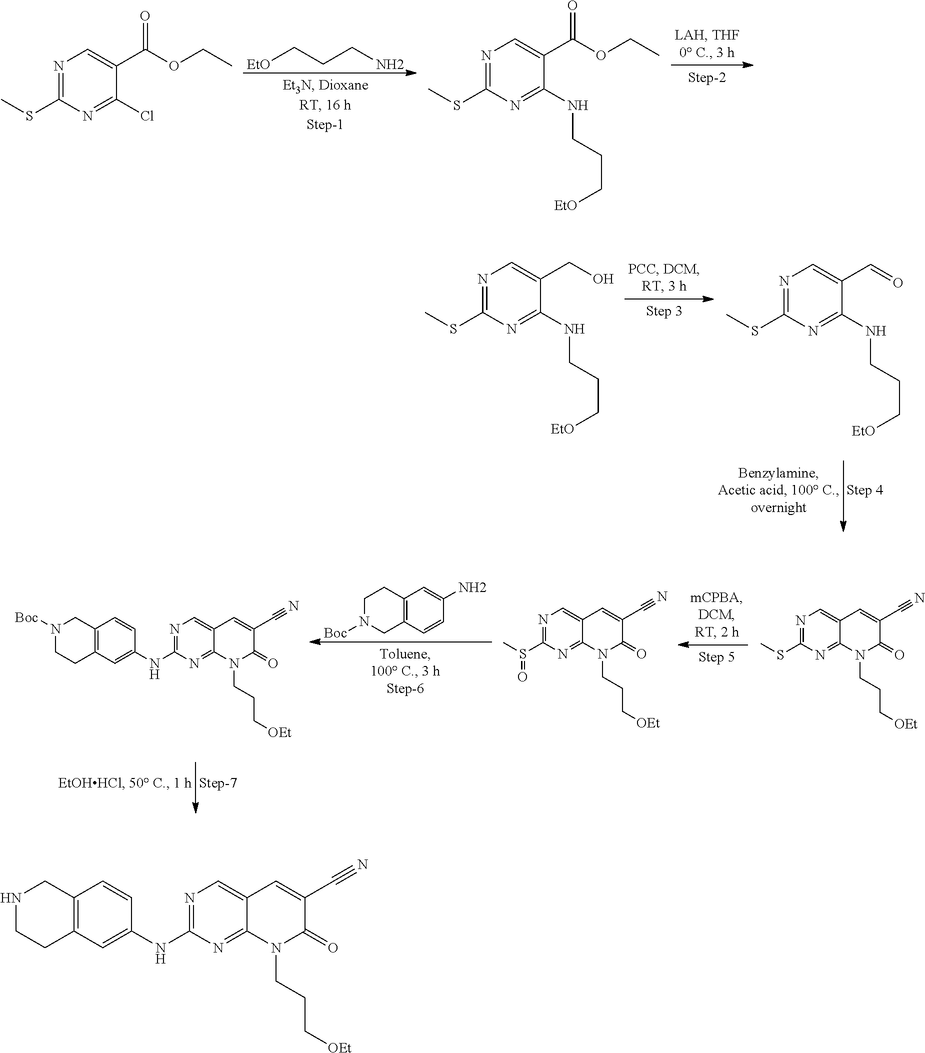

Step—1: Synthesis of ethyl 4-((3-ethoxypropyl)amino)-2-(methylthio)pyrimidine-5-carboxylate: To a stirred solution of ethyl 4-chloro-2-(methylthio)pyrimidine-5-carboxylate (3000 mg, 12.9 mmol, 1 equiv) in 1,4-Dioxane (30 mL), was added Et₃N (3.6 mL, 25.79 mmol, 2.0 equiv) and 3-ethoxypropan-1-amine (1600 mg, 15.47 mmol, 1.2 equiv) at RT. The resultant reaction mixture was allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain title compound.

Step—2: Synthesis of (4-((3-ethoxypropyl)amino)-2-(methylthio)pyrimidin-5-yl)methanol: To a stirred solution of ethyl 4-((3-ethoxypropyl)amino)-2-(methylthio)pyrimidine-5-carboxylate (3000 mg, 10.0 mmol, 1 equiv) in THF (30 mL), was added LAH (760 mg, 20.0 mmol, 2 equiv) at 0° C. The reaction mixture was allowed to stir at 0° C. for 3 h. Progress of the reaction was monitored by TLC and LCMS. After completion of the reaction, the reaction mixture was quenched with water (10 ml) followed by the addition of 10% solution of sodium hydroxide (5 mL) at 0° C. and stirred for 10 min at RT. The resulting mixture was filtered through celite and dried over anhydrous sodium sulphate and concentrated under reduced pressure to obtain the product. Organic layer was dried over anhydrous sodium sulphate and concentrated under reduced pressure to obtain title compound.

Step—3: Synthesis of 4-((3-ethoxypropyl)amino)-2-(methylthio)pyrimidine-5-carbaldehyde: To a stirred solution of (4-((3-ethoxypropyl)amino)-2-(methylthio)pyrimidin-5-yl)methanol (2000 mg, 7.78 mmol, 1 equiv) in DCM (20 mL), was added PCC (1840 mg, 8.55 mmol, 1.1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain title compound.

Step—4: Synthesis of 8-(3-ethoxypropyl)-2-(methylthio)-7-oxo-7,8-dihydropyrido[2,3-d]pyrimidine-6-carbonitrile: To a stirred solution of 4-((3-ethoxypropyl)amino)-2-(methylthio)pyrimidine-5-carbaldehyde (1000 mg, 3.92 mmol, 1.0 equiv) in Acetic acid (15 mL), was added Cyanoacetic acid (430 mg, 5.09 mmol, 1.3 equiv) and Benzyl amine (42 mg, 0.39, 0.1 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain title compound.

Step—5: Synthesis of 8-(3-ethoxypropyl)-2-(methylsulfinyl)-7-oxo-7,8-dihydropyrido[2,3-d]pyrimidine-6-carbonitrile: To a stirred solution of 8-(3-ethoxypropyl)-2-(methylthio)-7-oxo-7,8-dihydropyrido[2,3-d]pyrimidine-6-carbonitrile (200 mg, 0.66 mmol, 1.0 equiv) in DCM (6 mL), was added m-CPBA (150 mg, 0.86 mmol, 1.3 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain title compound.

Step—6: Synthesis of tert-butyl 6-((6-cyano-8-(3-ethoxypropyl)-7-oxo-7,8-dihydropyrido[2,3-d]pyrimidin-2-yl)amino)-3,4-dihydroisoquinoline-2(1H)-carboxylate: To a stirred solution of 8-(3-ethoxypropyl)-2-(methylsulfinyl)-7-oxo-7,8-dihydropyrido[2,3-d]pyrimidine-6-carbonitrile (200 mg, 0.63 mmol, 1.0 equiv) in toluene (5 mL), was added tert-butyl 6-amino-3,4-dihydroisoquinoline-2(1H)-carboxylate (160 mg, 0.63 mmol, 1.0 equiv). The resultant reaction mixture was allowed to stir at 100° C. for 3 h. Progress of the reaction was monitored by LCMS. After completion of the reaction, solid observed was filtered and dried under vacuum to obtain crude compound, which was triturated methanol to obtain title compound.

Step—7: Synthesis of 8-(3-ethoxypropyl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-(3-ethoxypropyl)-7-oxo-7,8-dihydropyrido[2,3-d]pyrimidin-2-yl)amino)-3,4-dihydroisoquinoline-2(1H)-carboxylate (100 mg, 0.20 mmol, 1 equiv) in 1.25 M HCl in ethanol (5 mL) was allowed to stir for 1 h at 50° C. Progress of the reaction was monitored by LCMS. After completion of the reaction, solvent was removed under reduced pressure to obtain residue, which was dried under lyophilizer to obtain title compound. LCMS: 405 [M+H]⁺; ¹H NMR (400 MHz, DMSO-d₆) δ ppm 8.84 (s, 1H) 8.55 (s, 1H) 7.60-7.70 (m, 2H) 7.21 (d, J=8.7 Hz, 1H) 4.32 (t, J=6.8 Hz, 2H) 4.23 (s, 2H) 3.39 (dt, J=19.7, 6.1 Hz, 4H) 3.30 (q, J=7.0 Hz, 2H) 3.01 (t, J=5.9 Hz, 2H) 1.85-1.97 (m, 2H) 0.97 (t, J=7.0 Hz, 3H).

### Example—Sill: Synthesis of 8-(3,3-difluorocyclopentyl)-7-oxo-2-((1,2,3,4-tetrahydroisoquinolin-6-yl)amino)-7,8-dihydropyrido[2,3-d]pyrimidine-6-carbonitrile (Compound No. 344)

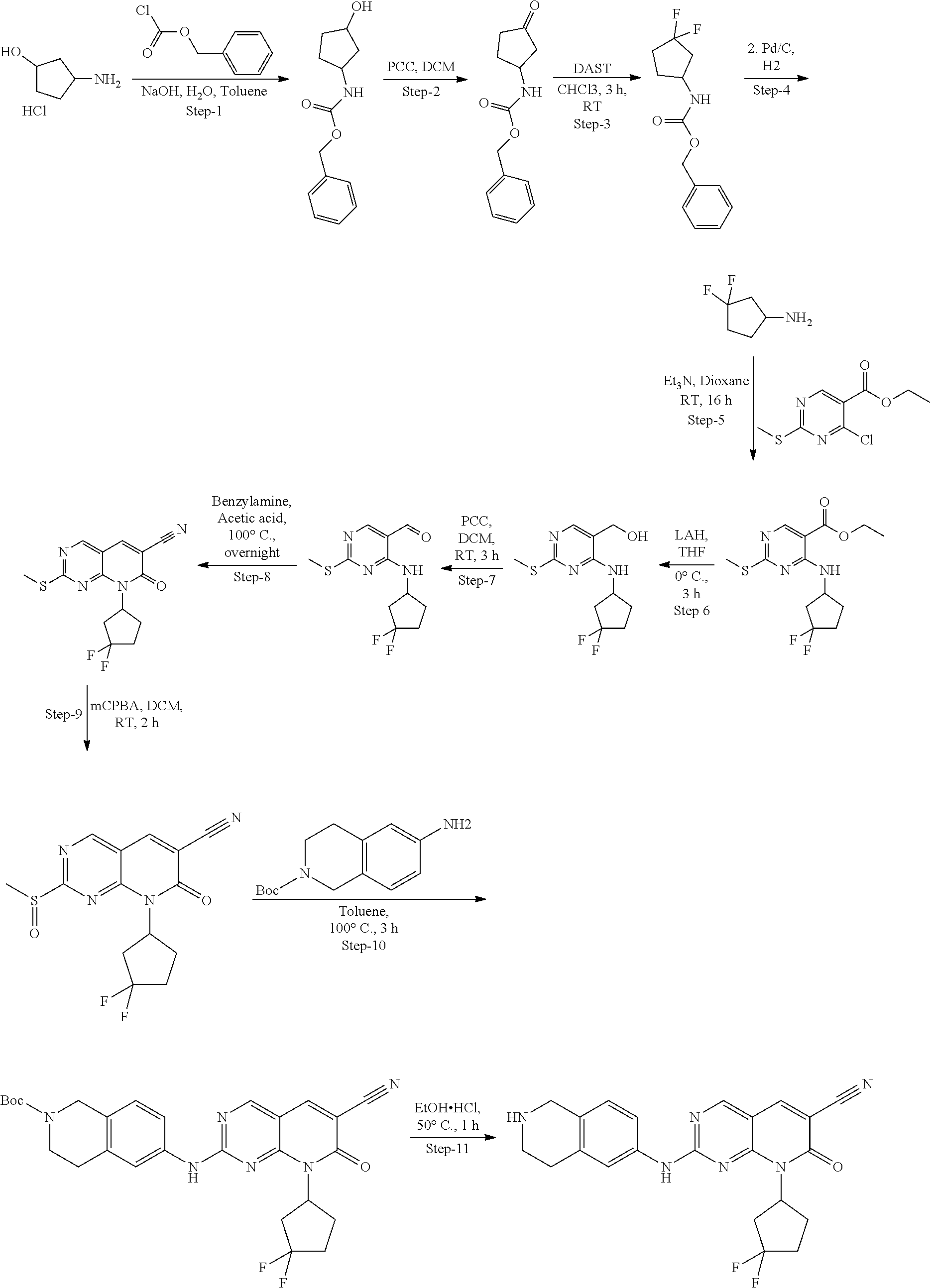

Step—1: Synthesis of benzyl (3-hydroxycyclopentyl)carbamate: To a solution of 3-aminocyclopentan-1-ol, hydrochloride salt (4 g, 29.07 mmol, 1.0 eq) in water (20 mL) at 0° C. Aqueous NaOH (3M, 21.32 mL, 2.2 equiv) and benzyl chloroformate (4.55 mL, 31.98 mmol, 1.1 equiv) were added by turns. The reaction mixture was allowed to stir at 0° C. for 3H. After completion of the reaction (monitored by LCMS), reaction mixture was extracted with DCM (3×30 mL). The combined organic layer was dried over sodium sulfate, filtered and concentrated in vacuo which was purified by normal phase combi flash to obtain the desired product.

Step—2: Synthesis of benzyl (3-oxocyclopentyl)carbamate: To a stirred solution of benzyl (3-hydroxycyclopentyl)carbamate (4.0 g, 17.0 mmol, 1 equiv) in DCM (30 mL), was added PCC (4.03 g, 18.7 mmol, 1.1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain the desired product.

Step—3: Synthesis of benzyl (3,3-difluorocyclopentyl)carbamate: To the stirred solution of benzyl (3-oxocyclopentyl)carbamate (3.0 g, 12.86 mmol, 1.0 equiv) in CHCl₃ (30 mL) at 0° C. was added DAST (8.50 mL, 64.30 mmol, 5.0 equiv) under an atmosphere of nitrogen. The resulting mixture was then allowed to warm to RT and stirred for 3H, until the completion of the reaction. Progress of the reaction was monitored by TLC and LCMS. After completion of the reaction, aq. NaHCO₃(30 mL) was added to the reaction mixture and organic layer was separated and washed with water (20 mL), brine (20 mL) and dried over sodium sulfate. Volatiles were removed under vacuum to give the desired product.

Step—4: Synthesis of 3,3-difluorocyclopentan-1-amine: To a solution of benzyl (3,3-difluorocyclopentyl)carbamate (2.5 g, 9.79 mmol, 1.0 equiv) in MeOH (20 mL) at RT under an atmosphere of nitrogen was added Pd/C (0.25 g, 10% w/w) in one portion. The resulting mixture was then stirred under a hydrogen balloon (2 Liter) overnight. The reaction mixture was filtered and the filtrate was concentrated under reduced pressure to give the desired product which was used directly in the next step without any further purification.

Step—5: Synthesis of ethyl 4-((3,3-difluorocyclopentyl)amino)-2-(methylthio)pyrimidine-5-carboxylate: To a stirred solution of ethyl 4-chloro-2-(methylthio)pyrimidine-5-carboxylate (2.0 g, 8.58 mmol, 1 equiv) in Dioxane (20 mL), was added 3,3-difluorocyclopentan-1-amine (1.09 g, 9.05 mmol, 1.05 equiv) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain the product.

Step—6: Synthesis of (4-((3,3-difluorocyclopentyl)amino)-2-(methylthio)pyrimidin-5-yl)methanol: To a stirred solution of ethyl 4-((3,3-difluorocyclopentyl)amino)-2-(methylthio)pyrimidine-5-carboxylate (2.0 g, 6.31 mmol, 1.0 equiv) in THF (20 mL), was added LAH (479 mg, 12.62 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium hydroxide (100 mL) at 0° C. and extracted with ethyl acetate (150 mL×2). Organic layer was washed with water (100 mL) and brine solution (100 mL). Organic layer was dried over anhydrous sodium sulphate and concentrated under reduced pressure to obtain the product.

Step—7: Synthesis of 4-((3,3-difluorocyclopentyl)amino)-2-(methylthio)pyrimidine-5-carbaldehyde: To a stirred solution of 4-((3,3-difluorocyclopentyl)amino)-2-(methylthio)pyrimidine-5-carbaldehyde (1.4 g, 5.09 mmol, 1 equiv) in DCM (20 mL), was added PCC (1.21 g, 5.60 mmol, 1.1 equiv) at RT. The reaction mixture was then allowed to stir at RT for 3 h. Progress of the reaction was monitored by TLC and LCMS. After completion of the reaction, the reaction mixture was passes through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain the product.

Step—8: Synthesis of 8-(3,3-difluorocyclopentyl)-2-(methylthio)-7-oxo-7,8-dihydropyrido[2,3-d]pyrimidine-6-carbonitrile: To a stirred solution of 4-((3,3-difluorocyclopentyl)amino)-2-(methylthio)pyrimidine-5-carbaldehyde (1.0 g, 3.66 mmol, 1.0 equiv) in Acetic acid (10 mL), was added Cyanoacetic acid (374 mg, 4.40 mmol, 1.2 equiv) and Benzyl amine (0.04 mL, 0.37, 0.1 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain the product.

Step—9: Synthesis of 8-(3,3-difluorocyclopentyl)-2-(methylsulfinyl)-7-oxo-7,8-dihydropyrido[2,3-d]pyrimidine-6-carbonitrile: To a stirred solution of 8-(3,3-difluorocyclopentyl)-2-(methylthio)-7-oxo-7,8-dihydropyrido[2,3-d]pyrimidine-6-carbonitrile (200 mg, 0.62 mmol, 1 equiv) in DCM (5 mL), was added m-CPBA (0.15 g, 0.87 mmol, 1.4 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain the product.

Step—10: Synthesis of tert-butyl 6-((6-cyano-8-(3,3-difluorocyclopentyl)-7-oxo-7,8-dihydropyrido[2,3-d]pyrimidin-2-yl)amino)-3,4-dihydroisoquinoline-2(1H)-carboxylate: To a stirred solution of 8-(3,3-difluorocyclopentyl)-2-(methylsulfinyl)-7-oxo-7,8-dihydropyrido[2,3-d]pyrimidine-6-carbonitrile (100 mg, 0.30 mmol, 1 equiv) in toluene (5 mL), was added tert-butyl 6-amino-3,4-dihydroisoquinoline-2(1H)-carboxylate (73 mg, 0.30 mmol, 1.0 equiv). The resultant reaction mixture was allowed to stir at 100° C. for 3 h. Progress of the reaction was monitored by LCMS. After completion of the reaction, solid observed was filtered and dried under vacuum to obtain crude compound, which was purified by trituration with methanol to obtain the desired product.

Step—11: Synthesis of 8-(3,3-difluorocyclopentyl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-(3,3-difluorocyclopentyl)-7-oxo-7,8-dihydropyrido[2,3-d]pyrimidin-2-yl)amino)-3,4-dihydroisoquinoline-2(1H)-carboxylate (80 mg, 0.15 mmol, 1 equiv) in 1.25 M HCl in ethanol (5 mL) was allowed to stir for 1 h at 50° C. Progress of the reaction was monitored by LCMS. After completion of the reaction, the resultant precipitates were filtered and washed with ethanol to obtain the desired product. LCMS: 423 [M+H]⁺; ¹H NMR (400 MHz, DMSO-d₆, D₂O) δ ppm 8.81-8.93 (m, 1H) 8.58 (s, 1H) 7.67 (br s, 1H) 7.47 (br s, 1H) 7.21 (d, J=8.3 Hz, 1H) 6.04 (br s, 1H) 4.23 (s, 2H) 3.37 (t, J=6.1 Hz, 2H) 2.84-3.05 (m, 3H) 2.43 (br s, 1H) 2.18-2.39 (m, 2H) 2.09 (br s, 2H)

### Example—S112: Synthesis of 8-(3,3-difluorocyclopentyl)-2-((1-(methylsulfonyl)piperidin-4-yl)amino)-7-oxo-7,8-dihydropyrido[2,3-d]pyrimidine-6-carbonitrile (Compound No. 345), (S)-8-(3,3-difluorocyclopentyl)-2-(1-(methylsulfonyl)piperidin-4-ylamino)-7-oxo-7,8-dihydropyrido[2,3-d]pyrimidine-6-carbonitrile (Compound No. 384) and (R)-8-(3,3-difluorocyclopentyl)-2-(1-(methylsulfonyl)piperidin-4-ylamino)-7-oxo-7,8-dihydropyrido[2,3-d]pyrimidine-6-carbonitrile (Compound No. 385)

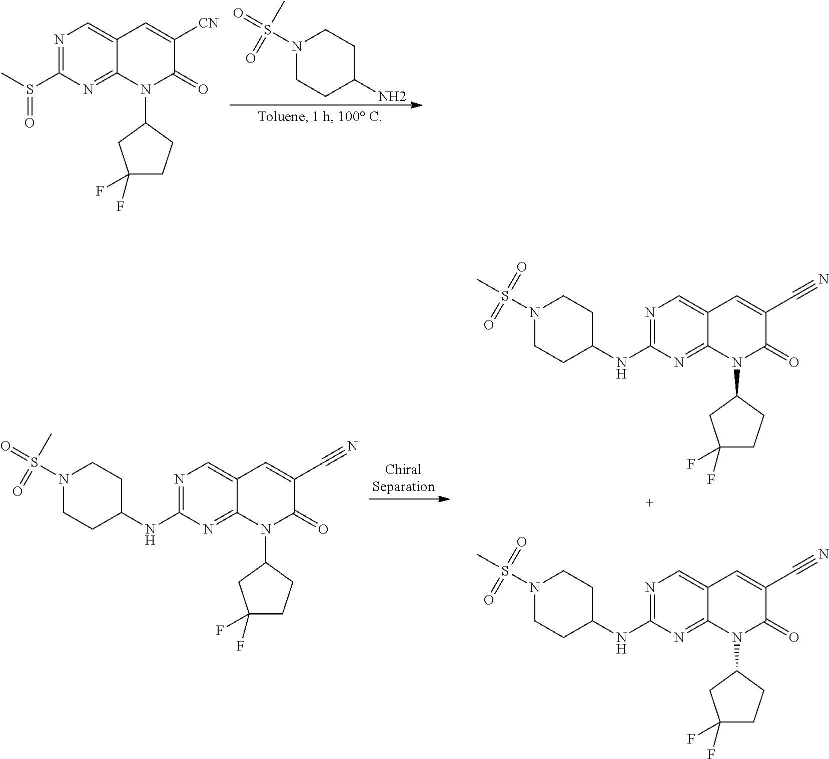

To the stirred solution of 1-(isopropylsulfonyl)piperidin-4-amine (0.1 g, 0.56 mmol, 1.0 equiv) in Toluene (5 mL) 8-(3,3-difluorocyclopentyl)-2-(methylsulfinyl)-7-oxo-7,8-dihydropyrido[2,3-d]pyrimidine-6-carbonitrile (0.19 g, 0.56 moles, 1.0 equiv). The reaction mixture was then stirred at 100° C. for 1H. After completion of the reaction, toluene was evaporated under vacuum and product was purified using reverse phase HPLC. Racemate was purified by chiral chromatography to give the desired products. LCMS: 453 [M+H]⁺; ¹HNMR (400 MHz, DMSO-d₆, D₂O) δ ppm 8.69 (br s, 1H) 8.40 (s, 1H) 5.98 (br s, 1H) 3.98 (br s, 1H) 3.60 (d, J=12.7 Hz, 2H) 3.03 (br s, 1H) 2.82-2.93 (m, 4H) 2.54-2.72 (m, 2H) 2.33 (br s, 1H) 2.28 (br s, 1H) 2.15 (br s, 1H) 2.06 (br s, 1H) 1.97 (br s, 2H) 1.64 (d, J=11.4 Hz, 2H)

### Example—S113: Synthesis of 8-(4-hydroxytetrahydrofuran-3-yl)-7-oxo-2-((1,2,3,4-tetrahydroisoquinolin-6-yl)amino)-7,8-dihydropyrido[2,3-d]pyrimidine-6-carbonitrile (Compound No. 101)

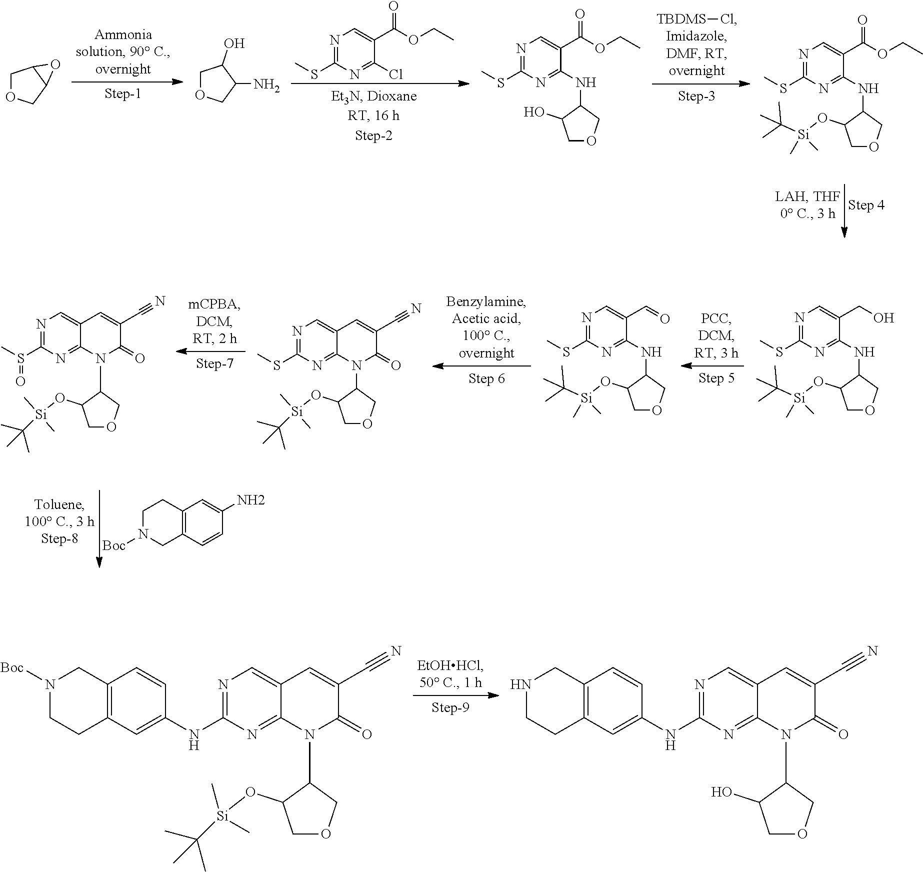

Step—1: Synthesis of 4-aminotetrahydrofuran-3-ol: A solution of tert-butyl 4-(4-((6-cyano-8-cyclopentyl-7-oxo-7,8-dihydropyrido[2,3-d]pyrimidin-2-yl)amino)-3-methyl-1H-pyrazol-1-yl)piperidine-1-carboxylate (2000 mg, 23.8 mmol, 1 equiv) in Ammonia solution (15 mL) was allowed to stir at 90° C. for overnight in a sealed tube. Progress of the reaction was monitored by TLC. After completion of the reaction, solvent was removed under reduced pressure and the residue was dried under Lyophiliser to obtain crude, which was used for the next step without any further purification.

Step—2: Synthesis of ethyl 4-((4-hydroxytetrahydrofuran-3-yl)amino)-2-(methylthio)pyrimidine-5-carboxylate: To a stirred solution of ethyl 4-chloro-2-(methylthio)pyrimidine-5-carboxylate (2000 mg, 8.62 mmol, 1 equiv) in Dioxane (20 mL), was added ET₃N (2.4 mL, 17.24 mmol, 2 equiv) and 4-aminotetrahydrofuran-3-ol (1066 mg, 10.34 mmol, 1.2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain crude, which was used for the next step without any further purification.

Step—3: Synthesis of ethyl 4-((4-((tert-butyldimethylsilyl)oxy)tetrahydrofuran-3-yl)amino)-2-(methylthio)pyrimidine-5-carboxylate: To a stirred solution of ethyl 4-((4-hydroxytetrahydrofuran-3-yl)amino)-2-(methylthio)pyrimidine-5-carboxylate (1000 mg, 3.34 mmol, 1 equiv) in DMF (20 mL), was added Imidazole (682 mg, 6.68 mmol, 3 equiv) and TBDMSCl (1009 mg, 10.03 mmol, 2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain crude, which was used for the next step without any further purification.

Step—4: Synthesis of (4-((4-((tert-butyldimethylsilyl)oxy)tetrahydrofuran-3-yl)amino)-2-(methylthio)pyrimidin-5-yl)methanol: To a stirred solution of ethyl 4-((4-((tert-butyldimethylsilyl)oxy)tetrahydrofuran-3-yl)amino)-2-(methylthio)pyrimidine-5-carboxylate (1400 mg, 3.38 mmol, 1 equiv) in THF (20 mL), was added LAH (258 mg, 6.77 mmol, 2 equiv) at 0° C. Raise the temp. To RT and the reaction mixture was allowed to stir for 1 h. Progress of the reaction was monitored by TLC and LCMS. After completion of the reaction, the reaction mixture was quenched with saturated solution of sodium hydroxide (5 mL) at 0° C., the reaction mixture was then passes through celite bed, filtrate obtain was concentrated under reduced pressure to obtain crude, which was used for the next step without any further purification.

Step—5: Synthesis of 4-((4-((tert-butyldimethylsilyl)oxy)tetrahydrofuran-3-yl)amino)-2-(methylthio)pyrimidine-5-carbaldehyde: To a stirred solution of (4-((4-((tert-butyldimethylsilyl)oxy)tetrahydrofuran-3-yl)amino)-2-(methylthio)pyrimidin-5-yl)methanol (1100 mg, 2.96 mmol, 1 equiv) in DCM (15 mL), was added PCC (640 mg, 2.96 mmol, 1 equiv) at RT. The reaction mixture was allowed to stir at RT for 1 h. Progress of the reaction was monitored by TLC and LCMS. After completion of the reaction, the reaction mixture was passes through celite bed; filtrate was concentrated under reduced pressure to obtain crude, which was used for the next step without any further purification.

Step—6: Synthesis of 8-(4-((tert-butyldimethylsilyl)oxy)tetrahydrofuran-3-yl)-2-(methylthio)-7-oxo-7,8-dihydropyrido[2,3-d]pyrimidine-6-carbonitrile: To a stirred solution of 4-((4-((tert-butyldimethylsilyl)oxy)tetrahydrofuran-3-yl)amino)-2-(methylthio)pyrimidine-5-carbaldehyde (1000 mg, 2.71 mmol, 1 equiv) in Acetic acid (10 mL), was added Cyanoacetic acid (276 mg, 3.25 mmol, 1.2 equiv) and Benzyl amine (0.03 mL, 0.27 mmol, 0.1 equiv). The reaction mixture was allowed to stir at 100° C. for overnight. Progress of the reaction was monitored by TLC and LCMS. After completion of the reaction, the reaction mixture was diluted with water (100 mL) and extracted with ethyl acetate (150 mL). Organic layer was washed with water (100 mL), brine solution (100 mL). Organic layer was dried over anhydrous sodium sulphate and concentrated under reduced pressure to obtain crude, which was used for the next step without any further purification.

Step—7: Synthesis of 8-(4-((tert-butyldimethylsilyl)oxy)tetrahydrofuran-3-yl)-2-(methylsulfinyl)-7-oxo-7,8-dihydropyrido[2,3-d]pyrimidine-6-carbonitrile: To a stirred solution of 8-(4-((tert-butyldimethylsilyl)oxy)tetrahydrofuran-3-yl)-2-(methylthio)-7-oxo-7,8-dihydropyrido[2,3-d]pyrimidine-6-carbonitrile (200 mg, 0.47 mmol, 1 equiv) in DCM (10 mL), was added m-CPBA (106 mg, 0.61 mmol, 1.3 equiv) at RT. Then the reaction mixture was allowed to stir for 1 h. Progress of the reaction was monitored by LCMS. After completion of the reaction, the reaction mixture was diluted with DCM (100 mL) and washed with water (50 mL), brine solution (50 mL). Organic layer was dried over anhydrous sodium sulphate and concentrated under reduced pressure to obtain crude, which was used for the next step without any further purification.

Step—8: Synthesis of tert-butyl 6-((8-(4-((tert-butyldimethylsilyl)oxy)tetrahydrofuran-3-yl)-6-cyano-7-oxo-7,8-dihydropyrido[2,3-d]pyrimidin-2-yl)amino)-3,4-dihydroisoquinoline-2(1H)-carboxylate: To a stirred solution of 8-(4-((tert-butyldimethylsilyl)oxy)tetrahydrofuran-3-yl)-2-(methylsulfinyl)-7-oxo-7,8-dihydropyrido[2,3-d]pyrimidine-6-carbonitrile (100 mg, 0.23 mmol, 1 equiv) in toluene (5 mL), was added tert-butyl 6-amino-3,4-dihydroisoquinoline-2(1H)-carboxylate (63 mg, 0.25 mmol, 1.1 equiv). The resultant reaction mixture was stir at 100° C. for 2 h. Progress of the reaction was monitored by LCMS. After completion of the reaction, solvent was removed under reduced pressure to obtain crude, which was used for the next step without any further purification.

Step—9: Synthesis of 8-(4-hydroxytetrahydrofuran-3-yl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((8-(4-((tert-butyldimethylsilyl)oxy)tetrahydrofuran-3-yl)-6-cyano-7-oxo-7,8-dihydropyrido[2,3-d]pyrimidin-2-yl)amino)-3,4-dihydroisoquinoline-2(1H)-carboxylate (140 mg, 0.22 mmol, 1 equiv) in 1.25 M HCl in ethanol (5 mL) was allowed to stir at 50° C. for overnight. Progress of the reaction was monitored by LCMS. After completion of the reaction, solvent was removed under reduced pressure and the residue was dried under lyophilizer to obtain crude compound, which was purified by reverse phase HPLC. LCMS: 405 [M+H]⁺, ¹H NMR (DMSO-d₆, 400 MHz): δ ppm 10.67 (br s, 1H), 8.99 (br s, 2H), 8.89 (s, 1H), 8.65 (s, 1H), 7.76 (br s, 1H), 7.49 (br s, 1H), 7.22 (d, J=8.3 Hz, 1H), 5.86 (br s, 1H), 5.30 (br s, 1H), 4.88 (br s, 1H), 4.26 (br s, 2H), 4.14 (br s, 1H), 4.00 (t, J=8.8 Hz, 1H), 3.89 (t, J=8.3 Hz, 1H), 3.67 (d, J=8.3 Hz, 1H), 3.41 (s, 2H), 2.88-3.14 (m, 2H). ¹H NMR (DMSO-d₆+D₂O, 400 MHz): δ ppm 8.89 (s, 1H), 8.65 (s, 1H), 7.76 (br s, 1H), 7.49 (br s, 1H), 7.22 (d, J=8.3 Hz, 1H), 5.86 (br s, 1H), 4.88 (br s, 1H), 4.26 (br s, 2H), 4.14 (br s, 1H), 4.00 (t, J=8.8 Hz, 1H), 3.89 (t, J=8.3 Hz, 1H), 3.67 (d, J=8.3 Hz, 1H), 3.41 (s, 2H), 2.88-3.14 (m, 2H).

### Example—S114: Synthesis of 8-(2-hydroxycyclopentyl)-7-oxo-2-((1,2,3,4-tetrahydroisoquinolin-6-yl)amino)-7,8-dihydropyrido[2,3-d]pyrimidine-6-carbonitrile (Compound No. 346)

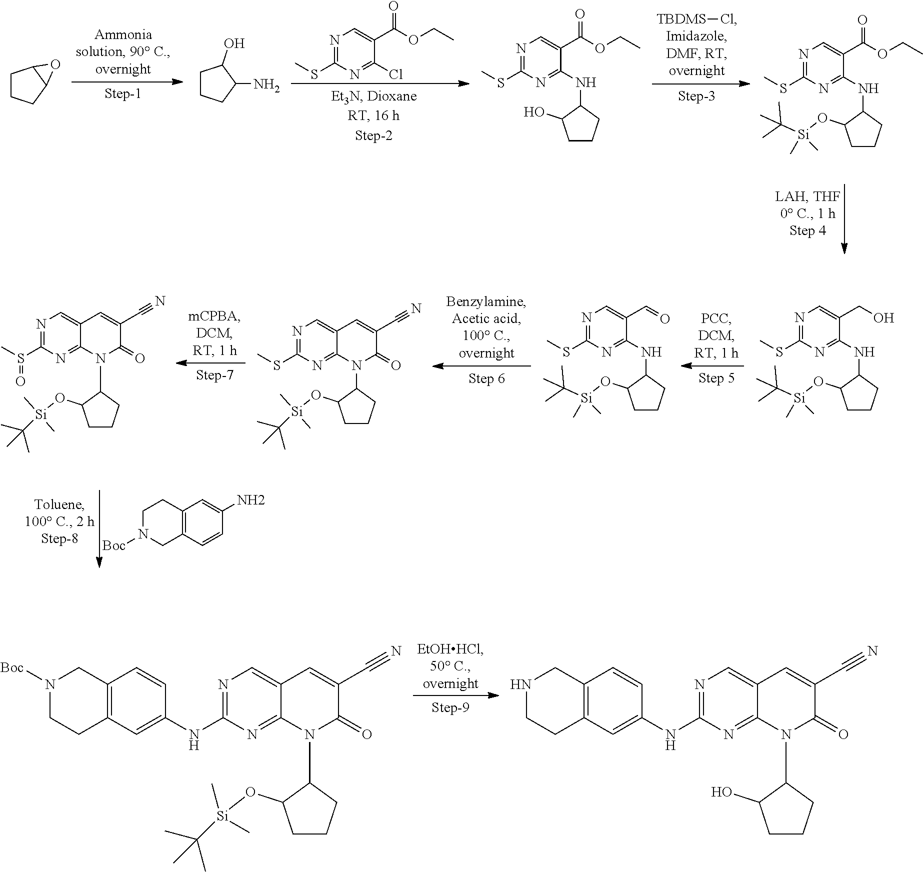

Step—1: Synthesis of 2-aminocyclopentan-1-ol: A solution of tert-butyl 4-(4-((6-cyano-8-cyclopentyl-7-oxo-7,8-dihydropyrido[2,3-d]pyrimidin-2-yl)amino)-3-methyl-1H-pyrazol-1-yl)piperidine-1-carboxylate (2000 mg, 23.8 mmol, 1 equiv) in Ammonia solution (15 mL) was allowed to stir at 90° C. for overnight in a sealed tube. Progress of the reaction was monitored by TLC. After completion of the reaction, solvent was removed under reduced pressure and the residue was dried under Lyophiliser to obtain crude, which was used for the next step without any further purification.

Step—2: Synthesis of ethyl 4-((2-hydroxycyclopentyl)amino)-2-(methylthio)pyrimidine-5-carboxylate: To a stirred solution of ethyl 4-chloro-2-(methylthio)pyrimidine-5-carboxylate (2000 mg, 8.62 mmol, 1 equiv) in Dioxane (20 mL), was added ET₃N (2.4 mL, 17.24 mmol, 2 equiv) and 2-aminocyclopentan-1-ol (1066 mg, 10.34 mmol, 1.2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain crude, which was used for the next step without any further purification.

Step—3: Synthesis of ethyl 4-((2-((tert-butyldimethylsilyl)oxy)cyclopentyl)amino)-2-(methylthio)pyrimidine-5-carboxylate: To a stirred solution of ethyl 4-((2-hydroxycyclopentyl)amino)-2-(methylthio)pyrimidine-5-carboxylate (2000 mg, 6.73 mmol, 1 equiv) in DMF (20 mL), was added Imidazole (1373 mg, 20.19 mmol, 3 equiv) and TBDMSCl (2033 mg, 13.46 mmol, 2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain crude, which was used for the next step without any further purification

Step—4: Synthesis of (4-((2-((tert-butyldimethylsilyl)oxy)cyclopentyl)amino)-2-(methylthio)pyrimidin-5-yl)methanol: To a stirred solution of ethyl 4-((2-((tert-butyldimethylsilyl)oxy)cyclopentyl)amino)-2-(methylthio)pyrimidine-5-carboxylate (2500 mg, 6.08 mmol, 1 equiv) in THF (30 mL), was added LAH (462 mg, 12.16 mmol, 2 equiv) at 0° C. Raise the temp. to RT and the reaction mixture was allowed to stir for 1 h. Progress of the reaction was monitored by TLC and LCMS. After completion of the reaction, the reaction mixture was quenched with saturated solution of sodium hydroxide (5 mL) at 0° C., the reaction mixture was then passes through celite bed, filtrate obtain was concentrated under reduced pressure to obtain crude, which was used for the next step without any further purification.

Step—5: Synthesis of 4-((2-((tert-butyldimethylsilyl)oxy)cyclopentyl)amino)-2-(methylthio)pyrimidine-5-carbaldehyde: To a stirred solution of (4-((2-((tert-butyldimethylsilyl)oxy)cyclopentyl)amino)-2-(methylthio)pyrimidin-5-yl)methanol (1500 mg, 4.06 mmol, 1 equiv) in DCM (20 mL), was added PCC (878 mg, 4.06 mmol, 1 equiv) at RT. The reaction mixture was allowed to stir at RT for 1 h. Progress of the reaction was monitored by TLC and LCMS. After completion of the reaction, the reaction mixture was passes through celite bed; filtrate was concentrated under reduced pressure to obtain crude, which was used for the next step without any further purification.

Step—6: Synthesis of 8-(2-((tert-butyldimethylsilyl)oxy)cyclopentyl)-2-(methylthio)-7-oxo-7,8-dihydropyrido[2,3-d]pyrimidine-6-carbonitrile: To a stirred solution of 4-((2-((tert-butyldimethylsilyl)oxy)cyclopentyl)amino)-2-(methylthio)pyrimidine-5-carbaldehyde (1500 mg, 4.08 mmol, 1 equiv) in Acetic acid (10 mL), was added Cyanoacetic acid (417 mg, 4.9 mmol, 1.2 equiv) and Benzyl amine (0.04 mL, 0.4 mmol, 0.1 equiv). The reaction mixture was allowed to stir at 100° C. for overnight. Progress of the reaction was monitored by TLC and LCMS. After completion of the reaction, the reaction mixture was diluted with water (100 mL) and extracted with ethyl acetate (150 mL). Organic layer was washed with water (100 mL), brine solution (100 mL). Organic layer was dried over anhydrous sodium sulphate and concentrated under reduced pressure to obtain crude, which was used for the next step without any further purification.

Step—7: Synthesis of 8-(2-((tert-butyldimethylsilyl)oxy)cyclopentyl)-2-(methylsulfinyl)-7-oxo-7,8-dihydropyrido[2,3-d]pyrimidine-6-carbonitrile: To a stirred solution of 8-(2-((tert-butyldimethylsilyl)oxy)cyclopentyl)-2-(methylthio)-7-oxo-7,8-dihydropyrido[2,3-d]pyrimidine-6-carbonitrile (1000 mg, 2.4 mmol, 1 equiv) in DCM (10 mL), was added m-CPBA (547 mg, 3.12 mmol, 1.3 equiv) at RT. Then the reaction mixture was allowed to stir for 1 h. Progress of the reaction was monitored by LCMS. After completion of the reaction, the reaction mixture was diluted with DCM (100 mL) and washed with water (50 mL), brine solution (50 mL). Organic layer was dried over anhydrous sodium sulphate and concentrated under reduced pressure to obtain crude, which was used for the next step without any further purification.

Step—8: Synthesis of tert-butyl 6-((8-(2-((tert-butyldimethylsilyl)oxy)cyclopentyl)-6-cyano-7-oxo-7,8-dihydropyrido[2,3-d]pyrimidin-2-yl)amino)-3,4-dihydroisoquinoline-2(1H)-carboxylate: To a stirred solution of 8-(2-((tert-butyldimethylsilyl)oxy)cyclopentyl)-2-(methylsulfinyl)-7-oxo-7,8-dihydropyrido[2,3-d]pyrimidine-6-carbonitrile (200 mg, 0.46 mmol, 1 equiv) in toluene (5 mL), was added tert-butyl 6-amino-3,4-dihydroisoquinoline-2(1H)-carboxylate (126 mg, 0.50 mmol, 1.1 equiv). The resultant reaction mixture was stir at 100° C. for 2 h. Progress of the reaction was monitored by LCMS. After completion of the reaction, solvent was removed under reduced pressure to obtain crude, which was used for the next step without any further purification.

Step—9: Synthesis of 8-(2-hydroxycyclopentyl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((8-(2-((tert-butyldimethylsilyl)oxy)cyclopentyl)-6-cyano-7-oxo-7,8-dihydropyrido[2,3-d]pyrimidin-2-yl)amino)-3,4-dihydroisoquinoline-2(1H)-carboxylate (300 mg, 0.48 mmol, 1 equiv) in 1.25 M HCl in ethanol (5 mL) was allowed to stir at 50° C. for overnight. Progress of the reaction was monitored by LCMS. After completion of the reaction, solvent was removed under reduced pressure and the residue was dried under Lyophiliser to obtain crude compound, which was purified by reverse phase HPLC. LCMS: 403 [M+H]⁺, ¹H NMR (400 MHz, DMSO-d₆) δ ppm 10.63 (br s, 1H), 8.99 (br s, 2H), 8.88 (s, 1H) 8.62 (s, 1H), 7.85 (br s, 1H), 7.49 (br s, 1H), 7.22 (d, J=8.3 Hz, 1H), 5.63 (br s, 1H), 4.89 (br s, 1H), 4.77 (d, J=6.1 Hz, 1H), 4.26 (br s, 2H), 3.33 (m, 2H), 3.02 (d, J=6.1 Hz, 2H), 2.13 (d, J=9.6 Hz, 2H), 1.86 (br s, 2H), 1.73 (br s, 1H), 1.55 (br s, 1H). ¹H NMR (400 MHz, DMSO-d₆+D₂O) δ ppm 8.88 (s, 1H), 8.62 (s, 1H), 7.85 (br s, 1H), 7.49 (br s, 1H), 7.22 (d, J=8.3 Hz, 1H), 5.63 (br s, 1H), 4.77 (d, J=6.1 Hz, 1H), 4.26 (br s, 2H), 3.33 (m, 2H), 3.02 (d, J=6.1 Hz, 2H), 2.13 (d, J=9.6 Hz, 2H), 1.86 (br s, 2H), 1.73 (br s, 1H), 1.55 (br s, 1H).

### Example—S115: Synthesis of 8-(cyclohexylmethyl)-7-oxo-2-((1,2,3,4-tetrahydroisoquinolin-6-yl)amino)-7,8-dihydropyrido[2,3-d]pyrimidine-6-carbonitrile (Compound No. 347)

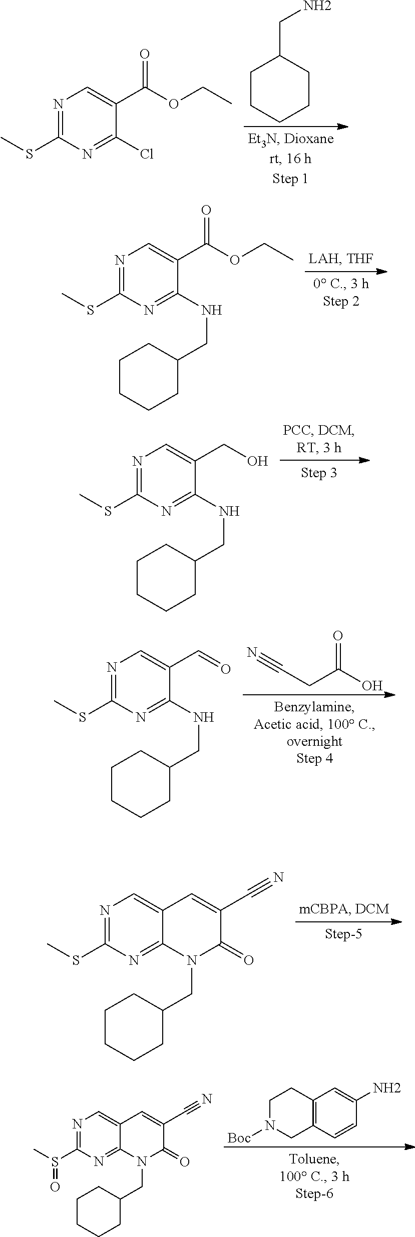

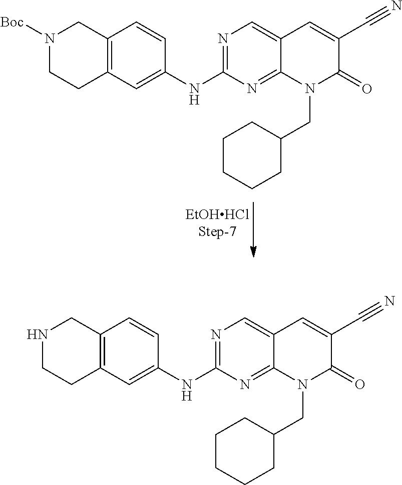 [SMILES: CC(C)(C)OC(=O)N1CCc2cc(Nc3ncc4cc(C#N)c(=O)n(CC5CCCCC5)c4n3)ccc2C1 ; N#Cc1cc2cnc(Nc3ccc4c(c3)CCNC4)nc2n(CC2CCCCC2)c1=O]

Step—1: Synthesis of ethyl 4-((cyclohexylmethyl)amino)-2-(methylthio)pyrimidine-5-carboxylate: To a stirred solution of ethyl 4-chloro-2-(methylthio)pyrimidine-5-carboxylate (3000 mg, 12.9 mmol, 1 equiv) in Dioxane (30 mL), was added ET₃N (3.6 mL, 25.8 mmol, 2 equiv) and cyclohexylmethanamine (1752 mg, 15.5 mmol, 1.2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain crude, which was used for the next step without any further purification.

Step—2: Synthesis of (4-((cyclohexylmethyl)amino)-2-(methylthio)pyrimidin-5-yl)methanol: To a stirred solution of ethyl 4-((cyclohexylmethyl)amino)-2-(methylthio)pyrimidine-5-carboxylate (2000 mg, 6.47 mmol, 1 equiv) in THF (20 mL), was added LAH (492 mg, 12.94 mmol, 2 equiv) at 0° C. Reaction mixture was allowed to stir for 1 h at RT. Progress of the reaction was monitored by TLC and LCMS. After completion of the reaction, the reaction mixture was quenched with saturated solution of sodium hydroxide (5 mL) at 0° C., the reaction mixture was then passes through celite bed, filtrate obtain was concentrated under reduced pressure to obtain crude, which was used for the next step without any further purification.

Step—3: Synthesis of 4-((cyclohexylmethyl)amino)-2-(methylthio)pyrimidine-5-carbaldehyde: To a stirred solution of (4-((cyclohexylmethyl) amino)-2-(methylthio) pyrimidin-5-yl) methanol (1000 mg, 3.74 mmol, 1 equiv) in DCM (15 mL), was added PCC (809 mg, 3.74 mmol, 1 equiv) at RT. The reaction mixture was allowed to stir at RT for 1 h. Progress of the reaction was monitored by TLC and LCMS. After completion of the reaction, the reaction mixture was passes through celite bed; filtrate was concentrated under reduced pressure to obtain crude, which was used for the next step without any further purification.

Step—4: Synthesis of 8-(cyclohexylmethyl)-2-(methylthio)-7-oxo-7,8-dihydropyrido[2,3-d]pyrimidine-6-carbonitrile: To a stirred solution of 4-((cyclohexylmethyl)amino)-2-(methylthio)pyrimidine-5-carbaldehyde (1000 mg, 3.77 mmol, 1 equiv) in Acetic acid (10 mL), was added Cyanoacetic acid (385 mg, 4.5 mmol, 1.2 equiv) and Benzyl amine (0.04 mL, 0.37 mmol, 0.1 equiv). The reaction mixture was allowed to stir at 100° C. for overnight. Progress of the reaction was monitored by TLC and LCMS. After completion of the reaction, the reaction mixture was diluted with water (100 mL) and extracted with ethyl acetate (150 mL). Organic layer was washed with water (100 mL), brine solution (100 mL). Organic layer was dried over anhydrous sodium sulphate and concentrated under reduced pressure to obtain crude, which was used for the next step without any further purification.

Step—5: Synthesis of 8-(cyclohexylmethyl)-2-(methylsulfinyl)-7-oxo-7,8-dihydropyrido[2,3-d]pyrimidine-6-carbonitrile: To a stirred solution of 8-(cyclohexylmethyl)-2-(methylthio)-7-oxo-7,8-dihydropyrido[2,3-d]pyrimidine-6-carbonitrile (200 mg, 0.64 mmol, 1 equiv) in DCM (10 mL), was added m-CPBA (154 mg, 0.89 mmol, 1.4 equiv) at RT. The reaction mixture was allowed to stir for 1 h. Progress of the reaction was monitored by LCMS. After completion of the reaction, the reaction mixture was diluted with DCM (100 mL) and washed with water (50 mL), brine solution (50 mL). Organic layer was dried over anhydrous sodium sulphate and concentrated under reduced pressure to obtain crude, which was used for the next step without any further purification.

Step—6: Synthesis of tert-butyl 6-((6-cyano-8-(cyclohexylmethyl)-7-oxo-7,8-dihydropyrido[2,3-d]pyrimidin-2-yl)amino)-3,4-dihydroisoquinoline-2(1H)-carboxylate: To a stirred solution of 8-(cyclohexylmethyl)-2-(methylsulfinyl)-7-oxo-7,8-dihydropyrido[2,3-d]pyrimidine-6-carbonitrile (80 mg, 0.24 mmol, 1 equiv) in toluene (5 mL), was added tert-butyl 6-amino-3,4-dihydroisoquinoline-2(1H)-carboxylate (66 mg, 0.26 mmol, 1.1 equiv). The resultant reaction mixture was stir at 100° C. for 2 h. Progress of the reaction was monitored by LCMS. After completion of the reaction, solvent was removed under reduced pressure to obtain crude, which was used for the next step without any further purification.

Step—7: Synthesis of 8-(cyclohexylmethyl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-(cyclohexylmethyl)-7-oxo-7,8-dihydropyrido[2,3-d]pyrimidin-2-yl)amino)-3,4-dihydroisoquinoline-2(1H)-carboxylate (100 mg, 0.19 mmol, 1 equiv) in 1.25 M HCl in ethanol (5 mL) was allowed to stir at 50° C. for overnight. Progress of the reaction was monitored by LCMS. After completion of the reaction, solvent was removed under reduced pressure and the residue was dried under Lyophiliser to obtain crude compound, which was purified by trituration with methanol. LCMS: 415 [M+H]⁺, ¹H NMR (DMSO-d₆, 400 MHz): δ ppm 10.68 (br s, 1H), 9.40 (br s, 2H), 8.87 (s, 1H), 8.64 (s, 1H), 7.70 (br s, 1H), 7.65 (s, 1H), 7.22 (d, J=8.3 Hz, 1H), 4.24 (br s, 2H), 4.15 (d, J=7.5 Hz, 2H), 3.37 (br s, 2H), 2.90-3.13 (m, 2H), 1.93 (br s, 1H), 1.67-1.57 (m, 5H), 0.98-1.26 ppm (m, 4H).

### Example—S116: Synthesis of 8-(cyclohexylmethyl)-2-((1-(methylsulfonyl)piperidin-4-yl)amino)-7-oxo-7,8-dihydropyrido[2,3-d]pyrimidine-6-carbonitrile (Compound No. 348)

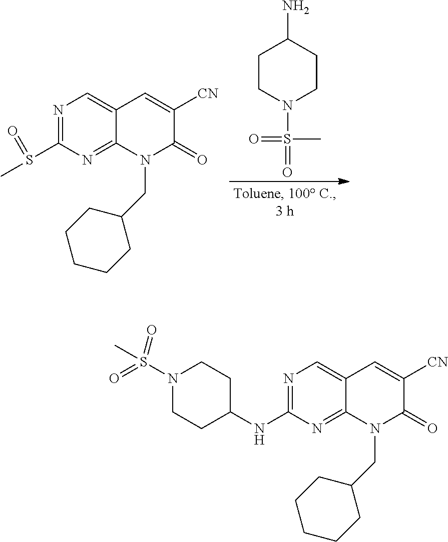 [SMILES: CS(=O)c1ncc2cc(C#N)c(=O)n(CC3CCCCC3)c2n1 ; CS(=O)(=O)N1CCC(N)CC1 ; CS(=O)(=O)N1CCC(Nc2ncc3cc(C#N)c(=O)n(CC4CCCCC4)c3n2)CC1]

To a stirred solution of 8-(cyclohexylmethyl)-2-(methylsulfinyl)-7-oxo-7,8-dihydropyrido[2,3-d]pyrimidine-6-carbonitrile (80 mg, 0.24 mmol, 1 equiv) in toluene (5 mL), was added 1-(methylsulfonyl)piperidin-4-amine (46 mg, 0.26 mmol, 1.1 equiv). The resultant reaction mixture was stir at 100° C. for 3 h. Progress of the reaction was monitored by LCMS. After completion of the reaction, solvent was removed under reduced pressure to obtain crude, which was purified by reverse phase HPLC. LCMS: 445 [M+H]⁺, ¹H NMR (DMSO-d₆, 400 MHz): δ ppm 8.69 (br s, 1H), 8.46 (s, 1H), 8.39 (br s, 1H), 4.12 (d, J=6.5 Hz, 2H), 3.96 (br s, 1H), 3.62 (s, 2H), 2.79-3.00 (m, 5H), 2.00 (br s, 2H), 1.88 (br s, 1H), 1.69-1.58 (m, 6H), 0.94-1.19 (m, 5H). ¹H NMR (DMSO-d₆+D₂O, 400 MHz): δ ppm 8.69 (br s, 1H), 8.46 (s, 1H), 4.12 (d, J=6.5 Hz, 2H), 3.96 (br s, 1H), 3.62 (s, 2H), 2.79-3.00 (m, 5H), 2.00 (br s, 2H), 1.88 (br s, 1H), 1.69-1.58 (m, 7H), 0.94-1.19 (m, 5H).

### Example—S117: Synthesis of 8-cyclopentyl-2-((2-hydroxycyclopentyl)amino)-7-oxo-7,8-dihydropyrido[2,3-d]pyrimidine-6-carbonitrile (Compound No. 349)

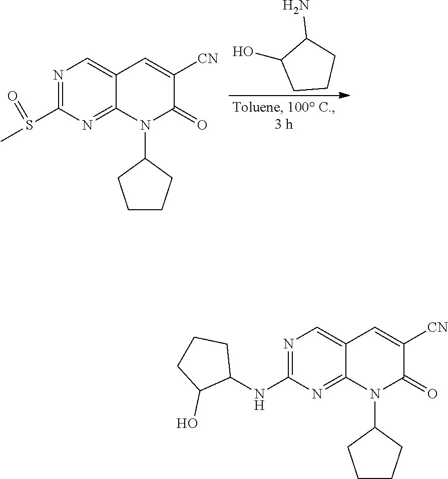

To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 2-aminocyclopentan-1-ol (37 mg, 0.36 mmol, 1.1 equiv). The resultant reaction mixture was stir at 100° C. for 3 h. Progress of the reaction was monitored by LCMS. After completion of the reaction, solvent was removed under reduced pressure to obtain crude, which was purified by trituration with methanol. LCMS: 340 [M+H]⁺, ¹H NMR (400 MHz, DMSO-d₆) δ ppm 8.66 (br s, 1H), 8.39 (s, 1H), 8.18 (br s, 1H), 5.64-5.92 (m, 1H), 4.55 (br s, 1H), 4.06 (br s, 2H), 2.32 (br s, 2H), 2.17-1.86 (m, 4H), 1.80 (br s, 2H), 1.69 (m, 4H), 1.30-1.59 (m, 2H). ¹H NMR (400 MHz, DMSO-d₆+D₂O) δ ppm 8.66 (br s, 1H), 8.39 (s, 1H), 5.64-5.92 (m, 1H), 4.06 (br s, 2H), 2.32 (br s, 2H), 2.17-1.86 (m, 4H), 1.80 (br s, 2H), 1.69 (m, 4H), 1.30-1.59 (m, 2H).

### Example—S118: Synthesis of 8-(4-hydroxytetrahydrofuran-3-yl)-2-((1-(methylsulfonyl)piperidin-4-yl)amino)-7-oxo-7,8-dihydropyrido[2,3-d]pyrimidine-6-carbonitrile (Compound No. 350)

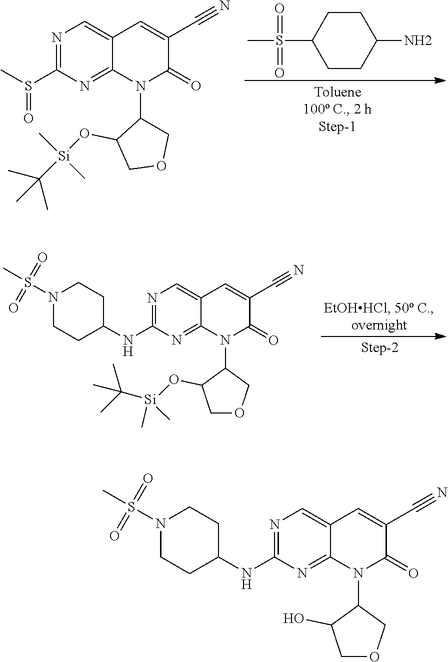

Step—1: Synthesis of 8-(4-((tert-butyldimethylsilyl)oxy)tetrahydrofuran-3-yl)-2-((1-(methylsulfonyl)piperidin-4-yl)amino)-7-oxo-7,8-dihydropyrido[2,3-d]pyrimidine-6-carbonitrile: To a stirred solution of 8-(4-((tert-butyldimethylsilyl)oxy)tetrahydrofuran-3-yl)-2-(methylsulfinyl)-7-oxo-7,8-dihydropyrido[2,3-d]pyrimidine-6-carbonitrile (100 mg, 0.23 mmol, 1 equiv) in toluene (5 mL), was added 1-(methylsulfonyl)piperidin-4-amine (45 mg, 0.25 mmol, 1.1 equiv). The resultant reaction mixture was stir at 100° C. for 2 h. Progress of the reaction was monitored by LCMS. After completion of the reaction, solvent was removed under reduced pressure to obtain crude, which was used for the next step without any further purification.

Step—2: Synthesis of 8-(4-hydroxytetrahydrofuran-3-yl)-2-((1-(methylsulfonyl)piperidin-4-yl)amino)-7-oxo-7,8-dihydropyrido[2,3-d]pyrimidine-6-carbonitrile: A solution of 8-(4-((tert-butyldimethylsilyl)oxy)tetrahydrofuran-3-yl)-2-((1-(methylsulfonyl)piperidin-4-yl)amino)-7-oxo-7,8-dihydropyrido[2,3-d]pyrimidine-6-carbonitrile (100 mg, 0.18 mmol, 1 equiv) in 1.25 M HCl in ethanol (5 mL) was allowed to stir at 50° C. for overnight. Progress of the reaction was monitored by LCMS. After completion of the reaction, solvent was removed under reduced pressure and the residue was dried under lyophiliser to obtain crude compound, which was purified by reverse phase HPLC. LCMS: 435 [M+H]⁺, ¹H NMR (DMSO-d₆, 400 MHz): δ ppm 8.71 (br s, 1H), 8.47 (s, 1H), 8.38 (br s, 1H), 5.84 (br s, 1H), 4.95 (br s, 1H), 4.24 (br s, 1H), 4.04 (m, 3H), 3.53-3.74 (m, 3H), 2.88 (m, 5H), 1.85-2.11 (m, 2H), 1.65 ppm (m, 2H). ¹H NMR (DMSO-d₆+D₂O, 400 MHz): δ ppm 8.71 (br s, 1H), 8.47 (s, 1H), 5.84 (br s, 1H), 4.95 (br s, 1H), 4.24 (br s, 1H), 4.04 (m, 3H), 3.53-3.74 (m, 3H), 2.88 (m, 5H), 1.85-2.11 (m, 2H), 1.65 ppm (m, 2H).

### Example—S119: Synthesis of 8-(sec-butyl)-2-((1-(methylsulfonyl)piperidin-4-yl)amino)-7-oxo-7,8-dihydropyrido[2,3-d]pyrimidine-6-carbonitrile (Compound No. 351)

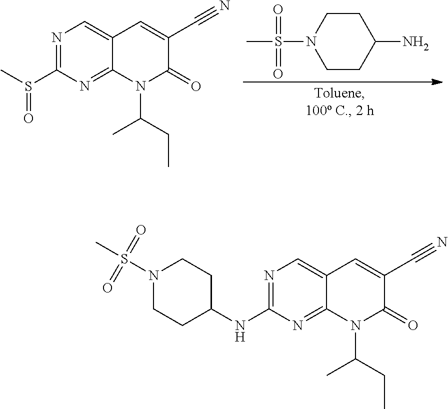

To a stirred solution of 8-(sec-butyl)-2-(methylsulfinyl)-7-oxo-7,8-dihydropyrido[2,3-d]pyrimidine-6-carbonitrile (80 mg, 0.27 mmol, 1 equiv) in toluene (5 mL), was added 1-(methylsulfonyl)piperidin-4-amine (54 mg, 0.3 mmol, 1.1 equiv). The resultant reaction mixture was stir at 100° C. for 2 h. Progress of the reaction was monitored by LCMS. After completion of the reaction, solvent was removed under reduced pressure to obtain crude, which was purified by trituration with methanol. LCMS: 405 [M+H]+¹H NMR (DMSO-d₆, 400 MHz): δ ppm 8.67 (s, 1H), 8.41 (s, 1H), 8.29 (br s, 1H), 5.43 (d, J=6.1 Hz, 1H), 3.95 (br s, 1H), 3.58 (s, 2H), 2.76-3.02 (m, 5H), 2.17 (br s, 1H), 2.07-1.95 (m, 3H), 1.66 (br s, 2H), 1.50 (d, J=6.6 Hz, 3H), 0.78 ppm (t, J=7.5 Hz, 3H). ¹H NMR (DMSO-d₆+D₂O, 400 MHz): δ ppm 8.67 (s, 1H), 8.41 (s, 1H), 5.43 (d, J=6.1 Hz, 1H), 3.95 (br s, 1H), 3.58 (s, 2H), 2.76-3.02 (m, 5H), 2.17 (br s, 1H), 2.07-1.95 (m, 3H), 1.66 (br s, 2H), 1.50 (d, J=6.6 Hz, 3H), 0.78 ppm (t, J=7.5 Hz, 3H).

### Example—S120: Synthesis of 2-((1-(methylsulfonyl)piperidin-4-yl)amino)-7-oxo-8-(pentan-3-yl)-7,8-dihydropyrido[2,3-d]pyrimidine-6-carbonitrile (Compound No. 352)

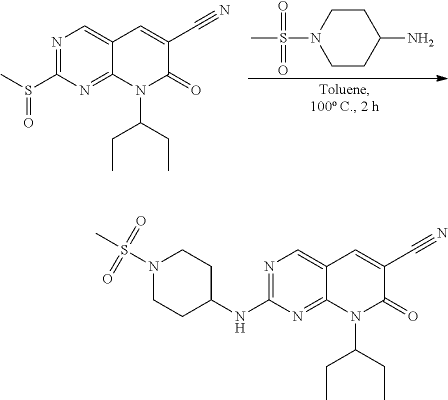

To a stirred solution of 2-(methylsulfinyl)-7-oxo-8-(pentan-3-yl)-7,8-dihydropyrido[2,3-d]pyrimidine-6-carbonitrile (80 mg, 0.26 mmol, 1 equiv) in toluene (5 mL), was added 1-(methylsulfonyl)piperidin-4-amine (52 mg, 0.28 mmol, 1.1 equiv). The resultant reaction mixture was stir at 100° C. for 2 h. Progress of the reaction was monitored by LCMS. After completion of the reaction, solvent was removed under reduced pressure to obtain crude, which was purified by trituration with methanol. LCMS: 419 [M+H]+¹H NMR (400 MHz, DMSO-d₆): δ ppm 8.70 (s, 1H), 8.45 (s, 1H), 5.31 (br s, 1H), 3.94 (br s, 1H), 3.60 (s, 2H), 2.73-2.98 (m, 5H), 2.22 (br s, 2H), 2.09 (m, 2H), 1.90 (br s, 2H), 1.65 (3, 2H), 0.77 ppm (t, J=7.2 Hz, 6H).

### Example—S121: Synthesis of 8-((1S,2S,4R)-bicyclo[2.2.1]heptan-2-yl)-2-(1-(methylsulfonyl)piperidin-4-ylamino)-7-oxo-7,8-dihydropyrido[2,3-d]pyrimidine-6-carbonitrile (Compound No. 353), 8-((1R,2R,4S)-bicyclo[2.2.1]heptan-2-yl)-2-(1-(methylsulfonyl)piperidin-4-ylamino)-7-oxo-7,8-dihydropyrido[2,3-d]pyrimidine-6-carbonitrile (Compound No. 386) and 8-((1S,2S,4R)-bicyclo[2.2.1]heptan-2-yl)-2-(1-(methylsulfonyl)piperidin-4-ylamino)-7-oxo-7,8-dihydropyrido[2,3-d]pyrimidine-6-carbonitrile (Compound No. 387)

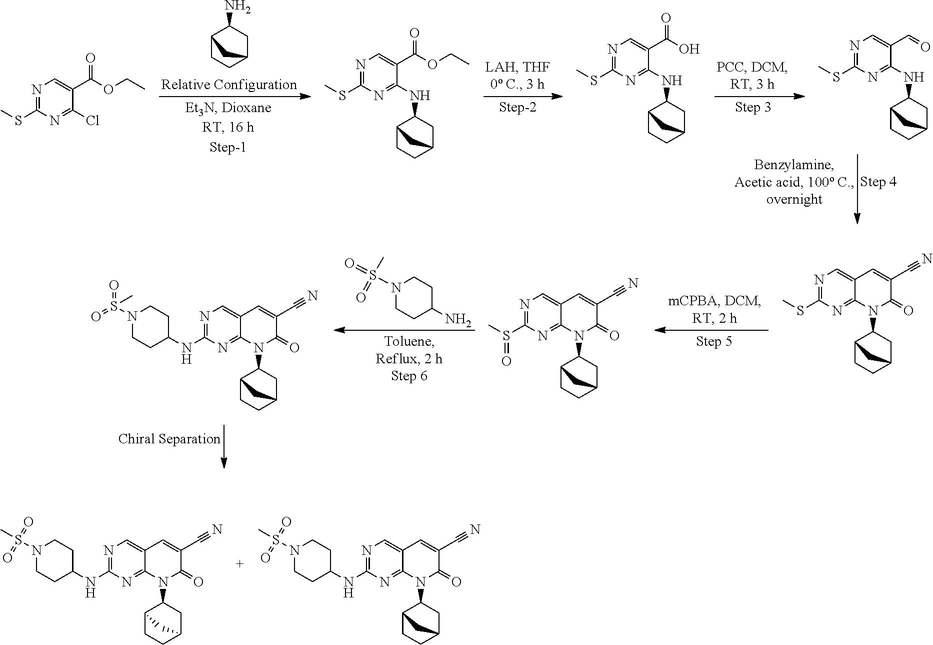

Step—1: Synthesis of ethyl 4-((1S,2S,4R)-bicyclo[2.2.1]heptan-2-ylamino)-2-(methylthio)pyrimidine-5-carboxylate: To a stirred solution of ethyl 4-chloro-2-(methylthio)pyrimidine-5-carboxylate (3000 mg, 12.89 mmol, 1 equiv) in 1,4-Dioxane (30 mL), was added Et₃N (5.4 mL, 38.68 mmol, 3.0 equiv) and (1S,2S,4R)-bicyclo[2.2.1]heptan-2-amine (relative configuration) (2100 mg, 14.18 mmol, 1.1 equiv) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain the desired product.

Step—2: Synthesis of (4-((1S,2S,4R)-bicyclo[2.2.1]heptan-2-ylamino)-2-(methylthio)pyrimidin-5-yl)methanol: To a stirred solution of ethyl 4-((1S,2S,4R)-bicyclo[2.2.1]heptan-2-ylamino)-2-(methylthio)pyrimidine-5-carboxylate (4000 mg, 13.01 mmol, 1 equiv) in THF (50 mL), was added LAH (990 mg, 26.02 mmol, 2 equiv) at 0° C. The reaction mixture was allowed to stir at 0° C. for 3 h. Progress of the reaction was monitored by TLC and LCMS. After completion of the reaction, the reaction mixture was quenched with saturated solution of sodium hydroxide (100 mL) at 0° C. Obtained residue was filtered through cealite bed and washed with ethyl acetate (50 mL). Filtrate was extracted with ethyl acetate (150 mL×2). Organic layer was washed with water (100 mL) and brine solution (100 mL). Organic layer was dried over anhydrous sodium sulphate and concentrated under reduced pressure to obtain the product.

Step—3: Synthesis of 4-((1S,2S,4R)-bicyclo[2.2.1]heptan-2-ylamino)-2-(methylthio)pyrimidine-5-carbaldehyde: To a stirred solution of (4-((1S,2S,4R)-bicyclo[2.2.1]heptan-2-ylamino)-2-(methylthio)pyrimidin-5-yl)methanol (3000 mg, 11.30 mmol, 1 equiv) in DCM (30 mL), was added PCC (2920 mg, 13.56 mmol, 1.2 equiv) at RT. The reaction mixture was then allowed to stir at RT for 3 h. Progress of the reaction was monitored by TLC and LCMS. After completion of the reaction, the reaction mixture was passed through celite bed, filtrate obtain was diluted with DCM (150 mL) and washed with water (100 mL), brine solution (100 mL). Organic layer was dried over anhydrous sodium sulphate and concentrated under reduced pressure to obtain crude, which was purified by normal phase combi flash to obtain the desired product.

Step—4: Synthesis of 8-((1S,2S,4R)-bicyclo[2.2.1]heptan-2-yl)-2-(methylthio)-7-oxo-7,8-dihydropyrido[2,3-d]pyrimidine-6-carbonitrile: To a stirred solution of 4-((1S,2S,4R)-bicyclo[2.2.1]heptan-2-ylamino)-2-(methylthio)pyrimidine-5-carbaldehyde (2300 mg, 8.75 mmol, 1 equiv) in Acetic acid (15 mL), was added Cyanoacetic acid (970 mg, 5.33 mmol, 1.3 equiv) and Benzyl amine (0.09 mL, 0.87, 0.1 equiv). The reaction mixture was allowed to stir at 100° C. for overnight. Progress of the reaction was monitored by TLC and LCMS. After completion of the reaction, the reaction mixture was diluted with water (50 mL) and extracted with ethyl acetate (100 mL×2). Organic layer was washed with water (100 mL) and brine solution (100 mL). Organic layer was dried over anhydrous sodium sulphate and concentrated under reduced pressure to obtain crude compound, which was purified by normal phase combi-flash to obtain the product.

Step—5: Synthesis of 8-((1S,2S,4R)-bicyclo[2.2.1]heptan-2-yl)-2-(methylsulfinyl)-7-oxo-7,8-dihydropyrido[2,3-d]pyrimidine-6-carbonitrile: To a stirred solution of 8-((1S,2S,4R)-bicyclo[2.2.1]heptan-2-yl)-2-(methylthio)-7-oxo-7,8-dihydropyrido[2,3-d]pyrimidine-6-carbonitrile (600 mg, 1.93 mmol, 1 equiv) in DCM (6 mL), was added m-CPBA (430 mg, 2.5 mmol, 1.3 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain the product.

Step—6: Synthesis of 8-((1r,4r)-bicyclo[2.2.1]heptan-1-yl)-2-((1-(methylsulfonyl)piperidin-4-yl)amino)-7-oxo-7,8-dihydropyrido[2,3-d]pyrimidine-6-carbonitrile: To the stirred solution of 1-(methylsulfonyl)piperidin-4-amine (78 mg, 0.43 mmol, 0.5 equiv) in Toluene (5 mL) and DCM (5 mL) was added 8-((1S,2S,4R)-bicyclo[2.2.1]heptan-2-yl)-2-(methylsulfinyl)-7-oxo-7,8-dihydropyrido[2,3-d]pyrimidine-6-carbonitrile (0.3 g, 0.87 mmol, 1.0 equiv) and was stirred for overnight (until the completion of the reaction). After completion of the reaction, solvents were evaporated under vacuum and the crude mixture was purified using reverse phase HPLC. Racemate was purified by chiral chromatography to give the desired products. LCMS: 443 [M+H]⁺; ¹H NMR (400 MHz, DMSO-d₆+D₂O, VT @ 90° C.) δ ppm 8.64 (br s, 1H) 8.34 (s, 1H) 5.14 (br s, 1H) 3.93 (br s, 1H) 3.62 (d, J=9.2 Hz, 2H) 2.86 (s, 5H) 2.67 (br s, 1H) 2.45 (br s, 2H) 2.27-2.41 (m, 2H) 2.02 (br s, 3H) 1.69 (br s, 2H) 1.57 (br s, 2H) 1.15-1.28 (m, 2H).

### Example—S122: Synthesis of 8-((S,2S,4R)-bicyclo[2.2.1]heptan-2-yl)-7-oxo-2-(1,2,3,4-tetrahydroisoquinolin-6-ylamino)-7,8-dihydropyrido[2,3-d]pyrimidine-6-carbonitrile (Compound No. 354)

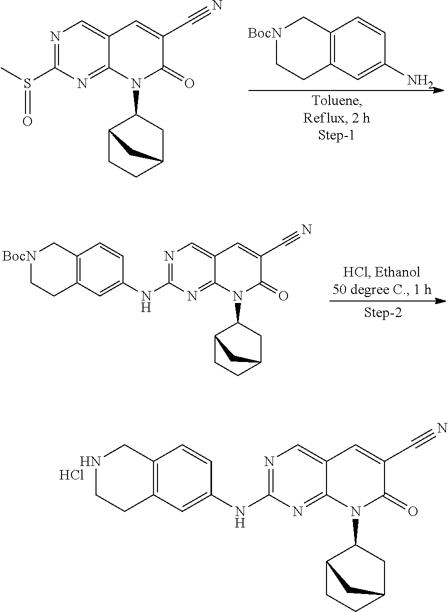 [SMILES: CS(=O)c1ncc2cc(C#N)c(=O)n([C@H]3C[C@@H]4CC[C@H]3C4)c2n1 ; CC(C)(C)OC(=O)N1CCc2cc(N)ccc2C1 ; CC(C)(C)OC(=O)N1CCc2cc(Nc3ncc4cc(C#N)c(=O)n([C@H]5C[C@@H]6CC[C@H]5C6)c4n3)ccc2C1 ; Cl ; N#Cc1cc2cnc(Nc3ccc4c(c3)CCNC4)nc2n([C@H]2C[C@@H]3CC[C@H]2C3)c1=O]

Step—1: Synthesis of tert-butyl 6-(8-((1S,2S,4R)-bicyclo[2.2.1]heptan-2-yl)-6-cyano-7-oxo-7,8-dihydropyrido[2,3-d]pyrimidin-2-ylamino)-3,4-dihydroisoquinoline-2(1H)-carboxylate: To a stirred solution of 8-((1S,2S,4R)-bicyclo[2.2.1]heptan-2-yl)-2-(methylsulfinyl)-7-oxo-7,8-dihydropyrido[2,3-d]pyrimidine-6-carbonitrile (200 mg, 0.60 mmol, 1 equiv) in toluene (5 mL), was added tert-butyl 6-amino-3,4-dihydroisoquinoline-2(1H)-carboxylate (150 mg, 0.60 mmol, 1.0 equiv). The resultant reaction mixture was allowed to stir at 100° C. for 1 h. Progress of the reaction was monitored by LCMS. After completion of the reaction, solid observed was filtered and dried under vacuum to obtain crude compound, which was purified by recrystallization with methanol to obtain the product.

Step—2: Synthesis of 8-((1S,2S,4R)-bicyclo[2.2.1]heptan-2-yl)-7-oxo-2-(1,2,3,4-tetrahydroisoquinolin-6-ylamino)-7,8-dihydropyrido[2,3-d]pyrimidine-6-carbonitrile (Relative configuration): A solution of tert-butyl 6-((8-((1r,4r)-bicyclo[2.2.1]heptan-1-yl)-6-cyano-7-oxo-7,8-dihydropyrido[2,3-d]pyrimidin-2-yl)amino)-3,4-dihydroisoquinoline-2(1H)-carboxylate (200 mg, 0.39 mmol, 1 equiv) in 1.25 M HCl in ethanol (4 mL) was allowed to stir for 1 h at 50° C. Progress of the reaction was monitored by LCMS. After completion of the reaction, the solvent was evaporated on rotary evaporator and was purified using prep HPLC to give the desired product. LCMS: 413 [M+H]⁺; ¹H NMR (400 MHz, DMSO-d₆) δ ppm 8.80 (s, 1H) 8.49 (s, 1H) 8.32 (br s, 1H) 7.60 (br s, 1H) 7.46 (br s, 1H) 7.13 (d, J=8.7 Hz, 1H) 5.21 (br s, 1H) 4.07 (br s, 2H) 3.19 (br s, 2H) 2.88 (br s, 2H) 2.67 (br s, 1H) 2.34 (d, J=10.5 Hz, 2H) 2.07 (br s, 2H) 1.63-1.78 (m, 2H) 1.56 (d, J=11.8 Hz, 2H) 1.14-1.27 (m, 2H).

### Example—S123: Synthesis of 8-cyclopentyl-2-((1-(isopropylsulfonyl)piperidin-4-yl)amino)-7-oxo-7,8-dihydropyrido[2,3-d]pyrimidine-6-carbonitrile (Compound No. 355)

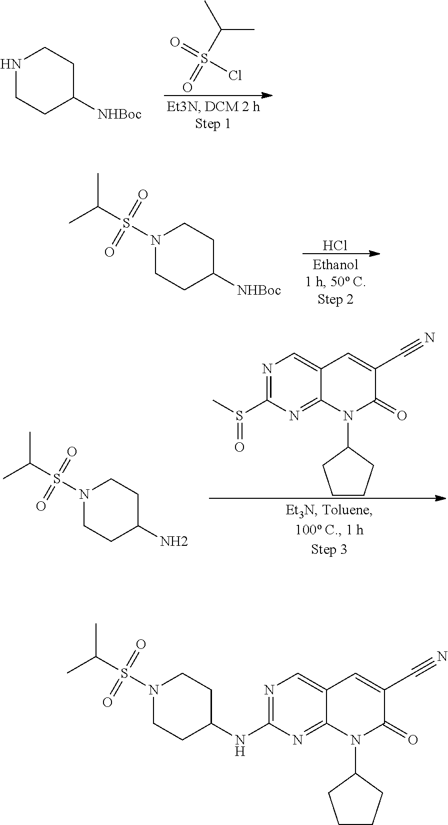

Step—1: Synthesis of tert-butyl (1-(isopropylsulfonyl)piperidin-4-yl)carbamate: To the stirred solution of tert-butyl piperidin-4-ylcarbamate (1.0, 4.99 mmol, 1.0 equiv) in DCM (15 mL) at 0 C was added Et₃N (1.24 mL, 6.53 mmol, 1.3 equiv) followed by the addition of propane-2-sulfonyl chloride (0.67 mL, 5.99 mmol, 1.2 equiv). The reaction mixture was then allowed to warm to RT and stirred for another hour until the completion of the reaction. After completion of the reaction, water (10 mL) was added to the reaction mixture and extracted with DCM (20 mL×3). Combined organic layer was washed with water, brine and dried over sodium sulfate. Volatiles were removed under vacuum to give the desired product.

Step—2: Synthesis of 1-(isopropylsulfonyl)piperidin-4-amine: Tert-butyl (1-(isopropylsulfonyl)piperidin-4-yl)carbamate (1.0 g, 3.3 mmol, 1.0 equiv) was taken in the reaction bottle and HCL (10 mL, 1.25 M in Ethanol) was added and reaction mixture was stirred at 50 C for 1 h until the completion of the reaction. Solvent were removed under vacuum, residual solvent were evaporated on lyophilizer to give the desired product.

Step—3: Synthesis of 8-cyclopentyl-2-((1-(isopropylsulfonyl)piperidin-4-yl)amino)-7-oxo-7,8-dihydropyrido[2,3-d]pyrimidine-6-carbonitrile: To the stirred solution of 1-(isopropylsulfonyl)piperidin-4-amine (0.3 g, 1.24 mmol, 1.0 equiv) in Toluene (5 mL) was added triethylamine (0.35 mL, 2.47 mmol, 2.0 equiv) at RT followed by the addition of 8-cyclopentyl-2-(methylsulfinyl)-7-oxo-7,8-dihydropyrido[2,3-d]pyrimidine-6-carbonitrile (0.37 g, 1.24 mmol). The reaction mixture was then stirred at 100 C for 1 h until the completion of the reaction mixture. After completion of the reaction, toluene was evaporated under vacuum and product was purified using reverse phase HPLC to give the desired product. LCMS: 445 [M+H]+; ¹H NMR (400 MHz, DMSO-d₆) δ ppm 8.66 (br s, 1H) 8.36 (s, 1H) 5.76 (br s, 1H) 3.98 (br s, 1H) 3.68 (d, J=12.7 Hz, 2H) 3.25-3.30 (m, 1H) 3.02 (t, J=11.1 Hz, 2H) 2.24 (br s, 2H) 1.86-2.00 (m, 4H) 1.78 (br s, 2H) 1.50-1.71 (m, 4H) 1.23 (d, J=6.5 Hz, 6H)

### Example—S124: Synthesis of 8-cyclopentyl-2-((1-(cyclopropylsulfonyl)piperidin-4-yl)amino)-7-oxo-7,8-dihydropyrido[2,3-d]pyrimidine-6-carbonitrile (Compound No. 356)

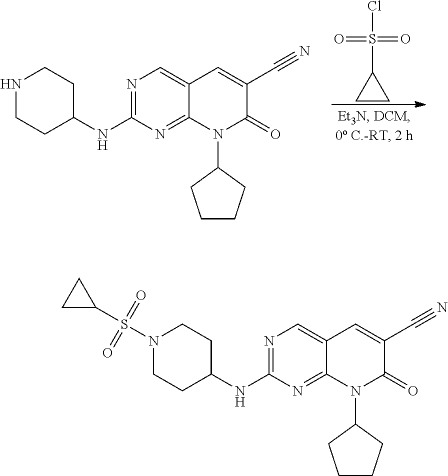

To a stirred solution of 8-cyclopentyl-7-oxo-2-(piperidin-4-ylamino)-7,8-dihydropyrido[2,3-d]pyrimidine-6-carbonitrile (80 mg, 0.21 mmol, 1 equiv) in DCM (5 mL), was added ET₃N (0.1 mL, 0.63 mmol, 3 equiv) and cyclopropanesulfonyl chloride (45 mg, 0.32 mmol, 1.5 equiv) at 0° C. Raised the temp. to RT and the reaction mixture was allowed to stir for 2 h. Progress of the reaction was monitored by LCMS. After completion of the reaction, the reaction mixture was diluted with water (15 mL) and extracted with DCM (50 mL). Organic layer was washed with water (50 mL), brine solution (50 mL). Organic layer was dried over anhydrous sodium sulphate and concentrated under reduced pressure to obtain crude, which was purified by reverse phase HPLC. LCMS: 443 [M+H]⁺, ¹H NMR (DMSO-d₆, 400 MHz): δ ppm 8.69 (s, 1H), 8.42 (s, 1H), 8.33 (br s, 1H), 5.79 (br s, 1H), 4.00 (br s, 1H), 3.66 (s, 2H), 3.13-2.98 (m, 2H), 2.57 (br s, 1H), 2.33 (s, 2H), 2.05-1.98 (br s, 4H), 1.80 (br s, 2H), 1.69-1.67 (m, 4H), 0.80-1.08 (m, 4H). ¹H NMR (DMSO-d₆+D20,400 MHz): δ ppm 8.69 (s, 1H), 8.42 (s, 1H), 5.79 (br s, 1H), 4.00 (br s, 1H), 3.66 (s, 2H), 3.13-2.98 (m, 2H), 2.57 (br s, 1H), 2.33 (s, 2H), 2.05-1.98 (br s, 4H), 1.80 (br s, 2H), 1.69-1.67 (m, 4H), 0.80-1.08 (m, 4H).

### Example—S125: Synthesis of 8-cyclopentyl-2-((1-(ethylsulfonyl)piperidin-4-yl)amino)-7-oxo-7,8-dihydropyrido[2,3-d]pyrimidine-6-carbonitrile (Compound No. 357)

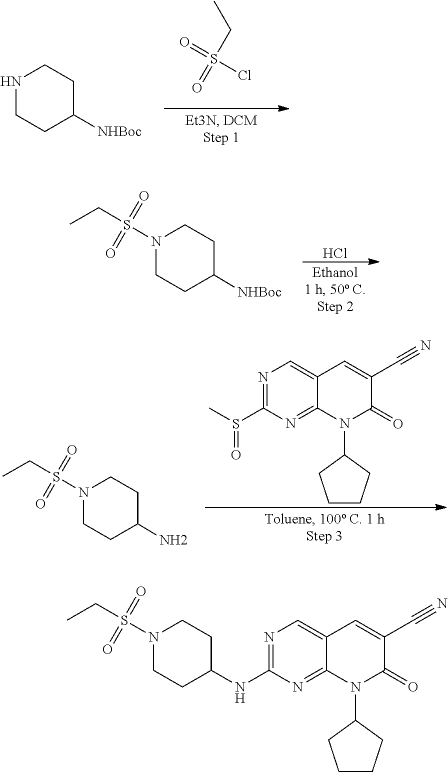

Step—1: Synthesis of tert-butyl (1-(isopropylsulfonyl)piperidin-4-yl)carbamate: To the stirred solution of tert-butyl piperidin-4-ylcarbamate (1.0 g, 5.0 mmol, 1.0 equiv) in DCM (10 ml) at 0 C was added Et3N (1.24 mL, 6.5 mmol, 1.3 mmol) followed by the addition of ethanesulfonyl chloride (0.77 g, 6.0 mmol, 1.2 equiv). The reaction mixture was then allowed to warm to RT and stirred for another hour until the completion of the reaction. After completion of the reaction, water (10 mL) was added to the reaction mixture and extracted with DCM (20 mL×3). Combined organic layer was washed with water, brine and dried over sodium sulfate. Volatiles were removed under vacuum to give the desired product.

Step—2: Synthesis of 1-(ethylsulfonyl)piperidin-4-amine: Tert-butyl (1-(isopropylsulfonyl)piperidin-4-yl)carbamate was taken in the reaction bottle and HCL (0.4 g, 2.08 mmol, 1.0 equiv) was added and reaction mixture was stirred at 50° C. for 1 h until the completion of the reaction. Solvent were removed under vacuum, residual solvent were evaporated on lyophilizer to give the desired product.

Step—3: Synthesis of 8-cyclopentyl-2-((1-(ethylsulfonyl)piperidin-4-yl)amino)-7-oxo-7,8-dihydropyrido[2,3-d]pyrimidine-6-carbonitrile: To the stirred solution of 1-(ethylsulfonyl)piperidin-4-amine (0.23 g, 1.39 mmol, 1.0 equiv) in Toluene (5 mL) was added triethylamine (0.21 mL, 1.52 mmol, 1.1 equiv) at RT followed by the addition of 8-cyclopentyl-2-(methylsulfinyl)-7-oxo-7,8-dihydropyrido[2,3-d]pyrimidine-6-carbonitrile (0.3 g, 1.39 mmol, 1.0 equiv). The reaction mixture was then stirred at 100 C for 1 h until the completion of the reaction mixture. After completion of the reaction, toluene was evaporated under vacuum and product was purified using reverse phase HPLC to give the desired product. LCMS: 431 [M+H]⁺; ¹H NMR (400 MHz, DMSO-d₆) δ ppm 8.66 (br s, 1H) 8.36 (s, 1H) 5.76 (br s, 1H) 3.94 (d, J=12.2 Hz, 1H) 3.63 (d, J=12.2 Hz, 2H) 2.84-3.12 (m, 4H) 2.23 (br s, 2H) 1.95 (br s, 3H) 1.78 (br s, 2H) 1.63 (br s, 3H) 1.36 (s, 2H) 1.16-1.29 (m, 4H)

### Example—S126: Synthesis of 8-cyclopentyl-7-oxo-2-((1-((2,2,2-trifluoroethyl)sulfonyl)piperidin-4-yl)amino)-7,8-dihydropyrido[2,3-d]pyrimidine-6-carbonitrile (Compound No. 358)

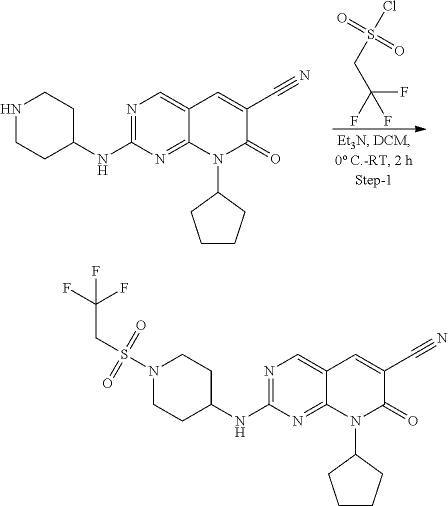 [SMILES: N#Cc1cc2cnc(NC3CCNCC3)nc2n(C2CCCC2)c1=O ; O=S(=O)(Cl)CC(F)(F)F ; N#Cc1cc2cnc(NC3CCN(S(=O)(=O)CC(F)(F)F)CC3)nc2n(C2CCCC2)c1=O]

To a stirred solution of 8-cyclopentyl-7-oxo-2-(piperidin-4-ylamino)-7,8-dihydropyrido[2,3-d]pyrimidine-6-carbonitrile (80 mg, 0.21 mmol, 1 equiv) in DCM (5 mL), was added ET₃N (0.1 mL, 0.63 mmol, 3 equiv) and 2,2,2-trifluoroethane-1-sulfonyl chloride (59 mg, 0.32 mmol, 1.5 equiv) at 0° C. Reaction mixture was allowed to stir for 2 h at RT. Progress of the reaction was monitored by LCMS. After completion of the reaction, the reaction mixture was diluted with water (15 mL) and extracted with DCM (50 mL). Organic layer was washed with water (50 mL), brine solution (50 mL). Organic layer was dried over anhydrous sodium sulphate and concentrated under reduced pressure to obtain crude, which was purified by reverse phase HPLC. LCMS: 485 [M+H]⁺, ¹H NMR (DMSO-d₆, 400 MHz): δ ppm 8.70 (s, 1H), 8.42 (s, 1H), 8.33 (br s, 1H), 5.79 (br s, 1H), 4.29-4.50 (m, 2H), 4.00 (br s, 1H), 3.70 (s, 2H), 3.03 (br s, 2H), 2.32-2.28 (m, 2H), 1.99 (br s, 4H), 1.80 (br s, 2H), 1.68-1.65 ppm (br s, 4H).

### Example—S127: Synthesis of 8-cyclopentyl-2-((1-((difluoromethyl)sulfonyl)piperidin-4-yl)amino)-7-oxo-7,8-dihydropyrido[2,3-d]pyrimidine-6-carbonitrile (Compound No. 359)

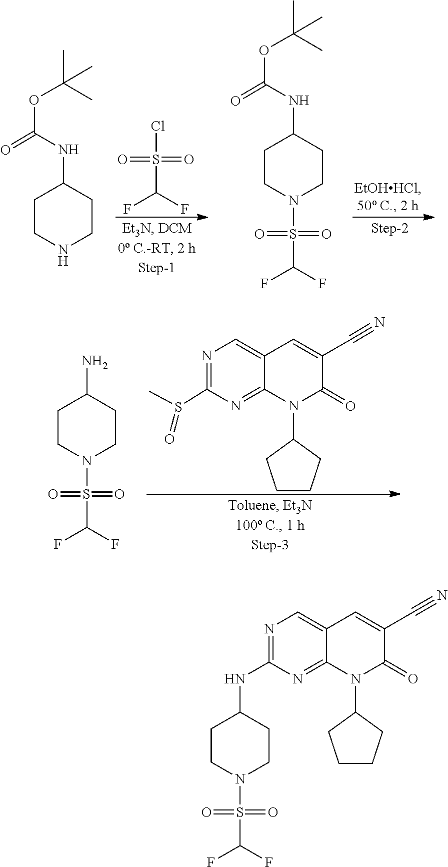

Step—1: Synthesis of tert-butyl (1-((difluoromethyl)sulfonyl)piperidin-4-yl)carbamate: To a stirred solution of tert-butyl piperidin-4-ylcarbamate (500 mg, 2.5 mmol, 1 equiv) in DCM (5 mL), was added ET₃N (0.5 mL, 3.75 mmol, 1.5 equiv) and difluoromethanesulfonyl chloride (453 mg, 3 mmol, 1.2 equiv) at 0° C. The reaction mixture was allowed to stir at RT for 2 h. Progress of the reaction was monitored by TLC and LCMS. After completion of the reaction, the reaction mixture was diluted with water (20 mL) and extracted with DCM (50 mL). Organic layer was washed with water (20 mL), brine solution (20 mL). Organic layer was dried over anhydrous sodium sulphate and concentrated under reduced pressure to obtain crude, which was used for the next step without any further purification.

Step—2: Synthesis of 1-((difluoromethyl)sulfonyl)piperidin-4-amine: A solution of tert-butyl (1-((difluoromethyl)sulfonyl)piperidin-4-yl)carbamate (250 mg, 0.79 mmol, 1 equiv) in 1.25 M HCl in ethanol (5 mL) was allowed to stir at 50° C. for 2 h. Progress of the reaction was monitored by LCMS. After completion of the reaction, solvent was removed under reduced pressure to obtain crude, which was used for the next step without any further purification.

Step—3: Synthesis of 8-cyclopentyl-2-((1-((difluoromethyl)sulfonyl)piperidin-4-yl)amino)-7-oxo-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (150 mg, 0.49 mmol, 1 equiv) in toluene (5 mL), was added 1-((difluoromethyl)sulfonyl)piperidin-4-amine (137 mg, 0.54 mmol, 1.1 equiv) and ET₃N (0.2 mL, 1.47 mmol, 3 equiv). The resultant reaction mixture was stir at 100° C. for 1 h. Progress of the reaction was monitored by LCMS. After completion of the reaction, solvent was removed under reduced pressure to obtain crude, which was purified by reverse phase HPLC. LCMS: 453 [M+H]⁺, ¹H NMR (DMSO-d₆, 400 MHz): 8.70 (br s, 1H), 8.42 (s, 1H), 8.35 (br s, 1H), 7.02 (s, 1H), 5.81 (br s, 1H), 4.07 (br s, 1H), 3.82-3.79 (d, J=9.2 Hz, 2H), 3.26 (t, J=11.8 Hz, 2H), 2.33-2.26 (br s, 2H), 2.05-1.99 (br s, 4H), 1.80 (br s, 2H), 1.66 (d, J=9.2 Hz, 4H).

### Example—S128: Synthesis of 8-cyclopentyl-7-oxo-2-((1-tosylpiperidin-4-yl)amino)-7,8-dihydropyrido[2,3-d]pyrimidine-6-carbonitrile (Compound No. 360)

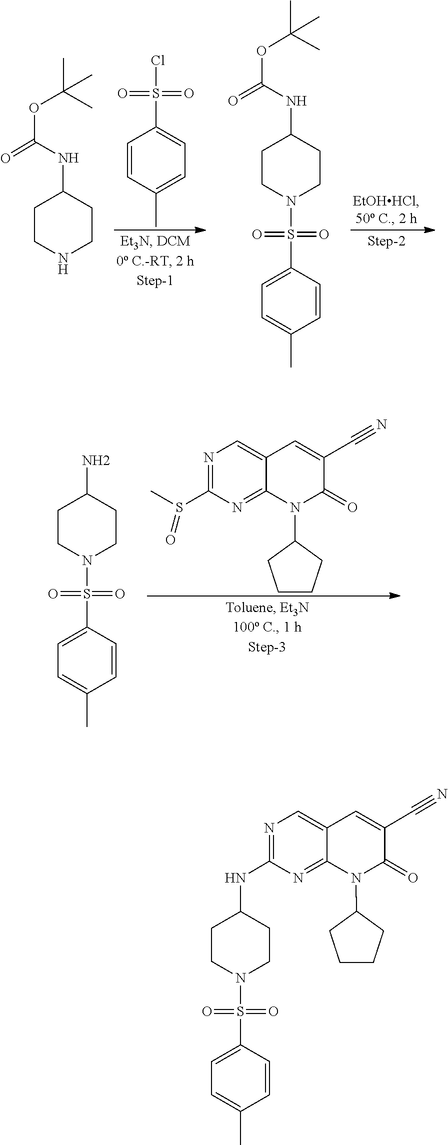

Step—1: Synthesis of tert-butyl (1-tosylpiperidin-4-yl)carbamate: To a stirred solution of tert-butyl piperidin-4-ylcarbamate (500 mg, 2.5 mmol, 1 equiv) in DCM (5 mL), was added ET₃N (0.5 mL, 3.75 mmol, 1.5 equiv) and 4-methylbenzenesulfonyl chloride (585 mg, 3 mmol, 1.2 equiv) at 0° C. The reaction mixture was allowed to stir at RT for 2 h. Progress of the reaction was monitored by TLC and LCMS. After completion of the reaction, the reaction mixture was diluted with water (30 mL) and extracted with DCM (100 mL). Organic layer was washed with water (50 mL), brine solution (50 mL). Organic layer was dried over anhydrous sodium sulphate and concentrated under reduced pressure to obtain crude, which was used for the next step without any further purification.

Step—2: Synthesis of 1-tosylpiperidin-4-amine: A solution of tert-butyl (1-tosylpiperidin-4-yl) carbamate (200 mg, 0.56 mmol, 1 equiv) in 1.25 M HCl in ethanol (5 mL) was allowed to stir at 50° C. for 2 h. Progress of the reaction was monitored by LCMS. After completion of the reaction, solvent was removed under reduced pressure to obtain crude, which was used for the next step without any further purification.

Step—3: Synthesis of 8-cyclopentyl-7-oxo-2-((1-tosylpiperidin-4-yl)amino)-7,8-dihydropyrido[2,3-d]pyrimidine-6-carbonitrile: To a stirred solution of 8-cyclopentyl-2-(methylsulfinyl)-7-oxo-7,8-dihydropyrido[2,3-d]pyrimidine-6-carbonitrile (150 mg, 0.49 mmol, 1 equiv) in toluene (5 mL), was added 1-tosylpiperidin-4-amine (158 mg, 0.54 mmol, 1.1 equiv) and ET₃N (0.2 mL, 1.47 mmol, 3 equiv). The resultant reaction mixture was stir at 100° C. for 1 h. Progress of the reaction was monitored by LCMS. After completion of the reaction, solvent was removed under reduced pressure to obtain crude, which was purified by reverse phase HPLC. LCMS: 493 [M+H]⁺, ¹H NMR (400 MHz, DMSO-d₆): δ 8.65 (s, 1H), 8.39 (s, 1H), 8.28 (br s, 1H), 7.65 (d, J=8.3 Hz, 2H), 7.45 (d, J=7.9 Hz, 2H), 5.71 (br s, 1H), 3.86 (br s, 1H), 3.62-3.59 (br s, 2H), 2.52-2.63 (m, 2H), 2.43 (s, 3H), 2.33 (br s, 2H), 1.96-1.93 (br s, 2H), 1.76 (br s, 2H), 1.66 (d, J=10.1 Hz, 2H), 1.57 (br s, 4H).

### Example—S129: Synthesis of 8-cyclopentyl-2-((1-((4-fluorophenyl)sulfonyl)piperidin-4-yl)amino)-7-oxo-7,8-dihydropyrido[2,3-d]pyrimidine-6-carbonitrile (Compound No. 361)

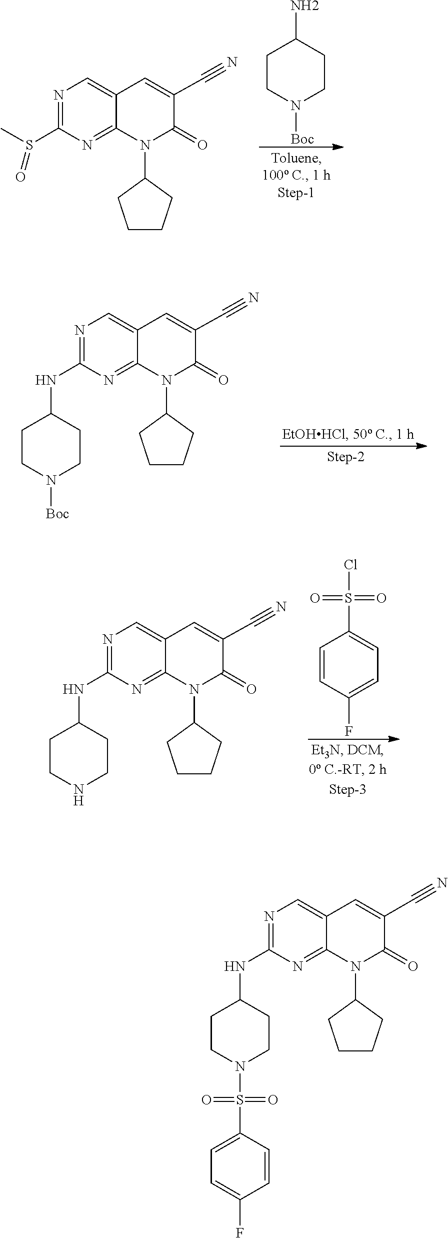

Step—1: Synthesis of tert-butyl 4-((6-cyano-8-cyclopentyl-7-oxo-7,8-dihydropyrido[2,3-d]pyrimidin-2-yl)amino)piperidine-1-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added tert-butyl 4-aminopiperidine-1-carboxylate (72 mg, 0.36 mmol, 1.1 equiv). The resultant reaction mixture was stir at 100° C. for 1 h. Progress of the reaction was monitored by LCMS. After completion of the reaction, solvent was removed under reduced pressure to obtain crude, which was used for the next step without any further purification.

Step—2: Synthesis of 8-cyclopentyl-7-oxo-2-(piperidin-4-ylamino)-7,8-dihydropyrido[2,3-d]pyrimidine-6-carbonitrile: A solution of tert-butyl 4-((6-cyano-8-cyclopentyl-7-oxo-7,8-dihydropyrido[2,3-d]pyrimidin-2-yl)amino)piperidine-1-carboxylate (100 mg, 0.22 mmol, 1 equiv) in 1.25 M HCl in ethanol (5 mL) was allowed to stir at 50° C. for 1 h. Progress of the reaction was monitored by LCMS. After completion of the reaction, solvent was removed under reduced pressure to obtain crude, which was used for the next step without any further purification.

Step—3: Synthesis of 8-cyclopentyl-2-((1-((4-fluorophenyl)sulfonyl)piperidin-4-yl)amino)-7-oxo-7,8-dihydropyrido[2,3-d]pyrimidine-6-carbonitrile: To a stirred solution of 8-cyclopentyl-7-oxo-2-(piperidin-4-ylamino)-7,8-dihydropyrido[2,3-d]pyrimidine-6-carbonitrile (80 mg, 0.21 mmol, 1 equiv) in DCM (5 mL), was added ET₃N (0.04 mL, 0.32 mmol, 1.5 equiv) and 4-fluorobenzenesulfonyl chloride (50 mg, 0.32 mmol, 1.2 equiv) at 0° C. The reaction mixture was allowed to stir at RT for 2 h. Progress of the reaction was monitored by LCMS. After completion of the reaction, the reaction mixture was diluted with water (15 mL) and extracted with DCM (50 mL). Organic layer was washed with water (50 mL), brine solution (50 mL). Organic layer was dried over anhydrous sodium sulphate and concentrated under reduced pressure to obtain crude, which was purified by reverse phase HPLC. LCMS: 497 [M+H]⁺, ¹H NMR (400 MHz, DMSO-d₆): ppm 8.65 (s, 1H), 8.40 (s, 1H), 8.29 (br s, 1H), 7.85 (dd, J=8.6, 5.0 Hz, 2H), 7.47 (t, J=8.8 Hz, 2H), 5.73 (br s, 1H), 3.87 (br s, 1H), 3.64-3.61 (br s, 2H), 2.54-2.72 (m, 2H), 2.23 (br s, 2H), 1.96 (br s, 2H), 1.77 (br s, 2H), 1.67-1.58, (br s, 6H).

### Example—S130: Synthesis of 8-cyclopentyl-2-((3R,4R)-3-fluoro-1-(methylsulfonyl)piperidin-4-ylamino)-7-oxo-7,8-dihydropyrido[2,3-d]pyrimidine-6-carbonitrile (Compound No. 362) and 8-cyclopentyl-2-((3R,4S)-3-fluoro-1-(methylsulfonyl)piperidin-4-ylamino)-7-oxo-7,8-dihydropyrido[2,3-d]pyrimidine-6-carbonitrile. (Compound No. 363)

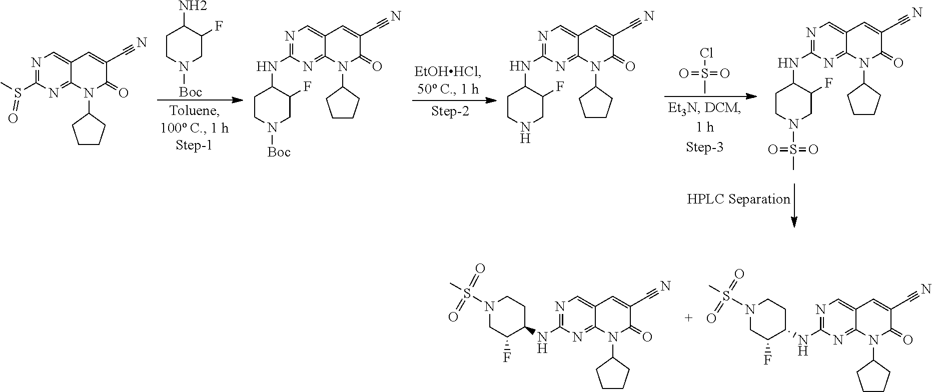

Step—1: Synthesis of tert-butyl 4-((6-cyano-8-cyclopentyl-7-oxo-7,8-dihydropyrido[2,3-d]pyrimidin-2-yl)amino)-3-fluoropiperidine-1-carboxylate: To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added tert-butyl 4-amino-3-fluoropiperidine-1-carboxylate (79 mg, 0.36 mmol, 1.1 equiv). The resultant reaction mixture was stir at 100° C. for 1 h. Progress of the reaction was monitored by LCMS. After completion of the reaction, solvent was removed under reduced pressure to obtain crude, which was used for the next step without any further purification.

Step—2: Synthesis of 8-cyclopentyl-2-((3-fluoropiperidin-4-yl)amino)-7-oxo-7,8-dihydropyrido[2,3-d]pyrimidine-6-carbonitrile: A solution of tert-butyl 4-((6-cyano-8-cyclopentyl-7-oxo-7,8-dihydropyrido[2,3-d]pyrimidin-2-yl)amino)-3-fluoropiperidine-1-carboxylate (100 mg, 0.21 mmol, 1 equiv) in 1.25 M HCl in ethanol (5 mL) was allowed to stir at 50° C. for 1 h. Progress of the reaction was monitored by LCMS. After completion of the reaction, solvent was removed under reduced pressure to obtain crude, which was used for the next step without any further purification.

Step—3: Synthesis of 8-cyclopentyl-2-((3-fluoro-1-(methylsulfonyl)piperidin-4-yl)amino)-7-oxo-7,8-dihydropyrido[2,3-d]pyrimidine-6-carbonitrile: To a stirred solution of 8-cyclopentyl-2-((3-fluoropiperidin-4-yl)amino)-7-oxo-7,8-dihydropyrido[2,3-d]pyrimidine-6-carbonitrile (80 mg, 0.2 mmol, 1 equiv) in DCM (5 mL), was added ET₃N (0.04 mL, 0.3 mmol, 1.5 equiv) and methanesulfonyl chloride (28 mg, 0.24 mmol, 1.2 equiv) at 0° C. The reaction mixture was allowed to stir at RT for 1 h. Progress of the reaction was monitored by LCMS. After completion of the reaction, the reaction mixture was diluted with water (15 mL) and extracted with DCM (50 mL). Organic layer was washed with water (50 mL), brine solution (50 mL). Organic layer was dried over anhydrous sodium sulphate and concentrated under reduced pressure to obtain crude, which was purified by reverse phase HPLC. Two peaks obtained as Peak-1 and Peak-2. LCMS: 435 [M+H]⁺, Peak-1 ¹H NMR (400 MHz, DMSO-d₆): δ8.72 (s, 1H), 8.45 (s, 1H), 8.30 (br s, 1H), 5.81 (br s, 1H), 4.79-4.67 (br s, 1H), 4.26 (br s, 1H), 3.82 (br s, 1H), 3.56 (br s, 1H), 3.24 (m, 2H), 3.03 (br s, 2H), 2.96 (s, 3H), 2.27 (br s, 2H), 1.99 (br s, 2H), 1.81 (br s, 2H), 1.64 (br s, 2H). Peak-2 ¹H NMR (400 MHz, DMSO-d₆): δ 8.72 (s, 1H), 8.45 (s, 1H), 8.30 (br s, 1H), 5.81 (br s, 1H), 4.79-4.67 (br s, 1H), 4.26 (br s, 1H), 3.82 (br s, 1H), 3.56 (br s, 1H), 3.24 (m, 2H), 3.03 (br s, 2H), 2.96 (s, 3H), 2.27 (br s, 2H), 1.99 (br s, 2H), 1.81 (br s, 2H), 1.64 (br s, 2H).

### Example—S131: Synthesis of (S)-8-cyclopentyl-2-((1-(methylsulfonyl)piperidin-3-yl)amino)-7-oxo-7,8-dihydropyrido[2,3-d]pyrimidine-6-carbonitrile (Compound No. 364)

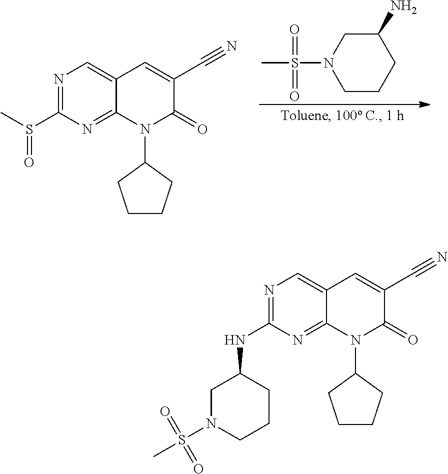

To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (3 mL), was added (S)-1-(methylsulfonyl)piperidin-3-amine (59 mg, 0.33 mmol, 1 equiv) at RT. The resultant reaction mixture was allowed to stir at 100° C. for 2H. Progress of the reaction was monitored by LCMS. After completion of the reaction, solvent was decanted off to obtain crude, which was triturated from methanol (2 mL) (crude was stirred in methanol for 1H, filtered, washed with methanol (1 mL×2) to obtain desired product. LCMS: 417 [M+H]⁺; ¹HNMR: (400 MHz, DMSO-d₆, VT @ 80° C.): δ 8.70 (s, 1H), 8.43 (s, 1H), 8.34-7.95 (m, 1H), 5.84 (p, J=8.8 Hz, 1H), 4.29-3.58 (m, 2H), 3.50 (d, J=12.0 Hz, 1H), 2.86 (s, 4H), 2.69 (t, J=8.0 Hz, 1H), 2.22 (p, J=8.7 Hz, 2H), 2.10-1.72 (m, 6H), 1.62 (p, J=10.7, 10.0 Hz, 4H).

### Example—S132: Synthesis of (R)-8-cyclopentyl-2-((1-(methylsulfonyl)piperidin-3-yl)amino)-7-oxo-7,8-dihydropyrido[2,3-d]pyrimidine-6-carbonitrile (Compound No. 365)

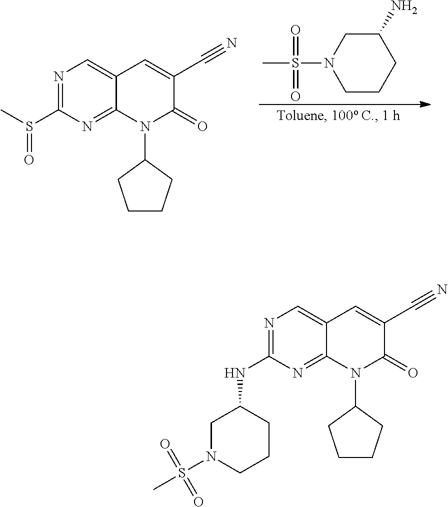

To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (3 mL), was added (R)-1-(methylsulfonyl)piperidin-3-amine (59 mg, 0.33 mmol, 1 equiv) at RT. The resultant reaction mixture was allowed to stir at 100° C. for 2H. Progress of the reaction was monitored by LCMS. After completion of the reaction, solvent was decanted off to obtain crude, which was triturated from methanol (2 mL) (crude was stirred in methanol for 1H, filtered, washed with methanol (1 mL×2)) to obtain desired product. LCMS: 417 [M+H]⁺; ¹HNMR: (400 MHz, DMSO-d₆, VT @ 80° C.): δ 8.70 (s, 1H), 8.43 (s, 1H), 8.22 (s, 1H), 5.84 (p, J=8.8 Hz, 1H), 3.91 (p, J=36.4, 30.6 Hz, 2H), 3.50 (d, J=12.0 Hz, 1H), 2.86 (s, 4H), 2.68 (q, J=8.4, 7.6 Hz, 1H), 2.22 (p, J=8.7 Hz, 2H), 2.09-1.74 (m, 6H), 1.62 (h, J=11.3, 9.5 Hz, 4H).

### Example—S133: Synthesis of 4-((6-cyano-8-cyclopentyl-7-oxo-7,8-dihydropyrido[2,3-d]pyrimidin-2-yl)amino)-N,N-dimethylpiperidine-1-sulfonamide, (Compound No. 366)

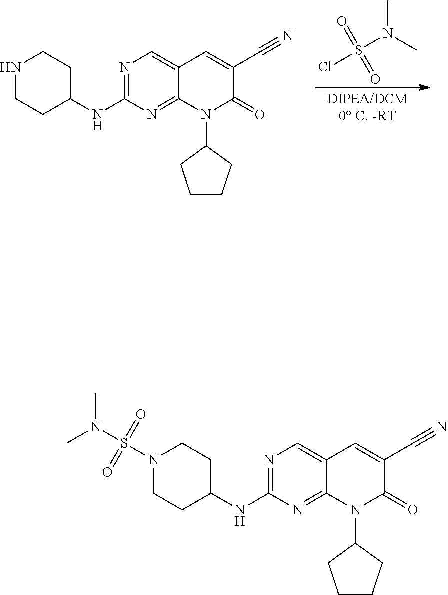

To a stirred solution of 8-cyclopentyl-7-oxo-2-(piperidin-4-ylamino)-7,8-dihydropyrido[2,3-d]pyrimidine-6-carbonitrile (100 mg, 0.3 mmol, 1 equiv) in DCM (3 mL), was added dimethylsulfamoyl chloride (42 mg, 0.33 mmol, 1 equiv) at 0° C. The resultant reaction mixture was allowed to stir at RT for 2H. Progress of the reaction was monitored by LCMS. After completion of the reaction, reaction mass concentrated, crude was triturated from methanol (2 mL) (crude was stirred in methanol for 1H, filtered, washed with methanol (1 mL×2)) to obtain desired product. LCMS: 446 [M+H]+: ¹HNMR: (400 MHz, DMSO-d₆, VT @80° C.): δ 8.69 (s, 1H), 8.41 (s, 1H), 8.37-7.96 (m, 1H), 5.84-5.74 (m, 1H), 4.05 (d, J=40.9 Hz, 1H), 3.62 (dt, J=12.7, 4.0 Hz, 2H), 3.04-2.93 (m, 2H), 2.78 (s, 6H), 2.27 (s, 3H), 1.96 (d, J=14.3 Hz, 4H), 1.84-1.75 (m, 2H), 1.65 (q, J=10.8 Hz, 4H).

### Example—S134: Synthesis of 8-cyclohexyl-2-((1-(methylsulfonyl) piperidin-4-yl) amino)-7-oxo-7, 8-dihydropyrido [2, 3-d] pyrimidine-6-carbonitrile (Compound No. 367)

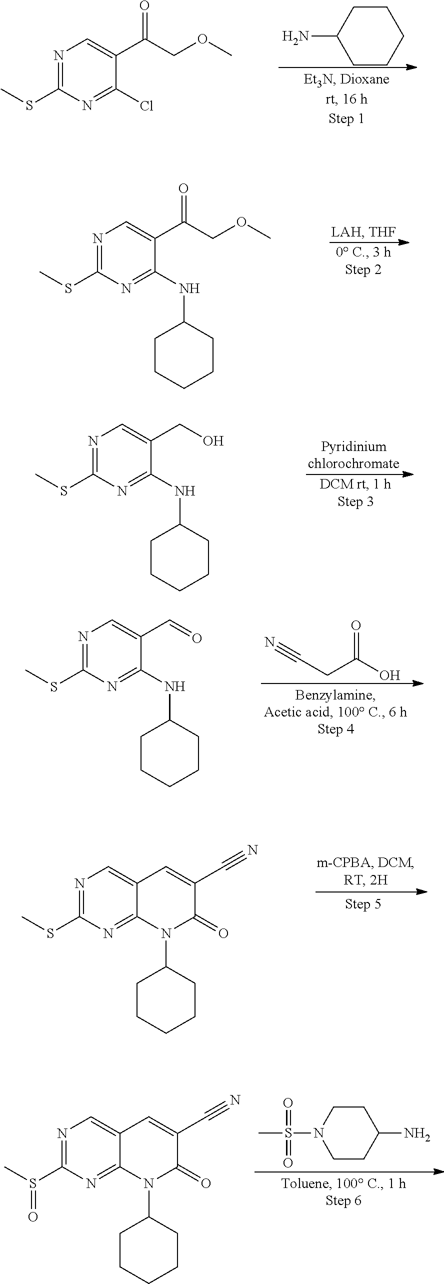

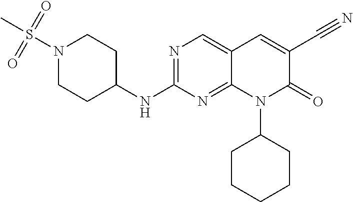 [SMILES: CS(=O)(=O)N1CCC(Nc2ncc3cc(C#N)c(=O)n(C4CCCCC4)c3n2)CC1]

Step—1: Synthesis of ethyl 4-(cyclohexylamino)-2-(methylthio)pyrimidine-5-carboxylate: To a stirred solution of ethyl 4-chloro-2-(methylthio)pyrimidine-5-carboxylate (5 g, 21.55 mmol, 1.0 equiv) in dioxane (50 mL), was added triethylamine (6.05 mL, 43.1 mmol, 2.0 equiv) and cyclohexanamine (2.34 g, 23.7 mmol, 1.1 equiv) at room temperature. Stirred the reaction mixture for 16H at room temperature. Progress of the reaction was monitored by TLC and LCMS. After completion of the reaction, the reaction mixture was diluted with water (100 mL) and extracted with ethyl acetate (100 mL×2), organic layer was washed with water (100 mL) and brine solution (100 mL). Organic layer was dried over anhydrous sodium sulphate and concentrated under reduced pressure to obtain 6 gram of desired product, which was used for the next step without any purification.

Step—2: Synthesis of (4-(cyclohexylamino)-2-(methylthio) pyrimidin-5-yl) methanol: To a stirred solution of ethyl 4-(cyclohexylamino)-2-(methylthio) pyrimidine-5-carboxylate (7 g, 24.91 mmol, 1.0 equiv) in THF (100 mL), was added portion wise LAH (2.836 g, 74.73 mmol) at 0° C. The reaction mixture was allowed to stir at room temperature for 2H. Progress of the reaction was monitored by TLC and LCMS. After completion of the reaction, the reaction mixture was quenched with saturated solution of sodium sulphate drop wise at 0° C. Obtained residue was filtered through celite bed. Filtrate was extracted with ethyl acetate (100 mL×2). The combined organic layer was washed with water (100 mL) and brine solution (100 mL). Organic layer was dried over anhydrous sodium sulphate and concentrated under reduced pressure to obtain crude, which was used for the next step without any purification.

Step—3: Synthesis of 4-(cyclohexylamino)-2-(methylthio)pyrimidine-5-carbaldehyde: To a stirred solution of (4-(cyclohexylamino)-2-(methylthio)pyrimidin-5-yl)methanol (5 g, 20.92 mmol, 1.0 equiv) in DCM (50 mL), was added pyridinium chlorochromate (8.995 g, 41.84 mmol, 2.0 equiv) at 0° C. The reaction mixture was allowed to stir at room temperature for 1 h. Progress of the reaction was monitored by TLC and LCMS. After completion of the reaction, workup done by filtration of reaction mass through celite bed and celite bed was washed by DCM (50 mL×2). Filtrate was diluted with water (100 mL), and extracted with DCM (100 mL×2). The combined organic layer was washed with sodium bicarbonate solution (100 mL) and brine solution (100 mL), Organic layer was dried over anhydrous sodium sulphate and concentrated under reduced pressure to obtain crude 4 g (80% yield), which was used for the next step without any purification.

Step—4: Synthesis of 8-cyclohexyl-2-(methylthio)-7-oxo-7,8-dihydropyrido[2,3-d]pyrimidine-6-carbonitrile: To a stirred solution of 4-(cyclohexylamino)-2-(methylthio)pyrimidine-5-carbaldehyde (5 g, 21.01 mmol, 1.0 equiv.) in Acetic acid (50 mL), was added cyano acetic acid (2.151 g, 25.31 mmol, 1.2 equiv) and Benzylamine (0.250 g, 2.109 mmol, 0.1 equiv). The reaction mixture was allowed to stir at 100° C. for 6H. Progress of the reaction was monitored by TLC and LCMS. After completion of the reaction, the reaction mixture was diluted with water (50 mL) and extracted with ethyl acetate (50 mL×2). The combined organic layer was washed with water (50×2 mL) and brine solution (50 mL). Organic layer was dried over anhydrous sodium sulphate and concentrated under reduced pressure to obtain crude compound, which was purified by normal phase combi-flash to obtain 3.5 g (58% yield) of desired product.

Step—5: Synthesis of 8-cyclohexyl-2-(methylsulfinyl)-7-oxo-7,8-dihydropyrido[2,3-d]pyrimidine-6-carbonitrile: To a stirred solution of obtain 8-cyclohexyl-2-(methylthio)-7-oxo-7,8-dihydropyrido[2,3-d]pyrimidine-6-carbonitrile (5 g, 12.95 mmol, 1 equiv) in DCM (50 mL) was added m-CPBA (77%) (3.9 g, 18.13 mmol, 1.4 equiv) at room temperature. Reaction mass was stirred at room temperature for 2 h. Progress of the reaction was monitored by TLC and LCMS. After completion of the reaction, the reaction mixture was diluted with saturated sodium bicarbonate solution (100 mL) and was extracted with DCM (100 mL×2). The combined organic layer was washed with water (100 mL) and sodium bicarbonate solution (100 mL). Organic layer was dried over anhydrous sodium sulphate and concentrated under reduced pressure to obtain crude 5 g (95% yield), which was used for the next step without any purification.

Step—6: Synthesis of 8-cyclopentyl-2-((1-(methylsulfonyl)piperidin-4-yl)amino)-7-oxo-7,8-dihydropyrido[2,3-d]pyrimidine-6-carbonitrile: To a stirred solution of 8-cyclohexyl-2-(methylsulfinyl)-7-oxo-7,8-dihydropyrido[2,3-d]pyrimidine-6-carbonitrile (100 mg, 0.33 mmol, 1 equiv) in toluene (3 mL), was added 1-(methylsulfonyl)piperidin-4-amine (2.95 g, 16.55 mmol, 1 equiv) at RT. The resultant reaction mixture was allowed to stir at 100° C. for 2H. Progress of the reaction was monitored by LCMS. After completion of the reaction, solvent was decanted off to obtain crude, which was triturated from methanol (30 mL) (crude was stirred in methanol for 1H, filtered, washed with methanol (10 mL×2) to obtain desired product. LCMS: 431 [M+H]⁺; ¹HNMR: (400 MHz, DMSO-d₆, VT @ 80° C.): δ 8.68 (d, J=5.2 Hz, 1H), 8.41 (s, 1H), 8.34 (d, J=9.5 Hz, 1H), 5.26 (t, J=11.9 Hz, 1H), 3.95 (d, J=11.5 Hz, 1H), 3.70-3.56 (m, 2H), 2.90 (d, J=6.5 Hz, 4H), 2.64-2.54 (m, 2H), 2.09 (s, 1H), 2.03 (d, J=12.1 Hz, 2H), 1.93-1.82 (m, 2H), 1.79-1.54 (m, 4H), 1.37 (q, J=12.7 Hz, 2H), 1.29-1.16 (m, 1H)

### Example—S135: Synthesis of 8-cyclobutyl-2-((1-(methylsulfonyl) piperidin-4-yl) amino)-7-oxo-7, 8-dihydropyrido [2, 3-d] pyrimidine-6-carbonitrile (Compound No. 368)

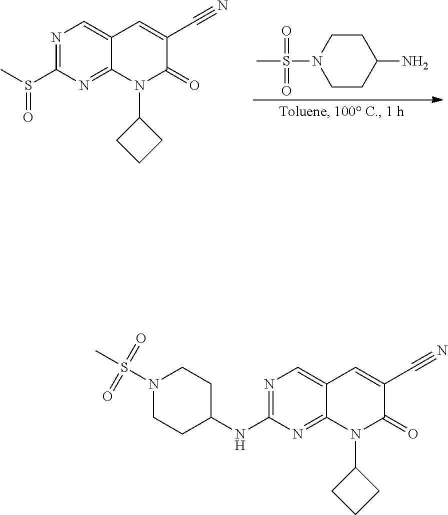 [SMILES: CS(=O)c1ncc2cc(C#N)c(=O)n(C3CCC3)c2n1 ; CS(=O)(=O)N1CCC(N)CC1 ; CS(=O)(=O)N1CCC(Nc2ncc3cc(C#N)c(=O)n(C4CCC4)c3n2)CC1]

To a stirred solution of 8-cyclobutyl-2-(methylsulfinyl)-7-oxo-7,8-dihydropyrido[2,3-d]pyrimidine-6-carbonitrile (100 mg, 0.35 mmol, 1 equiv) in toluene (50 mL), was added 1-(methylsulfonyl)piperidin-4-amine (62 mg, 0.35 mmol, 1 equiv) at RT. The resultant reaction mixture was allowed to stir at 100° C. for 2H. Progress of the reaction was monitored by LCMS. After completion of the reaction, solvent was decanted off to obtain crude, which was triturated from methanol (1 mL) (crude was stirred in methanol for 1H, filtered, washed with methanol (1 mL×2) to obtain desired product. LCMS: 403 [M+H]⁺; ¹HNMR: (400 MHz, DMSO-d₆, VT @80° C.): δ 8.67 (s, 1H), 8.41 (s, 1H), 8.23 (d, J=73.5 Hz, 1H), 5.85-5.47 (m, 1H), 4.07 (d, J=15.5 Hz, 1H), 3.72-3.56 (m, 2H), 2.91 (d, J=18.6 Hz, 5H), 2.31 (d, J=11.3 Hz, 3H), 2.12-1.76 (m, 4H), 1.70 (t, J=12.5 Hz, 2H).

### Example—S136: Synthesis of 8-(bicyclo[1.1.1]pentan-1-yl)-2-((1-(methylsulfonyl)piperidin-4-yl)amino)-7-oxo-7,8-dihydropyrido[2,3-d]pyrimidine-6-carbonitrile (Compound No. 369)

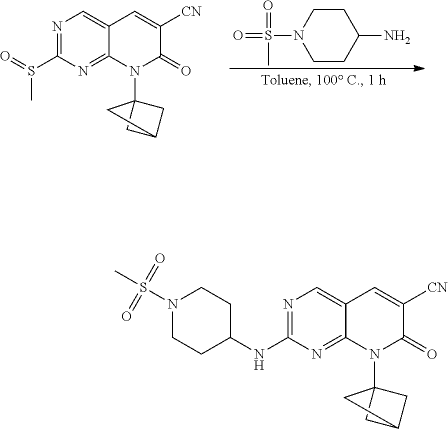 [SMILES: CS(=O)c1ncc2cc(C#N)c(=O)n(C34CC(C3)C4)c2n1 ; CS(=O)(=O)N1CCC(N)CC1 ; CS(=O)(=O)N1CCC(Nc2ncc3cc(C#N)c(=O)n(C45CC(C4)C5)c3n2)CC1]

To a stirred solution of 8-(bicyclo[1.1.1]pentan-1-yl)-2-(methylsulfinyl)-7-oxo-7,8-dihydropyrido[2,3-d]pyrimidine-6-carbonitrile (80 mg, 0.26 mmol, 1 equiv) in toluene (5 mL), was added 1-(methylsulfonyl)piperidin-4-amine (52 mg, 0.29 mmol, 1.1 equiv). The resultant reaction mixture was allowed to stir at 100° C. for 1 h. Progress of the reaction was monitored by LCMS. After completion of the reaction, solvent was removed under reduced pressure to obtain crude, which was purified by reverse phase HPLC to obtain desired product. LCMS: 415 [M+H]⁺; ¹H NMR (400 MHz, DMSO-d₆): δ 8.64 (s, 1H), 8.37 (s, 1H), 8.29 (br s, 1H), 3.97 (br s, 1H), 3.62 (d, J=12.0 Hz, 2H), 2.81-2.96 (m, 5H), 2.62 (s, 7H), 1.99-2.02 (d, J=12.0 Hz, 2H), 1.51-1.73 (m, 2H).

### Example—S137: Synthesis of 8-(bicyclo[1.1.1]pentan-1-yl)-7-oxo-2-((1,2,3,4-tetrahydroisoquinolin-6-yl)amino)-7,8-dihydropyrido[2,3-d]pyrimidine-6-carbonitrile (Compound No. 370)

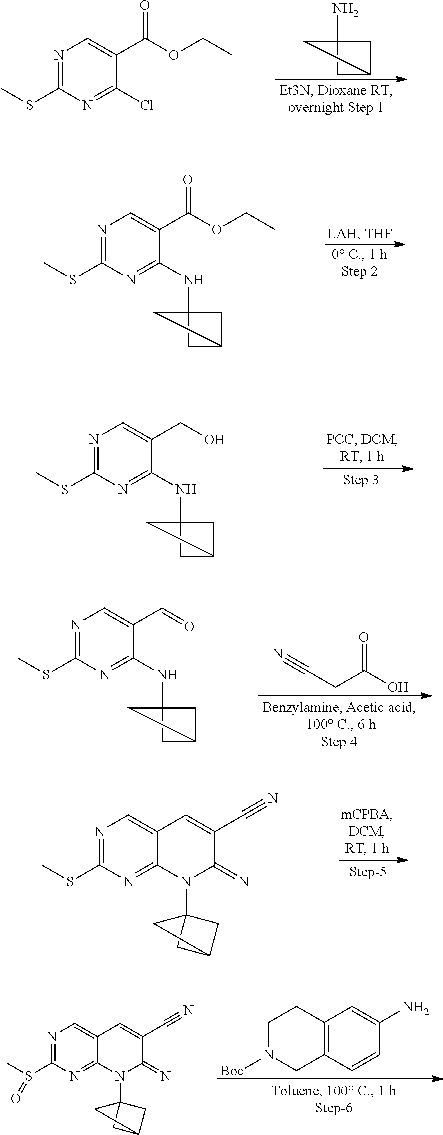

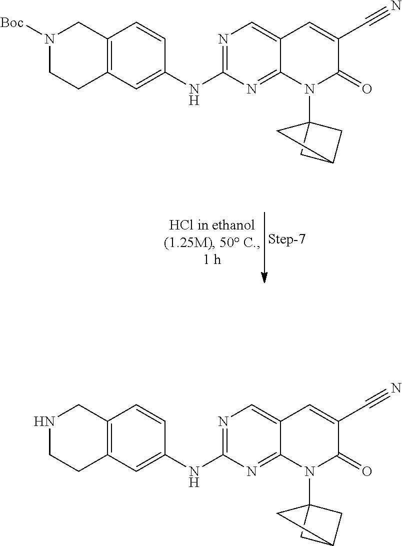 [SMILES: CC(C)(C)OC(=O)N1CCc2cc(Nc3ncc4cc(C#N)c(=O)n(C56CC(C5)C6)c4n3)ccc2C1 ; N#Cc1cc2cnc(Nc3ccc4c(c3)CCNC4)nc2n(C23CC(C2)C3)c1=O]

Step—1: Synthesis of ethyl 4-(bicyclo[1.1.1]pentan-1-ylamino)-2-(methylthio)pyrimidine-5-carboxylate: To a stirred solution of ethyl 4-chloro-2-(methylthio)pyrimidine-5-carboxylate (1000 mg, 4.29 mmol, 1 equiv) in Dioxane (10 mL), was added ET₃N (1.8 mL, 12.87 mmol, 3 equiv) and bicyclo[0.1.1]pentan-1-amine (618 mg, 5.15 mmol, 1.2 equiv) at RT. The resultant reaction mixture was allowed to stir for overnight. Progress of the reaction was monitored by TLC and LCMS. After completion of the reaction, the reaction mixture was diluted with water (50 mL) and extracted with ethyl acetate (100 mL). Organic layer was washed with water (100 mL), brine solution (100 mL). Organic layer was dried over anhydrous sodium sulphate and concentrated under reduced pressure to obtain crude, which was purified by normal phase combi flash.

Step—2: Synthesis of (4-(bicyclo[1.1.1]pentan-1-ylamino)-2-(methylthio)pyrimidin-5-yl)methanol: To a stirred solution of ethyl 4-(bicyclo[1.1.1]pentan-1-ylamino)-2-(methylthio)pyrimidine-5-carboxylate (1000 mg, 3.58 mmol, 1 equiv) in THF (15 mL), was added LAH (272 mg, 7.16 mmol, 2 equiv) at 0° C. Raise the temp. To RT and the reaction mixture was allowed to stir for 1 h. Progress of the reaction was monitored by TLC and LCMS. After completion of the reaction, the reaction mixture was quenched with saturated solution of sodium hydroxide (5 mL) at 0° C., the reaction mixture was then passes through celite bed, filtrate obtain was concentrated under reduced pressure to obtain crude, which was used for the next step without any further purification.

Step—3: Synthesis of 4-(bicyclo[1.1.1]pentan-1-ylamino)-2-(methylthio)pyrimidine-5-carbaldehyde: To a stirred solution of (4-(bicyclo [1.1.1] pentan-1-ylamino)-2-(methylthio) pyrimidin-5-yl) methanol (800 mg, 3.37 mmol, 1 equiv) in DCM (10 mL), was added PCC (729 mg, 3.37 mmol, 1 equiv) at RT. The reaction mixture was allowed to stir at RT for 1 h. Progress of the reaction was monitored by TLC and LCMS. After completion of the reaction, the reaction mixture was passes through celite bed; filtrate was concentrated under reduced pressure to obtain crude, which was used for the next step without any further purification.

Step—4: Synthesis of 8-(bicyclo[1.1.1]pentan-1-yl)-2-(methylthio)-7-oxo-7,8-dihydropyrido[2,3-d]pyrimidine-6-carbonitrile: To a stirred solution of 4-(bicyclo[1.1.1]pentan-1-ylamino)-2-(methylthio)pyrimidine-5-carbaldehyde (400 mg, 1.7 mmol, 1 equiv) in Acetic acid (5 mL), was added Cyanoacetic acid (217 mg, 2.5 mmol, 1.5 equiv) and Benzyl amine (0.02 mL, 0.17 mmol, 0.1 equiv). The reaction mixture was allowed to stir at 100° C. for 6 h. Progress of the reaction was monitored by LCMS. After completion of the reaction, the reaction mixture was diluted with water (50 mL), solid observed in the reaction mixture was filtered and dried under vacuum to obtain solid crude compound, which was used for the next step without any further purification.

Step—5: Synthesis of 8-(bicyclo[1.1.1]pentan-1-yl)-2-(methylsulfinyl)-7-oxo-7,8-dihydropyrido[2,3-d]pyrimidine-6-carbonitrile: To a stirred solution of 8-(bicyclo[1.1.1]pentan-1-yl)-2-(methylthio)-7-oxo-7,8-dihydropyrido[2,3-d]pyrimidine-6-carbonitrile (200 mg, 0.7 mmol, 1 equiv) in DCM (5 mL), was added m-CPBA (158 mg, 0.91 mmol, 1.3 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain crude, which was used for the next step without any further purification.

Step—6: Synthesis of tert-butyl 6-((8-(bicyclo[1.1.1]pentan-1-yl)-6-cyano-7-oxo-7,8-dihydropyrido[2,3-d]pyrimidin-2-yl)amino)-3,4-dihydroisoquinoline-2(1H)-carboxylate: To a stirred solution of 8-(bicyclo[1.1.1]pentan-1-yl)-2-(methylsulfinyl)-7-oxo-7,8-dihydropyrido[2,3-d]pyrimidine-6-carbonitrile (80 mg, 0.26 mmol, 1 equiv) in toluene (5 mL), was added tert-butyl 6-amino-3,4-dihydroisoquinoline-2(1H)-carboxylate (73 mg, 0.29 mmol, 1.1 equiv). The resultant reaction mixture was allowed to stir at 100° C. for 1 h. Progress of the reaction was monitored by LCMS. After completion of the reaction, solvent was removed under reduced pressure to obtain crude, which was used for the next step without any further purification.

Step—7: Synthesis of 8-(bicyclo[1.1.1]pentan-1-yl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((8-(bicyclo[1.1.1]pentan-1-yl)-6-cyano-7-oxo-7,8-dihydropyrido[2,3-d]pyrimidin-2-yl)amino)-3,4-dihydroisoquinoline-2(1H)-carboxylate (100 mg, 0.2 mmol, 1 equiv) in 1.25 M HCl in ethanol (5 mL) was allowed to stir at 50° C. for overnight. Progress of the reaction was monitored by LCMS. After completion of the reaction, solvent was removed under reduced pressure and the residue was dried under Lyophiliser to obtain crude compound, which was purified by trituration with methanol to obtain desired product. LCMS: 385 [M+H], ¹H NMR (400 MHz, Methanol-d4): δ 8.72 (s, 1H), 8.32 (s, 1H), 7.57 (br s, 1H), 7.50 (br s, 1H), 7.25 (d, J=7.9 Hz, 1H), 4.37 (s, 2H), 3.53 (t, J=6.4 Hz, 2H), 3.15 (t, J=6.1 Hz, 2H), 2.62 (br s, 3H), 2.55 (br s, 2H), 1.19-1.38 (m, 2H).

### Example—S138: Synthesis of 8-cyclopentyl-7-oxo-2-((1-((trifluoromethyl)sulfonyl)piperidin-4-yl)amino)-7,8-dihydropyrido[2,3-d]pyrimidine-6-carbonitrile (Compound No. 371)

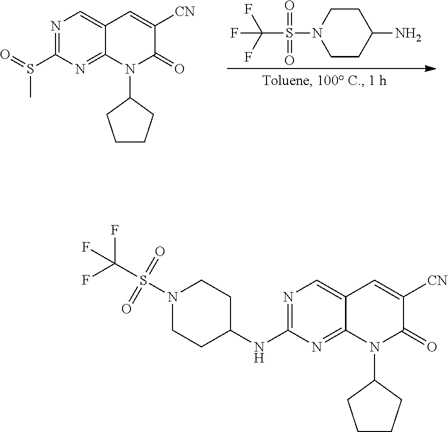 [SMILES: CS(=O)c1ncc2cc(C#N)c(=O)n(C3CCCC3)c2n1 ; NC1CCN(S(=O)(=O)C(F)(F)F)CC1 ; N#Cc1cc2cnc(NC3CCN(S(=O)(=O)C(F)(F)F)CC3)nc2n(C2CCCC2)c1=O]

To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 1-((trifluoromethyl)sulfonyl)piperidin-4-amine (85 mg, 0.36 mmol, 1.1 equiv). The resultant reaction mixture was allowed to stir at 100° C. for 1 h. Progress of the reaction was monitored by LCMS. After completion of the reaction, solvent was removed under reduced pressure to obtain crude, which was purified by trituration with methanol to obtain desired product. LCMS: 471 [M+H]⁺, ¹H NMR (400 MHz, DMSO-d₆): δ 8.71 (s, 1H), 8.43 (s, 1H), 8.28 (br s, 1H), 5.81 (br s, 1H), 4.14 (br s, 1H), 3.84 (s, 2H), 3.38 (t, J=11.6 Hz, 2H), 2.31-2.25 (br s, 2H), 2.06-2.03 (br s, 4H), 1.81 (br s, 2H), 1.54-1.75 (m, 4H).

### Example—S139: Synthesis of 8-cyclopentyl-2-((1-(methylsulfonyl)azetidin-3-yl)amino)-7-oxo-7,8-dihydropyrido[2,3-d]pyrimidine-6-carbonitrile (Compound No. 372)

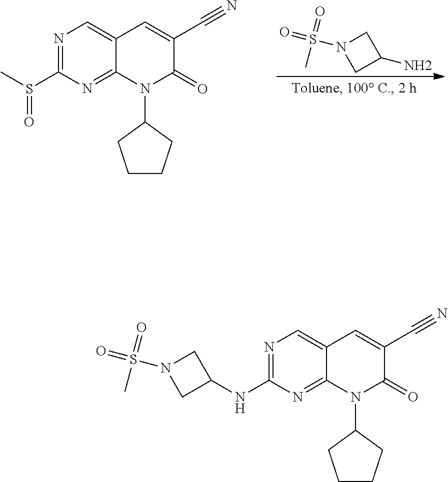

To a stirred solution of 8-cyclopentyl-2-(methylsulfinyl)-7-oxo-7,8-dihydropyrido[2,3-d]pyrimidine-6-carbonitrile (0.3 g, 0.99 mmol, 1 equiv) in tolune (5 mL), was added 1-(methylsulfonyl)azetidin-3-amine (0.18 g, 1.1 mmol, 1.1 eq) at RT. The resultant reaction mixture was allowed to stir for 2H at 100° C. Progress of the reaction was monitored by LCMS. After completion of the reaction, solvent was decanted off to obtain crude, which was triturated from methanol (2 mL) (crude was stirred in methanol for 1H, filtered, washed with methanol (5 mL×2)) to obtain desired product as an off white solid LCMS: 403 [M+H]⁺; ¹H NMR: (400 MHz, DMSO-d₆): δ 8.88 (br s, 1H) 8.67-8.80 (m, 1H) 8.52 (s, 1H) 5.83 (br s, 1H) 4.68 (d, J=6.5 Hz, 1H) 4.04-4.18 (m, 2H) 3.94 (t, J=7.2 Hz, 1H) 3.05 (s, 3H) 2.18 (br s, 2H) 1.96 (br s, 3H) 1.79 (br s, 2H) 1.67 (br s, 3H)

### Example—S140: Synthesis of 8-cyclopentyl-2-((1-((1-methyl-1H-pyrazol-3-yl)sulfonyl)piperidin-4-yl)amino)-7-oxo-7,8-dihydropyrido[2,3-d]pyrimidine-6-carbonitrile (Compound No. 373)

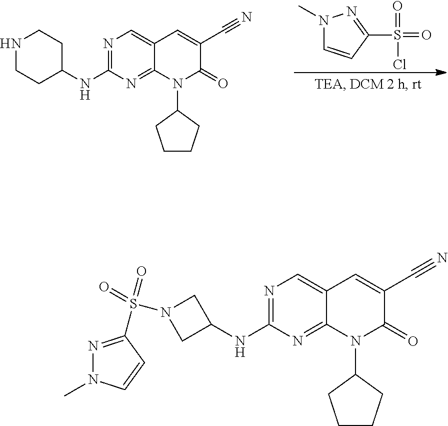

To a stirred solution of 8-cyclopentyl-7-oxo-2-(piperidin-4-ylamino)-7,8-dihydropyrido[2,3-d]pyrimidine-6-carbonitrile (0.3 g, 0.9 mmol, 1 equiv) in DCM (5 mL), was added triethylamine dropwise (0.22 mL, 1.2 mmol, 1.2 eq) at RT. To this was added 1-methyl-1H-pyrazole-3-sulfonyl chloride (0.159 g, 1.0 mmol, 1 eq), the resultant reaction mixture was allowed to stir for 2H. Progress of the reaction was monitored by LCMS. After completion of the reaction, the reaction mixture was diluted with water (10 mL) and was extracted with DCM (10 mL×2). Organic layer was dried over anhydrous sodium sulphate and concentrated under reduced pressure to obtain crude, the crude which was triturated from methanol (30 mL) (crude was stirred in methanol for 1H, filtered, washed with methanol (2 mL×2)) to obtain desired product as an off white LCMS: 483.5 [M+H]⁺; ¹H NMR: (400 MHz, DMSO-d₆): δ 8.46 (s, 1H) 8.34 (d, J=8.3 Hz, 1H) 7.82-8.04 (m, 1H) 6.57-6.76 (m, 1H) 5.78-5.93 (m, 1H) 3.95 (s, 3H) 3.81 (br s, 1H) 3.62 (d, J=11.4 Hz, 2H) 2.51-2.79 (m, 3H) 2.26 (br s, 2H) 1.94 (m, J=10.1 Hz, 10H)

### Example—S141: Synthesis of 8-cyclopentyl-2-((1-((2-methoxyethyl)sulfonyl)piperidin-4-yl)amino)-7-oxo-7,8-dihydropyrido[2,3-d]pyrimidine-6-carbonitrile (Compound No. 374)

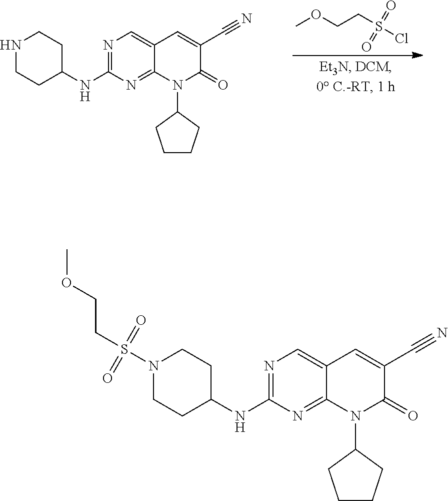

To a stirred solution of 8-cyclopentyl-7-oxo-2-(piperidin-4-ylamino)-7,8-dihydropyrido[2,3-d]pyrimidine-6-carbonitrile (100 mg, 0.26 mmol, 1 equiv) in DCM (5 mL), was added ET₃N (0.1 mL, 0.52 mmol, 2 equiv) and 2-methoxyethane-1-sulfonyl chloride (51 mg, 0.32 mmol, 1.2 equiv) at 0° C. Raise the temp. to RT and the reaction mixture was allowed to stir for 1 h. Progress of the reaction was monitored by LCMS. After completion of the reaction, the reaction mixture was diluted with water (15 mL) and extracted with DCM (50 mL). Organic layer was washed with water (50 mL), brine solution (50 mL). Organic layer was dried over anhydrous sodium sulphate and concentrated under reduced pressure to obtain crude, which was purified by reverse phase HPLC to obtain desired product. LCMS: 461 [M+H]⁺, ¹H NMR (400 MHz, DMSO-d₆): δ 8.69 (s, 1H), 8.42 (s, 1H), 8.32 (br s, 1H), 5.79 (br s, 1H), 3.97 (br s, 1H), 3.56-3.72 (m, 4H), 3.12 (br s, 4H), 2.96 (t, J=11.2 Hz, 2H), 2.33 (br s, 2H), 1.99 (br s, 4H), 1.80 (br s, 2H), 1.43-1.72 (m, 5H).

### Example—S142: Synthesis of 8-(2,6-difluorophenyl)-7-oxo-2-((1,2,3,4-tetrahydroisoquinolin-6-yl)amino)-7,8-dihydropyrido[2,3-d]pyrimidine-6-carbonitrile (Compound No. 375)

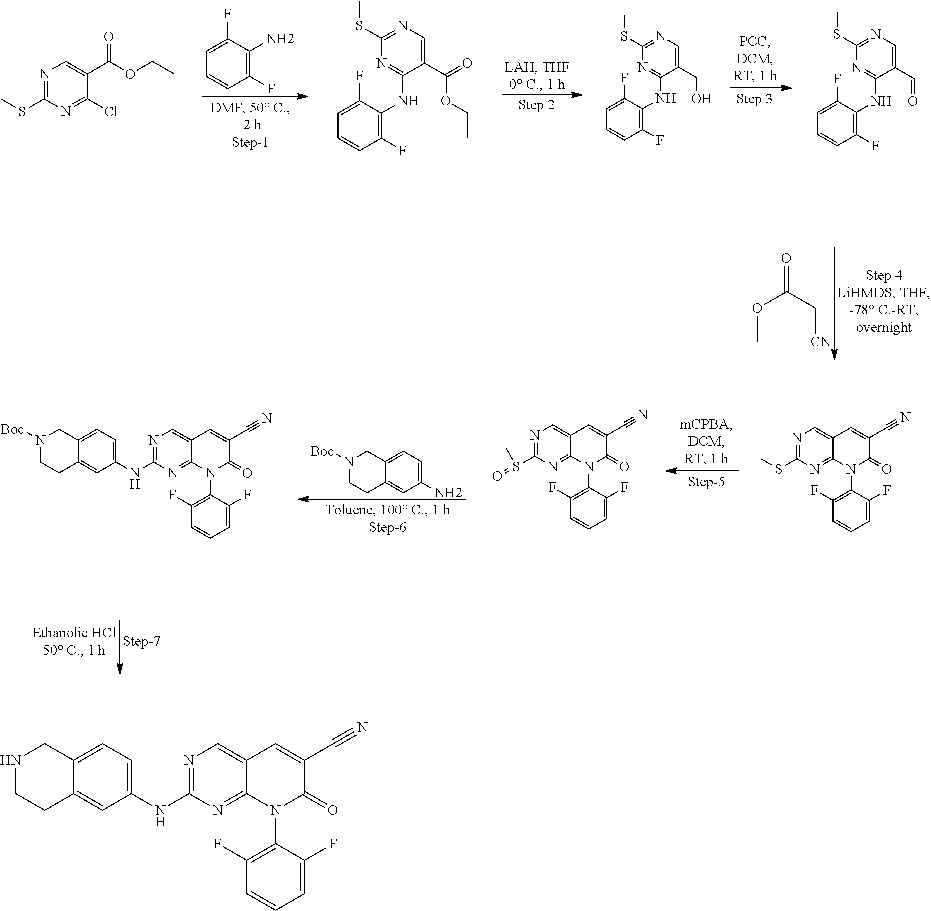

Step—1: Synthesis of ethyl 4-((2,6-difluorophenyl)amino)-2-(methylthio)pyrimidine-5-carboxylate: To a stirred solution of ethyl 4-chloro-2-(methylthio)pyrimidine-5-carboxylate (3000 mg, 12.9 mmol, 1 equiv) in DMF (20 mL), was added 2,6-difluoroaniline (3336 mg, 25.86 mmol, 1.2 equiv) at RT. The resultant reaction mixture was allowed to stir for 2 h at 50° C. Progress of the reaction was monitored by TLC and LCMS. After completion of the reaction, the reaction mixture was diluted with methanol (10 mL) and water (100 mL). Solid observed in the reaction mixture was filtered and dried under vacuum to obtain crude, which was used for the next step without any further purification.

Step—2: Synthesis of (4-((2,6-difluorophenyl)amino)-2-(methylthio)pyrimidin-5-yl)methanol: To a stirred solution of ethyl 4-((2,6-difluorophenyl)amino)-2-(methylthio)pyrimidine-5-carboxylate (3000 mg, 9.23 mmol, 1 equiv) in THF (30 mL), was added LAH (702 mg, 18.46 mmol, 2 equiv) at 0° C. Raise the temp. To RT and the reaction mixture was allowed to stir for 1 h. Progress of the reaction was monitored by TLC and LCMS. After completion of the reaction, the reaction mixture was quenched with saturated solution of sodium hydroxide (5 mL) at 0° C., the reaction mixture was then passes through celite bed, filtrate obtain was concentrated under reduced pressure to obtain crude, which was used for the next step without any further purification.

Step—3: Synthesis of 4-((2,6-difluorophenyl)amino)-2-(methylthio)pyrimidine-5-carbaldehyde: To a stirred solution of (4-((2,6-difluorophenyl)amino)-2-(methylthio)pyrimidin-5-yl)methanol (1500 mg, 5.3 mmol, 1 equiv) in DCM (15 mL), was added PCC (1145 mg, 5.3 mmol, 1 equiv) at RT. The reaction mixture was allowed to stir at RT for 1 h. Progress of the reaction was monitored by TLC and LCMS. After completion of the reaction, the reaction mixture was passes through celite bed; filtrate was concentrated under reduced pressure to obtain crude, which was purified by normal phase combi flash to obtain desired product.

Step—4: Synthesis of 8-(2,6-difluorophenyl)-2-(methylthio)-7-oxo-7,8-dihydropyrido[2,3-d]pyrimidine-6-carbonitrile: To a stirred solution of 4-((2,6-difluorophenyl)amino)-2-(methylthio)pyrimidine-5-carbaldehyde (300 mg, 1.06 mmol, 1 equiv) in THF (5 mL), was added Cyanoacetic acid (211 mg, 2.13 mmol, 2 equiv) at −78° C. The reaction mixture was allowed to stir for 15 min. at same temperature, followed by the addition of LiHMDS (4 mL, 4.24 mmol, 4 equiv). The reaction mixture was allowed to stir for 30 min. at −78° C. Raise the temperature to RT and the reaction mixture was the allowed to stir for overnight. Progress of the reaction was monitored by LCMS. After completion of the reaction, the reaction mixture was diluted with water (50 mL) and extracted with ethyl acetate (100 mL). Organic layer was washed with water (100 mL), brine solution (100 mL). Organic layer was dried over anhydrous sodium sulphate and concentrated under reduced pressure to obtain crude, which was purified by normal phase combi flash to obtain desired product.

Step—5: Synthesis of 8-(2,6-difluorophenyl)-2-(methylsulfinyl)-7-oxo-7,8-dihydropyrido[2,3-d]pyrimidine-6-carbonitrile: To a stirred solution of 8-(2,6-difluorophenyl)-2-(methylthio)-7-oxo-7,8-dihydropyrido[2,3-d]pyrimidine-6-carbonitrile (100 mg, 0.3 mmol, 1 equiv) in DCM (3 mL), was added m-CPBA (68 mg, 0.39 mmol, 1.3 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain crude, which was used for the next step without any further purification.

Step—6: Synthesis of tert-butyl 6-((6-cyano-8-(2,6-difluorophenyl)-7-oxo-7,8-dihydropyrido[2,3-d]pyrimidin-2-yl)amino)-3,4-dihydroisoquinoline-2(1H)-carboxylate: To a stirred solution of 8-(2,6-difluorophenyl)-2-(methylsulfinyl)-7-oxo-7,8-dihydropyrido[2,3-d]pyrimidine-6-carbonitrile (50 mg, 0.14 mmol, 1 equiv) in toluene (2 mL), was added tert-butyl 6-amino-3,4-dihydroisoquinoline-2(1H)-carboxylate (43 mg, 0.17 mmol, 1.2 equiv). The resultant reaction mixture was allowed to stir at 100° C. for 1 h. Progress of the reaction was monitored by LCMS. After completion of the reaction, solvent was removed under reduced pressure to obtain crude, which was used for the next step without any further purification.

Step—7: Synthesis of 8-(2,6-difluorophenyl)-7-oxo-2-((1,2,3,4-tetrahydroisoquinolin-6-yl)amino)-7,8-dihydropyrido[2,3-d]pyrimidine-6-carbonitrile: A solution of tert-butyl 6-((6-cyano-8-(2,6-difluorophenyl)-7-oxo-7,8-dihydropyrido[2,3-d]pyrimidin-2-yl)amino)-3,4-dihydroisoquinoline-2(1H)-carboxylate (70 mg, 0.13 mmol, 1 equiv) in 1.25 M HCl in ethanol (5 mL) was allowed to stir at 50° C. for 1 h. Progress of the reaction was monitored by LCMS. After completion of the reaction, solvent was removed under reduced pressure and the residue was dried under Lyophiliser to obtain crude compound, which was purified by reverse phase HPLC to obtain desired product. LCMS: 431 [M+H], ¹H NMR (METHANOL-d₄, 400 MHz): δ 8.86 (s, 1H), 8.61 (s, 1H), 7.72 (br s, 1H), 7.31 (t, J=8.3 Hz, 2H), 7.14 (br s, 2H), 6.85 (br s, 1H), 4.03 (br s, 2H), 3.20 (br s, 2H), 2.69 (br s, 2H).

### Example—S143: Synthesis of 8-(2,6-difluorophenyl)-2-((1-(methylsulfonyl)piperidin-4-yl)amino)-7-oxo-7,8-dihydropyrido[2,3-d]pyrimidine-6-carbonitrile (Compound No. 376)

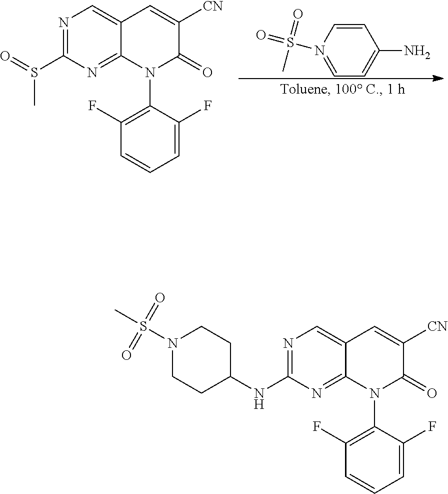

To a stirred solution of 8-(2,6-difluorophenyl)-2-(methylsulfinyl)-7-oxo-7,8-dihydropyrido[2,3-d]pyrimidine-6-carbonitrile (50 mg, 0.14 mmol, 1 equiv) in toluene (2 mL), was added 1-(methylsulfonyl)piperidin-4-amine (30 mg, 0.17 mmol, 1.1 equiv). The resultant reaction mixture was allowed to stir at 100° C. for 1 h. Progress of the reaction was monitored by LCMS. After completion of the reaction, solvent was removed under reduced pressure to obtain crude, which was purified by reverse phase HPLC to obtain desired product. LCMS: 461 [M+H]+, ¹H NMR (400 MHz, DMSO-d₆): δ 8.81 (br s, 1H), 8.71 (s, 1H), 8.58 (br s, 1H), 7.69 (br s, 1H), 7.36 (t, J=8.1 Hz, 2H), 4.15 (br s, 1H), 3.51 (br s, 2H), 2.84 (s, 3H), 1.88 (br s, 1H), 1.77 (br s, 1H), 1.66 (br s, 2H), 1.49 (br s, 2H).

### Example—S144: Synthesis of 4-((6-cyano-8-cyclopentyl-7-oxo-7,8-dihydropyrido[2,3-d]pyrimidin-2-yl)amino)benzenesulfonamide (Compound No. 377)

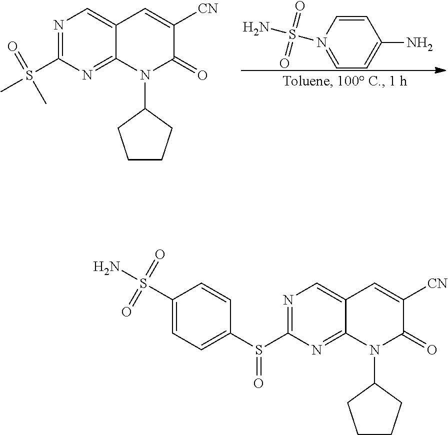

To a stirred solution of 8-cyclopentyl-2-(methylsulfinyl)-7-oxo-7,8-dihydropyrido[2,3-d]pyrimidine-6-carbonitrile (100 mg, 0.33 mmol, 1 equiv) in toluene (5 mL), was added 4-aminobenzenesulfonamide (62 mg, 0.36 mmol, 1.1 equiv). The resultant reaction mixture was allowed to stir at 100° C. for 1 h. Progress of the reaction was monitored by LCMS. After completion of the reaction, solvent was removed under reduced pressure to obtain crude, which was purified by crystallization with IPA to obtain desired product. LCMS: 411 [M+H]⁺, ¹H NMR (400 MHz, DMSO-d₆): δ 10.82 (br s, 1H), 8.92 (s, 1H), 8.64 (s, 1H), 7.90 (d, J=8.8 Hz, 2H), 7.81 (d, J=8.8 Hz, 2H), 7.29 (s, 2H), 5.85 (br s, 1H), 2.20 (d, J=7.9 Hz, 2H), 1.96 (br s, 2H), 1.85 (br s, 2H), 1.63 (d, J=4.8 Hz, 2H).

### Example—S145: Synthesis of 8-cyclopentyl-2-((1-((6-hydroxyhexyl)sulfonyl)piperidin-4-yl)amino)-7-oxo-7,8-dihydropyrido[2,3-d]pyrimidine-6-carbonitrile (Compound No. 378)

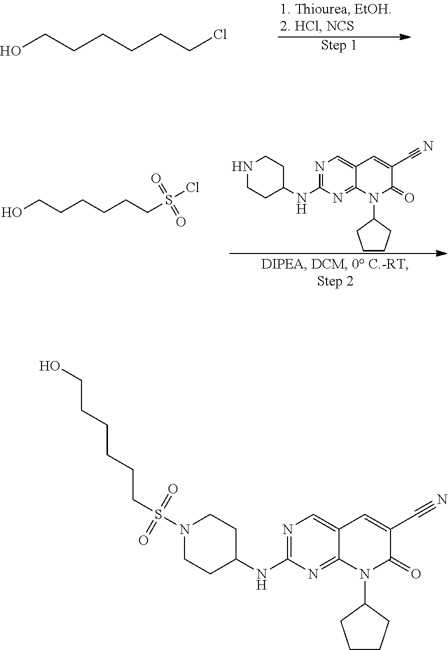 [SMILES: OCCCCCCCl ; O=S(=O)(Cl)CCCCCCO ; N#Cc1cc2cnc(NC3CCNCC3)nc2n(C2CCCC2)c1=O ; N#Cc1cc2cnc(NC3CCN(S(=O)(=O)CCCCCCO)CC3)nc2n(C2CCCC2)c1=O]

Step—1: Synthesis of 6-hydroxyhexane-1-sulfonyl chloride: To a stirred solution of 6-chlorohexan-1-ol (3000 mg, 22.05 mmol, 1 equiv) in ethanol (30 mL), was added thiourea (1676 mg, 22.05 mmol, 1 equiv). The resultant reaction mixture was allowed to stir for 1 h at 100° C. Remove the EtOH at reduced pressure. Add the obtained sticky oil slowly to a mixture of NCS (5909 mg, 44 mmol, 2 equiv), 2M HCl (15 mL) and MeCN (30 mL) in a 10° C. water bath to maintain the internal temperature between 10° C. The resultant reaction mixture was allowed to stir for 1 h at RT. Progress of the reaction was monitored by NMR. After completion of the reaction, the reaction mixture was diluted with water (100 mL) and extracted with ethyl acetate (150 mL×2). Organic layer was washed with water (150 mL), brine solution (150 mL). Organic layer was dried over anhydrous sodium sulphate and concentrated under reduced pressure to obtain crude, which was used for the next step without any further purification.

Step—2: Synthesis of 8-cyclopentyl-2-((1-((6-hydroxyhexyl)sulfonyl)piperidin-4-yl)amino)-7-oxo-7,8-dihydropyrido[2,3-d]pyrimidine-6-carbonitrile: To a stirred solution of 8-cyclopentyl-7-oxo-2-(piperidin-4-ylamino)-7,8-dihydropyrido[2,3-d]pyrimidine-6-carbonitrile (200 mg, 0.53 mmol, 1 equiv) in DCM (5 mL), was added DIPEA (0.3 mL, 1.59 mmol, 3 equiv) and 6-hydroxyhexane-1-sulfonyl chloride (536 mg, 2.68 mmol, 5 equiv) at 0° C. Raise the temp. to RT and the reaction mixture was allowed to stir for 1 h. Progress of the reaction was monitored by LCMS. After completion of the reaction, the reaction mixture was diluted with water (30 mL) and extracted with DCM (100 mL). Organic layer was washed with water (100 mL), brine solution (100 mL). Organic layer was dried over anhydrous sodium sulphate and concentrated under reduced pressure to obtain crude, which was purified by reverse phase HPLC to obtain desired product. LCMS: 503[M+H]⁺, ¹H NMR (400 MHz, DMSO-d₆): δ 8.69 (s, 1H), 8.42 (s, 1H), 8.33 (br s, 1H), 5.79 (br s, 1H), 4.10-3.98 (br s, 2H), 3.64 (s, 2H), 3.42 (t, J=6.4 Hz, 2H), 2.93-3.22 (m, 4H), 2.33-2.27 (br s, 2H), 1.99 (br s, 4H), 1.80 (br s, 2H), 1.75-1.52 (m, 6H), 1.22-1.52 (m, 6H).

### Example—S146: Synthesis of 8-cyclopentyl-6-cyclopropyl-2-((1-(methylsulfonyl)piperidin-4-yl)amino)pyrido[2,3-d]pyrimidin-7(8H)-one (Compound No. 379)

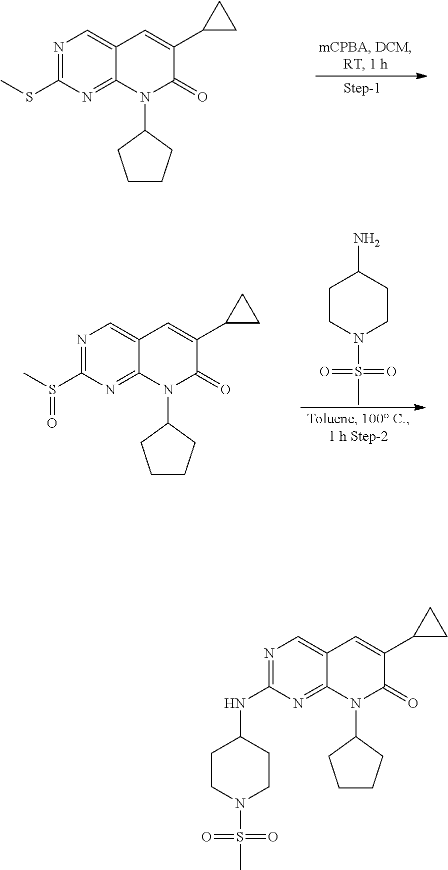 [SMILES: CSc1ncc2cc(C3CC3)c(=O)n(C3CCCC3)c2n1 ; CS(=O)c1ncc2cc(C3CC3)c(=O)n(C3CCCC3)c2n1 ; CS(=O)(=O)N1CCC(N)CC1 ; CS(=O)(=O)N1CCC(Nc2ncc3cc(C4CC4)c(=O)n(C4CCCC4)c3n2)CC1]

Step—1: Synthesis of 8-cyclopentyl-6-cyclopropyl-2-(methylsulfinyl)pyrido[2,3-d]pyrimidin-7(8H)-one: To a stirred solution of 8-cyclopentyl-6-cyclopropyl-2-(methylthio)pyrido [2,3-d]pyrimidin-7(8H)-one (500 mg, 1.66 mmol, 1 equiv) in DCM (5 mL), was added m-CPBA (414 mg, 2.32 mmol, 1.4 equiv) at RT. Then the reaction mixture was allowed to stir for 1 h. Progress of the reaction was monitored by LCMS. After completion of the reaction, the reaction mixture was diluted with DCM (50 mL) and washed with water (50 mL), brine solution (50 mL). Organic layer was dried over anhydrous sodium sulphate and concentrated under reduced pressure to obtain crude, which was used for the next step without any further purification.

Step—2: Synthesis of 8-cyclopentyl-6-cyclopropyl-2-((1-(methylsulfonyl)piperidin-4-yl)amino)pyrido[2,3-d]pyrimidin-7(8H)-one: To a stirred solution of 8-cyclopentyl-6-cyclopropyl-2-(methylsulfinyl)pyrido[2,3-d]pyrimidin-7(8H)-one (300 mg, 0.94 mmol, 1 equiv) in toluene (5 mL), was added tert-butyl 6-amino-3,4-dihydroisoquinoline-2(1H)-carboxylate (202 mg, 1.13 mmol, 1.2 equiv). The resultant reaction mixture was allowed to stir at 100° C. for 1 h. Progress of the reaction was monitored by LCMS. After completion of the reaction, solvent was removed under reduced pressure to obtain crude, which was purified by trituration with methanol. LCMS: 432 [M+H]⁺, ¹H NMR (400 MHz, DMSO-d₆): δ 8.48 (s, 1H), 7.37 (br s, 1H), 7.23 (s, 1H), 5.73-5.95 (m, 1H), 3.95 (br s, 1H), 3.62 (s, 2H), 2.72-2.95 (m, 5H), 2.33 (br s, 2H), 2.01 (br s, 5H), 1.76 (br s, 2H), 1.65 (br s, 4H), 0.86 (d, J=7.5 Hz, 2H), 0.63 (d, J=4.4 Hz, 2H).

### Example—S147: Synthesis of 8-((1R,2R)-2-hydroxy-2-methylcyclopentyl)-2-(1-(methylsulfonyl)piperidin-4-ylamino)-7-oxo-7,8-dihydropyrido[2,3-d]pyrimidine-6-carbonitrile (Compound No. 380)

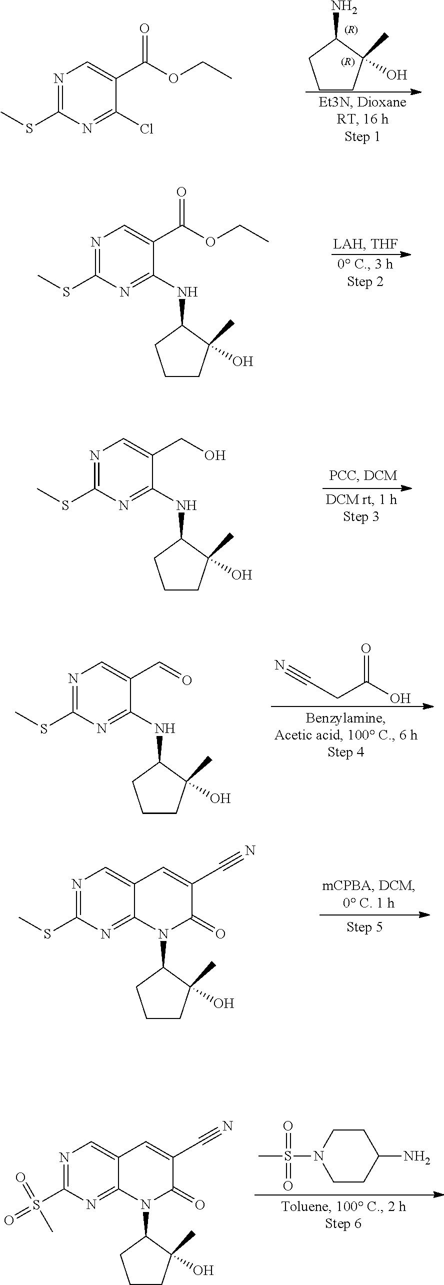

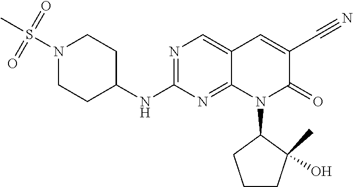 [SMILES: C[C@@]1(O)CCC[C@H]1n1c(=O)c(C#N)cc2cnc(NC3CCN(S(C)(=O)=O)CC3)nc21]

Step—1: Synthesis of ethyl 4-((1R,2R)-2-hydroxy-2-methylcyclopentylamino)-2-(methylthio)pyrimidine-5-carboxylate: To a stirred solution of ethyl 4-chloro-2-(methylthio)pyrimidine-5-carboxylate (5000 mg, 21.55 mmol, 1 equiv) in Dioxane (50 mL), was added ET₃N (4.5 mL, 32.32 mmol, 1.5 equiv) and (1R,2R)-2-amino-1-methylcyclopentanol (2974 mg, 25.86 mmol, 1.2 equiv) at RT. The resultant reaction mixture was allowed to stir for 16H at RT. Progress of the reaction was monitored by TLC and LCMS. After completion of the reaction, the reaction mixture was diluted with water (100 mL) and extracted with ethyl acetate (150 mL×2). Organic layer was washed with water (150 mL), brine solution (150 mL). Organic layer was dried over anhydrous sodium sulphate and concentrated under reduced pressure to obtain crude, which was used for the next step without any further purification.

Step—2: Synthesis of (1R,2R)-2-(5-(hydroxymethyl)-2-(methylthio)pyrimidin-4-ylamino)-1-methylcyclopentanol: To a stirred solution of ethyl 4-((1R,2R)-2-hydroxy-2-methylcyclopentylamino)-2-(methylthio)pyrimidine-5-carboxylate (6000 mg, 19.29 mmol, 1 equiv) in THF (60 mL), was added LAH (1466 mg, 38.5 mmol, 2 equiv) at 0° C. Raised the temperature to RT and the reaction mixture was allowed to stir for 2 h. Progress of the reaction was monitored by TLC and LCMS. After completion of the reaction, the reaction mixture was quenched with saturated solution of sodium hydroxide (5 mL) at 0° C., the reaction mixture was then passes through celite bed, filtrate obtain was concentrated under reduced pressure to obtain crude, which was used for the next step without any further purification.

Step—3: Synthesis of 4-((1R,2R)-2-hydroxy-2-methylcyclopentylamino)-2-(methylthio)pyrimidine-5-carbaldehyde: To a stirred solution of (1R,2R)-2-(5-(hydroxymethyl)-2-(methylthio)pyrimidin-4-ylamino)-1-methylcyclopentanol (4500 mg, 16.72 mmol, 1 equiv) in DCM (50 mL), was added PCC (3613 mg, 16.72 mmol, 1 equiv) at RT. The reaction mixture was allowed to stir at RT for 1 h. Progress of the reaction was monitored by TLC and LCMS. After completion of the reaction, the reaction mixture was passes through celite bed; filtrate was concentrated under reduced pressure to obtain crude, which was purified by normal phase combi flash to obtain desired product.

Step—4: Synthesis of 8-((1R,2R)-2-hydroxy-2-methylcyclopentyl)-2-(methylthio)-7-oxo-7,8-dihydropyrido[2,3-d]pyrimidine-6-carbonitrile: To a stirred solution of 4-((1R,2R)-2-hydroxy-2-methylcyclopentylamino)-2-(methylthio)pyrimidine-5-carbaldehyde (1.0 g, 3.74 mmol, 1.0 equiv.) in Acetic acid (5 mL), was added acetic acid (0.636 g, 7.48 mmol, 2 equiv) and Benzylamine (0.040 g, 0.074 mmol, 0.1 equiv). The reaction mixture was allowed to stir at 100° C. for 6H. Progress of the reaction was monitored by TLC and LCMS. After completion of the reaction, the reaction mixture was diluted with water (40 mL) and extracted with ethyl acetate (20 mL×2). The combined organic layer was washed with water (20×2 mL) and brine solution (20 mL). Organic layer was dried over anhydrous sodium sulphate and concentrated under reduced pressure to obtain crude compound, which was purified by normal phase combi-flash to obtain desired product.

Step—5: Synthesis of 8-((1R,2R)-2-hydroxy-2-methylcyclopentyl)-2-(methylsulfonyl)-7-oxo-7,8-dihydropyrido[2,3-d]pyrimidine-6-carbonitrile: To a stirred solution of obtain 8-((1R,2R)-2-hydroxy-2-methylcyclopentyl)-2-(methylthio)-7-oxo-7,8-dihydropyrido[2,3-d]pyrimidine-6-carbonitrile (0.102 g, 0.32 mmol, 1 equiv) in DCM (5 mL) was added m-CPBA (77%) (0.066 g, 0.38 mmol, 1.2 equiv) at room temperature. Reaction mass was stirred at room temperature for 2 h. Progress of the reaction was monitored by TLC and LCMS. After completion of the reaction, the reaction mixture was diluted with saturated sodium bicarbonate solution (20 mL) and was extracted with DCM (20 mL×2). The combined organic layer was washed with water (20 mL) and sodium bicarbonate solution (20 mL). Organic layer was dried over anhydrous sodium sulphate and concentrated under reduced pressure to obtain crude 0.1 g (90% yield), which was used for the next step without any purification.

Step—6: Synthesis of 8-((1R,2R)-2-hydroxy-2-methylcyclopentyl)-2-(1-(methylsulfonyl)piperidin-4-ylamino)-7-oxo-7,8-dihydropyrido[2,3-d]pyrimidine-6-carbonitrile: To a stirred solution of 8-((1R,2R)-2-hydroxy-2-methylcyclopentyl)-2-(methylsulfonyl)-7-oxo-7,8-dihydropyrido[2,3-d]pyrimidine-6-carbonitrile (100 mg, 0.22 mmol, 1 equiv) in toluene (3 mL), was added 1-(methylsulfonyl)piperidin-4-amine (0.048 g, 0.27 mmol, 1 equiv) at RT. The resultant reaction mixture was allowed to stir at 100° C. for 2H. Progress of the reaction was monitored by LCMS. After completion of the reaction, solvent was decanted off to obtain crude, which was purified by reverse phase HPLC. LCMS: 446 [M+H]⁺; ¹HNMR: (400 MHz, DMSO-d₆) δ 1.08 (s, 3H)_{1.40}-1.62 (m, 6H) 2.14 (d, J=11.4 Hz, 8H) 2.73-2.98 (s, 3H) 3.58 (br s, 1H) 3.94 (br s, 1H) 4.45 (br s, 1H) 5.82 (br s, 1H) 8.51 (s, 1H) 8.75 (s, 1H).

### Example—S148: Synthesis of 8-cyclopentyl-7-oxo-2-(1-(4-pentylphenylsulfonyl)piperidin-4-ylamino)-7,8-dihydropyrido[2,3-d]pyrimidine-6-carbonitrile (Compound No. 381)

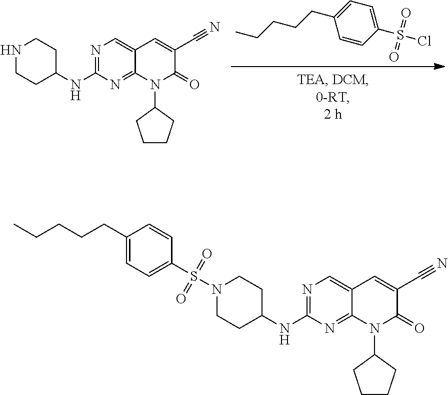

To a stirred solution of 8-cyclopentyl-7-oxo-2-(piperidin-4-ylamino)-7,8-dihydropyrido[2,3-d]pyrimidine-6-carbonitrile hydrochloride (100 mg, 0.26 mmol, 1 equiv) in DCM (5 mL), was added ET₃N (0.1 mL, 0.52 mmol, 2 equiv) and 4-pentylbenzene-1-sulfonyl chloride (65 mg, 0.26 mmol, 1 equiv) at 0° C. Raised the temperature to RT and the reaction mixture was allowed to stir for 2 h. Progress of the reaction was monitored by LCMS. After completion of the reaction, the reaction mixture was diluted with water (15 mL) and extracted with DCM (15 mL). Organic layer was washed with water (10 mL), brine solution (10 mL). Organic layer was dried over anhydrous sodium sulphate and concentrated under reduced pressure to obtain crude, which was triturated from IPA to obtain desired product. LCMS: 549.4 [M+H]⁺; ¹H NMR (400 MHz, DMSO-d₆, VT@ 90° C.) δ 8.65 (s, 1H), 8.40 (s, 1H), 8.20 (d, J=84.1 Hz, 1H), 7.67 (d, J=7.8 Hz, 2H), 7.47 (d, J=7.8 Hz, 2H), 5.68 (s, 1H), 4.02-3.69 (m, 1H), 3.66-3.57 (m, 2H), 2.70 (t, J=7.8 Hz, 2H), 2.23 (s, 3H), 1.99-1.90 (m, 3H), 1.78-1.57 (m, 8H), 1.55 (s, 2H), 1.33 (h, J=8.1, 6.8 Hz, 4H), 0.89 (t, J=6.7 Hz, 3H).

### Example—S149: Synthesis of 8-cyclopentyl-2-(1-(4-hexylphenylsulfonyl)piperidin-4-ylamino)-7-oxo-7,8-dihydropyrido[2,3-d] pyrimidine-6-carbonitrile (Compound No. 382)

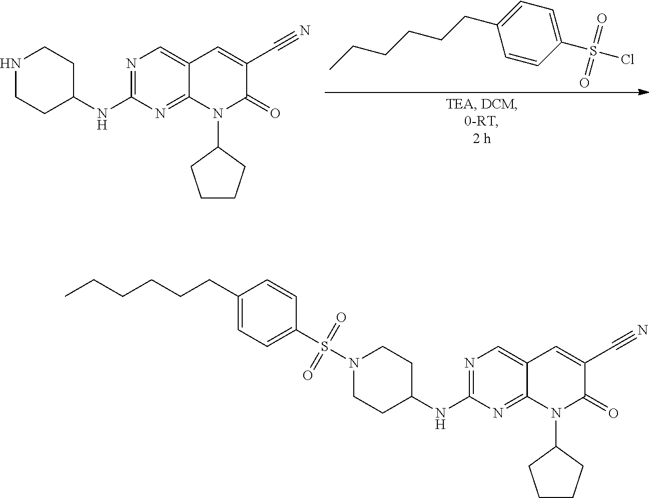 [SMILES: N#Cc1cc2cnc(NC3CCNCC3)nc2n(C2CCCC2)c1=O ; CCCCCCc1ccc(S(=O)(=O)Cl)cc1 ; CCCCCCc1ccc(S(=O)(=O)N2CCC(Nc3ncc4cc(C#N)c(=O)n(C5CCCC5)c4n3)CC2)cc1]

To a stirred solution of 8-cyclopentyl-7-oxo-2-(piperidin-4-ylamino)-7,8-dihydropyrido[2,3-d]pyrimidine-6-carbonitrile hydrochloride (100 mg, 0.26 mmol, 1 equiv) in DCM (5 mL), was added ET₃N (0.1 mL, 0.52 mmol, 2 equiv) and 4-hexylbenzene-1-sulfonyl chloride (70 mg, 0.26 mmol, 1 equiv) at 0° C. Raised the temperature to RT and the reaction mixture was allowed to stir for 2 h. Progress of the reaction was monitored by LCMS. After completion of the reaction, the reaction mixture was diluted with water (15 mL) and extracted with DCM (15 mL). Organic layer was washed with water (10 mL), brine solution (10 mL). Organic layer was dried over anhydrous sodium sulphate and concentrated under reduced pressure to obtain crude, which was triturated from IPA to obtain desired product. LCMS: 563.4 [M+H]⁺; ¹H NMR (400 MHz, DMSO-d₆, VT@ 90° C.) δ 8.65 (s, 1H), 8.40 (s, 1H), 8.34-7.98 (m, 1H), 7.67 (d, J=7.9 Hz, 2H), 7.46 (d, J=7.9 Hz, 2H), 5.76-5.64 (m, 1H), 3.99-3.74 (m, 1H), 3.63 (dd, J=10.6, 5.5 Hz, 2H), 2.70 (t, J=7.7 Hz, 2H), 2.27-2.17 (m, 2H), 1.99-1.90 (m, 3H), 1.87-1.79 (m, 1H), 1.81-1.53 (m, 9H), 1.32 (d, J=9.3 Hz, 7H), 0.91-0.83 (m, 3H).

### Example—S150: Synthesis of 8-cyclopentyl-2-(1-(morpholinosulfonyl)piperidin-4-ylamino)-7-oxo-7,8-dihydropyrido[2,3-d]pyrimidine-6-carbonitrile (Compound No. 383)

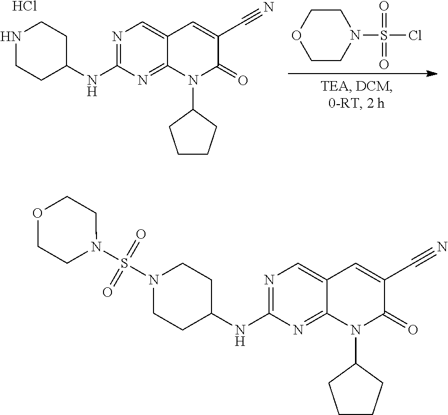

Synthesis of 8-cyclopentyl-2-(1-(morpholinosulfonyl)piperidin-4-ylamino)-7-oxo-7,8-dihydropyrido[2,3-d]pyrimidine-6-carbonitrile: To a stirred solution of 8-cyclopentyl-7-oxo-2-(piperidin-4-ylamino)-7,8-dihydropyrido[2,3-d]pyrimidine-6-carbonitrile hydrochloride (100 mg, 0.26 mmol, 1 equiv) in DCM (5 mL), was added Et₃N (0.1 mL, 0.52 mmol, 2 equiv) and morpholine-4-sulfonyl chloride (49.7 mg, 0.26 mmol, 1 equiv) at 0° C. Raised the temperature to RT and the reaction mixture was allowed to stir at RT for 2 h. Progress of the reaction was monitored by LCMS. After completion of the reaction, the reaction mixture was diluted with water (5 mL) and extracted with DCM (8 mL). Organic layer was washed with water (5 mL), brine solution (5 mL). Organic layer was dried over anhydrous sodium sulphate and concentrated under reduced pressure to obtain crude, which was triturated from IPA to obtain desired product. LCMS: 488.5 [M+H]⁺, 1H NMR (400 MHz, DMSO-d₆) δ ppm 8.73 (s, 1H) 8.48 (s, 1H) 8.34 (d, J=7.8 Hz, 1H) 5.71-5.90 (m, 1H) 4.12 (d, J=7.8 Hz, 1H) 3.59-3.66 (m, 7H) 2.99-3.15 (m, 5H) 2.33 (br s, 1H) 2.27 (br s, 1H) 1.85-2.00 (m, 4H) 1.78 (br s, 2H) 1.53-1.69 (m, 3H)

### Example—S151: Synthesis of 8-(2,2-dimethylcyclopentyl)-2-(1-(methylsulfonyl)piperidin-4-ylamino)-7-oxo-7,8-dihydropyrido[2,3-d]pyrimidine-6-carbonitrile (Compound No. 388)

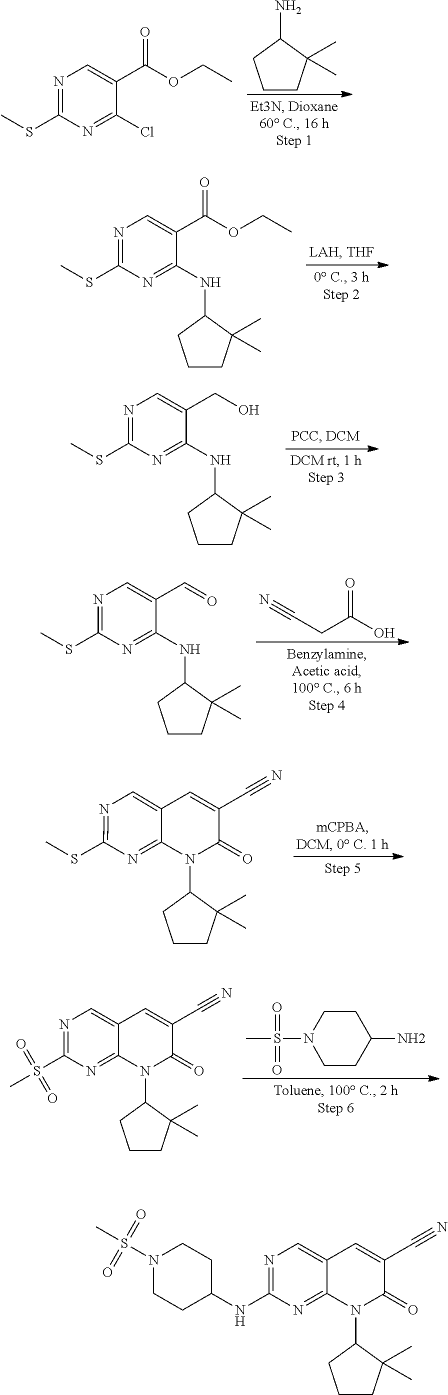

Step—1: Synthesis of ethyl 4-(2,2-dimethylcyclopentylamino)-2-(methylthio)pyrimidine-5-carboxylate: To a stirred solution of ethyl 4-chloro-2-(methylthio)pyrimidine-5-carboxylate (2.7 g, 11.63 mmol, 1 equiv) in dioxane (30 mL), was added TEA (0.9 mL, 17.45 mmol, 3.0 equiv) and 2,2-dimethylcyclopentanamine (2.25 g, 15.12 mmol, 1.2 equiv). The resultant reaction mixture was allowed to stir for 2 h at 50° C. Progress of the reaction was monitored by TLC and LCMS. After completion of the reaction, the reaction mixture was diluted with water (100 mL) and extracted with ethyl acetate (150 mL×2). Organic layer was washed with water (150 mL), brine solution (150 mL). Organic layer was dried over anhydrous sodium sulphate and concentrated under reduced pressure to obtain crude, which was used for the next step without any further purification.

Step—2: Synthesis of (4-(2,2-dimethylcyclopentylamino)-2-(methylthio)pyrimidin-5-yl)methanol: To a stirred solution of ethyl 4-(2,2-dimethylcyclopentylamino)-2-(methylthio)pyrimidine-5-carboxylate (2.6 g, 8.4 mmol, 1 equiv) in THF (30 mL), was added LAH (0.64 g, 16.8 mmol, 2 equiv) at 0° C. Raise the temp. to RT and the reaction mixture was allowed to stir for 5 h. Progress of the reaction was monitored by TLC and LCMS. After completion of the reaction, the reaction mixture was quenched with saturated solution of sodium hydroxide (20 mL) at 0° C., the reaction mixture was then passes through celite bed, filtrate obtain was concentrated under reduced pressure to obtain crude, which was used for the next step without any further purification.

Step—3: Synthesis of 4-(2,2-dimethylcyclopentylamino)-2-(methylthio)pyrimidine-5-carbaldehyde: To a stirred solution of (4-(2,2-dimethylcyclopentylamino)-2-(methylthio)pyrimidin-5-yl)methanol (2.0 g, 7.46 mmol, 1 equiv) in DCM (30 mL), was added PCC (1.6 g, 7.46 mmol, 1 equiv) at RT. The reaction mixture was allowed to stir at RT for 1 h. Progress of the reaction was monitored by TLC and LCMS. After completion of the reaction, the reaction mixture was passes through celite bed; filtrate was concentrated under reduced pressure to obtain crude, which was purified by normal phase combi flash to obtain desired product.

Step—4: Synthesis of 8-(2,2-dimethylcyclopentyl)-2-(methylthio)-7-oxo-7,8-dihydropyrido[2,3-d]pyrimidine-6-carbonitrile: To a stirred solution of 4-(2,2-dimethylcyclopentylamino)-2-(methylthio)pyrimidine-5-carbaldehyde (0.350 g, 1.31 mmol, 1.0 equiv.) in Acetic acid (3 mL), was added cyanoacetic acid (0.334 g, 3.93 mmol, 3 equiv) and benzylamine (0.014 g, 0.131 mmol, 0.1 equiv). The reaction mixture was allowed to stir at 100° C. for overnight. Progress of the reaction was monitored by TLC and LCMS. After completion of the reaction, the reaction mixture was diluted with water (40 mL) and extracted with ethyl acetate (20 mL×2). The combined organic layer was washed with water (20 mL×2) and brine solution (20 mL). Organic layer was dried over anhydrous sodium sulphate and concentrated under reduced pressure to obtain crude compound, which was purified by normal phase combi-flash to obtain 0.130 g of desired product.

Step—5: Synthesis of 8-(2,2-dimethylcyclopentyl)-2-(methylsulfonyl)-7-oxo-7,8-dihydropyrido[2,3-d]pyrimidine-6-carbonitrile: To a stirred solution of obtain 8-(2,2-dimethylcyclopentyl)-2-(methylthio)-7-oxo-7,8-dihydropyrido[2,3-d]pyrimidine-6-carbonitrile (0.130 g, 0.41 mmol, 1 equiv) in DCM (5 mL) was added m-CPBA (77%) (0.106 g, 0.61 mmol, 1.5 equiv) portion wise at room temperature. Reaction mass was stirred at room temperature for 2 h. Progress of the reaction was monitored by TLC and LCMS. After completion of the reaction, the reaction mixture was diluted with saturated sodium bicarbonate solution (20 mL) and was extracted with DCM (20 mL×2). The combined organic layer was washed with water (20 mL) and sodium bicarbonate solution (20 mL). Organic layer was dried over anhydrous sodium sulphate and concentrated under reduced pressure to obtain crude 0.1 g, which was used for the next step without any purification.

Step—6: Synthesis of 8-(2,2-dimethylcyclopentyl)-2-(1-(methylsulfonyl)piperidin-4-ylamino)-7-oxo-7,8-dihydropyrido[2,3-d]pyrimidine-6-carbonitrile: To a stirred solution of 8-(2,2-dimethylcyclopentyl)-2-(methylsulfonyl)-7-oxo-7,8-dihydropyrido[2,3-d]pyrimidine-6-carbonitrile (0.120 mg, 0.34 mmol, 1 equiv) in toluene (3 mL), was added 1-(methylsulfonyl)piperidin-4-amine (0.074 g, 0.41 mmol, 1.2 equiv) at RT. The resultant reaction mixture was allowed to stir at 100° C. for 2H. Progress of the reaction was monitored by LCMS. After completion of the reaction, solvent was decanted off to obtain crude, which was purified by reverse phase HPLC to obtain desired product. LCMS: 445.5 [M+H]⁺; ¹HNMR: (400 MHz, DMSO-d₆: δ 8.67-8.81 (m, 1H) 8.61 (d, J=7.4 Hz, 1H) 5.64 (br s, 1H) 3.94 (br s, 1H) 3.59 (br s, 2H) 2.85-3.00 (m, 3H) 2.81 (br s, 1H) 2.33 (br s, 6H) 1.97 (br s, 5H) 1.75 (br s, 1H) 1.66 (s, 3H) 1.46 (br s, 1H) 1.14 (s, 2H).

### Example—S152: Synthesis of 8-cyclopentyl-5-methyl-2-(1-(methylsulfonyl)piperidin-4-ylamino)-7-oxo-7,8-dihydropyrido[2,3-d]pyrimidine-6-carbonitrile (Compound No. 389)

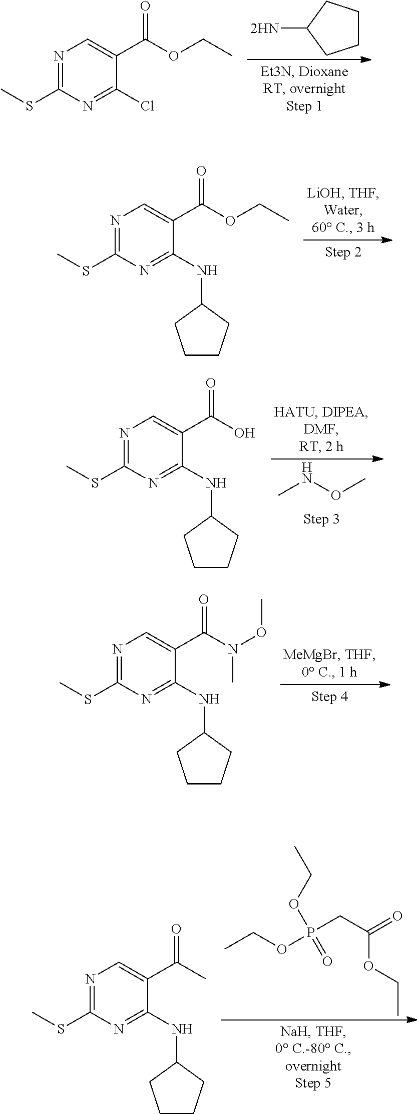

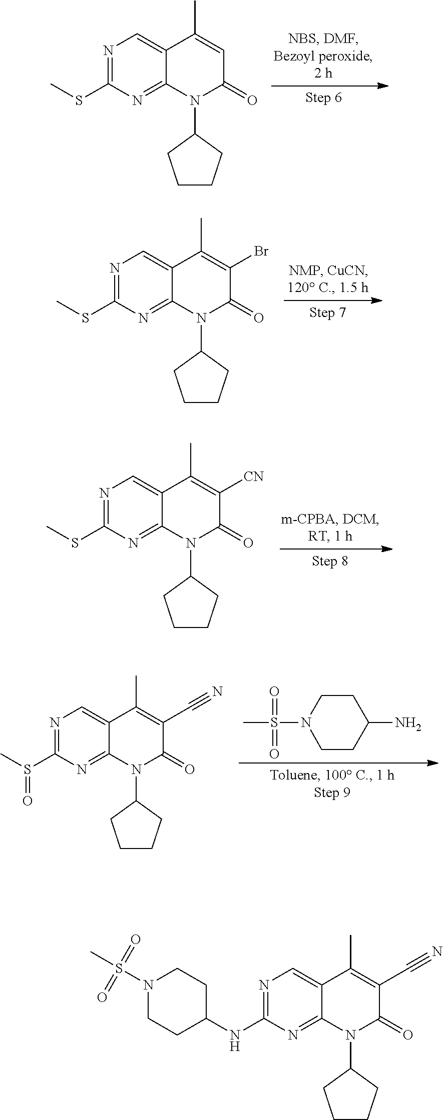

Step—1: Synthesis of ethyl 4-(cyclopentylamino)-2-(methylthio)pyrimidine-5-carboxylate: To a stirred solution of ethyl 4-chloro-2-(methylthio)pyrimidine-5-carboxylate (5000 mg, 21.55 mmol, 1 equiv) in Dioxane (50 mL), was added ET₃N (4.5 mL, 32.3 mmol, 1.5 equiv) and cyclopentylmethanamine (2193 mg, 25.8 mmol, 1.2 equiv) at RT. The resultant reaction mixture was allowed to stir for overnight. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain crude, which was used for the next step without any further purification.

Step—2: Synthesis of 4-(cyclopentylamino)-2-(methylthio)pyrimidine-5-carboxylic acid: To a stirred solution of ethyl 4-(cyclopentylamino)-2-(methylthio)pyrimidine-5-carboxylate (5000 mg, 21.5 mmol, 1 equiv) in THF: WATER (1:1=50 mL), was added LiOH (5170 mg, 0215 mmol, 10 equiv) at RT. Then the reaction mixture was allowed to stir for 3 h at 60° C. Progress of the reaction was monitored by LCMS. After completion of the reaction, the reaction mixture was diluted with water (100 mL), solid observed was filtered and dried under vacuum to obtain crude, which was used for the next step without any further purification.

Step—3: Synthesis of 4-(cyclopentylamino)-N-methoxy-N-methyl-2-(methylthio)pyrimidine-5-carboxamide: To a stirred solution of 4-(cyclopentylamino)-2-(methylthio)pyrimidine-5-carboxylic acid (4000 mg, 15.81 mmol, 1 equiv) in DMF (20 mL), was added HATU (10814 mg, 28.45 mmol, 1.8 equiv), DIPEA (11 mL, 63.24 mmol, 4 equiv) and Methoxymethyl amine (1840 mg, 15.8 mmol, 1.2 equiv) at RT. The resultant reaction mixture was allowed to stir for 2 h. Progress of the reaction was monitored by TLC and LCMS. After completion of the reaction, the reaction mixture was diluted with water (100 mL) and extracted with ethyl acetate (150 mL×2). Organic layer was washed with water (100 mL), brine solution (100 mL). Organic layer was dried over anhydrous sodium sulphate and concentrated under reduced pressure to obtain crude, which was purified by normal phase combi flash to obtain desired product.

Step—4: Synthesis of 1-(4-(cyclopentylamino)-2-(methylthio)pyrimidin-5-yl)ethanone: To a stirred solution of 4-(cyclopentylamino)-N-methoxy-N-methyl-2-(methylthio)pyrimidine-5-carboxamide (1500 mg, 5.06 mmol, 1 equiv) in THF (20 mL), was added MeMgBr (3M in diethyl ether) (5 mL, 20.27 mmol, 4 equiv) at 0° C. The resultant reaction mixture was allowed to stir for 1 h. Progress of the reaction was monitored by TLC and LCMS. After completion of the reaction, the reaction mixture was diluted with water (50 mL) and extracted with ethyl acetate (150 mL). Organic layer was washed with water (100 mL), brine solution (100 mL). Organic layer was dried over anhydrous sodium sulphate and concentrated under reduced pressure to obtain crude, which was purified by normal phase combi flash to obtain desired product.

Step—5: Synthesis of 8-cyclopentyl-5-methyl-2-(methylthio)pyrido[2,3-d]pyrimidin-7(8H)-one: To a stirred solution of NaH (60% in mineral oil) (1106 mg, 29 mmol, 29 equiv) in THF (30 mL), was added triethyl phosphonoacetate (5.7 mL, 29 mmol, 4 equiv) at 0° C. The cooling bath was removed and a solution of 1-(4-(cyclopentylamino)-2-(methylthio)pyrimidin-5-yl)ethanone (2800 mg, 11.15 mmol, 1 equiv) in THF (5 mL) was added. The resultant reaction mixture was allowed to stir for overnight at 80° C. under reflux condition. Progress of the reaction was monitored by TLC and LCMS. After completion of the reaction, the reaction mixture was diluted with ice water (100 mL), solid observed was filtered and dried under vacuum to obtain crude, which was used for the next step without any further purification.

Step—6: Synthesis of 6-bromo-8-cyclopentyl-5-methyl-2-(methylthio)pyrido[2,3-d]pyrimidin-7(8H)-one: To a stirred solution of 8-cyclopentyl-5-methyl-2-(methylthio)pyrido[2,3-d]pyrimidin-7(8H)-one (1500 mg, 5.45 mmol, 1 equiv) in DMF (7 mL), was added N-bromosuccinimide (1456 mg, 8.18 mmol, 1.5 equiv) and benzoyl peroxide (131 mg, 0.5 mmol, 0.1 equiv) at RT. The resultant reaction mixture was allowed to stir for 2 h. Progress of the reaction was monitored by TLC and LCMS. After completion of the reaction, the reaction mixture was diluted with ice water (50 mL) and extracted with ethyl acetate (150 mL). Organic layer was washed with water (100 mL), brine solution (100 mL). Organic layer was dried over anhydrous sodium sulphate and concentrated under reduced pressure to obtain crude, which was purified by normal phase combi flash to obtain desired product.

Step—7: Synthesis of 8-cyclopentyl-5-methyl-2-(methylthio)-7-oxo-7,8-dihydropyrido[2,3-d]pyrimidine-6-carbonitrile: To a stirred solution of 6-bromo-8-cyclopentyl-5-methyl-2-(methylthio)pyrido[2,3-d]pyrimidin-7(8H)-one (200 mg, 0.56 mmol, 1 equiv) in NMP (2 mL), was added CuCN (152 mg, 1.70 mmol, 3 equiv). The resultant reaction mixture was allowed to stir for 1.5 h at 120° C. in microwave. Progress of the reaction was monitored by TLC and LCMS. After completion of the reaction, the reaction mixture was diluted with ice water (20 mL) and extracted with ethyl acetate (100 mL). Organic layer was washed with water (50 mL×5), brine solution (50 mL). Organic layer was dried over anhydrous sodium sulphate and concentrated under reduced pressure to obtain crude, which was purified by normal phase combi flash to obtain desired product.

Step—8: Synthesis of 8-cyclopentyl-5-methyl-2-(methylsulfinyl)-7-oxo-7,8-dihydropyrido[2,3-d]pyrimidine-6-carbonitrile: To a stirred solution of 8-cyclopentyl-5-methyl-2-(methylthio)-7-oxo-7,8-dihydropyrido[2,3-d]pyrimidine-6-carbonitrile (100 mg, 0.33 mmol, 1 equiv) in DCM (3 mL), was added m-CPBA (77 mg, 0.43 mmol, 1.3 equiv) at RT. Then the reaction mixture was allowed to stir for 1 h. Progress of the reaction was monitored by LCMS. After completion of the reaction, the reaction mixture was diluted with DCM (30 mL) and washed with water (3 mL), brine solution (30 mL). Organic layer was dried over anhydrous sodium sulphate and concentrated under reduced pressure to obtain crude, which was used for the next step without any further purification.

Step—9: Synthesis of 8-cyclopentyl-5-methyl-2-(1-(methylsulfonyl)piperidin-4-ylamino)-7-oxo-7,8-dihydropyrido[2,3-d]pyrimidine-6-carbonitrile: To a stirred solution of 8-cyclopentyl-5-methyl-2-(methylsulfinyl)-7-oxo-7,8-dihydropyrido[2,3-d]pyrimidine-6-carbonitrile (80 mg, 0.25 mmol, 1 equiv) in toluene (3 mL), was added 1-(methylsulfonyl)piperidin-4-amine (54 mg, 0.3 mmol, 1.2 equiv). The resultant reaction mixture was allowed to stir at 100° C. for 1 h. Progress of the reaction was monitored by LCMS. After completion of the reaction, solvent was removed under reduced pressure to obtain crude, which was purified by reverse phase HPLC to afford desired product. LCMS: 431.5 [M+H]⁺, ¹H NMR (400 MHz, DMSO-d₆, VT@ 90° C.) δ 8.88 (s, 1H), 8.36-7.87 (m, 1H), 5.82 (s, 1H), 4.00 (d, J=18.1 Hz, 1H), 3.67-3.57 (m, 2H), 2.90 (d, J=11.2 Hz, 5H), 2.61 (s, 3H), 2.25 (s, 2H), 2.04-1.94 (m, 4H), 1.85-1.73 (m, 2H), 1.66 (t, J=11.6 Hz, 4H).

### Example—S153: Synthesis of 8-[(1R,3R,5S)-bicyclo[3.1.0]hexan-3-yl]-2-(methylsulfanyl)pyrido[2,3-d]pyrimidin-7-one

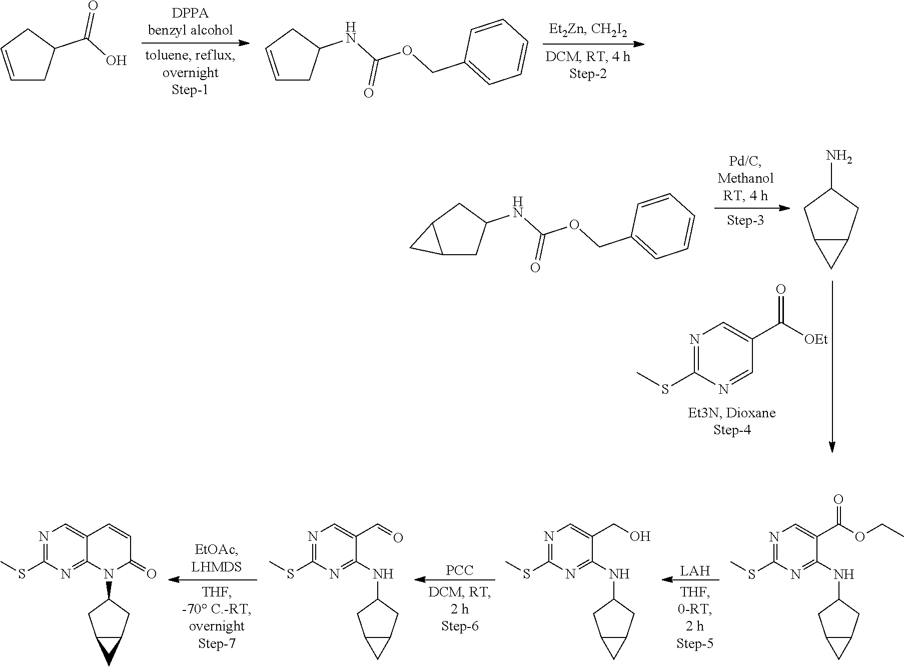

Step 1: Synthesis of benzyl cyclopent-3-en-1-ylcarbamate: To a solution of cyclopent-3-ene-1-carboxylic acid (50 g, 446 mmol) and DPPA (135 g, 490 mmol) in toluene (800 mL) was added Et₃N (74 mL, 535 mmol) at RT. The mixture was then stirred at reflux for 2 h. benzyl alcohol (70 mL, 669 mmol) was then added at RT, the resulting mixture was stirred at 100° C. overnight and cooled to room temperature. The reaction mixture was quenched with saturated aqueous NaHCO₃. The resulting mixture was extracted with Ethyl acetate. Combined organic layers were washed with brine, dried over anhydrous Na2SO₄, and concentrated under reduced pressure. The residue was purified by silica gel chromatography to afford the title compound (55 g, 57%). LCMS: 218.1 [M+H]⁺

Step 2: Synthesis of Benzyl bicyclo[3.1.0]hexan-3-ylcarbamate: To a solution of benzyl cyclopent-3-en-1-ylcarbamate (55 g, 253.1 mmol) in DCM (430 mL) was added ZnEt₂ (1 M, 380 mL, 380 mmol) at 0° C. followed by the addition of CH₂I₂ (30.1 mL, 380 mmol) at 0° C. The reaction mixture was allowed to warm to RT and stirred for 4 h. The resulting mixture was washed with brine, dried over Na2SO₄, filtered and the solvent was concentrated. The residue was purified by silica gel chromatography to afford the title compound (42 g, 72%). LCMS: 232.2 [M+H]⁺

Step 3: Synthesis of Bicyclo[3.1.0]hexan-3-amine: To a solution of benzyl bicyclo [3.1.0]hexan-3-ylcarbamate (5 g, 21.6 mmol) in MeOH (50 mL) at RT under an atmosphere of nitrogen was added Pd/C (2 g) in one portion. The resulting mixture was then stirred under a hydrogen (0.4 MPa) at 40° C. The reaction mixture was filtered, and the filtrate was concentrated under reduced pressure to give the desired product (2.0 g, 96%) which was used directly in the next step without any further purification. LCMS: 98 [M+H]⁺

Step 4: Synthesis of Ethyl-4-(bicyclo[3.1.0]hexan-3-ylamino)-2-(methylthio)pyrimidine-5-carboxylate: To a stirred solution of ethyl 4-chloro-2-(methylthio)pyrimidine-5-carboxylate (6.0 g, 25.7 mmol) in Dioxane (60 mL), were added Et₃N (10.8 mL, 30.9 mmol) and bicyclo[3.1.0]hexan-3-amine (2.00 g, 20.6 mmol) at RT. The resultant reaction mixture was then allowed to stir for overnight at RT. Progress of the reaction was monitored by TLC and LCMS. After completion of the reaction, the reaction mixture was diluted with water (100 mL) and extracted with ethyl acetate (150 mL×2). The combined organic layer was washed with water (100 mL), brine solution (100 mL), then dried over Na2SO₄ and concentrated under reduced pressure. The residue was purified by silica gel chromatography to afford the title compound (2.3 g, 30%). LCMS: 294.1 [M+H]⁺

Step 5: Synthesis of (4-(bicyclo[3.1.0]hexan-3-ylamino)-2-(methylthio)pyrimidin-5-yl)methanol: To a stirred solution of Ethyl 4-(bicyclo[3.1.0]hexan-3-ylamino)-2-(methylthio)pyrimidine-5-carboxylate (2.3 g, 7.84 mmol) in THF (30 mL), was added LAH (596 mg, 15.69 mmol) at 0° C. The reaction mixture was allowed to stir at RT for 2 h. Progress of the reaction was monitored by TLC and LCMS. After completion of the reaction, the reaction mixture was quenched with water (12 mL) followed by the addition of 10% solution of sodium hydroxide (6 mL) at 0° C. and stirred for 10 min at RT. The resulting mixture was filtered through celite and dried over anhydrous Na2SO₄ and concentrated under reduced pressure to obtain the desired product (1.95 g, 99%). LCMS: 252.1 [M+H]⁺

Step 6: Synthesis of 4-(bicyclo[3.1.0]hexan-3-ylamino)-2-(methylthio)pyrimidine-5-carbaldehyde: To a stirred solution of (4-(bicyclo[3.1.0]hexan-3-ylamino)-2-(methylthio)pyrimidin-5-yl)methanol (1.95 g, 7.8 mmol) in DCM (20 mL), was added PCC (1.85 g, 8.6 mmol) at RT. The reaction mixture was then allowed to stir at RT for overnight. Progress of the reaction was monitored by TLC and LCMS. After completion of the reaction, the reaction mixture was passed through celite bed, filtrate obtained was diluted with DCM (150 mL) and washed with water (100 mL), brine solution (100 mL). Organic layer was dried over anhydrous Na₂SO₄ and concentrated under reduced pressure to obtain crude, which was purified by silica gel chromatography to afford the title compound (1.9 g, 98%). LCMS: 250.0 [M+H]⁺

Step 7: Synthesis of 8-[(1R,3R,5S)-bicyclo[3.1.0]hexan-3-yl]-2-(methylsulfanyl)pyrido[2,3-d]pyrimidin-7-one: To a solution of 4-(bicyclo[3.1.0]hexan-3-ylamino)-2-(methylthio)pyrimidine-5-carbaldehyde (1.9 g, 5.44 mmol) in anhydrous THF was added EtOAc (1.44 g, 16.32 mmol) at −70° C. The mixture was stirred at the temperature for 15 min. Then LHDMS (19.0 mL, 19.04 mmol) was added dropwise. The reaction was stirred at −70° C. for 30 min and then at 20° C. for overnight. The solution was cooled in an ice bath, quenched with water, and then extracted with Ethyl Acetate (50 mL×3). The combined organic layers were washed with aq. NH₄Cl (30 mL), and brine (30 mL), dried over Na2SO₄, and concentrated. The residue was purified by silica gel chromatography to afford the title compound (1.0 g, 50%). LCMS: 274.0 [M+H]+. ¹H NMR (400 MHz, DMSO-d₆) δ 8.87 (s, 1H), 7.87 (d, J=9.4 Hz, 1H), 6.54 (d, J=9.4 Hz, 1H), 6.15-6.13 (m, 1H), 2.60 (s, 3H), 2.12-2.10 (m, 4H), 1.43-1.35 (m, 2H), 0.90 (m, 1H), 0.87-0.80 (m, 1H).

### Example—S154: Synthesis of 8-{bicyclo[1.1.1]pentan-1-yl}-2-(methylsulfanyl)pyrido[2,3-d]pyrimidin-7-one

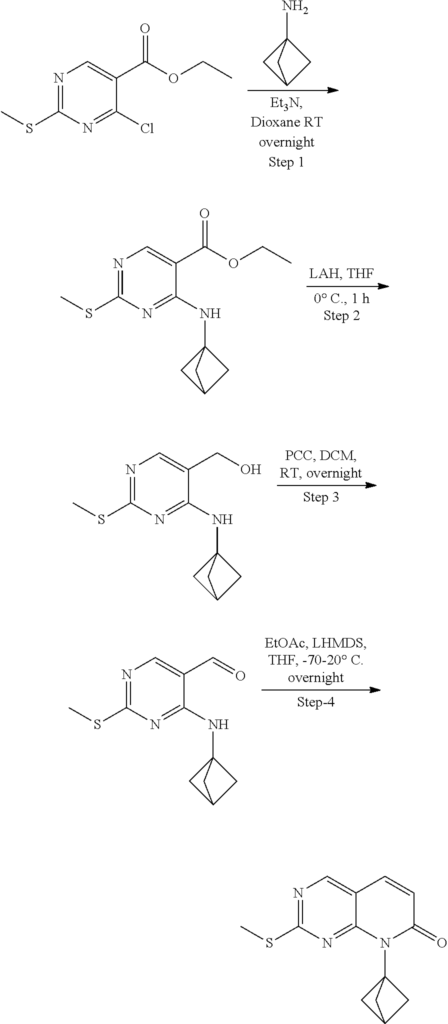

Step—1: Synthesis of ethyl 4-(bicyclo[1.1.l]pentan-1-ylamino)-2-(methylthio)pyrimidine-5-carboxylate: To a stirred solution of ethyl 4-chloro-2-(methylthio)pyrimidine-5-carboxylate (40 g, 0.17 mol) in Dioxane (400 mL), were added Et₃N (72 mL, 0.515 mol) and bicyclo[1.1.1]pentan-1-amine (24.72 g, 0.206 mol) at RT. The resultant reaction mixture was allowed to stir for overnight. Progress of the reaction was monitored by TLC and LCMS. After completion of the reaction, the reaction mixture was diluted with water (500 mL) and extracted with ethyl acetate (600 mL). Organic layer was washed with water (100 mL), brine solution (100 mL). Organic layer was dried over anhydrous Na2SO₄ and concentrated under reduced pressure to obtain crude. The residue was purified by silica gel chromatography to afford the title compound (42 g, 88%). LCMS: 280.1 [M+H]⁺

Step—2: Synthesis of (4-(bicyclo[1.1.1]pentan-1-ylamino)-2-(methylthio)pyrimidin-5-yl)methanol: To a stirred solution of ethyl 4-(bicyclo[1.1.1]pentan-1-ylamino)-2-(methylthio)pyrimidine-5-carboxylate (42 g, 150.4 mmol) in THF (500 mL), was added LAH (11.42 g, 300.72 mmol) at 0° C. Raise the temperature To RT and the reaction mixture was allowed to stir for 1 h. Progress of the reaction was monitored by TLC and LCMS. After completion of the reaction, the reaction mixture was quenched with saturated solution of sodium hydroxide (5 mL) at 0° C., the reaction mixture was then passed through celite bed, filtrate obtained was concentrated under reduced pressure to obtain the desired product (26 g, 73%), which was used for the next step without any further purification. LCMS: 238.1 [M+H]⁺

Step—3: Synthesis of 4-(bicyclo[1.1.1]pentan-1-ylamino)-2-(methylthio)pyrimidine-5-carbaldehyde: To a stirred solution of (4-(bicyclo [1.1.1] pentan-1-ylamino)-2-(methylthio) pyrimidin-5-yl) methanol (26 g, 109.53 mmol) in DCM (300 mL), was added PCC (23.7 g, 109.53 mmol) at RT. The reaction mixture was allowed to stir at RT for overnight. Progress of the reaction was monitored by TLC and LCMS. After completion of the reaction, the reaction mixture was passed through celite bed; filtrate was concentrated under reduced pressure to obtain the desired product (22 g, 88%), which was used for the next step without any further purification. LCMS: 236.1 [M+H]⁺

Step 4: Synthesis of 8-{bicyclo[1.1.1]pentan-1-yl}-2-(methylsulfanyl)pyrido[2,3-d]pyrimidin-7-one: To a solution of 4-(bicyclo[1.1.1]pentan-1-ylamino)-2-(methylthio)pyrimidine-5-carbaldehyde (22 g, 0.09 mol) in anhydrous THF was added EtOAc (24.86 g, 0.28 mol) at −70° C. The mixture was stirred at the temperature for 15 min. Then LHDMS (327.8 mL, 0.33 mol) was added dropwise. The reaction was stirred at −70° C. for 30 min and then at 20° C. for 16 h. the solution was cooled in an ice bath, quenched with water, and then extracted with Ethyl Acetate (500 mL×3). The combined organic layers were washed with aq. NH₄Cl (300 mL) and brine (300 mL), dried over Na2SO₄, and concentrated. The residue was concentrated under reduced pressure to obtain the desired product (15 g, 62.5%). LCMS: 260.0 [M+H]+. ¹H NMR (400 MHz, DMSO-d₆) δ 8.82 (s, 1H), 7.84 (d, J=9.4 Hz, 1H), 6.50 (d, J=9.4 Hz, 1H), 2.63-2.61 (m, 1H), 2.61-2.59 (m, 6H), 2.58 (s, 3H).

### Example—S155: Synthesis of 8-[(S,2S,4R)-bicyclo[2.2.1]heptan-2-yl]-2-(methylsulfanyl)pyrido[2,3-d]pyrimidin-7-one

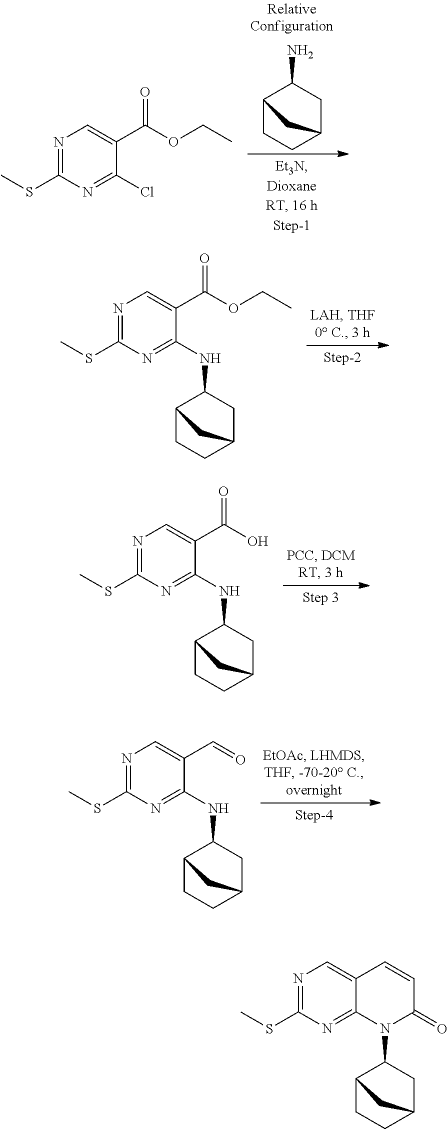

Step—1: Synthesis of Ethyl-4-((1S,2S,4R)-bicyclo[2.2.1]heptan-2-ylamino)-2-(methylthio)pyrimidine-5-carboxylate: To a stirred solution of ethyl 4-chloro-2-(methylthio)pyrimidine-5-carboxylate (40 g, 171.84 mmol) in 1,4-Dioxane (400 mL), were added Et₃N (72 mL, 515.7 mmol) and (1S,2S,4R)-bicyclo[2.2.1]heptan-2-amine (relative configuration) (28 g, 189.1 mmol) at RT. The resultant reaction mixture was then allowed to stir for overnight at RT. Progress of the reaction was monitored by TLC and LCMS. After completion of the reaction, the reaction mixture was diluted with water (300 mL) and extracted with ethyl acetate (450 mL×2). The combined organic layer was washed with water (100 mL), brine solution (200 mL), then dried over anhydrous Na2SO₄ and concentrated under reduced pressure to obtain the desired product (46 g, 87%). LCMS: 308 [M+H]⁺

Step—2: Synthesis of (4-((1S,2S,4R)-bicyclo[2.2.1]heptan-2-ylamino)-2-(methylthio)pyrimidin-5-yl)methanol: To a stirred solution of Ethyl-4-((1S,2S,4R)-bicyclo[2.2.1]heptan-2-ylamino)-2-(methylthio)pyrimidine-5-carboxylate (46 g, 149.6 mmol) in THF (500 mL), was added LAH (10.35 g, 299.23 mmol) at 0° C. The reaction mixture was allowed to stir at 0° C. for 3 h. Progress of the reaction was monitored by TLC and LCMS. After completion of the reaction, the reaction mixture was quenched with saturated solution of NaOH (200 mL) at 0° C. The residue was filtered through celite bed and washed with ethyl acetate (100 mL). Filtrate was extracted with ethyl acetate (300 mL×2). The combined organic layer was washed with water (200 mL) and brine solution (200 mL), then dried over anhydrous Na2SO4 and concentrated under reduced pressure to obtain the desired product (30 g, 78%). LCMS: 266 [M+H]⁺

Step—3: Synthesis of 4-((1S,2S,4R)-bicyclo[2.2.1]heptan-2-ylamino)-2-(methylthio)pyrimidine-5-carbaldehyde: To a stirred solution of (4-((1S,2S,4R)-bicyclo[2.2.1]heptan-2-ylamino)-2-(methylthio)pyrimidin-5-yl)methanol (30 g, 113 mmol) in DCM (300 mL), was added PCC (29.2 g, 135.6 mmol) at RT. The reaction mixture was then allowed to stir at RT for overnight. Progress of the reaction was monitored by TLC and LCMS. After completion of the reaction, the reaction mixture was passed through celite bed, filtrate obtain was diluted with DCM (500 mL) and washed with water (200 mL), brine solution (300 mL). Organic layer was dried over anhydrous Na2SO4 and concentrated under reduced pressure to obtain crude, which was purified by silica gel chromatography to afford the title compound (27 g, 94%). LCMS: 264 [M+H]⁺

Step 4: Synthesis of 8-[(1S,2S,4R)-bicyclo[2.2.1]heptan-2-yl]-2-(methylsulfanyl)pyrido[2,3-d]pyrimidin-7-one: To a solution of 4-[(1S,2S,4R)-bicyclo[2.2.1]heptan-2-ylamino]-2-(methylsulfanyl)pyrimidine-5-carbaldehyde (27 g, 0.10 mol) in anhydrous THF was added EtOAc (27 g, 0.31 mol) at −70° C. The mixture was stirred at the temperature for 15 min. Then LHDMS (359.1 mL, 0.36 mol) was added dropwise. The reaction was stirred at −70° C. for 30 min and then at 20° C. for 16 h. The solution was cooled in an ice bath, quenched with water, and then extracted with Ethyl Acetate (500 mL×3). The combined organic layers were washed with aqueous NH₄Cl (300 mL) and brine (300 mL), dried over Na2SO4, and concentrated. The residue was purified by silica gel chromatography to afford the title compound (15 g, 50%). LCMS: 288 [M+H]+. ¹H NMR (400 MHz, DMSO-d₆) δ 8.84 (s, 1H), 7.85 (d, J=9.4 Hz, 1H), 6.53 (d, J=9.4 Hz, 1H), 5.28-5.22 (m, 1H), 2.59 (s, 3H), 2.56 (d, J=9.2 Hz, 1H), 2.43 (s, 1H), 2.38 (d, J=2.2 Hz, 1H), 2.36-2.31 (m, 1H), 1.68-1.66 (m, 1H), 1.61-1.50 (m, 2H), 1.36-1.34 (m, 1H), 1.30-1.22 (m, 1H), 1.18-1.15 (m, 1H).

### Example—S156: Synthesis of 8-((1R,3R,5S)-bicyclo[3.1.0]hexan-3-yl)-6-(difluoromethyl)-2-((1-(methylsulfonyl)piperidin-4-yl)amino)pyrido[2,3-d]pyrimidin-7(8H)-one (Compound 196)

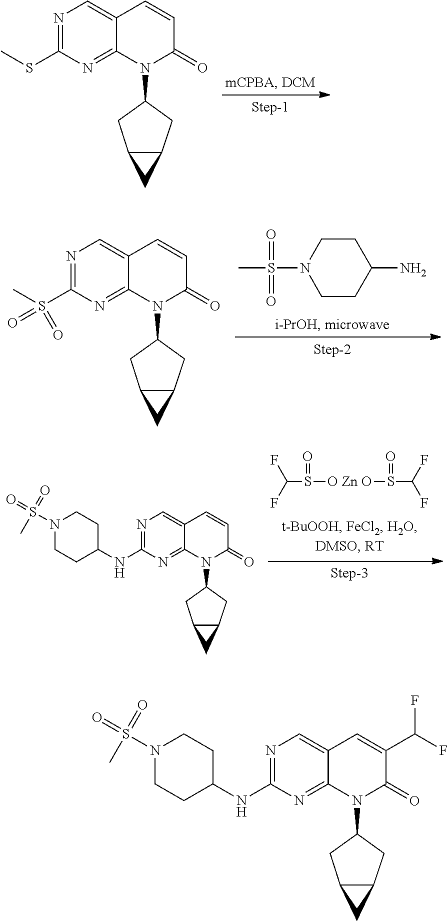

Step—1: Synthesis of 8-((1R,3R,5S)-bicyclo[3.1.0]hexan-3-yl)-2-(methylsulfonyl)pyrido[2,3-d]pyrimidin-7(8H)-one: To a solution of 8-((1R,3R,5S)-bicyclo[3.1.0]hexan-3-yl)-2-(methylsulfonyl)pyrido[2,3-d]pyrimidin-7(8H)-one (970 mg, 3.55 mmol, 1.0 eq) in CH₂Cl₂ (40 mL) was added mCPBA (1.8 g, 8.88 mmol, 2.5 eq) portion wise. The mixture was running at RT for 1 h. The mixture was quenched with saturated aqueous Na2SO3 (40 mL). The organic layer was washed with saturated aqueous Na2CO₃ (40 mL×2) and brine (40 mL). The organic layer was dried over anhydrous Na2SO4. After filtration, the solvent was removed in vacuum and the residue was purified by silica gel chromatography to afford the title compound (770 mg, 71%). LCMS: 306.0 [M+H]⁺

Step—2: Synthesis of 8-((1R,3R,5S)-bicyclo[3.1.0]hexan-3-yl)-2-((1-(methylsulfonyl)piperidin-4-yl)amino)pyrido[2,3-d]pyrimidin-7(8H)-one: To a sealed microwave tube was added a solution of 8-((1R,3R,5S)-bicyclo[3.1.0]hexan-3-yl)-2-(methylsulfonyl)pyrido[2,3-d]pyrimidin-7(8H)-one (380 mg, 1.25 mmol, 1.0 eq), 1-(methylsulfonyl)piperidin-4-amine (333 mg, 1.88 mmol, 1.5 eq) in i-PrOH (12 mL), and the reaction was heated at 130° C. under microwave for 1 h. The solvent was removed in vacuum and the residue was dissolved in CH₂Cl₂ (50 mL). The mixture was washed with H₂O (50 mL) and brine (50 mL). The organic phase was dried over anhydrous Na2SO4. After filtrate, the solvent was removed in vacuum and the residue was purified by silica gel chromatography to afford the title compound (350 mg, 69%). LCMS: 404.2 [M+H]⁺

Step—3: Synthesis of 8-((1R,3R,5S)-bicyclo[3.1.0]hexan-3-yl)-6-(difluoromethyl)-2-((1-(methylsulfonyl)piperidin-4-yl)amino)pyrido[2,3-d]pyrimidin-7(8H)-one: To a solution of Zinc difluoromethanesulfinate (770 mg, 2.61 mmol, 3.0 eq) and FeC₂.4H₂O (88 mg, 0.44 mmol, 0.51 eq) in water (3 mL) was added dropwise to a solution of 8-((1R,3R,5S)-bicyclo[3.1.0]hexan-3-yl)-2-((1-(methylsulfonyl)piperidin-4-yl)amino)pyrido[2,3-d]pyrimidin-7(8H)-one (350 mg, 0.87 mmol, 1.0 eq) and TFA (99 mg, 0.87 mmol, 1.0 eq) in DMSO (14 mL) under N₂ atmosphere at RT. The resulting mixture was treated with TBHP (5-6N, 0.2 mL). Stirring was continued at RT for 19 h. A second portion of TBHP (0.2 mL) was added and stirring continued for 3 h. A third portion of TBHP (0.2 mL) was added and stirring at RT for 45 min. The reaction solution was poured into a mixture of 10% aqueous sodium EDTA (15 mL)/ice and extracted with ethyl acetate (15 mL). The aqueous layer was saturated with NaCl and extracted further with ethyl acetate (15 mL×3). The combined organic layers were washed with aqueous sodium EDTA (15 mL) and brine (15 mL). The organic layer was dried over anhydrous Na2SO4. After filtration, the solvent was removed in vacuum and the residue was purified by silica gel chromatography to afford the title compound (150 mg, 38%). LCMS: 454.1 [M+H]⁺; ¹H NMR (400 MHz, CDCl₃) δ 8.49 (s, 1H), 7.77 (s, 1H), 6.77 (t, J=55.3 Hz, 1H), 6.14 (p, J=9.8 Hz, 1H), 5.62 (br s, 0.5H), 5.31 (br s, 0.5H), 4.15-4.03 (m, 1H), 3.90-3.70 (m, 2H), 3.15-2.75 (m, 2H), 2.85 (s, 3H), 2.31-2.06 (m, 3H), 2.16-2.06 (m, 2H), 1.80-1.60 (m, 2H), 1.45-1.35 (m, 2H), 1.30-1.20 (m, 1H), 0.95-0.82 (m, 2H).

### Example—S157: Synthesis of 8-(bicyclo[0.1.l]pentan-1-yl)-6-(difluoromethyl)-2-((1-(methylsulfonyl)piperidin-4-yl)amino)pyrido[2,3-d]pyrimidin-7(8H)-one (Compound 390)

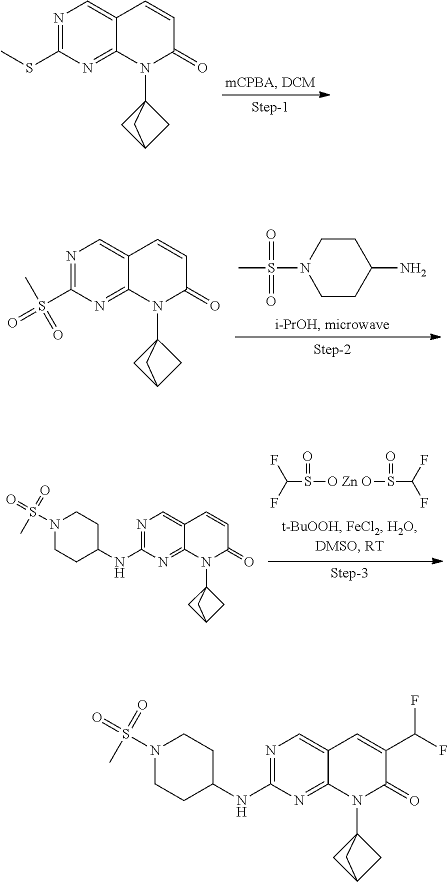

Step—1: Synthesis of 8-(bicyclo[1.1.1]pentan-1-yl)-2-(methylsulfonyl)pyrido[2,3-d]pyrimidin-7(8H)-one: To a solution of 8-(bicyclo[1.1.1]pentan-1-yl)-2-(methylthio)pyrido [2,3-d]pyrimidin-7(8H)-one (1.8 g, 6.94 mmol, 1.0 eq) in dichloromethane (60 mL) was added m-CPBA (2.98 g, 17.34 mmol) portion wise. The mixture was stirred at RT for 1 h. After that the mixture was quenched with saturated aqueous Na2SO3 (40 mL). The organic layer was washed with saturated aqueous Na2CO₃ (40 mL×2) and brine (40 mL). The organic layer was dried over anhydrous Na2SO₄. After filtration, the solution was concentrated under vacuum and the obtained solid was purified by silica gel chromatography to afford the title compound (1.3 g, 70%). LCMS: 292.0 [M+H]⁺

Step—2: Synthesis of 8-(bicyclo[1.1.l]pentan-1-yl)-2-((1-(methylsulfonyl)piperidin-4-yl)amino)pyrido[2,3-d]pyrimidin-7(8H)-one: To a sealed microwave tube was added a solution of 8-(bicyclo[1.1.1]pentan-1-yl)-2-(methylsulfonyl)pyrido[2,3-d]pyrimidin-7(8H)-one (900 mg, 3.09 mmol, 1.0 eq), 1-(methylsulfonyl)piperidin-4-amine (660 mg, 3.71 mmol, 1.2 eq) in i-PrOH (18 mL), and the reaction was heated at 130° C. under microwave for 1 h. The solvent was concentrated under vacuum. And the residue was re-dissolved in 20 mL of CH₂Cl₂ and washed with water (20 mL) for three times. The organic layer was dried over anhydrous Na2SO4. After filtration, the solvent was concentrated under vacuum and the obtained solid was purified by silica gel chromatography to afford the title compound (480 mg, 50%). LCMS: 390.0 [M+H]⁺

Step—3: Synthesis of 8-(bicyclo[1.1.1]pentan-1-yl)-6-(difluoromethyl)-2-((1-(methylsulfonyl)piperidin-4-yl)amino)pyrido[2,3-d]pyrimidin-7(8H)-one: To a solution of Zinc difluoromethanesulfinate (1083 mg, 3.69 mmol, 3.0 eq) and FeCl₂.4H₂O (122 mg, 0.62 mmol, 0.5 eq) in water (3 mL) was added dropwise to a solution of 8-(bicyclo[1.1.1]pentan-1-yl)-2-((1-(methylsulfonyl)piperidin-4-yl)amino)pyrido[2,3-d]pyrimidin-7(8H)-one (480 mg, 1.23 mmol, 1.0 eq) and TFA (140 mg, 1.23 mmol, 1.0 eq) in DMSO (40 mL) at RT. The resulting mixture was treated with TBHP (5-6N, 0.24 mL) and stirred at RT for 19 h. A second portion of TBHP (0.24 mL) was added and stirring continued for 3 h. A third portion of TBHP (0.24 mL) was added and stirring at RT for 45 min and then Zinc difluoromethanesulfinate (361 mg, 1.23 mmol, 1.0 eq) was added. The mixture was stirred at RT for another 8 h. The reaction solution was poured into a mixture of 10% aqueous sodium EDTA (15 mL)/ice and extracted with ethyl acetate (15 mL). The aqueous layer was saturated with NaCl and extracted further with ethyl acetate (15 mL×3). The combined organic layers were washed with aqueous sodium EDTA (15 mL) and brine (15 mL). The organic layer was dried over anhydrous Na2SO4. After filtration, the solvent was removed in vacuum and the residue was purified using Prep-TLC to afford the title compound (90 mg, 18%). LCMS: 440.0 [M+H]⁺; ¹H NMR (400 MHz, CDCl₃): δ 8.45 (s, 1H), 7.75 (s, 1H), 6.76 (t, J=55.3 Hz, 1H), 5.60 (s, 0.6H), 5.35 (s, 0.2H), 5.35 (s, 0.2H), 4.03 (br s, 1H), 3.95-3.75 (m, 2H), 2.96 (t, J=13.5 Hz, 2H), 2.85 (s, 3H), 2.70-2.60 (m, 7H), 2.25-2.11 (m, 2H), 1.75-1.68 (m, 2H).

### Example—S158: Synthesis of Racemic 8-((S,2S,4R)-bicyclo[2.2.1]heptan-2-yl)-6-(difluoromethyl)-2-((1-(methylsulfonyl)piperidin-4-yl)amino)pyrido[2,3-d]pyrimidin-7(8H)-one (Compound 401)

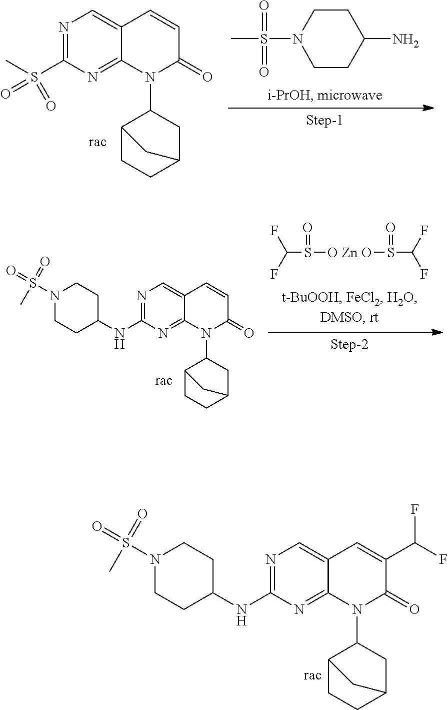

Step—1: Synthesis of Rac-8-((1S,2S,4R)-bicyclo[2.2.1]heptan-2-yl)-2-((1-(methylsulfonyl)piperidin-4-yl)amino)pyrido[2,3-d]pyrimidin-7(8H)-one: To a sealed microwave tube was added a solution of Racemic 8-((1S,2S,4R)-bicyclo[2.2.1]heptan-2-yl)-2-(methylsulfonyl)pyrido[2,3-d]pyrimidin-7(8H)-one (1 g, 3.14 mmol, 1.0 eq), 1-(methylsulfonyl)piperidin-4-amine (838 mg, 4.71 mmol, 1.5 eq) in i-PrOH (15 mL), and the reaction was heated at 130° C. under microwave for 1 h. The solvent was removed in vacuum and the residue was dissolved in CH₂Cl₂ (50 mL). The mixture was washed with H₂O (50 mL) and brine (50 mL). The organic phase was dried over anhydrous Na2SO4. After filtration, the solvent was removed in vacuum and the residue was purified by silica gel chromatography to afford the title compound (1 g, 76%). LCMS: 418.1 [M+H]⁺

Step—2: Synthesis of Rac-8-((1S,2S,4R)-bicyclo[2.2.1]heptan-2-yl)-6-(difluoromethyl)-2-((1-(methylsulfonyl)piperidin-4-yl)amino)pyrido[2,3-d]pyrimidin-7(8H)-one: To a solution of Zinc difluoromethanesulfinate (2.1 g, 2.38 mmol, 1.0 eq) and FeC₂.4H₂O (237 mg, 1.19 mmol, 0.5 eq) in water (7 mL) was added dropwise to a solution of Rac-8-((1S,2S,4R)-bicyclo[2.2.1]heptan-2-yl)-2-((1-(methylsulfonyl)piperidin-4-yl)amino)pyrido[2,3-d]pyrimidin-7(8H)-one (990 mg, 2.38 mmol, 1.0 eq) and TFA (271 mg, 2.38 mmol, 1.0 eq) in DMSO (40 mL) under N2 atmosphere at RT. The resulting mixture was treated with TBHP (5-6N, 0.5 mL). Stirring was continued at RT for 19 h. A second portion of TBHP (0.5 mL) was added and stirring continued for 3 h. A third portion of TBHP (0.5 mL) was added and stirring at RT for 45 min. The reaction solution was poured into a mixture of 10% aqueous sodium EDTA (40 mL)/ice and extracted with ethyl acetate (40 mL). The aqueous layer was saturated with NaCl and extracted further with ethyl acetate (40 mL×3). The combined organic layers were washed with aqueous sodium EDTA (40 mL) and brine (40 mL). The organic layer was dried over anhydrous Na2SO4. After filtration, the solvent was removed in vacuum and the residue was purified by silica gel chromatography to afford the title compound (280 mg, 25%). LCMS: 468.1 [M+H]+¹H NMR (400 MHz, CDCl₃) δ 8.47 (s, 1H), 7.77 (s, 1H), 6.77 (t, J=55.4 Hz, 1H), 5.65 (s, 0.7H), 5.32 (s, 0.3H), 5.25-5.10 (m, 1H), 4.20-3.90 (m, 1H), 3.85-3.70 (m, 2H), 3.00-2.85 (m, 2H), 2.85 (s, 3H), 2.65-2.50 (m, 1H), 2.55-2.40 (m, 2H), 2.30-2.10 (m, 2H), 1.75-1.60 (m, 6H), 1.35-1.20 (m, 3H).

### Example—S159: Synthesis of 8-((1R,3R,5S)-bicyclo[3.1.0]hexan-3-yl)-6-(difluoromethyl)-2-((1-((1-methyl-1H-pyrazol-3-yl)sulfonyl)piperidin-4-yl)amino)pyrido[2,3-d]pyrimidin-7(8H)-one (Compound 393)

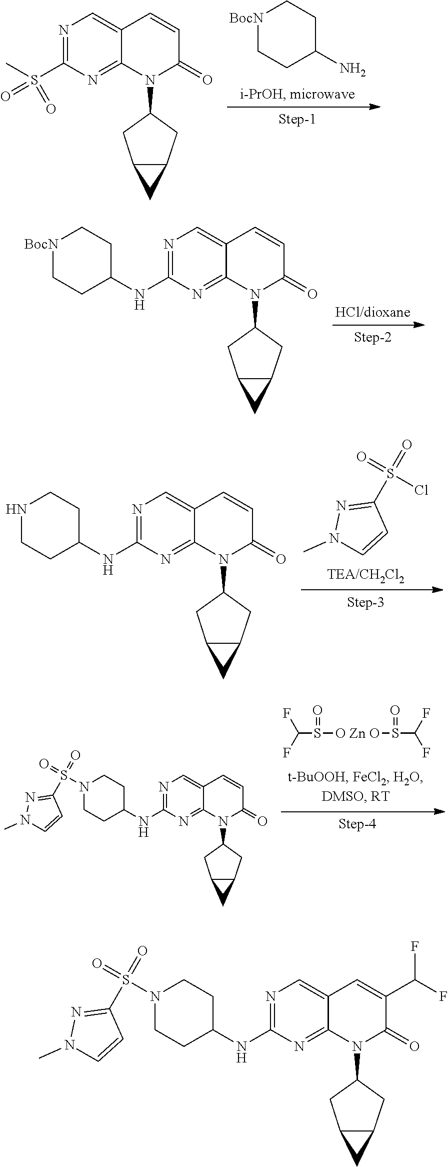

Step—1: Tert-butyl-4-((8-((1R,3R,5S)-bicyclo[3.1.0]hexan-3-yl)-7-oxo-7,8-dihydropyrido[2,3-d]pyrimidin-2-yl)amino)piperidine-1-carboxylate: To a sealed microwave tube was added a solution of 8-((1R,3R,5S)-bicyclo[3.1.0]hexan-3-yl)-2-(methylsulfonyl)pyrido[2,3-d]pyrimidin-7(8H)-one (380 mg, 1.25 mmol, 1.0 eq), tert-butyl 4-aminopiperidine-1-carboxylate (374 mg, 1.87 mmol, 1.5 eq) in i-PrOH (12 mL), and the reaction was heated at 130° C. under microwave for 1 h. The solvent was removed in vacuum and the residue was dissolved in CH₂Cl₂ (50 mL). The mixture was washed with H₂O (50 mL) and brine (50 mL). The organic phase was dried over anhydrous Na2SO₄. After filtrate, the solvent was removed in vacuum and the residue was purified by silica gel chromatography to afford the title compound (390 mg, 73%). LCMS: 426.3 [M+H]⁺

Step—2: Synthesis of 8-((1R,3R,5S)-bicyclo[3.1.0]hexan-3-yl)-2-(piperidin-4-ylamino)pyrido[2,3-d]pyrimidin-7(8H)-one: To a solution of Tert-butyl-4-((8-((1R,3R,5S)-bicyclo[3.1.0]hexan-3-yl)-7-oxo-7,8-dihydropyrido[2,3-d]pyrimidin-2-yl)amino)piperidine-1-carboxylate (390 mg, 0.92 mmol, 1.0 eq) in dioxane (5 mL) was added HCl/dioxane (4 M, 15 mL), the mixture was running at RT for 1 h. The solvent was removed in vacuum to give crude desired product (460 mg, crude) which was used for the next step without any further purification. LCMS: 326.2 [M+H]⁺

Step—3: Synthesis of 8-((1R,3R,5S)-bicyclo[3.1.0]hexan-3-yl)-2-((1-((1-methyl-1H-pyrazol-3-yl)sulfonyl)piperidin-4-yl)amino)pyrido[2,3-d]pyrimidin-7(8H)-one: To a solution of 8-((1R,3R,5S)-bicyclo[3.1.0]hexan-3-yl)-2-(piperidin-4-ylamino)pyrido[2,3-d]pyrimidin-7(8H)-one (460 mg, crude) and 1-methyl-1H-pyrazole-3-sulfonyl chloride (249 mg, 1.38 mmol) in CH₂Cl₂ (40 mL) was added TEA (279 mg, 2.76 mmol). The mixture was running at RT for 1 h. The mixture was washed with aqueous citric acid (10%, 30 mL), saturated NaHCO₃(30 mL) and brine (30 mL). The organic phase was dried over anhydrous Na2SO4, filtered and the solvent was removed in vacuum and the residue was purified by silica gel chromatography to afford the title compound (360 mg). LCMS: 470.1 [M+H]⁺

Step—4: Synthesis of 8-((1R,3R,5S)-bicyclo[3.1.0]hexan-3-yl)-6-(difluoromethyl)-2-((1-((1-methyl-1H-pyrazol-3-yl)sulfonyl)piperidin-4-yl)amino)pyrido[2,3-d]pyrimidin-7(8H)-one: To a solution of Zinc difluoromethanesulfinate (682 mg, 2.31 mmol, 3.0 eq) and FeCl₂.4H₂O (77 mg, 0.39 mmol, 0.5 eq) in water (3 mL) was added dropwise to a solution of 8-((1R,3R,5S)-bicyclo[3.1.0]hexan-3-yl)-2-((1-((1-methyl-1H-pyrazol-3-yl)sulfonyl)piperidin-4-yl)amino)pyrido[2,3-d]pyrimidin-7(8H)-one (360 mg, 0.77 mmol, 1.0 eq) and TFA (88 mg, 0.77 mmol, 1.0 eq) in DMSO (14 mL) under N2 atmosphere at RT. The resulting mixture was treated with TBHP (5-6N, 0.15 mL). Stirring was continued at RT for 19 h. A second portion of TBHP (0.15 mL) was added and stirring continued for 3 h. A third portion of TBHP (0.15 mL) was added and stirring at RT for 45 min. The reaction solution was poured into a mixture of 10% aqueous sodium EDTA (15 mL)/ice and extracted with ethyl acetate (15 mL). The aqueous layer was saturated with NaCl and extracted further with ethyl acetate (15 mL×3). The combined organic layers were washed with aqueous sodium EDTA (15 mL) and brine (15 mL). The organic layer was dried over anhydrous Na2SO4. After filtration, the solvent was removed in vacuum and the residue was purified by silica gel chromatography and Prep-HPLC to afford the title compound (26 mg, 6%). LCMS: 520.2 [M+H]⁺; ¹H NMR (400 MHz, CDCl₃) δ 8.45 (s, 1H), 7.75 (s, 1H), 7.47 (d, J=1.9 Hz, 1H), 6.76 (t, J=55.3 Hz, 1H), 6.68 (d, J=1.9 Hz, 1H), 6.20-6.01 (m, 1H), 5.60 (s, 0.5H), 5.30 (s, 0.5H), 4.02 (s, 3H), 3.93-3.80 (m, 1H), 3.76-3.60 (m, 1H), 3.13-2.92 (m, 1H), 2.85-2.67 (m, 1H), 2.27-2.21 (m, 2H), 2.18-2.06 (m, 3H), 1.82-1.59 (m, 3H), 1.37-1.26 (m, 3H), 0.95-0.77 (m, 2H).

### Example—S160: Synthesis of 8-(bicyclo[1.1.1]pentan-1-yl)-6-(difluoromethyl)-2-((1-((1-methyl-1H-pyrazol-3-yl)sulfonyl)piperidin-4-yl)amino)pyrido[2,3-d]pyrimidin-7(8H)-one (Compound 394)

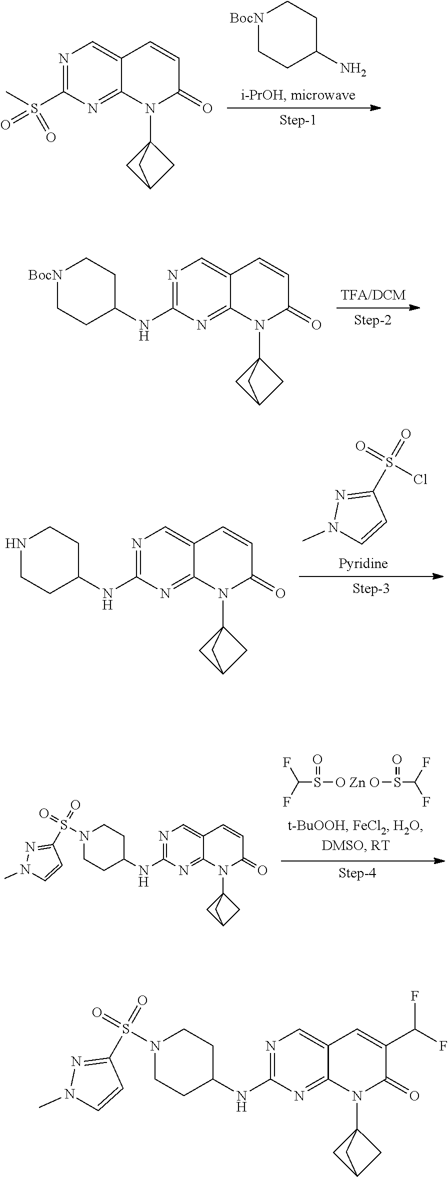

Step—1: Synthesis of Tert-butyl-4-((8-(bicyclo[1.1.]pentan-1-yl)-7-oxo-7,8-dihydropyrido[2,3-d]pyrimidin-2-yl) amino)piperidine-1-carboxylate: To a sealed microwave tube was added a solution of 8-(bicyclo[1.1.1]pentan-1-yl)-2-(methylsulfonyl)pyrido[2,3-d]pyrimidin-7(8H)-one (900 mg, 3.09 mmol), tert-butyl 4-aminopiperidine-1-carboxylate (742 mg, 3.712 mmol) in i-PrOH (20 mL), and the reaction was heated at 130° C. under microwave for 1 h. The solvent was removed in vacuum and the residue was dissolved in dichloromethane (50 mL). The mixture was washed with H₂O (50 mL) and brine (50 mL). The organic phase was dried over anhydrous Na2SO₄, filtered and the solvent was evaporated under vacuum and the obtained solid was purified by silica gel chromatography to afford the title compound (600 mg, 50%). LCMS: 412.0 [M+H]⁺

Step—2: Synthesis of 8-(bicyclo[1.1.1]pentan-1-yl)-2-(piperidin-4-ylamino)pyrido[2,3-d]pyrimidin-7(8H)-one: To a solution of Tert-butyl-4-((8-(bicyclo[1.1.1]pentan-1-yl)-7-oxo-7,8-dihydropyrido[2,3-d]pyrimidin-2-yl) amino)piperidine-1-carboxylate (600 mg, 1.46 mmol) in dichloromethane (10 mL) was added HCl/dioxane (4 M solution, 3 mL) at RT. The mixture was stirred at RT for 1 h. The mixture was evaporated under vacuum to give title compound which was directly used in the next step without any purification. LCMS: 312.0 [M+H]⁺

Step—3: Synthesis of 8-(bicyclo[1.1.1]pentan-1-yl)-2-((1-((1-methyl-1H-pyrazol-3-yl)sulfonyl)piperidin-4-yl)amino)pyrido[2,3-d]pyrimidin-7(8H)-one: To a solution of 8-(bicyclo[1.1.1]pentan-1-yl)-2-(piperidin-4-ylamino)pyrido[2,3-d]pyrimidin-7(8H)-one (430 mg, crude) in dichloromethane (10 mL) was added 1-methyl-1H-pyrazole-3-sulfonyl chloride (296 mg, 1.65 mmol) and TEA (167 mg, 1.65 mmol) at RT and stirred at RT for 3 h. The mixture was washed with aqueous citric acid (10%, 20 mL), saturated NaHCO₃(20 mL) and brine (20 mL). The organic phase was dried over anhydrous Na2SO4, filtered and the solvent was evaporated under vacuum and the residue was purified by silica gel chromatography to afford the title compound (540 mg, 80%). LCMS: 456.0 [M+H]⁺

Step—4: Synthesis of 8-(bicyclo[1.1.1]pentan-1-yl)-6-(difluoromethyl)-2-((1-((1-methyl-1H-pyrazol-3-yl)sulfonyl)piperidin-4-yl)amino)pyrido[2,3-d]pyrimidin-7(8H)-one: To a solution of Zinc difluoromethanesulfinate (1.044 g, 3.55 mmol) and FeC₂.4H₂O (117 mg, 0.59 mmol) in water (5 mL) was added dropwise to a solution of 8-(bicyclo[1.1.1]pentan-1-yl)-2-((1-((1-methyl-1H-pyrazol-3-yl)sulfonyl)piperidin-4-yl)amino)pyrido[2,3-d]pyrimidin-7(8H)-one (540 mg, 1.18 mmol) and TFA (135 mg, 1.184 mmol) in DMSO (30 mL) at RT. The resulting mixture was treated with TBHP (5-6N, 0.22 mL). Stirring was continued at RT for 19 h. A second portion of TBHP (0.24 mL) was added and stirring continued for 3 h. A third portion of TBHP (0.24 mL) was added and stirring at RT for 45 min and then Zinc difluoromethanesulfinate (348 mg, 1.18 mmol) was added. The mixture was stirred at RT for another 8 h. The reaction solution was poured into a mixture of 10% aqueous sodium EDTA (15 mL)/ice and extracted with ethyl acetate (15 mL). The aqueous layer was saturated with NaCl and extracted further with ethyl acetate (15 mL×3). The combined organic layers were washed with aqueous sodium EDTA (15 mL) and brine (15 mL). The organic layer was dried over anhydrous Na2SO4. After filtration, the solvent was removed in vacuum and the residue was purified by Prep-TLC to give afford the title compound (105 mg, 22%). LCMS: 506.0 [M+H]⁺; ¹H NMR (400 MHz, CDCl₃): δ 8.43 (s, 1H), 7.73 (s, 1H), 7.48 (d, J=2.0 Hz, 1H), 6.75 (t, J=55.3 Hz, 1H), 6.68 (d, J=2.2 Hz, 1H), 5.57 (d, J=7.1 Hz, 0.7H), 5.24 (br s, 0.3H), 4.02 (s, 3H), 3.97-3.84 (m, 2H), 3.72 (d, J=11.3 Hz, 1H), 2.94 (t, J=10.3 Hz, 1H), 2.72-2.66 (m, 8H), 2.21-2.10 (m, 2H), 1.78-1.67 (m, 2H).

### Example—S161: Synthesis of 8-((S,2S,4R)-bicyclo[2.2.1]heptan-2-yl)-6-(difluoromethyl)-2-((1-((1-methyl-1H-pyrazol-3-yl)sulfonyl)piperidin-4-yl)amino)pyrido[2,3-d]pyrimidin-7(8H)-one (Compound 395) & Synthesis of 8-((1R,2R,4S)-bicyclo[2.2.1]heptan-2-yl)-6-(difluoromethyl)-2-((1-((1-methyl-1H-pyrazol-3-yl)sulfonyl)piperidin-4-yl)amino)pyrido[2,3-d]pyrimidin-7(8H)-one (Compound 396)

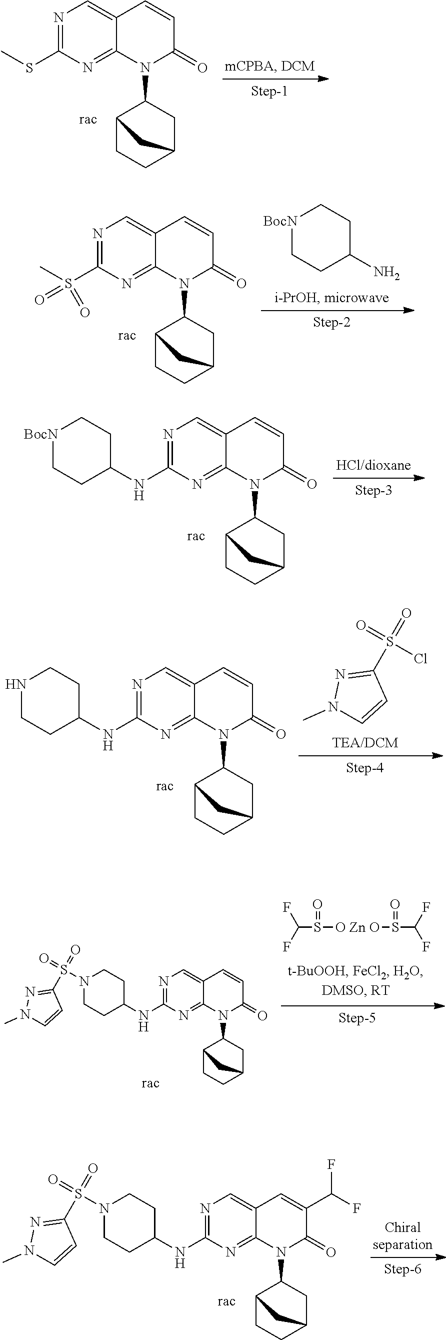

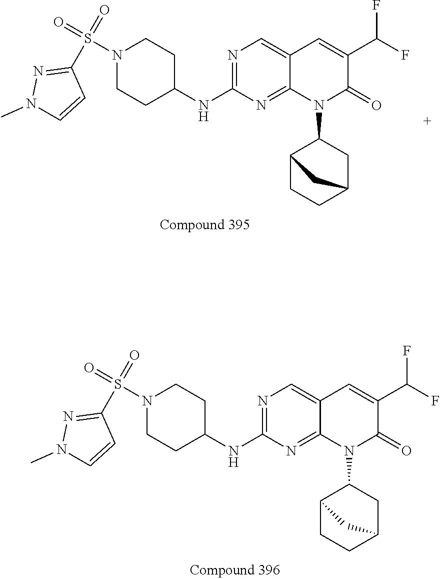 [SMILES: Cn1ccc(S(=O)(=O)N2CCC(Nc3ncc4cc(C(F)F)c(=O)n([C@H]5C[C@@H]6CC[C@H]5C6)c4n3)CC2)n1 ; Cn1ccc(S(=O)(=O)N2CCC(Nc3ncc4cc(C(F)F)c(=O)n([C@@H]5C[C@H]6CC[C@@H]5C6)c4n3)CC2)n1]

Step—1: Synthesis of Rac-8-((1S,2S,4R)-bicyclo[2.2.1]heptan-2-yl)-2-(methylsulfonyl)pyrido[2,3-d]pyrimidin-7(8H)-one (1): To a solution of racemic 8-((1S,2S,4R)-bicyclo[2.2.1]heptan-2-yl)-2-(methylthio)pyrido[2,3-d]pyrimidin-7(8H)-one (3.16 g, 11 mmol) in CH₂Cl₂ (200 mL) was added mCPBA (5.57 g, 27.5 mmol) portion wise. The mixture was running at RT for 2 h. The mixture was quenched with saturated Na2SO3 (200 mL). The organic layer was washed with saturated Na2CO₃ (200 mL×2) and brine (200 mL). The organic layer was dried over anhydrous Na2SO4. After filtrate, the solvent was removed in vacuum and the residue was purified by silica gel chromatography to afford the title compound (2.81 g, 80%). LCMS: 319.9 [M+H]⁺

Step 2: Synthesis of Rac-tert-butyl-4-((8-((1S,2S,4R)-bicyclo[2.2.1]heptan-2-yl)-7-oxo-7,8-dihydropyrido[2,3-d]pyrimidin-2-yl)amino)piperidine-1-carboxylate: To a sealed microwave tube was added a solution of Rac-8-((1S,2S,4R)-bicyclo[2.2.1]heptan-2-yl)-2-(methylsulfonyl)pyrido[2,3-d]pyrimidin-7(8H)-one (1.64 g, 5.14 mmol), tert-butyl 4-aminopiperidine-1-carboxylate (2.05 g, 10.28 mmol) in i-PrOH (15 mL), and the reaction was heated at 130° C. under microwave for 1 h. The solvent was removed in vacuum and the residue was dissolved in CH₂Cl₂ (50 mL). The mixture was washed with H₂O (50 mL) and brine (50 mL). The organic phase was dried over anhydrous Na2SO₄. After filtrate, the solvent was removed in vacuum and the residue was purified by silica gel chromatography to afford the title compound (1.6 g, 70%). LCMS: 439.9 [M+H]⁺

Step—3: Synthesis of Rac-8-((1S,2S,4R)-bicyclo[2.2.1]heptan-2-yl)-2-(piperidin-4-ylamino)pyrido[2,3-d]pyrimidin-7(8H)-one: To a solution of Rac-tert-butyl-4-((8-((1S,2S,4R)-bicyclo[2.2.1]heptan-2-yl)-7-oxo-7,8-dihydropyrido[2,3-d]pyrimidin-2-yl)amino)piperidine-1-carboxylate (1.6 g, 3.64 mmol) in dioxane (8 mL) was added HCl/dioxane (4 M, 24 mL), the mixture was running at RT for 1 h. The solvent was removed in vacuum to furnish the title compound (1.4 g, crude) which was used for the next step without further purification. LCMS: 340.2 [M+H]⁺

Step—4: Synthesis of Rac-8-((1S,2S,4R)-bicyclo[2.2.1]heptan-2-yl)-2-((1-((1-methyl-1H-pyrazol-3-yl)sulfonyl)piperidin-4-yl)amino)pyrido[2,3-d]pyrimidin-7(8H)-one: To a solution of Rac-8-((1S,2S,4R)-bicyclo[2.2.1]heptan-2-yl)-2-(piperidin-4-ylamino)pyrido[2,3-d]pyrimidin-7(8H)-one (1.4 g, crude) and 1-methyl-1H-pyrazole-3-sulfonyl chloride (959 mg, 5.3 mmol) in CH₂Cl₂ (40 mL) was added TEA (1.8 g, 17.8 mmol). The mixture was stirring at RT for 1 h. The mixture was washed with aqueous citric acid (10%, 30 mL), saturated NaHCO₃(30 mL) and brine (30 mL). The organic phase was dried over anhydrous Na2SO₄. After filtration, the solvent was removed in vacuum and the residue was purified by silica gel chromatography to afford the title compound (1.3 g, 73% for 2 steps). LCMS: 484.1 [M+H]⁺

Step—5: Synthesis of 8-((1S,2S,4R)-bicyclo[2.2.1]heptan-2-yl)-6-(difluoromethyl)-2-((1-((1-methyl-1H-pyrazol-3-yl)sulfonyl)piperidin-4-yl)amino)pyrido[2,3-d]pyrimidin-7(8H)-one & Synthesis of 8-((1R,2R,4S)-bicyclo[2.2.1]heptan-2-yl)-6-(difluoromethyl)-2-((1-((1-methyl-1H-pyrazol-3-yl)sulfonyl)piperidin-4-yl)amino)pyrido[2,3-d]pyrimidin-7(8H)-one: To a solution of Zinc difluoromethanesulfinate (2.36 g, 8.01 mmol) and FeC₂.4H₂O (265 mg, 1.34 mmol) in water (7 mL) was added dropwise to a solution of Rac-8-((1S,2S,4R)-bicyclo[2.2.1]heptan-2-yl)-2-((1-((1-methyl-1H-pyrazol-3-yl)sulfonyl)piperidin-4-yl)amino)pyrido[2,3-d]pyrimidin-7(8H)-one (1.29 g, 2.67 mmol) and TFA (304 mg, 2.67 mmol) in DMSO (40 mL) under N2 atmosphere at RT. The resulting mixture was treated with TBHP (5-6N, 0.5 mL). Stirring was continued at RT for 19 h. A second portion of TBHP (0.5 mL) was added and stirring continued for 3 h. A third portion of TBHP (0.5 mL) was added and stirring at RT for 45 min. The reaction solution was poured into a mixture of 10% aqueous sodium EDTA (40 mL)/ice and extracted with ethyl acetate (40 mL). The aqueous layer was saturated with NaCl and extracted further with ethyl acetate (40 mL×3). The combined organic layers were washed with aqueous sodium EDTA (40 mL) and brine (40 mL). The organic layer was dried over anhydrous Na2SO4. After filtration, the solvent was removed in vacuum and the residue was purified by silica gel chromatography to afford the title compound as a racemate (300 mg, 21%), LCMS: 534.1 [M+H]+ which was further purified by chiral SFC to afford the desired title compounds.

Compound 395: 97 mg; ¹H NMR (400 MHz, CDCl3) δ 8.44 (s, 1H), 7.75 (s, 1H), 7.47 (d, J=2.2 Hz, 1H), 6.76 (t, J=55.6 Hz, 1H), 6.68 (d, J=2.4 Hz, 1H), 5.67 (br s, 0.7H), 5.30 (br s, 0.3H), 5.17-5.13 (m, 1H), 4.01 (s, 3H), 3.97-3.85 (m, 1H), 3.82-3.70 (m, 1H), 2.91-2.74 (m, 2H), 2.65-2.55 (m, 1H), 2.53-2.49 (m, 1H), 2.47-2.38 (m, 1H), 2.25-2.10 (m, 2H), 1.85-1.56 (m, 7H), 1.35-1.20 (m, 3H).

Compound 396: 122 mg; ¹H NMR (400 MHz, CDCl₃) δ 8.44 (s, 1H), 7.75 (s, 1H), 7.47 (d, J=2.3 Hz, 1H), 6.76 (t, J=55.6 Hz, 1H), 6.68 (d, J=2.4 Hz, 1H), 5.90-5.10 (m, 2H), 4.01 (s, 3H), 4.00-3.84 (m, 1H), 3.82-3.70 (m, 1H), 3.00-2.68 (m, 2H), 2.64-2.54 (m, 1H), 2.53-2.48 (m, 1H), 2.46-2.36 (m, 1H), 2.23-2.10 (m, 2H), 1.85-1.50 (m, 7H), 1.36-1.20 (m, 3H).

It is understood that compounds from the Table-1 are synthesized using the General Synthetic Schemes 1 to 4 or according to the experimental details as exemplified in Examples S1-S107 using the appropriate starting materials and reagents. In some embodiments, the compounds from Table 1 are synthesized using the General Synthetic Schemes 1 to 4 or according to the experimental details as exemplified in Examples S1-S161.

### Biological Examples

### Example B1. In Vitro Kinase Inhibition IC₅₀ Determination

IC₅₀ values of compounds against CDK4 and CDK6 were determined by luminescence using retinoblastoma as substrate. Kinase assays were performed in kinase buffer (#PV6135, Invitrogen Life Technologies, Grand Island, N.Y.) where total reaction volume was 30 μL/well in 96-well half area white plates (#3693, Costar). One microliter of 25×test compounds at specific concentrations (e.g., final concentration range: 0.1 nM-200 nM) was mixed with 10 μL of 2.5×kinase (5 nM, CDK4 #PR8064A and CDK6 #PR8422B, Invitrogen) solution and 14 μL of 4× mixed solution with retinoblastoma (1 μM, #12-439, EMD Millipore, Hayward, Calif.) and ATP (25 μM, #V7038, Promega, Madison, Wis.). The plates were covered and incubated for 2H at room temperature. At the end of incubation, 25 μL of stop solution—ADP Glo reagent (#V7002, Promega) was added. After incubation for 45 min at room temperature, 50 μL of detection reagent (##V7002, Promega) was added. Readings were taken at 15 min and 45 min incubation after detection reagent was added in a Synergy Neo Plate reader (BioTek, Winooski, Vt.) at single excitation of 340 nm and Dual emission at 495 nm and 520 nm respectively. The following equations were used in the CDK4 and CDK6 assay data analysis. Percent inhibition (100-% activity) was fitted to the “four-parameter logistic model” in XLfit for determination of IC₅₀ values.

Percent conversion of enzyme=100−{(RLU_{No Drug-No enzyme}*100)/RLU_{No drug+Enzyme}} Equation 1:

Percent conversion at each data point=100−{(RLU_{Average(Drug+enzyme)}*100)!RLU_{No drug+Enzyme}} Equation 2:

Percent Inhibition=100*(% Conversion_{each data point}/% Conversion_{Enzyme}) Equation 3:

IC₅₀ values of compounds against CDK1 (cyclin B) were determined by Z′-LYTE™ These screening assays were performed at Invitrogen Life Technologies (Grand Island, N.Y.) on a low volume NBS, black 384-well plate (#4514, Corning). 0.1 μL of 100×test compound in 100% DMSO (at specific solutions) was mixed with 2.4 μL of Kinase Buffer (50 mM HEPES pH 7.5, 0.01% BRIJ-35, 10 mM MgCl₂, 1 mM EGTA), 5 μL of 2× Kinase (3.5-46.4 ng CDK1/cyclin B)/Peptide (2 μM Ser/Thr 18), and 2.5 μL of 4×ATP solution (34 μM). The plates were shaken for 30 seconds, and incubated for 60 minutes at room temperature. Development Reagent Solution (5 μL of 1:1024 dilution) was added to the plates followed with another 30-second plate shake, and the plates were further incubated at room temperature for one hour. The plates were read on fluorescence plate reader with Dual emission at 445 nm and 520 nm.

IC₅₀ values of compounds against CDK2 (cyclin A) were determined by Z′-LYTE™ These screening assays were performed at Invitrogen Life Technologies (Grand Island, N.Y.) on a low volume NBS, black 384-well plate (#4514, Corning). 0.1 μL of 100×test compound in 100% DMSO (at specific solutions) was mixed with 2.4 μL of Kinase Buffer (50 mM HEPES pH 7.5, 0.01% BRIJ-35, 10 mM MgCl₂, 1 mM EGTA), 5 μL of 2× Kinase (1.22-10.3 ng CDK2/cyclin A)/Peptide (2 μM Ser/Thr 12), and 2.5 μL of 4×ATP solution (31 μM). The plates were shaken for 30 seconds, and incubated for 60 minutes at room temperature. Development Reagent Solution (5 μL of 1:1024 dilution) was added to the plates followed with another 30-second plate shake and the plates were further incubated at room temperature for one hour. The plates were read on fluorescence plate reader with Dual emission at 445 nm and 520 nm.

IC₅₀ values of compounds against CDK5 (p25) are determined by Z′-LYTE™. These screening assays are performed at Invitrogen Life Technologies (Grand Island, N.Y.) on a low volume NBS, black 384-well plate (#4514, Corning). 0.1 μL of 100×test compound in 100% DMSO (at specific solutions) is mixed with 2.4 μL of Kinase Buffer (50 mM HEPES pH 7.5, 0.01% BRIJ-35, 10 mM MgCl₂, 1 mM EGTA), 5 μL of 2× Kinase (0.18-2 ng CDK5/p25)/Peptide (2 μM Ser/Thr 12), and 2.5 μL of 4×ATP solution (17 μM). The plates are shaken for 30 seconds, and incubated for 60 minutes at room temperature. Development Reagent Solution (5 μL of 1:4096 dilution) is added to the plates followed with another 30-second plate shake and the plates are further incubated at room temperature for one hour. The plates are read on fluorescence plate reader with Dual emission at 445 nm and 520 nm.

The following equations were used for Z′-LYTE™ Screening Assay Data Analysis. Percent inhibition (100-% activity) was fitted to the “four-parameter logistic model” in XLfit for determination of IC₅₀ values.

| | Equation |
|---|---|
| Correction for Background Fluorescence | FI ₛₐₘₚₗₑ − FI _{TCFI Ctl} |
| Emission Ratio (using values corrected for background | $\frac{Coumarin Emission ( 445 nm ) }{Flourescein Emission ( 520 nm )}$ |
| fluorescence) | |
| % Phosphorylation (% Phos) | $\{ 1 - \frac{( Emission Ratio \times {F}_{100 \%} ) - {C}_{100 \%}}{( {C}_{0 \%} - {C}_{100 \%} ) + [ Emission Ratio \times ( {F}_{100 \%} - {F}_{0 \%} ) ]} \} * 100$ |
| % Inhibition | $\{ 1 - \frac{\% {Phos }_{Sample}}{ \% {Phos }_{0 \% \text{?} }} \} * 100$ |
| Z^{′} (using Emission Ratio values) | $1 - \frac{3 * {Stdev}_{ 0 \% Phos Ctl } \text{?} 3 * {Stdev }_{0 \% Inhibition}}{{Mean }_{0 \% Phos Ctl} - {Mean}_{ 0 \% Inhibition}}$ |
| Difference Between Data Points | \|% Inhibition _{Point 1} -% Inhibition _{Point 2}\| |
| (single point only) | |
| Development Reaction Interference (DRI) (no ATP control) | $\frac{Emission {Ratio }_{DRI Ctl}}{Emission {Ratio }_{0 \% Phos Ctl}}$ |
| Test Compound Fluorescence Interference (TCFI) | $\frac{{FI }_{TCFI Ctl}}{{FI}_{ 0 \% Inhibitor Ctl}}$ |
| (check both Coumarin and Fluorescein emissions) | |

FI = Fluorescence Intensity
C_{100%} = Average Coumarin emission signal of the 100% Phos. Control
C_{0%} = Average Coumarin emission signal of the 0% Phos. Control
F_{100%} = Average Fluorescein emission signal of the 100% Phos. Control
F_{0%} = Average Fluorescein emission signal of the 0% Phos. Control
DRI = Development Reaction Interference
TCFI = Compound Fluorescence Interference
$\text{?} \text{indicates text missing or illegible when filed}$

IC₅₀ values of compounds against CDK7 (cyclin H) are determined by Adapta™ Assay at Invitrogen Life Technologies (Grand Island, N.Y.) where total reaction volume is 10 μL/well in low volume, white 384-well plate (#4512, Corning). 0.100 μL of 100×test compound in 100% DMSO (at specific solutions) is mixed with 2.4 μL of HEPES (30 mM), 2.5 L of 4×ATP solution (153 μM) and 5 μL of 2×Substrate/Kinase mixture (the 2×CDK7/cyclin H/MNAT1/CDK7/9tide mixture is prepared in 50 mM HEPES pH 7.5, 0.01% BRIJ-35, 10 mM MgCl₂, 1 mM EGTA). The final 10 μL Kinase Reaction consists of 5-38.75 ng CDK7/cyclin H/MNAT1 and 200 μM CDK7/9tide in 32.5 mM HEPES pH 7.5, 0.005% BRIJ-35, 5 mM MgCl₂, 0.5 mM EGTA. The plates are shaken for 30 seconds, centrifuged for 1 min at 1000×g, and incubated for 60 minutes at room temperature. 5 μL of Detection Mix (prepared in TR-FRET Dilution Buffer; the Detection mix consists of EDTA (30 mM), Eu-anti-ADP antibody (6 nM) and ADP tracer, and contains the EC₆₀ concentration of tracer for 5-150 μM ATP) is added to the plates followed with another 30-second plate shake and centrifugation for 1 min at 1000×g, and the plates are further incubated at room temperature for one hour. The plates are read on fluorescence plate reader with Dual emission at 615 nm and 665 nm.

The following equations are used for Adapta™ Assay Data Analysis. The ATP/ADP standard curve is fit to model number 205 (sigmoidal dose-response model) in XLfit. The dose response curve is also curve fit to model number 205.

| | Equation |
|---|---|
| Emission Ratio | $\frac{AF 647 Emission ( 665 nm )}{Europium Emission ( 615 nm )}$ |
| % Conversion | $\{ \frac{{EC}_{50 SC}}{( \frac{{TOP}_{ SC} - {Bottom}_{ SC}}{Emission {Ratio}_{ Sample} - {Bottom}_{ SC}} ) - 1 \hat{} ( \frac{1}{{Hillslope }_{SC}} )} \} * 100$ |
| % Inhibition | $\{ 1 - \frac{\% {Conversion }_{Sample}}{\% Conversion \text{?}} \} * 100$ |
| Difference Between | \|% Inhibition _{Point 1} -% Inhibition _{Point 2}\| |
| Data Points | |
| (single point only) | |
| Test Compound | For each emission wavelength, fluorescence interference is flagged for |
| Interference | a compound well that is more than 20% outside the range of the |
| | controls. |
| Z^{′} (using Emission Ratio values) | $1 - \frac{3 * {Stdev}_{ 0 \% Conv Ctrl} + 3 * {Stdev }_{0 \% Inhibition}}{\vert {Mean }_{0 \% Conv Ctrl} - {Mean }_{0 \% Inhibition} \vert}$ |

* SC = Standard Curve
$\text{?} \text{indicates text missing or illegible when filed}$

IC₅₀ values of compounds against CDK2 (cyclin E1) were determined by LanthaScreen™ Eu Kinase Binding Assay at Invitrogen Life Technologies (Grand Island, N.Y.) where total reaction volume was 16 μL/well in low volume, white 384-well plate (#784207, Greiner). 0.16 μL of 100×test compound in 100% DMSO (at specific solutions) were mixed with 3.84 μL of Kinase Buffer (50 mM HEPES pH 7.5, 0.01% BRIJ-35, 10 mM MgCl₂, 1 mM EGTA), 8.0 μL of 2× Kinase (2.5 nM)/Antibody (Eu-anti-GST, 2 nM) Mixture and 4.0 μL of 4×Tracer (Tracer 236, 100 nM). The plates were shaken for 30 seconds, and incubated for 60 minutes at room temperature. The plates were read on fluorescence plate reader with Dual emission at 615 nm and 665 nm.

IC₅₀ values of compounds against CDK9 (cyclin K) were determined by LanthaScreen™ Eu Kinase Binding Assay at Invitrogen Life Technologies (Grand Island, N.Y.) where total reaction volume was 16 μL/well in low volume, white 384-well plate (#784207, Greiner). 0.16 μL of 100×test compound in 100% DMSO (at specific solutions) were mixed with 3.84 μL of Kinase Buffer (50 mM HEPES pH 7.5, 0.01% BRIJ-35, 10 mM MgCl₂, 1 mM EGTA), 8.0 μL of 2× Kinase (5 nM)/Antibody (Eu-anti-His, 2 nM) Mixture and 4.0 μL of 4×Tracer (Tracer 236, 100 nM). The plates were shaken for 30 seconds, and incubated for 60 minutes at room temperature. The plates were read on fluorescence plate reader with Dual emission at 615 nm and 665 nm.

IC₅₀ values of compounds against FMS kinase were determined by LanthaScreen™ Eu Kinase Binding Assay at Invitrogen (Life Technologies Grand Island, N.Y.) where total reaction volume was 10 μL in low-volume 384-well plates (#4511, Corning). Serially diluted compounds (3-fold) were incubated with kinase (1.25 nM) for 10 min, following which a mixture of ATP (10 μM) (#A1852, Sigma, St-Louis, Mo.) and fluorescent-PolyGT substrate (200 nM) (#PV3610, Invitrogen, Life Technologies Grand Island, N.Y.) was added and incubated in dark at room temperature for 1H. After 1H, 10 μL stop solution containing Terbium labeled antibody (4 nM) (#PV3529, Invitrogen, Life Technologies Grand Island, N.Y.) and EDTA (#E5134, Sigma, St-Louis, Mo.) (20 mM) in TR-FRET dilution buffer (#PV3574, Invitrogen, Life Technologies Grand Island, N.Y.) was added. Readings were taken in a Synergy Neo Plate reader (BioTek, Winooski) at single excitation of 340 nm and Dual emission at 495 nm and 520 nm respectively.

The following equations were used for LanthaScreen Eu Kinase Binding Assay Data Analysis. Percent inhibition (100-% activity) was fitted to the “four-parameter logistic model” in XLfit for determination of IC₅₀ values.

| | Equation |
|---|---|
| Emission Ratio (ER) | $\frac{AF 647 Emission ( 665 nm )}{Europium Emission ( 615 nm )}$ |
| % Displacement | $\{ \frac{{ER }_{0 \% Disp Ctrl} - {ER}_{ Sample}}{{ER }_{0 \% Disp Ctrl} - {ER }_{100 \% Disp Ctrl}} \} * 100$ |
| Difference Between | \|% Displacement _{Point 1} -% Displacement _{Point 2}\| |
| Data Points | |
| (single point only) | |
| Test Compound | For each emission wavelength, fluorescence interference is flagged for |
| Interference | a compound well that is more than 20% outside the range of the |
| | controls. |
| Z^{′} (using Emission Ratio values) | $1 - \frac{3 * {Stdev}_{ 0 \% Disp Ctrl} + 3 * {Stdev }_{100 \% Disp Ctrl}}{\vert {Mean }_{0 \% Disp Ctrl} - {Mean }_{100 \% Disp Ctrl} \vert}$ |

$\text{?} \text{indicates text missing or illegible when filed}$

IC₅₀ values of compounds against the PI3Kδ kinase were determined by an assay performed by Reaction Biology Corporation (Malvern, Pa.). Briefly, this assay was conducted in buffer (Tris-HCl 40 mM (pH7.5), Orthovanadate 3 mM, MgCl₂ 20 mM, DTT 2 mM, CHAPS 0.05%, DMSO 1%). PI3Kδ kinase was added to the reaction solution and mixed gently. The test compounds in 100% DMSO (at specific solutions) were mixed with the kinase reaction mixture to achieve the final compounds at pre-defined concentrations (e.g., range −0.5 nM to 100 μM) by Acoustic technology (Echo550; nanoliter range). After incubating for 10 min at room temp, ATP was added into the reaction mixture to initiate the reaction followed by a 30-min incubation at 30° C. After quenching the reaction with ADP-Glo reagent, the plates were incubated for 40 min. The Detection Mixture was added, and the plate was incubated for an additional 30 min. At the end of incubation, luminescence was measured. For data analysis, the luminescence was converted into μM ADP production based on ADP standard curves. The nonlinear regression to obtain the standard curve and IC₅₀ values was performed using GraphPad Prism (GraphPad Software, Inc., San Diego, Calif.).

IC₅₀ values of compounds against CDK12 (cyclin K) were determined by KinaseProfiler™ radiometric protein kinase assay at Eurofins Pharma Discovery (Dundee, UK). Compounds were prepared to 50× final assay concentration in 100% DMSO. This working stock of the compound was added to the assay well as the first component in each reaction. CDK12/Cyclin K was diluted in buffer (20 mM TRIS, 0.2 mM EDTA, 0.1%-mercaptoethanol, 0.01% Brij-35, 5% glycerol, mg/ml BSA) prior to addition to the reaction mix. CDK12/Cyclin K was incubated with 20 mM Tris/HCl pH 8.5, 0.2 mM EDTA, 300 μM SRSRSRSRSRSRSR, 10 mM Magnesium acetate and [γ-³³P-ATP] (specific activity and concentration as required). The reaction was initiated by the addition of the Mg/ATP mix. After incubation for 120 minutes at room temperature, the reaction was stopped by the addition of phosphoric acid to a concentration of 0.5%. 10 μl of the stopped reaction was spotted onto a P30 filtermat and washed four times for 4 minutes in 0.425% phosphoric acid and once in methanol prior to drying and scintillation counting. Results were calculated as a percentage of the mean kinase activity in positive control samples. Data were fitted in XLfit for determination of IC₅₀ values.

IC₅₀ values of compounds disclosed herein against the kinases listed above are given in Table 2 below.

TABLE 2

| Com- | CDK4/D1 | CDK6/D1 | CDK1/B | CDK2/A | CDK2/E | CDK9/K | CDK12/K | | |
|---|---|---|---|---|---|---|---|---|---|
| pound | IC₅₀ | IC₅₀ | IC₅₀ | IC₅₀ | IC₅₀ | IC₅₀ | IC₅₀ | FMS | PI3Kδ |
| No. | (nM) | (nM) | (nM) | (nM) | (nM) | (nM) | (nM) | IC₅₀ (nM) | IC₅₀ (nM) |
| 1 | 2 | 1 | ND | ND | ND | ND | ND | <300 | 389 |
| 2 | ND | ND | ND | ND | ND | ND | ND | 180 | 129 |
| 3 | 12 | 14 | ND | ND | ND | ND | ND | 35 | 130 |
| 4 | ND | ND | ND | ND | ND | ND | ND | 1 | 2730 |
| 5 | 212 | 48 | ND | ND | ND | ND | ND | 1 | 444 |
| 6 | 8 | 2 | >4646 | 873 | ND | ND | ND | <1 | 163 |
| 7 | ND | ND | ND | ND | ND | ND | ND | 8 | ND |
| 8 | ND | ND | ND | ND | ND | ND | ND | ND | 2460 |
| 9 | ND | ND | ND | ND | ND | ND | ND | <1 | 955 |
| 10 | 1 | 1 | ND | ND | ND | ND | ND | <1 | 310 |
| 11 | ND | ND | ND | ND | ND | ND | ND | 2.4 | 1030 |
| 12 | ND | ND | ND | ND | ND | ND | ND | 0.4 | 1330 |
| 13 | 3.5 | 1.5 | ND | ND | ND | ND | ND | <1 | 561 |
| 14 | ND | ND | ND | ND | ND | ND | ND | 2.5 | 976 |
| 15 | ND | ND | ND | ND | ND | ND | ND | 5.5 | 3730 |
| 16 | >200 | >200 | ND | ND | ND | ND | ND | 7 | ND |
| 17 | >200 | >200 | ND | ND | ND | ND | ND | 53 | ND |
| 18 | >200 | >200 | ND | ND | ND | ND | ND | 74 | ND |
| 19 | 35 | 38 | ND | ND | ND | ND | ND | <1 | ND |
| 20 | >200 | >200 | ND | ND | ND | ND | ND | 99 | ND |
| 21 | 11 | 2 | ND | ND | ND | ND | ND | 2.5 | 1010 |
| 22 | >200 | 65 | ND | ND | ND | ND | ND | <1 | 43 |
| 23 | 70.5 | 7 | ND | ND | ND | ND | ND | 53 | ND |
| 24 | >200 | 35 | ND | ND | ND | ND | ND | 5 | ND |
| 25 | >200 | 38 | ND | ND | ND | ND | ND | 13 | ND |
| 26 | 42 | 6 | ND | ND | ND | ND | ND | 6 | ND |
| 27 | 18 | 3.5 | ND | ND | ND | ND | ND | 3.5 | ND |
| 29 | 7 | 13 | 441 | 42 | ND | ND | ND | 2.5 | 552 |
| 30 | 4.5 | 9.5 | ND | ND | ND | ND | ND | 6 | ND |
| 31 | 5 | 9 | 481 | 32 | 74 | 20 | >10000 | 1 | 128 |
| 32 | 1 | 7 | ND | ND | ND | ND | ND | 3.5 | 251 |
| 33 | >200 | >200 | ND | ND | ND | ND | ND | 4.2 | 1657 |
| 34 | 6.5 | 9 | ND | ND | ND | ND | ND | 5.2 | 1229 |
| 35 | >200 | 91 | ND | ND | ND | ND | ND | 7 | 738 |
| 36 | 1 | 1 | ND | ND | ND | ND | ND | 6.5 | 1027 |
| 37 | 6.5 | 2.5 | ND | ND | ND | ND | ND | 5.8 | 64 |
| 38 | 1.5 | 1 | ND | ND | ND | ND | ND | 5.5 | 384 |
| 39 | 2 | 1.5 | ND | ND | ND | ND | ND | 7 | 906 |
| 40 | >200 | 76 | ND | ND | ND | ND | ND | 1 | 416 |
| 41 | 5.5 | 1 | ND | ND | ND | ND | ND | <1 | 405 |
| 42 | 57 | 12 | ND | ND | ND | ND | ND | 4 | 651 |
| 43 | 5 | 3 | ND | ND | ND | ND | ND | 1.5 | 180 |
| 44 | 2 | 1 | ND | ND | ND | ND | ND | 2.5 | 95 |
| 45 | 4.5 | 19 | ND | ND | ND | ND | ND | 2 | 490 |
| 46 | 15 | 5 | ND | ND | ND | ND | ND | 2.5 | 658 |
| 47 | 9.5 | 4 | ND | ND | ND | ND | ND | 2.5 | 345 |
| 48 | 14 | 6 | 488 | 140 | ND | ND | ND | 3 | 945 |
| 49 | 9.1 | 4 | ND | ND | ND | ND | ND | 1 | 4.3 |
| 50 | 23 | 17 | ND | ND | ND | ND | ND | 1 | ND |
| 51 | 91 | 42 | ND | ND | ND | ND | ND | 1 | ND |
| 52 | 39 | 13 | ND | ND | ND | ND | ND | 6.5 | ND |
| 53 | 2.5 | 4 | ND | ND | ND | ND | ND | 9.5 | 249 |
| 54 | 3 | 3.5 | ND | ND | ND | ND | ND | 7 | 248 |
| 55 | 6.5 | 16 | ND | ND | ND | ND | ND | 0.6 | 1687 |
| 56 | >200 | 200 | ND | ND | ND | ND | ND | >5000 | >30000 |
| 57 | 6.5 | 12 | ND | ND | ND | ND | ND | 1 | 249 |
| 58 | >200 | 200 | ND | ND | ND | ND | ND | 4520 | >30000 |
| 59 | 6 | 22 | ND | ND | ND | ND | ND | 1 | 1312 |
| 60 | 52 | 55 | ND | ND | ND | ND | ND | 18 | 34 |
| 61 | 98 | 54 | >5000 | 2779 | ND | ND | ND | ND | ND |
| 62 | 14 | 4 | ND | ND | ND | ND | ND | ND | ND |
| 63 | 11 | 4 | 655 | 190 | ND | ND | ND | ND | ND |
| 64 | >200 | 32 | ND | ND | ND | ND | ND | ND | ND |
| 65 | 17 | 15 | 791 | 169 | ND | ND | ND | ND | ND |
| 66 | 7 | 6 | 1719 | 426 | ND | 83 | ND | ND | ND |
| 67 | >140 | >200 | ND | ND | ND | ND | ND | ND | ND |
| 68 | >200 | >200 | ND | ND | ND | ND | ND | ND | ND |
| 69 | 12 | 7 | 683 | 252 | ND | ND | ND | ND | ND |
| 70 | 12 | 6 | 354 | 27 | ND | ND | ND | ND | ND |
| 71 | ND | 5 | ND | ND | ND | ND | ND | ND | ND |
| 72 | ND | 1 | ND | ND | ND | ND | ND | ND | ND |
| 73 | 15 | 28 | ND | ND | ND | ND | ND | ND | ND |
| 75 | 7 | 5.5 | ND | ND | ND | ND | ND | ND | ND |
| 76 | 9.5 | 6 | ND | ND | ND | ND | ND | ND | ND |
| 77 | 132 | 43 | ND | ND | ND | ND | ND | ND | ND |
| 78 | 33 | 15 | ND | ND | ND | ND | ND | ND | ND |
| 79 | >147 | 38 | ND | ND | ND | ND | ND | ND | ND |
| 80 | 128 | 22 | ND | ND | ND | ND | ND | ND | 295 |
| 81 | >200 | 137 | ND | ND | ND | ND | ND | ND | ND |
| 82 | >200 | 131 | ND | ND | ND | ND | ND | ND | ND |
| 83 | 84 | 24 | ND | ND | ND | ND | ND | ND | ND |
| 84 | >200 | 116 | ND | ND | ND | ND | ND | ND | ND |
| 85 | >200 | >190 | ND | ND | ND | ND | ND | ND | ND |
| 86 | 177 | 25 | ND | ND | ND | ND | ND | ND | 840 |
| 87 | 58 | 21 | >5000 | >3639 | ND | ND | ND | ND | 549 |
| 88 | 34 | 12 | >5000 | 1096 | ND | ND | ND | ND | 905 |
| 89 | 20 | 10 | >5000 | 874 | ND | ND | ND | ND | 751 |
| 90 | 132 | 115 | >5000 | 619 | ND | ND | ND | ND | 4939 |
| 91 | 84 | 28 | 913 | 79 | ND | ND | ND | ND | 705 |
| 92 | 15 | 6 | 118 | 16 | ND | ND | ND | ND | 725 |
| 93 | 2.6 | 4 | 53 | 2.5 | ND | ND | ND | ND | 12055 |
| 94 | >200 | >200 | >5000 | >5000 | ND | ND | ND | ND | >50000 |
| 95 | >200 | >184 | >5000 | 2042 | ND | ND | ND | ND | 4700 |
| 96 | >200 | 154 | ND | ND | ND | ND | ND | ND | >50000 |
| 97 | >200 | >200 | ND | ND | ND | ND | ND | ND | >50000 |
| 98 | >200 | >200 | ND | ND | ND | ND | ND | ND | >50000 |
| 101 | >200 | >200 | >5000 | 1743 | ND | ND | ND | 55 | 7774 |
| 104 | >180 | 56 | ND | 140 | ND | ND | ND | ND | 75 |
| 105 | 69 | 34 | >3400 | ND | ND | ND | ND | ND | 456 |
| 202 | 58 | 26 | ND | 154 | ND | ND | ND | ND | 39 |
| 303 | 3 | 3 | 97 | 4.5 | ND | ND | ND | ND | ND |
| 325 | 70 | 36 | ND | 382 | ND | ND | ND | ND | 22 |
| 326 | >200 | >200 | >5000 | >5000 | ND | ND | ND | ND | >50000 |
| 327 | 26 | 9 | >5000 | ND | ND | ND | ND | ND | 5686 |
| 341 | 3.5 | 8.5 | 197 | 7 | ND | ND | ND | 166 | >50000 |
| 342 | 10 | 22 | 439 | 41 | ND | ND | ND | <1 | 421 |
| 343 | >200 | >200 | >5000 | >3117 | ND | ND | ND | 6.5 | 770 |
| 344 | 77.5 | 64 | >4670 | 201 | ND | ND | ND | 3.5 | 1635 |
| 345 | 40 | 55 | 1108 | 30 | ND | ND | ND | 674 | >50000 |
| 346 | >200 | 57 | >5000 | 177 | ND | ND | ND | 18 | 10188 |
| 347 | >173 | >174 | 1533 | 668 | ND | ND | ND | 3 | ND |
| 348 | 62 | 55 | 1376 | 219 | ND | ND | ND | 31 | ND |
| 349 | 29 | 62 | 735 | 475 | ND | ND | ND | ND | ND |
| 350 | >200 | >200 | 2266 | 235 | ND | ND | ND | >5000 | ND |
| 351 | 48 | 15 | 117 | 18 | ND | ND | ND | 1155 | ND |
| 352 | 69 | 21 | 143 | 29 | ND | ND | ND | >5000 | ND |
| 353 | 15 | 13 | >2595 | 10 | ND | ND | ND | 69 | ND |
| 354 | 12 | 12 | 534 | 176 | ND | ND | ND | 3.5 | ND |
| 355 | 6.5 | 8 | 362 | 20 | ND | ND | ND | ND | ND |
| 356 | 2 | 4.5 | 55 | 5.5 | ND | ND | ND | 55 | 19860 |
| 357 | 2.7 | 5.8 | 230 | 11 | ND | ND | ND | 490 | >25345 |
| 358 | 4.4 | 6.6 | 1382 | 119 | ND | ND | ND | 1126 | >44035 |
| 359 | 1.4 | 12 | >5000 | 1875 | ND | ND | ND | >5000 | ND |
| 360 | 1.5 | 13 | 598 | <1 | ND | ND | ND | >5000 | ND |
| 361 | 1.3 | 7.1 | 157 | <1 | ND | ND | ND | 539 | ND |
| 362 | 6 | 15 | 1278 | 82 | ND | ND | ND | 90 | ND |
| 363 | 1.7 | 12 | 233 | 17 | ND | ND | ND | 138 | ND |
| 364 | >200 | >200 | >5000 | >5000 | ND | ND | ND | 64 | 825 |
| 365 | 167 | >200 | >5000 | 1245 | ND | ND | ND | 311 | 1711 |
| 366 | 22 | 4.5 | 2932 | 151 | ND | ND | ND | 925 | >48160 |
| 367 | 55 | 7.4 | 2918 | 308 | ND | ND | ND | 102 | 539 |
| 368 | 34 | 3.4 | 331 | 28 | ND | ND | ND | ND | 1979 |
| 369 | 7 | 5.5 | 87 | 7 | ND | ND | ND | ND | ND |
| 370 | 124 | 47 | 2734 | 242 | ND | ND | ND | ND | ND |
| 371 | 14 | 25 | >5000 | 3406 | ND | ND | ND | ND | ND |
| 372 | >200 | >200 | >4172 | 705 | ND | ND | ND | 1795 | ND |
| 373 | 4 | 4 | 912 | 42 | ND | ND | ND | 1438 | ND |
| 374 | 5 | 6 | 667 | 70 | ND | ND | ND | 838 | ND |
| 375 | 132 | 147 | 1309 | 303 | ND | ND | ND | 13 | ND |
| 376 | 159 | 58 | 1455 | 170 | ND | ND | ND | 402 | ND |
| 377 | 15 | 11 | 420 | 25 | ND | ND | ND | 27 | ND |
| 378 | 3 | 5 | 325 | 30 | ND | ND | ND | 402 | ND |
| 379 | 16 | 67 | >3619 | 434 | ND | ND | ND | ND | ND |
| 380 | 30 | 15 | 55 | 5.5 | ND | ND | ND | ND | ND |
| 381 | >200 | 120 | >3569 | 57 | ND | ND | ND | ND | ND |
| 382 | >200 | >200 | >4865 | 209 | ND | ND | ND | ND | ND |
| 383 | 11 | 6 | 233 | 8 | ND | ND | ND | ND | ND |
| 384 | 24 | 20 | 585 | 28 | ND | ND | ND | ND | ND |
| 385 | 49 | 65 | 2617 | 96 | ND | ND | ND | ND | ND |
| 386 | 5 | 4 | 118 | 9.5 | ND | ND | ND | ND | ND |
| 387 | 20 | 17 | 1097 | 88 | ND | ND | ND | ND | ND |
| 388 | 11 | 9 | ND | ND | ND | ND | ND | ND | ND |
| 389 | 7.5 | 11 | ND | ND | ND | ND | ND | ND | ND |
| 196 | 2.5 | 4.5 | 31 | 5 | ND | ND | ND | ND | ND |
| 401 | 4 | 4.5 | 88 | 19 | ND | ND | ND | ND | ND |
| 396 | 17 | 7 | 100 | 14 | ND | ND | ND | ND | ND |
| 395 | 13 | 11 | 66 | 18 | ND | ND | ND | ND | ND |

ND: Not Determined

### Example B2. Determination of Potency of Compounds in Cancer Cell Proliferation Assay as a Single Agent

The effects of test compounds were studied in three breast cancer cell lines of different subtype. The cancer cells (Table 3) were harvested during the logarithmic growth period and counted. Cell concentrations were adjusted to the appropriate number with respective medium and 90 μL cell suspensions were added to 96-well plates. After cells were seeded, the plates were shaken gently to distribute cells evenly and incubated at 37° C., 5% CO₂ on day 1.

TABLE 3
Cell Culture Conditions

| No. | Cell Line | Histopathology | Rb Status | Medium |
|---|---|---|---|---|
| 1 | MCF-7 | Breast adenocarcinoma | Positive | MEM + 10% FBS |
| 2 | DU4475 | Breast carcinoma | Negative | RPMI1640 + |
| | | | | 10% FBS |
| 3 | BT-549 | Breast ductal | Negative | RPMI1640 + |
| | | carcinoma | | 10% FBS |

Cells were treated with test compounds at 7 to 9 concentrations within a desired concentration range (e.g. 1.1 nM-10 μM) on day 2 by series diluting the test compound stock solution (10 mM in DMSO) with culture medium. Treatment duration was 24 Hr for BT-549 cells and 144 Hr (with a medium change at 72 Hr) for MCF-7 and DU4475 cells. Cell viability was assessed by Cell Titer-Glo® as recommended by Promega (Cat. No.: G7572), or by resazurin assay (Sigma Aldrich, Cat. No.: R7017) post treatment.

Cell viability data were plotted using GraphPad Prism (GraphPad Software, Inc., San Diego, Calif.). In addition, a nonlinear regression model with a sigmoidal dose response and variable slope within GraphPad Prism was used to calculate the IC₅₀ value of individual test compounds. IC₅₀ values are given in Table 4.

TABLE 4

| | MCF-7 | DU4475 | BT-549 |
|---|---|---|---|
| Compound | IC₅₀ | IC₅₀ | IC₅₀ |
| No. | (nM) | (nM) | (nM) |
| 1 | 8055 | ND | ND |
| 6 | 1155 | ND | ND |
| 10 | 90 | 1640 | ND |
| 12 | 2505 | ND | ND |
| 13 | 158 | ND | 1400 |
| 16 | 6025 | ND | ND |
| 17 | 3230 | ND | ND |
| 18 | 1420 | ND | ND |
| 19 | 2365 | ND | ND |
| 20 | 1050 | ND | ND |
| 21 | 238 | ND | ND |
| 22 | 1020 | ND | ND |
| 23 | 750 | 12425 | ND |
| 24 | >30000 | ND | ND |
| 25 | >30000 | ND | ND |
| 26 | 533 | ND | ND |
| 27 | 138 | ND | ND |
| 29 | 240 | 1285 | ND |
| 30 | 110 | ND | ND |
| 31 | 130 | 525 | 1110 |
| 32 | 2010 | ND | ND |
| 33 | 155 | ND | ND |
| 34 | 315 | ND | ND |
| 35 | 3878 | ND | ND |
| 36 | 315 | ND | ND |
| 37 | 353 | 2225 | ND |
| 38 | 452 | ND | ND |
| 39 | 2275 | ND | ND |
| 40 | 133 | 2795 | ND |
| 41 | 164 | ND | ND |
| 42 | 713 | ND | ND |
| 43 | 180 | ND | ND |
| 44 | 95 | ND | ND |
| 45 | 428 | ND | ND |
| 46 | 275 | ND | ND |
| 47 | 345 | ND | ND |
| 48 | 945 | ND | ND |
| 49 | 4435 | ND | ND |
| 50 | 395 | 2440 | ND |
| 51 | 570 | ND | ND |
| 52 | 6310 | ND | ND |
| 53 | 190 | 1280 | ND |
| 54 | 150 | 1095 | ND |
| 55 | 480 | 1260 | ND |
| 57 | 1480 | 5180 | ND |
| 59 | 540 | 1320 | ND |
| 60 | 6895 | 5085 | ND |
| 61 | 2730 | 4475 | ND |
| 62 | 594 | 1260 | ND |
| 63 | 266 | 1435 | ND |
| 64 | 3588 | 4745 | ND |
| 65 | 569 | 1990 | ND |
| 66 | 568 | 975 | ND |
| 67 | 1644 | 4450 | ND |
| 68 | >10000 | >10000 | ND |
| 69 | 1987 | 1685 | ND |
| 70 | 818 | 1180 | ND |
| 71 | 793 | 1650 | ND |
| 72 | 330 | 985 | ND |
| 73 | 1592 | >8430 | ND |
| 75 | 598 | 1050 | ND |
| 76 | 793 | 660 | ND |
| 77 | 452 | 4555 | ND |
| 78 | 751 | 1670 | ND |
| 79 | 1841 | 4055 | ND |
| 80 | 498 | 870 | ND |
| 81 | 641 | 585 | ND |
| 82 | 305 | 250 | ND |
| 83 | 1064 | 820 | ND |
| 84 | 2002 | 2515 | ND |
| 85 | 723 | 725 | ND |
| 86 | 532 | 1395 | ND |
| 87 | ND | 6705 | ND |
| 88 | ND | 1565 | ND |
| 89 | ND | 5205 | ND |
| 90 | ND | 4895 | ND |
| 91 | ND | 510 | ND |
| 92 | ND | 420 | ND |
| 93 | 2005 | 793 | ND |
| 94 | 1945 | 485 | ND |
| 95 | 3575 | 1505 | ND |
| 96 | 2435 | 1695 | ND |
| 97 | 1610 | 1270 | ND |
| 98 | 1725 | 1430 | ND |
| 101 | >30000 | >30000 | ND |
| 104 | 1035 | 2520 | ND |
| 105 | 675 | 3820 | ND |
| 202 | 830 | 3760 | ND |
| 303 | 660 | 710 | ND |
| 325 | 445 | 2415 | ND |
| 326 | 3520 | 4240 | ND |
| 327 | 650 | 1705 | ND |
| 341 | 835 | 940 | ND |
| 342 | 415 | 485 | ND |
| 343 | 1280 | 5715 | ND |
| 344 | 435 | 960 | ND |
| 345 | 3145 | 3690 | ND |
| 346 | >30000 | 10090 | ND |
| 347 | 405 | 1240 | ND |
| 348 | 725 | 6840 | ND |
| 349 | 770 | 5660 | ND |
| 350 | >10000 | >30000 | ND |
| 351 | 205 | 1405 | ND |
| 352 | 1415 | 1130 | ND |
| 353 | 515 | 900 | ND |
| 354 | 120 | 320 | ND |
| 355 | 495 | 1515 | ND |
| 356 | 175 | 1145 | ND |
| 357 | 190 | 1305 | ND |
| 358 | 2800 | 12810 | ND |
| 359 | 330 | 3245 | ND |
| 360 | 255 | 1415 | ND |
| 361 | 310 | 1040 | ND |
| 362 | 765 | 2675 | ND |
| 363 | 265 | 1660 | ND |
| 364 | 3330 | 5665 | ND |
| 365 | 1255 | 5630 | ND |
| 366 | 320 | 2410 | ND |
| 367 | 640 | 5160 | ND |
| 368 | 405 | 1390 | ND |
| 369 | 107 | 670 | ND |
| 370 | 445 | 1225 | ND |
| 371 | 515 | 2605 | ND |
| 372 | ND | >10000 | ND |
| 373 | ND | 200 | ND |
| 374 | ND | 690 | ND |
| 375 | ND | 615 | ND |
| 376 | ND | 2445 | ND |
| 377 | ND | 365 | ND |
| 378 | ND | 670 | ND |
| 379 | 1219 | 4725 | ND |
| 380 | 1221 | 715 | ND |
| 381 | 743 | >10000 | ND |
| 382 | 1843 | >10000 | ND |
| 383 | 145 | 2445 | ND |
| 384 | 1605 | 5685 | ND |
| 385 | 2820 | >10000 | ND |
| 386 | 270 | 870 | ND |
| 387 | 580 | 7060 | ND |
| 388 | ND | >5260 | ND |
| 389 | ND | 4220 | ND |

ND: Not Determined

The effects of test compounds in a palbociclib-resistant cell line and a parental, non-resistant cell line were compared. The palbociclib-resistant cell line (“MCF-7-PR”) was derived from the parental, non-resistant cell line (MCF-7 breast adenocarcinoma cells) by culture of cells over a period of three months in increasing concentrations of palbociclib, starting from about 350 nM and ending at about 850 nM, the final concentration at which they were then maintained in culture. The MCF-7-PR cells were checked using a cell viability assay to confirm that they had at least 5-fold resistance to palbociclib compared to parental MCF-7 cells, as measured by an increase in the cell viability IC₅₀ values. Assessment of cell viability following treatment with palbociclib or test compounds was performed according to the method described above for MCF-7 cells. Results are shown in Table 5.

TABLE 5

| Compound | MCF-7 | MCF-7-PR |
|---|---|---|
| No. | IC₅₀ (nM) | IC₅₀ (nM) |
| 13 | 119 | 481 |
| 30 | 115 | 85 |
| 31 | 157 | 248 |
| 33 | 205 | 65 |
| 50 | 664 | 1040 |
| 93 | 558 | 911 |
| 341 | 487 | 690 |
| 342 | 192 | 361 |
| 345 | 3968 | 6230 |
| 353 | 347 | 769 |

The effects of test compounds are studied in additional cell lines of various histotypes, such as A549 lung adenocarcinoma, HCT-116 colorectal carcinoma, ZR-75-30 breast ductal carcinoma and Hs-578T breast epithelia carcinoma cells. The cancer cells are harvested during the logarithmic growth period and counted. Cell concentrations are adjusted to the appropriate number with suitable medium, and 90 μL cell suspensions are added to 96-well plates. After cells are seeded, the plates are shaken gently to distribute cells evenly and incubated at 37° C., 5% CO₂ on day 1. Cells are treated with test compounds at typically 7-9 concentrations within a desired concentration range (e.g. 1.5 nM-10 μM) on day 2 by series diluting the test compound stock solution (10 mM in DMSO) with culture medium. Cell viability is assessed by Cell Titer-Glo® as recommended by Promega (Cat. No.: G7572, Promega) typically 48-144H post-treatment, with a medium change as necessary. Cell viability data are plotted using GraphPad Prism (GraphPad Software, Inc., San Diego, Calif.). In addition, a nonlinear regression model with a sigmoidal dose response and variable slope within GraphPad Prism is used to calculate the IC₅₀ value of individual test compounds.

Additional test compounds are studied in the same and/or other cancer cell lines using similar proliferation methods with possible variations in cell seeding densities and/or incubation durations. The cell cycle phase distribution post treatment of test compounds is studied using flow cytometer using DAPI staining. Cellular senescence is evaluated after continuously treating cells for a long time (e.g., 14 days) followed by staining cells lines for Senescence associated-β-galactosidase (SAβGAL).

### Example B3. Determination of pRb Levels

Hypo-phosphorylation of the retinoblastoma protein (Rb) by cyclin D:Cdk4/6 complexes results in active Rb, which is a clinically relevant biomarker associated with CDK4 or CDK6 inhibition. As a confirmatory measure of functional activity of CDK4/6, the Ser780 phosphorylation state of Rb was assessed. MCF-7 cells were plated at 2.5×10⁵ to 3.0×10⁶ cells/well in 6-well cell-culture plates and incubated at 37° C. for 24H in MEM medium supplemented with 10% FBS. Cells were treated for 24H with a medium containing test compound at various concentrations (e.g., 0.01, 0.1, 1 M) or with DMSO (≤1%) in duplicate. After incubation period, the media was removed, and cells were rinsed once with ice-cold PBS and lysed with 0.2 mL of Cell Lysis Buffer containing 1 mM PMSF and Protease Inhibitor. Protein concentration was estimated following Bradford method. The lysis and the Rb measurements were performed following the manufacturer's ELISA kit protocols and buffers (Cell Signaling Technology, Cat. No.: 13016C). Rb inhibition of test compounds was calculated as percentage of vehicle control. In some cases, a nonlinear regression model with a sigmoidal dose response and variable slope within GraphPad Prism was used to calculate the IC₅₀ value of Rb inhibition for individual test compounds. Values are given in Table 6.

TABLE 6

| | | | | Inhibition of Rb |
|---|---|---|---|---|
| Compound | Inhibition of Rb phosphorylation (%) | | | phosphorylation |
| No. | 1 μM | 0.1 μM | 0.01 μM | IC₅₀ (nM) |
| 13 | 93 | 58 | 35 | ND |
| 29 | 44 | 27 | 12 | ND |
| 31 | 95 | 27 | 1 | ND |
| 39 | 52 | 40 | −25 | ND |
| 50 | ND | ND | ND | 505 |
| 93 | ND | ND | ND | <123 |

ND: Not Determined

The effects of selected test compounds in additional cancer cell lines on clinically relevant biomarkers associated with CDK4 or CDK6 inhibition (e.g., Rb and thymidine kinase (TK)) is assessed using ELISA or Western Blotting methods with selective antibodies.

### Example B4. Determination of Potency and Combination Effects of Compounds in Cancer Cell Proliferation Assays Using Combination Therapy

Effects of test compounds on cell proliferation is studied in additional cancer cell lines, such as estrogen receptor over-expressing cancer cells, in combination with another anti-cancer therapy (e.g., an aromatase inhibitor and/or a selective estrogen receptor degrader for breast cancer) using CTG, resazurin and/or Brdu assays. Cells seeded in a 96-well plate are treated with single agents to obtain a dose response curve for each agent. Cells are also treated with combinations of the drugs, based on a matrix generated by combining the two drugs at all different combinations of the doses used in the dose response curves. In place of a combination matrix method, a fixed drug ratio dilution method in which drugs are combined in a fixed ratio of 5 or more dilutions may also be used. The combined treatment effect, such as additive, synergistic, or antagonistic, is determined using the median-effect principle (Chou TC. Cancer Res 2010; 70:440-6.), with the combination index (CI) value indicating an additive effect (CI=1), synergism (CI<1), or antagonism (CI>1) in drug combinations.

### Example B5. In Vivo Pharmacology Studies in Xenograft or Syngeneic Models

The anti-tumor activity of test compounds is studied against various human tumor xenograft or syngeneic models in mice for example, in breast cancer tumor models. For breast cancer tumor models, effects of test compounds on Rb-Positive or Rb-Negative tumors as a single agent or in combination with another anti-cancer therapy is determined by evaluating the difference of tumor volume between treatment group against the vehicle control group. The phosphorylation status of serine-780 on Rb is evaluated in tumor tissue and compared with antitumor response in Rb-Positive xenograft model(s). Additional pharmacodynamic end points (e.g., FoxM1, E2F1, c-Myc, and cyclin D1) are studied in tumor tissues collected at various time points post treatment. Induction of senescence is evaluated in tumor samples from various treatment groups by measuring SAβGAL.

### Example B6. In Vivo Pharmacology Study in MC-38 Mouse Model

The therapeutic efficacy of test compound in the treatment of the MC-38 murine colorectal cancer model is evaluated in combination with an anti mPD-1 antibody. Cultured MC-38 cells are harvested and re-suspended in base medium at a density of 1×10⁷ cells/mL with viability greater than 90%. Female C57BL/6 mice are inoculated subcutaneously at the right flank with 1×10⁶ cells in 0.1 mL base medium for tumor development. The mice are stratified into treatment groups and the treatments are started after tumor inoculation when the tumor size reaches, for example, 45-72 mm³ (average tumor size 56 mm³). Tumors are measured using a caliper and tumor volumes calculated using the formula: Tumor volume=(a×b²/2) where ‘b’ is the smallest diameter and ‘a’ is the largest diameter. The treatment groups are, for example: vehicle control, test compound alone, anti mPD-1 alone, and test compound+anti mPD-1 at 10 mice per group. The exact treatment groups, drug dose, and dosing schedule are determined specifically for each study according to standard practice. Tumor growth is monitored, and volume recorded at regular intervals. When the individual tumor of each mouse reaches an approximate end-point (for example, tumor volume>2,000 mm³), the mouse is sacrificed. The tumor growth inhibition (TGI) is calculated by comparing the control group's tumor measurements with the other study groups once the predetermined endpoint is reached in the control group.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A compound of Formula (K): 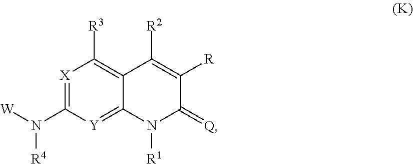
or a salt thereof, wherein:
X is CR^{a} or N, wherein R^{a} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²;
Y is CR^{b} or N, wherein R^{b} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²,
provided that at least one of X and Y is N;
Q is O or S;
W is 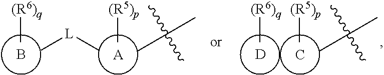
wherein:
A is C₃-C₆ cycloalkyl, 4- to 7-membered heterocyclyl, 5- to 7-membered heteroaryl, or phenyl, each of which is independently optionally substituted by R⁵,
L is a bond, —CR¹¹R¹²—, —O—, —S—, —S(O)₂—, —C(O)—, —NR¹⁰—, —S(O)₂NR¹⁰—, or —NR¹⁰S(O)₂—,
B is hydrogen, C₃-C₆ cycloalkyl, 3- to 10-membered heterocyclyl, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶,
C is C₃-C₆ cycloalkyl, 5- to 7-membered heterocyclyl, 5- to 7-membered heteroaryl, or phenyl, each of which is independently optionally substituted by R⁵, wherein C is fused to D, and
D is C₃-C₆ cycloalkyl, 3- to 7-membered heterocyclyl, 5- to 7-membered heteroaryl, or phenyl, each of which is independently optionally substituted by R⁶;
R is —CN, C₁-C₆ haloalkyl, or C₃-C₆ cycloalkyl;
R¹ is C₁-C₆ alkyl, C₃-C₁₂ cycloalkyl, 3- to 12-membered heterocyclyl, 5- to 10-membered heteroaryl, C₆-C₁₄ aryl, —(C₁-C₃ alkylene)(C₃-C₆ cycloalkyl), —(C₁-C₃ alkylene)(3- to 12-membered heterocyclyl), —C(O)R¹⁰, —(C₁-C₃ alkylene)(5- to 10-membered heteroaryl), or —(C₁-C₃ alkylene)(C₆-C₁₄ aryl), each of which is independently optionally substituted by halogen, oxo, —OR¹³, —NR¹³R¹⁴, —C(O)R¹³, —CN, C₃-C₈ cycloalkyl, or C₁-C₆ alkyl optionally substituted by oxo, —OH or halogen;
R² and R³ are each independently hydrogen, C₁-C₆ alkyl, C₃-C₆ cycloalkyl, C₁-C₆ haloalkyl, C₁-C₆ alkoxy, C₁-C₆ haloalkoxy, halogen, —CN, —C(O)R¹⁰, or —C(O)NR¹¹R¹²;
R⁴ is hydrogen or C₁-C₆ alkyl;
each R⁵ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen;
each R⁶ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen,
or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each optionally substituted by C₁-C₆ alkyl;
R¹⁰ is independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR¹⁵, —NR¹⁵R¹⁶, or C₁-C₆ alkyl optionally substituted by halogen, —OH or oxo;
R¹¹ and R¹² are each independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR, —NR¹⁵R¹⁶ or C₁-C₆ alkyl optionally substituted by halogen, —OH or oxo,
or R¹¹ and R¹² are taken together with the atom to which they attached to form a 3- to 6-membered heterocyclyl optionally substituted by halogen, oxo, or C₁-C₆ alkyl optionally substituted by halogen;
R¹³ and R¹⁴ are each independently hydrogen, —OH, C₁-C₆ alkoxy, or C₁-C₆ alkyl, wherein the C₁-C₆ alkyl of R¹³ and R¹⁴ are optionally substituted by halogen, —OR, —NR¹⁵R¹⁶, or oxo,
or R¹³ and R¹⁴ are taken together with the atom to which they attached to form a 3- to 6-membered heterocyclyl optionally substituted by halogen, oxo or C₁-C₆ alkyl optionally substituted by halogen or oxo;
R¹⁵ and R¹⁶ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo,
or R¹⁵ and R¹⁶ are taken together with the atom to which they attached to form a 3- to 6-membered heterocyclyl optionally substituted by halogen, oxo or C₁-C₆ alkyl optionally substituted by oxo or halogen;
p and q are each independently 0, 1, 2, 3 or 4.

2. The compound of claim 1, wherein the compound is a compound of Formula (J): 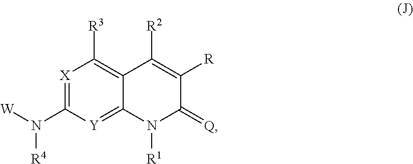
or a salt thereof, wherein:
X is CR^{a} or N, wherein R^{a} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²;
Y is CR^{b} or N, wherein R^{b} is hydrogen, C₁-C₆ alkyl, C₃-C₆ cycloalkyl, C₁-C₆ haloalkyl, C₁-C₆ alkoxy, C₁-C₆ haloalkoxy, halogen, —NR¹¹R¹², —CN, —C(O)R¹⁰, or —C(O)NR¹¹R¹²,
provided that at least one of X and Y is N;
Q is O or S;
W is 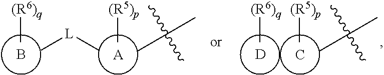
wherein:
A is C₃-C₆ cycloalkyl, 4- to 7-membered heterocyclyl, 5- to 7-membered heteroaryl, or phenyl, each of which is independently optionally substituted by R⁵,
L is a bond, —CR¹¹R¹²—, —O—, —S—, —S(O)₂—, —C(O)—, —NR¹⁰—, —S(O)₂NR¹⁰—, or —NR¹⁰S(O)₂—,
B is hydrogen, C₃-C₆ cycloalkyl, 3- to 10-membered heterocyclyl, 5- to 7-membered heteroaryl, or phenyl, wherein the C₃-C₆ cycloalkyl, 3- to 10-membered heterocyclyl, 5- to 7-membered heteroaryl, and phenyl of B are optionally substituted by R⁶,
C is C₃-C₆ cycloalkyl, 5- to 7-membered heterocyclyl, 5- to 7-membered heteroaryl, or phenyl, each of which is independently optionally substituted by R⁵, wherein C is fused to D, and
D is C₃-C₆ cycloalkyl, 3- to 7-membered heterocyclyl, 5- to 7-membered heteroaryl, or phenyl, each of which is independently optionally substituted by R⁶;
R is —CN or C₁-C₆ haloalkyl;
R¹ is C₁-C₆ alkyl, C₃-C₆ cycloalkyl, 3- to 12-membered heterocyclyl, 5- to 10-membered heteroaryl, C₆-C₁₄ aryl, —(C₁-C₃ alkylene)(C₃-C₆ cycloalkyl), —(C₁-C₃ alkylene)(3- to 12-membered heterocyclyl), —C(O)R¹⁰, —(C₁-C₃ alkylene)(5- to 10-membered heteroaryl), or —(C₁-C₃ alkylene)(C₆-C₁₄ aryl), each of which is independently optionally substituted by halogen, oxo, —OR¹³, —NR¹³R¹⁴, —C(O)R¹³, —CN, C₃-C₈ cycloalkyl, or C₁-C₆ alkyl optionally substituted by oxo, —OH or halogen;
R² and R³ are each independently hydrogen, C₁-C₆ alkyl, C₃-C₆ cycloalkyl, C₁-C₆ haloalkyl, C₁-C₆ alkoxy, C₁-C₆ haloalkoxy, halogen, —CN, —C(O)R¹⁰, or —C(O)NR¹¹R¹²;
R⁴ is hydrogen or C₁-C₆ alkyl;
each R⁵ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen;
each R⁶ is independently C₁-C₆ alkyl, C₂-C₆ alkenyl, C₂-C₆ alkynyl, C₁-C₆ haloalkyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, 3- to 12-membered heterocyclyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen,
or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each optionally substituted by C₁-C₆ alkyl;
R¹⁰ is independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR¹⁵, —NR¹⁵R¹⁶, or C₁-C₆ alkyl optionally substituted by halogen, —OH or oxo;
R¹¹ and R¹² are each independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, or 3- to 6-membered heterocyclyl, each of which is independently optionally substituted by halogen, oxo, —CN, —OR, —NR¹⁵R¹⁶ or C₁-C₆ alkyl optionally substituted by halogen, —OH or oxo,
or R¹¹ and R¹² are taken together with the atom to which they attached to form a 3- to 6-membered heterocyclyl optionally substituted by halogen, oxo, or C₁-C₆ alkyl optionally substituted by halogen;
R¹³ and R¹⁴ are each independently hydrogen, —OH, C₁-C₆ alkoxy, or C₁-C₆ alkyl, wherein the C₁-C₆ alkyl of R¹³ and R¹⁴ are optionally substituted by halogen, —OR, —NR¹⁵R¹⁶, or oxo,
or R¹³ and R¹⁴ are taken together with the atom to which they attached to form a 3- to 6-membered heterocyclyl optionally substituted by halogen, oxo or C₁-C₆ alkyl optionally substituted by halogen or oxo;
R¹⁵ and R¹⁶ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo,
or R¹⁵ and R¹⁶ are taken together with the atom to which they attached to form a 3- to 6-membered heterocyclyl optionally substituted by halogen, oxo or C₁-C₆ alkyl optionally substituted by oxo or halogen;
p and q are each independently 0, 1, 2, 3 or 4.

3. The compound of claim 1, or a salt thereof, wherein X is CR^{a}.

4. The compound of claim 1, or a salt thereof, wherein X is N.

5. The compound of claim 1, or a salt thereof, wherein Y is CR^{b}.

6. The compound of claim 1, or a salt thereof, wherein Y is N.

7. The compound of claim 1, or a salt thereof, wherein Q is O.

8. The compound of claim 1, or a salt thereof, wherein R is —CN.

9. The compound of claim 1, or a salt thereof, wherein the compound is of Formula (I),

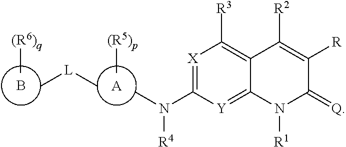

10. The compound of claim 1, or a salt thereof, wherein the compound is of Formula (II),

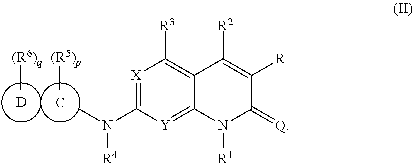

11. The compound of claim 1, or a salt thereof, wherein R¹ is C₁-C₆ alkyl, C₃-C₆ cycloalkyl, 3- to 12-membered heterocyclyl, —(C₁-C₃ alkylene)(C₆-C₁₄ aryl), C₆-C₁₄ aryl, or —(C₁-C₃ alkylene)(C₃-C₆ cycloalkyl), each of which is independently optionally substituted by halogen, —OR¹³, or C₁-C₆ alkyl optionally substituted by oxo, —OH, or halogen.

12. The compound of claim 1, or a salt thereof, wherein R² is hydrogen.

13. The compound of claim 1, or a salt thereof, wherein R³ is hydrogen.

14. The compound of claim 1, or a salt thereof, wherein R⁴ is hydrogen,

15. The compound of claim 1, or a salt thereof, wherein the compound is of any one of Fomulae (I-B1) to (I-B22),

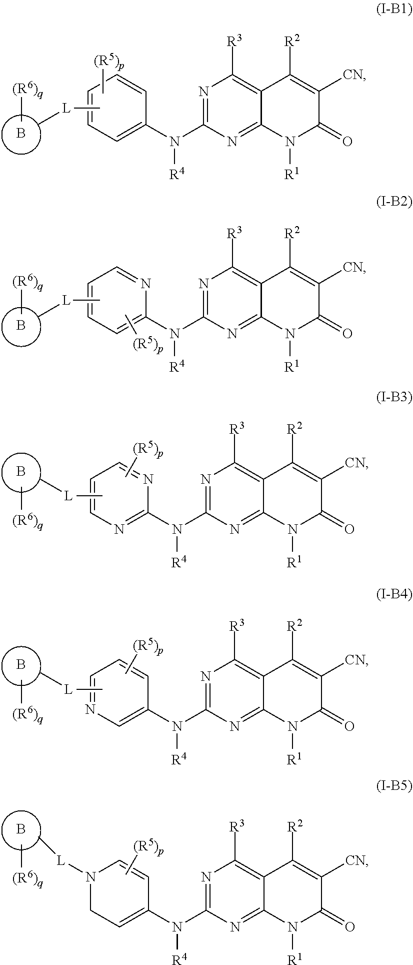

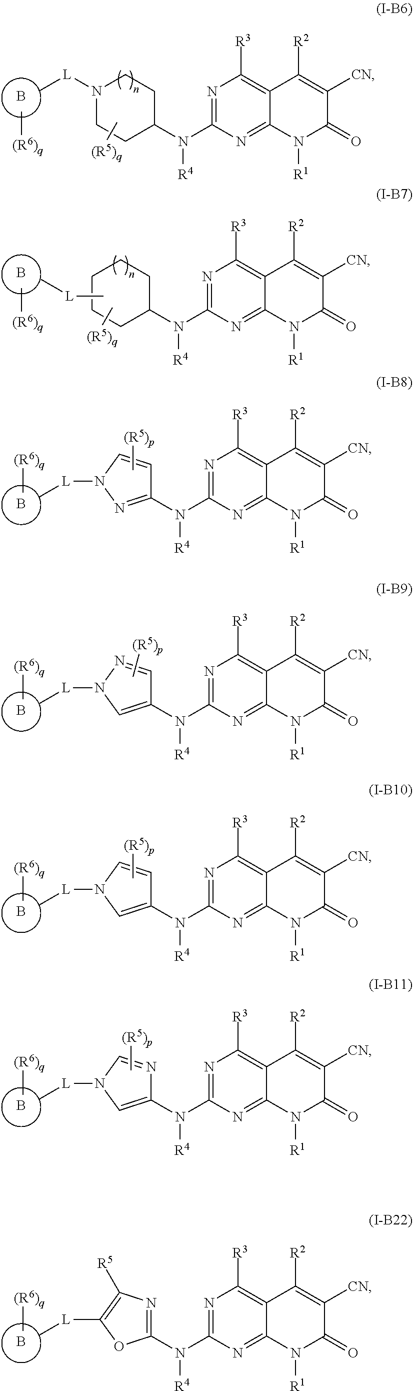

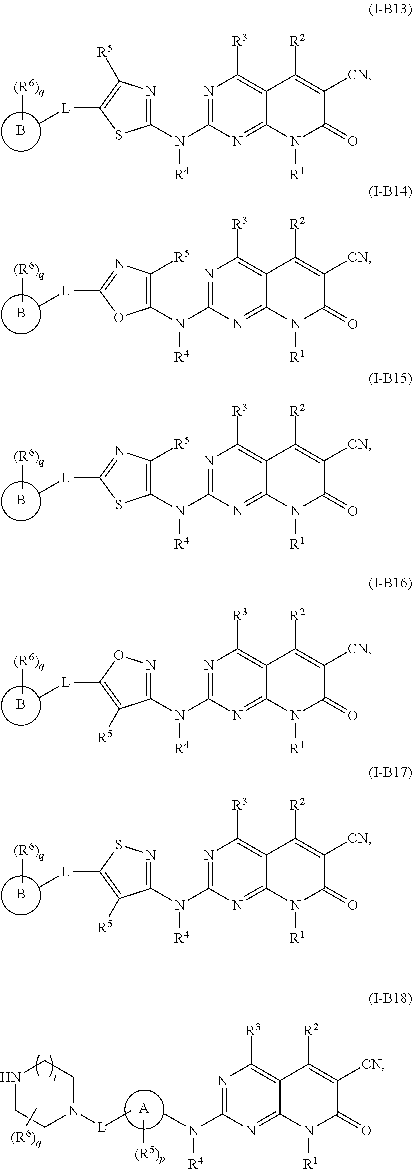

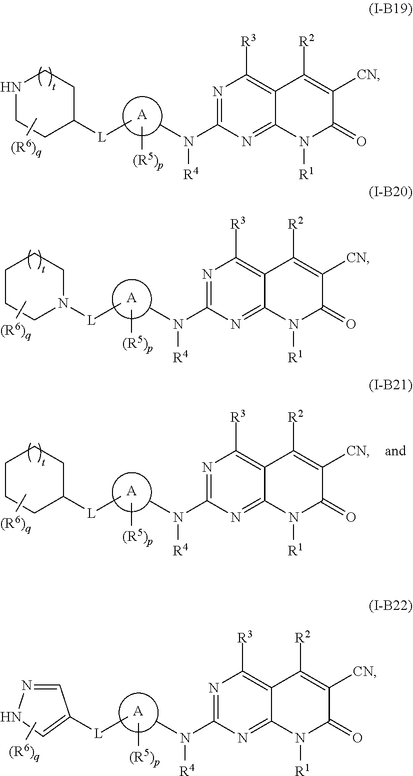

16. The compound of claim 1, or a salt thereof, wherein L is a bond.

17. The compound of claim 1, or a salt thereof, wherein the compound is of any one of Formulae (I-C1) to (I-C23): 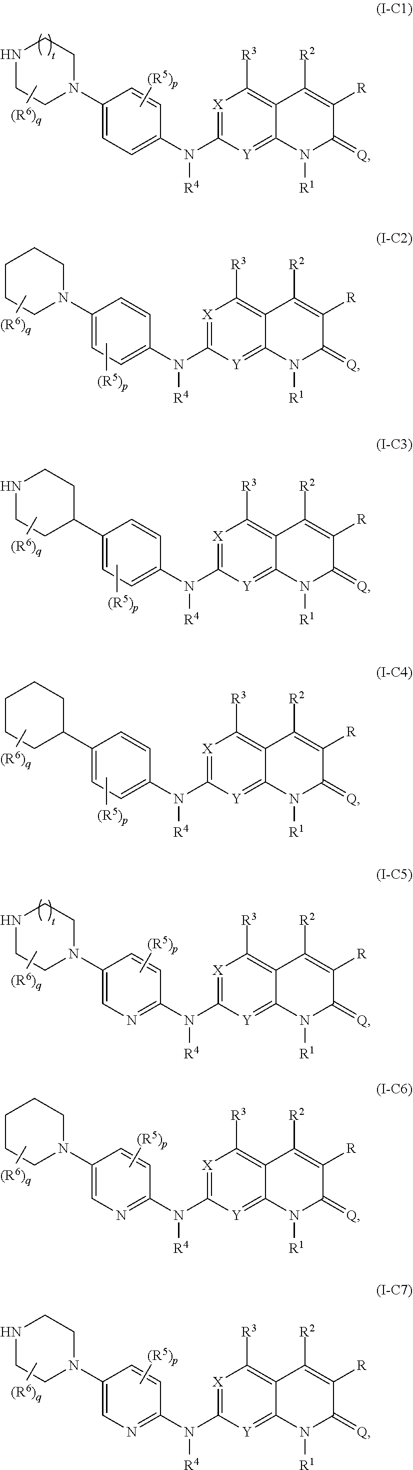 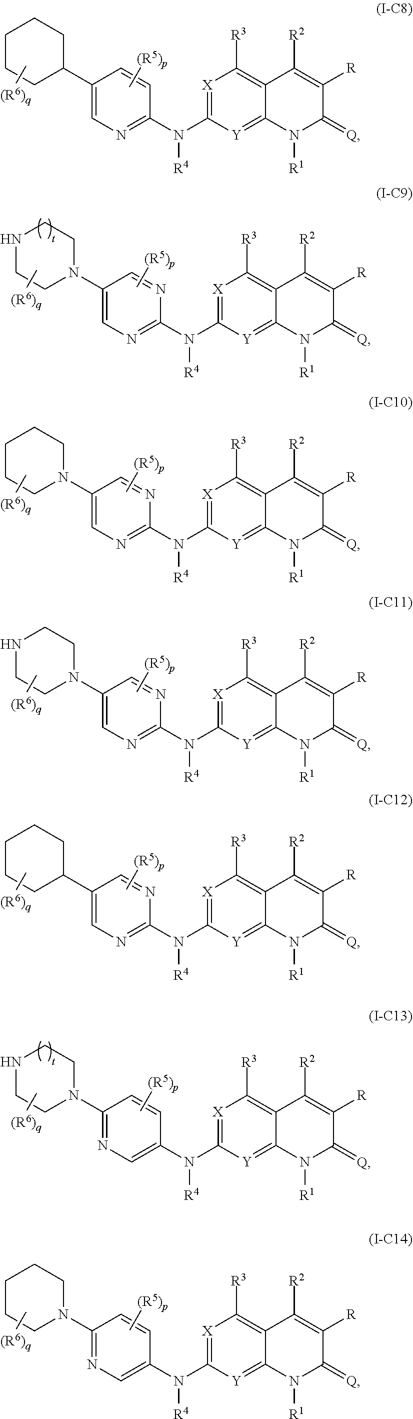 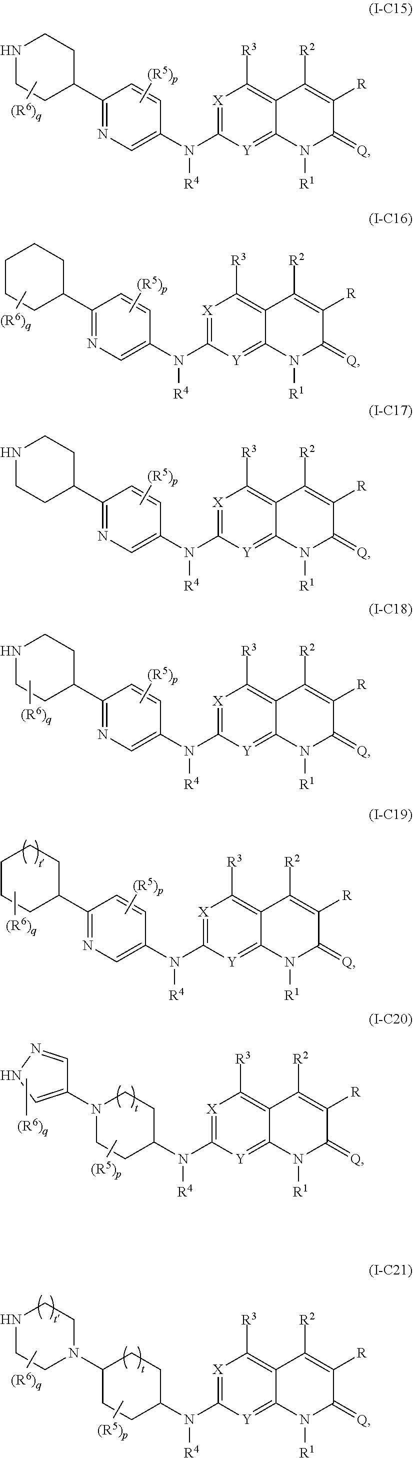 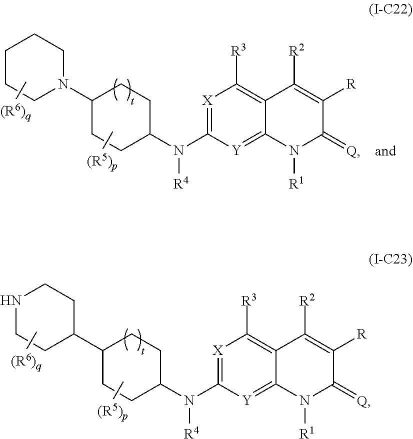
wherein t and t′ are each independently 0, 1, 2, or 3.

18. The compound of claim 1, or a salt thereof, wherein C, D, R⁵, and R⁶ together form a moiety selected from the group consisting of:

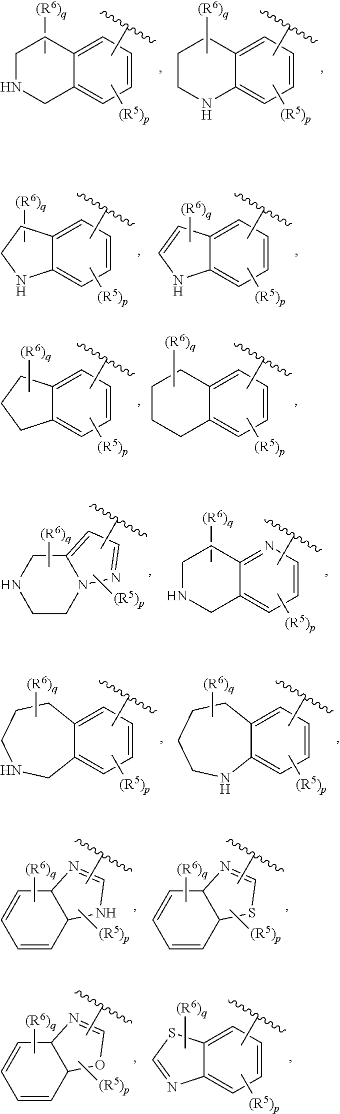

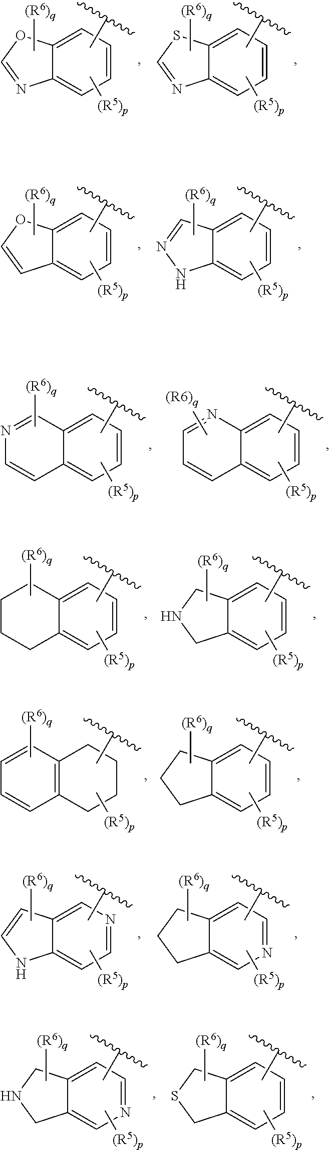

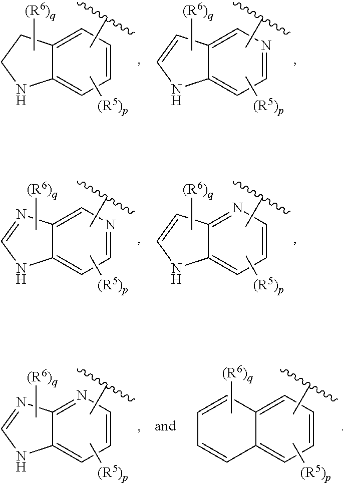

19. The compound of claim 1, or a salt thereof, wherein each R⁵ is independently —S(O)₂R¹⁰, —S(O)₂NR¹¹R¹², —C(O)NR¹¹R¹², —(C₁-C₃ alkylene)OR¹⁰, 3- to 12-membered heterocyclyl, —(C₁-C₃ alkylene)NR¹¹R¹², halogen, C₁-C₆ alkyl, —OR¹⁰, or oxo, each of which is independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen.

20. The compound of claim 1, or a salt thereof, wherein each R⁶ is independently C₁-C₆ alkyl, oxo, —OR¹⁰, —(C₁-C₃ alkylene)NR¹¹R¹², C₃-C₆ cycloalkyl, 3- to 12-membered heterocyclyl, —S(O)₂NR¹¹R¹², —NR¹¹R¹², —C(O)R¹⁰, —(C₁-C₃ alkylene)C(O)NR¹¹R¹², —S(O)₂R¹⁰, —(C₁-C₃ alkylene)(C₃-C₆ cycloalkyl), —(C₁-C₃ alkylene)(3- to 12-membered heterocyclyl), or —(C₁-C₃ alkylene)OR¹⁰, each of which is independently optionally substituted by halogen, oxo, —OR¹, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, —(C₁-C₃ alkylene)S(O)₂R¹³, C₃-C₈ cycloalkyl, or C₁-C₆ alkyl optionally substituted by oxo, —OH or halogen;
or any two R⁶ groups are taken together with the atom or atoms to which they are attached to form a C₃-C₆ cycloalkyl or 3- to 12-membered heterocyclyl, wherein the C₃-C₆ cycloalkyl or 3- to 12-membered heterocyclyl are each independently optionally substituted by C₁-C₆ alkyl.

21. A compound selected from the group of the compounds in Table 1, or a salt thereof.

22. A pharmaceutical composition comprising the compound of claim 1, or a pharmaceutically acceptable salt thereof, and a pharmaceutically acceptable carrier.

23. A method of treating a cancer in an individual in need thereof comprising administering to the individual a therapeutically effective amount of a compound of claim 1, or a pharmaceutically acceptable salt thereof.

24. The method of claim 23, where the cancer is a breast cancer, brain cancer, colorectal cancer, lung cancer, gastric cancer, liver cancer, leukemia, lymphoma, mantle cell lymphoma, melanoma, ovarian cancer, pancreatic cancer, prostate cancer, adult hematopoietic or solid tumor, or pediatric tumor.

25. The method of claim 23, further comprising administering a radiation therapy to the individual.

26. The method of claim 23, further comprising administering to the individual a therapeutically effective amount of a second therapeutic agent.

27. The method of claim 26, wherein the second therapeutic agent is a cancer immunotherapy agent, an endocrine therapy agent, or a chemotherapeutic agent.

28. The method of claim 26, wherein the second therapeutic agent is a cancer immunotherapy.

29. The method of claim 26, wherein the second therapeutic agent is an anti-PD-1 antibody.

30. The method of claim 27, wherein the endocrine therapy agent is an antiestrogen therapy, a selective estrogen receptor degrader (SERD), or an aromatase inhibitor.

31. The method of claim 27, wherein the chemotherapeutic agent is a DNA alkylating agent, a platinum-based chemotherapeutic agent, a taxane, a BTK inhibitor, a PI3K inhibitor, another kinase inhibitor, or a DNA damage repair (DDR) pathway inhibitor.

32. The method of claim 23, wherein the cancer comprises a mutated or overexpressed CDK gene.

33. The method of claim 23, comprising selecting the individual for treatment based on (i) the presence of one or more mutations or amplifications of the CDK4 or CDK6 or other CDK gene in the cancer, (ii) overexpression of CDK4 or CDK6 or other CDK protein in the cancer, (iii) amplification or overexpression of the genes encoding cyclins, (iv) loss of endogenous INK4 inhibitors by gene deletion, mutation, or promoter hypermethylation, (v) other genetic events leading to overactivity of CDK4 or CDK6 or other CDK, or (vi) phosphorylation of retinoblastoma (Rb) protein in the cancer.

34. A method of arresting the G₁-S checkpoint in a cell, comprising administering a compound of claim 1, or a salt thereof, to the cell.

35. A method of inducing senescence in a cell, comprising administering a compound of claim 1, or a salt thereof, to the cell.

36. A method of inducing apoptosis in a cell, comprising administering a compound of claim 1, or a salt thereof, to the cell.

37. A method of inhibiting CDK4 or CDK6 in a cell, comprising administering a compound of claim 1, or a salt thereof, to the cell.

38. A method of inhibiting CDK4 or CDK6, comprising contacting CDK4 or CDK6 with a compound of claim 1, or a salt thereof.

39. The method of claim 38, wherein the inhibitor binds to CDK4 or CDK6 with an IC₅₀ of less than 1 μM according to a kinase assay.

40. Use of a compound of claim 1, or a salt thereof, in the manufacture of a medicament for treatment of cancer.

41. A kit comprising a compound of claim 1, or a salt thereof.
